(19) United States
(12) Patent Application Publication
(10) Pub. No.: US 2014/0128385 A1
(43) Pub. Date: May 8, 2014

(54) BACE-2 INHIBITORS FOR THE TREATMENT OF METABOLIC DISORDERS

(71) Applicant: NOVARTIS AG, Basel (CH)

(72) Inventor: Heinrich Ruegger, Basel (CH)

(21) Appl. No.: 13/979,452

(22) PCT Filed: Jan. 13, 2012
(86) PCT No.: PCT/EP2012/050496

§ 371 Date: Jan. 9, 2014

Related U.S. Application Data
(60) Provisional application No. 61/432,510, filed on Jan. 13, 2011.

(51) Int. Cl. C07D 417/12; C07D 413/14; C07D 471/04; A61K 45/06; A61K 31/5377; C07D 413/12

(52) U.S. Cl. CPC C07D 417/12; A61K 31/5377; C07D 413/12; C07D 471/04; A61K 45/06; C07D 413/14
USPC 514/233.2; 514/231.5; 544/152; 514/235.5; 544/131; 514/236.8; 544/137; 514/235.8; 544/120; 544/122; 544/127; 544/133; 514/236.5; 544/114

(57) ABSTRACT

The present invention relates to the use of BACE-2 inhibitors and pharmaceutical compositions comprising BACE-2 inhibitors for treating metabolic disorders related to decreased β cell mass and/or function.

### BACKGROUND

In 2007, the American Diabetes Association (ADA) commissioned study reported on the economic cost of diabetes. The number of diagnosed diabetics was estimated to be 17.1 million with an economic cost of $174 billion. This number continues to steadily rise, driven by increases in sedentary lifestyle, obesity, aging of the population and is projected to increase 165% by 2050 in the U.S. The Centers for Disease Control and Prevention (CDC) recently referred to diabetes as “the epidemic of our time”. Many people with Type 2 Diabetes (T2D) will develop more than one health complications associated with the disease including macrovascular and microvascular complications.

Existing anti-diabetic medications fail to provide lasting metabolic control. Moreover, they do not address the underlying processes leading to the development of the disease, notably the decline in beta cell mass and function, the causal step in the development and progression of disease. As a result, therapeutic intervention with current anti-diabetic therapies including Metformin, sulphonylureas and even insulin cannot prevent progressive hyperglycemia and highlight the importance of identifying new medicines with disease modifying potential.

Beta-site amyloid precursor protein cleaving enzyme 2 (BACE-2) is a transmembrane aspartic protease that is highly expressed in pancreatic β cells and other peripheral tissues (Brian D. Bennett, Safura Babu-Khan, Richard Loeloff, Jean-Claude Louis, Eileen Curran; Martin Citron, and Robert Vassar (2000) JJ. Biol. Chem. 275(27) 20647-20651). BACE-2 is closely related to BACE or β secretase. However, despite structural and sequence similarities the substrate specificity of BACE and BACE-2 appear to be different. While Aβ or β-amyloid peptide is the main substrate of BACE, BACE-2 does not generate either form of Aβ (Vassar, R., Bennett, B. D., Babu-Khan, S., Kahn, S., Mendiaz, E. A., Denis, P., Teplow, D. B., Ross, S., Amarante, P., Loeloff, R., Luo, Y., Fisher, S., Fuller, J., Edenson, S., Lile, J., Jarosinski, M. A., Biere, A. L., Curran, E., Burgess, T., Louis, J.-C., Collins, F., Treanor, J., Rogers, G., and Citron, M. (1999) Science 286, 735-741).

Transmembrane protein 27 (TMEM27 or collectrin) plays an important role in β-cell proliferation and insulin secretion (Pinar Akpinar, Satoru Kuwajima, Jan Kru” tzfeldt, and Markus Stoffel (2005) Tmem27: Cell Metabolism. 2(6) 385-397) and has been identified as a substrate for BACE-2 (WO 2010/063718). Tmem27 exists as a dimer and the extracellular domain is cleaved and shed from the plasma in a β cell-specific manner. Overexpression of full-length Tmem27, but not the truncated or soluble protein increases β cell proliferation suggesting that the full length protein is required for this biological function. Tcf1 (hepatocyte nuclear factor-1α, HNF-1α) controls the transcription of TMEM27. Mice with targeted deletion of Tcf1 exhibit decreased β cell mass, and knockdown of Tmem27 using RNAi results in a reduction of cell proliferation. Transgenic mice with increased expression of Tmem27 in pancreatic β cells exhibit increased β cell mass compared to their wild-type littermates. This data indicates that TMEM27 plays a in control of β cell mass and that inhibition of BACE-2 which cleaves TMEM27 could be useful for treating loss of β cell mass and function, the underlying cause of diabetes.

Taken together, these findings suggest that the inhibition of BACE-2 may be a favourable therapeutic strategy for the treatment and prevention of metabolic disorders related to decreased β cell mass and/or function, such as type 2 diabetes.

### DESCRIPTION OF THE INVENTION

In one aspect, the present invention relates to a method of treating metabolic disorders related to decreased β cell mass and/or function comprising administering to a subject in need thereof a BACE inhibitor.

In another aspect, the present invention relates to the use of a BACE inhibitor in the manufacture of a medicament for the treatment of a metbabolic disorder related to decreased β cell mass and/or function.

In another aspect, the present invention relates to a method of treating diseases associated with inhibition of BACE-2 activity comprising administering to a subject a therapeutically effective amount of a compound of formula (I):

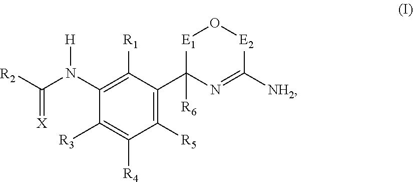

or a pharmaceutically acceptable salt thereof, wherein:
X is O or S;
R₁ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
R₂ is an aryl, heteroaryl or non-aromatic heterocyclyl group G₁, which group G₁ is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3 or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
either
R₄ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl; and
R₅ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ is hydrogen; (C₁₋₈)alkyl; halogen-(C₁₋₈)alkyl; hydroxy-(C₁₋₈)alkyl; (C₁₋₈)alkoxy-(C₁₋₈)alkyl; mercapto-(C₁₋₈)alkyl; (C₁₋₈)alkylthio-(C₁₋₈)alkyl; amino-(C₁₋₈)alkyl; N—(C₁₋₈)alkylamino-(C₁₋₈)alkyl; N,N-di-[(C₁₋₈)alkyl]amino-(C₁₋₈)alkyl with two identical or different (C₁₋₈)alkyl moieties in the N,N-di-[(C₁₋₈)alkyl]amino moiety; (C₂₋₈)alkenyl; or (C₂₋₈)alkynyl;
E₁ is —C(R₇)(R₈)—; or —C(R₇)(R₈)—C(R₉)(R₁₀)—;
E₂ is —C(R₁₁)(R₁₂)—; or —C(R₁₁)(R₁₂)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ and R₁₂ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ and R₁₂, taken together, are oxo or —CH₂—CH₂—; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—.

In another aspect, the present invention relates to a method of treating diseases associated with inhibition of BACE-2 activity comprising administering to a subject a therapeutically effective amount of a compound of the formula (Ia):

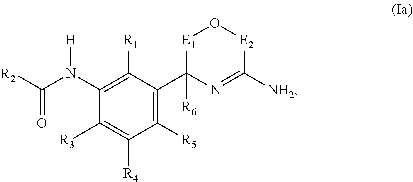

or a pharmaceutically acceptable salt thereof, wherein:
R₁ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
R₂ is a (C₃₋₈)cycloalkyl, aryl, heteroaryl or non-aromatic heterocyclyl group G₁, which group G₁ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₂, which group G₂ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
either
R₄ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl; and
R₅ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ is hydrogen; (C₁₋₈)alkyl; halogen-(C₁₋₈)alkyl; hydroxy-(C₁₋₈)alkyl; (C₁₋₈)alkoxy-(C₁₋₈)alkyl; mercapto-(C₁₋₈)alkyl; (C₁₋₈)alkylthio-(C₁₋₈)alkyl; amino-(C₁₋₈)alkyl; N—(C₁₋₈)alkylamino-(C₁₋₈)alkyl; N,N-di-[(C₁₋₈)alkyl]amino-(C₁₋₈)alkyl with two identical or different (C₁₋₈)alkyl moieties in the N,N-di-[(C₁₋₈)alkyl]amino moiety; (C₂₋₈)alkenyl; or (C₂₋₈)alkynyl;
E₁ is —C(R₇)(R₈)—; or —C(R₇)(R₈)—C(R₉)(R₁₀)—;
E₂ is —C(R₁₁)(R₁₂)—; or —C(R₁₁)(R₁₂)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ and R₁₂ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ and R₁₂, taken together, are oxo or —CH₂—CH₂—; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—.

In another aspect, the present invention relates to a method of treating diseases associated with inhibition of BACE-2 activity comprising administering to a subject a therapeutically effective amount of a compound of the formula (I):

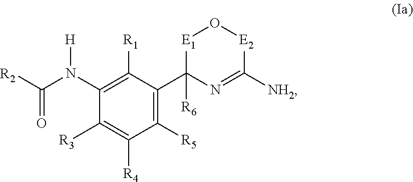

or a pharmaceutically acceptable salt thereof, wherein:
R₁ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
R₂ is a (C₃₋₈)cycloalkyl, aryl, heteroaryl or non-aromatic heterocyclyl group G₁, which group G₁ is optionally substituted by 1 to 4 substituents independently selected from the group, consisting of cyano, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₂, which group G₂ is optionally substituted by 1 to 4 substituents independently selected from the group, consisting of cyano, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
either
R₄ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl; and
R₅ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ is hydrogen; (C₁₋₈)alkyl; halogen-(C₁₋₈)alkyl; hydroxy-(C₁₋₈)alkyl; (C₁₋₈)alkoxy-(C₁₋₈)alkyl; mercapto-(C₁₋₈)alkyl; (C₁₋₈)alkylthio-(C₁₋₈)alkyl; amino-(C₁₋₈)alkyl; N—(C₁₋₈)alkylamino-(C₁₋₈)alkyl; N,N-di-[(C₁₋₈)alkyl]amino-(C₁₋₈)alkyl with two identical or different (C₁₋₈)alkyl moieties in the N,N-di-[(C₁₋₈)alkyl]amino moiety; (C₂₋₈)alkenyl; or (C₂₋₈)alkynyl;
E₁ is —C(R₇)(R₈)—; or —C(R₇)(R₈)—C(R₉)(R₁₀)—;
E₂ is —C(R₁₁)(R₁₂)—; or —C(R₁₁)(R₁₂)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ and R₁₂ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ and R₁₂, taken together, are oxo or —CH₂—CH₂—; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—.

In another aspect, the present invention relates to a method of treating diseases associated with inhibition of BACE-2 activity comprising administering to a subject a therapeutically effective amount of a compound of the formula (II):

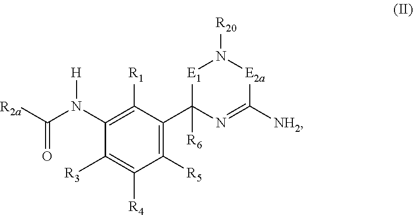

or a pharmaceutically acceptable salt thereof, wherein:
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₂ₐ is an aryl, heteroaryl or non-aromatic heterocyclyl group G₁, which group G₁ is optionally substituted by 1 to 4 substituents independently selected from the group, consisting of cyano, amino, aminocarbonyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
either
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl; and
R₅ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₈)alkylamino-(C₁₋₈)alkyl, N,N-di-[(C₁₋₈)alkyl]amino-(C₁₋₈)alkyl with two identical or different (C₁₋₈)alkyl moieties in the N,N-di-[(C₁₋₈)alkyl]amino moiety, (C₂₋₈)alkenyl, or (C₂₋₈)alkynyl;
R₂₀ is hydrogen, (C₁₋₈)alkyl, (C₁₋₈)alkyl substituted by halogen, (C₃₋₈)cycloalkyl-(C₁₋₈)alkyl, (C₃₋₈)cycloalkoxy-(C₁₋₈)alkyl, aryloxy-(C₁₋₈)alkyl, (C₁₋₈)alkoxy-(C₁₋₈)alkyl, (C₁₋₈)alkylthio-(C₁₋₈)alkyl, (C₁₋₈)alkylsulfinyl, (C₁₋₈)alkylsulfinyl-(C₁₋₈)alkyl, (C₁₋₈)alkylsulfonyl, (C₁₋₈)alkylsulfonyl-(C₁₋₈)alkyl, amino-(C₁₋₈)alkyl, (C₁₋₈)alkylamino-(C₁₋₈)alkyl, di(C₁₋₈)alkylamino-(C₁₋₈)alkyl with two identical or different (C₁₋₈)alkyl moieties in the di(C₁₋₈)alkylamino moiety, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyl-(C₁₋₈)alkyl, (C₁₋₈)alkylcarbonyl-(C₁₋₈)alkyl, (C₁₋₈)alkoxycarbonyl, halogen-(C₁₋₈)alkoxycarbonyl, (C₁₋₈)alkoxycarbonyl-(C₁₋₈)alkyl, (C₁₋₈)alkoxy-(C₁₋₈)alkylcarbonyl, or a (C₃₋₈)cycloalkylcarbonyl, arylcarbonyl, aryl-(C₁₋₈)alkylcarbonyl, heteroarylcarbonyl, heteroaryl-(C₁₋₈)alkylcarbonyl, non-aromatic heterocyclylcarbonyl, (C₃₋₈)cycloalkylsulfonyl, arylsulfonyl, aryl-(C₁₋₈)alkylsulfonyl, heteroarylsulfonyl, heteroaryl-(C₁₋₈)alkylsulfonyl, non-aromatic heterocyclylsulfonyl, (C₃₋₈)cycloalkyl, aryl, aryl-(C₁₋₈)heteroaryl, heteroaryl-(C₁₋₈)alkyl or non-aromatic heterocyclyl group G₃, which group G₃ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₄, which group G₄ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
E₁ is —C(R₇)(R₈)—, or —C(R₇)(R₈)—C(R₆)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₆R₁₇—CR₁₈R₁₉—
wherein R₁₆, R₁₇, R₁₈ and R₁₉ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—.

In another aspect, the present invention relates to a method of treating diseases associated with inhibition of BACE-2 activity comprising administering to a subject a therapeutically effective amount of a compound of the formula (II):

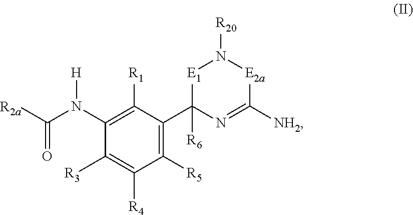

or a pharmaceutically acceptable salt thereof, wherein:
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₂ₐ is a (C₃₋₈)cycloalkyl, aryl, heteroaryl or non-aromatic heterocyclyl group G₁, which group G₁ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₂, which group G₂ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
either
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl; and
R₅ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₈)alkylamino-(C₁₋₈)alkyl, N,N-di-[(C₁₋₈)alkyl]amino-(C₁₋₈)alkyl with two identical or different (C₁₋₈)alkyl moieties in the N,N-di-[(C₁₋₈)alkyl]amino moiety, (C₂₋₈)alkenyl, or (C₂₋₈)alkynyl;

R₂₀ is hydrogen, (C₁₋₈)alkyl, (C₁₋₈)alkyl substituted by halogen, (C₃₋₈)cycloalkyl-(C₁₋₈)alkyl, (C₃₋₈)cycloalkoxy-(C₁₋₈)alkyl, aryloxy-(C₁₋₈)alkyl, (C₁₋₈)alkoxy-(C₁₋₈)alkyl, (C₁₋₈)alkylthio-(C₁₋₈)alkyl, (C₁₋₈)alkylsulfinyl, (C₁₋₈)alkylsulfinyl-(C₁₋₈)alkyl, (C₁₋₈)alkylsulfonyl, (C₁₋₈)alkylsulfonyl-(C₁₋₈)alkyl, amino-(C₁₋₈)alkyl, (C₁₋₈)alkylamino-(C₁₋₈)alkyl, di(C₁₋₈)alkylamino-(C₁₋₈)alkyl with two identical or different (C₁₋₈)alkyl moieties in the di(C₁₋₈)alkylamino moiety, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyl-(C₁₋₈)alkyl, (C₁₋₈)alkylcarbonyl-(C₁₋₈)alkyl, (C₁₋₈)alkoxycarbonyl, (C₁₋₈)alkoxycarbonyl-(C₁₋₈)alkyl, or a (C₃₋₈)cycloalkylcarbonyl, arylcarbonyl, aryl-(C₁₋₈)alkylcarbonyl, heteroarylcarbonyl, heteroaryl-(C₁₋₈)alkylcarbonyl, non-aromatic heterocyclylcarbonyl, (C₃₋₈)cycloalkylsulfonyl, arylsulfonyl, aryl-(C₁₋₈)alkylsulfonyl, heteroarylsulfonyl, heteroaryl-(C₁₋₈)alkylsulfonyl, non-aromatic heterocyclylsulfonyl, (C₃₋₈)cycloalkyl, aryl, aryl-(C₁₋₈)alkyl, heteroaryl, heteroaryl-(C₁₋₈)alkyl or non-aromatic heterocyclyl group G₃, which group G₃ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₄, which group G₄ is optionally substituted by 1 to 4 substituents independently selected from the group, consisting of cyano, aminocarbonyl, halogen, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CH₂—CH₂—; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—.

In another aspect, the invention relates to a compound of the formula (III)

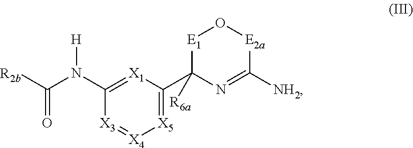

in which
either
X₁ is CR₁ or N;
X₃ is CR₃ or N;
X₄ is CR₄ or N;
X₅ is CR₅ₐ or N;
wherein at least one of X₁, X₃, X₄ and X₅ is N and not more than 2 of X₁, X₃, X₄ and X₅ are N;
or
X₁ is CR₁ or N;
X₃ is CR₃, N or S;
X₄ is a bond;
X₅ is CR₅ₐ, N or S;
wherein at least one of X₁, X₃ and X₅ is N or S, not more than 2 of X₁, X₃ and X₅ are N and not more than 1 of X₃ and X₅ are S;
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R_{2b} is an aryl, heteroaryl or non-aromatic heterocyclyl group G₁, which group G₁ is optionally substituted by 1, 2, 3 or 4 substituents independently selected from the group, consisting of cyano, amino, amino-(C₁₋₈)alkyl, N—(C₁₋₄)alkyl-amino-(C₁₋₈)alkyl, N,N-di(C₁₋₄)alkyl-amino-(C₁₋₈)alkyl, aminocarbonyl, thiocarbamoyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₃₋₈)cycloalkyl-(C₁₋₈)alkoxy, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3, or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₅ₐ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅ₐ, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ₐ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₄)alkyl-amino-(C₁₋₈)alkyl, N,N-di(C₁₋₄)alkyl-amino-(C₁₋₈)alkyl, (C₂₋₈)alkenyl, or (C₂₋₈)alkynyl;
or
R₅ₐ and R₆ₐ taken together, are a (C₁₋₄)alkylene group, in which (C₁₋₄)alkylene group 1 —CH₂— ring member is optionally replaced with a hetero ring member independently selected from the group, consisting of —N(H)—, —N[(C₁₋₄)alkyl]-, —O—, —S—, —S(═O)— or —S(═O)₂—;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₅R₁₆—CR₁₇R₁₈—
wherein R₁₅, R₁₆, R₁₇ and R₁₈ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—;
   or a pharmaceutically acceptable salt thereof.

Halogen denotes fluorine, chlorine, bromine or iodine.

A halogenated group or moiety, such as halogenalkyl, can be mono-, poly- or per-halogenated.

An aryl group, ring or moiety is a naphthyl or, preferably, phenyl group, ring or moiety.

A heteroaryl group, ring or moiety is a monocyclic aromatic 5- or 6-membered structure, in which structure 1, 2, 3 or 4 ring members are hetero ring members independently selected from the group, consisting of a nitrogen ring member, an oxygen ring member and a sulfur ring member, such as furyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl or pyridyl; or

a bicyclic aromatic 9- or 10- or membered structure, in which structure 1, 2, 3, 4 or 5 ring members are hetero ring members independently selected from the group, consisting of a nitrogen ring member, an oxygen ring member and a sulfur ring member. The fused rings completing the bicyclic groups may contain only carbon atoms and may be saturated, partially saturated, or unsaturated. Heteroaryl groups which are bicyclic include at least one fully aromatic ring but the other fused ring may be aromatic or non-aromatic. Examples of bicyclic heteroaryl groups include, benzofuranyl, benzothiophenyl, imidazopyridinyl, indazolyl, indolyl, isoquinolinyl, pyrazolopyridinyl and quinolinyl. The heteroaryl radical may be bonded via a carbon atom or heteroatom.

In one embodiment, the heteroaryl group is an aromatic 5- or 6-membered structure, in which structure 1, 2, 3 or 4 ring members are hetero ring members independently selected from the group, consisting of a nitrogen ring member, an oxygen ring member and a sulfur ring member.

A non-aromatic heterocyclyl group, ring or moiety is a non-aromatic 4-, 5-, 6- or 7-membered cyclic structure, in which cyclic structure 1, 2 or 3 ring members are hetero ring members independently selected from the group, consisting of a nitrogen ring member, an oxygen ring member and a sulfur ring member, such as azetidinyl, oxetanyl, pyrrolinyl, pyrrolidyl, tetrahydrofuryl, tetrahydrothienyl, piperidyl, piperazinyl, tetrahydropyranyl, morpholinyl or perhydroazepinyl.

Any non-cyclic carbon containing group or moiety with more than 1 carbon atom is straight-chain or branched.

Unless defined otherwise, carbon containing groups, moieties or molecules contain 1 to 8, 1 to 6, 1 to 4 or 1 or 2 carbon atoms.

The terms “alkoxy”, “alkenoxy” and “alkynoxy” respectively denote alkyl, alkenyl and alkynyl groups when linked by oxygen.

On account of one or more than one asymmetrical carbon atom, which may be present in a compound of the formula (I), (Ia), and (II), a corresponding compound of the formula (I), (Ia) and (II), respectively, may exist in pure optically active form or in the form of a mixture of optical isomers, e.g. in the form of a racemic mixture. All of such pure optical isomers and all of their mixtures, including the racemic mixtures, are part of the present invention.

In one embodiment, the invention therefore relates to a method of treating diseases associated with inhibition of BACE-2 activity comprising administering to a subject a therapeutically effective amount of a compound of the formula (I′)

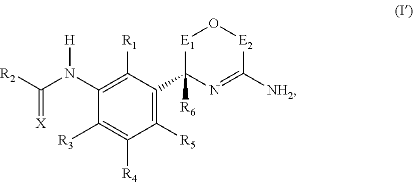

or a pharmaceutically acceptable salt thereof, wherein:
X, E₁, E₂, R₁, R₂, R₃, R₄, R₅ and R₆ are as defined hereinbefore in relation to the formula I.

In another embodiment, the invention therefore relates to a method of treating diseases associated with inhibition of BACE-2 activity comprising administering to a subject a therapeutically effective amount of a compound of the formula (I″):

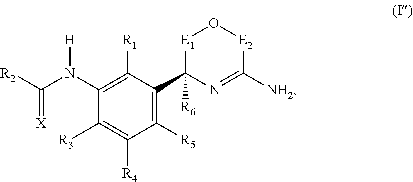

or a pharmaceutically acceptable salt thereof, wherein:
X, E₁, E₂, R₁, R₂, R₃, R₄, R₅ and R₆ are as defined hereinbefore in relation to the formula I.

In another embodiment, the invention therefore relates to a method of treating diseases associated with inhibition of BACE-2 activity comprising administering to a subject a therapeutically effective amount of a compound of the formula (Ia″):

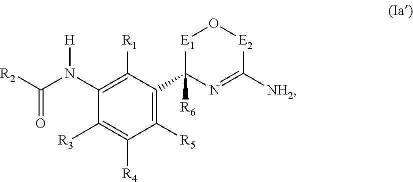

or a pharmaceutically acceptable salt thereof, wherein:
E₁, E₂, R₁, R₂, R₃, R₄, R₅ and R₆ are as defined hereinbefore in relation to the formula (Ia).

In another embodiment, the invention therefore relates to a method of treating diseases associated with inhibition of BACE-2 activity comprising administering to a subject a therapeutically effective amount of a compound of the formula (Ia″):

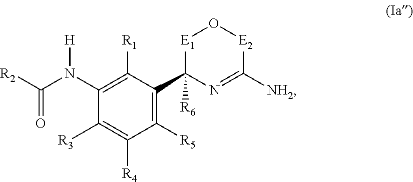

or a pharmaceutically acceptable salt thereof, wherein:
E₁, E₂, R₁, R₂, R₃, R₄, R₅ and R₆ are as defined hereinbefore in relation to the formula (Ia).

In another embodiment, the invention therefore relates to a method of treating diseases associated with inhibition of BACE-2 activity comprising administering to a subject a therapeutically effective amount of a compound of the formula (II′):

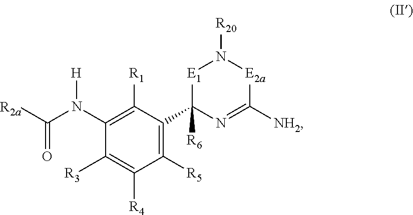

or a pharmaceutically acceptable salt thereof, wherein:
E₁, E₂ₐ, R₁, R₂ₐ, R₃, R₄, R₅, R₆ and R₂₀ are as defined hereinbefore in relation to the formula (II).

In another embodiment, the invention therefore relates to a method of treating diseases associated with inhibition of BACE-2 activity comprising administering to a subject a therapeutically effective amount of a compound of the formula (II″):

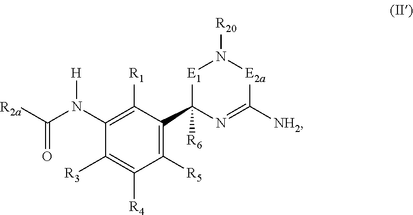

or a pharmaceutically acceptable salt thereof, wherein:
E₁, E₂ₐ, R₁, R₂ₐ, R₃, R₄, R₅, R₆ and R₂₀ are as defined hereinbefore in relation to the formula (II).

In one embodiment, the invention therefore relates to a compound of the formula (III′)

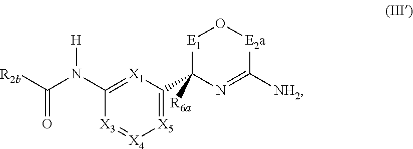

in which
E₁, E₂₂, R_{2b}, R₆, X₁, X₃, X₄ and X₅ are as defined hereinbefore in relation to the formula (III),
   or a pharmaceutically acceptable salt thereof.

In one embodiment, the invention therefore relates to a compound of the formula (III″)

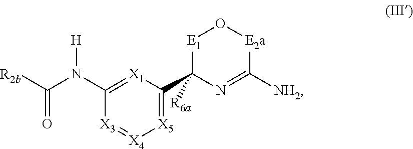

in which
E₁, E₂ₐ, R_{2b}, R₆, X₁, X₃, X₄ and X₅ are as defined hereinbefore in relation to the formula (III),
   or a pharmaceutically acceptable salt thereof.

The phrase “a compound of the invention” refers to a compound of formula (I), (I′), (I″), (Ia), (Ia′), (Ia″), (Ib), (Ib′), (II), (II′), (II″), (III), (III′), or (III″), or any embodiment thereof including the examples.

As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers or axes and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration.

A compound of the invention may exist in tautomeric form. All such tautomers are part of the present invention.

A compound of the invention may exist in free form or in salt form, for example a basic compound in acid addition salt form or an acidic compound in the form of a salt with a base. All of such free compounds and salts are part of the present invention.

In one embodiment, the invention relates to a compound of the invention, as defined herein, in free form. In another embodiment, the invention relates to a compound of the invention, as defined herein, in salt form. In a further embodiment, the invention relates to a compound of the invention, as defined herein, in pharmaceutically acceptable salt form. In yet a further embodiment, the invention relates to a compound of the invention, as defined herein, in hydrochloride salt form. In yet a further embodiment, the invention relates to any one of the compounds of the Examples in free form. In yet a further embodiment, the invention relates to any one of the compounds of the Examples in pharmaceutically acceptable salt form. In yet a further embodiment, the invention relates to any one of the compounds of the Examples in hydrochloride salt form.

As used herein, the terms “salt” or “salts” refers to an acid addition or base addition salt of a compound of the invention. “Salts” include in particular “pharmaceutical acceptable salts”. The term “pharmaceutically acceptable salts” refers to salts that retain the biological effectiveness and properties of a compound of the invention and, which typically is not biologically or otherwise undesirable. In many cases, a compound of the present invention is capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts. Inorganic acids from which salts can be derived include, for example, hydrochloric acid, hydrobromic acid, sulfuric acid, nitric acid and phosphoric acid. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid and sulfosalicylic acid. Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines and basic ion exchange resins. Certain organic amines include isopropylamine, benzathine, cholinate, diethanolamine, diethylamine, lysine, meglumine, piperazine and tromethamine.

The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

When both a basic group and an acid group are present in the same molecule, a compound of the invention may also form internal salts, e.g., zwitterionic molecules.

The present invention also provides pro-drugs of compounds of the invention that convert in vivo to a compound of the present invention. A pro-drug is an active or inactive compound that is modified chemically through in vivo physiological action, such as hydrolysis, metabolism and the like, into a compound of this invention following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001). Generally, bioprecursor prodrugs are compounds, which are inactive or have low activity compared to the corresponding active drug compound, that contain one or more protective groups and are converted to an active form by metabolism or solvolysis. Both the active drug form and any released metabolic products should have acceptably low toxicity.

Carrier prodrugs are drug compounds that contain a transport moiety, e.g., that improve uptake and/or localized delivery to a site(s) of action. Desirably for such a carrier prodrug, the linkage between the drug moiety and the transport moiety is a covalent bond, the prodrug is inactive or less active than the drug compound, and any released transport moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of (a) hydroxyl groups with lipophilic carboxylic acids (e.g., a carboxylic acid having at least one lipophilic moiety), or (b) carboxylic acid groups with lipophilic alcohols (e.g., an alcohol having at least one lipophilic moiety, for example aliphatic alcohols).

Exemplary prodrugs are, e.g., esters of free carboxylic acids and S-acyl derivatives of thiols and O-acyl derivatives of alcohols or phenols, wherein acyl has a meaning as defined herein. Suitable prodrugs are often pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxymethyl ester and the like conventionally used in the art. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

Furthermore, a compound of the invention, or a salt thereof, can also be obtained in the form of a hydrate, or include other solvents used for their crystallization. A compound of the invention may inherently or by design form a solvate with pharmaceutically acceptable solvent (including water); therefore, it is intended that the invention embrace both solvated and unsolvated forms. The term “solvate” refers to a molecular complex of a compound of the invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

A compound of the invention, or a pharmaceutically acceptable salt, hydrate or solvate thereof, may inherently or by design form polymorphs. In one embodiment, the invention therefore relates to a compound of the invention, as defined herein, or a pharmaceutically acceptable salt thereof, in crystalline form.

The present invention includes all pharmaceutically acceptable isotope-labeled compounds of the invention, wherein one or more than one atom is/are replaced by one or more than one atom having the same atomic number as, but an atomic mass different from, the one(s) usually found in nature. Examples of such isotopes are those of carbon, such as ¹¹C, ¹³C or ¹⁴C, chlorine, such as ³⁶Cl, fluorine, such as ¹⁸F, bromine, such as ⁷⁶Br, hydrogen, such as ²H or ³H, iodine, such as ¹²³I, ¹²⁴I, ¹²⁵I or ¹³¹I, nitrogen, such as ¹³N or ¹⁵N, oxygen, such as ¹⁵O, ¹⁷O or ¹⁸O, phosphorus, such as ³²P, or sulphur, such as ³⁵S. An isotope-labeled compound of the invention can be prepared by a process analogous to those described in the Examples or by a conventional technique known to those skilled in the art using an appropriate isotopically-labeled reagent or starting material. The incorporation of a heavier isotope, such as ²H (D), may provide greater metabolic stability to a compound of the invention, which may result in, for example, an increased in vivo-half-life of the compound or in reduced dosage requirements. Certain isotope-labeled compounds of the invention, for example those incorporating a radioactive isotope, such as ³H or ¹⁴C, may be used in drug or substrate-tissue distribution studies. A compounds of the invention with a positron emitting isotope, such as ¹¹C, ¹⁸F, ¹³N or ¹⁵O, may be useful in positron emission tomography (PET) or single photon emission computed tomography (SPECT) studies, e.g. to examine substrate-receptor occupancies.

Pharmaceutically acceptable solvates in accordance with the invention include those wherein the solvent of crystallization may be isotopically substituted, e.g. D₂O, d6-acetone, d6-DMSO.

A compound of the invention that contains a group capable of acting as a donor and/or acceptor for hydrogen bonds may be capable of forming co-crystals with suitable co-crystal formers. These co-crystals may be prepared from a compounds of the invention by known co-crystal forming procedures. Such procedures include grinding, heating, co-subliming, co-melting, or contacting in solution a compounds of the invention with the co-crystal former under crystallization conditions and isolating co-crystals thereby formed. Suitable co-crystal formers include those described in WO 2004/078163. Hence the invention further provides co-crystals comprising a compound of the invention.

In certain embodiments, the invention relates to a method of treating diseases associated with inhibition of BACE-2 activity comprising administering to a subject a therapeutically effective amount of a compound of the formula (I), (I′), (I″), (Ia), (Ia′), or (Ia″), or a pharmaceutically acceptable salt thereof, wherein:

(1) R₁ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
(2) R₁ is hydrogen;
(3) R₂ is an aryl, heteroaryl or non-aromatic heterocyclyl group G₁, which group G₁ is optionally substituted by 1, 2, 3 or 4 substituents independently selected from the group, consisting of cyano, nitro, amino, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3 or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
(4) R₂ is an aryl, heteroaryl or non-aromatic heterocyclyl group G₁, which group G₁ is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3 or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
(5) R₂ is an aryl or heteroaryl group G₁, which group G₁ is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)-alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3 or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
(6) R₂ is a (C₃₋₈)cycloalkyl, aryl, heteroaryl or non-aromatic heterocyclyl group G₁, which group G₁ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₂, which group G₂ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
(7) R₂ is a (C₃₋₈)cycloalkyl, aryl, heteroaryl or non-aromatic heterocyclyl group G₁, which group G₁ is optionally substituted by 1 to 4 substituents independently selected from the group, consisting of cyano, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₂, which group G₂ is optionally substituted by 1 to 4 substituents independently selected from the group, consisting of cyano, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)-alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
(8) R₂ is a (C₃₋₈)cycloalkyl, aryl or heteroaryl group G₁, which group G₁ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl or heteroaryl group G₂, which group G₂ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
(9) R₂ is a heteroaryl group G₁, which group G₁ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl or heteroaryl group G₂, which group G₂ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
(10) R₂ is a heteroaryl group G₁, which group G₁ is optionally substituted by 1 or 2 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl or heteroaryl group G₂, which group G₂ is unsubstituted;
(11) R₂ is a heteroaryl or aryl group which is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy and (C₂₋₈)alkynoxy;

(12) R₂ is a heteroaryl group which is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy and (C₂₋₈)alkynoxy;

(13) R₂ is a heteroaryl group which is optionally substituted by 1, 2, 3 or 4 substituents independently selected from the group, consisting of deuterium, cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, deuterated (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy and (C₂₋₈)alkynoxy;
(14) R₂ is a heteroaryl group which contains 1, 2 or 3 nitrogen atom ring members and is optionally substituted by 1, 2, 3 or 4 substituents independently selected from the group, consisting of deuterium, cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, deuterated (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy and (C₂₋₈)alkynoxy;
(15) R₂ is a monocyclic 6-membered heteroaryl group which contains 1, 2 or 3 nitrogen atom ring members and which is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy and (C₂₋₈)alkynoxy;
(16) R₂ is a 6-membered heteroaryl group which contains 1, 2 or 3 nitrogen atom ring members and which is substituted by 1, 2, 3 or 4 substituents and wherein one of the substituents is located at the para position of the heteroaryl group relative to the amide linker and wherein the substituents are independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxyl-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy and (C₂₋₈)alkynoxy;
(17) R₂ is a 6-membered heteroaryl group which contains 1, 2 or 3 nitrogen atom ring members and which is substituted by 1, 2, 3 or 4 substituents and wherein one of the substituents is located at the para position of the heteroaryl group relative to the amide linker and wherein the substituents are independently selected from the group, consisting of cyano, halogen (C₁₋₆)alkyl, (C₁₋₆)alkoxy, (C₁₋₆)alkoxy-(C₁₋₆)alkoxy, halogen-(C₁₋₆)alkyl and (C₂₋₈)alkynoxy;
(18) R₂ is a 6-membered heteroaryl group which contains 1, 2 or 3 nitrogen atom ring members and which is optionally substituted by 1, 2, 3 or 4 substituents independently selected from the group, consisting of cyano, halogen (C₁₋₆)alkyl, (C₁₋₆)alkoxy, (C₁₋₆)alkoxy-(C₁₋₆)alkoxy, halogen-(C₁₋₆)alkyl and (C₂₋₈)alkynoxy;
(19) R₂ is a pyridyl or pyrazinyl group which is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3 or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
(20) R₂ is a pyridyl or pyrazinyl group which is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl or heteroaryl group G₂, which group G₂ is unsubstituted;
(21) R₂ is a pyridyl or pyrazinyl group which is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy and (C₂₋₈)alkynoxy;
(22) R₂ is a pyridyl or pyrazinyl group which is optionally substituted by 1, 2, 3 or 4 substituents independently selected from the group, consisting of cyano, halogen (C₁₋₆)alkyl, (C₁₋₆)alkoxy, (C₁₋₆)alkoxy-(C₁₋₆)alkoxy, halogen-(C₁₋₆)alkyl and (C₂₋₈)alkynoxy;
(23) R₂ is a pyridyl or pyrazinyl group which is optionally substituted by 1, 2, 3 or 4 substituents independently selected from the group, consisting of deuterium, cyano, halogen, (C₁₋₆)alkyl, deuterated (C₁₋₆)alkyl, (C₁₋₆)alkoxy, (C₁₋₆)alkoxy-(C₁₋₆)alkoxy, halogen-(C₁₋₆)alkyl and (C₂₋₈)alkynoxy;
(24) R₂ is a pyridyl group which is optionally substituted by 1, 2, 3 or 4 substituents independently selected from the group, consisting of cyano, halogen (C₁₋₆)alkyl, (C₁₋₆)alkoxy, (C₁₋₆)alkoxy-(C₁₋₆)alkoxy, halogen-(C₁₋₆)alkyl and (C₂₋₈)alkynoxy;
(25) R₂ is a pyrazinyl group which is optionally substituted by 1, 2, 3 or 4 substituents independently selected from the group, consisting of cyano, halogen (C₁₋₆)alkyl, (C₁₋₆)alkoxy, (C₁₋₆)alkoxy-(C₁₋₆)alkoxy, halogen-(C₁₋₆)alkyl and (C₂₋₈)alkynoxy;
(26) R₂ is a pyridyl or pyrazinyl group which is substituted by 1, 2, 3 or 4 substituents and wherein one of the substituents is located at the para position of the pyridyl or pyrazinyl group relative to the amide linker and wherein the substituents are independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy and (C₂₋₈)alkynoxy;
(27) R₂ is a pyridyl or pyrazinyl group which is substituted by 1, 2, 3 or 4 substituents and wherein one of the substituents is located at the para position of the pyridyl or pyrazinyl group relative to the amide linker and wherein the substituents are independently selected from the group, consisting of deuterium, cyano, halogen, (C₁₋₆)alkyl, deuterated (C₁₋₆)alkyl, (C₁₋₆)alkoxy, (C₁₋₆)alkoxy-(C₁₋₆)alkoxy, halogen-(C₁₋₆)alkyl and (C₂₋₆)alkynoxy;
(28) R₂ is a pyridyl or pyrazinyl group which is substituted by 1, 2, 3 or 4 substituents and wherein one of the substituents is located at the para position of the pyridyl or pyrazinyl group relative to the amide linker and wherein the substituents are independently selected from the group, consisting of cyano, halogen (C₁₋₆)alkyl, (C₁₋₆)alkoxy, (C₁₋₆)alkoxy-(C₁₋₆)alkoxy, halogen-(C₁₋₆)alkyl and (C₂₋₆)alkynoxy;
(29) R₂ is a pyridyl or pyrazinyl group which is substituted by 2, 3 or 4 substituents and wherein one of the substituents is located at the para position and one of the substituents is located at the ortho position of the pyridyl or pyrazinyl group relative to the amide linker and wherein the substituents are independently selected from the group, consisting of cyano, halogen (C₁₋₆)alkyl, (C₁₋₆)alkoxy, (C₁₋₆)alkoxy-(C₁₋₆)alkoxy, halogen-(C₁₋₆)alkyl and (C₂₋₆)alkynoxy;
(30) R₂ is a pyridyl or pyrazinyl group which is substituted by 2 substituents and wherein one of the substituents is located at the para position and one of the substituents is located at the ortho position of the pyridyl or pyrazinyl group relative to the amide linker and wherein the substituents are independently selected from the group, consisting of cyano, halogen (C₁₋₆)alkyl, (C₁₋₆)alkoxy, (C₁₋₆)alkoxy-(C₁₋₆)alkoxy, halogen-(C₁₋₆)alkyl and (C₂₋₆)alkynoxy;
(31) R₂ is a pyridyl or pyrazinyl group which is substituted by 2 substituents and wherein one of the substituents is located at the para position and one of the substituents is located at the ortho position of the pyridyl or pyrazinyl group relative to the amide linker and wherein the substituents are independently selected from the group, consisting of deuterium, cyano, chloro, bromo, (C₁₋₆)alkyl, deuterated (C₁₋₆)alkyl, (C₁₋₆)alkoxy, (C₁₋₃)alkoxy-(C₁₋₃)alkoxy, trifluoromethyl and (C₂₋₄)alkynoxy;
(32) R₂ is a pyridyl or pyrazinyl group which is substituted by 2 substituents and wherein one of the substituents is located at the para position and one of the substituents is located at the ortho position of the pyridyl or pyrazinyl group relative to the amide linker and wherein the substituents are independently selected from the group, consisting of cyano, chloro, bromo, (C₁₋₆)alkyl, (C₁₋₆)alkoxy, (C₁₋₃)alkoxy-(C₁₋₃)alkoxy, trifluoromethyl and (C₂₋₄)alkynoxy;
(33) R₃ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
(34) R₃ is hydrogen;
(35) either
R₄ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl; and
R₅ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
(36) R₄ is hydrogen; or halogen; and
R₅ is hydrogen; or halogen;
(37) R₄ is hydrogen; and
R₅ is halogen;
(38) R₄ is halogen; and
R₅ is hydrogen;
(39) each of R₄ and R₅ is hydrogen;
(40) R₄ is hydrogen; and
R₅ is fluoro or chloro;
(41) R₆ is hydrogen; (C₁₋₈)alkyl; halogen-(C₁₋₈)alkyl; hydroxy-(C₁₋₈)alkyl; (C₁₋₈)alkoxy-(C₁₋₈)alkyl; mercapto-(C₁₋₈)alkyl; (C₁₋₈)alkylthio-(C₁₋₈)alkyl; amino-(C₁₋₈)alkyl; N—(C₁₋₈)alkylamino-(C₁₋₈)alkyl; N,N-di-[(C₁₋₈)alkyl]amino-(C₁₋₈)alkyl with two identical or different (C₁₋₈)alkyl moieties in the N,N-di-[(C₁₋₈)alkyl]amino moiety; (C₂₋₈)alkenyl; or (C₂₋₈)alkynyl;
(42) R₆ is (C₁₋₈)alkyl; or halogen-(C₁₋₈)alkyl;
(43) R₆ is (C₁₋₃)alkyl; or halogen-(C₁₋₃)alkyl;
(44) R₆ is (C₁₋₈)alkyl; or fluorine-substituted (C₁₋₈)alkyl;
(45) R₆ is (C₁₋₃)alkyl; or fluorine-substituted (C₁₋₃)alkyl;
(46) R₆ is methyl, fluoromethyl or di-fluoromethyl;
(47) R₆ is di-fluoromethyl;

### (48) E₁ is —C(R₇)(R₈)—; or —C(R₇)(R₈)—C(R₉)(R₁₀)—;

### (49) E₁ is —C(R₇)(R₈)—;

### (50) E₂ is —C(R₁₁)(R₁₂)—; or —C(R₁₁)(R₁₂)—C(R₁₃)(R₁₄)—;

### (51) E₂ is —C(R₁₁)(R₁₂)—;

(52) either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
(53) each of R₇ and R₈ is independently selected from hydrogen and fluoro;
(54) each of R₇ and R₈ is hydrogen;
(55) either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
(56) each of R₉ and R₁₀ is hydrogen;
(57) either
each of R₁₁ and R₁₂ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ and R₁₂, taken together, are oxo or —CH₂—CH₂—;
(58) each of R₁₁ and R₁₂ is independently selected from the group, consisting of hydrogen, halogen, (C₁₋₈)alkyl and halogen-(C₁₋₈)alkyl;
(59) each of R₁₁ and R₁₂ is independently selected from the group, consisting of hydrogen, (C₁₋₈)alkyl and halogen-(C₁₋₈)alkyl;
(60) R₁₁ is (C₁₋₈)alkyl, and R₁₂ is halogen-(C₁₋₈)alkyl;
(61) each of R₁₁ and R₁₂ is independently selected from the group, consisting of hydrogen, (C₁₋₃)alkyl and halogen-(C₁₋₃)alkyl;
(62) each of R₁₁ and R₁₂ is independently selected from the group, consisting of hydrogen, methyl, fluoromethyl, difluoromethyl and trifluoromethyl;
(63) each of R₁₁ and R₁₂ is independently selected from the group, consisting of hydrogen, methyl and trifluoromethyl;
(64) each of R₁₁ and R₁₂ is hydrogen;
(65) either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—.

### (66) X is O;

### (67) X is S.

The skilled person would understand that the embodiments (1) to (67) may be used independently, collectively or in any combination or sub-combination to the limit the scope of the invention as described hereinbefore in relation to compounds of the formula (I), (I′), (I″), (Ia), (Ia′), or (Ia″).

In one embodiment, the invention relates to a method of treating diseases associated with inhibition of BACE-2 activity comprising administering to a subject a therapeutically effective amount of a compound of formula (Ib):

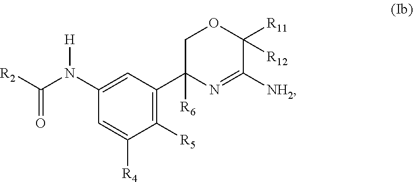

or a pharmaceutically acceptable salt thereof, wherein:
R₂ is a heteroaryl group which is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy and (C₂₋₈)alkynoxy;
R₄ is hydrogen; or halogen;
R₅ is hydrogen; or halogen;
R₆ is (C₁₋₈)alkyl; or halogen-(C₁₋₈)alkyl; and
each of R₁₁ and R₁₂ is independently selected from the group, consisting of hydrogen, methyl, fluoromethyl, difluoromethyl and trifluoromethyl.

In another embodiment, the invention relates to a method of treating diseases associated with inhibition of BACE-2 activity comprising administering to a subject a therapeutically effective amount of a compound of the formula (Ib′):

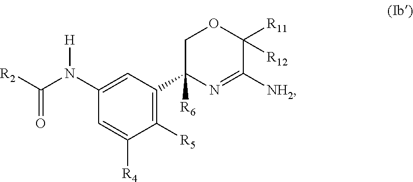

or a pharmaceutically acceptable salt thereof, wherein:
R₂ is a pyridyl or pyrazinyl group which is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy and (C₂₋₈)alkynoxy;
R₄ is hydrogen; or halogen;
R₅ is hydrogen; or halogen;
R₆ is (C₁₋₈)alkyl; or halogen-(C₁₋₈)alkyl; and
each of R₁₁ and R₁₂ is independently selected from the group, consisting of hydrogen, methyl, fluoromethyl, difluoromethyl and trifluoromethyl.

In yet another embodiment, the invention relates to a method of treating diseases associated with inhibition of BACE-2 activity comprising administering to a subject a therapeutically effective amount of a compound of formula (Ib′), or a pharmaceutically acceptable salt thereof, wherein:

R₂ is a pyridyl or pyrazinyl group which is substituted by 1, 2, 3 or 4 substituents and wherein one of the substituents is located at the para position of the pyridyl or pyrazinyl group relative to the amide linker and wherein the substituents are independently selected from the group, consisting of cyano, halogen (C₁₋₆)alkyl, (C₁₋₆)alkoxy, (C₁₋₆)alkoxy-(C₁₋₆)alkoxy, halogen-(C₁₋₆)alkyl and (C₂₋₆)alkynoxy;
R₄ is hydrogen; or halogen;
R₅ is hydrogen; or halogen;
R₆ is methyl, fluoromethyl or di-fluoromethyl; and
each of R₁₁ and R₁₂ is independently selected from the group, consisting of hydrogen, methyl, fluoromethyl, difluoromethyl and trifluoromethyl.

In particular embodiments, the invention relates to one or more than one, e.g. all, of the compounds of the invention mentioned in the Examples hereinafter, in free form or in salt form. In one embodiment, the invention relates to one compound of the invention mentioned in the Examples hereinafter, in free form. In another embodiment, the invention relates to one compound of the invention mentioned in the Examples hereinafter, in salt form. In a further embodiment, the invention relates to one compound of the invention mentioned in the Examples hereinafter, in pharmaceutically acceptable salt form. In yet a further embodiment, the invention relates to one compound of the invention mentioned in the Examples hereinafter, in hydrochloride salt form.

In another embodiment, the invention relates to a method of treating diseases associated with inhibition of BACE-2 activity comprising administering to a subject a therapeutically effective amount of a compound of the invention, or a pharmaceutically acceptable salt thereof, which is selected from:
Furan-2-carboxylic acid [3-(5-amino-3-methyl-3,6-dihydro-2H-[1,4]oxazin-3-yl)-phenyl]-amide;
5-Bromo-pyridine-2-carboxylic acid [3-(5-amino-3-methyl-3,6-dihydro-2H-[1,4]oxazin-3-yl)-phenyl]-amide;
5-Bromo-pyridine-2-carboxylic acid [3-(5-amino-3-methyl-3,6-di [symbol] hydro-2H-[1,4]oxazin-3-yl)-phenyl]-amide;
5-Bromo-pyridine-2-carboxylic acid [3-(5-amino-3-methyl-3,6-di [symbol] hydro-2H-[1,4]oxazin-3-yl)-phenyl]-amide;
2-Methyl-oxazole-4-carboxylic acid [3-(5-amino-3-methyl-3,6-dihydro-2H-[1,4]oxazin-3-yl)-phenyl]-amide;
5-Methyl-pyrazine-2-carboxylic acid [3-(5-amino-3-methyl-3,6-dihydro-2H-[1,4]oxazin-3-yl)-phenyl]-amide;
5-Bromo-pyrimidine-2-carboxylic acid [3-(5-amino-3-methyl-3,6-dihydro-2H-[1,4]oxazin-3-yl)-phenyl]-amide;
Imidazo[1,2-a]pyridine-2-carboxylic acid [3-(5-amino-3-methyl-3,6-dihydro-2H-[1,4]oxazin-3-yl)-phenyl]-amide;
3-Fluoro-pyridine-2-carboxylic acid [3-(5-amino-3-methyl-3,6-dihydro-2H-[1,4]oxazin-3-yl)-phenyl]-amide;
2,5-Dimethyl-oxazole-4-carboxylic acid [3-(5-amino-3-methyl-3,6-dihydro-2H-[1,4]oxazin-3-yl)-phenyl]-amide;
2-Methyl-thiazole-4-carboxylic acid [3-(5-amino-3-methyl-3,6-dihydro-2H-[1,4]oxazin-3-yl)-phenyl]-amide;
6-Hydroxy-pyridazine-3-carboxylic acid [3-(5-amino-3-methyl-3,6-dihydro-2H-[1,4]oxazin-3-yl)-phenyl]-amide;
Pyridine-2-carboxylic acid [3-(5-amino-3-methyl-3,6-dihydro-2H-[1,4]oxazin-3-yl)-phenyl]-amide;
5-Cyano-pyridine-2-carboxylic acid [3-(5-amino-3-methyl-3,6-dihydro-2H-[1,4]oxazin-3-yl)-phenyl]-amide;
5-(3-Trifluoromethyl-pyrazol-1-yl)-pyrazine-2-carboxylic acid [3-(5-amino-3-methyl-3,6-dihydro-2H-[1,4]oxazin-3-yl)-phenyl]-amide;
5-(3-Methyl-pyrazol-1-yl)-pyrazine-2-carboxylic acid [3-(5-amino-3-methyl-3,6-dihydro-2H-[1,4]oxazin-3-yl)-phenyl]-amide;
3-Chloro-5-trifluoromethyl-pyridine-2-carboxylic acid [3-(5-amino-3-methyl-3,6-dihydro-2H-[1,4]oxazin-3-yl)-phenyl]-amide;
5-Methoxy-pyrazine-2-carboxylic acid [3-(5-amino-3-methyl-3,6-dihydro-2H-[1,4]oxazin-3-yl)-phenyl]-amide;
5-Hydroxy-pyrazine-2-carboxylic acid [3-(5-amino-3-methyl-3,6-dihydro-2H-[1,4]oxazin-3-yl)-phenyl]-amide;
4-Bromo-furan-2-carboxylic acid [3-(5-amino-3-methyl-3,6-dihydro-2H-[1,4]oxazin-3-yl)-phenyl]-amide;
5-Chloro-pyridine-2-carboxylic acid [3-(5-amino-3-methyl-3,6-dihydro-2H-[1,4]oxazin-3-yl)-phenyl]-amide;
5-Trifluoromethyl-pyridine-2-carboxylic acid [3-(5-amino-3-methyl-3,6-dihydro-2H-[1,4]oxazin-3-yl)-phenyl]-amide;
5-Trifluoromethyl-furan-3-carboxylic acid [3-(5-amino-3-methyl-3,6-dihydro-2H-[1,4]oxazin-3-yl)-phenyl]-amide;
N-[3-(5-Amino-3-methyl-3,6-dihydro-2H-[1,4]oxazin-3-yl)-phenyl]-4-bromo-benzamide;
5-Methyl-pyridine-2-carboxylic acid [3-(5-amino-3-methyl-3,6-dihydro-2H-[1,4]oxazin-3-yl)-phenyl]-amide;
N-[3-(5-Amino-3-methyl-3,6-dihydro-2H-[1,4]oxazin-3-yl)-phenyl]-nicotinamide;
5-Bromo-3-methyl-pyridine-2-carboxylic acid [3-(5-amino-3-methyl-3,6-dihydro-2H-[1,4]oxazin-3-yl)-phenyl]-amide;
5-Methoxy-pyridine-2-carboxylic acid [3-(5-amino-3-methyl-3,6-dihydro-2H-[1,4]oxazin-3-yl)-phenyl]-amide;
5-Bromo-pyridine-2-carboxylic acid [3-(5-amino-3-fluoromethyl-3,6-dihydro-2H-[1,4]oxazin-3-yl)-phenyl]-amide;
5-Bromo-pyridine-2-carboxylic acid [3-(5-amino-3-fluoromethyl-3,6-dihydro-2H-[1,4]oxazin-3-yl)-5-chloro-phenyl]-amide;
5-Bromo-pyridine-2-carboxylic acid [3-(5-amino-3-fluoromethyl-3,6-dihydro-2H-[1,4]oxazin-3-yl)-phenyl]-amide;
5-Bromo-pyridine-2-carboxylic acid [3-(5-amino-3-difluoromethyl-3,6-dihydro-2H-[1,4]oxazin-3-yl)-phenyl]-amide;
5-Chloro-pyridine-2-carboxylic acid [3-(5-amino-3-difluoromethyl-3,6-dihydro-2H-[1,4]oxazin-3-yl)-phenyl]-amide;
5-Cyano-pyridine-2-carboxylic acid [3-(5-amino-3-difluoromethyl-3,6-dihydro-2H-[1,4]oxazin-3-yl)-phenyl]-amide;
5-Bromo-3-methyl-pyridine-2-carboxylic acid [3-(5-amino-3-difluoromethyl-3,6-dihydro-2H-[1,4]oxazin-3-yl)-phenyl]-amide;
5-Methyl-pyrazine-2-carboxylic acid [3-(5-amino-3-difluoromethyl-3,6-dihydro-2H-[1,4]oxazin-3-yl)-phenyl]-amide;
5-Trifluoromethyl-pyridine-2-carboxylic acid [3-(5-amino-3-difluoromethyl-3,6-dihydro-2H-[1,4]oxazin-3-yl)-phenyl]-amide;
3-Chloro-5-trifluoromethyl-pyridine-2-carboxylic acid [3-(5-amino-3-difluoromethyl-3,6-dihydro-2H-[1,4]oxazin-3-yl)-phenyl]-amide;
5-Chloro-3-methyl-pyridine-2-carboxylic acid [3-(5-amino-3-difluoromethyl-3,6-dihydro-2H-[1,4]oxazin-3-yl)-phenyl]-amide;
5-Bromo-pyridine-2-carboxylic acid [3-(5-amino-3-difluoromethyl-3,6-dihydro-2H-[1,4]oxazin-3-yl)-4-fluoro-phenyl]-amide;
5-Chloro-pyridine-2-carboxylic acid [3-(5-amino-3-difluoromethyl-3,6-dihydro-2H-[1,4]oxazin-3-yl)-4-fluoro-phenyl]-amide;
5-Chloro-3-methyl-pyridine-2-carboxylic acid [3-(5-amino-3-difluoromethyl-3,6-dihydro-2H-[1,4]oxazin-3-yl)-4-fluoro-phenyl]-amide;
5-Bromo-3-methyl-pyridine-2-carboxylic acid [3-(5-amino-3-difluoromethyl-3,6-dihydro-2H-[1,4]oxazin-3-yl)-4-fluoro-phenyl]-amide;
3-Chloro-5-trifluoromethyl-pyridine-2-carboxylic acid [3-(5-amino-3-difluoromethyl-3,6-dihydro-2H-[1,4]oxazin-3-yl)-4-fluoro-phenyl]-amide;
5-Trifluoromethyl-pyridine-2-carboxylic acid [3-(5-amino-3-difluoromethyl-3,6-dihydro-2H-[1,4]oxazin-3-yl)-4-fluoro-phenyl]-amide;
5-Methyl-pyrazine-2-carboxylic acid [3-(5-amino-3-difluoromethyl-3,6-dihydro-2H-[1,4]oxazin-3-yl)-4-fluoro-phenyl]-amide;
5-Methoxy-pyridine-2-carboxylic acid [3-(5-amino-3-difluoromethyl-3,6-dihydro-2H-[1,4]oxazin-3-yl)-4-fluoro-phenyl]-amide;
5-Chloro-pyridine-2-carboxylic acid [3-(5-amino-3-trifluoromethyl-3,6-dihydro-2H-[1,4]oxazin-3-yl)-4-fluoro-phenyl]-amide;
5-Bromo-pyrimidine-2-carboxylic acid [3-(5-amino-3-methyl-3,6-dihydro-2H-[1,4]oxazin-3-yl)-5-bromo-phenyl]-amide;
5-Bromo-pyridine-2-carboxylic acid [3-(5-amino-3-methyl-3,6-dihydro-2H-[1,4]oxazin-3-yl)-5-bromo-phenyl]-amide;
5-Cyano-pyridine-2-carboxylic acid [3-(5-amino-3-methyl-3,6-dihydro-2H-[1,4]oxazin-3-yl)-5-bromo-phenyl]-amide;
5-Trifluoromethyl-pyridine-2-carboxylic acid [3-(5-amino-3-methyl-3,6-dihydro-2H-[1,4]oxazin-3-yl)-5-bromo-phenyl]-amide;
5-Bromo-3-methyl-pyridine-2-carboxylic acid [3-(5-amino-3-methyl-3,6-dihydro-2H-[1,4]oxazin-3-yl)-5-bromo-phenyl]-amide;
5-Chloro-pyridine-2-carboxylic acid [3-(5-amino-3-methyl-3,6-dihydro-2H-[1,4]oxazin-3-yl)-5-bromo-phenyl]-amide;
5-Methyl-pyrazine-2-carboxylic acid [3-(5-amino-3-methyl-3,6-dihydro-2H-[1,4]oxazin-3-yl)-5-bromo-phenyl]-amide;
2,5-Dimethyl-oxazole-4-carboxylic acid [3-(5-amino-3-methyl-3,6-dihydro-2H-[1,4]oxazin-3-yl)-5-bromo-phenyl]-amide;
2-Methyl-oxazole-4-carboxylic acid [3-(5-amino-3-methyl-3,6-dihydro-2H-[1,4]oxazin-3-yl)-5-bromo-phenyl]-amide;
5-Bromo-pyridine-2-carboxylic acid [3-(5-amino-3-methyl-3,6-dihydro-2H-[1,4]oxazin-3-yl)-4-fluoro-phenyl]-amide;
5-Methyl-pyrazine-2-carboxylic acid [3-(5-amino-3-methyl-3,6-dihydro-2H-[1,4]oxazin-3-yl)-4-fluoro-phenyl]-amide;
5-Cyano-pyridine-2-carboxylic acid [3-(5-amino-3-methyl-3,6-dihydro-2H-[1,4]oxazin-3-yl)-4-fluoro-phenyl]-amide;
5-Chloro-pyridine-2-carboxylic acid [3-(5-amino-3-methyl-3,6-dihydro-2H-[1,4]oxazin-3-yl)-4-fluoro-phenyl]-amide;
5-Bromo-3-methyl-pyridine-2-carboxylic acid [3-(5-amino-3-methyl-3,6-dihydro-2H-[1,4]oxazin-3-yl)-4-fluoro-phenyl]-amide;
Pyridine-2,5-dicarboxylic acid 5-amide 2-{[3-(5-amino-3-methyl-3,6-dihydro-2H-[1,4]oxazin-3-yl)-4-fluoro-phenyl]-amide};
5-Bromo-pyridine-2-carboxylic acid [3-(5-amino-3,6-dimethyl-6-trifluoromethyl-3,6-dihydro-2H-[1,4]oxazin-3-yl)-phenyl]-amide;
5-Methoxy-pyridine-2-carboxylic acid [3-(5-amino-3,6-dimethyl-6-trifluoromethyl-3,6-di-hydro-2H-[1,4]oxazin-3-yl)-phenyl]-amide;
5-Bromo-pyridine-2-carboxylic acid [3-(5-amino-3,6-di-methyl-6-trifluoromethyl-3,6-di-hydro-2H-[1,4]oxazin-3-yl)-4-fluoro-phenyl]-amide;
5-Methoxy-pyridine-2-carboxylic acid [3-(5-amino-3,6-di-methyl-6-trifluoro [symbol] methyl-3,6-di-hydro-2H-[1,4]oxazin-3-yl)-4-fluoro-phenyl]-amide;
5-Cyano-pyrimidine-2-carboxylic acid [3-(5-amino-3,6-di-methyl-6-trifluoromethyl-3,6-di-hydro-2H-[1,4]oxazin-3-yl)-4-fluoro-phenyl]-amide;
5-Bromo-pyrimidine-2-carboxylic acid [3-(5-amino-3,6-dimethyl-6-trifluoromethyl-3,6-dihydro-2H-[1,4]oxazin-3-yl)-4-fluoro-phenyl]-amide;
5-Cyano-3-methyl-pyridine-2-carboxylic acid [3-((5-amino-3,6-dimethyl-6-trifluoro-methyl-3,6-dihydro-2H-[1,4]oxazin-3-yl)-4-fluoro-phenyl]-amide;
5-Cyano-pyridine-2-carboxylic acid [3-(5-amino-3,6-dimethyl-6-trifluoro-methyl-3,6-dihydro-2H-[1,4]oxazin-3-yl)-4-fluoro-phenyl]-amide;
5-(2-Methoxy-ethoxy)-pyrazine-2-carboxylic acid [3-(5-amino-3,6-dimethyl-6-trifluoromethyl-3,6-dihydro-2H-[1,4]oxazin-3-yl)-4-fluoro-phenyl]-amide;
Cyano-pyridine-2-carboxylic acid [3-(5-amino-3-difluoromethyl-6-methyl-3,6-dihydro-2H-[1,4]oxazin-3-yl)-4-fluoro-phenyl]-amide;
Cyano-pyridine-2-carboxylic acid [3-(5-amino-3-difluoromethyl-6-methyl-3,6-dihydro-2H-[1,4]oxazin-3-yl)-4-fluoro-phenyl]-amide;
5-Bromo-pyridine-2-carboxylic acid[3-(5-amino-3-difluoromethyl-6-methyl-3,6-dihydro-2H-[1,4]oxazin-3-yl)-4-fluoro-phenyl]-amide;
5-Bromo-pyridine-2-carboxylic acid[3-(5-amino-3-difluoromethyl-6-methyl-3,6-dihydro-2H-[1,4]oxazin-3-yl)-4-fluoro-phenyl]-amide;
5-Cyano-pyridine-2-carboxylic acid[3-(5-amino-3-difluoromethyl-6,6-dimethyl-3,6-dihydro-2H-[1,4]oxazin-3-yl)-4-fluoro-phenyl]-amide;
5-Bromo-pyridine-2-carboxylic acid [3-(5-amino-3-difluoromethyl-6,6-dimethyl-3,6-dihydro-2H-[1,4]oxazin-3-yl)-4-fluoro-phenyl]-amide;
5-Bromo-pyridine-2-carboxylic acid [5-(5-amino-3-difluoromethyl-3,6-dihydro-2H-[1,4]oxazin-3-yl)-2,4-difluoro-phenyl]-amide;
5-Cyano-pyridine-2-carboxylic acid [5-(5-amino-3-difluoromethyl-3,6-dihydro-2H-[1,4]oxazin-3-yl)-2,4-difluoro-phenyl]-amide;
5-Bromo-3-methoxy-pyridine-2-carboxylic acid [5-(5-amino-3-difluoromethyl-3,6-dihydro-2H-[1,4]oxazin-3-yl)-2,4-difluoro-phenyl]-amide;
5-Bromo-3-hydroxy-pyridine-2-carboxylic acid [5-(5-amino-3-difluoromethyl-3,6-dihydro-2H-[1,4]oxazin-3-yl)-2,4-difluoro-phenyl]-amide;
5-Chloro-pyridine-2-carboxylic acid [3-(5-amino-3-fluoromethyl-3,6-dihydro-2H-[1,4]oxazin-3-yl)-4-fluoro-phenyl]-amide;
5-Bromo-pyridine-2-carboxylic acid [3-(5-amino-3-fluoromethyl-3,6-dihydro-2H-[1,4]oxazin-3-yl)-4-fluoro-phenyl]-amide;
5-Chloro-pyridine-2-carboxylic acid [3-(5-amino-3-fluoromethyl-3,6-dihydro-2H-[1,4]oxazin-3-yl)-4-fluoro-phenyl]-amide;
3,5-Dichloro-pyridine-2-carboxylic acid [3-(5-amino-3-fluoromethyl-3,6-dihydro-2H-[1,4]oxazin-3-yl)-4-fluoro-phenyl]-amide;
5-Cyano-pyridine-2-carboxylic acid [3-(5-amino-3-fluoromethyl-3,6-dihydro-2H-[1,4]oxazin-3-yl)-4-fluoro-phenyl]-amide;
5-Bromo-3-methyl-pyridine-2-carboxylic acid [3-(5-amino-3-fluoromethyl-3,6-dihydro-2H-[1,4]oxazin-3-yl)-4-fluoro-phenyl]-amide;
5-Bromo-3-methyl-pyridine-2-carboxylic acid [3-(5-amino-3-fluoromethyl-3,6-dihydro-2H-[1,4]oxazin-3-yl)-4-fluoro-phenyl]-amide;
5-Chloro-3-methyl-pyridine-2-carboxylic acid [3-(5-amino-3-fluoromethyl-3,6-dihydro-2H-[1,4]oxazin-3-yl)-4-fluoro-phenyl]-amide;
5-Chloro-4,6-dideutero-3-trideuteromethyl-pyridine-2-carboxylic acid [3-(5-amino-3-fluoromethyl-3,6-dihydro-2H-[1,4]oxazin-3-yl)-4-fluoro-phenyl]-amide;
5-Bromo-3-hydroxy-pyridine-2-carboxylic acid [3-(5-amino-3-fluoromethyl-3,6-dihydro-2H-[1,4]oxazin-3-yl)-4-fluoro-phenyl]-amide;
5-Bromo-3-methoxy-pyridine-2-carboxylic acid [3-(5-amino-3-fluoromethyl-3,6-dihydro-2H-[1,4]oxazin-3-yl)-4-fluoro-phenyl]-amide;
5-Bromo-pyridine-2-carboxylic acid[3-(3-amino-5-difluoromethyl-2,5,6,7-tetrahydro-[1,4]oxazepin-5-yl)-4-fluoro-phenyl]-amide;
5-Bromo-pyridine-2-carboxylic acid[3-(3-amino-5-difluoromethyl-2,5,6,7-tetrahydro-[1,4]oxazepin-5-yl)-4-fluoro-phenyl]-amide;
5-Cyano-pyridine-2-carboxylic acid [3-(3-amino-5-trifluoromethyl-2,5,6,7-tetrahydro-[1,4]oxazepin-5-yl)-4-fluoro-phenyl]-amide;
5-Bromo-pyridine-2-carboxylic acid [3-(3-amino-5-trifluoromethyl-2,5,6,7-tetrahydro-[1,4]oxazepin-5-yl)-4-fluoro-phenyl]-amide;
N-(3-(3-amino-6,6-difluoro-5-methyl-2,5,6,7-tetrahydro-1,4-oxazepin-5-yl)-4-fluorophenyl)-5-chloropicolinamide;
5-Bromo-pyridine-2-carboxylic acid [3-(3-amino-6,6-difluoro-5-methyl-2,5,6,7-tetrahydro-[1,4]oxazepin-5-yl)-4-fluoro-phenyl]-amide;
4-Bromo-furan-2-carboxylic acid [3-(5-amino-3-difluoromethyl-3,6-dihydro-2H-[1,4]oxazin-3-yl)-phenyl]-amide;
6-Hydroxy-pyridazine-3-carboxylic acid [3-(5-amino-3-difluoromethyl-3,6-dihydro-2H-[1,4]oxazin-3-yl)-phenyl]-amide;
2-Methyl-oxazole-4-carboxylic acid [3-(5-amino-3-difluoromethyl-3,6-dihydro-2H-[1,4]oxazin-3-yl)-phenyl]-amide;
2-Ethyl-oxazole-4-carboxylic acid [3-(5-amino-3-difluoromethyl-3,6-dihydro-2H-[1,4]oxazin-3-yl)-phenyl]-amide;
2,5-Dimethyl-oxazole-4-carboxylic acid [3-(5-amino-3-difluoromethyl-3,6-dihydro-2H-[1,4]oxazin-3-yl)-phenyl]-amide;
5-Bromo-3-methoxy-pyridine-2-carboxylic acid [3-(5-amino-3-difluoromethyl-3,6-dihydro-2H-[1,4]oxazin-3-yl)-phenyl]-amide;
5-Bromo-3-hydroxy-pyridine-2-carboxylic acid [3-(5-amino-3-difluoromethyl-3,6-dihydro-2H-[1,4]oxazin-3-yl)-phenyl]-amide;
5-(2-Methoxy-ethoxy)-pyridine-2-carboxylic acid [3-(5-amino-3-difluoromethyl-3,6-dihydro-2H-[1,4]oxazin-3-yl)-phenyl]-amide;
5-Difluoromethyl-pyrazine-2-carboxylic acid [3-(5-amino-3-difluoromethyl-3,6-dihydro-2H-[1,4]oxazin-3-yl)-phenyl]-amide;
3,5-Dichloro-pyridine-2-carboxylic acid [3-(5-amino-3-difluoromethyl-3,6-dihydro-2H-[1,4]oxazin-3-yl)-4-fluoro-phenyl]-amide;
5-Cyano-pyridine-2-carboxylic acid [3-(5-amino-3-difluoromethyl-3,6-dihydro-2H-[1,4]oxazin-3-yl)-4-fluoro-phenyl]-amide;
3,5-Difluoro-pyridine-2-carboxylic acid [3-(5-amino-3-difluoromethyl-3,6-dihydro-2H-[1,4]oxazin-3-yl)-4-fluoro-phenyl]-amide;
5-Bromo-3-methyl-benzofuran-2-carboxylic acid [3-(5-amino-3-difluoromethyl-3,6-dihydro-2H-[1,4]oxazin-3-yl)-4-fluoro-phenyl]-amide;
5-Chloro-4,6-dideutero-3-trideuteromethyl-pyridine-2-carboxylic acid [3-(5-amino-3-difluoromethyl-3,6-dihydro-2H-[1,4]oxazin-3-yl)-4-fluoro-phenyl]-amide;
5-Bromo-4,6-dideutero-3-trideuteromethyl-pyridine-2-carboxylic acid [3-(5-amino-3-difluoromethyl-3,6-dihydro-2H-[1,4]oxazin-3-yl)-4-fluoro-phenyl]-amide;
5-Bromo-3-chloro-pyridine-2-carboxylic acid [3-(5-amino-3-difluoromethyl-3,6-dihydro-2H-[1,4]oxazin-3-yl)-4-fluoro-phenyl]-amide;
5-Bromo-pyridine-2-carboxylic acid [3-(5-amino-3-difluoromethyl-3,6-dihydro-2H-[1,4]oxazin-3-yl)-4-fluoro-phenyl]-amide;
5-Bromo-3-hydroxy-pyridine-2-carboxylic acid [3-(5-amino-3-difluoromethyl-3,6-dihydro-2H-[1,4]oxazin-3-yl)-4-fluoro-phenyl]-amide;
5-Ethoxy-pyridine-2-carboxylic acid [3-(5-amino-3-difluoromethyl-3,6-dihydro-2H-[1,4]oxazin-3-yl)-4-fluoro-phenyl]-amide;
5-Chloro-pyrimidine-2-carboxylic acid [3-(5-amino-3-difluoromethyl-3,6-dihydro-2H-[1,4]oxazin-3-yl)-4-fluoro-phenyl]-amide;
5-Difluoromethyl-pyrazine-2-carboxylic acid [3-(5-amino-3-difluoromethyl-3,6-dihydro-2H-[1,4]oxazin-3-yl)-4-fluoro-phenyl]-amide;
5-(2,2-Difluoro-ethoxy)-pyrazine-2-carboxylic acid [3-(5-amino-3-difluoromethyl-3,6-dihydro-2H-[1,4]oxazin-3-yl)-4-fluoro-phenyl]-amide;
5-(2,2,2-Trifluoro-ethoxy)-pyrazine-2-carboxylic acid [3-(5-amino-3-difluoromethyl-3,6-dihydro-2H-[1,4]oxazin-3-yl)-4-fluoro-phenyl]-amide;
4-Bromo-furan-2-carboxylic acid [3-(5-amino-3-difluoromethyl-3,6-dihydro-2H-[1,4]oxazin-3-yl)-4-fluoro-phenyl]-amide;
Pyrazolo[1,5-a]pyridine-2-carboxylic acid [3-(5-amino-3 difluoromethyl-3,6-dihydro-2H [1,4]oxazin-3-yl)-4-fluoro-phenyl]-amide;
2-Methyl-oxazole-4-carboxylic acid [3-(5-amino-3-difluoromethyl-3,6-dihydro-2H-[1,4]oxazin-3-yl)-4-fluoro-phenyl]-amide;
2,5-Dimethyl-oxazole-4-carboxylic acid [3-(5-amino-3-difluoromethyl-3,6-dihydro-2H-[1,4]oxazin-3-yl)-4-fluoro-phenyl]-amide;
Imidazo[1,2-a]pyridine-2-carboxylic acid [3-(5-amino-3-difluoromethyl-3,6-dihydro-2H-[1,4]oxazin-3-yl)-4-fluoro-phenyl]-amide;
5-Bromo-3-methoxy-pyridine-2-carboxylic acid [3-(5-amino-3-difluoromethyl-3,6-dihydro-2H-[1,4]oxazin-3-yl)-4-fluoro-phenyl]-amide;
2-Ethyl-oxazole-4-carboxylic acid [3-(5-amino-3-difluoromethyl-3,6-dihydro-2H-[1,4]oxazin-3-yl)-4-fluoro-phenyl]-amide;
5-Difluoromethyl-pyridine-2-carboxylic acid [3-(5-amino-3-difluoromethyl-3,6-dihydro-2H-[1,4]oxazin-3-yl)-4-fluoro-phenyl]-amide;
1-Methyl-1H-imidazole-2 carboxylic acid [3-(5-amino-3 difluoromethyl-3,6-dihydro-2H [1,4]oxazin-3-yl)-4-fluoro-phenyl]-amide;
6-Hydroxy-pyridazine-3-carboxylic acid [3-(5-amino-3-difluoromethyl-3,6-dihydro-2H-[1,4]oxazin-3-yl)-4-fluoro-phenyl]-amide;
5-(2-Methoxy-ethoxy)-pyrazine-2-carboxylic acid [3-(5-amino-3-difluoromethyl-3,6-dihydro-2H-[1,4]oxazin-3-yl)-4-fluoro-phenyl]-amide;
5-(2-Fluoro-ethoxy)-pyrazine-2-carboxylic acid [3-(5-amino-3-difluoromethyl-3,6-dihydro-2H-[1,4]oxazin-3-yl)-4-fluoro-phenyl]-amide;
5-Chloro-pyridine-2-carboxylic acid [3-(5-amino-3-difluoromethyl-3,6-dihydro-2H-[1,4]oxazin-3-yl)-4-fluoro-phenyl]-amide;
5-Difluoromethoxy-pyridine-2-carboxylic acid [3-(5-amino-3-difluoromethyl-3,6-dihydro-2H-[1,4]oxazin-3-yl)-4-fluoro-phenyl]-amide;
5-Fluoromethoxy-pyridine-2-carboxylic acid [3-(5-amino-3-difluoromethyl-3,6-dihydro-2H-[1,4]oxazin-3-yl)-4-fluoro-phenyl]-amide;
5-Chloro-3-fluoro-pyridine-2-carboxylic acid [3-(5-amino-3-difluoromethyl-3,6-dihydro-2H-[1,4]oxazin-3-yl)-4-fluoro-phenyl]-amide;
5-Chloro-pyrazine-2-carboxylic acid [3-(5-amino-3-difluoromethyl-3,6-dihydro-2H-[1,4]oxazin-3-yl)-4-fluoro-phenyl]-amide;
5-Methyl-1H-pyrazole-3-carboxylic acid [3-(5-amino-3-difluoromethyl-3,6-dihydro-2H-[1,4]oxazin-3-yl)-4-fluoro-phenyl]-amide;
5-Methyl-pyridine-2-carboxylic acid [3-(5-amino-3-difluoromethyl-3,6-dihydro-2H-[1,4]oxazin-3-yl)-4-fluoro-phenyl]-amide;
5-Cyano-3-methyl-pyridine-2-carboxylic acid [3-(5-amino-3-difluoromethyl-3,6-dihydro-2H-[1,4]oxazin-3-yl)-4-fluoro-phenyl]-amide;
5-Hydroxy-pyrazine-2-carboxylic acid [3-(5-amino-3-difluoromethyl-3,6-dihydro-2H-[1,4]oxazin-3-yl)-4-fluoro-phenyl]-amide;
5-Methoxy-pyrazine-2-carboxylic acid [3-(5-amino-3-difluoromethyl-3,6-dihydro-2H-[1,4]oxazin-3-yl)-4-fluoro-phenyl]-amide;
5-Cyano-4,6-dideutero-3-trideuteromethyl-pyridine-2-carboxylic acid [3-(5-amino-3-difluoromethyl-3,6-dihydro-2H-[1,4]oxazin-3-yl)-4-fluoro-phenyl]-amide;
2-Methyl-thiazole-4-carboxylic acid [3-(5-amino-3-difluoromethyl-3,6-dihydro-2H-[1,4]oxazin-3-yl)-4-fluoro-phenyl]-amide;
5-Methyl-thiazole-2-carboxylic acid [3-(5-amino-3-difluoromethyl-3,6-dihydro-2H-[1,4]oxazin-3-yl)-4-fluoro-phenyl]-amide;
1-Methyl-1H-pyrazole-3-carboxylic acid [3-(5-amino-3-difluoromethyl-3,6-dihydro-2H-[1,4]oxazin-3-yl)-4-fluoro-phenyl]-amide;
1-Methyl-4-nitro-1H-pyrazole-3-carboxylic acid [3-(5-amino-3-difluoromethyl-3,6-dihydro-2H-[1,4]oxazin-3-yl)-4-fluoro-phenyl]-amide;
3-Amino-5-chloro-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Chloro-5-trifluoromethyl-pyridine-2-carboxylic acid [3-(3-amino-6,6-difluoro-5-methyl-2,5,6,7-tetrahydro-[1,4]oxazepin-5-yl)-4-fluoro-phenyl]-amide;
5-Bromo-3-methoxy-pyridine-2-carboxylic acid [3-(3-amino-6,6 difluoro-5-methyl-2,5,6,7 tetrahydro-[1,4]oxazepin-5-yl)-4 fluoro-phenyl]-amide;
5-Cyano-pyridine-2-carboxylic acid [3-(3-amino-6,6-difluoro-5 methyl-2,5,6,7-tetrahydro[1,4]oxazepin-5-yl)-4 fluoro-phenyl]-amide;
7H-Pyrrolo[2,3-d]pyrimidine-6-carboxylic acid [3-(5-amino-3-difluoromethyl-3,6-dihydro-2H-[1,4]oxazin-3-yl)-4-fluoro-phenyl]-amide;
3-Amino-5-(2-methoxy-ethoxy)-pyrazine-2-carboxylic acid [3-(5-amino-3-difluoromethyl-3,6-dihydro-2H-[1,4]oxazin-3-yl)-4-fluoro-phenyl]-amide;
(5-Difluoromethyl-5-{5-[(5-ethyl-pyridine-2-carbonyl)-amino]-2-fluoro-phenyl}-5,6-dihydro-2H-[1,4]oxazin-3-yl)-carbamic acid tert-butyl ester;
3-Amino-5-chloro-pyridine-2-carboxylic acid [3-(5-amino-3-difluoromethyl-3,6-dihydro-2H-[1,4]oxazin-3-yl)-4-fluoro-phenyl]-amide;
3-Chloro-5-methoxy-pyridine-2-carboxylic acid [3-(5-amino-3-difluoromethyl-3,6-dihydro-2H-[1,4]oxazin-3-yl)-4-fluoro-phenyl]-amide;
6-Oxo-1,6-dihydro-pyridine-3-carboxylic acid [3-(5-amino-3-difluoromethyl-3,6-dihydro-2H-[1,4]oxazin-3-yl)-4-fluoro-phenyl]-amide;
Pyrrolo[1,2-c]pyrimidine-3-carboxylic acid [3-(5-amino-3-difluoromethyl-3,6-dihydro-2H-[1,4]oxazin-3-yl)-4-fluoro-phenyl]-amide;
5-But-2-ynyloxy-pyrazine-2-carboxylic acid [3-(5-amino-3-difluoromethyl-3,6-dihydro-2H-[1,4]oxazin-3-yl)-4-fluoro-phenyl]-amide;
3-Amino-5-bromo-pyridine-2-carboxylic acid [3-(5-amino-3-difluoromethyl-3,6-dihydro-2H-[1,4]oxazin-3-yl)-4-fluoro-phenyl]-amide;
1-Ethyl-1H-imidazole-2-carboxylic acid [3-(5-amino-3-difluoromethyl-3,6-dihydro-2H-[1,4]oxazin-3-yl)-4-fluoro-phenyl]-amide;
5-Prop-2-ynyloxy-pyrazine-2-carboxylic acid [3-(5-amino-3-difluoromethyl-3,6-dihydro-2H-[1,4]oxazin-3-yl)-4-fluoro-phenyl]-amide;
5-Amino-2-methyl-oxazole-4-carboxylic acid [3-(5-amino-3-difluoromethyl-3,6-dihydro-2H-[1,4]oxazin-3-yl)-4-fluoro-phenyl]-amide;
5-Chloro-3-hydroxy-pyridine-2-carboxylic acid [3-(5-amino-3-difluoromethyl-3,6-dihydro-2H-[1,4]oxazin-3-yl)-4-fluoro-phenyl]-amide;
5-Isopropoxy-pyrazine-2-carboxylic acid [3-(5-amino-3-difluoromethyl-3,6-dihydro-2H-[1,4]oxazin-3-yl)-4-fluoro-phenyl]-amide;
5-Ethoxy-pyrazine-2-carboxylic acid [3-(5-amino-3-difluoromethyl-3,6-dihydro-2H-[1,4]oxazin-3-yl)-4-fluoro-phenyl]-amide;
5-Dimethylaminomethyl-3-methyl-benzofuran-2-carboxylic acid [3-(5-amino-3-difluoromethyl-3,6-dihydro-2H-[1,4]oxazin-3-yl)-4-fluoro-phenyl]-amide;
1,5-Dimethyl-1H-[1,2,3]triazole-4-carboxylic acid [3-(5-amino-3-difluoromethyl-3,6-dihydro-2H-[1,4]oxazin-3-yl)-4-fluoro-phenyl]-amide;
5-Methoxy-3-methyl-pyrazine-2-carboxylic acid [3-(5-amino-3-difluoromethyl-3,6-dihydro-2H-[1,4]oxazin-3-yl)-4-fluoro-phenyl]-amide;
3-Amino-5-methoxy-pyrazine-2-carboxylic acid [3-(5-amino-3-difluoromethyl-3,6-dihydro-2H-[1,4]oxazin-3-yl)-4-fluoro-phenyl]-amide;
5-Difluoromethoxy-3-methyl-pyridine-2-carboxylic acid [3-(5-amino-3-difluoromethyl-3,6-dihydro-2H-[1,4]oxazin-3-yl)-4-fluoro-phenyl]-amide;
5-Fluoromethoxy-3-methyl-pyridine-2-carboxylic acid [3-(5-amino-3-difluoromethyl-3,6-dihydro-2H-[1,4]oxazin-3-yl)-4-fluoro-phenyl]-amide;
5-(2-Methoxy-ethoxy)-3-methyl-pyrazine-2-carboxylic acid [3-(5-amino-3-difluoromethyl-3,6-dihydro-2H-[1,4]oxazin-3-yl)-4-fluoro-phenyl]-amide;
3,5-Dimethoxy-pyridine-2-carboxylic acid [3-(5-amino-3-difluoromethyl-3,6-dihydro-2H-[1,4]oxazin-3-yl)-4-fluoro-phenyl]-amide;
5-(2-Methoxy-ethoxy)-pyridine-2-carboxylic acid [3-(5-amino-3-difluoromethyl-3,6-dihydro-2H-[1,4]oxazin-3-yl)-4-fluoro-phenyl]-amide;
3-Fluoro-5-(2-methoxy-ethoxy)-pyridine-2-carboxylic acid [3-(5-amino-3-difluoromethyl-3,6-dihydro-2H-[1,4]oxazin-3-yl)-4-fluoro-phenyl]-amide;
5-But-2-ynyloxy-3-methyl-pyrazine-2-carboxylic acid [3-(5-amino-3-difluoromethyl-3,6-dihydro-2H-[1,4]oxazin-3-yl)-4-fluoro-phenyl]-amide;
3-Chloro-5-methoxymethyl-pyridine-2-carboxylic acid [3-(5-amino-3-difluoromethyl-3,6-dihydro-2H-[1,4]oxazin-3-yl)-4-fluoro-phenyl]-amide;
3-Chloro-5-fluoromethoxy-pyridine-2-carboxylic acid [3-(5-amino-3-difluoromethyl-3,6-dihydro-2H-[1,4]oxazin-3-yl)-4-fluoro-phenyl]-amide; and
3-Chloro-5-difluoromethoxy-pyridine-2-carboxylic acid [3-(5-amino-3-difluoromethyl-3,6-dihydro-2H-[1,4]oxazin-3-yl)-4-fluoro-phenyl]-amide.

In another embodiment, the invention relates to a method of treating diseases associated with inhibition of BACE-2 activity comprising administering to a subject a therapeutically effective amount of a compound of the invention, or a pharmaceutically acceptable salt thereof, which is selected from:
Furan-2-carboxylic acid [3-(5-amino-3-methyl-3,6-dihydro-2H-[1,4]oxazin-3-yl)-phenyl]-amide;
5-Bromo-pyridine-2-carboxylic acid [3-(5-amino-3-methyl-3,6-dihydro-2H-[1,4]oxazin-3-yl)-phenyl]-amide;
5-Bromo-pyridine-2-carboxylic acid [3-((R)-5-amino-3-methyl-3,6-di [symbol] hydro-2H-[1,4]oxazin-3-yl)-phenyl]-amide;
5-Bromo-pyridine-2-carboxylic acid [3-((S)-5-amino-3-methyl-3,6-di [symbol] hydro-2H-[1,4]oxazin-3-yl)-phenyl]-amide;
2-Methyl-oxazole-4-carboxylic acid [3-(5-amino-3-methyl-3,6-dihydro-2H-[1,4]oxazin-3-yl)-phenyl]-amide;
5-Methyl-pyrazine-2-carboxylic acid [3-(5-amino-3-methyl-3,6-dihydro-2H-[1,4]oxazin-3-yl)-phenyl]-amide;
5-Bromo-pyrimidine-2-carboxylic acid [3-(5-amino-3-methyl-3,6-dihydro-2H-[1,4]oxazin-3-yl)-phenyl]-amide;
Imidazo[1,2-a]pyridine-2-carboxylic acid [3-(5-amino-3-methyl-3,6-dihydro-2H-[1,4]oxazin-3-yl)-phenyl]-amide;
3-Fluoro-pyridine-2-carboxylic acid [3-(5-amino-3-methyl-3,6-dihydro-2H-[1,4]oxazin-3-yl)-phenyl]-amide;
2,5-Dimethyl-oxazole-4-carboxylic acid [3-(5-amino-3-methyl-3,6-dihydro-2H-[1,4]oxazin-3-yl)-phenyl]-amide;
2-Methyl-thiazole-4-carboxylic acid [3-(5-amino-3-methyl-3,6-dihydro-2H-[1,4]oxazin-3-yl)-phenyl]-amide;
6-Hydroxy-pyridazine-3-carboxylic acid [3-(5-amino-3-methyl-3,6-dihydro-2H-[1,4]oxazin-3-yl)-phenyl]-amide;
Pyridine-2-carboxylic acid [3-(5-amino-3-methyl-3,6-dihydro-2H-[1,4]oxazin-3-yl)-phenyl]-amide;
5-Cyano-pyridine-2-carboxylic acid [3-(5-amino-3-methyl-3,6-dihydro-2H-[1,4]oxazin-3-yl)-phenyl]-amide;
5-(3-Trifluoromethyl-pyrazol-1-yl)-pyrazine-2-carboxylic acid [3-(5-amino-3-methyl-3,6-dihydro-2H-[1,4]oxazin-3-yl)-phenyl]-amide;
5-(3-Methyl-pyrazol-1-yl)-pyrazine-2-carboxylic acid [3-(5-amino-3-methyl-3,6-dihydro-2H-[1,4]oxazin-3-yl)-phenyl]-amide;
3-Chloro-5-trifluoromethyl-pyridine-2-carboxylic acid [3-(5-amino-3-methyl-3,6-dihydro-2H-[1,4]oxazin-3-yl)-phenyl]-amide;
5-Methoxy-pyrazine-2-carboxylic acid [3-(5-amino-3-methyl-3,6-dihydro-2H-[1,4]oxazin-3-yl)-phenyl]-amide;
5-Hydroxy-pyrazine-2-carboxylic acid [3-(5-amino-3-methyl-3,6-dihydro-2H-[1,4]oxazin-3-yl)-phenyl]-amide;
4-Bromo-furan-2-carboxylic acid [3-(5-amino-3-methyl-3,6-dihydro-2H-[1,4]oxazin-3-yl)-phenyl]-amide;
5-Chloro-pyridine-2-carboxylic acid [3-(5-amino-3-methyl-3,6-dihydro-2H-[1,4]oxazin-3-yl)-phenyl]-amide;
5-Trifluoromethyl-pyridine-2-carboxylic acid [3-(5-amino-3-methyl-3,6-dihydro-2H-[1,4]oxazin-3-yl)-phenyl]-amide;
5-Trifluoromethyl-furan-3-carboxylic acid [3-(5-amino-3-methyl-3,6-dihydro-2H-[1,4]oxazin-3-yl)-phenyl]-amide;
N-[3-(5-Amino-3-methyl-3,6-dihydro-2H-[1,4]oxazin-3-yl)-phenyl]-4-bromo-benzamide;
5-Methyl-pyridine-2-carboxylic acid [3-(5-amino-3-methyl-3,6-dihydro-2H-[1,4]oxazin-3-yl)-phenyl]-amide;
N-[3-(5-Amino-3-methyl-3,6-dihydro-2H-[1,4]oxazin-3-yl)-phenyl]-nicotinamide;
5-Bromo-3-methyl-pyridine-2-carboxylic acid [3-((R)-5-amino-3-methyl-3,6-dihydro-2H-[1,4]oxazin-3-yl)-phenyl]-amide;
5-Methoxy-pyridine-2-carboxylic acid [3-((R)-5-amino-3-methyl-3,6-dihydro-2H-[1,4]oxazin-3-yl)-phenyl]-amide;
5-Bromo-pyridine-2-carboxylic acid [3-(5-amino-3-fluoromethyl-3,6-dihydro-2H-[1,4]oxazin-3-yl)-phenyl]-amide;
5-Bromo-pyridine-2-carboxylic acid [3-(5-amino-3-fluoromethyl-3,6-dihydro-2H-[1,4]oxazin-3-yl)-5-chloro-phenyl]-amide;
5-Bromo-pyridine-2-carboxylic acid [3-((R)-5-amino-3-fluoromethyl-3,6-dihydro-2H-[1,4]oxazin-3-yl)-phenyl]-amide;
5-Bromo-pyridine-2-carboxylic acid [3-(5-amino-3-difluoromethyl-3,6-dihydro-2H-[1,4]oxazin-3-yl)-phenyl]-amide;
5-Chloro-pyridine-2-carboxylic acid [3-(5-amino-3-difluoromethyl-3,6-dihydro-2H-[1,4]oxazin-3-yl)-phenyl]-amide;
5-Cyano-pyridine-2-carboxylic acid [3-(5-amino-3-difluoromethyl-3,6-dihydro-2H-[1,4]oxazin-3-yl)-phenyl]-amide;
5-Bromo-3-methyl-pyridine-2-carboxylic acid [3-(5-amino-3-difluoromethyl-3,6-dihydro-2H-[1,4]oxazin-3-yl)-phenyl]-amide;
5-Methyl-pyrazine-2-carboxylic acid [3-(5-amino-3-difluoromethyl-3,6-dihydro-2H-[1,4]oxazin-3-yl)-phenyl]-amide;
5-Trifluoromethyl-pyridine-2-carboxylic acid [3-(5-amino-3-difluoromethyl-3,6-dihydro-2H-[1,4]oxazin-3-yl)-phenyl]-amide;
3-Chloro-5-trifluoromethyl-pyridine-2-carboxylic acid [3-(5-amino-3-difluoromethyl-3,6-dihydro-2H-[1,4]oxazin-3-yl)-phenyl]-amide;
5-Chloro-3-methyl-pyridine-2-carboxylic acid [3-(5-amino-3-difluoromethyl-3,6-dihydro-2H-[1,4]oxazin-3-yl)-phenyl]-amide;
5-Bromo-pyridine-2-carboxylic acid [3-(5-amino-3-difluoromethyl-3,6-dihydro-2H-[1,4]oxazin-3-yl)-4-fluoro-phenyl]-amide;
5-Chloro-pyridine-2-carboxylic acid [3-(5-amino-3-difluoromethyl-3,6-dihydro-2H-[1,4]oxazin-3-yl)-4-fluoro-phenyl]-amide;
5-Chloro-3-methyl-pyridine-2-carboxylic acid [3-((R)-5-amino-3-difluoromethyl-3,6-dihydro-2H-[1,4]oxazin-3-yl)-4-fluoro-phenyl]-amide;
5-Bromo-3-methyl-pyridine-2-carboxylic acid [3-((R)-5-amino-3-difluoromethyl-3,6-dihydro-2H-[1,4]oxazin-3-yl)-4-fluoro-phenyl]-amide;
3-Chloro-5-trifluoromethyl-pyridine-2-carboxylic acid [3-((R)-5-amino-3-difluoromethyl-3,6-dihydro-2H-[1,4]oxazin-3-yl)-4-fluoro-phenyl]-amide;
5-Trifluoromethyl-pyridine-2-carboxylic acid [3-((R)-5-amino-3-difluoromethyl-3,6-dihydro-2H-[1,4]oxazin-3-yl)-4-fluoro-phenyl]-amide;
5-Methyl-pyrazine-2-carboxylic acid [3-((R)-5-amino-3-difluoromethyl-3,6-dihydro-2H-[1,4]oxazin-3-yl)-4-fluoro-phenyl]-amide;
5-Methoxy-pyridine-2-carboxylic acid [3-((R)-5-amino-3-difluoromethyl-3,6-dihydro-2H-[1,4]oxazin-3-yl)-4-fluoro-phenyl]-amide;
5-Chloro-pyridine-2-carboxylic acid [3-(5-amino-3-trifluoromethyl-3,6-dihydro-2H-[1,4]oxazin-3-yl)-4-fluoro-phenyl]-amide;
5-Bromo-pyrimidine-2-carboxylic acid [3-((R)-5-amino-3-methyl-3,6-dihydro-2H-[1,4]oxazin-3-yl)-5-bromo-phenyl]-amide;
5-Bromo-pyridine-2-carboxylic acid [3-((R)-5-amino-3-methyl-3,6-dihydro-2H-[1,4]oxazin-3-yl)-5-bromo-phenyl]-amide;
5-Cyano-pyridine-2-carboxylic acid [3-((R)-5-amino-3-methyl-3,6-dihydro-2H-[1,4]oxazin-3-yl)-5-bromo-phenyl]-amide;
5-Trifluoromethyl-pyridine-2-carboxylic acid [3-((R)-5-amino-3-methyl-3,6-dihydro-2H-[1,4]oxazin-3-yl)-5-bromo-phenyl]-amide;
5-Bromo-3-methyl-pyridine-2-carboxylic acid [3-((R)-5-amino-3-methyl-3,6-dihydro-2H-[1,4]oxazin-3-yl)-5-bromo-phenyl]-amide;
5-Chloro-pyridine-2-carboxylic acid [3-((R)-5-amino-3-methyl-3,6-dihydro-2H-[1,4]oxazin-3-yl)-5-bromo-phenyl]-amide;
5-Methyl-pyrazine-2-carboxylic acid [3-((R)-5-amino-3-methyl-3,6-dihydro-2H-[1,4]oxazin-3-yl)-5-bromo-phenyl]-amide;
2,5-Dimethyl-oxazole-4-carboxylic acid [3-((R)-5-amino-3-methyl-3,6-dihydro-2H-[1,4]oxazin-3-yl)-5-bromo-phenyl]-amide;
2-Methyl-oxazole-4-carboxylic acid [3-((R)-5-amino-3-methyl-3,6-dihydro-2H-[1,4]oxazin-3-yl)-5-bromo-phenyl]-amide;
5-Bromo-pyridine-2-carboxylic acid [3-(5-amino-3-methyl-3,6-dihydro-2H-[1,4]oxazin-3-yl)-4-fluoro-phenyl]-amide;
5-Methyl-pyrazine-2-carboxylic acid [3-(5-amino-3-methyl-3,6-dihydro-2H-[1,4]oxazin-3-yl)-4-fluoro-phenyl]-amide;
5-Cyano-pyridine-2-carboxylic acid [3-(5-amino-3-methyl-3,6-dihydro-2H-[1,4]oxazin-3-yl)-4-fluoro-phenyl]-amide;
5-Chloro-pyridine-2-carboxylic acid [3-(5-amino-3-methyl-3,6-dihydro-2H-[1,4]oxazin-3-yl)-4-fluoro-phenyl]-amide;
5-Bromo-3-methyl-pyridine-2-carboxylic acid [3-(5-amino-3-methyl-3,6-dihydro-2H-[1,4]oxazin-3-yl)-4-fluoro-phenyl]-amide;
Pyridine-2,5-dicarboxylic acid 5-amide 2-{[3-(5-amino-3-methyl-3,6-dihydro-2H-[1,4]oxazin-3-yl)-4-fluoro-phenyl]-amide};
5-Bromo-pyridine-2-carboxylic acid [3-((3R,6R)-5-amino-3,6-dimethyl-6-trifluoromethyl-3,6-dihydro-2H-[1,4]oxazin-3-yl)-phenyl]-amide;
5-Methoxy-pyridine-2-carboxylic acid [3-((3R,6R)-5-amino-3,6-dimethyl-6-trifluoromethyl-3,6-dihydro-2H-[1,4]oxazin-3-yl)-phenyl]-amide;
5-Bromo-pyridine-2-carboxylic acid [3-((3R,6R)-5-amino-3,6-di-methyl-6-trifluoromethyl-3,6-di-hydro-2H-[1,4]oxazin-3-yl)-4-fluoro-phenyl]-amide;
5-Methoxy-pyridine-2-carboxylic acid [3-((3R,6R)-5-amino-3,6-di-methyl-6-trifluoro [symbol] methyl-3,6-di-hydro-2H-[1,4]oxazin-3-yl)-4-fluoro-phenyl]-amide;
5-Cyano-pyrimidine-2-carboxylic acid [3-((3R,6R)-5-amino-3,6-di-methyl-6-trifluoromethyl-3,6-di-hydro-2H-[1,4]oxazin-3-yl)-4-fluoro-phenyl]-amide;
5-Bromo-pyrimidine-2-carboxylic acid [3-((3R,6R)-5-amino-3,6-dimethyl-6-trifluoro-methyl-3,6-dihydro-2H-[1,4]oxazin-3-yl)-4-fluoro-phenyl]-amide;
5-Cyano-3-methyl-pyridine-2-carboxylic acid [3-((3R,6R)-5-amino-3,6-dimethyl-6-trifluoro-methyl-3,6-dihydro-2H-[1,4]oxazin-3-yl)-4-fluoro-phenyl]-amide;
5-Cyano-pyridine-2-carboxylic acid [3-((3R,6R)-5-amino-3,6-dimethyl-6-trifluoro-methyl-3,6-dihydro-2H-[1,4]oxazin-3-yl)-4-fluoro-phenyl]-amide;
5-(2-Methoxy-ethoxy)-pyrazine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
Cyano-pyridine-2-carboxylic acid [3-((3R,6R)-5-amino-3-difluoromethyl-6-methyl-3,6-dihydro-2H-[1,4]oxazin-3-yl)-4-fluoro-phenyl]-amide;
Cyano-pyridine-2-carboxylic acid [3-((3R,6S)-5-amino-3-difluoromethyl-6-methyl-3,6-dihydro-2H-[1,4]oxazin-3-yl)-4-fluoro-phenyl]-amide;
5-Bromo-pyridine-2-carboxylic acid[3-((3R,6R)-5-amino-3-difluoromethyl-6-methyl-3,6-dihydro-2H-[1,4]oxazin-3-yl)-4-fluoro-phenyl]-amide;
5-Bromo-pyridine-2-carboxylic acid[3-((3S,6R)-5-amino-3-difluoromethyl-6-methyl-3,6-dihydro-2H-[1,4]oxazin-3-yl)-4-fluoro-phenyl]-amide;
5-Cyano-pyridine-2-carboxylic acid[3-(5-amino-3-difluoromethyl-6,6-dimethyl-3,6-dihydro-2H-[1,4]oxazin-3-yl)-4-fluoro-phenyl]-amide;
5-Bromo-pyridine-2-carboxylic acid [3-(5-amino-3-difluoromethyl-6,6-dimethyl-3,6-dihydro-2H-[1,4]oxazin-3-yl)-4-fluoro-phenyl]-amide;
5-Bromo-pyridine-2-carboxylic acid [5-(5-amino-3-difluoromethyl-3,6-dihydro-2H-[1,4]oxazin-3-yl)-2,4-difluoro-phenyl]-amide;
5-Cyano-pyridine-2-carboxylic acid [5-(5-amino-3-difluoromethyl-3,6-dihydro-2H-[1,4]oxazin-3-yl)-2,4-difluoro-phenyl]-amide;
5-Bromo-3-methoxy-pyridine-2-carboxylic acid [5-(5-amino-3-difluoromethyl-3,6-dihydro-2H-[1,4]oxazin-3-yl)-2,4-difluoro-phenyl]-amide;
5-Bromo-3-hydroxy-pyridine-2-carboxylic acid [5-(5-amino-3-difluoromethyl-3,6-dihydro-2H-[1,4]oxazin-3-yl)-2,4-difluoro-phenyl]-amide;
5-Chloro-pyridine-2-carboxylic acid [3-((R)-5-amino-3-fluoromethyl-3,6-dihydro-2H-[1,4]oxazin-3-yl)-4-fluoro-phenyl]-amide;
5-Bromo-pyridine-2-carboxylic acid [3-(5-amino-3-fluoromethyl-3,6-dihydro-2H-[1,4]oxazin-3-yl)-4-fluoro-phenyl]-amide;
5-Chloro-pyridine-2-carboxylic acid [3-(5-amino-3-fluoromethyl-3,6-dihydro-2H-[1,4]oxazin-3-yl)-4-fluoro-phenyl]-amide;
3,5-Dichloro-pyridine-2-carboxylic acid [3-(5-amino-3-fluoromethyl-3,6-dihydro-2H-[1,4]oxazin-3-yl)-4-fluoro-phenyl]-amide;
5-Cyano-pyridine-2-carboxylic acid [3-(5-amino-3-fluoromethyl-3,6-dihydro-2H-[1,4]oxazin-3-yl)-4-fluoro-phenyl]-amide;
5-Bromo-3-methyl-pyridine-2-carboxylic acid [3-(5-amino-3-fluoromethyl-3,6-dihydro-2H-[1,4]oxazin-3-yl)-4-fluoro-phenyl]-amide;
5-Bromo-3-methyl-pyridine-2-carboxylic acid [3-((R)-5-amino-3-fluoromethyl-3,6-dihydro-2H-[1,4]oxazin-3-yl)-4-fluoro-phenyl]-amide;
5-Chloro-3-methyl-pyridine-2-carboxylic acid [3-((R)-5-amino-3-fluoromethyl-3,6-dihydro-2H-[1,4]oxazin-3-yl)-4-fluoro-phenyl]-amide;
5-Chloro-4,6-dideutero-3-trideuteromethyl-pyridine-2-carboxylic acid [3-((R)-5-amino-3-fluoromethyl-3,6-dihydro-2H-[1,4]oxazin-3-yl)-4-fluoro-phenyl]-amide;
5-Bromo-3-hydroxy-pyridine-2-carboxylic acid [3-((R)-5-amino-3-fluoromethyl-3,6-dihydro-2H-[1,4]oxazin-3-yl)-4-fluoro-phenyl]-amide;
5-Bromo-3-methoxy-pyridine-2-carboxylic acid [3-((R)-5-amino-3-fluoromethyl-3,6-dihydro-2H-[1,4]oxazin-3-yl)-4-fluoro-phenyl]-amide;
5-Bromo-pyridine-2-carboxylic acid[3-((S)-3-amino-5-difluoromethyl-2,5,6,7-tetrahydro-[1,4]oxazepin-5-yl)-4-fluoro-phenyl]-amide;
5-Bromo-pyridine-2-carboxylic acid[3-((S)-3-amino-5-difluoromethyl-2,5,6,7-tetrahydro-[1,4]oxazepin-5-yl)-4-fluoro-phenyl]-amide;
5-Cyano-pyridine-2-carboxylic acid [3-(3-amino-5-trifluoromethyl-2,5,6,7-tetrahydro-[1,4]oxazepin-5-yl)-4-fluoro-phenyl]-amide;
5-Bromo-pyridine-2-carboxylic acid [3-(3-amino-5-trifluoromethyl-2,5,6,7-tetrahydro-[1,4]oxazepin-5-yl)-4-fluoro-phenyl]-amide;
N-(3-(3-amino-6,6-difluoro-5-methyl-2,5,6,7-tetrahydro-1,4-oxazepin-5-yl)-4-fluorophenyl)-5-chloropicolinamide;
5-Bromo-pyridine-2-carboxylic acid [3-(3-amino-6,6-difluoro-5-methyl-2,5,6,7-tetrahydro-[1,4]oxazepin-5-yl)-4-fluoro-phenyl]-amide;
4-Bromo-furan-2-carboxylic acid [3-(5-amino-3-difluoromethyl-3,6-dihydro-2H-[1,4]oxazin-3-yl)-phenyl]-amide;
6-Hydroxy-pyridazine-3-carboxylic acid [3-(5-amino-3-difluoromethyl-3,6-dihydro-2H-[1,4]oxazin-3-yl)-phenyl]-amide;
2-Methyl-oxazole-4-carboxylic acid [3-(5-amino-3-difluoromethyl-3,6-dihydro-2H-[1,4]oxazin-3-yl)-phenyl]-amide;
2-Ethyl-oxazole-4-carboxylic acid [3-(5-amino-3-difluoromethyl-3,6-dihydro-2H-[1,4]oxazin-3-yl)-phenyl]-amide;
2,5-Dimethyl-oxazole-4-carboxylic acid [3-(5-amino-3-difluoromethyl-3,6-dihydro-2H-[1,4]oxazin-3-yl)-phenyl]-amide;
5-Bromo-3-methoxy-pyridine-2-carboxylic acid [3-(5-amino-3-difluoromethyl-3,6-dihydro-2H-[1,4]oxazin-3-yl)-phenyl]-amide;
5-Bromo-3-hydroxy-pyridine-2-carboxylic acid [3-(5-amino-3-difluoromethyl-3,6-dihydro-2H-[1,4]oxazin-3-yl)-phenyl]-amide;
5-(2-Methoxy-ethoxy)-pyridine-2-carboxylic acid [3-(5-amino-3-difluoromethyl-3,6-dihydro-2H-[1,4]oxazin-3-yl)-phenyl]-amide;
5-Difluoromethyl-pyrazine-2-carboxylic acid [3-(5-amino-3-difluoromethyl-3,6-dihydro-2H-[1,4]oxazin-3-yl)-phenyl]-amide;
3,5-Dichloro-pyridine-2-carboxylic acid [3-((R)-5-amino-3-difluoromethyl-3,6-dihydro-2H-[1,4]oxazin-3-yl)-4-fluoro-phenyl]-amide;
5-Cyano-pyridine-2-carboxylic acid [3-((R)-5-amino-3-difluoromethyl-3,6-dihydro-2H-[1,4]oxazin-3-yl)-4-fluoro-phenyl]-amide;
3,5-Difluoro-pyridine-2-carboxylic acid [3-((R)-5-amino-3-difluoromethyl-3,6-dihydro-2H-[1,4]oxazin-3-yl)-4-fluoro-phenyl]-amide;
5-Bromo-3-methyl-benzofuran-2-carboxylic acid [3-((R)-5-amino-3-difluoromethyl-3,6-dihydro-2H-[1,4]oxazin-3-yl)-4-fluoro-phenyl]-amide;
5-Chloro-4,6-dideutero-3-trideuteromethyl-pyridine-2-carboxylic acid [3-((R)-5-amino-3-difluoromethyl-3,6-dihydro-2H-[1,4]oxazin-3-yl)-4-fluoro-phenyl]-amide;
5-Bromo-4,6-dideutero-3-trideuteromethyl-pyridine-2-carboxylic acid [3-((R)-5-amino-3-difluoromethyl-3,6-dihydro-2H-[1,4]oxazin-3-yl)-4-fluoro-phenyl]-amide;
5-Bromo-3-chloro-pyridine-2-carboxylic acid [3-((R)-5-amino-3-difluoromethyl-3,6-dihydro-2H-[1,4]oxazin-3-yl)-4-fluoro-phenyl]-amide;
5-Bromo-pyridine-2-carboxylic acid [3-((R)-5-amino-3-difluoromethyl-3,6-dihydro-2H-[1,4]oxazin-3-yl)-4-fluoro-phenyl]-amide;
5-Bromo-3-hydroxy-pyridine-2-carboxylic acid [3-((R)-5-amino-3-difluoromethyl-3,6-dihydro-2H-[1,4]oxazin-3-yl)-4-fluoro-phenyl]-amide;
5-Ethoxy-pyridine-2-carboxylic acid [3-((R)-5-amino-3-difluoromethyl-3,6-dihydro-2H-[1,4]oxazin-3-yl)-4-fluoro-phenyl]-amide;
5-Chloro-pyrimidine-2-carboxylic acid [3-((R)-5-amino-3-difluoromethyl-3,6-dihydro-2H-[1,4]oxazin-3-yl)-4-fluoro-phenyl]-amide;
5-Difluoromethyl-pyrazine-2-carboxylic acid [3-((R)-5-amino-3-difluoromethyl-3,6-dihydro-2H-[1,4]oxazin-3-yl)-4-fluoro-phenyl]-amide;
5-(2,2-Difluoro-ethoxy)-pyrazine-2-carboxylic acid [3-((R)-5-amino-3-difluoromethyl-3,6-dihydro-2H-[1,4]oxazin-3-yl)-4-fluoro-phenyl]-amide;
5-(2,2,2-Trifluoro-ethoxy)-pyrazine-2-carboxylic acid [3-((R)-5-amino-3-difluoromethyl-3,6-dihydro-2H-[1,4]oxazin-3-yl)-4-fluoro-phenyl]-amide;
4-Bromo-furan-2-carboxylic acid [3-((R)-5-amino-3-difluoromethyl-3,6-dihydro-2H-[1,4]oxazin-3-yl)-4-fluoro-phenyl]-amide;
Pyrazolo[1,5-a]pyridine-2-carboxylic acid [3-((R)-5-amino-3 difluoromethyl-3,6-dihydro-2H [1,4]oxazin-3-yl)-4-fluoro-phenyl]-amide;
2-Methyl-oxazole-4-carboxylic acid [3-((R)-5-amino-3-difluoromethyl-3,6-dihydro-2H-[1,4]oxazin-3-yl)-4-fluoro-phenyl]-amide;
2,5-Dimethyl-oxazole-4-carboxylic acid [3-((R)-5-amino-3-difluoromethyl-3,6-dihydro-2H-[1,4]oxazin-3-yl)-4-fluoro-phenyl]-amide;
Imidazo[1,2-a]pyridine-2-carboxylic acid [3-((R)-5-amino-3-difluoromethyl-3,6-dihydro-2H-[1,4]oxazin-3-yl)-4-fluoro-phenyl]-amide;
5-Bromo-3-methoxy-pyridine-2-carboxylic acid [3-((R)-5-amino-3-difluoromethyl-3,6-dihydro-2H-[1,4]oxazin-3-yl)-4-fluoro-phenyl]-amide;
2-Ethyl-oxazole-4-carboxylic acid [3-((R)-5-amino-3-difluoromethyl-3,6-dihydro-2H-[1,4]oxazin-3-yl)-4-fluoro-phenyl]-amide;
5-Difluoromethyl-pyridine-2-carboxylic acid [3-((R)-5-amino-3-difluoromethyl-3,6-dihydro-2H-[1,4]oxazin-3-yl)-4-fluoro-phenyl]-amide;
1-Methyl-1H-imidazole-2 carboxylic acid [3-((R)-5-amino-3 difluoromethyl-3,6-dihydro-2H [1,4]oxazin-3-yl)-4-fluoro-phenyl]-amide;
6-Hydroxy-pyridazine-3-carboxylic acid [3-((R)-5-amino-3-difluoromethyl-3,6-dihydro-2H-[1,4]oxazin-3-yl)-4-fluoro-phenyl]-amide;
5-(2-Methoxy-ethoxy)-pyrazine-2-carboxylic acid [3-((R)-5-amino-3-difluoromethyl-3,6-dihydro-2H-[1,4]oxazin-3-yl)-4-fluoro-phenyl]-amide;
5-(2-Fluoro-ethoxy)-pyrazine-2-carboxylic acid [3-((R)-5-amino-3-difluoromethyl-3,6-dihydro-2H-[1,4]oxazin-3-yl)-4-fluoro-phenyl]-amide;
5-Chloro-pyridine-2-carboxylic acid [3-((R)-5-amino-3-difluoromethyl-3,6-dihydro-2H-[1,4]oxazin-3-yl)-4-fluoro-phenyl]-amide;
5-Difluoromethoxy-pyridine-2-carboxylic acid [3-((R)-5-amino-3-difluoromethyl-3,6-dihydro-2H-[1,4]oxazin-3-yl)-4-fluoro-phenyl]-amide;
5-Fluoromethoxy-pyridine-2-carboxylic acid [3-((R)-5-amino-3-difluoromethyl-3,6-dihydro-2H-[1,4]oxazin-3-yl)-4-fluoro-phenyl]-amide;
5-Chloro-3-fluoro-pyridine-2-carboxylic acid [3-((R)-5-amino-3-difluoromethyl-3,6-dihydro-2H-[1,4]oxazin-3-yl)-4-fluoro-phenyl]-amide;
5-Chloro-pyrazine-2-carboxylic acid [3-((R)-5-amino-3-difluoromethyl-3,6-dihydro-2H-[1,4]oxazin-3-yl)-4-fluoro-phenyl]-amide;
5-Methyl-1H-pyrazole-3-carboxylic acid [3-((R)-5-amino-3-difluoromethyl-3,6-dihydro-2H-[1,4]oxazin-3-yl)-4-fluoro-phenyl]-amide;
5-Methyl-pyridine-2-carboxylic acid [3-((R)-5-amino-3-difluoromethyl-3,6-dihydro-2H-[1,4]oxazin-3-yl)-4-fluoro-phenyl]-amide;
5-Cyano-3-methyl-pyridine-2-carboxylic acid [3-((R)-5-amino-3-difluoromethyl-3,6-dihydro-2H-[1,4]oxazin-3-yl)-4-fluoro-phenyl]-amide;
5-Hydroxy-pyrazine-2-carboxylic acid [3-((R)-5-amino-3-difluoromethyl-3,6-dihydro-2H-[1,4]oxazin-3-yl)-4-fluoro-phenyl]-amide;
5-Methoxy-pyrazine-2-carboxylic acid [3-((R)-5-amino-3-difluoromethyl-3,6-dihydro-2H-[1,4]oxazin-3-yl)-4-fluoro-phenyl]-amide;
5-Cyano-4,6-dideutero-3-trideuteromethyl-pyridine-2-carboxylic acid [3-((R)-5-amino-3-difluoromethyl-3,6-dihydro-2H-[1,4]oxazin-3-yl)-4-fluoro-phenyl]-amide;
2-Methyl-thiazole-4-carboxylic acid [3-((R)-5-amino-3-difluoromethyl-3,6-dihydro-2H-[1,4]oxazin-3-yl)-4-fluoro-phenyl]-amide;
5-Methyl-thiazole-2-carboxylic acid [3-((R)-5-amino-3-difluoromethyl-3,6-dihydro-2H-[1,4]oxazin-3-yl)-4-fluoro-phenyl]-amide;
1-Methyl-1H-pyrazole-3-carboxylic acid [3-((R)-5-amino-3-difluoromethyl-3,6-dihydro-2H-[1,4]oxazin-3-yl)-4-fluoro-phenyl]-amide;
1-Methyl-4-nitro-1H-pyrazole-3-carboxylic acid [3-((R)-5-amino-3-difluoromethyl-3,6-dihydro-2H-[1,4]oxazin-3-yl)-4-fluoro-phenyl]-amide;
3-Amino-5-chloro-pyridine-2-carboxylic acid [3-((3R,6R)-5-amino-3,6-dimethyl-6-trifluoro-methyl-3,6-dihydro-2H-[1,4]oxazin-3-yl)-4-fluoro-phenyl]-amide;
3-Chloro-5-trifluoromethyl-pyridine-2-carboxylic acid [3-(3-amino-6,6-difluoro-5-methyl-2,5,6,7-tetrahydro-[1,4]oxazepin-5-yl)-4-fluoro-phenyl]-amide;
5-Bromo-3-methoxy-pyridine-2-carboxylic acid [3-(3-amino-6,6 difluoro-5-methyl-2,5,6,7 tetrahydro-[1,4]oxazepin-5-yl)-4 fluoro-phenyl]-amide;
5-Cyano-pyridine-2-carboxylic acid [3-(3-amino-6,6-difluoro-5 methyl-2,5,6,7-tetrahydro[1,4]oxazepin-5-yl)-4 fluoro-phenyl]-amide;
7H-Pyrrolo[2,3-d]pyrimidine-6-carboxylic acid [3-((R)-5-amino-3-difluoromethyl-3,6-dihydro-2H-[1,4]oxazin-3-yl)-4-fluoro-phenyl]-amide;
3-Amino-5-(2-methoxy-ethoxy)-pyrazine-2-carboxylic acid [3-((R)-5-amino-3-difluoromethyl-3,6-dihydro-2H-[1,4]oxazin-3-yl)-4-fluoro-phenyl]-amide;
((R)-5-Difluoromethyl-5-{5-[(5-ethyl-pyridine-2-carbonyl)-amino]-2-fluoro-phenyl}-5,6-dihydro-2H-[1,4]oxazin-3-yl)-carbamic acid tert-butyl ester;
3-Amino-5-chloro-pyridine-2-carboxylic acid [3-((R)-5-amino-3-difluoromethyl-3,6-dihydro-2H-[1,4]oxazin-3-yl)-4-fluoro-phenyl]-amide;
3-Chloro-5-methoxy-pyridine-2-carboxylic acid [3-((R)-5-amino-3-difluoromethyl-3,6-dihydro-2H-[1,4]oxazin-3-yl)-4-fluoro-phenyl]-amide;
6-Oxo-1,6-dihydro-pyridine-3-carboxylic acid [3-((R)-5-amino-3-difluoromethyl-3,6-dihydro-2H-[1,4]oxazin-3-yl)-4-fluoro-phenyl]-amide;
Pyrrolo[1,2-c]pyrimidine-3-carboxylic acid [3-((R)-5-amino-3-difluoromethyl-3,6-dihydro-2H-[1,4]oxazin-3-yl)-4-fluoro-phenyl]-amide;
5-But-2-ynyloxy-pyrazine-2-carboxylic acid [3-((R)-5-amino-3-difluoromethyl-3,6-dihydro-2H-[1,4]oxazin-3-yl)-4-fluoro-phenyl]-amide;
3-Amino-5-bromo-pyridine-2-carboxylic acid [3-((R)-5-amino-3-difluoromethyl-3,6-dihydro-2H-[1,4]oxazin-3-yl)-4-fluoro-phenyl]-amide;
1-Ethyl-1H-imidazole-2-carboxylic acid [3-((R)-5-amino-3-difluoromethyl-3,6-dihydro-2H-[1,4]oxazin-3-yl)-4-fluoro-phenyl]-amide;
5-Prop-2-ynyloxy-pyrazine-2-carboxylic acid [3-((R)-5-amino-3-difluoromethyl-3,6-dihydro-2H-[1,4]oxazin-3-yl)-4-fluoro-phenyl]-amide;
5-Amino-2-methyl-oxazole-4-carboxylic acid [3-((R)-5-amino-3-difluoromethyl-3,6-dihydro-2H-[1,4]oxazin-3-yl)-4-fluoro-phenyl]-amide;
5-Chloro-3-hydroxy-pyridine-2-carboxylic acid [3-((R)-5-amino-3-difluoromethyl-3,6-dihydro-2H-[1,4]oxazin-3-yl)-4-fluoro-phenyl]-amide;
5-Isopropoxy-pyrazine-2-carboxylic acid [3-((R)-5-amino-3-difluoromethyl-3,6-dihydro-2H-[1,4]oxazin-3-yl)-4-fluoro-phenyl]-amide;
5-Ethoxy-pyrazine-2-carboxylic acid [3-((R)-5-amino-3-difluoromethyl-3,6-dihydro-2H-[1,4]oxazin-3-yl)-4-fluoro-phenyl]-amide;
5-Dimethylaminomethyl-3-methyl-benzofuran-2-carboxylic acid [3-((R)-5-amino-3-difluoromethyl-3,6-dihydro-2H-[1,4]oxazin-3-yl)-4-fluoro-phenyl]-amide;
1,5-Dimethyl-1H-[1,2,3]triazole-4-carboxylic acid [3-((R)-5-amino-3-difluoromethyl-3,6-dihydro-2H-[1,4]oxazin-3-yl)-4-fluoro-phenyl]-amide;
5-Methoxy-3-methyl-pyrazine-2-carboxylic acid [3-((R)-5-amino-3-difluoromethyl-3,6-dihydro-2H-[1,4]oxazin-3-yl)-4-fluoro-phenyl]-amide;
3-Amino-5-methoxy-pyrazine-2-carboxylic acid [3-((R)-5-amino-3-difluoromethyl-3,6-dihydro-2H-[1,4]oxazin-3-yl)-4-fluoro-phenyl]-amide;
5-Difluoromethoxy-3-methyl-pyridine-2-carboxylic acid [3-((R)-5-amino-3-difluoromethyl-3,6-dihydro-2H-[1,4]oxazin-3-yl)-4-fluoro-phenyl]-amide;
5-Fluoromethoxy-3-methyl-pyridine-2-carboxylic acid [3-((R)-5-amino-3-difluoromethyl-3,6-dihydro-2H-[1,4]oxazin-3-yl)-4-fluoro-phenyl]-amide;
5-(2-Methoxy-ethoxy)-3-methyl-pyrazine-2-carboxylic acid [3-((R)-5-amino-3-difluoromethyl-3,6-dihydro-2H-[1,4]oxazin-3-yl)-4-fluoro-phenyl]-amide;
3,5-Dimethoxy-pyridine-2-carboxylic acid [3-((R)-5-amino-3-difluoromethyl-3,6-dihydro-2H-[1,4]oxazin-3-yl)-4-fluoro-phenyl]-amide;
5-(2-Methoxy-ethoxy)-pyridine-2-carboxylic acid [3-((R)-5-amino-3-difluoromethyl-3,6-dihydro-2H-[1,4]oxazin-3-yl)-4-fluoro-phenyl]-amide;
3-Fluoro-5-(2-methoxy-ethoxy)-pyridine-2-carboxylic acid [3-((R)-5-amino-3-difluoromethyl-3,6-dihydro-2H-[1,4]oxazin-3-yl)-4-fluoro-phenyl]-amide;
5-But-2-ynyloxy-3-methyl-pyrazine-2-carboxylic acid [3-((R)-5-amino-3-difluoromethyl-3,6-dihydro-2H-[1,4]oxazin-3-yl)-4-fluoro-phenyl]-amide;
3-Chloro-5-methoxymethyl-pyridine-2-carboxylic acid [3-((R)-5-amino-3-difluoromethyl-3,6-dihydro-2H-[1,4]oxazin-3-yl)-4-fluoro-phenyl]-amide;
3-Chloro-5-fluoromethoxy-pyridine-2-carboxylic acid [3-((R)-5-amino-3-difluoromethyl-3,6-dihydro-2H-[1,4]oxazin-3-yl)-4-fluoro-phenyl]-amide; and
3-Chloro-5-difluoromethoxy-pyridine-2-carboxylic acid [3-((R)-5-amino-3-difluoromethyl-3,6-dihydro-2H-[1,4]oxazin-3-yl)-4-fluoro-phenyl]-amide.

In another embodiment, the invention relates to a method of treating diseases associated with inhibition of BACE-2 activity comprising administering to a subject a therapeutically effective amount of a compound of the invention, or a pharmaceutically acceptable salt thereof, which is selected from:
5-Chloro-3-methoxymethyl-pyridine-2-carboxylic acid [3-(5-amino-3-difluoromethyl-3,6-dihydro-2H-[1,4]oxazin-3-yl)-4-fluoro-phenyl]-amide;
3-Chloro-5-cyano-pyridine-2-carboxylic acid [3-(5-amino-3-difluoromethyl-3,6-dihydro-2H-[1,4]oxazin-3-yl)-4-fluoro-phenyl]-amide;
3-Amino-5-tris-deutero-methoxy-pyrazine-2-carboxylic acid [3-(5-amino-3-difluoromethyl-3,6-dihydro-2H-[1,4]oxazin-3-yl)-4-fluoro-phenyl]-amide;
3-Amino-5-prop-2-ynyloxy-pyrazine-2-carboxylic acid [3-(5-amino-3-difluoromethyl-3,6-dihydro-2H-[1,4]oxazin-3-yl)-4-fluoro-phenyl]-amide;
3-Chloro-1H-pyrrolo[2,3-b]pyridine-6-carboxylic acid [3-(5-amino-3-difluoromethyl-3,6-dihydro-2H-[1,4]oxazin-3-yl)-4-fluoro-phenyl]-amide;
3-Amino-5-difluoromethyl-pyrazine-2-carboxylic acid [3-(5-amino-3-difluoromethyl-3,6-dihydro-2H-[1,4]oxazin-3-yl)-4-fluoro-phenyl]-amide;
5-Methoxy-3-methyl-pyridine-2-carboxylic acid [3-(5-amino-3-difluoromethyl-3,6-dihydro-2H-[1,4]oxazin-3-yl)-4-fluoro-phenyl]-amide;
5-Difluoromethyl-3-methyl-pyridine-2-carboxylic acid [3-(5-amino-3-difluoromethyl-3,6-dihydro-2H-[1,4]oxazin-3-yl)-4-fluoro-phenyl]-amide;
5-Fluoro-3-trideuteromethoxymethyl-pyridine-2-carboxylic acid [3-(5-amino-3-difluoromethyl-3,6-dihydro-2H-[1,4]oxazin-3-yl)-4-fluoro-phenyl]-amide;
5-Trideuteromethoxy-3-trideuteromethoxymethyl-pyridine-2-carboxylic acid [3-(5-amino-3-difluoromethyl-3,6-dihydro-2H-[1,4]oxazin-3-yl)-4-fluoro-phenyl]-amide;
3-Amino-5-cyano-pyridine-2-carboxylic acid [3-(5-amino-3-difluoromethyl-3,6-dihydro-2H-[1,4]oxazin-3-yl)-4-fluoro-phenyl]-amide;
3-Amino-5-difluoromethyl-pyridine-2-carboxylic acid [3-(5-amino-3-difluoromethyl-3,6-dihydro-2H-[1,4]oxazin-3-yl)-4-fluoro-phenyl]-amide;
5-Trideuteromethoxy-3-methyl-pyridine-2-carboxylic acid [3-(5-amino-3-difluoromethyl-3,6-dihydro-2H-[1,4]oxazin-3-yl)-4-fluoro-phenyl]-amide;
5-Cyano-3-methyl-pyridine-2-carboxylic acid [3-(5-amino-3-difluoromethyl-3,6-dihydro-2H-[1,4]oxazin-3-yl)-4,5-difluoro-phenyl]-amide;
3-Chloro-5-trideuteromethoxy-pyridine-2-carboxylic acid [3-(5-amino-3-difluoromethyl-3,6-dihydro-2H-[1,4]oxazin-3-yl)-4,5-difluoro-phenyl]-amide;
4,6-Dideutero-5-chloro-3-trideuteromethyl-pyridine-2-carboxylic acid [3-(5-amino-3-difluoromethyl-3,6-dihydro-2H-[1,4]oxazin-3-yl)-4,5-difluoro-phenyl]-amide;
3-Chloro-5-cyano-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Chloro-5-methoxy-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Chloro-5-difluoromethoxy-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
5-Chloro-3-methyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
5-Chloro-3-fluoro-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Chloro-5-trideutero-methoxy-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
2,5-Dimethyl-oxazole-4-carboxylic acid [3-(5-amino-3,6-dimethyl-6-trifluoromethyl-3,6-dihydro-2H-[1,4]oxazin-3-yl)-4-fluoro-phenyl]-amide;
5-Difluoromethoxy-3-methyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Amino-5-prop-2-ynyloxy-pyrazine-2-carboxylic acid [3-(5-amino-3,6-dimethyl-6-trifluoromethyl-3,6-dihydro-2H-[1,4]oxazin-3-yl)-4-fluoro-phenyl]-amide;
3-Amino-5-cyano-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
5-Difluoromethyl-3-methyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Amino-5-difluoromethyl-pyrazine-2-carboxylic acid [3-(5-amino-3,6-dimethyl-6-trifluoromethyl-3,6-dihydro-2H-[1,4]oxazin-3-yl)-4-fluoro-phenyl]-amide;
3-Amino-5-difluoromethyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
4-Difluoromethyl-6-methoxy-pyridazine-3-carboxylic acid [3-(5-amino-3,6-dimethyl-6-trifluoromethyl-3,6-dihydro-2H-[1,4]oxazin-3-yl)-4-fluoro-phenyl]-amide;
5-Cyano-3-trideuteromethoxymethyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Chloro-5-difluormethyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Chloro-5-trideuteromethoxy-dideuteromethyl-1H-pyrrolo[2,3-b]pyridine-6-carboxylic acid [3-(5-amino-3,6-dimethyl-6-trifluoromethyl-3,6-dihydro-2H-[1,4]oxazin-3-yl)-4-fluoro-phenyl]-amide;
3-Chloro-5-trifluoromethyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
5-Fluoro-3-methyl-pyridine-2-carboxylic acid [3-(5-amino-3,6-dimethyl-6-trifluoro-methyl-3,6-dihydro-2H-[1,4]oxazin-3-yl)-4-fluoro-phenyl]-amide;
5-Trideuteromethoxy-3-methyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
5-Cyano-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
5-Difluoromethoxy-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
5-Chloro-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
5-Cyano-3-methyl-pyridine-2-carboxylic acid [3-(5-amino-3,6,6-tris-fluoromethyl-3,6-dihydro-2H-[1,4]oxazin-3-yl)-4-fluoro-phenyl]-amide;
5-Difluoromethyl-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
3-Amino-5-difluoromethyl-pyrazine-2-carboxylic acid [3-(5-amino-6,6-bis-fluoromethyl-3-methyl-3,6-dihydro-2H-[1,4]oxazin-3-yl)-4-fluoro-phenyl]-amide;
3-Amino-5-difluoromethyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
3-Amino-5-cyano-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
5-Difluoromethyl-3-methyl-pyridine-2-carboxylic acid [3-(5-amino-3,6,6-tris-fluoromethyl-3,6-dihydro-2H-[1,4]oxazin-3-yl)-4-fluoro-phenyl]-amide;
3-Chloro-5-difluoromethyl-pyridine-2-carboxylic acid [3-(5-amino-3,6,6-tris-fluoromethyl-3,6-dihydro-2H-[1,4]oxazin-3-yl)-4-fluoro-phenyl]-amide;
3-Chloro-5-trifluoromethyl-pyridine-2-carboxylic acid [3-(5-amino-3,6,6-tris-fluoromethyl-3,6-dihydro-2H-[1,4]oxazin-3-yl)-4-fluoro-phenyl]-amide;
3,5-Dichloro-pyridine-2-carboxylic acid [3-(5-amino-3,6,6-tris-fluoromethyl-3,6-dihydro-2H-[1,4]oxazin-3-yl)-4-fluoro-phenyl]-amide;
3-Amino-5-cyano-pyridine-2-carboxylic acid [3-(5-amino-3,6,6-tris-fluoromethyl-3,6-dihydro-2H-[1,4]oxazin-3-yl)-4-fluoro-phenyl]-amide;
3-Chloro-5-cyano-pyridine-2-carboxylic acid [3-(5-amino-3,6,6-tris-fluoromethyl-3,6-dihydro-2H-[1,4]oxazin-3-yl)-4-fluoro-phenyl]-amide;
N-[3-(5-Amino-3,6-dimethyl-6-trifluoromethyl-3,6-dihydro-2H-[1,4]oxazin-3-yl)-4-fluoro-phenyl]-6-methoxy-2-methyl-nicotinamide;
N-[3-(5-Amino-3,6-dimethyl-6-trifluoromethyl-3,6-dihydro-2H-[1,4]oxazin-3-yl)-4-fluoro-phenyl]-6-trideuteromethoxy-2-methyl-nicotinamide;
2-Amino-N-[3-(5-amino-3,6-dimethyl-6-trifluoromethyl-3,6-dihydro-2H-[1,4]oxazin-3-yl)-4-fluoro-phenyl]-6-ethoxy-nicotinamide;
2-Amino-N-[3-(5-amino-3,6-dimethyl-6-trifluoromethyl-3,6-dihydro-2H-[1,4]oxazin-3-yl)-4-fluoro-phenyl]-6-methoxy-nicotinamide;
2-Amino-N-[3-(5-amino-3,6-dimethyl-6-trifluoromethyl-3,6-dihydro-2H-[1,4]oxazin-3-yl)-4-fluoro-phenyl]-6-trideuteromethoxy-nicotinamide;
2-Amino-N-[3-(5-amino-3,6-dimethyl-6-trifluoromethyl-3,6-dihydro-2H-[1,4]oxazin-3-yl)-4-fluoro-phenyl]-6-pentadeuteroethoxy-nicotinamide;
N-[3-(5-Amino-3,6-dimethyl-6-trifluoromethyl-3,6-dihydro-2H-[1,4]oxazin-3-yl)-4-fluoro-phenyl]-2-chloro-6-methoxy-nicotinamide;
N-[3-(5-Amino-3,6-dimethyl-6-trifluoromethyl-3,6-dihydro-2H-[1,4]oxazin-3-yl)-4-fluoro-phenyl]-2-chloro-6-ethoxy-nicotinamide;
2-Amino-N-[3-(5-amino-3,6-dimethyl-6-trifluoromethyl-3,6-dihydro-2H-[1,4]oxazin-3-yl)-4-fluoro-phenyl]-6-cyclopropylmethoxy-nicotinamide; and
2-Amino-N-[3-(5-amino-3,6-dimethyl-6-trifluoromethyl-3,6-dihydro-2H-[1,4]oxazin-3-yl)-4-fluoro-phenyl]-6-(2,2,2-trifluoro-ethoxy)-nicotinamide.

In another embodiment, the invention relates to a method of treating diseases associated with inhibition of BACE-2 activity comprising administering to a subject a therapeutically effective amount of a compound of the invention, or a pharmaceutically acceptable salt thereof, which is selected from:
5-Chloro-3-methoxymethyl-pyridine-2-carboxylic acid [3-((R)-5-amino-3-difluoromethyl-3,6-dihydro-2H-[1,4]oxazin-3-yl)-4-fluoro-phenyl]-amide;
3-Chloro-5-cyano-pyridine-2-carboxylic acid [3-((R)-5-amino-3-difluoromethyl-3,6-dihydro-2H-[1,4]oxazin-3-yl)-4-fluoro-phenyl]-amide;
3-Amino-5-tris-deutero-methoxy-pyrazine-2-carboxylic acid [3-((R)-5-amino-3-difluoromethyl-3,6-dihydro-2H-[1,4]oxazin-3-yl)-4-fluoro-phenyl]-amide;
3-Amino-5-prop-2-ynyloxy-pyrazine-2-carboxylic acid [3-((R)-5-amino-3-difluoromethyl-3,6-dihydro-2H-[1,4]oxazin-3-yl)-4-fluoro-phenyl]-amide;
3-Chloro-1H-pyrrolo[2,3-b]pyridine-6-carboxylic acid [3-((R)-5-amino-3-difluoromethyl-3,6-dihydro-2H-[1,4]oxazin-3-yl)-4-fluoro-phenyl]-amide;
3-Amino-5-difluoromethyl-pyrazine-2-carboxylic acid [3-((R)-5-amino-3-difluoromethyl-3,6-dihydro-2H-[1,4]oxazin-3-yl)-4-fluoro-phenyl]-amide;
5-Methoxy-3-methyl-pyridine-2-carboxylic acid [3-((R)-5-amino-3-difluoromethyl-3,6-dihydro-2H-[1,4]oxazin-3-yl)-4-fluoro-phenyl]-amide;
5-Difluoromethyl-3-methyl-pyridine-2-carboxylic acid [3-((R)-5-amino-3-difluoromethyl-3,6-dihydro-2H-[1,4]oxazin-3-yl)-4-fluoro-phenyl]-amide;
5-Fluoro-3-trideuteromethoxymethyl-pyridine-2-carboxylic acid [3-((R)-5-amino-3-difluoromethyl-3,6-dihydro-2H-[1,4]oxazin-3-yl)-4-fluoro-phenyl]-amide;
5-Trideuteromethoxy-3-trideuteromethoxymethyl-pyridine-2-carboxylic acid [3-((R)-5-amino-3-difluoromethyl-3,6-dihydro-2H-[1,4]oxazin-3-yl)-4-fluoro-phenyl]-amide;
3-Amino-5-cyano-pyridine-2-carboxylic acid [3-((R)-5-amino-3-difluoromethyl-3,6-dihydro-2H-[1,4]oxazin-3-yl)-4-fluoro-phenyl]-amide;
3-Amino-5-difluoromethyl-pyridine-2-carboxylic acid [3-((R)-5-amino-3-difluoromethyl-3,6-dihydro-2H-[1,4]oxazin-3-yl)-4-fluoro-phenyl]-amide;
5-Trideuteromethoxy-3-methyl-pyridine-2-carboxylic acid [3-((R)-5-amino-3-difluoromethyl-3,6-dihydro-2H-[1,4]oxazin-3-yl)-4-fluoro-phenyl]-amide;
5-Cyano-3-methyl-pyridine-2-carboxylic acid [3-((R)-5-amino-3-difluoromethyl-3,6-dihydro-2H-[1,4]oxazin-3-yl)-4,5-difluoro-phenyl]-amide;
3-Chloro-5-trideuteromethoxy-pyridine-2-carboxylic acid [3-((R)-5-amino-3-difluoromethyl-3,6-dihydro-2H-[1,4]oxazin-3-yl)-4,5-difluoro-phenyl]-amide;
4,6-Dideutero-5-chloro-3-trideuteromethyl-pyridine-2-carboxylic acid [3-((R)-5-amino-3-difluoromethyl-3,6-dihydro-2H-[1,4]oxazin-3-yl)-4,5-difluoro-phenyl]-amide;
3-Chloro-5-cyano-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Chloro-5-methoxy-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Chloro-5-difluoromethoxy-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Chloro-3-methyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Chloro-3-fluoro-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Chloro-5-trideutero-methoxy-pyridine-2-carboxylic acid [3-((3R,6R)-5-amino-3,6-dimethyl-6-trifluoromethyl-3,6-di hydro-2H-[1,4]oxazin-3-yl)-4-fluoro-phenyl]-amide;
2,5-Dimethyl-oxazole-4-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Difluoromethoxy-3-methyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Amino-5-prop-2-ynyloxy-pyrazine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Amino-5-cyano-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Difluoromethyl-3-methyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Amino-5-difluoromethyl-pyrazine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Amino-5-difluoromethyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
4-Difluoromethyl-6-methoxy-pyridazine-3-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Cyano-3-trideuteromethoxymethyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Chloro-5-difluormethyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Chloro-5-trideuteromethoxy-dideuteromethyl-1H-pyrrolo[2,3-b]pyridine-6-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Chloro-5-trifluoromethyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Fluoro-3-methyl-pyridine-2-carboxylic acid [3-((3R,6R)-5-amino-3,6-dimethyl-6-trifluoro-methyl-3,6-dihydro-2H-[1,4]oxazin-3-yl)-4-fluoro-phenyl]-amide;
5-Trideuteromethoxy-3-methyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Cyano-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
5-Cyano-3-methyl-pyridine-2-carboxylic acid [3-((R)-5-amino-6,6-bis-fluoromethyl-3-methyl-3,6-dihydro-2H-[1,4]oxazin-3-yl)-4-fluoro-phenyl]-amide;
5-Difluoromethoxy-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
5-Difluoromethoxy-3-methyl-pyridine-2-carboxylic acid [3-((R)-5-amino-6,6-bis-fluoromethyl-3-methyl-3,6-dihydro-2H-[1,4]oxazin-3-yl)-4-fluoro-phenyl]-amide;
5-Chloro-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
5-Cyano-3-methyl-pyridine-2-carboxylic acid [3-((R)-5-amino-3,6,6-tris-fluoromethyl-3,6-dihydro-2H-[1,4]oxazin-3-yl)-4-fluoro-phenyl]-amide;
5-Difluoromethyl-3-methyl-pyridine-2-carboxylic acid [3-((R)-5-amino-6,6-bis-fluoromethyl-3-methyl-3,6-dihydro-2H-[1,4]oxazin-3-yl)-4-fluoro-phenyl]-amide;
3-Amino-5-difluoromethyl-pyrazine-2-carboxylic acid [3-((R)-5-amino-6,6-bis-fluoromethyl-3-methyl-3,6-dihydro-2H-[1,4]oxazin-3-yl)-4-fluoro-phenyl]-amide;
3-Amino-5-difluoromethyl-pyridine-2-carboxylic acid [3-((R)-5-amino-6,6-bis-fluoromethyl-3-methyl-3,6-dihydro-2H-[1,4]oxazin-3-yl)-4-fluoro-phenyl]-amide;
3-Amino-5-cyano-pyridine-2-carboxylic acid [3-((R)-5-amino-6,6-bis-fluoromethyl-3-methyl-3,6-dihydro-2H-[1,4]oxazin-3-yl)-4-fluoro-phenyl]-amide;
5-Difluoromethyl-3-methyl-pyridine-2-carboxylic acid [3-((R)-5-amino-3,6,6-tris-fluoromethyl-3,6-dihydro-2H-[1,4]oxazin-3-yl)-4-fluoro-phenyl]-amide;
3-Chloro-5-difluoromethyl-pyridine-2-carboxylic acid [3-((R)-5-amino-3,6,6-tris-fluoromethyl-3,6-dihydro-2H-[1,4]oxazin-3-yl)-4-fluoro-phenyl]-amide;
3-Chloro-5-trifluoromethyl-pyridine-2-carboxylic acid [3-((R)-5-amino-3,6,6-tris-fluoromethyl-3,6-dihydro-2H-[1,4]oxazin-3-yl)-4-fluoro-phenyl]-amide;
3,5-Dichloro-pyridine-2-carboxylic acid [3-((R)-5-amino-3,6,6-tris-fluoromethyl-3,6-dihydro-2H-[1,4]oxazin-3-yl)-4-fluoro-phenyl]-amide;
3-Amino-5-cyano-pyridine-2-carboxylic acid [3-((R)-5-amino-3,6,6-tris-fluoromethyl-3,6-dihydro-2H-[1,4]oxazin-3-yl)-4-fluoro-phenyl]-amide;
3-Chloro-5-cyano-pyridine-2-carboxylic acid [3-((R)-5-amino-3,6,6-tris-fluoromethyl-3,6-dihydro-2H-[1,4]oxazin-3-yl)-4-fluoro-phenyl]-amide;
N-[3-((3R,6R)-5-Amino-3,6-dimethyl-6-trifluoromethyl-3,6-dihydro-2H-[1,4]oxazin-3-yl)-4-fluoro-phenyl]-6-methoxy-2-methyl-nicotinamide;
N-[3-((3R,6R)-5-Amino-3,6-dimethyl-6-trifluoromethyl-3,6-dihydro-2H-[1,4]oxazin-3-yl)-4-fluoro-phenyl]-6-trideuteromethoxy-2-methyl-nicotinamide;
2-Amino-N-[3-((3R,6R)-5-amino-3,6-dimethyl-6-trifluoromethyl-3,6-dihydro-2H-[1,4]oxazin-3-yl)-4-fluoro-phenyl]-6-ethoxy-nicotinamide;
2-Amino-N-[3-((3R,6R)-5-amino-3,6-dimethyl-6-trifluoromethyl-3,6-dihydro-2H-[1,4]oxazin-3-yl)-4-fluoro-phenyl]-6-methoxy-nicotinamide;
2-Amino-N-[3-((3R,6R)-5-amino-3,6-dimethyl-6-trifluoromethyl-3,6-dihydro-2H-[1,4]oxazin-3-yl)-4-fluoro-phenyl]-6-trideuteromethoxy-nicotinamide;
2-Amino-N-[3-((3R,6R)-5-amino-3,6-dimethyl-6-trifluoromethyl-3,6-dihydro-2H-[1,4]oxazin-3-yl)-4-fluoro-phenyl]-6-pentadeuteroethoxy-nicotinamide;
N-[3-((3R,6R)-5-Amino-3,6-dimethyl-6-trifluoromethyl-3,6-dihydro-2H-[1,4]oxazin-3-yl)-4-fluoro-phenyl]-2-chloro-6-methoxy-nicotinamide;
N-[3-((3R,6R)-5-Amino-3,6-dimethyl-6-trifluoromethyl-3,6-dihydro-2H-[1,4]oxazin-3-yl)-4-fluoro-phenyl]-2-chloro-6-ethoxy-nicotinamide;
2-Amino-N-[3-((3R,6R)-5-amino-3,6-dimethyl-6-trifluoromethyl-3,6-dihydro-2H-[1,4]oxazin-3-yl)-4-fluoro-phenyl]-6-cyclopropylmethoxy-nicotinamide; and
2-Amino-N-[3-((3R,6R)-5-amino-3,6-dimethyl-6-trifluoromethyl-3,6-dihydro-2H-[1,4]oxazin-3-yl)-4-fluoro-phenyl]-6-(2,2,2-trifluoro-ethoxy)-nicotinamide.

In certain embodiments, the invention relates to a method of treating diseases associated with inhibition of BACE-2 activity comprising administering to a subject a therapeutically effective amount of a compound of the formula (II), (II′) and (II″), or a pharmaceutically acceptable salt thereof, wherein:

(1) R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
(2) R₁ is hydrogen, cyano, halogen, (C₁₋₄)alkyl, halogen-(C₁₋₄)alkyl, (C₁₋₄)alkoxy, or halogen-(C₁₋₄)alkoxy;
(3) R₁ is hydrogen;
(4) R₂ₐ is a (C₃₋₈)cycloalkyl, aryl, heteroaryl or non-aromatic heterocyclyl group G₁, which group G₁ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxyl-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₂, which group G₂ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
(5) R₂ₐ is a (C₃₋₈)cycloalkyl, aryl, heteroaryl or non-aromatic heterocyclyl group G₁, which group G₁ is optionally substituted by 1 to 4 substituents independently selected from the group, consisting of cyano, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₂, which group G₂ is optionally substituted by 1 to 4 substituents independently selected from the group, consisting of cyano, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
(6) R₂ₐ is a (C₃₋₈)cycloalkyl, aryl or heteroaryl group G₁, which group G₁ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl or heteroaryl group G₂, which group G₂ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
(7) R₂ₐ is a heteroaryl group G₁, which group G₁ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl or heteroaryl group G₂, which group G₂ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
(8) R₂ₐ is a heteroaryl group G₁, which group G₁ is optionally substituted by 1 or 2 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl or heteroaryl group G₂, which group G₂ is unsubstituted;
(9) R₂ₐ is an aryl or heteroaryl group G₁, which group G₁ is optionally substituted by 1, 2, 3 or 4 substituents independently selected from the group, consisting of cyano, amino, aminocarbonyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, and (C₂₋₈)alkynoxy;
(10) R₂ₐ is phenyl or a 5- or 6-membered heteroaryl group G₁ in which structure 1, 2, 3, or 4 ring members are hetero ring members independently selected from the group consisting of a nitrogen ring member, an oxygen ring member and a sulfur ring member, which group G₁ is optionally substituted by 1, 2, 3 or 4 substituents independently selected from the group, consisting of cyano, amino, aminocarbonyl, halogen, (C₁₋₄)alkyl, halogen-(C₁₋₄)alkyl, hydroxy, oxo, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, (C₂₋₄)alkynyl, (C₂₋₄)alkenoxy, and (C₂₋₄)alkynoxy;
(11) R₂ₐ is a 6-membered heteroaryl group G₁ in which structure 1, 2, 3, or 4 ring members are hetero ring members independently selected from the group consisting of a nitrogen ring member, an oxygen ring member and a sulfur ring member, which group G₁ is optionally substituted by 1, 2, 3 or 4 substituents independently selected from the group, consisting of cyano, amino, aminocarbonyl, halogen, (C₁₋₄)alkyl, halogen-(C₁₋₄)alkyl, hydroxy, oxo, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, (C₂₋₄)alkynyl, (C₂₋₄)alkenoxy, and (C₂₋₄)alkynoxy;
(12) R₂ₐ is a 6-membered heteroaryl group G₁ in which structure 1, 2, 3, or 4 ring members are hetero ring members independently selected from the group consisting of a nitrogen ring member, an oxygen ring member and a sulfur ring member, which group G₁ is optionally substituted by 1, 2, 3 or 4 substituents independently selected from the group, consisting of cyano, halogen, (C₁₋₄)alkyl, halogen-(C₁₋₄)alkyl, hydroxy, oxo, (C₁₋₄)alkoxy and halogen-(C₁₋₄)alkoxy;
(13) R₂ₐ is a pyridyl or pyrazinyl group which is optionally substituted by 1, 2 or 3 substituents independently selected from the group, consisting of cyano, amino, aminocarbonyl, halogen, (C₁₋₄)alkyl, halogen-(C₁₋₄)alkyl, hydroxy, oxo, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, (C₂₋₄)alkynyl, (C₂₋₄)alkenoxy, and (C₂₋₄)alkynoxy;
(14) R₂ₐ is a pyridyl or pyrazinyl group which is optionally substituted by 1, 2 or 3 substituents independently selected from the group, consisting of cyano, halogen, (C₁₋₄)alkyl, halogen-(C₁₋₄)alkyl, hydroxy, oxo, (C₁₋₄)alkoxy and halogen-(C₁₋₄)alkoxy;
(15) R₂ₐ is a pyridin-2-yl or pyrazin-2-yl group which is optionally substituted by 1, 2 or 3 substituents independently selected from the group, consisting of cyano, amino, aminocarbonyl, halogen, (C₁₋₄)alkyl, halogen-(C₁₋₄)alkyl, hydroxy, oxo, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, (C₂₋₄)alkynyl, (C₂₋₄)alkenoxy, and (C₂₋₄)alkynoxy;
(16) R₂ₐ is a pyridin-2-yl or pyrazin-2-yl group which is optionally substituted by 1, 2 or 3 substituents independently selected from the group, consisting of cyano, halogen, (C₁₋₄)alkyl, halogen-(C₁₋₄)alkyl, hydroxy, oxo, (C₁₋₄)alkoxy and halogen-(C₁₋₄)alkoxy;
(17) R₂ₐ is a pyridin-2-yl or pyrazin-2-yl group which is optionally substituted by 1 or 2 substituents independently selected from the group, consisting of cyano, amino, fluoro, bromo, chloro, hydroxyl, oxo, methyl and difluoromethoxy;
(18) R₂ₐ is a pyridyl or pyrazinyl group which is substituted by 1, 2 or 3 substituents and wherein one of the substituents is located at the para position of the pyridyl or pyrazinyl group relative to the amide linker and wherein the substituents are independently selected from the group, consisting of cyano, amino, aminocarbonyl, halogen, (C₁₋₄)alkyl, halogen-(C₁₋₄)alkyl, hydroxy, oxo, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, (C₂₋₄)alkynyl, (C₂₋₄)alkenoxy, and (C₂₋₄)alkynoxy;
(19) R₂ₐ is a pyridyl or pyrazinyl group which is substituted by 1, 2 or 3 substituents and wherein one of the substituents is located at the para position of the pyridyl or pyrazinyl group relative to the amide linker and wherein the substituents are independently selected from the group, consisting of cyano, halogen, (C₁₋₄)alkyl, halogen-(C₁₋₄)alkyl, hydroxy, oxo, (C₁₋₄)alkoxy and halogen-(C₁₋₄)alkoxy;
(20) R₂ₐ is a pyridin-2-yl or pyrazin-2-yl group which is substituted by 1, 2 or 3 substituents and wherein one of the substituents is located at the para position of the pyridin-2-yl or pyrazin-2-yl group relative to the amide linker and wherein the substituents are independently selected from the group, consisting of cyano, halogen, (C₁₋₄)alkyl, halogen-(C₁₋₄)alkyl, hydroxy, oxo, (C₁₋₄)alkoxy and halogen-(C₁₋₄)alkoxy;
(21) R₂ₐ is a pyridyl or pyrazinyl group which is substituted by 2 or 3 substituents and wherein one of the substituents is located at the para position and one of the substituents is located at the ortho position of the pyridyl or pyrazinyl group relative to the amide linker and wherein the substituents are independently selected from the group, consisting of cyano, amino, aminocarbonyl, halogen, (C₁₋₄)alkyl, halogen-(C₁₋₄)alkyl, hydroxy, oxo, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, (C₂₋₄)alkynyl, (C₂₋₄)alkenoxy, and (C₂₋₄)alkynoxy;
(22) R₂ₐ is a pyridyl or pyrazinyl group which is substituted by 2 or 3 substituents and wherein one of the substituents is located at the para position and one of the substituents is located at the ortho position of the pyridyl or pyrazinyl group relative to the amide linker and wherein the substituents are independently selected from the group, consisting of cyano, halogen, (C₁₋₄)alkyl, halogen-(C₁₋₄)alkyl, hydroxy, oxo, (C₁₋₄)alkoxy and halogen-(C₁₋₄)alkoxy;
(23) R₂ₐ is a pyridin-2-yl or pyrazin-2-yl group which is substituted by 2 substituents and wherein one of the substituents is located at the para position and one of the substituents is located at the ortho position of the pyridin-2-yl or pyrazin-2-yl group relative to the amide linker and wherein the substituents are independently selected from the group, consisting of cyano, halogen, (C₁₋₄)alkyl, halogen-(C₁₋₄)alkyl, hydroxy, oxo, (C₁₋₄)alkoxy and halogen-(C₁₋₄)alkoxy;
(24) R₂ₐ is a pyridin-2-yl or pyrazin-2-yl group which is substituted by 2 substituents and wherein one of the substituents is located at the para position and one of the substituents is located at the ortho position of the pyridin-2-yl or pyrazin-2-yl group relative to the amide linker and wherein the substituents are independently selected from the group, consisting of cyano, amino, fluoro, bromo, chloro, hydroxyl, oxo, methyl and difluoromethoxy;
(25) R₃ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
(26) R₃ is hydrogen, cyano, halogen, (C₁₋₄)alkyl, halogen-(C₁₋₄)alkyl, (C₁₋₄)alkoxy, or halogen-(C₁₋₄)alkoxy;
(27) R₃ is hydrogen;
(28) either
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl; and
R₅ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
(29) R₄ is hydrogen, cyano, halogen, (C₁₋₄)alkyl, halogen-(C₁₋₄)alkyl, (C₁₋₄)alkoxy, or halogen-(C₁₋₄)alkoxy;
(30) R₅ is hydrogen, cyano, halogen, (C₁₋₄)alkyl, halogen-(C₁₋₄)alkyl, (C₁₋₄)alkoxy, or halogen-(C₁₋₄)alkoxy;
(31) R₄ is hydrogen, or halogen; and
R₅ is hydrogen, or halogen;
(32) R₄ is hydrogen; and
R₅ is halogen;
(33) R₄ is hydrogen; and
R₅ is fluoro;
(34) R₄ is hydrogen; and
R₅ is hydrogen or fluoro;
(35) R₄ is halogen; and
R₅ is hydrogen;
(36) each of R₄ and R₅ is hydrogen;
(37) R₆ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₈)alkylamino-(C₁₋₈)alkyl, N,N-di-[(C₁₋₈)alkyl]amino-(C₁₋₈)alkyl with two identical or different (C₁₋₈)alkyl moieties in the N,N-di-[(C₁₋₈)alkyl]amino moiety, (C₂₋₈)alkenyl, or (C₂₋₈)alkynyl;
(38) R₆ is (C₁₋₈)alkyl, or halogen-(C₁₋₈)alkyl;
(39) R₆ is (C₁₋₃)alkyl, or halogen-(C₁₋₃)alkyl;
(40) R₆ is (C₁₋₈)alkyl, or fluorine-substituted (C₁₋₈)alkyl;
(41) R₆ is (C₁₋₃)alkyl, or fluorine-substituted (C₁₋₃)alkyl;
(42) R₆ is methyl, fluoromethyl, or difluoromethyl;
(43) R₂₀ is hydrogen, (C₁₋₈)alkyl, (C₁₋₈)alkyl substituted by halogen, (C₃₋₈)cycloalkyl-(C₁₋₈)alkyl, (C₃₋₈)cycloalkoxy-(C₁₋₈)alkyl, aryloxy-(C₁₋₈)alkyl, (C₁₋₈)alkoxy-(C₁₋₈)alkyl, (C₁₋₈)alkylthio-(C₁₋₈)alkyl, (C₁₋₈)alkylsulfinyl, (C₁₋₈)alkylsulfinyl-(C₁₋₈)alkyl, (C₁₋₈)alkylsulfonyl, (C₁₋₈)alkylsulfonyl-(C₁₋₈)alkyl, amino-(C₁₋₈)alkyl, (C₁₋₈)alkylamino-(C₁₋₈)alkyl, di(C₁₋₈)alkylamino-(C₁₋₈)alkyl with two identical or different (C₁₋₈)alkyl moieties in the di(C₁₋₈)alkylamino moiety, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyl-(C₁₋₈)alkyl, (C₁₋₈)alkylcarbonyl-(C₁₋₈)alkyl, (C₁₋₈)alkoxycarbonyl, (C₁₋₈)alkoxycarbonyl-(C₁₋₈)alkyl, or a (C₃₋₈)cycloalkylcarbonyl, arylcarbonyl, aryl-(C₁₋₈)alkylcarbonyl, heteroarylcarbonyl, heteroaryl-(C₁₋₈)alkylcarbonyl, non-aromatic heterocyclylcarbonyl, (C₃₋₈)cycloalkylsulfonyl, arylsulfonyl, aryl-(C₁₋₈)alkylsulfonyl, heteroarylsulfonyl, heteroaryl-(C₁₋₈)alkylsulfonyl, non-aromatic heterocyclylsulfonyl, (C₃₋₈)cycloalkyl, aryl, aryl-(C₁₋₈)alkyl, heteroaryl, heteroaryl-(C₁₋₈)alkyl or non-aromatic heterocyclyl group G₃, which group G₃ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₄, which group G₄ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
(44) R₂₀ is (C₁₋₈)alkyl, (C₁₋₈)alkoxy-(C₁₋₈)alkyl, or a heteroaryl group G₃, which group G₃ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₄, which group G₄ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
(45) R₂₀ is hydrogen, (C₁₋₆)alkyl, halogen-(C₁₋₆)alkyl, (C₁₋₄)alkoxy-(C₁₋₄)alkyl, (C₁₋₆)alkylcarbonyl, (C₁₋₆)alkoxycarbonyl, halogen-(C₁₋₆)alkoxycarbonyl, (C₁₋₆)alkoxy-(C₁₋₆)alkylcarbonyl, (C₃₋₆)cycloalkyl, (C₃₋₆)cycloalkyl-carbonyl, or a heteroaryl group optionally substituted by 1, 2, or 3 substituents independently selected from the group consisting of cyano, halogen, hydroxyl, (C₁₋₄)alkyl, halogen-(C₁₋₄)alkyl, (C₁₋₄)alkoxy, halogen-(C₁₋₄)alkoxy, (C₁₋₃)alkoxy-(C₁₋₃)alkyl and (C₁₋₃)alkoxy-(C₁₋₃)alkoxy;
(46) R₂₀ is hydrogen, methyl, ethyl, isopropyl, acetyl, methoxyethyl, methoxycarbonyl, dichloroethoxycarbonyl, methoxymethylcarbonyl, cyclopropylcarbonyl, pyridinyl, or methyl substituted pyrazolyl;

### (47) E₁ is —C(R₇)(R₈)—, or —C(R₇)(R₈)—C(R₉)(R₁₀)—;

### (48) E₁ is —C(R₇)(R₈)—;

### (49) E₂ₐ is —C(R₁₁ₐ)(R₁₂ₐ)—, or —C(R₁₁ₐ)(R₁₂ₐ)—C(R₁₃)(R₁₄)—;

### (50) E₂ is —C(R₁₂)(R₁₃)—;

(51) either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
(52) either
each of R₇ and R₈ is independently selected from the group, consisting of hydrogen, cyano, halogen, (C₁₋₃)alkyl and halogen-(C₁₋₃)alkyl;
or
R₇ and R₈, taken together, are oxo or —CH₂—CH₂—;
(53) either
each of R₇ and R₈ is hydrogen;
or
R₇ and R₈, taken together, are oxo;
(54) each of R₇ and R₈ is hydrogen;
(55) either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
(56) each of R₉ and R₁₀ is hydrogen;
(57) either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CH₂—CH₂—;
(58) each of R₁₁ₐ and R₁₂ₐ is independently selected from the group, consisting of hydrogen, halogen, (C₁₋₈)alkyl and halogen-(C₁₋₈)alkyl;
(59) each of R₁₁ₐ and R₁₂ₐ is independently selected from the group, consisting of hydrogen, (C₁₋₈)alkyl and halogen-(C₁₋₈)alkyl;
(60) either
each of R₁₁ₐ and R₁₂ₐ is independently selected from the group, consisting of hydrogen, cyano, halogen, (C₁₋₃)alkyl and halogen-(C₁₋₃)alkyl;
or
R₁₁ₐ and R₁₂ₐ, taken together, are oxo or —CR₁₆R₁₇—CR₁₈R₁₉—
wherein R₁₆, R₁₇, R₁₈ and R₁₉ are independently selected from hydrogen and fluoro;
(61) either
each of R₁₁ₐ and R₁₂ₐ is independently selected from the group, consisting of hydrogen, (C₁₋₃)alkyl and halogen-(C₁₋₃)alkyl;
or
R₁₁ₐ and R₁₂ₐ, taken together, are oxo;
(62) either
each of R₁₁ₐ and R₁₂ₐ is independently selected from the group, consisting of hydrogen, methyl and ethyl;
or
R₁₁ₐ and R₁₂ₐ, taken together, are oxo;
(63) R₁₁ₐ is (C₁₋₈)alkyl, and R₁₂ₐ is halogen-(C₁₋₈)alkyl;
(64) R₁₁ₐ is (C₁₋₃)alkyl, and R₁₂ₐ is halogen-(C₁₋₃)alkyl;
(65) each of R₁₁ₐ and R₁₂ₐ is hydrogen;
(66) R₁₁ₐ and R₁₂ₐ, taken together, are oxo;
(67) either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—;
(68) each of R₁₃ and R₁₄ is hydrogen.

The skilled person would understand that the embodiments (1) to (68) may be used independently, collectively or in any combination or sub-combination to the limit the scope of the invention as described hereinbefore in relation to compounds of the formula (II), (II′) and (II″).

In one embodiment, the invention relates to a method of treating diseases associated with inhibition of BACE-2 activity comprising administering to a subject a therapeutically effective amount of a compound of the formula (IIa):

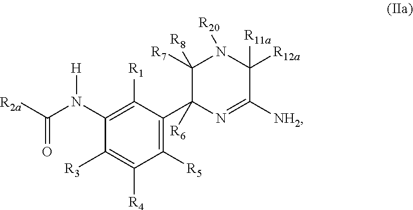

or a pharmaceutically acceptable salt thereof, wherein:

R₁ is hydrogen, cyano, halogen, (C₁₋₄)alkyl, halogen-(C₁₋₄)alkyl, (C₁₋₄)alkoxy, or halogen-(C₁₋₄)alkoxy;
R₂ₐ is phenyl or a 5- or 6-membered heteroaryl group G₁ in which structure 1, 2, 3, or 4 ring members are hetero ring members independently selected from the group consisting of a nitrogen ring member, an oxygen ring member and a sulfur ring member, which group G₁ is optionally substituted by 1, 2, 3 or 4 substituents independently selected from the group, consisting of cyano, amino, aminocarbonyl, halogen, (C₁₋₄)alkyl, halogen-(C₁₋₄)alkyl, hydroxy, oxo, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, (C₂₋₄)alkynyl, (C₂₋₄)alkenoxy, and (C₂₋₄)alkynoxy;
R₃, R₄ and R₅ are independently selected from the group consisting of hydrogen, cyano, halogen, (C₁₋₄)alkyl, halogen-(C₁₋₄)alkyl, (C₁₋₄)alkoxy, or halogen-(C₁₋₄)alkoxy;
R₆ is (C₁₋₃)alkyl, or fluorine-substituted (C₁₋₃)alkyl;
R₂₀ is hydrogen, (C₁₋₆)alkyl, halogen-(C₁₋₆)alkyl, (C₁₋₄)alkoxy-(C₁₋₄)alkyl, (C₁₋₆)alkylcarbonyl, (C₁₋₆)alkoxycarbonyl, halogen-(C₁₋₆)alkoxycarbonyl, (C₁₋₆)alkoxy-(C₁₋₆)alkylcarbonyl, (C₃₋₆)cycloalkyl, (C₃₋₆)cycloalkyl-carbonyl, or a heteroaryl group optionally substituted by 1, 2, or 3 substituents independently selected from the group consisting of cyano, halogen, hydroxyl, (C₁₋₄)alkyl, halogen-(C₁₋₄)alkyl, (C₁₋₄)alkoxy, halogen-(C₁₋₄)alkoxy, (C₁₋₃)alkoxy-(C₁₋₃)alkyl and (C₁₋₃)alkoxy-(C₁₋₃)alkoxy;
   either
each of R₇ and R₈ is independently selected from the group, consisting of hydrogen, cyano, halogen, (C₁₋₃)alkyl and halogen-(C₁₋₃)alkyl;
or
R₇ and R₈, taken together, are oxo or —CH₂—CH₂—; and
either
each of R₁₁ and R₁₂ₐ is independently selected from the group, consisting of hydrogen, cyano, halogen, (C₁₋₃)alkyl and halogen-(C₁₋₃)alkyl;
or

R₁₁ₐ and R₁₂ₐ, taken together, are oxo or —CR₁₆R₁₇—CR₁₈R₁₉—
wherein R₁₆, R₁₇, R₁₈ and R₁₉ are independently selected from hydrogen and fluoro.

In another embodiment, the invention relates to a method of treating diseases associated with inhibition of BACE-2 activity comprising administering to a subject a therapeutically effective amount of a compound of the formula (IIb):

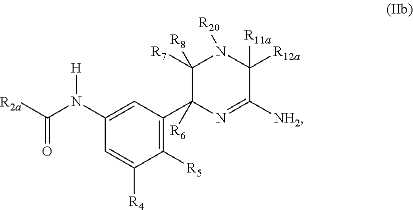

or a pharmaceutically acceptable salt thereof, wherein:
R₂ₐ is a 6-membered heteroaryl group G₁ in which structure 1, 2, 3, or 4 ring members are hetero ring members independently selected from the group consisting of a nitrogen ring member, an oxygen ring member and a sulfur ring member, which group G₁ is optionally substituted by 1, 2, 3 or 4 substituents independently selected from the group, consisting of cyano, amino, aminocarbonyl, halogen, (C₁₋₄)alkyl, halogen-(C₁₋₄)alkyl, hydroxy, oxo, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, (C₂₋₄)alkynyl, (C₂₋₄)alkenoxy, and (C₂₋₄)alkynoxy;
R₄ and R₅ are independently hydrogen, or halogen;
R₆ is (C₁₋₃)alkyl, or fluorine-substituted (C₁₋₃)alkyl;
R₂₀ is hydrogen, (C₁₋₆)alkyl, halogen-(C₁₋₆)alkyl, (C₁₋₄)alkoxy-(C₁₋₄)alkyl, (C₁₋₆)alkylcarbonyl, (C₁₋₆)alkoxycarbonyl, halogen-(C₁₋₆)alkoxycarbonyl, (C₁₋₆)alkoxy-(C₁₋₆)alkylcarbonyl, (C₃₋₆)cycloalkyl, (C₃₋₆)cycloalkyl-carbonyl, or a heteroaryl group optionally substituted by 1, 2, or 3 substituents independently selected from the group consisting of cyano, halogen, hydroxyl, (C₁₋₄)alkyl, halogen-(C₁₋₄)alkyl, (C₁₋₄)alkoxy, halogen-(C₁₋₄)alkoxy, (C₁₋₃)alkoxy-(C₁₋₃)alkyl and (C₁₋₃)alkoxy-(C₁₋₃)alkoxy;
either
each of R₇ and R₈ is hydrogen;
or
R₇ and R₈, taken together, are oxo; and
either
each of R₁₁ₐ and R₁₂ₐ is independently selected from the group, consisting of hydrogen, (C₁₋₃)alkyl and halogen-(C₁₋₃)alkyl;
or
R₁₁ₐ and R₁₂ₐ, taken together, are oxo.

In another embodiment, the invention relates to a method of treating diseases associated with inhibition of BACE-2 activity comprising administering to a subject a therapeutically effective amount of a compound of the formula (IIc):

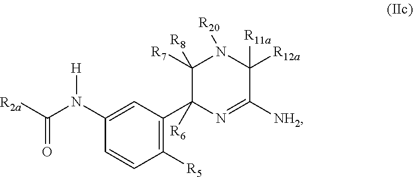

or a pharmaceutically acceptable salt thereof, wherein:
R₂ₐ is a pyridyl or pyrazinyl group which is substituted by 1, 2 or 3 substituents and wherein one of the substituents is located at the para position of the pyridyl or pyrazinyl group relative to the amide linker and wherein the substituents are independently selected from the group, consisting of cyano, amino, aminocarbonyl, halogen, (C₁₋₄)alkyl, halogen-(C₁₋₄)alkyl, hydroxy, oxo, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, (C₂₋₄)alkynyl, (C₂₋₄)alkenoxy, and (C₂₋₄)alkynoxy;
R₅ is hydrogen or fluoro;
R₆ is methyl, fluoromethyl, or difluoromethyl;
R₂₀ is hydrogen, (C₁₋₆)alkyl, halogen-(C₁₋₆)alkyl, (C₁₋₄)alkoxy-(C₁₋₄)alkyl, (C₁₋₆)alkylcarbonyl, (C₁₋₆)alkoxycarbonyl, halogen-(C₁₋₆)alkoxycarbonyl, (C₁₋₆)alkoxy-(C₁₋₆)alkylcarbonyl, (C₃₋₆)cycloalkyl, (C₃₋₆)cycloalkyl-carbonyl, or a heteroaryl group optionally substituted by 1, 2, or 3 substituents independently selected from the group consisting of cyano, halogen, hydroxyl, (C₁₋₄)alkyl, halogen-(C₁₋₄)alkyl, (C₁₋₄)alkoxy, halogen-(C₁₋₄)alkoxy, (C₁₋₃)alkoxy-(C₁₋₃)alkyl and (C₁₋₃)alkoxy-(C₁₋₃)alkoxy;
either
each of R₇ and R₈ is hydrogen;
or
R₇ and R₈, taken together, are oxo; and
either
each of R₁₁ₐ and R₁₂ₐ is independently selected from the group, consisting of hydrogen, (C₁₋₃)alkyl and halogen-(C₁₋₃)alkyl;
or
R₁₁ₐ and R₁₂ₐ, taken together, are oxo.

In another embodiment, the invention relates to a method of treating diseases associated with inhibition of BACE-2 activity comprising administering to a subject a therapeutically effective amount of a compound of the formula (IId):

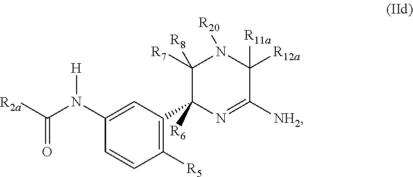

or a pharmaceutically acceptable salt thereof, wherein:
R₂ₐ is a pyridyl or pyrazinyl group which is substituted by 1, 2 or 3 substituents and wherein one of the substituents is located at the para position of the pyridyl or pyrazinyl group relative to the amide linker and wherein the substituents are independently selected from the group, consisting of cyano, halogen, (C₁₋₄)alkyl, halogen-(C₁₋₄)alkyl, hydroxy, oxo, (C₁₋₄)alkoxy and halogen-(C₁₋₄)alkoxy;
R₅ is hydrogen or fluoro;
R₆ is methyl, fluoromethyl, or difluoromethyl;
R₂₀ is hydrogen, (C₁₋₆)alkyl, halogen-(C₁₋₆)alkyl, (C₁₋₄)alkoxy-(C₁₋₄)alkyl, (C₁₋₆)alkylcarbonyl, (C₁₋₆)alkoxycarbonyl, halogen-(C₁₋₆)alkoxycarbonyl, (C₁₋₆)alkoxy-(C₁₋₆)alkylcarbonyl, (C₃₋₆)cycloalkyl, (C₃₋₆)cycloalkyl-carbonyl, or a heteroaryl group optionally substituted by 1, 2, or 3 substituents independently selected from the group consisting of cyano, halogen, hydroxyl, (C₁₋₄)alkyl, halogen-(C₁₋₄)alkyl, (C₁₋₄)alkoxy, halogen-(C₁₋₄)alkoxy, (C₁₋₃)alkoxy-(C₁₋₃)alkyl and (C₁₋₃)alkoxy-(C₁₋₃)alkoxy;
either
each of R₇ and R₈ is hydrogen;
or
R₇ and R₈, taken together, are oxo; and
either
each of R₁₁ₐ and R₁₂ₐ is independently selected from the group, consisting of hydrogen, (C₁₋₃)alkyl and halogen-(C₁₋₃)alkyl;
or
R₁₁ₐ and R₁₂ₐ, taken together, are oxo.

In another embodiment, the invention relates to a method of treating diseases associated with inhibition of BACE-2 activity comprising administering to a subject a therapeutically effective amount of a compound which is selected from:
5-Bromo-pyridine-2-carboxylic acid [3-(6-amino-2,4-dimethyl-3-oxo-2,3,4,5-tetrahydro-pyrazin-2-yl)-4-fluoro-phenyl]-amide;
5-Bromo-pyridine-2-carboxylic acid [3-(6-amino-2,4-dimethyl-2,3,4,5-tetrahydro-pyrazin-2-yl)-4-fluoro-phenyl]-amide;
5-Bromo-pyridine-2-carboxylic acid [3-(6-amino-2,4-dimethyl-3-oxo-2,3,4,5-tetrahydro-pyrazin-2-yl)-phenyl]-amide;
5-Bromo-pyridine-2-carboxylic acid [3-(6-amino-2,4-dimethyl-5-oxo-2,3,4,5-tetrahydro-pyrazin-2-yl)-4-fluoro-phenyl]-amide;
5-Chloro-pyridine-2-carboxylic acid [3-(6-amino-2,4-dimethyl-5-oxo-2,3,4,5-tetrahydro-pyrazin-2-yl)-4-fluoro-phenyl]-amide;
5-Bromo-pyridine-2-carboxylic acid [3-(6-amino-4-ethyl-2-methyl-5-oxo-2,3,4,5-tetrahydro-pyrazin-2-yl)-4-fluoro-phenyl]-amide;
3,5-Dichloro-pyridine-2-carboxylic acid [3-(6-amino-2,4-dimethyl-5-oxo-2,3,4,5-tetrahydro-pyrazin-2-yl)-4-fluoro-phenyl]-amide;
3,5-Dichloro-pyridine-2-carboxylic acid [3-(6-amino-4-ethyl-2-methyl-5-oxo-2,3,4,5-tetrahydro-pyrazin-2-yl)-4-fluoro-phenyl]-amide;
5-Bromo-pyridine-2-carboxylic acid [3-(6-amino-4-isopropyl-2-methyl-5-oxo-2,3,4,5-tetrahydro-pyrazin-2-yl)-4-fluoro-phenyl]-amide;
3,5-Dichloro-pyridine-2-carboxylic acid {3-[6-amino-4-(2-methoxy-ethyl)-2-methyl-5-oxo-2,3,4,5-tetrahydro-pyrazin-2-yl]-4-fluoro-phenyl}-amide;
5-Bromo-pyridine-2-carboxylic acid {3-[6-amino-4-(2-methoxy-ethyl)-2-methyl-5-oxo-2,3,4,5-tetrahydro-pyrazin-2-yl]-4-fluoro-phenyl}-amide;
5-Bromo-pyridine-2-carboxylic acid {3-[6-amino-2-methyl-4-(1-methyl-1H-pyrazol-4-yl)-5-oxo-2,3,4,5-tetrahydro-pyrazin-2-yl]-4-fluoro-phenyl}-amide;
5-Bromo-pyridine-2-carboxylic acid [3-(6-amino-2-methyl-5-oxo-4-pyridin-3-yl-2,3,4,5-tetrahydro-pyrazin-2-yl)-4-fluoro-phenyl]-amide;
5-Cyano-pyridine-2-carboxylic acid [3-(6-amino-4-ethyl-2-methyl-5-oxo-2,3,4,5-tetrahydro-pyrazin-2-yl)-4-fluoro-phenyl]-amide;
5-Bromo-pyridine-2-carboxylic acid [3-(6-amino-5-ethyl-2,4-dimethyl-3-oxo-2,3,4,5-tetrahydro-pyrazin-2-yl)-phenyl]-amide;
5-Bromo-pyridine-2-carboxylic acid [3-(6-amino-2-difluoromethyl-2,3,4,5-tetrahydro-pyrazin-2-yl)-4-fluoro-phenyl]-amide;
5-Cyano-pyridine-2-carboxylic acid [3-(6-amino-2-difluoromethyl-2,3,4,5-tetrahydro-pyrazin-2-yl)-4-fluoro-phenyl]-amide;
5-Cyano-pyridine-2-carboxylic acid [3-(6-amino-2-difluoromethyl-5-oxo-2,3,4,5-tetrahydro-pyrazin-2-yl)-4-fluoro-phenyl]-amide;
5-Amino-3-{5-[(5-bromo-pyridine-2-carbonyl)-amino]-2-fluoro-phenyl}-3-difluoromethyl-3,6-dihydro-2H-pyrazine-1-carboxylic acid methyl ester;
5-Amino-3-{5-[(5-cyano-pyridine-2-carbonyl)-amino]-2-fluoro-phenyl}-3-difluoromethyl-3,6-dihydro-2H-pyrazine-1-carboxylic acid methyl ester;
5-Bromo-3-methyl-pyridine-2-carboxylic acid [3-(4-acetyl-6-amino-2-difluoromethyl-2,3,4,5-tetrahydro-pyrazin-2-yl)-4-fluoro-phenyl]-amide;
5-Amino-3-{5-[(5-bromo-3-methyl-pyridine-2-carbonyl)-amino]-2-fluoro-phenyl}-3-difluoromethyl-3,6-dihydro-2H-pyrazine-1-carboxylic acid 2,2-dichloro-ethyl ester;
5-Bromo-3-methyl-pyridine-2-carboxylic acid {3-[6-amino-2-difluoromethyl-4-(2-methoxy-acetyl)-2,3,4,5-tetrahydro-pyrazin-2-yl]-4-fluoro-phenyl}-amide;
5-Bromo-3-methyl-pyridine-2-carboxylic acid [3-(6-amino-4-cyclopropanecarbonyl-2-difluoromethyl-2,3,4,5-tetrahydro-pyrazin-2-yl)-4-fluoro-phenyl]-amide;
5-Bromo-3-methyl-pyridine-2-carboxylic acid [3-(6-amino-2-difluoromethyl-2,3,4,5-tetrahydro-pyrazin-2-yl)-4-fluoro-phenyl]-amide;
5-Difluoromethoxy-3-methyl-pyridine-2-carboxylic acid [3-(4-acetyl-6-amino-2-difluoromethyl-2,3,4,5-tetrahydro-pyrazin-2-yl)-4-fluoro-phenyl]-amide;
5-Difluoromethoxy-3-methyl-pyridine-2-carboxylic acid [3-(6-amino-2-difluoromethyl-2,3,4,5-tetrahydro-pyrazin-2-yl)-4-fluoro-phenyl]-amide;
3-Amino-5-methoxy-pyrazine-2-carboxylic acid [3-(6-amino-2-difluoromethyl-2,3,4,5-tetrahydro-pyrazin-2-yl)-4-fluoro-phenyl]-amide; and
3-Amino-5-oxo-4,5-dihydro-pyrazine-2-carboxylic acid [3-(6-amino-2-difluoromethyl-2,3,4,5-tetrahydro-pyrazin-2-yl)-4-fluoro-phenyl]-amide,
   or a pharmaceutically acceptable salt thereof.

In another embodiment, the invention relates to a method of treating diseases associated with inhibition of BACE-2 activity comprising administering to a subject a therapeutically effective amount of a compound of the formula (III), (III′), or (III″), or a pharmaceutically acceptable salt thereof, in which:

### (1) X₁ is CR₁ or N;

X₃ is CR₃ or N;
X₄ is CR₄ or N;
X₅ is CR₅ₐ;
wherein at least one of X₁, X₃ and X₄ is N and not more than 2 of X₁, X₃ and X₄ are N.

### (2) X₁ is CH or N;

X₃ is CH or N;
X₄ is CR₄ or N;
X₅ is CR₅ₐ;
wherein one and not more than one of X₁, X₃ and X₄ is N;

### (3) X₁ is N; X₃ is CR₃; X₄ is CR₄; and X₅ is CR₅ₐ.

### (4) X₁ is CR₁; X₃ is N; X₄ is CR₄; and X₅ is CR₅ₐ.

### (5) X₁ is CR₁; X₃ is CR₃; X₄ is N; and X₅ is CR₅ₐ.

### (6) X₁ is CR₁; X₃ is CR₃; X₄ is CR₄; and X₅ is N.

### (7) X₁ is N; X₃ is N; X₄ is CR₄; and X₅ is CR₅ₐ.

### (8) X₁ is N; X₃ is CR₃; X₄ is N; and X₅ is CR₅ₐ.

### (9) X₁ is N; X₃ is CR₃; X₄ is CR₄; and X₅ₐ is N.

### (10) X₁ is CR₁; X₃ is N; X₄ is N; and X₅ is CR₅ₐ.

### (11) X₁ is CR₁; X₃ is N; X₄ is CR₄; and X₅ is N.

### (12) X₁ is CR₁; X₃ is CR₃; X₄ is N; and X₅ is N.

(13) R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl.
(14) R₁ is hydrogen, cyano, halogen, (C₁₋₄)alkyl, halogen-(C₁₋₄)alkyl, (C₁₋₄)alkoxy, or halogen-(C₁₋₄)alkoxy.
(15) R₁ is hydrogen.
(16) R_{2b} is an aryl or heteroaryl group G₁, which group G₁ is optionally substituted by 1, 2, 3 or 4 substituents independently selected from the group, consisting of cyano, amino, amino-(C₁₋₆)alkyl, (C₁₋₆)alkyl-amino-(C₁₋₆)alkyl, di(C₁₋₄)alkyl-amino-(C₁₋₆)alkyl, aminocarbonyl, thiocarbamoyl, halogen, (C₁₋₆)alkyl, halogen-(C₁₋₆)alkyl, hydroxy, oxo, (C₁₋₆)alkoxy, halogen-(C₁₋₆)alkoxy, (C₁₋₆)alkylthio, halogen-(C₁₋₆)alkylthio, (C₁₋₆)alkoxy-(C₁₋₆)alkyl, (C₃₋₆)cycloalkyl-(C₁₋₆)alkoxy, (C₁₋₆)alkoxy-(C₁₋₆)alkoxy, (C₁₋₆)alkoxy-(C₁₋₆)alkylthio, (C₁₋₆)alkylthio-(C₁₋₆)alkyl, (C₁₋₆)alkylthio-(C₁₋₆)alkoxy, (C₁₋₆)alkylthio-(C₁₋₆)alkylthio, (C₂₋₆)alkenyl, (C₂₋₆)alkynyl, (C₂₋₆)alkenoxy, (C₂₋₆)alkynoxy and a (C₃₋₆)cycloalkyl, aryl, heteroaryl or non-aromatic heterocyclyl group G₂, which group G₂ is optionally substituted by 1 to 4 substituents independently selected from the group, consisting of cyano, aminocarbonyl, halogen, (C₁₋₆)alkyl, halogen-(C₁₋₆)alkyl, hydroxy, (C₁₋₆)alkoxy, halogen-(C₁₋₆)alkoxy, (C₁₋₆)alkylthio, halogen-(C₁₋₆)alkylthio, (C₁₋₆)alkoxy-(C₁₋₆)alkyl, (C₁₋₆)alkoxy-(C₁₋₆)alkoxy, (C₁₋₆)alkoxy-(C₁₋₆)alkylthio, (C₁₋₆)alkylthio-(C₁₋₆)alkyl, (C₁₋₆)alkylthio-(C₁₋₆)alkoxy, (C₁₋₆)alkylthio-(C₁₋₆)alkylthio, (C₂₋₆)alkenyl and (C₂₋₆)alkynyl.
(17) R_{2b} is a heteroaryl group, which is optionally substituted by 1, 2, 3 or 4 substituents independently selected from the group, consisting of cyano, amino, amino-(C₁₋₆)alkyl, (C₁₋₆)alkyl-amino-(C₁₋₆)alkyl, aminocarbonyl, di(C₁₋₄)alkyl-amino-(C₁₋₆)alkyl, aminocarbonyl, thiocarbamoyl, halogen, (C₁₋₆)alkyl, halogen-(C₁₋₆)alkyl, hydroxy, oxo, (C₁₋₆)alkoxy, halogen-(C₁₋₆)alkoxy, (C₁₋₆)alkylthio, halogen-(C₁₋₆)alkylthio, (C₁₋₆)alkoxy-(C₁₋₆)alkyl, (C₃₋₆)cycloalkyl-(C₁₋₆)alkoxy, (C₁₋₆)alkoxy-(C₁₋₆)alkoxy, (C₁₋₆)alkoxy-(C₁₋₆)alkylthio, (C₁₋₆)alkylthio-(C₁₋₆)alkyl, (C₁₋₆)alkylthio-(C₁₋₆)alkoxy, (C₁₋₆)alkylthio-(C₁₋₆)alkylthio, (C₂₋₆)alkenyl, (C₂₋₆)alkynyl, (C₂₋₆)alkenoxy, (C₂₋₆)alkynoxy.
(18) R_{2b} is a 5- or 6-membered heteroaryl group in which structure 1, 2, 3, or 4 ring members are hetero ring members independently selected from the group consisting of a nitrogen ring member, an oxygen ring member and a sulfur ring member, which group is optionally substituted by 1, 2, 3 or 4 substituents independently selected from the group, consisting of cyano, amino, aminocarbonyl, thiocarbamoyl, halogen, (C₁₋₄)alkyl, halogen-(C₁₋₄)alkyl, hydroxy, oxo, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₃₋₄)cycloalkyl-(C₁₋₄)alkoxy, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, (C₂₋₄)alkynyl, (C₂₋₄)alkenoxy, and (C₂₋₄)alkynoxy.
(19) R_{2b} is a 6-membered heteroaryl group in which structure 1, 2, 3, or 4 ring members are hetero ring members independently selected from the group consisting of a nitrogen ring member, an oxygen ring member and a sulfur ring member, which group is optionally substituted by 1, 2, 3 or 4 substituents independently selected from the group, consisting of cyano, amino, aminocarbonyl, thiocarbamoyl, halogen, (C₁₋₄)alkyl, halogen-(C₁₋₄)alkyl, hydroxy, oxo, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₃₋₄)cycloalkyl-(C₁₋₄)alkoxy, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, (C₂₋₄)alkynyl, (C₂₋₄)alkenoxy, and (C₂₋₄)alkynoxy.
(20) R_{2b} is a 6-membered heteroaryl group in which structure 1, 2, 3, or 4 ring members are hetero ring members independently selected from the group consisting of a nitrogen ring member, an oxygen ring member and a sulfur ring member, which group is optionally substituted by 1, 2, 3 or 4 substituents independently selected from the group, consisting of cyano, amino, halogen, (C₁₋₄)alkyl, halogen-(C₁₋₄)alkyl, hydroxy, oxo, (C₁₋₄)alkoxy and halogen-(C₁₋₄)alkoxy.
(21) R_{2b} is a pyridyl or pyrazinyl group which is optionally substituted by 1, 2 or 3 substituents independently selected from the group, consisting of cyano, amino, aminocarbonyl, thiocarbamoyl, halogen, (C₁₋₄)alkyl, halogen-(C₁₋₄)alkyl, hydroxy, oxo, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₃₋₄)cycloalkyl-(C₁₋₄)alkoxy, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, (C₂₋₄)alkynyl, (C₂₋₄)alkenoxy, and (C₂₋₄)alkynoxy.
(22) R_{2b} is a pyridyl or pyrazinyl group which is optionally substituted by 1, 2 or 3 substituents independently selected from the group, consisting of cyano, amino, halogen, (C₁₋₄)alkyl, halogen-(C₁₋₄)alkyl, hydroxy, oxo, (C₁₋₄)alkoxy and halogen-(C₁₋₄)alkoxy.
(23) R_{2b} is a pyridin-2-yl or pyrazin-2-yl group which is optionally substituted by 1, 2 or 3 substituents independently selected from the group, consisting of cyano, amino, aminocarbonyl, thiocarbamoyl, halogen, (C₁₋₄)alkyl, halogen-(C₁₋₄)alkyl, hydroxy, oxo, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₃₋₄)cycloalkyl-(C₁₋₄)alkoxy, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, (C₂₋₄)alkynyl, (C₂₋₄)alkenoxy, and (C₂₋₄)alkynoxy.
(24) R_{2b} is a pyridin-2-yl or pyrazin-2-yl group which is optionally substituted by 1, 2 or 3 substituents independently selected from the group, consisting of cyano, amino, halogen, (C₁₋₄)alkyl, halogen-(C₁₋₄)alkyl, hydroxy, oxo, (C₁₋₄)alkoxy and halogen-(C₁₋₄)alkoxy.
(25) R_{2b} is a pyridin-2-yl or pyrazin-2-yl group which is optionally substituted by 1 or 2 substituents independently selected from the group, consisting of cyano, amino, fluoro, bromo, chloro, hydroxyl, oxo, methyl, fluoromethyl, difluoromethyl, trifluoromethyl, methoxy, fluoromethoxy, difluoromethoxy and trifluoromethoxy.
(26) R_{2b} is a pyridyl or pyrazinyl group which is substituted by 1, 2 or 3 substituents and wherein one of the substituents is located at the para position of the pyridyl or pyrazinyl group relative to the amide linker and wherein the substituents are independently selected from the group, consisting of cyano, amino, aminocarbonyl, thiocarbamoyl, halogen, (C₁₋₄)alkyl, halogen-(C₁₋₄)alkyl, hydroxy, oxo, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₃₋₄)cycloalkyl-(C₁₋₄)alkoxy, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, (C₂₋₄)alkynyl, (C₂₋₄)alkenoxy, and (C₂₋₄)alkynoxy.
(27) R_{2b} is a pyridyl or pyrazinyl group which is substituted by 1, 2 or 3 substituents and wherein one of the substituents is located at the para position of the pyridyl or pyrazinyl group relative to the amide linker and wherein the substituents are independently selected from the group, consisting of cyano, amino, halogen, (C₁₋₄)alkyl, halogen-(C₁₋₄)alkyl, hydroxy, oxo, (C₁₋₄)alkoxy and halogen-(C₁₋₄)alkoxy.
(28) R_{2b} is a pyridin-2-yl or pyrazin-2-yl group which is substituted by 1, 2 or 3 substituents and wherein one of the substituents is located at the para position of the pyridin-2-yl or pyrazin-2-yl group relative to the amide linker and wherein the substituents are independently selected from the group, consisting of cyano, amino, halogen, (C₁₋₄)alkyl, halogen-(C₁₋₄)alkyl, hydroxy, oxo, (C₁₋₄)alkoxy and halogen-(C₁₋₄)alkoxy.
(29) R_{2b} is a pyridyl or pyrazinyl group which is substituted by 2 or 3 substituents and wherein one of the substituents is located at the para position and one of the substituents is located at the ortho position of the pyridyl or pyrazinyl group relative to the amide linker and wherein the substituents are independently selected from the group, consisting of cyano, amino, aminocarbonyl, thiocarbamoyl, halogen, (C₁₋₄)alkyl, halogen-(C₁₋₄)alkyl, hydroxy, oxo, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₃₋₄)cycloalkyl-(C₁₋₄)alkoxy, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, (C₂₋₄)alkynyl, (C₂₋₄)alkenoxy, and (C₂₋₄)alkynoxy.
(30) R_{2b} is a pyridyl or pyrazinyl group which is substituted by 2 or 3 substituents and wherein one of the substituents is located at the para position and one of the substituents is located at the ortho position of the pyridyl or pyrazinyl group relative to the amide linker and wherein the substituents are independently selected from the group, consisting of cyano, amino, halogen, (C₁₋₄)alkyl, halogen-(C₁₋₄)alkyl, hydroxy, oxo, (C₁₋₄)alkoxy and halogen-(C₁₋₄)alkoxy.
(31) R_{2b} is a pyridin-2-yl or pyrazin-2-yl group which is substituted by 2 substituents and wherein one of the substituents is located at the para position and one of the substituents is located at the ortho position of the pyridin-2-yl or pyrazin-2-yl group relative to the amide linker and wherein the substituents are independently selected from the group, consisting of cyano, amino, halogen, (C₁₋₄)alkyl, halogen-(C₁₋₄)alkyl, hydroxy, oxo, (C₁₋₄)alkoxy and halogen-(C₁₋₄)alkoxy.
(32) R_{2b} is a pyridin-2-yl or pyrazin-2-yl group which is substituted by 2 substituents and wherein one of the substituents is located at the para position and one of the substituents is located at the ortho position of the pyridin-2-yl or pyrazin-2-yl group relative to the amide linker and wherein the substituents are independently selected from the group, consisting of cyano, amino, fluoro, bromo, chloro, hydroxyl, oxo, methyl, fluoromethyl, difluoromethyl, trifluoromethyl, methoxy, fluoromethoxy, difluoromethoxy and trifluoromethoxy.
(33) R₃ is hydrogen, cyano, halogen, (C₁₋₄)alkyl, halogen-(C₁₋₄)alkyl, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, or (C₂₋₄)alkynyl.
(34) R₃ is hydrogen, cyano, halogen, (C₁₋₄)alkyl, halogen-(C₁₋₄)alkyl, (C₁₋₄)alkoxy, or halogen-(C₁₋₄)alkoxy.
(35) R₃ is hydrogen.
(36) R₄ is hydrogen, cyano, halogen, (C₁₋₄)alkyl, halogen-(C₁₋₄)alkyl, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, or (C₂₋₄)alkynyl.
(37) R₄ is hydrogen, cyano, halogen, (C₁₋₄)alkyl, halogen-(C₁₋₄)alkyl, (C₁₋₄)alkoxy, or halogen-(C₁₋₄)alkoxy.
(38) R₄ is hydrogen or halogen.
(39) R₄ is hydrogen.
(40) R₄ is fluoro.
(41) R₅ₐ is hydrogen, cyano, halogen, (C₁₋₄)alkyl, halogen-(C₁₋₄)alkyl, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, or (C₂₋₄)alkynyl.
(42) R₅ₐ is hydrogen, cyano, halogen, (C₁₋₄)alkyl, halogen-(C₁₋₄)alkyl, (C₁₋₄)alkoxy, or halogen-(C₁₋₄)alkoxy.
(43) R₅ₐ is hydrogen, or halogen.
(44) R₅ₐ is halogen.
(45) R₅ₐ is fluoro;
(46) R₅ₐ is hydrogen.
(47) R₆ₐ is hydrogen, (C₁₋₄)alkyl, halogen-(C₁₋₄)alkyl, hydroxy-(C₁₋₄)alkyl, (C₁₋₄)alkoxy-(C₁₋₄)alkyl, mercapto-(C₁₋₄)alkyl, (C₁₋₄)alkylthio-(C₁₋₄)alkyl, amino-(C₁₋₄)alkyl, (C₁₋₄)alkyl-amino-(C₁₋₄)alkyl, di(C₁₋₄)alkyl-amino-(C₁₋₄)alkyl, (C₂₋₄)alkenyl, or (C₂₋₄)alkynyl.
(48) R₆ₐ is (C₁₋₃)alkyl or halogen-(C₁₋₃)alkyl.
(49) R₆ₐ is (C₁₋₃)alkyl or fluoro-(C₁₋₃)alkyl.
(50) R₆ is methyl, fluoromethyl, difluoromethyl or trifluoromethyl.

### (51) E₁ is —C(R₇)(R₈)—, or —C(R₇)(R₈)—C(R₉)(R₁₀)—.

### (52) E₁ is —C(R₇)(R₈)—.

### (53) E₂ₐ is —C(R₁₁ₐ)(R₁₂ₐ)—, or —C(R₁₁ₐ)(R₁₂ₐ)—C(R₁₃)(R₁₄)—

### (54) E₂ₐ is —C(R₁₁ₐ)(R₁₂ₐ)—

(55) either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—.
(56) either
(57) each of R₇ and R₈ is independently selected from the group, consisting of hydrogen, cyano, halogen, (C₁₋₃)alkyl and halogen-(C₁₋₃)alkyl;
or
R₇ and R₈, taken together, are oxo or —CH₂—CH₂—.
(58) either
each of R₇ and R₈ is hydrogen;
or
R₇ and R₈, taken together, are oxo.
(59) each of R₇ and R₈ is hydrogen.
(60) either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—.
(61) each of R₉ and R₁₀ is hydrogen.
(62) either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CH₂—CH₂—;
(63) each of R₁₁ₐ and R₁₂ₐ is independently selected from the group, consisting of hydrogen, halogen, (C₁₋₈)alkyl and halogen-(C₁₋₈)alkyl;
each of R₁₁ₐ and R₁₂ₐ is independently selected from the group, consisting of hydrogen, (C₁₋₈)alkyl and halogen-(C₁₋₈)alkyl;
(64) either
(65) each of R₁₁ₐ and R₁₂ₐ is independently selected from the group, consisting of hydrogen, cyano, halogen, (C₁₋₃)alkyl and halogen-(C₁₋₃)alkyl;
or
R₁₁ₐ and R₁₂ₐ, taken together, are oxo or —CR₁₅R₁₆—CR₁₇R₁₈—
wherein R₁₆, R₁₇, R₁₈ and R₁₉ are independently selected from hydrogen and fluoro;
(66) either
each of R₁₁ₐ and R₁₂ₐ is independently selected from the group, consisting of hydrogen, (C₁₋₃)alkyl and halogen-(C₁₋₃)alkyl;
or
R₁₁ₐ and R₁₂ₐ, taken together, are oxo;
(67) either
each of R₁₁ₐ and R₁₂ₐ is independently selected from the group, consisting of hydrogen, methyl and ethyl;
or
R₁₁ₐ and R₁₂ₐ, taken together, are oxo;
(68) each of R₁₁ₐ and R₁₂ₐ is independently selected from the group, consisting of hydrogen, (C₁₋₃)alkyl and halogen-(C₁₋₃)alkyl;
(69) R₁₁ₐ is (C₁₋₈)alkyl, and R₁₂ₐ is halogen-(C₁₋₈)alkyl;
(70) R₁₁ₐ is (C₁₋₃)alkyl, and R₁₂ₐ is halogen-(C₁₋₃)alkyl;
(71) each of R₁₁ₐ and R₁₂ₐ is independently selected from the group, consisting of hydrogen, (C₁₋₃)alkyl and fluoro-(C₁₋₃)alkyl;
(72) each of R₁₁ₐ and R₁₂ₐ is independently selected from the group, consisting of hydrogen, methyl, fluoromethyl, difluoromethyl and trifluoromethyl;
(73) R₁₁ₐ and R₁₂ₐ is hydrogen;
(74) R₁₁ₐ and R₁₂ₐ, taken together, are oxo;
(75) either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—;
(76) each of R₁₃ and R₁₄ is hydrogen.

The skilled person would understand that the embodiments (1) to (76) may be used independently, collectively or in any combination or sub-combination to the limit the scope of the invention as described hereinbefore in relation to compounds of the formula (III), (III′) or (III″).

In another embodiment, the invention relates to a method of treating diseases associated with inhibition of BACE-2 activity comprising administering to a subject a therapeutically effective amount of a compound of the formula (IIIa):

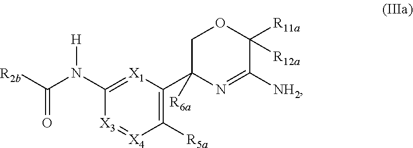

Or a pharmaceutically acceptable salt thereof, wherein:
X₁ is CR₁ or N;
X₃ is CR₃ or N;
X₄ is CR₄ or N;
wherein at least one of X₁, X₃ and X₄ is N and not more than 2 of X₁, X₃ and X₄ are N;
R₁ is hydrogen, cyano, halogen, (C₁₋₄)alkyl, halogen-(C₁₋₄)alkyl, (C₁₋₄)alkoxy, or halogen-(C₁₋₄)alkoxy;
R_{2b} is a 5- or 6-membered heteroaryl group in which structure 1, 2, 3, or 4 ring members are hetero ring members independently selected from the group consisting of a nitrogen ring member, an oxygen ring member and a sulfur ring member, which group is optionally substituted by 1, 2, 3 or 4 substituents independently selected from the group, consisting of cyano, amino, aminocarbonyl, thiocarbamoyl, halogen, (C₁₋₄)alkyl, halogen-(C₁₋₄)alkyl, hydroxy, oxo, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, (C₂₋₄)alkynyl, (C₂₋₄)alkenoxy, and (C₂₋₄)alkynoxy;
R₃, R₄ and R₅ₐ are independently selected from the group consisting of hydrogen, cyano, halogen, (C₁₋₄)alkyl, halogen-(C₁₋₄)alkyl, (C₁₋₄)alkoxy, or halogen-(C₁₋₄)alkoxy;
R₆ₐ is (C₁₋₃)alkyl or fluoro-(C₁₋₃)alkyl; and
each of R₁₁ₐ and R₁₂ₐ is independently selected from the group, consisting of hydrogen, (C₁₋₃)alkyl and halogen-(C₁₋₃)alkyl;
   or a pharmaceutically acceptable salt thereof.

In another embodiment, the invention relates to a method of treating diseases associated with inhibition of BACE-2 activity comprising administering to a subject a therapeutically effective amount of a compound of the formula (IIIa), or a pharmaceutically acceptable salt thereof, wherein:
X₁ is CH or N;
X₃ is CH or N;
X₄ is CR₄ or N;
   wherein one and not more than one of X₁, X₃ and X₄ is N;
R_{2b} is a pyridyl or pyrazinyl group which is optionally substituted by 1, 2 or 3 substituents independently selected from the group, consisting of cyano, amino, aminocarbonyl, thiocarbamoyl, halogen, (C₁₋₄)alkyl, halogen-(C₁₋₄)alkyl, hydroxy, oxo, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, (C₂₋₄)alkynyl, (C₂₋₄)alkenoxy, and (C₂₋₄)alkynoxy;
R₄ and R₅ₐ are independently hydrogen, or halogen;
R₆ₐ is (C₁₋₃)alkyl or fluoro-(C₁₋₃)alkyl; and
each of R₁₁ₐ and R₁₂ₐ is independently selected from the group, consisting of hydrogen, (C₁₋₃)alkyl and fluoro-(C₁₋₃)alkyl;
   or a pharmaceutically acceptable salt thereof.

In another embodiment, the invention relates to a method of treating diseases associated with inhibition of BACE-2 activity comprising administering to a subject a therapeutically effective amount of a compound of the formula (IIIa′):

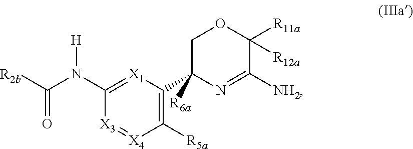

or a pharmaceutically acceptable salt thereof, wherein:
X₁ is CH or N;
X₃ is CH or N;
X₄ is CR₄ or N;
   wherein one and not more than one of X₁, X₃ and X₄ is N;
R_{2b} is a pyridyl or pyrazinyl group which is substituted by 2 or 3 substituents and wherein one of the substituents is located at the para position and one of the substituents is located at the ortho position of the pyridyl or pyrazinyl group relative to the amide linker and wherein the substituents are independently selected from the group, consisting of cyano, amino, halogen, (C₁₋₄)alkyl, halogen-(C₁₋₄)alkyl, hydroxy, oxo, (C₁₋₄)alkoxy and halogen-(C₁₋₄)alkoxy;
R₄ and R₅ₐ are independently hydrogen, or halogen;
R₆ₐ is methyl, fluoromethyl, difluoromethyl or trifluoromethyl; and
each of R₁₁ₐ and R₁₂ₐ is independently selected from the group, consisting of hydrogen, methyl, fluoromethyl, difluoromethyl and trifluoromethyl;
   or a pharmaceutically acceptable salt thereof.

In another embodiment, the invention relates to a method of treating diseases associated with inhibition of BACE-2 activity comprising administering to a subject a therapeutically effective amount of a compound which is selected from:
5-Bromo-pyridine-2-carboxylic acid [6-(5-amino-3-methyl-3,6-dihydro-2H-[1,4]-oxazin-3-yl)-pyridin-2-yl]-amide;
5-Chloro-pyridine-2-carboxylic acid [6-(5-amino-3-fluoromethyl-3,6-dihydro-2H-[1,4]oxazin-3-yl)-pyridin-2-yl]-amide;
5-Bromo-pyridine-2-carboxylic acid [6-(5-amino-3-fluoromethyl-3,6-dihydro-2H-[1,4]oxazin-3-yl)-pyridin-2-yl]-amide;
5-Cyano-3-methyl-pyridine-2-carboxylic acid [6-(5-amino-3-fluoromethyl-3,6-dihydro-2H-[1,4]oxazin-3-yl)-pyridin-2-yl]-amide;
4,6-Dideutero-5-chloro-3-trideuteromethyl-pyridine-2-carboxylic acid [6-(5-amino-3-fluoromethyl-3,6-dihydro-2H-[1,4]oxazin-3-yl)-pyridin-2-yl]-amide;
5-Thiocarbamoyl-pyridine-2-carboxylic acid [6-(5-amino-3-fluoromethyl-3,6-dihydro-2H-[1,4]oxazin-3-yl)-pyridin-2-yl]-amide;
5-Cyano-3-methyl-pyridine-2-carboxylic acid [6-(5-amino-3,6-dimethyl-6-trifluoro-methyl-3,6-dihydro-2H-[1,4]oxazin-3-yl)-pyridin-2-yl]-amide;
5-Cyano-pyridine-2-carboxylic acid [6-(5-amino-3,6-dimethyl-6-trifluoromethyl-3,6-dihydro-2H-[1,4]oxazin-3-yl)-pyridin-2-yl]-amide;
5-Cyano-3-methyl-pyridine-2-carboxylic acid [6-(5-amino-3,6-dimethyl-6-trifluoromethyl-3,6-dihydro-2H-[1,4]oxazin-3-yl)-5-fluoro-pyridin-2-yl]-amide;
4,6-Dideutero-5-chloro-3-trideuteromethyl-pyridine-2-carboxylic acid [4-(5-amino-3-fluoromethyl-3,6-dihydro-2H-[1,4]oxazin-3-yl)-pyridin-2-yl]-amide;
5-Chloro-pyridine-2-carboxylic acid [4-(5-amino-3-fluoromethyl-3,6-dihydro-2H-[1,4]oxazin-3-yl)-pyridin-2-yl]-amide;
3-Methyl-5-thiocarbamoyl-pyridine-2-carboxylic acid [4-(5-amino-3-fluoromethyl-3,6-dihydro-2H-[1,4]oxazin-3-yl)-pyridin-2-yl]-amide;
5-Cyano-3-methyl-pyridine-2-carboxylic acid [4-(5-amino-3,6-dimethyl-6-trifluoromethyl-3,6-dihydro-2H-[1,4]oxazin-3-yl)-5-fluoro-pyridin-2-yl]-amide; and
5-Bromo-pyridine-2-carboxylic acid [5-(5-amino-3-fluoromethyl-3,6-dihydro-2H-[1,4]oxazin-3-yl)-6-chloro-pyridin-3-yl]-amide,
   or a pharmaceutically acceptable salt thereof.

In another embodiment, the invention relates to a method of treating diseases associated with inhibition of BACE-2 activity comprising administering to a subject a therapeutically effective amount of a compound which is selected from:
5-Bromo-pyridine-2-carboxylic acid [6-((R)-5-amino-3-methyl-3,6-dihydro-2H-[1,4]-oxazin-3-yl)-pyridin-2-yl]-amide;
5-Chloro-pyridine-2-carboxylic acid [6-(5-amino-3-fluoromethyl-3,6-dihydro-2H-[1,4]oxazin-3-yl)-pyridin-2-yl]-amide;
5-Bromo-pyridine-2-carboxylic acid [6-(5-amino-3-fluoromethyl-3,6-dihydro-2H-[1,4]oxazin-3-yl)-pyridin-2-yl]-amide;
5-Cyano-3-methyl-pyridine-2-carboxylic acid [6-(5-amino-3-fluoromethyl-3,6-dihydro-2H-[1,4]oxazin-3-yl)-pyridin-2-yl]-amide;
4,6-Dideutero-5-chloro-3-trideuteromethyl-pyridine-2-carboxylic acid [6-(5-amino-3-fluoromethyl-3,6-dihydro-2H-[1,4]oxazin-3-yl)-pyridin-2-yl]-amide;
5-Thiocarbamoyl-pyridine-2-carboxylic acid [6-(5-amino-3-fluoromethyl-3,6-dihydro-2H-[1,4]oxazin-3-yl)-pyridin-2-yl]-amide;
5-Cyano-3-methyl-pyridine-2-carboxylic acid [6-((3R,6R)-5-amino-3,6-dimethyl-6-trifluoro-methyl-3,6-dihydro-2H-[1,4]oxazin-3-yl)-pyridin-2-yl]-amide;
5-Cyano-pyridine-2-carboxylic acid [6-((3S,6R)-5-amino-3,6-dimethyl-6-trifluoromethyl-3,6-dihydro-2H-[1,4]oxazin-3-yl)-pyridin-2-yl]-amide;
5-Cyano-pyridine-2-carboxylic acid [6-((3R,6R)-5-amino-3,6-dimethyl-6-trifluoromethyl-3,6-dihydro-2H-[1,4]oxazin-3-yl)-pyridin-2-yl]-amide;
5-Cyano-3-methyl-pyridine-2-carboxylic acid [6-((3R,6R)-5-amino-3,6-dimethyl-6-trifluoro-methyl-3,6-dihydro-2H-[1,4]oxazin-3-yl)-5-fluoro-pyridin-2-yl]-amide;
5-Cyano-3-methyl-pyridine-2-carboxylic acid [6-((3S,6R)-5-amino-3,6-dimethyl-6-trifluoromethyl-3,6-dihydro-2H-[1,4]oxazin-3-yl)-5-fluoro-pyridin-2-yl]-amide;
4,6-Dideutero-5-chloro-3-trideuteromethyl-pyridine-2-carboxylic acid [4-(5-amino-3-fluoromethyl-3,6-dihydro-2H-[1,4]oxazin-3-yl)-pyridin-2-yl]-amide;
5-Chloro-pyridine-2-carboxylic acid [4-(5-amino-3-fluoromethyl-3,6-dihydro-2H-[1,4]oxazin-3-yl)-pyridin-2-yl]-amide;
3-Methyl-5-thiocarbamoyl-pyridine-2-carboxylic acid [4-(5-amino-3-fluoromethyl-3,6-dihydro-2H-[1,4]oxazin-3-yl)-pyridin-2-yl]-amide;
5-Cyano-3-methyl-pyridine-2-carboxylic acid [4-((3R,6R)-5-amino-3,6-dimethyl-6-trifluoromethyl-3,6-dihydro-2H-[1,4]oxazin-3-yl)-5-fluoro-pyridin-2-yl]-amide;
5-Cyano-3-methyl-pyridine-2-carboxylic acid [4-((3S,6R)-5-amino-3,6-dimethyl-6-trifluoromethyl-3,6-dihydro-2H-[1,4]oxazin-3-yl)-5-fluoro-pyridin-2-yl]-amide; and
5-Bromo-pyridine-2-carboxylic acid [5-(5-amino-3-fluoromethyl-3,6-dihydro-2H-[1,4]oxazin-3-yl)-6-chloro-pyridin-3-yl]-amide,
   or a pharmaceutically acceptable salt thereof.

In one embodiment, the invention relates to a method of treating diseases associated with inhibition of BACE-2 activity comprising administering to a subject a therapeutically effective amount of 5-cyano-pyridine-2-carboxylic acid [3-(5-amino-3-difluoromethyl-3,6-dihydro-2H-[1,4]oxazin-3-yl)-4-fluoro-phenyl]-amide, or a pharmaceutically acceptable salt thereof. In another embodiment, the invention relates to a method of treating diseases associated with inhibition of BACE-2 activity comprising administering to a subject a therapeutically effective amount of 5-cyano-pyridine-2-carboxylic acid [3-((R)-5-amino-3-difluoromethyl-3,6-dihydro-2H-[1,4]oxazin-3-yl)-4-fluoro-phenyl]-amide, or a pharmaceutically acceptable salt thereof. In yet another embodiment, the invention relates to a method of treating diseases associated with inhibition of BACE-2 activity comprising administering to a subject a therapeutically effective amount of 5-cyano-pyridine-2-carboxylic acid [3-((S)-5-amino-3-difluoromethyl-3,6-dihydro-2H-[1,4]oxazin-3-yl)-4-fluoro-phenyl]-amide, or a pharmaceutically acceptable salt thereof.

In a more focused aspect, the invention relates to a method of treating diseases associated with inhibition of BACE-2 activity comprising administering to a subject a therapeutically effective amount of a crystalline form of 5-cyano-pyridine-2-carboxylic acid [3-((R)-5-amino-3-difluoromethyl-3,6-dihydro-2H-[1,4]oxazin-3-yl)-4-fluoro-phenyl]-amide, or a pharmaceutically acceptable salt thereof. In another embodiment, the invention relates to a method of treating diseases associated with inhibition of BACE-2 activity comprising administering to a subject a therapeutically effective amount of a crystalline form of 5-cyano-pyridine-2-carboxylic acid [3-((R)-5-amino-3-difluoromethyl-3,6-dihydro-2H-[1,4]oxazin-3-yl)-4-fluoro-phenyl]-amide which has an X-ray powder diffraction pattern with at least one, two or three peaks having angle of refraction 2 theta (θ) values selected from 8.3, 10.8, 16.6, 18.9, 21.5, 22.2, 23.3, 25.4 and 28.5 when measured using CuK_{α}, radiation, more particularly wherein said values may be plus or minus 0.2° 2θ. In a further embodiment, the invention relates to a method of treating diseases associated with inhibition of BACE-2 activity comprising administering to a subject a therapeutically effective amount of a crystalline form of 5-cyano-pyridine-2-carboxylic acid [3-((R)-5-amino-3-difluoromethyl-3,6-dihydro-2H-[1,4]oxazin-3-yl)-4-fluoro-phenyl]-amide which has an X-ray powder diffraction pattern substantially the same as the X-ray powder diffraction pattern shown in FIG. 1 when measured using CuK_{α}, radiation. For details see Example 152.

The compounds used in the method of the invention may be prepared using the methods disclosed in the Examples herein and in PCT/EP2010/060718, filed on Jul. 23, 2010 and in PCT/EP2010/070502, filed on Dec. 22, 2010. In general, the compounds are prepared by reaction of a compound of formula (IV)

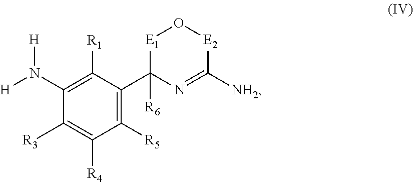

or formula (IVa):

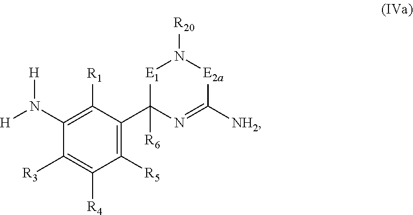

Or formula (IVb):

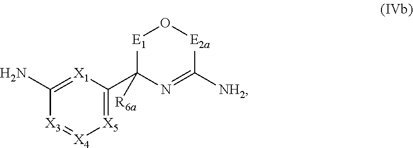

wherein X₁, X₃, X₄, X₅, R₁, R₃, R₄, R₅, R₆, R₆ₐ, R₂₀, E₁, E₂ and E₂ₐ are as defined for formula (I), (II), and (III), in free form or in salt form, with a compound of formula (V):

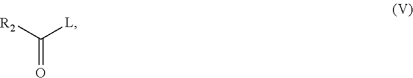

or formula (IVa):

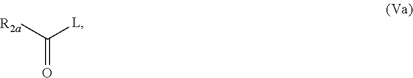

or formula (Vb):

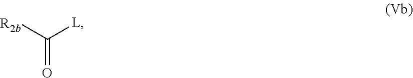

wherein R₂, R₂ₐ and R_{2b} are as defined for formula (I), (II) and (III) and Lisa leaving group, in free form or in salt form,
b) the optional reduction, oxidation or other functionalisation of the resulting compound,
c) the cleavage of any protecting group(s) optionally present and
d) the recovery of the so obtainable compound of the formula (I), (II) or (III) in free form or in salt form.

The working-up of the reaction mixtures and the purification of the compounds thus obtainable may be carried out in accordance with known procedures.

Salts may be prepared from free compounds in known manner, and vice-versa.

Compounds of the invention can also be prepared by further conventional processes, which processes are as described in the Examples.

The starting materials of formulae (IV), (IVa), (IVb), (V), (Va) and (Vb) are known, may be prepared according to conventional procedures starting from known compounds, may be prepared from known compounds as described in the Examples or may be prepared using procedures analogous to those described in the Examples.

Compounds of the invention, in free form or in pharmaceutically acceptable salt form, inhibit BACE-2, and therefore are useful in medicaments for the treatment of type 2 diabetes and other metabolic disorders related to decreased β cell mass and/or function.

The inhibiting properties of a compound of the invention towards proteases can be evaluated in the following test.

### Inhibition of Human BACE-2

Recombinant BACE-2 (extracellular domain, expressed in baculovirus and purified using standard methods) at 0.1 to 10 nM concentrations is incubated with a test compound at various concentrations for 1 hour at room temperature in 10 to 100 mM acetate buffer, pH 4.5, containing 0.1% CHAPS. Synthetic peptide substrate, derived from the sequence of APP and containing a suitable fluorophore-quencher pair, is added to a final concentration of 1 to 5 μM, and the increase in fluorescence is recorded at a suitable excitation/emission wavelength in a microplate spectro-fluorimeter for 5 to 30 minutes in 1-minute intervals. IC₅₀ values are calculated from percentage of inhibition of BACE-2 activity as a function of the test compound concentration.

Compounds of the invention were tested in the above assay. Specific activities of compounds of the invention are described in Example 275.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” of a compound of the invention refers to an amount of the compound of the invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the invention that, when administered to a subject, is effective to (1) at least partially alleviating, inhibiting, preventing and/or ameliorating a condition, or a disorder or a disease (i) mediated by BACE-2 or (ii) associated with BACE-2 activity, or (iii) characterized by activity (normal or abnormal) of BACE-2; or (2) reducing or inhibiting the activity of BACE-2. In another non-limiting embodiment, the term “a therapeutically effective amount” refers to the amount of the compound of the invention that, when administered to a cell, or a tissue, or a non-cellular biological material, or a medium, is effective to at least partially reduce or inhibit the activity of BACE-2.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context. The use of any and all examples, or exemplary language (e.g. “such as”) provided herein is intended merely to better illuminate the invention and does not pose a limitation on the scope of the invention otherwise claimed.

Due to their inhibiting properties towards BACE-2, compounds of the invention may be useful in the treatment or prevention a disease or disorder mediated by BACE-2. Diseases and disorders associated with BACE-2 include: metabolic syndrome (such as dyslipidemia, obesity, insulin resistance, hypertension, microalbuminemia, hyperuricaemia, and hypercoagulability), insulin resistance, glucose intolerance (also known as impaired glucose tolerance or impaired glucose tolerance, IGT), obesity, hypertension, or diabetic complications (such as retinopathy, nephropathy, diabetic foot, ulcers, macroangiopathies, metabolic acidosis or ketosis, reactive hypoglycaemia, hyperinsulinaemia), glucose metabolic disorder, dyslipidaemias of different origins, atherosclerosis and related diseases, high blood pressure, chronic heart failure, Syndrome X, diabetes, non-insulin-dependent diabetes mellitus, Type 2 diabetes, Type 1 diabetes, body weight disorders, weight loss, body mass index and leptin related diseases. In one embodiment, the diseases and conditions include insulin resistance, glucose intolerance, type 2 diabetes, obesity, hypertension, or diabetic complications. In another embodiment, the disease or disorder is impaired glucose tolerance or Type 2 diabetes.

Compounds of the invention are suitable for preventing beta-cell degeneration such as apoptosis or necrosis of pancreatic beta cells, for improving or restoring the functionality of pancreatic cells, and/or increasing the number and/or size of pancreatic beta cells.

As used herein a patient is suffering from “obesity” if the patient exhibits at least one of:
a body mass index (BMI), i.e. the patient's mass (in kg) divided by the square of the patient's height (in m), of 30 or more;
an absolute waist circumference of >102 cm in men or >88 cm in women;
a waist-to-hip ratio >0.9 in men or >0.85 in women; or
a percent body fat >25% in men or >30% in women.

As used herein a patient is suffering from “Type 2 diabetes” if they meet the World Health organisation criteria for Diabetes diagnosis (Definition and diagnosis of diabetes mellitus and intermediate hyperglycaemia, WHO, 2006), i.e. the patient exhibits at least one of:
a fasting plasma glucose ≧7.0 mmol/l (126 mg/dl); or
a venous plasma glucose ≧11.1 mmol/l (200 mg/dl) 2 hours after ingestion of 75 g oral glucose load.

As used herein a patient is suffering from “IGT” if they meet the World Health organisation criteria for IGT diagnosis (Definition and diagnosis of diabetes mellitus and intermediate hyperglycaemia, WHO, 2006), i.e. the patient exhibits both of:
a fasting plasma glucose <7.0 mmol/l (126 mg/dl); and
a venous plasma glucose ≦7.8 and <11.1 mmol/l (200 mg/dl) 2 hours after ingestion of 75 g oral glucose load.

For the above-mentioned indications, the appropriate dosage will vary depending on, for example, the compound employed as active pharmaceutical ingredient, the host, the mode of administration, the nature and severity of the condition, disease or disorder or the effect desired. However, in general, satisfactory results in animals may be obtained at a daily dosage of from about 0.1 to about 100, preferably from about 1 to about 50, mg/kg of animal body weight. In larger mammals, for example humans, an indicated daily dosage is in the range of from about 0.5 to about 2000, preferably from about 2 to about 200, mg of an agent of the invention conveniently administered, for example, in divided doses up to four times a day or in sustained release form.

An agent of the invention may be administered by any conventional route, in particular enterally, preferably orally, e.g. in the form of a tablet or capsule, or parenterally, e.g. in the form of an injectable solution or suspension.

In a further aspect, the invention relates to a pharmaceutical composition comprising an agent of the invention as active pharmaceutical ingredient in association with at least one pharmaceutically acceptable carrier or diluent and optionally in association with other auxiliary substances, such as inhibitors of cytochrome P450 enzymes, agents preventing the degradation of active pharmaceutical ingredients by cytochrome P450, agents improving or enhancing the pharmacokinetics of active pharmaceutical ingredients, agents improving or enhancing the bioavailability of active pharmaceutical ingredients, and so on, e.g. grapefruit juice, ketoconazole or, preferably, ritonavir. Such a composition may be manufactured in conventional manner, e.g. by mixing its components. Unit dosage forms contain, e.g., from about 0.1 to about 1000, preferably from about 1 to about 500, mg of an agent of the invention.

For example, for preclinical animal studies a compound of the invention, such as 5-cyano-pyridine-2-carboxylic acid [3-((R)-5-amino-3-difluoromethyl-3,6-dihydro-2H-[1,4]oxazin-3-yl)-4-fluoro-phenyl]-amide, could be formulated as a suspension in a 0.5% methylcellulose solution with 0.1% Tween80.

In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The present invention further provides anhydrous pharmaceutical compositions and dosage forms comprising the compounds of the present invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

In accordance with the foregoing, in a further aspect, the invention relates to an agent of the invention for use as a medicament, e.g. for the treatment or prevention of a disease or disorder mediated by BACE-2. In a further embodiment, the invention relates to a compound of the invention for use in the treatment of insulin resistance, glucose intolerance, type 2 diabetes, obesity, hypertension, or diabetic complications. In one embodiment, the invention relates to a compound of the invention for use in the treatment of impaired glucose tolerance or Type 2 diabetes.

In a further aspect, the invention relates to the use of a compound of the invention as an active pharmaceutical ingredient in a medicament, e.g. for the treatment or prevention of a disease or disorder mediated by BACE-2. In a further embodiment, the invention relates to the use of a compound of the invention as an active pharmaceutical ingredient in a medicament for the treatment or prevention of insulin resistance, glucose intolerance, type 2 diabetes, obesity, hypertension, or diabetic complications. In one embodiment, the invention relates to the use of a compound of the invention as an active pharmaceutical ingredient in a medicament for the treatment or prevention of impaired glucose tolerance or Type 2 diabetes.

In a further aspect, the invention relates to the use of an agent of the invention for the manufacture of a medicament for the treatment or prevention of a disease or disorder mediated by BACE-2. In a further embodiment, the invention relates to the use of an agent of the invention for the manufacture of a medicament for the treatment or prevention of insulin resistance, glucose intolerance, type 2 diabetes, obesity, hypertension, or diabetic complications. In one embodiment, the invention relates to the use of an agent of the invention for the manufacture of a medicament for the treatment or prevention of impaired glucose tolerance or Type 2 diabetes.

In a further aspect, the invention relates to a method for the treatment or prevention of a disease or disorder mediated by BACE-2, in a subject in need of such treatment, prevention or suppression, which method comprises administering to such subject a therapeutically effective amount of a compound of the invention. In one embodiment, the invention relates to a method of inhibiting BACE-2 activity in a subject, wherein the method comprises administering to the subject a therapeutically effective amount of a compound of the invention. In another embodiment, the invention relates to a method for the treatment or prevention of insulin resistance, glucose intolerance, type 2 diabetes, obesity, hypertension, or diabetic complications, in a subject in need of such treatment or prevention, which method comprises administering to such subject a therapeutically effective amount of a compound of the invention. In yet another embodiment, the invention relates to a method for the treatment or prevention of impaired glucose tolerance or Type 2 diabetes, in a subject in need of such treatment or prevention, which method comprises administering to such subject a therapeutically effective amount of a compound of the invention.

A compound of the invention can be administered as sole active pharmaceutical ingredient or as a combination with at least one other active pharmaceutical ingredient effective, e.g., in the treatment or prevention of a disease or disorder mediated by BACE-2, such as impaired glucose tolerance or type 2 diabetes. Such a pharmaceutical combination may be in the form of a unit dosage form, which unit dosage form comprises a predetermined quantity of each of the at least two active components in association with at least one pharmaceutically acceptable carrier or diluent. Alternatively, the pharmaceutical combination may be in the form of a package comprising the at least two active components separately, e.g. a pack or dispenser-device adapted for the concomitant or separate administration of the at least two active components, in which these active components are separately arranged. In a further aspect, the invention relates to such pharmaceutical combinations.

In a further aspect, the invention therefore relates to a pharmaceutical combination comprising a therapeutically effective amount of a compound of the invention, or a pharmaceutically acceptable salt thereof, and a second drug substance, for simultaneous or sequential administration.

In one embodiment, the invention provides a product comprising a compound of the invention, or a pharmaceutically acceptable salt thereof, and at least one other therapeutic agent as a combined preparation for simultaneous, separate or sequential use in therapy. In one embodiment, the therapy is the treatment of a disease or condition mediated by BACE-2, such as impaired glucose tolerance or type 2 diabetes.

In one embodiment, the invention provides a pharmaceutical composition comprising a compound of the invention, or a pharmaceutically acceptable salt thereof, and another therapeutic agent(s). Optionally, the pharmaceutical composition may comprise a pharmaceutically acceptable excipient, as described above.

In one embodiment, the invention provides a kit comprising two or more separate pharmaceutical compositions, at least one of which contains a compound of the invention, or a pharmaceutically acceptable salt thereof. In one embodiment, the kit comprises means for separately retaining said compositions, such as a container, divided bottle, or divided foil packet. An example of such a kit is a blister pack, as typically used for the packaging of tablets, capsules and the like. The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the invention, or pharmaceutically acceptable salt thereof, and the other therapeutic agent may be manufactured and/or formulated by the same or different manufacturers. Moreover, the compound of the invention, or pharmaceutically acceptable salt thereof, and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention, or pharmaceutically acceptable salt thereof, and the other therapeutic agent. Accordingly, the invention provides a compound of the invention, or pharmaceutically acceptable salt thereof, for use in the treatment of a disease or condition mediated by BACE-2, such as impaired glucose tolerance or type 2 diabetes, wherein the medicament is prepared for administration with another therapeutic agent. The invention also provides the use of another therapeutic agent for treating a disease or condition mediated by BACE-2, such as impaired glucose tolerance or type 2 diabetes, wherein the medicament is administered with a compound of the invention, or pharmaceutically acceptable salt thereof.

The invention also provides a compound of the invention, or a pharmaceutically acceptable salt thereof, for use in a method of treating a disease or condition mediated by BACE-2, such as impaired glucose tolerance or type 2 diabetes, wherein the compound of the invention, or a pharmaceutically acceptable salt thereof, is prepared for administration with another therapeutic agent. The invention also provides another therapeutic agent for use in a method of treating a disease or condition mediated by BACE-2, such as impaired glucose tolerance or type 2 diabetes, wherein the other therapeutic agent is prepared for administration with a compound of the invention, or a pharmaceutically acceptable salt thereof. The invention also provides a compound of the invention, or a pharmaceutically acceptable salt thereof, for use in a method of treating a disease or condition mediated by BACE-2, such as impaired glucose tolerance or type 2 diabetes, wherein the compound of the invention, or a pharmaceutically acceptable salt thereof, is administered with another therapeutic agent. The invention also provides another therapeutic agent for use in a method of treating a disease or condition mediated by BACE-2, such as impaired glucose tolerance or type 2 diabetes, wherein the other therapeutic agent is administered with a compound of the invention, or a pharmaceutically acceptable salt thereof.

The invention also provides the use of a compound of the invention, or a pharmaceutically acceptable salt thereof, for treating a disease or condition mediated by BACE-2, such as impaired glucose tolerance or type 2 diabetes, wherein the patient has previously (e.g. within 24 hours) been treated with another therapeutic agent. The invention also provides the use of another therapeutic agent for treating a disease or condition mediated by BACE-2, such as impaired glucose tolerance or type 2 diabetes, wherein the patient has previously (e.g. within 24 hours) been treated with a compound of the invention, or a pharmaceutically acceptable salt thereof.

In one embodiment, the invention relates to a compound of the invention, or a pharmaceutically acceptable salt thereof, in combination with another therapeutic agent wherein the other therapeutic agent is selected from:

a) antidiabetic agents, such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; protein tyrosine phosphatase-1B (PTP-1B) inhibitors such as PTP-112; GSK3 (glycogen synthase kinase-3) inhibitors such as SB-517955, SB-4195052, SB-216763, NN-57-05441 and NN-57-05445; RXR ligands such as GW-0791 and AGN-194204; sodium-dependent glucose cotransporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; and DPPIV (dipeptidyl peptidase IV) inhibitors such as vildagliptin;
b) hypolipidemic agents such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; cholestyramine; fibrates; nicotinic acid bile acid binding resins such as cholestyramine; fibrates; nicotinic acid and other GPR109 agonists; cholesterol absorption inhibitors such as ezetimibe; CETP inhibitors (cholesterol-ester-transfer-protein inhibitors), and aspirin;
c) anti-obesity agents such as orlistat, sibutramine and Cannabinoid Receptor 1 (CB1) antagonists e.g. rimonabant; and
d) anti-hypertensive agents, e.g., loop diuretics such as ethacrynic acid, furosemide and torsemide; angiotensin converting enzyme (ACE) inhibitors such as benazepril, captopril, enalapril, fosinopril, lisinopril, moexipril, perinodopril, quinapril, ramipril and trandolapril; inhibitors of the Na-K-ATPase membrane pump such as digoxin; neutralendopeptidase (NEP) inhibitors; ACE/NEP inhibitors such as omapatrilat, sampatrilat and fasidotril; angiotensin II antagonists such as candesartan, eprosartan, irbesartan, losartan, telmisartan and valsartan, in particular valsartan; renin inhibitors such as ditekiren, zankiren, terlakiren, aliskiren, RO 66-1132 and RO-66-1168; β-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; and aldosterone synthase inhibitors.
e) agonists of peroxisome proliferator-activator receptors, such as fenofibrate, pioglitazone, rosiglitazone, tesaglitazar, BMS-298585, L-796449, the compounds specifically described in the patent application WO 2004/103995 i.e. compounds of examples 1 to 35 or compounds specifically listed in claim 21, or the compounds specifically described in the patent application WO 03/043985 i.e. compounds of examples 1 to 7 or compounds specifically listed in claim 19 and especially (R)-1-{4-[5-methyl-2-(4-trifluoromethyl-phenyl)-oxazol-4-ylmethoxy]-benzenesulfonyl}-2,3-dihydro-1H-indole-2-carboxylic or a salt thereof.

Thus the invention covers pharmaceutical compositions comprising;

i) a compound of the invention, or a pharmaceutically acceptable salt thereof, and
ii) at least one compound selected from
a) antidiabetic agents,
b) hypolipidemic agents,
c) anti-obesity agents,
d) anti-hypertensive agents,
e) agonists of peroxisome proliferator-activator receptors, and
   ii) one or more pharmaceutically acceptable carriers.

Other specific anti-diabetic compounds are described by Patel Mona in Expert Opin Investig Drugs, 2003, 12(4), 623-633, in the FIGS. 1 to 7, which are herein incorporated by reference. The structure of the therapeutic agents identified by code numbers, generic or trade names may be taken from the actual edition of the standard compendium “The Merck Index” or from databases, e.g., Patents International (e.g. IMS World Publications). The corresponding content thereof is hereby incorporated by reference.

Accordingly, the present invention provides pharmaceutical compositions comprising a therapeutically effective amount of a compound of the invention, or a pharmaceutically acceptable salt thereof, in combination with a therapeutically effective amount of another therapeutic agent, preferably selected from anti-diabetics, hypolipidemic agents, anti-obesity agents or anti-hypertensive agents, most preferably from antidiabetics or hypolipidemic agents as described above.

The following Examples illustrate the invention, but do not limit it.

### EXAMPLES

### Abbreviations

ACN acetonitrile
AcOH acetic acid
aq. aqueous
Boc tert-butoxycarbonyl
t-Bu tert-butyl
t-BuOH tert-butanol
conc. concentrated
DAST diethylaminosulfurtrifluoride (Et₂N)₂SF₃
DCM dichloromethane
DIAD diisopropyl azodicarboxylate
DIPEA diisopropylethylamine
DMF dimethylformamide
DMSO dimethylsulfoxide
DPPF 1,1′-bis(diphenylphosphino)ferrocene
ee enantiomeric excess
EDC 1-(3-dimethylaminopropyl)-3-ethylcarbodiimide hydrochloride
eq equivalent(s)
ESI electrospray ionisation
Et₃N triethylamine
Et₂O diethylether
EtOAc ethyl acetate
EtOH ethanol
h hour(s)
Hex hexane
HMDS hexamethyldisilazane
HOAt 1-hydroxy-7-aza-benztriazole
HOBT hydroxy-benztriazole
HPLC high performance liquid chromatography
LCMS liquid chromatography with mass spectrometry
LDA lithium diisopropylamide
MeOH methanol
min minute(s)
MS mass spectrometry
NMR nuclear magnetic resonance spectrometry
NP normal phase
PE petrolether
PPh3 triphenylphosphine
Rf retention factor (TLC)
RP reverse phase
Rt retention time
rt room temperature
sat. Saturated
SMB simulated moving bed
Soln. solution
TBME tert-butyl-methyl-ether
TFA trifluoroacetic acid
THF tetrahydrofuran
TLC thin layer chromatography
UPLC ultra performance liquid chromatography

### General Chromatography Information

HPLC method H1 (Rt_{H1}):
HPLC-column dimensions: 3.0×30 mm
HPLC-column type: Zorbax SB-18, 1.8 μm
HPLC-eluent: A) water+0.05 Vol.-% TFA; B) ACN+0.05 Vol.-% TFA
HPLC-gradient: 30-100% B in 3.25 min, flow=0.7 ml/min
   HPLC method H2 (Rt_{H2}):
HPLC-column dimensions: 3.0×30 mm
HPLC-column type: Zorbax SB-C18, 1.8 μm
HPLC-eluent: A) water+0.05 Vol.-% TFA; B) ACN+0.05 Vol.-% TFA
HPLC-gradient: 0-100% B in 3.25 min, flow=0.7 ml/min
   LCMS method H3 (Rt_{H3}):
HPLC-column dimensions: 3.0×30 mm
HPLC-column type: Zorbax SB-C18, 1.8 μm
HPLC-eluent: A) water+0.05 Vol.-% TFA, B) ACN+0.05 Vol.-% TFA
HPLC-gradient: 10-100% B in 3.25 min, flow=0.7 ml/min
   LCMS method H4 (Rt_{H4}):
HPLC-column dimensions: 3.0×30 mm
HPLC-column type: Zorbax SB-C8, 1.8 μm
HPLC-eluent: A) water+0.05 Vol.-% TFA, B) ACN+0.05 Vol.-% TFA
HPLC-gradient: 10-95% B in 2.00 min, 95% B 2.00 min, flow=0.7 ml/min
   UPLC method H5 (Rt_{H5}):
HPLC-column dimensions: 2.1×50 mm
HPLC-column type: Acquity UPLC HSS T3 C18, 1.7 μm
HPLC-eluent: A) water+0.1 Vol.-% TFA, B) ACN+0.1 Vol.-% TFA
HPLC-gradient: 5-100% B in 1.5 min, flow=1.0 ml/min
   LCMS method H6 (Rt_{H6}):
HPLC-column dimensions: 3.0×30 mm
HPLC-column type: Zorbax SB-C18, 1.8 μm
HPLC-eluent: A) water+0.05 Vol.-% TFA; B) ACN+0.05 Vol.-% TFA
HPLC-gradient: 40-100% B in 3.25 min, flow=0.7 ml/min
   LCMS method H7 (Rt_{H7}):
HPLC-column dimensions: 3.0×30 mm
HPLC-column type: Zorbax SB-C18, 1.8 μm
HPLC-eluent: A) water+0.05 Vol.-% TFA; B) ACN+0.05 Vol.-% TFA
HPLC-gradient: 50-100% B in 3.25 min, flow=0.7 ml/min
   LCMS method H8 (Rt_{H8}):
HPLC-column dimensions: 4.0×20 mm
HPLC-column type: Mercury MS Synergi, 2 μm
HPLC-eluent: A) water+0.1 Vol.-% formic acid, B) ACN
HPLC-gradient: 0.5 min 30% B, 30-95% B in 1 min, 0.9 min 95% B, flow=2.0 ml/min
HPLC-column temperature: 30° C.
   LCMS method H9 (Rt_{H9}):
HPLC-column dimensions: 4.0×20 mm
HPLC-column type: Mercury MS Synergi, 2 μm
HPLC-eluent: A) water+0.1 Vol.-% formic acid, B) ACN
HPLC-gradient: 0.5 min 70% B, 70-100% B in 1 min, 0.6 min 70% B, flow=2.0 ml/min
HPLC-column temperature: 30° C.
   UPLC method H10 (Rt_{H10}):
HPLC-column dimensions: 2.1×50 mm
HPLC-column type: Acquity UPLC HSS T3, 1.8 μm
HPLC-eluent: A) water+0.05 Vol.-% formic acid+3.75 mM ammonium acetate B) ACN+0.04 Vol.-% formic acid
HPLC-gradient: 2-98% B in 1.7 min, 98% B 0.45 min, flow=1.2 ml/min
   LCMS method H11 (Rt_{H11}):
HPLC-column dimensions: 2.1×30 mm
HPLC-column type: Ascentis Express C18, 2.8 μm
HPLC-eluent: A) water+0.05 Vol.-% formic acid+3.75 mM ammonium acetate, B) ACN+0.04 Vol.-% formic acid
HPLC-gradient: 2-98% B in 1.4 min, 0.75 min 98% B, flow=1.2 ml/min
HPLC-column temperature: 50° C.
   UPLC method H12 (Rt_{H12}): (SQ22=SQ04 old method)
HPLC-column dimensions: 2.1×50 mm
HPLC-column type: Acquity UPLC HSS T3, 1.8 μm
HPLC-eluent: A) water+0.1 Vol.-% formic acid, B) ACN+0.1% formic acid
HPLC-gradient: 10-95% B in 1.5 min, 1.0 min 95% B, flow=1.2 ml/min
HPLC-column temperature: 50° C.
   UPLC method H13 (Rt_{H13}): (SQ02, SQ12)
HPLC-column dimensions: 2.1×50 mm
HPLC-column type: Acquity UPLC HSS T3, 1.8 μm
HPLC-eluent: A) water+0.05 Vol.-% formic acid+3.75 mM ammonium acetate B) ACN+0.04 Vol.-% formic acid
HPLC-gradient: 2-98% B in 1.4 min, 98% B 0.45 min, flow=1.2 ml/min
HPLC-column temperature: 50° C.
   UPLC method H14 (Rt_{H14}):
HPLC-column dimensions: 2.1×50 mm
HPLC-column type: Acquity UPLC HSS T3, 1.8 μm
HPLC-eluent: A) water+0.05 Vol.-% formic acid+3.75 mM ammonium acetate B) ACN+0.04 Vol.-% formic acid
HPLC-gradient: 2-98% B in 1.4 min, 98% B 0.75 min, flow=1.2 ml/min
HPLC-column temperature: 50° C.
   HPLC method H15 (Rt_{H15}):
HPLC-column dimensions: 4.6×150 mm
HPLC-column type: Zorbax XDB-C18, 5 μm
HPLC-eluent: A) water+0.01 Vol.-% TFA; B) ACN/MeOH 1:1
HPLC-gradient: 1 min 30% B, 30-100% B in 5 min, 100-30% B in 4 min, flow=1.0 ml/min
HPLC-column temperature: 40° C.
   HPLC method 16 (Rt_{H16}):
HPLC-column dimensions: 4.6×150 mm
HPLC-column type: Zorbax XDB-C18, 5 μm
HPLC-eluent: A) water+0.01 Vol.-% TFA; B) ACN/MeOH 1:1
HPLC-gradient: 1 min 5% B, 5-100% B in 5 min, 100-5% B in 4 min, flow=1.0 ml/min
HPLC-column temperature: 40° C.

### Example 1

### Furan-2-carboxylic acid [3-(5-amino-3-methyl-3,6-dihydro-2H-[1,4]oxazin-3-yl)-phenyl]-amide hydrochloride

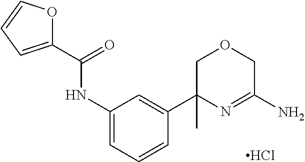 [SMILES: CC1(c2cccc(NC(=O)c3ccco3)c2)COCC(N)=N1 ; Cl]

### a) 2-Amino-2-(3-bromo-phenyl)-propionitrile

A mixture of 1-(3-bromo-phenyl)-ethanone (10 g, 50 mmol), NH₄Cl (6.4 g, 100 mmol) and KCN (6.5 g, 100 mmol) was dissolved in ammonia (200 ml). The solution was stirred at room temperature for 3 days. The mixture was extracted with diethylether (3×300 ml). The organic phase was washed with water and brine, dried with Na₂SO₄ and concentrated in vacuo to yield the title compound (also containing some unreacted starting material). ¹H-NMR (400 MHz, CDCl₃): 7.84 (s, 1H), 7.59 (d, 1H), 7.48 (d, 1H), 7.28 (m, 1H), 1.75 (s, 3H).

### b) 2-Amino-2-(3-bromo-phenyl)-propionic acid hydrochloride

2-Amino-2-(3-bromo-phenyl)-propionitrile (10 g, 44 mmol) was added to concentrated hydrochloric acid (100 ml) at room temperature. The mixture was refluxed overnight and then concentrated in vacuo to give a crude product, which was washed with EtOAc to yield the pure title compound. ¹H-NMR (400 MHz, CD₃OD): 7.62 (m, 2H), 7.48 (m, 2H), 1.82 (s, 3H).

### c) 2-Amino-2-(3-bromo-phenyl)-propan-1-ol

NaBH₄ (38 g, 1.125 mol) was added at room temperature to a slurry of 2-amino-2-(3-bromo-phenyl)-propionic acid hydrochloride (105 g, 375 mmol) in dry THF. At 0° C. BF₃—O(C₂H₅)₂ (158 g, 1.125 mol) was added dropwise. The mixture was allowed to warm to room temperature, stirred for three days, quenched with 1M aqueous NaOH solution, concentrated in vacuo to remove the THF and extracted with EtOAc (3×300 ml). The organic phase was washed with 1M aqueous NaOH solution, dried with sodium sulfate and concentrated in vacuo to yield the title compound, which was used in the next reaction step without further purification. ¹H-NMR (400 MHz, CDCl₃): 7.61 (s, 1H), 7.35 (m, 2H), 7.21 (m, 1H), 3.58 (q, 2H), 1.42 (s, 3H).

### d) N-[1-(3-Bromo-phenyl)-2-hydroxy-1-methyl-ethyl]-2-chloro-acetamide

2-Chloroacetyl chloride (2.24 g, 19.8 mmol) was added dropwise at 0° C. to a suspension of 2-amino-2-(3-bromo-phenyl)-propan-1-ol (3.8 g, 16.5 mmol), K₂CO₃ (4.55 g, 33 mmol) and dichloromethane (40 ml). The mixture was allowed to warm to room temperature over a period of approximately 3 h, washed with 1N hydrochloric acid and brine, dried with Na₂SO₄ and evaporated in vacuo to yield the crude title compound. ¹H-NMR (400 MHz, CDCl₃): 7.43 (m, 2H), 7.23 (m, 2H), 4.10-4.03 (m, 4H), 1.71 (s, 3H).

### e) 5-(3-Bromo-phenyl)-5-methyl-morpholin-3-one

The crude N-[1-(3-bromo-phenyl)-2-hydroxy-1-methyl-ethyl]-2-chloro-acetamide (70 g, 230 mmol) was dissolved in tert-butanol (11). The solution was treated with portions of potassium tert-butoxide (52 g, 460 mmol). The mixture was refluxed for 30 min, after cooling quenched with water and evaporated. The residue was dissolved in EtOAc (500 ml) and washed with water and brine. The organic phase was dried with Na₂SO₄ and concentrated in vacuo to yield the crude title compound. The crude product was purified by chromatography on silica gel (PE/EtOAc=20:1 to 1:1) to give the title compound in the form of a grey solid. ¹H-NMR (400 MHz, DMSO-d₆): 8.66 (s, 1H), 7.60 (s, 1H), 7.48 (d, 1H), 7.44 (d, 1H), 7.34 (t, 1H), 4.02 (s, 2H), 3.92 (d, 1H), 3.68 (d, 1H), 1.38 (s, 3H).

### f) 5-(3-Bromo-phenyl)-5-methyl-morpholine-3-thione

A solution of 5-(3-bromo-phenyl)-5-methyl-morpholin-3-one (18 g, 67 mmol) in dry THF was treated with Lawesson's reagent (27 g, 67 mmol) in one portion at room temperature. The mixture was refluxed for 2 h. The title compound was obtained by chromatography on silica gel (PE/EtOAc=30:1 to 10:1). ¹H-NMR (400 MHz, DMSO-d₆): 11.08 (s, 1H), 7.50 (m, 2H), 7.35 (m, 2H), 4.36 (s, 2H), 4.00 (m, 1H), 3.73 (m, 1H), 1.51 (s, 3H).

### g) 5-(3-Bromo-phenyl)-5-methyl-5,6-dihydro-2H-[1,4]oxazin-3-ylamine

To a solution of 5-(3-bromo-phenyl)-5-methyl-morpholine-3-thione (5 g, 17.5 mmol) in MeOH/NH₃ (110 ml) were added at room temperature t-BuOOH (28 ml, 65%) and NH₄OH (47 ml, 25%). The mixture was stirred overnight, quenched with aqueous Na₂S₂O₃ solution, concentrated in vacuo to remove the methanol solution and extracted with EtOAc (3×30 ml). The organic phase was dried with Na₂SO₄ and concentrated in vacuo to give the crude product, which was purified by preparative HPLC [column: Venusil XBP-C18, 250×21.2 mm, 10 μm; injection volume: 10 ml/injection; mobile phase: CH₃CN/H₂O=10 to 35% (0.1% formic acid) gradient for 15 min, washed with 95% CH₃CN for 4 min, back to 10% balance for 4 min] to give the title compound in the form of a formic acid salt. ¹H-NMR (300 MHz, DMSO-d₆): 9.99 (s, 1H), 8.39 (s, 1H), 7.65 (s, 1H), 7.55 (d, 1H), 7.47 (d, 1H), 7.39 (t, 1H), 4.46 (s, 2H), 4.05 (d, 1H), 3.85 (d, 1H), 1.55 (s, 3H).

### h) [5-(3-Bromo-phenyl)-5-methyl-5,6-dihydro-2H-[1,4]oxazin-3-yl]-carbamic acid tert-butyl ester

A mixture of 5-(3-bromo-phenyl)-5-methyl-5,6-dihydro-2H-[1,4]oxazin-3-ylamine (4.73 g, 15 mmol) and dichloromethane was cooled to 0° C., treated with (Boc)₂O (4.26 g, 19.5 mmol) and DIPEA (2.91 g, 22.5 mmol) and stirred for 17 h at room temperature. 300 ml of water were added dropwise, the phases were separated, the aqueous phase was extracted twice with dichloromethane, and the combined organic phases were washed with 1M aqueous HCl solution and water, dried with Na₂SO₄ and evaporated under reduced pressure to yield the title compound. ¹H-NMR (500 MHz, DMSO-d₆): 9.58 (br, 1H), 7.62 (s, 1H), 7.40-7.25 (m, 3H), 4.50-4.30 (m, 2H), 3.75-3.35 (m, 2H), 1.45 (s, 3H), 1.41 (s, 9H); MS: 369, 371 [(M+H)⁺].

### i) [5-(3-Azido-phenyl)-5-methyl-5,6-dihydro-2H-[1,4]oxazin-3-yl]-carbamic acid tert-butyl ester

[5-(3-Bromo-phenyl)-5-methyl-5,6-dihydro-2H-[1,4]oxazin-3-yl]-carbamic acid tert-butyl ester (5.03 g, 12.67 mmol), sodium azide (1.647 g, 25.3 mmol), sodium ascorbate (0.125 g, 0.63 mmol), copper iodide (0.241 g, 1.27 mmol) and (1R,2R)—N,N′-dimethyl-cyclohexane-1,2-diamine (0.270 g, 1.90 mmol) were dissolved in ethanol (17.7 ml) and water (7.6 ml). The mixture was stirred under N₂ at 90° C. for 4 h and then poured into 1M aqueous KHCO₃ solution. The mixture was extracted with EtOAc, and the organic phase was washed with brine, dried with Na₂SO₄ and evaporated under reduced pressure. The residue was purified by chromatography on silica gel (cyclohexane/EtOAc=7:3) to yield the title compound. ¹H-NMR (500 MHz, DMSO-d₆): 9.57 (br, 1H), 7.38 (m, 1H), 7.24 (d, 1H), 7.18 (br, 1H), 7.0 (br, 1H), 4.50-4.30 (m, 2H), 3.75-3.35 (m, 2H), 1.41 (s, 9H), 1.36 (s, 3H); MS: 332 [(M+H)⁺].

### j) [5-(3-Amino-phenyl)-5-methyl-5,6-dihydro-2H-[1,4]oxazin-3-yl]-carbamic acid tert-butyl ester

A solution of [5-(3-azido-phenyl)-5-methyl-5,6-dihydro-2H-[1,4]oxazin-3-yl]-carbamic acid tert-butyl ester (497 mg, 1.50 mmol) in EtOAc (37 ml) was hydrogenated using Lindlar catalyst (10 h, room temperature). The mixture was filtered through Celite, and the filtrate was evaporated under reduced pressure yielding the title compound in the form of a colourless solid. ¹H-NMR (500 MHz, DMSO-d₆): 9.57 (br, 1H), 6.97 (br, 1H), 6.55 (s, 1H), 6.52 (d, 1H), 6.45 (br, 1H), 5.08 (br, 2H), 4.40-4.30 (m, 2H), 3.75-3.45 (m, 2H), 1.47 (s, 3H), 1.39 (s, 9H); MS: 306 [(M+H)⁺].

### k) (5-{3-[(Furan-2-carbonyl)-amino]-phenyl}-5-methyl-5,6-dihydro-2H-[1,4]oxazin-3-yl)-carbamic acid tert-butyl ester

[5-(3-Amino-phenyl)-5-methyl-5,6-dihydro-2H-[1,4]oxazin-3-yl]-carbamic acid tert-butyl ester (264 mg, 0.865 mmol), furan-2-carboxylic acid (107 mg, 0.951 mmol) and HOBT (172 mg, 1.124 mmol) were dissolved in dichloromethane under N₂ at 0° C. DIPEA (112 mg, 0.865 mmol) and EDC (182 mg, 0.951 mmol) were added. The mixture was stirred at 0° C. for 10 min, then allowed to warm to room temperature, stirred for 17 h at room temperature, quenched with 1M aqueous KHCO₃ solution and extracted with dichloromethane. The organic phase was washed with water and brine, dried with Na₂SO₄ and concentrated under reduced pressure. The residue was purified by chromatography on silica gel (cyclohexane/EtOAc) to yield the title compound in the form of a colourless solid. ¹H-NMR (400 MHz, DMSO-d₆): 9.86 (br, 1H), 9.27 (br, 1H), 7.83 (d, 1H), 7.69 (m, 2H), 7.30 (m, 2H), 7.15 (dd, 1H), 6.65 (m, 1H), 4.40-4.30 (m, 2H), 3.75-3.55 (m, 2H), 1.52 (s, 3H), 1.44 (s, 9H); MS: 400 [(M+H)⁺].

### l) Furan-2-carboxylic acid [3-(5-amino-3-methyl-3,6-dihydro-2H-[1,4]oxazin-3-yl)-phenyl]-amide hydrochloride

A solution of (5-{3-[(furan-2-carbonyl)-amino]-phenyl}-5-methyl-5,6-dihydro-2H-[1,4]oxazin-3-yl)-carbamic acid tert-butyl ester (39.9 mg, 0.1 mmol) in dichloromethane was treated with 4M HCl in dioxane (40 eq). The mixture was warmed to 40° C. for 10 h and then evaporated under reduced pressure to yield the title compound (hydrochloride salt) in the form of a colourless solid. ¹H-NMR (500 MHz, DMSO-d₆): 10.65 (1H, NH⁺), 10.31 (s, 1H), 9.14 (br, 1H), 8.52 (br, 1H), 7.95 (s, 1H), 7.82 (s, 1H), 7.77 (d, 1H), 7.40 (m, 2H), 7.18 (d, 1H), 6.72 (m, 1H), 4.59 (s, 2H), 3.87 (dd, 2H), 1.64 (s, 3H); MS: 300 [(M+H)⁺].

### Example 2

### 5-Bromo-pyridine-2-carboxylic acid [3-(5-amino-3-methyl-3,6-dihydro-2H-[1,4]oxazin-3-yl)-phenyl]-amide hydrochloride

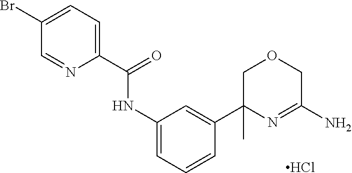 [SMILES: CC1(c2cccc(NC(=O)c3ccc(Br)cn3)c2)COCC(N)=N1 ; Cl]

### a) (5-{3-[(5-Bromo-pyridine-2-carbonyl)-amino]-phenyl}-5-methyl-5,6-dihydro-2H-[1,4]oxazin-3-yl)-carbamic acid tert-butyl ester

[5-(3-Amino-phenyl)-5-methyl-5,6-dihydro-2H-[1,4]oxazin-3-yl]-carbamic acid tert-butyl ester (264 mg, 0.865 mmol), 5-bromo-pyridine-2-carboxylic acid (192 mg, 0.951 mmol) and HOBT (172 mg, 1.124 mmol) were dissolved in dichloromethane under N₂ at 0° C. DIPEA (112 mg, 0.865 mmol) and EDC (182 mg, 0.951 mmol) were added. The mixture was stirred at 0° C. for 10 min, then allowed to warm to room temperature, stirred for 17 h at room temperature, quenched with 1M aqueous KHCO₃ solution and extracted with dichloromethane. The organic phase was washed with water and brine, dried with Na₂SO₄ and concentrated under reduced pressure. The residue was purified by chromatography on silica gel (cyclohexane/EtOAc) to yield the title compound in the form of a colourless solid. ¹H-NMR (400 MHz, DMSO-d₆, 81° C.): 10.32 (1H, NH), 9.30 (br, 1H), 8.81 (s, 1H), 8.29 (dd, 1H), 8.08 (d, 1H), 7.81 (m, 2H), 7.33 (m, 1H), 7.19 (d, 1H), 4.40-4.30 (m, 2H), 3.75-3.55 (m, 2H), 1.53 (s, 3H), 1.45 (s, 9H); MS: 489 [(M+H)⁺].

### b) 5-Bromo-pyridine-2-carboxylic acid [3-(5-amino-3-methyl-3,6-dihydro-2H-[1,4]oxazin-3-yl)-phenyl]-amide hydrochloride

A solution of (5-{3-[(5-bromo-pyridine-2-carbonyl)-amino]-phenyl}-5-methyl-5,6-dihydro-2H-[1,4]oxazin-3-yl)-carbamic acid tert-butyl ester (44.4 mg, 0.1 mmol) in dichloromethane was treated with 4M HCl in dioxane (40 eq). The mixture was warmed to 40° C. for 10 h and then evaporated under reduced pressure to yield the title compound (hydrochloride salt) in the form of a colourless solid. ¹H-NMR (500 MHz, DMSO-d₆): 10.70 (s, 1H), 10.62 (s, 1H), 9.14 (s, 1H), 8.87 (d, 1H), 8.52 (s, 1H), 8.34 (dd, 1H), 8.11 (d, 1H), 7.96 (m, 2H), 7.43 (t, 1H), 7.21 (d, 1H), 4.59 (s, 2H), 3.88 (m, 2H), 1.65 (s, 3H); MS: 389 [(M+H)⁺].

### Examples 3 to 30

The compounds listed in Table 1 were prepared by procedures analogous to those used in examples 1 and 2.

TABLE 1

| | | | MS |
|---|---|---|---|
| | | ¹H-NMR | [m/z; |
| Example | Compound | (δ; DMSO-d₆) | (M + 1)⁺] |
| 3 | 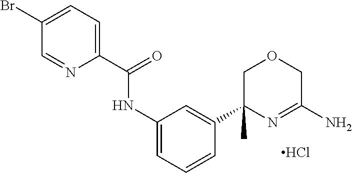 [SMILES: C[C@@]1(c2cccc(NC(=O)c3ccc(Br)cn3)c2)COCC(N)=N1 ; Cl] | 10.73 (s, 1H), 10.65 (s, 1H), 9.18 (s, 1H), 8.87 (d, 1H), 8.57 (s, 1H), 8.34 (dd, 1H), 8.11 (d, 1H), 7.96 (m, 2H), 7.43 (t, 1H), 7.22 (d, 1H), 4.59 (s, 2H), 3.88 (m, 2H), 1.65 (s, 3H) | 389 |
| | 5-Bromo-pyridine-2-carboxylic acid [3- | | |
| | ((R)-5-amino-3-methyl-3,6-dihydro-2H- | | |
| | [1,4]oxazin-3-yl)-phenyl]-amide | | |
| | hydrochloride | | |
| 4 | 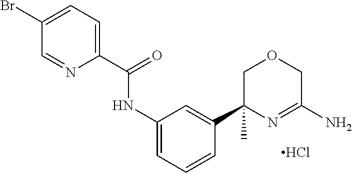 [SMILES: C[C@]1(c2cccc(NC(=O)c3ccc(Br)cn3)c2)COCC(N)=N1 ; Cl] | 10.73 (s, 1H), 10.65 (s, 1H), 9.18 (s, 1H), 8.87 (d, 1H), 8.57 (s, 1H), 8.34 (dd, 1H), 8.11 (d, 1H), 7.96 (m, 2H), 7.43 (t, 1H), 7.22 (d, 1H), 4.59 (s, 2H), 3.88 (m, 2H), 1.64 (s, 3H) | 389 |
| | 5-Bromo-pyridine-2-carboxylic acid [3- | | |
| | ((S)-5-amino-3-methyl-3,6-dihydro-2H- | | |
| | [1,4]oxazin-3-yl)-phenyl]-amide | | |
| | hydrochloride | | |
| 5 | 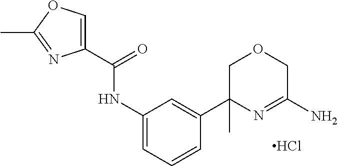 [SMILES: Cc1nc(C(=O)Nc2cccc(C3(C)COCC(N)=N3)c2)co1 ; Cl] | 9.91 (s, 1H), 8.61 (s, 1H), 7.76 (s, 1H), 7.65 (d, 1H), 7.23 (t, 1H), 7.16 (d, 1H), 5.57 (br, 2H), 4.00-3.85 (m, 2H), 3.60-3.40 (m, 2H), 1.34 (s, 3H) | 315 |
| | 2-Methyl-oxazole-4-carboxylic acid [3- | | |
| | (5-amino-3-methyl-3,6-dihydro-2H- | | |
| | [1,4]oxazin-3-yl)-phenyl]-amide | | |
| | hydrochloride | | |
| 6 | 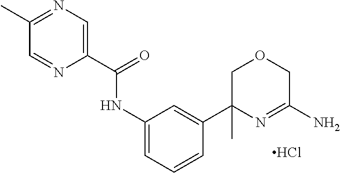 [SMILES: Cc1cnc(C(=O)Nc2cccc(C3(C)COCC(N)=N3)c2)cn1 ; Cl] | 10.51 (s, 1H), 9.16 (s, 1H), 8.70 (s, 1H), 7.87 (s, 1H), 7.76 (d, 1H), 7.29 (t, 1H), 7.21 (d, 1H), 5.76 (br, 2H), 4.00-3.85 (m, 2H), 3.63-3.48 (m, 2H), 1.37 (s, 3H) | 326 |
| | 5-Methyl-pyrazine-2-carboxylic acid [3- | | |
| | (5-amino-3-methyl-3,6-dihydro-2H- | | |
| | [1,4]oxazin-3-yl)-phenyl]-amide | | |
| | hydrochloride | | |
| 7 | 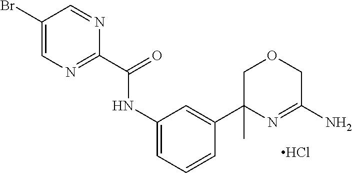 [SMILES: CC1(c2cccc(NC(=O)c3ncc(Br)cn3)c2)COCC(N)=N1 ; Cl] | 10.85 (s, 1H), 10.65 (s, 1H), 9.24 (s, 2H), 9.16 (s, 1H), 8.55 (s, 1H), 7.90 (m, 2H), 7.44 (t, 1H), 7.23 (d, 1H), 4.59 (s, 2H), 3.91 (m, 2H), 1.65 (s, 3H) | 390 |
| | 5-Bromo-pyrimidine-2-carboxylic acid | | |
| | [3-(5-amino-3-methyl-3,6-dihydro-2H- | | |
| | [1,4]oxazin-3-yl)-phenyl]-amide | | |
| | hydrochloride | | |
| 8 | 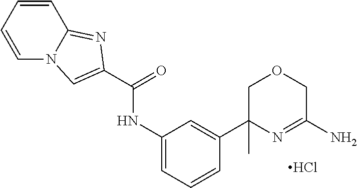 [SMILES: CC1(c2cccc(NC(=O)c3cn4ccccc4n3)c2)COCC(N)=N1 ; Cl] | 10.70 (s, 2H), 9.19 (s, 1H), 8.77 (m, 2H), 8.57 (s, 1H), 7.94 (m, 2H), 7.73 (d, 1H), 7.60 (t, 1H), 7.43 (t, 1H), 7.21 (d, 2H), 4.59 (s, 2H), 3.89 (m, 2H), 1.65 (s, 3H) | 350 |
| | Imidazo[1,2-a]pyridine-2-carboxylic acid | | |
| | [3-(5-amino-3-methyl-3,6-dihydro-2H- | | |
| | [1,4]oxazin-3-yl)-phenyl]-amide | | |
| | hydrochloride | | |
| 9 | 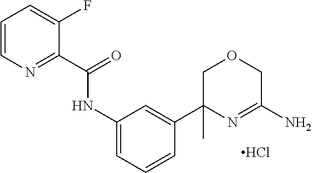 [SMILES: CC1(c2cccc(NC(=O)c3ncccc3F)c2)COCC(N)=N1 ; Cl] | 10.69 (s, 1H), 10.65 (s, 1H), 9.15 (s, 1H), 8.57 (d, 1H), 8.53 (s, 1H), 7.95 (t, 1H), 7.87 (m, 2H), 7.76 (m, 1H), 7.43 (t, 1H), 7.22 (d, 1H), 4.59 (s, 2H), 3.91 (m, 2H), 1.64 (s, 3H) | 329 |
| | 3-Fluoro-pyridine-2-carboxylic acid [3- | | |
| | (5-amino-3-methyl-3,6-dihydro-2H- | | |
| | [1,4]oxazin-3-yl)-phenyl]-amide | | |
| | hydrochloride | | |
| 10 | 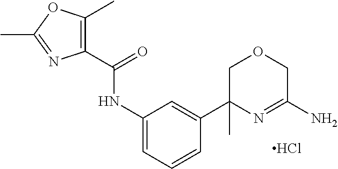 [SMILES: Cc1nc(C(=O)Nc2cccc(C3(C)COCC(N)=N3)c2)c(C)o1 ; Cl] | 10.60 (s, 1H), 9.98 (s, 1H), 9.12 (s, 1H), 8.49 (s, 1H), 7.87 (m, 2H), 7.37 (t, 1H), 7.16 (d, 1H), 4.58 (s, 2H), 3.88 (m, 2H), 2.59 (s, 3H), 2.46 (s, 3H), 1.63 (s, 3H) | 329 |
| | 2,5-Dimethyl-oxazole-4-carboxylic acid | | |
| | [3-(5-amino-3-methyl-3,6-dihydro-2H- | | |
| | [1,4]oxazin-3-yl)-phenyl]-amide | | |
| | hydrochloride | | |
| 11 | 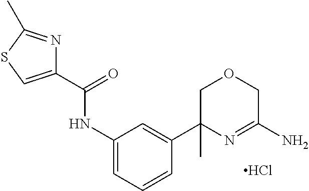 [SMILES: Cc1nc(C(=O)Nc2cccc(C3(C)COCC(N)=N3)c2)cs1 ; Cl] | 10.63 (s, 1H), 10.24 (s, 1H), 9.13 (s, 1H), 8.51 (s, 1H), 8.29 (s, 1H), 7.89 (m, 1H), 7.40 (t, 1H), 7.18 (d, 1H), 4.58 (s, 2H), 3.88 (m, 2H), 2.77 (s, 3H), 1.64 (s, 3H) | 331 |
| | 2-Methyl-thiazole-4-carboxylic acid [3- | | |
| | (5-amino-3-methyl-3,6-dihydro-2H- | | |
| | [1,4]oxazin-3-yl)-phenyl]-amide | | |
| | hydrochloride | | |
| 12 | 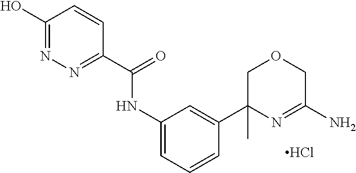 [SMILES: CC1(c2cccc(NC(=O)c3ccc(O)nn3)c2)COCC(N)=N1 ; Cl] | 13.57 (s, 1H), 10.60 (s, 1H), 10.38 (s, 1H), 9.14 (s, 1H), 8.52 (s, 1H), 7.92 (d, 1H), 7.83 (m, 2H), 7.41 (t, 1H), 7.20 (d, 1H), 7.04 (d, 1H), 4.58 (s, 2H), 3.89 (m, 2H), 1.63 (s, 3H) | 328 |
| | 6-Hydroxy-pyridazine-3-carboxylic acid | | |
| | [3-(5-amino-3-methyl-3,6-dihydro-2H- | | |
| | [1,4]oxazin-3-yl)-phenyl]-amide | | |
| | hydrochloride | | |
| 13 | 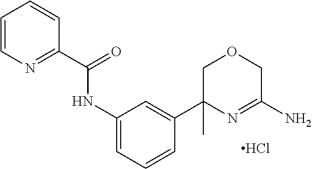 [SMILES: CC1(c2cccc(NC(=O)c3ccccn3)c2)COCC(N)=N1 ; Cl] | 10.71 (s, 1H), 10.65 (s, 1H), 9.16 (s, 1H), 8.75 (d, 1H), 8.54 (s, 1H), 8.17 (d, 1H), 8.09 (t, 1H), 7.97 (m, 2H), 7.70 (m, 1H), 7.43 (t, 1H), 7.21 (d, 1H), 4.59 (s, 2H), 3.91 (m, 2H), 1.65 (s, 3H) | 311 |
| | Pyridine-2-carboxylic acid [3-(5-amino- | | |
| | 3-methyl-3,6-dihydro-2H-[1,4]oxazin-3- | | |
| | yl)-phenyl]-amide hydrochloride | | |
| 14 | 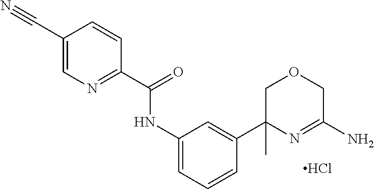 [SMILES: CC1(c2cccc(NC(=O)c3ccc(C#N)cn3)c2)COCC(N)=N1 ; Cl] | 10.88 (s, 1H), 10.62 (s, 1H), 9.22 (s, 1H), 9.14 (s, 1H), 8.59 (m, 1H), 8.53 (s, 1H), 8.31 (d, 1H), 7.96 (m, 2H), 7.44 (t, 1H), 7.23 (d, 1H), 4.59 (s, 2H), 3.88 (m, 2H), 1.65 (s, 3H) | 336 |
| | 5-Cyano-pyridine-2-carboxylic acid [3- | | |
| | (5-amino-3-methyl-3,6-dihydro-2H- | | |
| | [1,4]oxazin-3-yl)-phenyl]-amide | | |
| | hydrochloride | | |
| 15 | 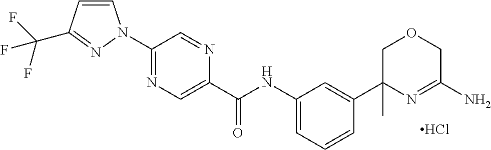 [SMILES: CC1(c2cccc(NC(=O)c3cnc(-n4ccc(C(F)(F)F)n4)cn3)c2)COCC(N)=N1 ; Cl] | 10.90 (s, 1H), 10.60 (s, 1H), 9.28 (s, 1H), 9.20 (s, 1H), 9.10 (s, 1H), 8.96 (s, 1H), 8.50 (s, 1H), 7.94 (m, 2H), 7.40 (t, 1H), 7.21 (m, 2H), 4.59 (s, 2H), 3.88 (m, 2H), 1.64 (s, 3H) | 446 |
| | 5-(3-Trifluoromethyl-pyrazol-1-yl)- | | |
| | pyrazine-2-carboxylic acid [3-(5-amino- | | |
| | 3-methyl-3,6-dihydro-2H-[1,4]oxazin-3- | | |
| | yl)-phenyl]-amide hydrochloride | | |
| 16 | 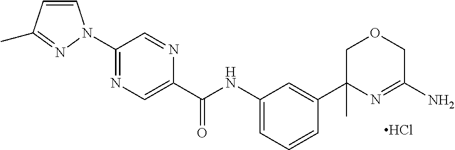 [SMILES: Cc1ccn(-c2cnc(C(=O)Nc3cccc(C4(C)COCC(N)=N4)c3)cn2)n1 ; Cl] | 10.80 (s, 1H), 10.60 (s, 1H), 9.12 (m, 3H), 8.60 (s, 1H), 8.50 (s, 1H), 7.94 (m, 2H), 7.45 (t, 1H), 7.21 (d, 1H), 6.55 (s, 1H), 4.59 (s, 2H), 3.88 (m, 2H), 2.35 (s, 3H), 1.64 (s, 3H) | 392 |
| | 5-(3-Methyl-pyrazol-1-yl)-pyrazine-2- | | |
| | carboxylic acid [3-(5-amino-3-methyl- | | |
| | 3,6-dihydro-2H-[1,4]oxazin-3-yl)- | | |
| | phenyl]-amide hydrochloride | | |
| 17 | 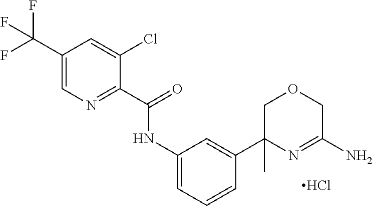 [SMILES: CC1(c2cccc(NC(=O)c3ncc(C(F)(F)F)cc3Cl)c2)COCC(N)=N1 ; Cl] | 10.91 (s, 1H), 10.60 (br, 1H), 9.11 (br, 1H), 9.07 (s, 1H), 8.72 (s, 1H), 8.50 (br, 1H), 7.76 (m, 2H), 7.46 (t, 1H), 7.26 (d, 1H), 4.58 (s, 2H), 3.89 (m, 2H), 1.64 (s, 3H) | 413 |
| | 3-Chloro-5-trifluoromethyl-pyridine-2- | | |
| | carboxylic acid [3-(5-amino-3-methyl- | | |
| | 3,6-dihydro-2H-[1,4]oxazin-3-yl)- | | |
| | phenyl]-amide hydrochloride | | |
| 18 | 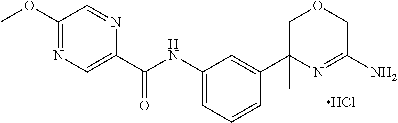 [SMILES: COc1cnc(C(=O)Nc2cccc(C3(C)COCC(N)=N3)c2)cn1 ; Cl] | 10.60 (s, 1H), 9.16 (s, 1H), 8.90 (s, 1H), 8.50 (s, 1H), 8.42 (s, 1H), 7.94 (m, 2H), 7.40 (t, 1H), 7.21 (d, 1H), 4.59 (s, 2H), 4.05 (s, 3H), 3.88 (m, 2H), 1.64 (s, 3H) | 342 |
| | 5-Methoxy-pyrazine-2-carboxylic acid | | |
| | [3-(5-amino-3-methyl-3,6-dihydro-2H- | | |
| | [1,4]oxazin-3-yl)-phenyl]-amide | | |
| | hydrochloride | | |
| 19 | 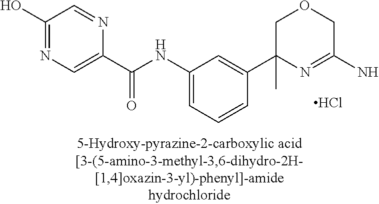 [SMILES: CC1(c2cccc(NC(=O)c3cnc(O)cn3)c2)COCC(N)=N1 ; Cl] | 13.00 (s, 1H), 10.60 (s, 1H), 10.25 (s, 1H), 9.16 (s, 1H), 8.48 (s, 1H), 8.10 (br, 1H), 8.05 (s, 1H), 7.91 (d, 1H), 7.85 (s, 1H), 7.38 (t, 1H), 7.15 (d, 1H), 4.59 (s, 2H), 3.88 (m, 2H), 1.64 (s, 3H) | 328 |
| 20 | 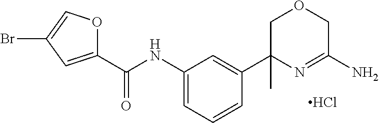 [SMILES: CC1(c2cccc(NC(=O)c3cc(Br)co3)c2)COCC(N)=N1 ; Cl] | 10.65 (s, 1H), 10.42 (s, 1H), 9.14 (s, 1H), 8.52 (s, 1H), 8.22 (s, 1H), 7.80 (s, 1H), 7.75 (d, 1H), 7.56 (s, 1H), 7.40 (m, 2H), 7.18 (d, 1H), 4.59 (s, 2H), 3.87 (m, 2H), 1.64 (s, 3H) | 379 |
| | 4-Bromo-furan-2-carboxylic acid [3-(5- | | |
| | amino-3-methyl-3,6-dihydro-2H- | | |
| | [1,4]oxazin-3-yl)-phenyl]-amide | | |
| | hydrochloride | | |
| 21 | 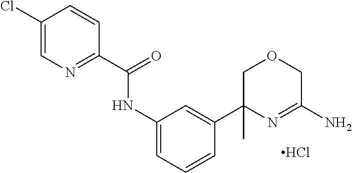 [SMILES: CC1(c2cccc(NC(=O)c3ccc(Cl)cn3)c2)COCC(N)=N1 ; Cl] | 10.71 (s, 1H), 10.60 (s, 1H), 9.25 (s, 1H), 8.79 (s, 1H), 8.52 (s, 1H), 8.18 (m, 2H), 7.96 (m, 2H), 7.42 (t, 1H), 7.20 (d, 1H), 4.58 (s, 2H), 3.89 (m, 2H), 1.64 (s, 3H) | 345 |
| | 5-Chloro-pyridine-2-carboxylic acid [3- | | |
| | (5-amino-3-methyl-3,6-dihydro-2H- | | |
| | [1,4]oxazin-3-yl)-phenyl]-amide | | |
| | hydrochloride | | |
| 22 | 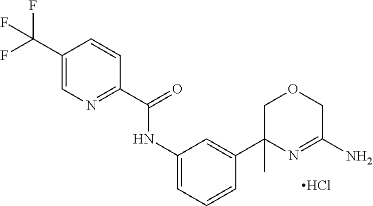 [SMILES: CC1(c2cccc(NC(=O)c3ccc(C(F)(F)F)cn3)c2)COCC(N)=N1 ; Cl] | 10.81 (s, 1H), 10.60 (br, 1H), 9.11 (s, 1H), 8.51 (d, 1H), 8.34 (d, 1H), 7.95 (m, 2H), 7.44 (m, 2H), 7.22 (d, 1H), 4.58 (s, 2H), 3.89 (m, 2H), 1.64 (s, 3H) | 379 |
| | 5-Trifluoromethyl-pyridine-2-carboxylic | | |
| | acid [3-(5-amino-3-methyl-3,6-dihydro- | | |
| | 2H-[1,4]oxazin-3-yl)-phenyl]-amide | | |
| | hydrochloride | | |
| 23 | 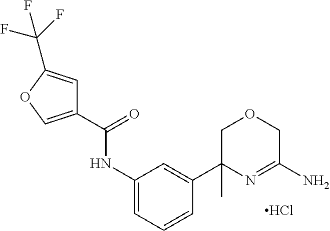 [SMILES: CC1(c2cccc(NC(=O)c3coc(C(F)(F)F)c3)c2)COCC(N)=N1 ; Cl] | 10.60 (s, 1H), 10.35 (s, 1H), 9.11 (s, 1H), 8.72 (s, 1H), 8.48 (s, 1H), 7.80 (s, 2H), 7.70 (d, 1H), 7.43 (m, 2H), 7.21 (d, 1H), 4.58 (s, 2H), 3.88 (m, 2H), 1.64 (s, 3H) | 368 |
| | 5-Trifluoromethyl-furan-3-carboxylic | | |
| | acid [3-(5-amino-3-methyl-3,6-dihydro- | | |
| | 2H-[1,4]oxazin-3-yl)-phenyl]-amide | | |
| | hydrochloride | | |
| 24 | 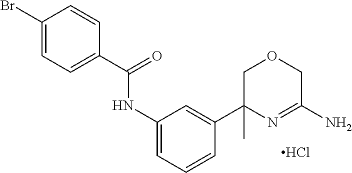 [SMILES: CC1(c2cccc(NC(=O)c3ccc(Br)cc3)c2)COCC(N)=N1 ; Cl] | 10.60 (s, 1H), 10.44 (s, 1H), 9.12 (s, 1H), 8.50 (s, 1H), 7.91 (m, 2H), 7.84 (s, 1H), 7.77 (m, 3H), 7.42 (t, 1H), 7.20 (d, 1H), 4.59 (s, 2H), 3.88 (m, 2H), 1.64 (s, 3H) | 388 |
| | N-[3-(5-Amino-3-methyl-3,6-dihydro- | | |
| | 2H-[1,4]oxazin-3-yl)-phenyl]-4-bromo- | | |
| | benzamide hydrochloride | | |
| Reference Example 25 | 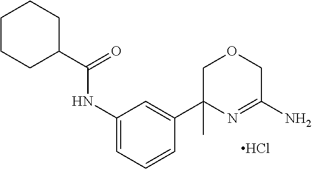 [SMILES: CC1(c2cccc(NC(=O)C3CCCCC3)c2)COCC(N)=N1 ; Cl] | 10.60 (s, 1H), 9.96 (s, 1H), 9.15 (s, 1H), 8.53 (s, 1H), 7.72 (s, 1H), 7.56 (m, 1H), 7.33 (t, 1H), 7.09 (d, 1H), 4.57 (s, 2H), 3.86 (m, 2H), 2.34 (m, 1H), 1.80-1.15 (m, 10H), 1.60 (s, 3H) | 316 |
| | Cyclohexanecarboxylic acid [3-(5- | | |
| | amino-3-methyl-3,6-dihydro-2H- | | |
| | [1,4]oxazin-3-yl)-phenyl]-amide | | |
| | hydrochloride | | |
| Reference Example 26 | 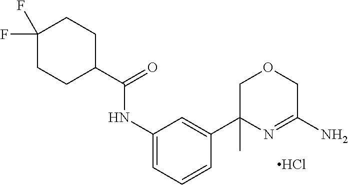 [SMILES: CC1(c2cccc(NC(=O)C3CCC(F)(F)CC3)c2)COCC(N)=N1 ; Cl] | 10.55 (s, 1H), 10.11 (s, 1H), 9.09 (s, 1H), 8.46 (d, 1H), 7.73 (s, 1H), 7.54 (d, 1H), 7.35 (t, 1H), 7.12 (d, 1H), 4.57 (s, 2H), 3.85 (m, 2H), 2.09 (m, 2H), 1.91 (m, 4H), 1.82 (m, 2H), 1.69 (m, 2H), 1.61 (s, 3H) | 352 |
| | 4,4-Difluoro-cyclohexanecarboxylic acid | | |
| | [3-(5-amino-3-methyl-3,6-dihydro-2H- | | |
| | [1,4]oxazin-3-yl)-phenyl]-amide | | |
| | hydrochloride | | |
| 27 | 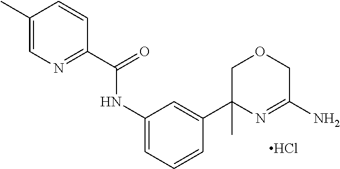 [SMILES: Cc1ccc(C(=O)Nc2cccc(C3(C)COCC(N)=N3)c2)nc1 ; Cl] | 10.70 (s, 1H), 10.65 (s, 1H), 9.18 (s, 1H), 8.57 (m, 2H), 8.07 (m, 1H), 7.98 (d, 1H), 7.95 (s, 1H), 7.90 (m, 1H), 7.42 (t, 2H), 7.20 (d, 1H), 4.59 (s, 2H), 3.88 (m, 2H), 2.43 (s, 3H), 1.65 (s, 3H) | 325 |
| | 5-Methyl-pyridine-2-carboxylic acid [3- | | |
| | (5-amino-3-methyl-3,6-dihydro-2H- | | |
| | [1,4]oxazin-3-yl)-phenyl]-amide | | |
| | hydrochloride | | |
| 28 | 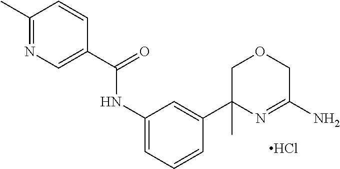 [SMILES: Cc1ccc(C(=O)Nc2cccc(C3(C)COCC(N)=N3)c2)cn1 ; Cl] | 10.74 (s, 1H), 9.22 (s, 1H), 9.20 (s, 1H), 8.85 (d, 1H), 8.58 (s, 1H), 8.49 (d, 1H), 7.86 (s, 1H), 7.79 (m, 1H), 7.72 (m, 1H), 7.44 (t, 1H), 7.23 (d, 1H), 4.59 (s, 2H), 3.91 (m, 2H), 1.65 (s, 3H) | 311 |
| | N-[3-(5-Amino-3-methyl-3,6-dihydro- | | |
| | 2H-[1,4]oxazin-3-yl)-phenyl]- | | |
| | nicotinamide hydrochloride | | |
| 29 | 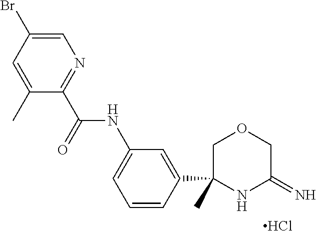 [SMILES: Cc1cc(Br)cnc1C(=O)Nc1cccc([C@]2(C)COCC(=N)N2)c1 ; Cl] | 10.74 (s, 1H), 10.63 (s, 1H), 9.20 (s, 1H), 8.68 (1 s, 1H), 8.59 (s, 1H), 8.18 (s, 1H), 7.88 (d, 1H), 7.81 (s, 1H), 7.41 (t, 1H), 7.19 (d, 1H), 4.59 (s, 2H), 3.88 (m, 2H(AB-sytem)), 2.58 (s, 3H), 1.65 (s, 3H) | 403 |
| | 5-Bromo-3-methyl-pyridine-2-carboxylic | | |
| | acid [3-((R)-5-amino-3-methyl-3,6- | | |
| | dihydro-2H-[1,4]oxazin-3-yl)-phenyl]- | | |
| | amide hydrochloride | | |
| 30 | 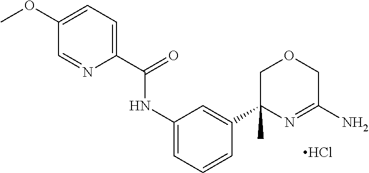 [SMILES: COc1ccc(C(=O)Nc2cccc([C@]3(C)COCC(N)=N3)c2)nc1 ; Cl] | 10.69 (s, 1H), 10.55 (br, 1H), 9.15 (s, 1H), 8.55 (d, 1H), 8.39 (d, 1H), 8.14 (d, 1H), 7.96 (d, 1H), 7.92 (s, 1H), 7.64 (dd, 1H), 7.41 (t, 1H), 7.17 (d, 1H), 4.58 (s, 2H), 3.96 (s, 3H), 3.90 (m, 2H), 1.64 (s, 3H) | 341 |
| | 5-Methoxy-pyridine-2-carboxylic acid | | |
| | [3-((R)-5-amino-3-methyl-3,6-dihydro- | | |
| | 2H-[1,4]oxazin-3-yl)-phenyl]-amide | | |
| | hydrochloride | | |

The racemic (5-{3-[(5-bromo-pyridine-2-carbonyl)-amino]-phenyl}-5-methyl-5,6-dihydro-2H-[1,4]oxazin-3-yl)-carbamic acid tert-butyl ester was separated into the pure enantiomers by preparative chiral HPLC (column: CHIRACEL OD-PREP; solvent: heptane/ethanol/methanol=90:5:5; flow: 1 ml/min; detection at 210 nm). These enantiomers were treated with 4M HCl in dioxane to obtain the enantiomerically pure compounds 3 and 4. Example 3: [α]_{D}=−50.0°, c=0.519% (MeOH). Example 4: [α]_{D}=+ 58.1°, c=0.498% (MeOH). Examples 29 and 30 can be obtained by a similar procedure.

### Example 31

### 5-Bromo-pyridine-2-carboxylic acid [3-(5-amino-3-fluoromethyl-3,6-dihydro-2H-[1,4]oxazin-3-yl)-phenyl]-amide

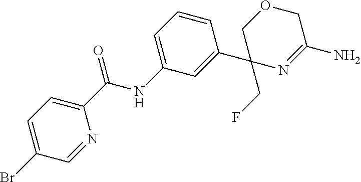 [SMILES: NC1=NC(CF)(c2cccc(NC(=O)c3ccc(Br)cn3)c2)COC1]

### a) 2-(3-Bromo-phenyl)-2-nitro-propane-1,3-diol

A mixture of 1-bromo-3-nitromethyl-benzene (6.82 g, 31.6 mmol), formaline (35%, 5.22 ml, 66.3 mmol) and Et₃N (2.2 ml, 15.78 mmol) was heated at 50° C. for 1 h, diluted with water and extracted with TBME. The organic phase was washed with brine, dried with MgSO₄ and evaporated. The residue was crystallized from TBME/hexane to yield the title compound in the form of a colourless solid. TLC (hexane/EtOAc=2:1): R_{f}=0.2; HPLC: Rt_{H2}=3.117 min; ¹H-NMR (400 MHz, CD₃OD): 7.61-7.54 (m, 2H), 7.39-7.34 (m, 2H), 4.40 (d, 2H), 4.35 (d, 2H); MS: 298, 300 [(M+Na)⁺].

### b) 2-Amino-2-(3-bromo-phenyl)-propane-1,3-diol

A solution of 2-(3-bromo-phenyl)-2-nitro-propane-1,3-diol (6.79 g, 24.59 mmol) in 100 ml of EtOH was hydrogenated in the presence of 5 g of Raney-Ni. When the take-up of hydrogen had ceased, the mixture was filtered through Celite, and the filtrate was chromatographed on silica gel (EtOAc/MeOH/25% aqueous NH₃, 5%) to give the title compound in the form of a colourless solid. TLC (EtOAc/MeOH/25% aqueous NH₃, 5%). R_{f}=0.24; HPLC: Rt_{H2}=2.354 min; ¹H-NMR (400 MHz, CD₃OD): 7.73 (s, 1H), 7.50 (d, 1H), 7.44 (d, 1H), 7.29 (t, 1H), 3.79 (d, 2H), 3.72 (d, 2H); MS: 246, 248 [(M+H)⁺].

### c) N-[1-(3-Bromo-phenyl)-2-hydroxy-1-hydroxymethyl-ethyl]-2-chloro-acetamide

To a stirred suspension of 2-amino-2-(3-bromo-phenyl)-propane-1,3-diol (3.5 g, 14.22 mmol), 30 ml of THF and 30 ml of 10% aqueous Na₂CO₃ solution was added dropwise chloroacetyl chloride (1.472 ml, 18.5 mmol) at 0° C. over a period of 10 min. The mixture was stirred for 1 h, diluted with water and extracted with EtOAc. The organic phase was washed with 1N aqueous NaOH solution, 10% aqueous Na₂CO₃ solution and brine. Chromatography on silica gel (EtOAc/hexane 50-30%) gave the title compound in the form of a colourless solid. TLC (hexane/EtOAc=1:1): R_{f}=0.21; HPLC: Rt_{H2}=2.926 min; ¹H-NMR (400 MHz, CD₃OD): 7.58 (s, 1H), 7.44 (d, 1H), 7.37 (d, 1H), 7.29 (t, 1H), 4.91 (s, 2H), 4.07 (d, 2H), 4.00 (d, 2H); MS: 322, 324, 326 [(M+H)⁺].

### d) 5-(3-Bromo-phenyl)-5-hydroxymethyl-morpholin-3-one

A suspension of N-[1-(3-bromo-phenyl)-2-hydroxy-1-hydroxymethyl-ethyl]-2-chloro-acetamide (3.24 g, 10.04 mmol) and 35 ml of t-BuOH was treated with potassium tert-butoxide (1.127 g, 10.04 mmol). The mixture was heated at reflux for 1 h and neutralized with 10 ml of 1N HCl. Water and TBME were added, and the precipitate was filtered off. The organic phase of the filtrate was separated, dried with sodium sulfate and chromatographed on silica gel (EtOAc/MeOH 1-2%) to give the title compound in the form of a colourless solid. TLC (EtOAc/MeOH 1%). R_{f}=0.23; HPLC: Rt_{H2}=2.839 min; ¹H-NMR (400 MHz, CD₃OD): 7.70 (s, 1H), 7.53-7.47 (m, 2H), 7.35 (t, 1H), 4.17 (s, 2H), 4.08 (d, 1H), 3.98 (d, 1H), 3.91 (d, 1H), 3.87 (d, 1H); MS: 287, 289 [(M+H)⁺].

### e) 5-(3-Bromo-phenyl)-5-fluoromethyl-morpholin-3-one

A suspension of 5-(3-bromo-phenyl)-5-hydroxymethyl-morpholin-3-one (2.6 g, 9.09 mmol) and 120 ml of dichloromethane was cooled to 0° C. DAST (1.26 ml) was added dropwise. The mixture was stirred overnight, poured onto 50 ml of 10% aqueous Na₂CO₃ solution and ice and extracted with dichloromethane. The extract was dried with sodium sulfate and evaporated. Chromatography on silica gel (EtOAc/hexane=1:1) gave the title compound in the form of a colourless solid. TLC (hexane/EtOAc=1:1): R_{f}=0.31; HPLC: Rt_{H2}=3.13 min; ¹H-NMR (400 MHz, CD₃OD): 7.71 (s, 1H), 7.56 (d, 1H), 7.52 (d, 1H), 7.38 (t, 1H), 4.77 (d, 2H), 4.20 (s, 2H), 4.11 (d, 1H), 3.95 (d, 1H); MS: 289, 291 [(M+H)⁺].

### f) 5-(3-Bromo-phenyl)-5-fluoromethyl-morpholine-3-thione

A mixture of 5-(3-bromo-phenyl)-5-fluoromethyl-morpholin-3-one (1.52 g, 5.28 mmol) and Lawesson's reagent (2.14 g, 5.28 mmol) in 21 ml of THF was heated at 50° C. for 1 h and then evaporated. The residue was chromatographed on silica gel (cyclohexane/EtOAc=15:1) to give the title compound in the form of a colourless foam. TLC (hexane/EtOAc=3:1): R_{f}=0.21; HPLC: Rt_{H2}=3.49 min; ¹H-NMR (400 MHz, CD₃OD): 7.65 (s, 1H), 7.58 (d, 1H), 7.47 (d, 1H), 7.39 (t, 1H), 4.87 (s, 2H), 4.63 (d, 1H), 4.50 (s, 2H), 4.02 (d, 1H); MS: 304, 306 [(M+H)⁺].

### g) [5-(3-Bromo-phenyl)-5-fluoromethyl-5,6-dihydro-2H-[1,4]oxazin-3-yl]-carbamic acid tert-butyl ester

To a solution of 5-(3-bromo-phenyl)-5-fluoromethyl-morpholine-3-thione (200 mg, 0.658 mmol) in 5 ml of 7M NH₃/MeOH were added t-butyl hydroperoxide (80%, 0.818 ml, 6.58 mmol) and then 1.7 ml of 25% aqueous NH₄OH. After 2 h, the mixture was quenched with a saturated aqueous solution of Na₂S₂O₃ and extracted with EtOAc. The extract was washed with brine, dried with sodium sulfate and evaporated. The crude 5-(3-bromo-phenyl)-5-fluoromethyl-5,6-dihydro-2H-[1,4]oxazin-3-ylamine (189 mg, 0.658 mmol) was dissolved in 4 ml of dichloromethane. The solution was treated with DIPEA (0.172 ml, 0.987 mmol) and Boc₂O (187 mg, 0.855 mmol). After 14 h, the mixture was diluted with dichloromethane and washed with water, 1N HCl and brine. The organic phase was dried with sodium sulfate and evaporated. The residue was chromatographed on silica gel (cyclohexane/EtOAc=6:1) to yield the title compound. TLC (hexane/EtOAc=6:1): R_{f}=0.20; HPLC: Rt_{H1}=2.380 min; ¹H-NMR (400 MHz, CDCl₃): 7.56-6.98 (m, 4H; broad signals due to rotamers), 4.80-3.60 (m, 6H), 1.42 (br, 9H); MS: 387, 389 [(M+H)⁺].

### h) [5-(3-Azido-phenyl)-5-fluoromethyl-5,6-dihydro-2H-[1,4]oxazin-3-yl]-carbamic acid tert-butyl ester

A suspension of [5-(3-bromo-phenyl)-5-fluoromethyl-5,6-dihydro-2H-[1,4]oxazin-3-yl]-carbamic acid tert-butyl ester (114 mg, 0.295 mmol), NaN₃ (77 mg, 1.18 mmol), CuI (11 mg, 0.059 mmol), sodium ascorbate (12 mg, 0.059 mmol), N,N′-dimethyl-cyclohexane-1,2-diamine (13 mg, 0.089 mmol), 1.5 ml of EtOH and 0.6 ml of water was stirred under N₂ at 90° C. for 1 h. The mixture was filtered through Celite, and the filtrate was chromatographed on silica gel (cyclohexane/EtOAc=6:1) to yield the title compound in the form of a colourless foam. TLC (hexane/EtOAc=3:1): R_{f}=0.33; HPLC: Rt_{H1}=2.258 min; ¹H-NMR (400 MHz, CDCl₃): 7.40-6.88 (m, 4H; broad signals due to rotamers), 4.75-3.60 (m, 6H), 1.42 (br, 9H); MS: 350 [(M+H)⁺].

### i) (5-{3-[(5-Bromo-pyridine-2-carbonyl)-amino]-phenyl}-5-fluoromethyl-5,6-dihydro-2H-[1,4]oxazin-3-yl)-carbamic acid tert-butyl ester

A solution of [5-(3-azido-phenyl)-5-fluoromethyl-5,6-dihydro-2H-[1,4]oxazin-3-yl]-carbamic acid tert-butyl ester (56 mg, 0.161 mmol) in 2 ml of EtOAc was hydrogenated in the presence of Lindlar catalyst (11 mg) for 3 h. The mixture was filtered through Celite, and the filtrate was evaporated. The crude [5-(3-amino-phenyl)-5-fluoromethyl-5,6-dihydro-2H-[1,4]oxazin-3-yl]-carbamic acid tert-butyl ester (50 mg, 0.155 mmol) was taken up in 2 ml of dichloromethane. A mixture of 5-bromo-pyridine-2-carboxylic acid (34.4 mg, 0.170 mmol), HOBT (30.9 mg, 0.17 mmol) and EDC (32.6 mg, 0.17 mmol) in 2 ml of dichloromethane was added, followed by the addition of triethylamine (0.054 ml). The mixture was stirred for 4 h, treated with 5% aqueous NaHCO₃ solution and extracted twice with dichloromethane. The organic phase was dried with MgSO₄ and evaporated. The residue was chromatographed on silica gel (EtOAc/cyclohexane=1:4) to yield the title compound. TLC (hexane/EtOAc=3:1): R_{f}=0.16; HPLC: Rt_{H1}=2.763 min; ¹H-NMR (400 MHz, CDCl₃): 9.80 (br, 1H), 8.60 (d, 1H), 8.11 (d, 1H), 7.98 (d, 1H), 7.77 (br, 1H), 7.73 (d, 1H), 7.33 (br, 1H), 7.15 (d, 1H), 4.75-3.65 (m, 6H), 1.60 (br; minor rotamer tBu), 1.42 (br; major rotamer tBu); MS: 507, 509 [(M+H)⁺].

### j) 5-Bromo-pyridine-2-carboxylic acid [3-(5-amino-3-fluoromethyl-3,6-dihydro-2H-[1,4]oxazin-3-yl)-phenyl]-amide

A solution of (5-{3-[(5-bromo-pyridine-2-carbonyl)-amino]-phenyl}-5-fluoromethyl-5,6-dihydro-2H-[1,4]oxazin-3-yl)-carbamic acid tert-butyl ester (48 mg, 0.095 mmol) in 2 ml of 3N HCl in MeOH was stirred at 40° C. for 2 h. The mixture was evaporated, and the residue was purified by chromatography on silica gel with a gradient of dichloromethane and 2-10% MeOH/NH₄OH (0.5%), yielding the title compound in the form of a colorless foam. TLC (dichloromethane/MeOH/25% aqueous NH₄OH=90:9:1): R_{f}=0.28; HPLC: Rt_{H1}=2.755 min; ¹H-NMR (400 MHz, CDCl₃): 8.58 (d, 1H), 8.10 (d, 1H), 7.96 (dd, 1H), 7.76 (s, 1H), 7.71 (d, 1H), 7.31 (t, 1H), 7.21 (d, 1H), 4.55-4.33 (m, 2H), 4.13-3.95 (m, 3H), 3.65 (d, 1H), 4.0-3.3 (br, NH₂); MS: 407, 409 [(M+H)⁺].

### Example 32

The compound listed in Table 2 was prepared by a procedure analogous to that used in example 31 starting from 1-bromo-3-chloro-5-nitromethyl-benzene.

TABLE 2

| | | | MS |
|---|---|---|---|
| | | ¹H-NMR | [m/z; |
| Example | Compound | (δ; CD₃OD) | (M + 1)⁺] |
| 32 | 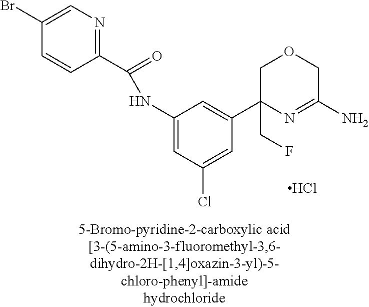 [SMILES: NC1=NC(CF)(c2cc(Cl)cc(NC(=O)c3ccc(Br)cn3)c2)COC1 ; Cl] | 8.85 (d, 1H), 8.29 (m, 1H), 8.17 (d, 1H), 8.07 (m, 1H), 7.97 (s, 1H), 7.37 (s, 2H), 5.05 (m, 1H), 4.95 (m, 1H), 4.70 (s, 2H), 4.19 (m, 2H) | 442 |

### Example 33

### 5-Bromo-pyridine-2-carboxylic acid [3-((R)-5-amino-3-fluoromethyl-3,6-dihydro-2H-[1,4]oxazin-3-yl)-phenyl]-amide

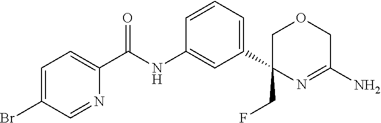 [SMILES: NC1=N[C@](CF)(c2cccc(NC(=O)c3ccc(Br)cn3)c2)COC1]

### a) (R)-[5-(3-Amino-phenyl)-5-fluoromethyl-5,6-dihydro-2H-[1,4]oxazin-3-yl]-carbamic acid tert-butyl ester

A solution of 740 mg (2.118 mmol) [5-(3-azido-phenyl)-5-fluoromethyl-5,6-dihydro-2H-[1,4]oxazin-3-yl]-carbamic acid tert-butyl ester (Example 31h) in 5 ml THF and 5 ml EtOH was stirred in the presence of 35 mg 10% Pd—C under hydrogen. After 3 h the mixture was filtered over celite, concentrated and crystallized from EtOAc/hexane to give a beige solid. The racemic product was separated via prep HPLC on Chiralpak AD-H 250×4.6 mm column using heptan/EtOH 1:1 as an eluent. The desired compound was the slower eluting (R)-enantiomer. TLC: Rf (Hexane/EtOAc 2:1)=0.15. HPLC: Rt_{H4}=1.764 min; ESIMS [M+H]⁺=324. ¹H-NMR (CDCl3, 360 MHz, broad signals due to hindered rotation): 10.5 (br, 1H), 7.12 (br, 1H), 6.69 (d, 1H), 6.59 (br d, 1H), 4.8-4.0 (m, 8H), 1.48 (br s, 9H).

### b) ((R)-5-{3-[(5-Bromo-pyridine-2-carbonyl)-amino]-phenyl}-5-fluoromethyl-5,6-dihydro-2H-[1,4]oxazin-3-yl)-carbamic acid tert-butyl ester

To an at 0° C. stirred solution of 105 mg (0.325 mmol) (R)-[5-(3-amino-phenyl)-5-fluoromethyl-5,6-dihydro-2H-[1,4]oxazin-3-yl]-carbamic acid tert-butyl ester, 72 mg (0.357 mmol) 5-bromo-pyridine-2-carboxylic acid, 57 mg (0.422 mmol) HOAt and 82 mg (0.812 mmol) Et₃N in 3 ml DCM were added 81 mg (0.422 mmol) EDC.HCl. After 18 h the mixture was diluted with EtOAc and washed with water, 5% aqueous NaHCO₃ and brine. Chromatography on silica gel (hexane/EtOAc 3:1) gave the desired product as a colorless solid.

TLC: Rf (Hexane/EtOAc 2:1)=0.31.

HPLC: Rt_{H4}=2.481 min; ESIMS [M+H]⁺=507/509(1Br);

¹H-NMR (360 MHz, CDCl3, major rotamer only): 9.80 (br s, 1H), 8.61 (s, 1H), 8.13 (d, 1H), 7.87 (dd, 1H), 7.77 (br s, 1H), 7.73 (d, 1H), 7.35 (br, 1H), 7.15 (t, 1H), 4.90-4.20 (m, 5H), 4.15 (d, 1H), 3.75 (br, 1H), 1.45 (br s, 9H).

### c) 5-Bromo-pyridine-2-carboxylic acid [3-((R)-5-amino-3-fluoromethyl-3,6-dihydro-2H-[1,4]oxazin-3-yl)-phenyl]-amide

A solution of 117 mg (0.231 mmol) ((R)-5-{3-[(5-bromo-pyridine-2-carbonyl)-amino]-phenyl}-5-fluoromethyl-5,6-dihydro-2H-[1,4]oxazin-3-yl)-carbamic acid tert-butyl ester in 2 ml 4N HCl in dioxane was stirred at 45° C. overnight. The mixture was concentrated and crystallized from EtOAc/hexane to yield the title compound as colorless crystals.

Rf (DCM/[MeOH/NH3 aqueous, 25%; 9:1:0.1)=0.15

HPLC: Rt_{H3}=2.786 min; ESIMS [M+H]⁺=407/409(1Br);

¹H-NMR (600 MHz, DMSO-d6): δ 10.81 (s, 1H), 10.78 (s, 1H), 8.88 (s, 1H), 8.65 (s, 1H), 8.36 (d, 1H), 8.10 (d, 1H), 8.00 (d, 1H), 7.48 (t, 1H), 7.28 (d, 1H), 4.89 (d, 2H, CH2F), 4.62 (s, 2H), 4.10 (d, 1H), 4.01 (d, 1H).

### Example 34

### 5-Bromo-pyridine-2-carboxylic acid [3-(5-amino-3-difluoromethyl-3,6-dihydro-2H-[1,4]oxazin-3-yl)-phenyl]-amide hydrochloride

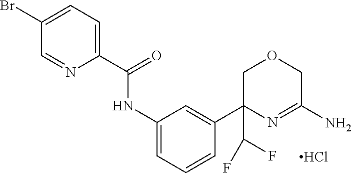 [SMILES: NC1=NC(c2cccc(NC(=O)c3ccc(Br)cn3)c2)(C(F)F)COC1 ; Cl]

### a) 1-(3-Bromo-phenyl)-2,2-difluoro-ethanone

1-Bromo-3-iodo-benzene (22.5 g, 90 mmol, Aldrich) was dissolved in THF and cooled to −78° C. nBuLi (69.8 ml, 90 mmol) was added over 15 minutes and the reaction was stirred for 30 min. at −78° C. Difluoro-acetic acid ethyl ester (16.59 ml, 153 mmol, Aldrich) was added dropwise and stirring was continued for 3 hrs. After completion the reaction was quenched by the addition of 329 ml 2 M HCl solution and reaction was warmed to r.t. The phases were separated and the aqueous phases was extracted with Et₂O. The organic phases were washed with water and brine, dried over Na₂SO₄ and concentrated under reduced pressure. The residue was purified by automated column chromatography (cyclohexane/ethyl acetate) to yield the title compound as a yellow oil. ¹H-NMR (360 MHz, DMSO-d₆): 8.18 (s, 1H), 8.02 (m, 2H), 7.61 (t, 1H), 7.21 (t, 1H, CHF₂); GC/MS: 234 [(M+H)⁺].

### b) [1-(3-Bromo-phenyl)-2,2-difluoro-ethylidene]-carbamic acid tert-butyl ester

1-(3-Bromo-phenyl)-2,2-difluoro-ethanone (15.36 g, 65.4 mmol) and N-boc-imino(triphenyl)phosphorane (27.1 g, 71.9 mmol) were heated for 75 hrs in toluene under N₂. After completion, volatiles were removed under reduced pressure and 457 ml hexane were added. The reaction was heated to reflux, cooled down and the formed precipitate was filtered off. The filtrate was evaporated yielding the crude product which was purified by column chromatography (cylcohexane/TBME). Yellow oil was obtained as product. ¹H-NMR (360 MHz, DMSO-d₆): 8.02 (m, 1H), 7.85 (m, 1H), 7.55 (m, 2H), 7.20 (t, 1H, CHF₂); MS: 234 [(M+H-Boc)⁺].

### c) [1-(3-Bromo-phenyl)-1-difluoromethyl-allyl]-carbamic acid tert-butyl ester

[1-(3-Bromo-phenyl)-2,2-difluoro-ethylidene]-carbamic acid tert-butyl ester (9.09 g, 27.2 mmol) was dissolved in toluene and cooled to −20° C. under N₂. Using a syringe pump, vinylmagnesiumbromide (42.5 ml, 34.0 mmol) was added (1 eq. per hour). After 1.25 hrs no starting material was left and 218 ml half-saturated NH₄Cl solution was added to the reaction. The product was extracted with TBME. The organic phases were washed with water and brine, dried over Na₂SO₄ and concentrated under reduced pressure. The residue was purified by automated column chromatography (cyclohexane/TBME) to yield the title compound as a yellowish oil. ¹H-NMR (600 MHz, DMSO-d₆): 7.81 (br, 1H, NH), 7.52 (m, 2H), 7.35 (m, 2H), 6.48 (t, 1H, CHF₂), 5.45 (d, 1H), 5.15 (d, 1H), 1.32 (s, 9H); MS: 362 [(M+H)⁺].

### d) [1-(3-Bromo-phenyl)-2,2-difluoro-1-hydroxymethyl-ethyl]-carbamic acid tert-butyl ester

[1-(3-Bromo-phenyl)-1-difluoromethyl-allyl]-carbamic acid tert-butyl ester (7.88 g, 21.76 mmol) was dissolved in 218 ml dichloromethane and 73 ml methanol. NaHCO3 (2.74 g, 32.6 mmol) was added and the reaction mixture was cooled to −78° C. The solution was treated with O₃ for 30 min. (until the reaction mixture turned blue). Gas was stopped and stirring was continued for 15 minutes. The reaction was flushed with oxygen and nitrogen until color disappeared. NaBH4 (2.47 g, 65.3 mmol) was added in three portions and stirring was continued for 30 min. at −78° C. The reaction was warmed to 0° C. and poured onto 435 ml 1 M HCl solution. The product was extracted with TBME. The organic phases were washed with water and brine, dried over Na₂SO₄ and concentrated under reduced pressure to yield the title compound as a greenish oil. ¹H-NMR (600 MHz, DMSO-d₆): 7.49 (m, 2H), 7.38 (br, 1H, NH), 7.32 (m, 2H), 6.37 (t, 1H, CHF₂), 5.20 (br, 1H), 3.95 (m, 1H), 3.86 (br, 1H), 1.32 (s, 9H); MS: 366 [(M+H)⁺].

### e) 2-Amino-2-(3-bromo-phenyl)-3,3-difluoro-propan-1-ol hydrochloride

[1-(3-Bromo-phenyl)-2,2-difluoro-1-hydroxymethyl-ethyl]-carbamic acid tert-butyl ester (8.408 g, 22.96 mmol) was dissolved in 105 ml 4 N HCl in dioxane. The reaction was stirred for 45 min. After completion volatiles were removed under reduced pressure to yield a white solid. ¹H-NMR (360 MHz, DMSO-d₆): 9.30 (br, 3H, NH₃⁺), 7.85 (s, 1H), 7.70 (d, 1H), 7.60 (d, 1H), 7.49 (t, 1H), 6.63 (t, 1H, CHF₂), 6.03 (br, 1H), 4.05 (m, 2H); MS: 266 [(M+H)⁺].

### f) N-[1-(3-Bromo-phenyl)-2,2-difluoro-1-hydroxymethyl-ethyl]-2-chloro-acetamide

2-Amino-2-(3-bromo-phenyl)-3,3-difluoro-propan-1-ol hydrochloride (6.5 g, 24.43 mmol) was put between 60 ml aqeous 2 M Na₂CO₃ solution and 60 ml dichloromethane and cooled to 0° C. under strong stirring. Then chloroacetylchloride (2.94 ml, 36.6 mmol), diluted in 8 ml dichloromethane, was added dropwise to the biphasic solution. After the complete addition, the reaction was stirred for 30 minutes at r.t. After completion 10 ml MeOH were added and stirring was continued for 10 minutes. Then TBME and water were added. The phases were separated, and the aqueous phase was extracted with TBME. The organic phases were washed with water and brine, dried over Na₂SO₄ and concentrated under reduced pressure to yield the title compound as off-white solid. ¹H-NMR (360 MHz, DMSO-d₆): 8.72 (s, 1H), 7.53 (m, 2H), 7.35 (m, 2H), 6.48 (t, 1H, CHF₂), 5.39 (t, 1H), 4.18 (m, 2H), 3.10 (s, 2H); MS: 342 [(M+H)⁺].

### g) 5-(3-Bromo-phenyl)-5-difluoromethyl-morpholin-3-one

N-[1-(3-Bromo-phenyl)-2,2-difluoro-1-hydroxymethyl-ethyl]-2-chloro-acetamide (8.10 g, 23.65 mmol) and potassium tert-butoxide (5.31 g, 47.3 mmol) were heated to 95° C. in 118 ml tert-butanol for 30 minutes. After completion water was added and the reaction was evaporated. The residue was put between ethyl acetate and water. The phases were separated and the aqueous phase was extracted twice with ethyl acetate. The organic phases were washed with water and brine, dried over Na₂SO₄ and concentrated under reduced pressure to yield the title compound as off-white solid. ¹H-NMR (360 MHz, DMSO-d₆): 9.13 (s, 1H, NH), 7.78 (s, 1H), 7.59 (m, 2H), 7.42 (t, 1H), 6.48 (t, 1H, CHF₂), 4.28 (d, 1H), 4.10 (m, 2H), 3.92 (m, 1H); MS: 306 [(M+H)⁺].

### h) 5-(3-Bromo-phenyl)-5-difluoromethyl-morpholine-3-thione

5-(3-Bromo-phenyl)-5-difluoromethyl-morpholin-3-one (6.10 g, 19.93 mmol) was dissolved in 63 ml dry pyridine, and P₂S₅ (5.32 g, 23.91 mmol) was added. The mixture was heated to 80° V for 2 hrs. After completion, the mixture was put between ethyl acetate and 1 H HCl solution. Phases were separated and the organic phase was washed with 1 N HCl, saturated NaHCO₃ solution and brine. The organic phases were combined, dried over Na₂SO₄ and concentrated under reduced pressure to yield the title compound as off-white solid. ¹H-NMR (360 MHz, DMSO-d₆): 8.60 (s, 1H, NH), 7.80-7.35 (m, 4H), 6.54 (t, 1H, CHF₂), 4.45 (m, 2H), 4.28 (d, 1H), 4.12 (d, 1H); MS: 322 [(M+H)⁺].

### i) 5-(3-Bromo-phenyl)-5-difluoromethyl-5,6-dihydro-2H-[1,4]oxazin-3-ylamine

5-(3-Bromo-phenyl)-5-difluoromethyl-morpholine-3-thione (7.49 g, 23.25 mmol) was dissolved in 36 ml 7N NH₃ in methanol and stirred at room temperature for 18 h. After completion, volatiles were removed under reduced pressure to yield the title compound. ¹H-NMR (360 MHz, DMSO-d₆): 7.80 (s, 1H), 7.52 (m, 2H), 7.33 (m, 1H), 6.25 (br, 2H, NH₂), 6.04 (t, 1H, CHF₂), 4.15-3.90 (m, 2H), 3.72 (d, 1H), 3.45 (d, 1H); MS: 305 [(M+H)⁺].

### j) [5-(3-Bromo-phenyl)-5-difluoromethyl-5,6-dihydro-2H-[1,4]oxazin-3-yl]-carbamic acid tert-butyl ester

5-(3-Bromo-phenyl)-5-difluoromethyl-5,6-dihydro-2H-[1,4]oxazin-3-ylamine (6.12 g, 20.06 mmol) was dissolved in 100 ml ACN under N₂ at 0° C. Then Boc₂O (5.69 g, 26.1 mmol), DIPEA (5.25 ml, 30.1 mmol) and DMAP (0.25 g, 2.01 mmol) were added and the ice bath was removed. The reaction was stirred at room temperature for 90 min. After completion, the reaction was diluted with water and extracted with dichloromethane. The organic phases were washed with 1N HCl, saturated NaHCO₃ sol., water and brine, dried over Na₂SO₄ and concentrated under reduced pressure. The residue was purified by automated column chromatography (cyclohexane/TBME) to yield the title compound as a brownish solid. ¹H-NMR (360 MHz, DMSO-d₆): 9.97 (s, 1H, NH), 7.80 (s, 1H), 7.55 (m, 2H), 7.35 (m, 1H), 6.17 (t, 1H, CHF₂), 4.62 (d, 1H), 4.41 (d, 1H), 4.22 (d, 1H), 3.75 (d, 1H), 1.45 (s, 9H); MS: 405 [(M+H)⁺].

### k) [5-(3-Azido-phenyl)-5-difluoromethyl-5,6-dihydro-2H-[1,4]oxazin-3-yl]-carbamic acid tert-butyl ester

[5-(3-Bromo-phenyl)-5-difluoromethyl-5,6-dihydro-2H-[1,4]oxazin-3-yl]-carbamic acid tert-butyl ester (2.25, 5.55 mmol), sodium azide (2.89 g, 44.4 mmol), sodium ascorbate (0.440 g, 2.22 mmol), copper iodide (0.423 g, 2.22 mmol) and rac-trans-N,N′-dimethyl-cyclohexane-1,2-diamine (0.790 g, 5.55 mmol) were dissolved in ethanol (76.6 ml) and water (33.0 ml). The mixture was stirred under N₂ at 70° C. for 30 minutes. After completion hexane/ethyl acetate 1/1 were added and the reaction mixture was filtered over silica gel. The filtrate was evaporated and the residue was purified by chromatography on silica gel (cyclohexane/TBME 9/1 to obtain azide, later hexane/ethyl acetate 2/1 to 1/1 to obtain aniline as side product) to yield the title compound. ¹H-NMR (360 MHz, CDCl₃): 7.42 (m, 1H), 7.25 (m, 2H), 7.07 (m, 1H), 5.97 (t, 1H, CHF₂), 4.80 (d, 1H), 4.65 (d, 1H), 4.25 (d, 1H), 3.80 (d, 1H), 1.55 (s, 9H); MS: 368 [(M+H)⁺].

### l) [5-(3-Amino-phenyl)-5-difluoromethyl-5,6-dihydro-2H-[1,4]oxazin-3-yl]-carbamic acid tert-butyl ester

A solution of [5-(3-azido-phenyl)-5-difluoromethyl-5,6-dihydro-2H-[1,4]oxazin-3-yl]-carbamic acid tert-butyl ester (1.71 g, 4.65 mmol) in 22 ml ethanol and 12 ml THF was hydrogenated with Pd/C (10%) (3 hrs, r.t.) The mixture was filtered through Celite, and the filtrate was evaporated and the residue was purified by chromatography on silica gel (cyclohexane/ethyl acetate) to yield the title compound as colorless solid. ¹H-NMR (360 MHz, CDCl₃): 7.22 (m, 1H), 6.92 (m, 1H), 6.84 (m, 1H), 6.70 (m, 1H), 5.95 (t, 1H, CHF₂), 4.92 (m, 1H), 4.73 (m, 1H), 4.32 (m, 1H), 3.95 (m, 1H), 1.53 (s, 9H); MS: 342 [(M+H)⁺].

### m) (5-{3-[(5-Bromo-pyridine-2-carbonyl)-amino]-phenyl}-5-difluoromethyl-5,6-dihydro-2H-[1,4]oxazin-3-yl)-carbamic acid tert-butyl ester

[5-(3-Amino-phenyl)-5-difluoromethyl-5,6-dihydro-2H-[1,4]oxazin-3-yl]-carbamic acid tert-butyl ester and 5-bromo-pyridine-2-carboxylic acid were coupled according the procedure described in example 51m). ¹H-NMR (360 MHz, CDCl₃): 9.92 (s, 1H, NH), 8.73 (s, 1H), 8.24 (d, 1H), 8.10 (d, 1H), 7.88 (m, 2H), 7.45 (m, 1H), 7.31 (m, 1H), 5.92 (t, 1H, CHF₂), 4.84 (d, 1H), 4.65 (d, 1H), 4.31 (d, 1H), 3.96 (d, 1H), 1.52 (s, 9H); MS: 525 [(M+H)⁺].

### n) 5-Bromo-pyridine-2-carboxylic acid [3-(5-amino-3-difluoromethyl-3,6-dihydro-2H-[1,4]oxazin-3-yl)-phenyl]-amide hydrochloride

(5-{3-[(5-Bromo-pyridine-2-carbonyl)-amino]-phenyl}-5-difluoromethyl-5,6-dihydro-2H-[1,4]oxazin-3-yl)-carbamic acid tert-butyl ester was deprotected according to the procedure described in example 51). ¹H-NMR (500 MHz, DMSO-d₆): 10.85 (s, 1H), 10.25 (s, 1H), 9.65 (br, 1H), 8.90 (s, 1H), 8.67 (br, 1H), 8.35 (m, 1H), 8.09 (d, 1H), 7.99 (m, 2H), 7.50 (t, 1H), 7.30 (d, 1H), 6.70 (t, 1H, CHF₂), 4.63 (m, 2H), 4.38 (m, 1H), 4.05 (m, 1H); MS: 425 [(M+H)⁺].

### Examples 35 to 41

The compounds listed in Table 3 were prepared by a procedure analogous to thoat used in example 34.

TABLE 3

| | | | MS |
|---|---|---|---|
| | | ¹H-NMR | [m/z; |
| Example | Compound | (δ; DMSO-d₆) | (M + 1)⁺] |
| 35 | 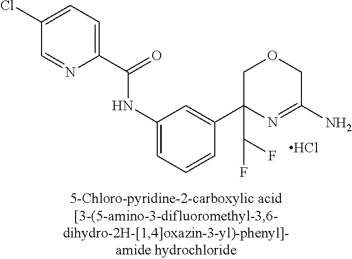 [SMILES: NC1=NC(c2cccc(NC(=O)c3ccc(Cl)cn3)c2)(C(F)F)COC1 ; Cl] | 10.78 (s, 1H), 9.71 (s, 1H), 8.82 (s, 2H), 8.22 (m, 2H), 8.04 (m, 2H), 7.53 (t, 1H), 7.35 (d, 1H), 6.74 (t, 1H, CHF₂), 4.67 (m, 2H), 4.41 (m, 1H), 4.08 (m, 1H) | 381 |
| 36 | 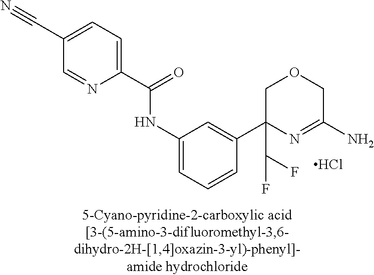 [SMILES: N#Cc1ccc(C(=O)Nc2cccc(C3(C(F)F)COCC(N)=N3)c2)nc1 ; Cl] | 10.93 (s, 1H), 9.73 (s, 1H), 9.24 (s, 1H), 8.86 (s, 1H), 8.64 (d, 1H), 8.32 (d, 1H), 8.05 (m, 2H), 7.54 (t, 1H), 7.37 (d, 1H), 6.73 (t, 1H, CHF₂), 4.68 (m, 2H), 4.43 (m, 1H), 4.07 (m, 1H) | 372 |
| 37 | 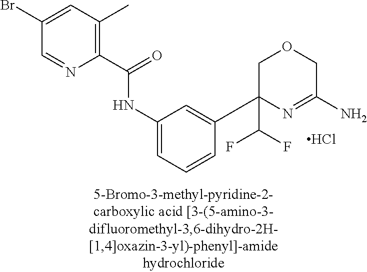 [SMILES: Cc1cc(Br)cnc1C(=O)Nc1cccc(C2(C(F)F)COCC(N)=N2)c1 ; Cl] | 11.13 (s, 1H), 10.75 (s, 1H), 9.73 (s, 1H), 8.91 (s, 1H), 8.68 (s, 1H), 8.21 (s, 1H), 7.92 (m, 2H), 7.48 (t, 1H), 7.32 (d, 1H), 6.71 (t, 1H, CHF₂), 4.65 (m, 2H), 4.40 (m, 1H), 4.05 (m, 1H), 2.58 (s, 3H) | 439 |
| 38 | 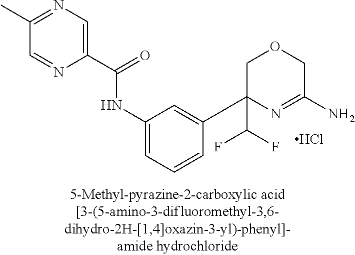 [SMILES: Cc1cnc(C(=O)Nc2cccc(C3(C(F)F)COCC(N)=N3)c2)cn1 ; Cl] | 11.13 (s, 1H), 10.80 (s, 1H), 9.71 (s, 1H), 9.16 (s, 1H), 8.89 (s, 1H), 8.72 (s, 1H), 8.05 (s, 1H), 7.99 (d, 1H), 7.30 (t, 1H), 7.35 (d, 1H), 6.71 (t, 1H, CHF₂), 4.65 (m, 2H), 4.40 (m, 1H), 4.05 (m, 1H), 2.58 (s, 3H) | 362 |
| 39 | 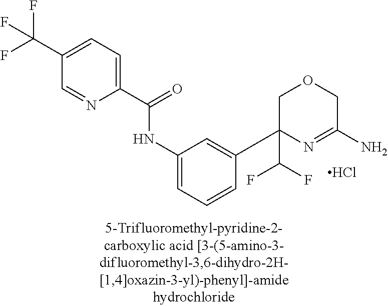 [SMILES: NC1=NC(c2cccc(NC(=O)c3ccc(C(F)(F)F)cn3)c2)(C(F)F)COC1 ; Cl] | 11.1 (s, 1H), 10.94 (s, 1H), 9.70 (s, 1H), 8.82 (s, 1H), 8.53 (d, 1H), 8.36 (d, 1H), 8.04 (m, 2H), 7.53 (t, 1H), 7.35 (d, 1H), 6.72 (t, 1H, CHF₂), 4.68 (m, 2H), 4.41 (m, 1H), 4.08 (m, 1H) | 415 |
| 40 | 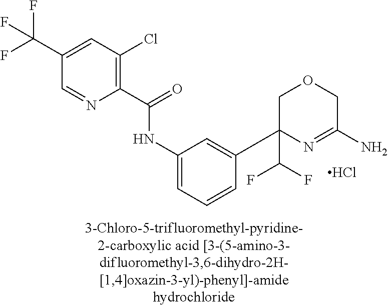 [SMILES: NC1=NC(c2cccc(NC(=O)c3ncc(C(F)(F)F)cc3Cl)c2)(C(F)F)COC1 ; Cl] | 11.1 (s, 1H), 11.02 (s, 1H), 9.70 (s, 1H), 9.10 (s, 1H), 8.77 (s, 1H), 8.73 (s, 1H), 7.87 (s, 1H), 7.78 (d, 1H), 7.53 (t, 1H), 7.36 (d, 1H), 6.73 (t, 1H, CHF₂), 4.68 (m, 2H), 4.41 (m, 1H), 4.10 (m, 1H) | 449 |
| 41 | 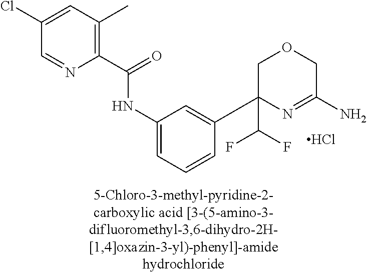 [SMILES: Cc1cc(Cl)cnc1C(=O)Nc1cccc(C2(C(F)F)COCC(N)=N2)c1 ; Cl] | 11.18 (s, 1H), 10.68 (s, 1H), 9.75 (s, 1H), 8.95 (s, 1H), 8.59 (s, 1H), 8.06 (s, 1H), 7.92 (m, 2H), 7.49 (t, 1H), 7.34 (d, 1H), 6.71 (t, 1H, CHF₂), 4.67 (m, 2H), 4.40 (m, 1H), 4.05 (m, 1H), 2.58 (s, 3H) | 395 |

### Example 42

### 5-Bromo-pyridine-2-carboxylic acid [3-(5-amino-3-difluoromethyl-3,6-dihydro-2H-[1,4]oxazin-3-yl)-4-fluoro-phenyl]-amide hydrochloride

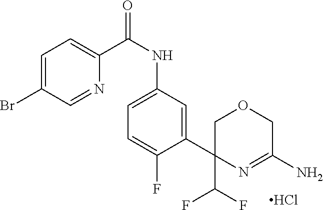 [SMILES: NC1=NC(c2cc(NC(=O)c3ccc(Br)cn3)ccc2F)(C(F)F)COC1 ; Cl]

### a) 1-(5-Bromo-2-fluoro-phenyl)-ethanone

A solution of 17.78 ml (126 mmol) diisopropyl amine in 375 ml THF was cooled to −78° C. A 1.6 M solution of BuLi in hexanes (79 ml, 126 mmol) was added drop wise. After 15 minutes 20 g of 4-bromo-1-fluoro benzene (114 mmol) was added dropwise while keeping the temperature below −60° C. After stirring for 2.5 h at −70° C. 13.22 ml ethyl difluoro acetate were added. The mixture was warmed to −40° C. and then quenched by pouring the mixture onto 1M HCl. The mixture was extracted with ligroine, dried with MgSO₄.H₂O, concentrated and purified by column chromatography (silica gel; hexane/5-15% TBME) to give the desired product as a yellow liquid. ¹H-NMR (CDCl₃, 360 MHz): 8.09 (dd, 1H), 7.82-7.77 (m, 1H), 7.17 (t, 1H), 6.45 (t, 1H, CHF₂)

### b) 1-(5-Bromo-2-fluoro-phenyl)-1-difluoromethyl-allyl]-carbamic acid tert-butyl ester

A mixture of 16 g (63.2 mmol) 1-(5-bromo-2-fluoro-phenyl)-ethanone and 26.3 g (69.6 mmol) N-tert-butyloxycarbonyl-triphenyliminophosphorane were heated at 90° C. in toluene for 18 h. The mixture was triturated with hexane and filtered to remove triphenyl phosphine oxide. The filtrate was purified by chromatography on silica gel (hexane/1-5% TBME) to give 11.37 g (32.3 mmol) of the desired product as a slightly impure yellow oil.

TLC: Rf (Hexane/EtOAc 6:1)=0.65.

The product was dissolved in 100 ml THF and cooled to −78° C. Vinylmagnesium bromide (48 ml of a 1M solution in THF) was added dropwise, while the reaction temperature was not allowed to exceed −60° C. The mixture was stirred at −70° C. for 1 h before it was allowed to warm to 0° C. The reaction was quenched with 10% aqueous ammonium chloride and extracted with TBME. The organic layer was washed with brine, treated with activated charcoal and MgSO4.H2O and filtered over celite. The filtrated was concentrated and crystallized from hexane to give the desired product as colorless crystals.

HPLC: Rt_{H1}=3.575 min; ESIMS [M+Na]⁺=402/404(1Br);

¹H-NMR (CDCl₃, 360 MHz): 7.57 (dd, 1H), 7.51-7.45 (m, 1H), 7.00 (dd, 1H), 6.49 (t, 1H, CHF₂), 6.21 (dd, 1H), 5.59 (d, 1H), 5.40 (dd, 1H), 5.25 (br, 1H), 1.40 (br s, 9H).

### c) [1-(5-Bromo-2-fluoro-phenyl)-2,2-difluoro-1-hydroxymethyl-ethyl]-carbamic acid tert-butyl ester

A suspension of 10.99 g (28.9 mmol) 1-(5-bromo-2-fluoro-phenyl)-1-difluoromethyl-allyl]-carbamic acid tert-butyl ester and 3.84 g (43.4 mmol) sodium hydrogen carbonate in 200 ml DCM and 80 ml MeOH was cooled to −78° C. A mixture of O₃ in oxygen gas was introduced till the blue color persisted. The excess ozone was removed by bubbling through oxygen gas for 10 minutes. NaBH4 (2.187 g, 57.8 mmol) was added as a solid in three portions. The mixture was stirred 10 min at −78° C. and then allowed to warm to 0° C. After 30 min the mixture was poured onto ice-cold 1N HCl and extracted with TBME. The organic phase was washed with 1N HCl, brine, dried with MgSO4.H2O and evaporated. The crude product was crystallized from hexane to give the desired product as colorless crystals.

TLC: Rf (Hexane/EtOAc 4:1)=0.29.

HPLC: Rt_{H1}=3.000 min; ESIMS [M+Na]⁺=406/408(1Br);

¹H-NMR (DMSO-d6, 360 MHz): 7.60-7.49 (m, 2H), 7.42 (br s, 1H), 7.180 (dd, 1H), 6.49 (t, 1H, CHF₂), 5.27 (br s, 1H), 3.90 (br s, 2H), 1.35 (br s, 9H).

### d) N-[1-(5-Bromo-2-fluoro-phenyl)-2,2-difluoro-1-hydroxymethyl-ethyl]-2-chloro-acetamide

A suspension of 10.22 g (26.6 mmol) [1-(5-bromo-2-fluoro-phenyl)-2,2-difluoro-1-hydroxymethyl-ethyl]-carbamic acid tert-butyl ester in 133 ml 4N HCl in dioxane was stirred for two h at rt. The mixture was evaporated to give the hydrochloride salt of 2-amino-2-(5-bromo-2-fluoro-phenyl)-3,3-difluoro-propan-1-ol.

HPLC: Rt_{H3}=2.550 min; ESIMS [M+H]⁺=284,286(1Br);

The crude product was taken up in 63 ml DCM and 63 ml 10% aqueous soda and stirred vigorously with ice-cooling. A solution of 3.34 ml (42 mmol) chloroacetyl chloride in 10 ml DCM was added dropwise. The ice bath was taken away and stirring was continued for 1 h. The mixture was diluted with TBME and water. The organic phase was dried with MgSO4.H2O and purified via chromatography on silica gel (hexane/25-33% EtOAc) to give the desired product as a slightly impure resin.

HPLC: Rt_{H3}=3.336 min; ESIMS [M+H]⁺=360/362/364 (1Br, 1Cl);

¹H-NMR (DMSO-d6, 360 MHz): 8.78 (s, 1H), 7.62-7.53 (m, 2H), 7.19 (dd, 1H), 6.53 (t, 1H, CHF₂), 5.43 (t, 1H), 4.27-4.02 (m, 4H).

### e) 5-(5-Bromo-2-fluoro-phenyl)-5-difluoromethyl-morpholin-3-one

A solution of 9.59 g (26.2 mmol) N-[1-(5-bromo-2-fluoro-phenyl)-2,2-difluoro-1-hydroxymethyl-ethyl]-2-chloro-acetamide in 134 ml t-butanol was treated with 3.58 g KOtBu. The mixture was heated at reflux for 3 h. After cooling down the mixture was diluted with EtOAc and 1N HCl. The organic phase was washed with brine, dried with MgSO4.H2O, filtered and evaporated. The product was obtained as colorless crystal (TBME/hexane).

TLC: Rf (Hexane/EtOAc 2:1)=0.29.

HPLC: Rt_{H3}=2.950 min; ESIMS [M+H]⁺=324/326(1Br);

¹H-NMR (CDCl3, 360 MHz): 7.61-7.55 (m, 2H), 7.09 (dd, 1H), 6.80 (br, 1H), 6.35 (t, 1H, CHF2), 4.37-4.17 (m, 4H).

### f) 5-(5-Bromo-2-fluoro-phenyl)-5-difluoromethyl-morpholine-3-thione

A mixture of 7.34 g (22.65 mmol) 5-(5-bromo-2-fluoro-phenyl)-5-difluoromethyl-morpholin-3-one and 5.19 g (12.46 mmol) Lawesson's reagent in 73 ml of THF was refluxed for 1 h. The mixture was concentrated and crystallized from DCM/hexane and recrystallized from EtOH to yield the desired product as colorless crystals.

HPLC: Rt_{H3}=3.370 min; ESIMS [M+H]⁺=340/342(1Br);

¹H-NMR (DMSO-d6, 360 MHz): 11.40 (s, 1H), 7.77-7.70 (m, 1H), 7.63 (dd, 1H), 7.37 (dd, 1H), 6.35 (t, 1H, CHF2), 4.50 (d, 1H), 4.44 (d, 1H), 4.29 (d, 1H), 4.10 (d, 1H).

### g) 5-(5-Bromo-2-fluoro-phenyl)-5-difluoromethyl-5,6-dihydro-2H-[1,4]oxazin-3-ylamine

A solution of 6.14 g (18.05 mmol) 5-(5-bromo-2-fluoro-phenyl)-5-difluoromethyl-morpholine-3-thione in 77 ml 7M NH3/MeOH was stirred at rt for 6 h. The mixture was evaporated and purified chromatographed on silica gel (DCM/1-5% MeOH followed by DCM/MeOH/aqueous NH3 95:4.5:0.5) to give the desired product as yellowish resin.

HPLC: Rt_{H3}=2.477 min; ESIMS [M+H]⁺=323/325(1Br);

¹H-NMR (DMSO-d6, 360 MHz): 7.99 (dd, 1H), 7.62-7.56 (m, 1H), 7.22 (dd, 1H), 6.31 (br, 2H), 6.12 (t, 1H, CHF2), 4.25 (d, 1H), 4.05 (d, 1H), 3.94 (d, 1H), 3.75 (d, 1H).

### h) [5-(5-Bromo-2-fluoro-phenyl)-5-difluoromethyl-5,6-dihydro-2H-[1,4]oxazin-3-yl]-carbamic acid tert-butyl ester

To an ice-cold solution of 6.38 g (19.75 mmol) 5-(5-bromo-2-fluoro-phenyl)-5-difluoromethyl-5,6-dihydro-2H-[1,4]oxazin-3-ylamine in 100 ml THF were added 5.60 g (25.67 mmol) Boc2O and 5.17 ml (29.6 mmol) DIPEA. The mixture was stirred for 4 h at rt. Then the mixture was diluted with TBME and washed with 5% aqueous NaHCO3. The organic phase was dried with MgSO4.H2O, filtered and concentrated. Purification by chromatography on silica gel (hexane/5-20% EtOAc) gave the desired product as a colorless solid.

TLC: Rf (Hexane/EtOAc 9:1)=0.27.

HPLC: Rt_{H1}=3.299 min; ESIMS [M+H]⁺=423/425(1Br);

¹H-NMR (CDCl3, 360 MHz): 7.81 (dd, 1H), 7.50-7.44 (m, 1H), 7.00 (dd, 1H), 6.12 (t, 1H, CHF2), 4.83 (d, 1H), 4.60 (d, 1H), 4.37 (dd, 1H), 3.94 (d, 1H), 1.52 (s, 9H).

### i) [5-(5-Azido-2-fluoro-phenyl)-5-difluoromethyl-5,6-dihydro-2H-[1,4]oxazin-3-yl]-carbamic acid tert-butyl ester

To a solution of 7.27 g (17.18 mmol) [5-(5-bromo-2-fluoro-phenyl)-5-difluoromethyl-5,6-dihydro-2H-[1,4]oxazin-3-yl]-carbamic acid tert-butyl ester and 2.443 g (17.18 mmol) trans-N,N′-dimethylcyclohexane-1,2-diamine in 237 ml EtOH was added a solution of 8.93 g (137 mmol) sodium azide and 1.361 g (6.87 mmol) sodium-ascorbate in 102 ml water. The mixture was degassed and brought under nitrogen atmosphere. CuI (1.309 g, 6.87 mmol) was added and the mixture was heated at 70° C. The initially formed suspension turned into a homogeneous blue solution. The mixture was cooled to rt, diluted with water and TBME. The organic phase was washed with brine and dried with MgSO4.H2O. The crude product was purified by chromatography on silica gel (hexane/5-8% TBME) to give the desired product as a colorless solid.

HPLC: Rt_{H1}=3.173 min; ESIMS [M+H]⁺=386;

¹H-NMR (CDCl3, 360 MHz, signals broadened due to rotamers): 7.39-7.44 (m, 1H), 7.15-7.06 (m, 1H), 7.05-6.98 (m, 1H), 6.14 (t, 1H, CHF2), 4.80 (d, 1H), 4.60 (d, 1H), 4.39 (d, 1H), 3.97 (d, 1H), 1.52 (s, 9H).

### j) [5-(5-Amino-2-fluoro-phenyl)-5-difluoromethyl-5,6-dihydro-2H-[1,4]oxazin-3-yl]-carbamic acid tert-butyl ester

A solution of 4.89 g (12.69 mmol) [5-(5-azido-2-fluoro-phenyl)-5-difluoromethyl-5,6-dihydro-2H-[1,4]oxazin-3-yl]-carbamic acid tert-butyl ester in 64 ml EtOH and 17 ml THF was treated with 1.1 g 10% Pd—C and stirred under an atmosphere of hydrogen until the starting material had been consumed. The mixture was diluted with DCM and filtered over celite. The product was purified by chromatography on silica gel (hexane/25-50% EtOAc) to give the desired product as colorless foam.

HPLC: Rt_{H3}=2.778 min; ESIMS [M+H]⁺=360;

¹H-NMR (CDCl3, 360 MHz, spectrum not interpretable due to rotamers): 7.1-6.1 (m, ˜4H), 5.0-4.9 (m, 4H), 1.52 (br s, 9H).

### k) (5-{5-[(5-Bromo-pyridine-2-carbonyl)-amino]-2-fluoro-phenyl}-5-difluoromethyl-5,6-dihydro-2H-[1,4]oxazin-3-yl)-carbamic acid tert-butyl ester

To a solution of 325 mg (0.952 mmol) [5-(5-amino-2-fluoro-phenyl)-5-difluoromethyl-5,6-dihydro-2H-[1,4]oxazin-3-yl]-carbamic acid tert-butyl ester, 212 mg (1.047 mmol) 5-bromo-pyridine-2-carboxylic acid, 168 mg (1.238 mmol) HOAt and 0.34 ml (2.38 mmol) Et₃N in 5 ml DCM were added 237 mg (1.24 mmol) EDC.HCl. The mixture was stirred overnight. The mixture was diluted with EtOAc and washed with water, 1N HCl, brine and 5% aqueous NaHCO₃. The organic phase was dried with MgSO₄.H2O, filtered and purified by chromatography on silica gel (hexane/14-18% EtOAc) to give the desired product as colorless foam.

TLC: Rf (Hexane/EtOAc 3:1)=0.35.

HPLC: Rt_{H1}=3.127 min; ESIMS [M+H]⁺=525/527(1Br)

¹H-NMR (CDCl3, 360 MHz): 9.90 (br s, 1H), 8.72 (d, 1H), 8.23 (d, 1H), 8.09 (dd, 1H), 7.94-7.86 (m, 2H), 7.47 (t, 1H), 7.38-7.28 (m, 3H), 5.92 (t, 1H, CHF2), 4.87 (d, 1H), 4.67 (d, 1H), 4.6-4.45 (br, 1H), 4.34 (d, 1H), 4.00 (d, 1H), 1.56 (br s, 9H).

### l) 5-Bromo-pyridine-2-carboxylic acid [3-(5-amino-3-difluoromethyl-3,6-dihydro-2H-[1,4]oxazin-3-yl)-4-fluoro-phenyl]-amide

A solution of 100 mg (0.184 mmol) (5-{5-[(5-bromo-pyridine-2-carbonyl)-amino]-2-fluoro-phenyl}-5-difluoromethyl-5,6-dihydro-2H-[1,4]oxazin-3-yl)-carbamic acid tert-butyl ester in 1.4 ml 4N HCl in dioxane was stirred at 50° C. After 15 min 0.3 ml 3N HCl in MeOH were added and the now homogeneous solution was stirred for 3 h. The mixture was concentrated and crystallized from EtOH/TBME to yield the title compound.

HPLC: Rt_{H3}=2.857 min; ESIMS [M+H]⁺=443/445;

¹H-NMR (600 MHz, DMSO-d6): δ 10.93 (s, 1H), 9.78 (s, 1H), 8.89 (s, 1H), 8.77 (s, 1H), 8.35 (d, 1H), 8.11-8-06 (m, 3H), 7.39 (t, 1H), 6.79 (t, 1H, CHF2), 4.70 (d, 1H), 4.64 (d, 1H), 4.34 (d, 1H), 4.17 (d, 1H).

### Examples 43 to 49

The compounds listed in Table 4 were prepared by a procedure analogous to that used in example 42.

For enantiomerically pure compounds the racemic precursor [5-(5-amino-2-fluoro-phenyl)-5-difluoromethyl-5,6-dihydro-2H-[1,4]oxazin-3-yl]-carbamic acid tert-butyl ester (example 42j)) was separated via prep-HPLC on Chiralpak AD-H 250×4.6 mm column using supercritical CO2/EtOH 9:1 as an eluent. The desired compound was the slower eluting (R)-enantiomer. Enantiomeric excess=99.7%; [α]_{D}=−109.7° (c=1, CHCl₃).

TABLE 4

| | | | MS |
|---|---|---|---|
| | | ¹H-NMR | [m/z; |
| Example | Compound | (δ; DMSO-d₆) | (M + 1)⁺] |
| 43 | 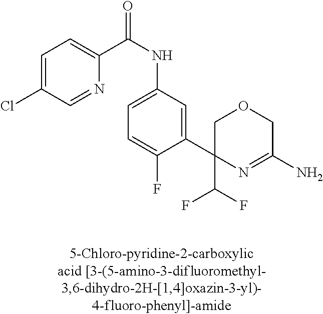 [SMILES: NC1=NC(c2cc(NC(=O)c3ccc(Cl)cn3)ccc2F)(C(F)F)COC1] | 10.95 (s, 1H), 9.77 (s, 1H), 8.80 (s and s, 2H), 8.22 (dd, 1H), 8.17 (d, 1H), 8.10-8.06 (m, 2H), 7.39 (t, 1H), 6.78 (t, 1H, CHF₂), 4.72 (d, 1H), 4.65 (d, 1H), 4.32 (d, 1H), 4.18 (d, 1H). | 399 |
| 44 | 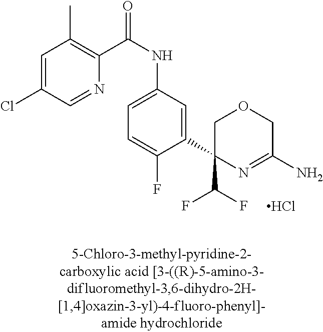 [SMILES: Cc1cc(Cl)cnc1C(=O)Nc1ccc(F)c([C@]2(C(F)F)COCC(N)=N2)c1 ; Cl] | 10.78 (s, 1H), 9.78 (s, 1H), 8.78 (s, 1H), 8.62 (s, 1H), 8.01-7.94 (m, 2H), 7.39 (dd, 1H), 6.78 (t, 1H, CHF2), 4.72 (d, 1H), 4.68 (d, 1H), 4.39 (d, 1H), 4.21 (d, 1H). | 413 |
| 45 | 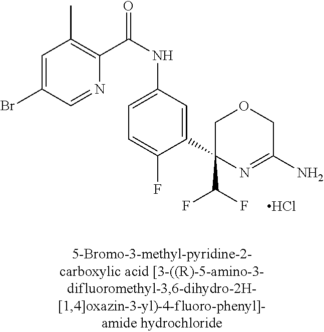 [SMILES: Cc1cc(Br)cnc1C(=O)Nc1ccc(F)c([C@]2(C(F)F)COCC(N)=N2)c1 ; Cl] | 10.64 (s, 1H), 9.78 (s, 1H), 8.67 (s, 1H), 8.19 (s, 1H), 8.01-7.94 (m, 2H), 7.38 (dd, 1H), 6.78 (t, 1H, CHF2), 4.75 (d, 1H), 4.65 (d, 1H), 4.34 (d, 1H), 4.17 (d, 1H). | 457 |
| 46 | 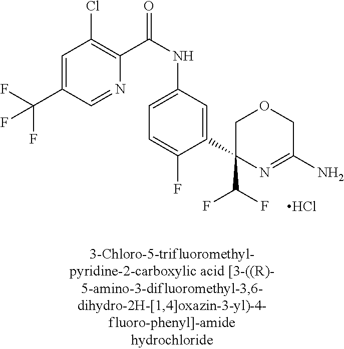 [SMILES: NC1=N[C@@](c2cc(NC(=O)c3ncc(C(F)(F)F)cc3Cl)ccc2F)(C(F)F)COC1 ; Cl] | 11.05 (s, 1H), 9.74 (s, 1H), 9.08 (s, 1H), 8.80 (s, 1H), 8.74 (s, 1H), 8.90-8.84 (m, 2H), 7.41 (dd, 1H), 6.78 (t, 1H, CHF2), 4.69 (d, 1H), 4.64 (d, 1H), 4.35 (d, 1H), 4.17 (d, 1H). | 467 |
| 47 | 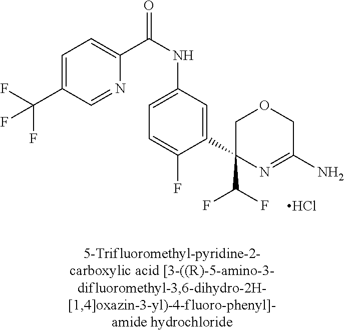 [SMILES: NC1=N[C@@](c2cc(NC(=O)c3ccc(C(F)(F)F)cn3)ccc2F)(C(F)F)COC1 ; Cl] | 11.02 (s, 1H), 9.75 (s, 1H), 9.15 (s, 1H), 8.74 (s, 1H), 8.52 (d, 1H), 8.34 (d, 1H), 8.12-8.06 (m, 2H), 7.40 (dd, 1H), 6.78 (t, 1H, CHF2), 4.70 (d, 1H), 4.60 (d, 1H), 4.31 (d, 1H), 4.18 (d, 1H). | 433 |
| 48 | 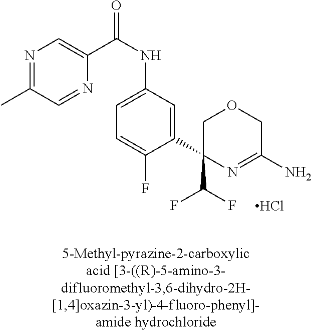 [SMILES: Cc1cnc(C(=O)Nc2ccc(F)c([C@]3(C(F)F)COCC(N)=N3)c2)cn1 ; Cl] | 11.00 (s, 1H), 10.95 (s, 1H), 9.73 (s, 1H), 9.17 (s, 1H), 8.73 (d and d, 2H), 8.14-8.10 (m, 1H), 8.08-8.03 (m, 1H), 7.40 (dd, 1H), 6.78 (t, 1H, CHF2), 4.70 (d, 1H), 4.65 (d, 1H), 4.32 (d, 1H), 4.18 (d, 1H), 2.56 (s, 3H). | 380 |
| 49 | 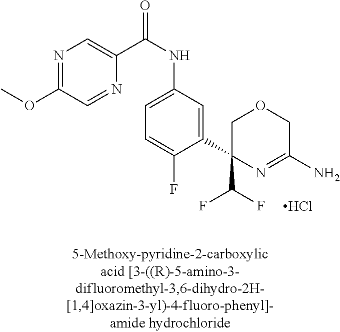 [SMILES: COc1cnc(C(=O)Nc2ccc(F)c([C@]3(C(F)F)COCC(N)=N3)c2)cn1 ; Cl] | 10.99 (s, 1H), 10.74 (s, 1H), 9.75 (s, 1H), 8.71 (s, 1H), 8.39 (s, 1H), 8.15 (d, 1H), 8.10-8.14 (m, 2H), 7.40 (dd, 1H), 7.64 (d, 1H), 6.37 (dd, 1H), 6.78 (t, 1H, CHF2), 4.72 (d, 1H), 4.63 (d, 1H), 4.33 (d, 1H), 4.17 (d, 1H), 3.44 (s, 3H). | 395 |

### Example 50

### 5-Chloro-pyridine-2-carboxylic acid [3-(5-amino-3-trifluoromethyl-3,6-dihydro-2H-[1,4]oxazin-3-yl)-4-fluoro-phenyl]-amide

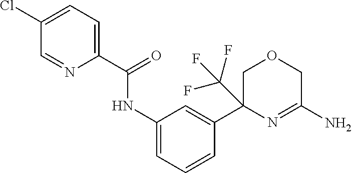 [SMILES: NC1=NC(c2cccc(NC(=O)c3ccc(Cl)cn3)c2)(C(F)(F)F)COC1]

### a) 2-(3-Bromo-phenyl)-2-tert-butoxycarbonylamino-3,3,3-trifluoro-propionic acid ethyl ester

A solution of 5.66 g (20 mmol) 1-bromo-3-iodo-benzene in 25 ml THF was stirred at −20° C. A 1.82 M solution of isopropylmagnesium chloride (12.1 ml, 22.0 mmol) in THF was added and the mixture was stirred 1 h at 0° C. The mixture was cooled to −78° C. and a solution of 5.38 g (20 mmol) 2-[(E)-tert-butoxycarbonylimino]-3,3,3-trifluoro-propionic acid ethyl ester in 50 ml of THF was added over a period of 2 h. After 20 min the mixture was quenched with 5% aqueous NH₄Cl. The mixture was extracted with TBME. The organic phase was dried with Na2SO4, filtered and evaporated. Purification via chromatography on silica gel (c-hexane/0-14% EtOAc) gave the desired product as a colorless resin.

TLC: Rf (Hexane/EtOAc 6:1)=0.37.

HPLC: Rt_{H1}=3.704 min; ESIMS [M+Na]⁺=448/450(1Br);

¹H-NMR (CDCl3, 360 MHz): 7.69 (s, 1H), 7.57 (d, 1H), 7.49 (d, 1H), 7.31 (t, 1H), 5.70 (br s, 1H), 4.37 (q, 2H), 1.42 (br s, 9H), 1.30 (t, 3H).

### b) [1-(3-Bromo-phenyl)-2,2,2-trifluoro-1-hydroxymethyl-ethyl]-carbamic acid tert-butyl ester

To an at −7° C. stirred solution of 5 g (11.73 mmol) 2-(3-bromo-phenyl)-2-tert-butoxycarbonylamino-3,3,3-trifluoro-propionic acid ethyl ester in 50 ml toluene were added 58.7 ml of a 1.7M solution of DibalH in toluene. The mixture was stirred overnight at rt and quenched with an aqueous tartaric acid solution. The mixture was extracted with EtOAc and the organic phase was washed with brine, dried with MgSO4.H2O and evaporated. Purification via chromatography on silica gel (c-hexane/15-50% TBME) gave the desired product as a colorless resin.

TLC: Rf (Hexane/EtOAc 3:1)=0.35

HPLC: Rt_{H1}=3.155 min; ESIMS [M+Na]⁺=406/408(1Br);

¹H-NMR (CDCl3, 360 MHz): 7.54 (s, 1H), 7.45 (d, 1H), 7.35 (d, 1H), 7.23 (t, 1H), 5.38 (s, 1H), 4.28-4.12 (m, 3H), 1.38 (br s, 9H).

### c) [2-(3-Bromo-phenyl)-2-tert-butoxycarbonylamino-3,3,3-trifluoro-propoxy]-acetic acid ethyl ester

With the use of a syringe pump a solution of 1.87 ml (15.17 mmol) ethyl diazoacetate in 8 ml DCM were added to a stirred solution of 2.01 g (5.23 mmol) [1-(3-bromo-phenyl)-2,2,2-trifluoro-1-hydroxymethyl-ethyl]-carbamic acid tert-butyl ester and 46 mg (0.105 mmol) Rh₂ (OAc)₂ in 34 ml DCM over a period of 3.5 h. After 30 min the mixture was evaporated and chromatographed on silica gel (c-hexane/0-20% TBME) to give the desired product contaminated with a diazo ester oligomer.

HPLC: Rt_{H1}=3.752 min; ESIMS [M+Na]⁺=492/494(1Br);

¹H-NMR (CDCl3, 360 MHz): 7.67 (s, 1H), 7.51 (d, 1H), 7.46 (d, 1H), 7.28 (t, 1H), 6.28 (s, impurity), 6.05 (br s, 1H), 4.35-4.10 (m, 5H), 3.85 (br, 1H), 1.40 (br s, 9H), 1.35 (t, 3H).

### d) [2-Amino-2-(3-bromo-phenyl)-3,3,3-trifluoro-propoxy]-acetic acid ethyl ester

A solution of 1.4 g (2.47 mmol) [2-(3-bromo-phenyl)-2-tert-butoxycarbonylamino-3,3,3-trifluoro-propoxy]-acetic acid ethyl ester in 5 ml DCM was treated with 3.74 ml 4N HCl/dioxane. After standing overnight the mixture was evaporated, taken up in EtOAc and washed with 10% aqueous NaHCO3. The organic phase was washed with brine, dried with Na2SO4 and purified via chromatography on silica gel (c-hexane/10-15% EtOAc) to give the desired product as a colorless resin.

TLC: Rf (Hexane/EtOAc 3:1)=0.30.

HPLC: Rt_{H1}=2.316 min; ESIMS [M+H]⁺=370/372(1Br);

¹H-NMR (CDCl3, 360 MHz): 7.77 (s, 1H), 7.51 (d, 1H), 7.43 (d, 1H), 7.19 (t, 1H), 4.16 (q, 2H), 4.05 (s, 2H), 3.98 (d, 1H), 3.79 (d, 1H), 1.21 (t, 3H).

### e) 5-(3-Bromo-phenyl)-5-trifluoromethyl-morpholin-3-one

A solution of 598 mg (1.616 mmol) [2-Amino-2-(3-bromo-phenyl)-3,3,3-trifluoro-propoxy]-acetic acid ethyl ester in 5.4 ml toluene and 2.7 ml TFA was heated at reflux temperature for 5 h. The cooled down mixture was evaporated, taken up in EtOAc, washed with 5% aqueous NaHCO3, dried with Na2SO4 and evaporated to give the essentially pure title compound as a colorless solid.

TLC: Rf (Hexane/EtOAc 3:1)=0.13.

HPLC: Rt_{H1}=2.099 min; ESIMS [M+H]⁺=324/326(1Br);

¹H-NMR (CDCl3, 360 MHz): 7.68 (s, 1H), 7.63 (d, 1H), 7.48 (d, 1H), 7.39 (t, 1H), 6.70 (br s, 1H), 4.41 (d, 1H), 4.38 (d, 1H), 4.25 (d, 1H), 3.95 (d, 1H).

### f) [5-(3-Amino-phenyl)-5-trifluoromethyl-5,6-dihydro-2H-[1,4]oxazin-3-yl]-carbamic acid tert-butyl ester

The title compound was obtained by methods described for the conversions of 42e) to 42j).

TLC: Rf (Hexane/EtOAc 1:1)=0.16.

HPLC: Rt_{H4}=2.214 min; ESIMS [M+H]⁺=360;

¹H-NMR (CD3OD, 360 MHz): 7.00 (t, 1H), 6.86 (s, 1H), 6.78 (d, 1H), 6.60 (d, 1H), 4.52 (d, 1H), 4.40 (d, 1H), 4.03 (d, 1H), 3.88 (d, 1H).

### g) (5-{5-[(5-Chloro-pyridine-2-carbonyl)-amino]-2-fluoro-phenyl}-5-trifluoromethyl-5,6-dihydro-2H-[1,4]oxazin-3-yl)-carbamic acid tert-butyl ester

To a solution of 56 mg (0.156 mmol) [5-(3-amino-phenyl)-5-trifluoromethyl-5,6-dihydro-2H-[1,4]oxazin-3-yl]-carbamic acid tert-butyl ester, 27 mg (0.171 mmol) 5-chloro-pyridine-2-carboxylic acid, 27.6 mg (0.203 mmol) HOAt and 0.054 ml (0.39 mmol) Et3N in 1 ml DCM were added 33 mg (0.171 mmol) EDC.HCl. After 18 h the mixture was diluted with EtOAc, washed with water, 1N HCl, brine and 5% aqueous NaHCO3. The organic phase was dried with MgSO4.H2O, filtered and purified by chromatography on silica gel (hexane/14-18% EtOAc) to give the desired product as colorless foam.

TLC: Rf (Hexane/EtOAc 3:1)=0.34.

HPLC: Rt_{H4}=2.871 min; ESIMS [M+Na]⁺=521/523(1Cl);

¹H-NMR (CDCl3, 360 MHz): 8.51 (d, 1H), 8.19 (d, 1H), 7.85-7.80 (m, 3H), 7.36 (t, 1H), 7.25 (d, 1H), 4.68 (d, 1H), 4.57 (d, 1H), 4.15 (d, 1H), 4.03 (d, 1H), 1.50 (br s, 9H).

### h) 5-Chloro-pyridine-2-carboxylic acid [3-(5-amino-3-trifluoromethyl-3,6-dihydro-2H-[1,4]oxazin-3-yl)-4-fluoro-phenyl]-amide

A solution of 68 mg (0.136 mmol) (5-{5-[(5-chloro-pyridine-2-carbonyl)-amino]-2-fluoro-phenyl}-5-trifluoromethyl-5,6-dihydro-2H-[1,4]oxazin-3-yl)-carbamic acid tert-butyl ester in 2 ml 4N HCl in dioxane was stirred at 40° C. overnight. The mixture was concentrated and crystallized from EtOAc/hexane to yield the title compound as colorless crystals.

HPLC: Rt_{H3}=2.927 min; ESIMS [M+H]⁺=399/401(1Cl);

¹H-NMR (400 MHz, DMSO-d6): δ 10.60 (s, 1H), 8.19 (dd, 1H), 8.14 (d, 1H), 8.10 (s, 1H), 7.87 (d, 1H), 7.38 (d, 1H), 7.33 (d, 1H), 6.27 (br s, 1H), 4.12 (d, 1H), 4.04 (d, 1H), 3.94 (d, 1H), 3.93 (d, 1H).

### Example 51

### 5-Bromo-pyrimidine-2-carboxylic acid [3-((R)-5-amino-3-methyl-3,6-dihydro-2H-[1,4]oxazin-3-yl)-5-bromo-phenyl]-amide hydrochloride

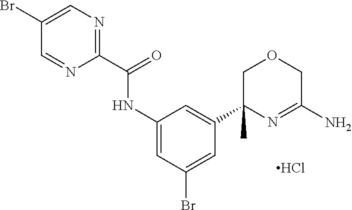 [SMILES: C[C@@]1(c2cc(Br)cc(NC(=O)c3ncc(Br)cn3)c2)COCC(N)=N1 ; Cl]

### a) 1-(3-Bromo-5-nitro-phenyl)-ethanol

A solution of TiCl₄ (9.48 g, 50 mmol) and methylmagnesiumbromide (20.80 ml, 52 mmol, 2.5 M solution in THF) in THF (400 ml) was stirred at −30° C. when 3-bromo-5-nitro-benzaldehyde (9.20 g, 40 mmol) was added as solid. The mixture was stirred for 1 h at −30° C. As the reaction was not complete, the reaction was cooled to −78° C. and 0.65 eq. methylmagnesium bromide and 0.625 eq. TiCl₄ were added and stirring was continued at −30° C. This procedure was repeated again to add 0.325 eq. methylmagnesium bromide and 0.313 eq. TiCl₄. After complete conversion the reaction was cooled to −78° C. and quenched by addition of 500 ml cold water. 500 ml dichloromethane were added and the reaction was allowed to warm to r.t. The phases were separated and the aqueous phase was extracted twice with dichloromethane. The organic phases were washed with water and brine, combined and dried over Na₂SO₄. Volatiles were removed under reduced pressure. The crude product was purified by automated column chromatography (cyclohexane/ethyl acetate) yielding the title compound as yellowish oil. ¹H-NMR (360 MHz, CDCl₃): 8.20 (s, 1H), 8.10 (s, 1H), 7.78 (s, 1H), 4.95 (q, 1H), 1.45 (d, 3H).

### b) 1-(3-Bromo-5-nitro-phenyl)-ethanone

1-(3-Bromo-5-nitro-phenyl)-ethanol (12.84 g, 52.2 mmol) was dissolved in dioxane (245 ml) and manganedioxide (31.8 g, 365 mmol) was added. The reaction was refluxed for 17 hrs. The reaction was filtered and solvent was removed under reduced pressure yielding the title compound as yellow solid. ¹H-NMR (500 MHz, DMSO-d₆): 8.64 (s, 1H), 8.58 (s, 1H), 8.53 (s, 1H), 2.70 (s, 3H); GC/MS: 243 [(M)⁺].

### c) 2-Methyl-propane-2-sulfinic acid [1-(3-bromo-5-nitro-phenyl)-eth-(E)-ylidene]-amide

1-(3-Bromo-5-nitro-phenyl)-ethanone (11.6 g, 47.5 mmol), (R)-(+)-tert-butanesulfinamide (6.34 g, 52.3 mmol) and Ti(OEt)₄ (24.64 ml, 119 mmol) were mixed in 62 ml THF and refluxed for 2.5 hrs. The reaction was cooled and carefully quenched by addition of ice and water. The white precipitate was filtered off and the aqueous mixture was extracted with ethyl acetate. The organic phases were washed with water and brine, combined and dried over Na₂SO₄. Volatiles were removed under reduced pressure. The crude product was purified by automated column chromatography (cyclohexane/ethyl acetate) yielding the title compound as yellow oil. ¹H-NMR (500 MHz, DMSO-d₆): 8.58 (s, 1H), 8.55 (s, 1H), 8.43 (s, 1H), 2.79 (s, 3H), 1.24 (s, 9H); MS: 347 [(M+H)⁺]; [α]^{D}=+54.5° (c=0.481% in chloroform).

### d) 2-Methyl-propane-2-sulfinic acid [(R)-(3-bromo-5-nitro-phenyl)-cyano-methyl-methyl]-amide

The sulfoxiimine from the previous step (12.48 g, 35.9 mmol) and CsF (6.01 g, 39.5 mmol) were dissolved in hexane (287 ml) and THF (72 ml) and cooled to −50° C. TMSCN (3.92 g, 39.5 mmol) were added dropwise and the reaction was stirred at 0° C. for 4 h. The reaction was cooled to −78° C. and quenched by addition of 720 ml saturated NH₄Cl solution. The product was extracted with ethyl acetate. The organic phases were washed with water and brine, combined and dried over Na₂SO₄. Volatiles were removed under reduced pressure. The crude product was purified by automated column chromatography (cyclohexane/ethyl acetate) yielding the title compound as tan solid. ¹H-NMR (500 MHz, CDCl₃): 8.43 (s, 2H), 8.13 (s, 1H), 4.19 (s, NH, 1H), 2.05 (s, 3H), 1.30 (s, 9H); MS: 374 [(M+H)⁺]; [α]^{D}=+3.2° (c=0.497% in chloroform)

### e) (R)-2-Amino-2-(3-bromo-5-nitro-phenyl)-propionic acid hydrochloride

2-Methyl-propane-2-sulfinic acid [(R)-(3-bromo-5-nitro-phenyl)-cyano-methyl-methyl]-amide (4.87 g, 13.0 mmol) was suspended in 215 ml conc. HCl (12.1 M) and refluxed for 4 hrs. Toluene was added twice and volatiles were removed under reduced pressure yielding a off-white solid. ¹H-NMR (360 MHz, MeOD): 8.60 (s, 1H), 8.44 (s, 1H), 8.18 (s, 1H), 2.05 (s, 3H); MS: 289 [(M+H)⁺].

### f) (R)-2-Amino-2-(3-bromo-5-nitro-phenyl)-propan-1-ol

(R)-2-Amino-2-(3-bromo-5-nitro-phenyl)-propionic acid (8.41 g, 25.8 mmol) was suspended in abs. THF (39 ml) and cooled to 0° C. BH₃ in THF (103 ml, 103 mmol, 1M in THF) was added and the reaction was stirred at r.t. for 1 hr. The reaction was poured onto NaHCO₃ (solid, 26 g, 12 eq.), 78 g ice and 155 ml ethyl acetate and stirred for 20 min. at r.t. Phases were separated. The organic phases were washed with water and brine, combined and dried over Na₂SO₄. Volatiles were removed under reduced pressure yielding the title compound as brown oil. ¹H-NMR (360 MHz, DMSO-d₆): 8.40 (s, 1H), 8.25 (s, 1H), 8.18 (s, 1H), 4.95 (t, 1H), 3.55 (m, 1H), 3.38 (m, 1H), 1.33 (s, 3H); MS: 275 [(M+H)⁺].

### g) N—[(R)-1-(3-Bromo-5-nitro-phenyl)-2-hydroxy-1-methyl-ethyl]-2-chloro-acetamide

(R)-2-Amino-2-(3-bromo-5-nitro-phenyl)-propan-1-ol (6.27 g, 22.79 mmol) was dissolved in DMF (230 ml) under N₂ and K₂CO₃ (7.87 g, 57 mmol) and DIPEA (3.98 ml, 22.79 mmol) were added. The mixture was cooled to 0° C. and chloro-acetylchloride (2.83 g, 25.07 mmol) was added dropwise. The reaction was stirred at 0° C. for 19 hrs. After completion, the reaction was put between water (2.3 liter) and toluene (2.3 l). The organic phases were washed with water and brine, combined and dried over Na₂SO₄. Volatiles were removed under reduced pressure. The crude product was purified by automated column chromatography (cyclohexane/ethyl acetate) yielding the title compound as colorless oil. ¹H-NMR (360 MHz, DMSO-d₆): 8.51 (1H, NH), 8.28 (s, 1H), 8.12 (s, 1H), 7.93 (s, 1H), 5.25 (t, 1H), 4.15 (d, 2H), 3.62 (m, 2H), 1.62 (s, 3H); MS: 351 [(M+H)⁺].

### h) (R)-5-(3-Bromo-5-nitro-phenyl)-5-methyl-morpholin-3-one

N—[(R)-1-(3-Bromo-5-nitro-phenyl)-2-hydroxy-1-methyl-ethyl]-2-chloro-acetamide (4.45 g, 10.76 mmol) and KOtBu (2.414 g, 21.52 g) were suspended in 55 ml tert-butanol under N₂ and heated 100° C. for 30 min. After completion water was added to the reaction and tert-butanol was removed under reduced pressure. The product was extracted with ethyl acetate from the remaining aqueous phase. The organic phases were washed with water and brine, combined and dried over Na₂SO₄. Volatiles were removed under reduced pressure. The crude product was purified by automated column chromatography (cyclohexane/ethyl acetate) yielding the title compound as tan solid. ¹H-NMR (360 MHz, DMSO-d₆): 8.88 (1H, NH), 8.35 (s, 1H), 8.28 (s, 1H), 8.14 (s, 1H), 4.16 (m, 1H), 4.06 (s, 2H), 3.74 (m, 1H), 1.50 (s, 3H); MS: 316 [(M+H)⁺].

### i) (R)-5-(3-Bromo-5-nitro-phenyl)-5-methyl-morpholine-3-thione

(R)-5-(3-Bromo-5-nitro-phenyl)-5-methyl-morpholin-3-one (2.60 g, 7.84 mmol) and Lawesson's reagent (2.54 g, 6.27 mmol) were stirred at 80° C. for 2 hrs. Volatiles were removed under reduced pressure and the crude product mixture was purified by automated column chromatography (cyclohexane/ethyl acetate) yielding the title compound as yellow foam. ¹H-NMR (360 MHz, DMSO-d₆): 11.28 (1H, NH), 8.38 (s, 1H), 8.22 (s, 1H), 8.17 (s, 1H), 4.44 (m, 1H), 4.22 (d, 1H), 3.81 (m, 1H), 1.60 (s, 3H); MS: 331 [(M+H)⁺].

### j) (R)-5-(3-Bromo-5-nitro-phenyl)-5-methyl-5,6-dihydro-2H-[1,4]oxazin-3-ylamine

(R)-5-(3-Bromo-5-nitro-phenyl)-5-methyl-morpholine-3-thione (2.86 g, 7.77 mmol) was dissolved in 7M NH3 in methanol (50 ml). Tert-Butylhydroperoxide (9.41 ml, 78 mmol) and ammonia hydroxide (25% sol., 21.2 ml, 136 mmol) were added and the reaction was stirred at r.t. for 2 hrs. Upon completion, 50 ml half-saturated Na₂S₂O₃ solution was added to the reaction and the product was extracted with ethyl acetate. The organic phases were washed with water and brine, combined and dried over Na₂SO₄. Volatiles were removed under reduced pressure, yielding the title compound as yellowish solid. ¹H-NMR (360 MHz, DMSO-d₆): 8.34 (s, 1H), 8.25 (s, 1H), 8.12 (s, 1H), 5.85 (br, 2H), 4.01 (m, 1H), 3.80 (m, 1H), 3.55 (m, 1H), 1.38 (s, 3H); MS: 314 [(M+H)⁺].

### k) [(R)-5-(3-Bromo-5-nitro-phenyl)-5-methyl-5,6-dihydro-2H-[1,4]oxazin-3-yl]-carbamic acid tert-butyl ester

(R)-5-(3-Bromo-5-nitro-phenyl)-5-methyl-5,6-dihydro-2H-[1,4]oxazin-3-ylamine (1.74 g, 5.10 mmol) was dissolved in 40 ml dichloromethane under N₂ and cooled to 0° C. Boc₂O (1.45 g, 6.63 mmol) and DIPEA (1.34 ml, 7.65 mmol) were added and the reaction was stirred at r.t for 18 hrs. 100 ml water was added to the reaction. The organic phases were washed with water and brine, combined and dried over Na₂SO₄. Volatiles were removed under reduced pressure. The crude product was purified by automated column chromatography (cyclohexane/ethyl acetate) yielding the title compound as white foam. ¹H-NMR (360 MHz, DMSO-d₆): 9.76 (s, 1H, NH), 8.32 (s, 1H), 8.27 (s, 1H), 8.17 (s, 1H), 4.60 (d, 1H), 4.38 (d, 1H), 3.94 (d, 1H), 3.48 (d, 1), 1.44 (s, 9H), 1.38 (s, 3H); MS: 414 [(M+H)⁺].

### l) [(R)-5-(3-Amino-5-bromo-phenyl)-5-methyl-5,6-dihydro-2H-[1,4]oxazin-3-yl]-carbamic acid tert-butyl ester

[(R)-5-(3-Bromo-5-nitro-phenyl)-5-methyl-5,6-dihydro-2H-[1,4]oxazin-3-yl]-carbamic acid tert-butyl ester (930 mg, 2.13 mmol) was dissolved in 5 ml methanol. Raney-Nickel was added and the reaction was hydrogenated for 1.5 hrs at r.t. The reaction was filtered over Celite, washed with dichloromethane/methanol (9/1). Volatiles were removed under reduced pressure yielding the title compound as white solid.

¹H-NMR (360 MHz, DMSO-d₆): 9.60 (br, 1H), 6.72 (s, 1H), 6.64 (s, 1H), 6.55 (s, 1H), 5.40 (br, 2H), 4.38 (m, 2H), 3.65 (m, 2H), 1.44 (s, 12H); MS: 384 [(M+H)⁺]; [α]^{D}=−172.9° (c=0.441% in methanol)

### m) ((R)-5-{3-Bromo-5-[(5-bromo-pyrimidine-2-carbonyl)-amino]-phenyl}-5-methyl-5,6-dihydro-2H-[1,4]oxazin-3-yl)-carbamic acid tert-butyl ester

[(R)-5-(3-Amino-5-bromo-phenyl)-5-methyl-5,6-dihydro-2H-[1,4]oxazin-3-yl]-carbamic acid tert-butyl ester (33 mg, 0.082 mmol), 5-bromo-pyrimidine-2-carboxylic acid (18 mg, 0.090 mmol) and HOBT (16 mg, 0.106 mmol) were dissolved in dichloromethane under N₂ at 0° C. DIPEA (10.54 mg, 0.082 mmol) and EDC (17 mg, 0.090 mmol) were added. The mixture was stirred at 0° C. for 10 min, then allowed to warm to room temperature, stirred for 17 h at room temperature, quenched with 1M aqueous KHCO₃ solution and extracted with dichloromethane. The organic phase was washed with water and brine, dried over Na₂SO₄ and concentrated under reduced pressure. The residue was purified by automated column chromatography (cyclohexane/ethyl acetate) to yield the title compound as a tan foam. MS: 569 [(M+H)⁺].

### n) 5-Bromo-pyrimidine-2-carboxylic acid [3-((R)-5-amino-3-methyl-3,6-dihydro-2H-[1,4]oxazin-3-yl)-5-bromo-phenyl]-amide hydrochloride

A solution of ((R)-5-{3-bromo-5-[(5-bromo-pyrimidine-2-carbonyl)-amino]-phenyl}-5-methyl-5,6-dihydro-2H-[1,4]oxazin-3-yl)-carbamic acid tert-butyl ester (22 mg, 0.039 mmol) in 4 M HCl in dioxane (0.8 ml, 80 eq.) was warmed to 40° C. for 24 hrs in a closed reaction vial. After completion volatiles were removed under reduced pressure to yield the title compound (hydrochloride salt) in the form of a colourless solid. ¹H-NMR (500 MHz, DMSO-d₆): 11.05 (s, 1H), 10.61 (s, 1H, NH⁺), 9.25 (s, 2H), 9.19 (s, 1H), 8.58 (s, 1H), 8.26 (s, 1H), 7.89 (s, 1H), 7.44 (s, 1H), 4.59 (m, 2H), 3.91 (m, 2H), 1.63 (s, 3H); MS: 470 [(M+H)⁺].

### Examples 52 to 59

The compounds listed in Table 5 were prepared by a procedure analogous to that used in example 51.

TABLE 5

| | | | MS |
|---|---|---|---|
| | | ¹H-NMR | [m/z; |
| Example | Compound | (δ; DMSO-d₆) | (M + 1)⁺] |
| 52 | 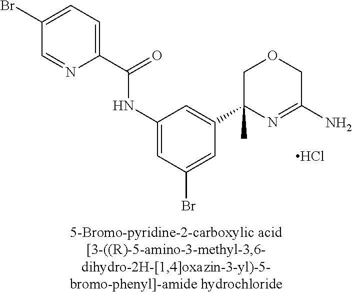 [SMILES: C[C@@]1(c2cc(Br)cc(NC(=O)c3ccc(Br)cn3)c2)COCC(N)=N1 ; Cl] | 10.45 (s, 1H), 8.88 (s, 1H), 8.35 (m, 1H), 8.17 (d, 1H), 8.07 (m, 1H), 7.93 (s, 1H), 7.68 (m, 2H), 7.40 (s, 1H), 4.55 (m, 2H), 4.15 (m, 2H), 1.60 (s, 3H) | 467 |
| 53 | 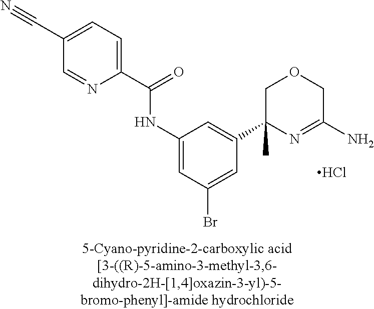 [SMILES: C[C@@]1(c2cc(Br)cc(NC(=O)c3ccc(C#N)cn3)c2)COCC(N)=N1 ; Cl] | 11.02 (s, 1H), 10.55 (s, 1H), 8.65 (s, 1H), 8.56 (m, 1H), 8.31 (s, 1H), 8.27 (m, 1H), 7.96 (s, 1H), 7.40 (s, 1H), 4.55 (m, 2H), 3.90 (m, 2H), 1.60 (s, 3H) | 414 |
| 54 | 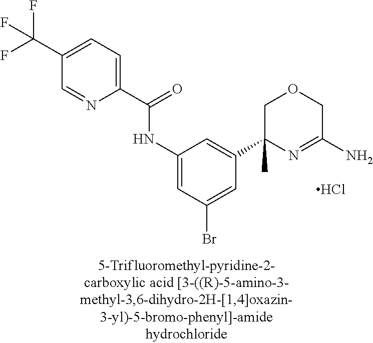 [SMILES: C[C@@]1(c2cc(Br)cc(NC(=O)c3ccc(C(F)(F)F)cn3)c2)COCC(N)=N1 ; Cl] | free base: 10.95 (s, 1H), 9.15 (s, 1H), 8.53 (m, 1H), 8.36 (m, 1H), 8.18 (s, 1H), 7.92 (s, 1H), 7.44 (s, 1H), 6.15 (br, 2H), 4.10 (m, 2H), 3.60 (m, 2H), 1.42 (s, 3H) | 457 |
| 55 | 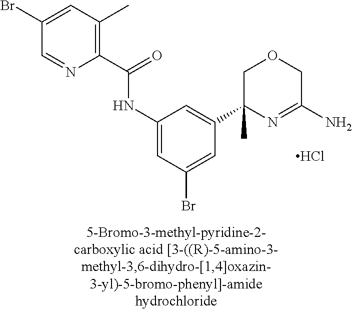 [SMILES: Cc1cc(Br)cnc1C(=O)Nc1cc(Br)cc([C@]2(C)COCC(N)=N2)c1 ; Cl] | 10.80 (s, 1H), 8.69 (s, 1H), 8.22 (m, 2H), 7.81 (s, 1H), 7.42 (s, 1H), 4.55 (br, 2H), 3.90 (m, 2H), 2.58 (s, 3H), 1.62 (s, 3H) | 481 |
| 56 | 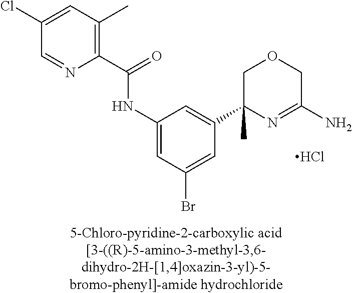 [SMILES: Cc1cc(Cl)cnc1C(=O)Nc1cc(Br)cc([C@]2(C)COCC(N)=N2)c1 ; Cl] | 10.90 (s, 1H), 8.82 (s, 1H), 8.30 (s, 1H), 8.25 (m, 1H), 8.18 (m, 1H), 7.96 (s, 1H), 7.42 (m, 2H), 4.55 (m, 2H), 3.90 (m, 2H), 1.63 (s, 3H) | 423 |
| 57 | 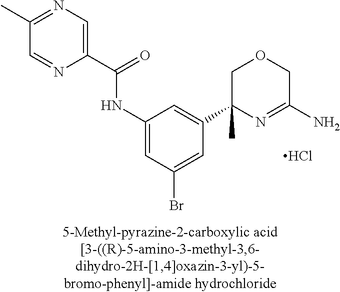 [SMILES: Cc1cnc(C(=O)Nc2cc(Br)cc([C@]3(C)COCC(N)=N3)c2)cn1 ; Cl] | 10.90 (s, 1H), 10.50 (br, 1H), 9.20 (s, 1H), 8.75 (s, 1H), 8.30 (s, 1H), 8.01 (s, 1H), 7.43 (s, 1H), 4.55 (m, 2H), 3.90 (m, 2H), 1.63 (s, 3H) | 405 |
| 58 | 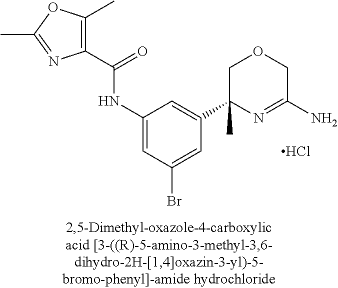 [SMILES: Cc1nc(C(=O)Nc2cc(Br)cc([C@]3(C)COCC(N)=N3)c2)c(C)o1 ; Cl] | 10.22 (s, 1H), 8.22 (s, 1H), 7.86 (s, 1H), 7.38 (s, 1H), 4.54 (m, 2H), 3.85 (m, 2H), 2.61 (s, 3H), 2.45 (s, 3H), 1.61 (s, 3H) | 406 |
| 59 | 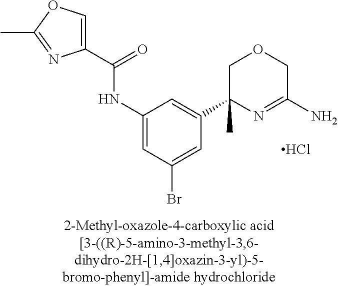 [SMILES: Cc1nc(C(=O)Nc2cc(Br)cc([C@]3(C)COCC(N)=N3)c2)co1 ; Cl] | 10.40 (s, 1H), 8.71 (s, 1H), 8.21 (s, 1H), 7.89 (s, 1H), 7.38 (s, 1H), 4.52 (m, 2H), 3.86 (m, 2H), 3.27 (s, 3H), 1.59 (s, 3H) | 392 |

### Examples 60 to 65

The compounds listed in Table 6 were prepared by procedures analogous to those used in examples 51 (steps a) and b)) and 1 (steps c) to l)). 5-Bromo-2-fluoro-benzaldehyde was used instead of 3-bromo-5-nitro-benzaldehyde.

TABLE 6

| | | | MS |
|---|---|---|---|
| | | ¹H-NMR | [m/z; |
| Example | Compound | (δ; DMSO-d₆) | (M + 1)⁺] |
| 60 | 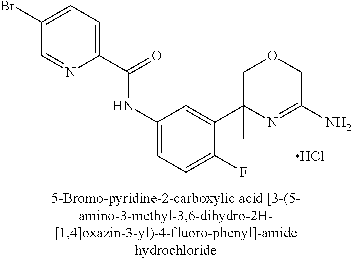 [SMILES: CC1(c2cc(NC(=O)c3ccc(Br)cn3)ccc2F)COCC(N)=N1 ; Cl] | 10.85 (s, 1H), 10.20 (br, 1H), 8.88 (s, 1H), 8.35 (m, 1H), 8.09 (d, 1H), 7.95 (m, 2H), 7.26 (t, 1H), 4.52 (m, 2H), 4.10 (m, 1H), 3.85 (m, 1H), 1.60 (s, 3H) | 407 |
| 61 | 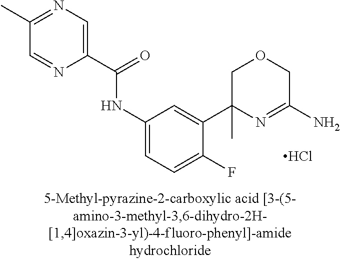 [SMILES: Cc1cnc(C(=O)Nc2ccc(F)c(C3(C)COCC(N)=N3)c2)cn1 ; Cl] | 10.85 (s, 1H), 10.25 (br, 1H), 9.30 (br, 1H), 9.15 (s, 1H), 8.72 (s, 1H), 8.58 (br, 1H), 8.00 (m, 2H), 7.28 (m, 1H), 4.55 (m, 2H), 4.12 (m, 1H), 3.88 (m, 1H), 1.62 (s, 3H) | 344 |
| 62 | 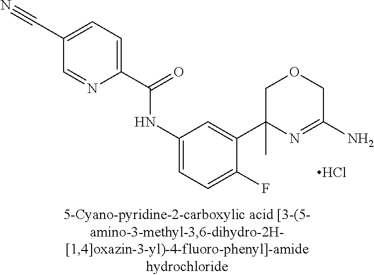 [SMILES: CC1(c2cc(NC(=O)c3ccc(C#N)cn3)ccc2F)COCC(N)=N1 ; Cl] | 11.02 (s, 1H), 10.70 (br, 1H), 9.20 (s, 1H), 8.59 (m, 1H), 8.31 (d, 1H), 7.98 (m, 2H), 7.27 (t, 1H), 4.52 (m, 2H), 4.06 (m, 1H), 3.87 (m, 1H), 1.64 (s, 3H) | 354 |
| 63 | 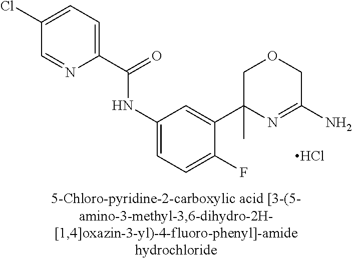 [SMILES: CC1(c2cc(NC(=O)c3ccc(Cl)cn3)ccc2F)COCC(N)=N1 ; Cl] | 10.95 (s, 1H), 10.80 (s, 1H), 9.32 (s, 1H), 8.80 (s, 1H), 8.62 (s, 1H), 8.20 (m, 1H), 8.14 (m, 1H), 8.03 (m, 1H), 7.95 (m, 1H), 7.26 (m, 1H), 4.58 (m, 2H), 4.12 (m, 1H), 3.88 (m, 1H), 1.67 (s, 3H) | 363 |
| 64 | 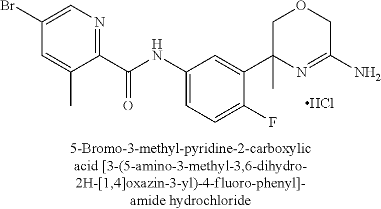 [SMILES: Cc1cc(Br)cnc1C(=O)Nc1ccc(F)c(C2(C)COCC(N)=N2)c1 ; Cl] | 10.75 (s, 1H), 10.72 (s, 1H), 9.30 (s, 1H), 8.66 (s, 1H), 8.63 (s, 1H), 8.18 (s, 1H), 7.94 (m, 1H), 7.81 (m, 1H), 7.28 (dd, 1H), 4.55 (m, 2H(AB- system)), 4.0 (m, 2H(AB-system)), 2.55 (s, 3H), 1.65 (s, 3H) | 421 |
| 65 | 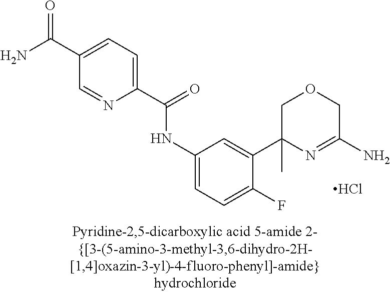 [SMILES: CC1(c2cc(NC(=O)c3ccc(C(N)=O)cn3)ccc2F)COCC(N)=N1 ; Cl] | 10.95 (s, 1H), 10.75 (s, 1H), 9.27 (s, 1H), 9.15 (s, 1H), 8.64 (s, 1H), 8.46 (m, 1H), 8.37 (m, 1H), 8.22 (m, 1H), 8.05 (m, 1H), 7.97 (m, 1H), 7.80 (s, 1H), 7.26 (m, 1H), 4.58 (m, 2H), 4.12 (m, 1H), 3.89 (m, 1H), 1.67 (s, 3H) | 372 |

### Example 66

### 5-Bromo-pyridine-2-carboxylic acid [3-((3R*,6R*)-5-amino-3,6-dimethyl-6-trifluoromethyl-3,6-dihydro-2H-[1,4]oxazin-3-yl)-phenyl]-amide hydrochloride

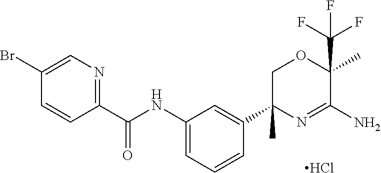 [SMILES: C[C@@]1(c2cccc(NC(=O)c3ccc(Br)cn3)c2)CO[C@@](C)(C(F)(F)F)C(N)=N1 ; Cl]

### a) 2-[2-(3-Bromo-phenyl)-2-oxo-ethoxy]-3,3,3-trifluoro-2-methyl-propionic acid methyl ester

To a solution of 3,3,3-trifluoro-2-hydroxy-2-methyl-propionic acid methyl ester (22.94 g, 133 mmol) in CH₂Cl₂ (400 ml) was added rhodium(II) trifluoroacetate dimer (0.439 g, 0.667 mmol). After cooling to 0° C. a solution of 1-(3-bromo-phenyl)-2-diazo-ethanone (15.0 g, 66.7 mmol) dissolved in CH₂Cl₂ (100 ml) was added over a period of 2 h. The reaction mixture was concentrated and the title compound was obtained after flash-chromatography on silica gel (toluene) as a yellow oil: TLC (toluene/EtOAc 10:1): Rf=0.45; HPLC Rt_{H5}=1.352 min; ¹H NMR (360 MHz, CDCl₃): δ 8.15 (s, 1H), 7.94 (d, 1H), 7.76 (d, 1H), 7.40 (t, 1H), 4.87 (s, 2H), 3.89 (s, 3H), 1.72 (s, 3H); ESIMS: 386, 388 [(M+NH₄)⁺].

### b) 2-[2-(3-Bromo-phenyl)-2-hydroxy-propoxy]-3,3,3-trifluoro-2-methyl-propionic acid methyl ester

To a solution of 2-[2-(3-bromo-phenyl)-2-oxo-ethoxy]-3,3,3-trifluoro-2-methyl-propionic acid methyl ester (6.6 g, 17 mmol) in toluene (120 ml) was added under argon at −78° C. a 2M solution of AlMe₃ in heptane (17 ml, 34 mmol) and after stirring for 0.5 h at −78° C. a 1.6 M solution of MeLi in Et₂O (21.3 ml, 34 mmol) over a period of 40 min. After stirring for 0.5 h at −78° C. the reaction mixture was added to a cold aqueous NaH₂PO₄ solution and was extracted with EtOAc. Combined organic layers were washed with brine, dried over MgSO₄, filtered and evaporated. The crude product was purified by flash-chromatography on silica gel (toluene to toluene/EtOAc 10:1) to provide a diastereomeric mixture of the title compound as a yellow oil: TLC (toluene/EtOAc 10:1): Rf=0.34 and 0.37; HPLC Rt_{H5}=1.359 min; ¹H NMR (360 MHz, CDCl₃): δ 7.69 (m, 1H), 7.43 (m, 2H), 7.25 (t, 1H), 3.86 (s, 3H), 3.68 (m, 2H), 3.43 and 3.33 (s, 1H), 1.63 and 1.61 (s, 3H), 1.58 (s, 3H); ESIMS: 402, 404 [(M+NH₄)⁺].

### c) 2-[2-Azido-2-(3-bromo-phenyl)-propoxy]-3,3,3-trifluoro-2-methyl-propionic acid methyl ester

To a solution of 2-[2-(3-bromo-phenyl)-2-hydroxy-propoxy]-3,3,3-trifluoro-2-methyl-propionic acid methyl ester (5.1 g, 11.92 mmol) in toluene (50 ml) was added trimethylsilyl azide (3.95 ml, 29.8 mmol) and at 0° C. BF₃-Et₂O (4.53 ml, 35.8 mmol). The reaction mixture was stirred for 2 days at 25° C. and for another day at 40° C. The reaction was carefully quenched by slow addition of the reaction mixture to a cold aqueous NH₄OH solution. The product was extracted with EtOAc and the combined organic layers were washed with brine, dried over MgSO₄, filtered and evaporated. The crude product was purified by flash-chromatography on silica gel (toluene to toluene/EtOAc 10:1) to provide a diastereomeric mixture of the title compound as a light yellow oil: TLC (toluene/EtOAc 10:1): Rf=0.69; HPLC Rt_{H5}=1.560 min; ¹H NMR (360 MHz, CDCl₃): δ 7.65 (s, 1H), 7.48 (d, 1H), 7.41 (d, 1H), 7.27 (t, 1H), 3.87 and 3.85 (s, 3H), 3.76 (m, 2H), 1.78 and 1.75 (s, 3H), 1.65 and 1.61 (s, 3H); ESIMS: 427, 429 [(M+NH₄)⁺].

### d) 2-[2-amino-2-(3-bromo-phenyl)-propoxy]-3,3,3-trifluoro-2-methyl-propionic acid methyl ester

To a solution of the 2-[2-azido-2-(3-bromo-phenyl)-propoxy]-3,3,3-trifluoro-2-methyl-propionic acid methyl ester (4.2 g, 9.22 mmol) in THF—H₂O 3:1 (48 ml) was added indium (2.116 g, 18.43 mmol) followed by 4N aqueous HCl over a period of 20 min and stirring for 1 h at 25° C. The reaction mixture was added to a 10% aqueous K₂CO₃ solution and the product was extracted with EtOAc. The combined organic layers were washed with brine, dried over MgSO₄, filtered and evaporated. The crude product was purified by flash-chromatography on NEt₃ deactivated silica gel (hexane/EtOAc 2:1 to EtOAc) to provide a diastereomeric mixture of the title compound as a yellow oil: TLC (EtOAc): Rf=0.46; HPLC Rt_{H5}=0.999 min; ¹H NMR (360 MHz, CDCl₃): δ 7.73 (s, 1H), 7.47 (d, 1H), 7.41 (d, 1H), 7.24 (t, 1H), 3.84 and 3.83 (s, 3H), 3.59 (s, 2H), 1.61 and 1.59 (s, 3H), 1.52 and 1.51 (s, 3H); ESIMS: 384, 386 [(M+H)⁺].

### e) (2R*,5R*)-5-(3-Bromo-phenyl)-2,5-dimethyl-2-trifluoromethyl-morpholin-3-one and (2S*,5R*)-5-(3-Bromo-phenyl)-2,5-dimethyl-2-trifluoromethyl-morpholin-3-one

To a solution of 2-[2-amino-2-(3-bromo-phenyl)-propoxy]-3,3,3-trifluoro-2-methyl-propionic acid methyl ester (2.7 g, 6.89 mmol) in CH₂Cl₂ (40 ml) was added under argon at 0-5° C. a 2M solution of AlMe₃ in heptane (10.33 ml, 20.66 mmol). After stirring for 1 h at 25° C. the reaction mixture was cannulated into a cold 1N aqueous HCl. The product was extracted with CH₂Cl₂ and the combined organic layers were washed with 5% aqueous NaHCO₃ solution, dried over MgSO₄, filtered and evaporated. The crude product was purified by flash-chromatography on silica gel (hexane/EtOAc 4:1 to 1:1) to provide the (2R*,5R*)-diastereomer as white crystals: TLC (hexane/EtOAc 3:1): Rf=0.34; HPLC Rt_{H5}=1.262 min; ¹H NMR (360 MHz, CDCl₃): δ 7.55 (s, 1H), 7.52 (m, 1H), 7.32 (m, 2H), 6.61 (s, 1H), 4.04 (s, 2H), 1.67 (s, 3H), 1.58 (s, 3H); ESIMS: 352, 354 [(M+H)⁺] and the (2S*,5R*)-diastereomer as white crystals: TLC (hexane/EtOAc 3:1): Rf=0.16; HPLC Rt_{H5}=1.230 min; ¹H NMR (360 MHz, CDCl₃): δ 7.57 (s, 1H), 7.52 (d, 1H), 7.37 (d, 1H), 7.32 (t, 1H), 6.45 (s, 1H), 4.11 (dd, 1H), 3.83 (dd, 1H), 1.7 (s, 3H), 1.72 (s, 3H); ESIMS: 352, 354 [(M+H)⁺].

### f) (2S*,5R*)-5-(3-Bromo-phenyl)-2,5-dimethyl-2-trifluoromethyl-morpholine-3-thione

To a solution of (2R*,5R*)-5-(3-bromo-phenyl)-2,5-dimethyl-2-trifluoromethyl-morpholin-3-one (2.48 g, 7.0 mmol) in toluene (25 ml) was added hexamethyldisiloxane (2.7 ml, 12.7 mmol) and phosphorouspentasulfide (1.878 g, 4.23 mmol) and the reaction mixture was heated at reflux for 16 h. To the cold reaction mixture was added acetone (10 ml) and 20% aqueous K₂CO₃ solution and the mixture was stirred for 1 h at 25° C. The product was extracted with EtOAc and the combined organic layers were washed with brine, dried over MgSO₄, filtered and concentrated. The crude product was crystallized from diisopropylether to provide the purified title compound as white crystals: TLC (hexane/EtOAc 3:1): Rf=0.55; HPLC Rt_{H5}=1.408 min; ¹H NMR (360 MHz, CDCl₃): δ 8.51 (s, 1H), 7.53 (d, 1H), 7.48 (s, 1H), 7.34 (t, 1H), 7.28 (d, 1H), 4.07 (m, 2H), 1.79 (s, 3H), 1.71 (s, 3H); ESIMS: 368, 370 [(M+H)⁺].

### g) (2R*,5R*)-5-(3-Bromo-phenyl)-2,5-dimethyl-2-trifluoromethyl-5,6-dihydro-2H-[1,4]oxazin-3-ylamine

To a solution of (2S*,5R*)-5-(3-bromo-phenyl)-2,5-dimethyl-2-trifluoromethyl-morpholine-3-thione (2.5 g, 6.79 mmol) in THF (25 ml) was added concentrated aqueous NH₄OH (10.7 ml, 170 mmol) and 80% tert-butylhydroperoxide in H₂O (4.25 ml, 33.9 mmol) and the reaction mixture was stirred for 3 h at 25° C. After addition of another 4.25 ml of 80% tert-butylhydroperoxide in H₂O the reaction mixture was stirred overnight at 25° C. The reaction mixture was slowly added to concentrated sodium metabisulfite solution at 0-10° C., and after addition of 20% aqueous K₂CO₃ solution the product was extracted with EtOAc. Combined organic layers were washed with brine, dried over MgSO₄, filtered and concentrated. The crude title product was used as such for the next transformation. A small amount was purified by flash-chromatography and transferred into the hydrochloride salt with 1N HCl in Et₂O to provide the title compound as a white solid: TLC (EtOAc/MeOH 9:1): Rf=0.60; HPLC Rt_{H5}=1.024 min; ¹H NMR (600 MHz, DMSO-d₆): δ 11.55 (s, 1H), 9.54 (d, 2H), 7.74 (s, 1H), 7.60 (d, 1H), 7.51 (d, 1H), 7.42 (t, 1H), 4.15 (d, 1H), 4.06 (d, 1H), 1.75 (s, 3H), 1.66 (s, 3H); ESIMS: 351, 353 [(M+H)⁺].

### h) [(2R*,5R*)-5-(3-Bromo-phenyl)-2,5-dimethyl-2-trifluoromethyl-5,6-dihydro-2H-[1,4]oxazin-3-yl]-carbamic acid tert-butyl ester

To a solution of (2R*,5R*)-5-(3-bromo-phenyl)-2,5-dimethyl-2-trifluoromethyl-5,6-dihydro-2H-[1,4]oxazin-3-ylamine (1.5 g, 4.27 mmol) in acetonitrile (30 ml) was added Boc₂O (1.86 g, 8.54 mmol) and NEt₃ (1.79 ml, 12.8 mmol) and the reaction mixture was stirred for 4 h at 25° C. The reaction mixture was added to aqueous NaH₃PO₄ solution and extracted with EtOAc. Combined organic layers were washed with brine, dried over MgSO₄, filtered and concentrated. The crude product was purified by flash-chromatography on silica gel (hexane to hexane/EtOAc 1:1) to provide the title compound as a light yellow oil: TLC (hexane/EtOAc 3:1): Rf=0.57; HPLC Rt_{H5}=1.549 min; ¹H NMR (360 MHz, CDCl₃): δ 7.50 (m, 2H), 7.30 (m, 2H), 4.04 (s, 2H), 1.69 (s, 3H), 1.64 (s, 3H), 1.57 (s, 9H); ESIMS: 451, 453 [(M+H)⁺].

### i) [(2R*,5R*)-5-(3-Azido-phenyl)-2,5-dimethyl-2-trifluoromethyl-5,6-dihydro-2H-[1,4]oxazin-3-yl]-carbamic acid tert-butyl ester

To a solution of [(2R*,5R*)-5-(3-bromo-phenyl)-2,5-dimethyl-2-trifluoromethyl-5,6-dihydro-2H-[1,4]oxazin-3-yl]-carbamic acid tert-butyl ester (1.1 g, 2.39 mmol) in EtOH—H₂O 2:1 (15 ml) was added under argon NaN₃ (0.62 g, 9.5 mmol), trans-N,N-dimethylcyclohexan-1,2-diamine (0.075 ml, 0.48 mmol), sodium-ascorbate (0.084 g, 0.48 mmol) and CuI (0.091 g, 0.48 mmol) and the resulting reaction mixture was heated for 45 min at 70° C. The reaction mixture was added to saturated aqueous NH₄Cl solution and extracted with EtOAc. Combined organic layers were washed with 5% aqueous NaHCO₃ solution and brine, dried over MgSO₄, filtered and concentrated. The crude title product was obtained as a yellow oil and used as such in the next transformation: TLC (toluene/EtOAc 10:1): Rf=0.53; HPLC Rt_{H5}=1.532 min; ¹H NMR (360 MHz, CDCl₃): δ 7.44 (t, 1H), 7.15 (d, 1H), 7.06 (d, 1H), 6.98 (s, 1H), 4.04 (m, 2H), 1.69 (s, 3H), 1.63 (s, 3H), 1.57 (s, 9H); ESIMS: 412, 414 [(M+H)⁺].

### j) [(2R*,5R*)-5-(3-Amino-phenyl)-2,5-dimethyl-2-trifluoromethyl-5,6-dihydro-2H-[1,4]oxazin-3-yl]-carbamic acid tert-butyl ester

A solution of [(2R*,5R*)-5-(3-azido-phenyl)-2,5-dimethyl-2-trifluoromethyl-5,6-dihydro-2H-[1,4]oxazin-3-yl]-carbamic acid tert-butyl ester (0.82 g, 1.92 mmol) in EtOH (10 ml) was stirred in the presence of 10% Pd—C (0.1 g) under an atmosphere of hydrogen at 25° C. for 1.5 h. The catalyst was filtered off over Celite, evaporated and the residual oil was purified by flash-chromatography on silica gel (hexane/EtOAc 10:1 to 1:2) to provide the title compound as a colorless foam: TLC (hexane/EtOAc 1:1): Rf=0.43; HPLC Rt_{H5}=1.082 min; ¹H NMR (360 MHz, CDCl₃): δ 10.94 (s, 1H), 7.21 (t, 1H), 6.74 (d, 1H), 6.67 (d, 1H), 6.66 (s, 1H), 4.01 (s, 2H), 3.78 (broad s, 2H), 1.68 (s, 3H), 1.65 (s, 3H), 1.57 (s, 9H); ESIMS: 386, 388 [(M+H)⁺].

### k) ((2R*,5R*)-5-{3-[(5-bromo-pyridine-2-carbonyl)-amino]-phenyl}-2,5-dimethyl-2-tri-fluoromethyl-5,6-dihydro-2H-[1,4]oxazin-3-yl)-carbamic acid tert-butyl ester

To a solution of [(2R*,5R*)-5-(3-amino-phenyl)-2,5-dimethyl-2-trifluoromethyl-5,6-dihydro-2H-[1,4]oxazin-3-yl]-carbamic acid tert-butyl ester (0.1 g, 0.253 mmol) in DMF (2.5 ml) was added 5-bromo-pyridine-2-carboxylic acid (78 mg, 0.379 mmol), EDC (0.074 g, 0.379 mmol), HOAt (0.053 g, 0.379 mmol) and DIPEA (0.083 g, 0.632 mmol) and the reaction mixture was stirred for 2 h at 25° C. After evaporation of the DMF the residue was taken up in NaH₂PO₄ solution and extracted with EtOAc. Combined organic layers were washed with 5% NaHCO₃ solution and brine, dried over MgSO₄, filtered and concentrated. The crude product was crystallized from diisopropylether to provide the title compound as a white crystalline solid: TLC (hexane/EtOAc 1:1): Rf=0.61; HPLC Rt_{H5}=1.565 min; ¹H NMR (360 MHz, CDCl₃): δ 11.01 (s, 1H), 9.92 (s, 1H), 8.71 (dd, 1H), 8.21 (d, 1H), 8.10 (dd, 1H), 7.87 (s, 1H), 7.73 (d, 1H), 7.46 (t, 1H), 7.18 (d, 1H), 4.10 (m, 2H), 1.75 (s, 3H), 1.67 (s, 3H), 1.58 (s, 9H); ESIMS: 571, 573 [(M+H)⁺].

### l) 5-Bromo-pyridine-2-carboxylic acid [3-((3R*,6R*)-5-amino-3,6-dimethyl-6-tri-fluoromethyl-3,6-dihydro-2H-[1,4]oxazin-3-yl)-phenyl]-amide hydrochloride

((2R*,5R*)-5-{3-[(5-bromo-pyridine-2-carbonyl)-amino]-phenyl}-2,5-dimethyl-2-trifluoro-methyl-5,6-dihydro-2H-[1,4]oxazin-3-yl)-carbamic acid tert-butyl ester (0.113 g, 0.194 mmol) was dissolved in THF (1 ml) and 4N HCl (5 ml) and the reaction mixture was stirred for 2 h at 25° C. and 3 h at 40° C. The solvents were removed under reduced pressure and the resulting residue was titurated with Et₂O to provide the title compound as a white amorphous solid:

TLC (EtOAc/MeOH 9:1): Rf=0.56; HPLC Rt_{H5}=1.147 min; ¹H NMR (600 MHz, DMSO-d₆): δ 11.45 (s, 1H), 10.81 (s, 1H), 9.74 (d, 2H), 8.88 (dd, 1H), 8.34 (dd, 1H), 8.10 (d, 1H), 8.01 (d, 1H), 7.95 (s, 1H), 7.46 (t, 1H), 7.27 (d, 1H), 4.13 (d, 1H), 4.05 (d, 1H), 1.81 (s, 3H), 1.69 (s, 3H); ESIMS: 471, 473 [(M+H)⁺].

### Example 67

The compound in Table 7 can be prepared by a procedure analogous to that used in example 66.

TABLE 7

| | | | MS |
|---|---|---|---|
| | | ¹H-NMR | [m/z; |
| Example | Compound | (δ; DMSO-d₆) | (M + 1)⁺] |
| 67 | 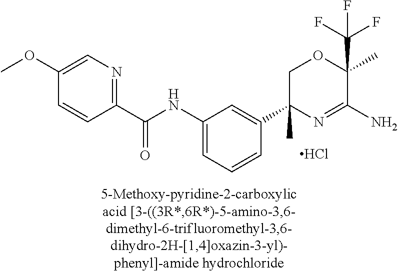 [SMILES: COc1ccc(C(=O)Nc2cccc([C@]3(C)CO[C@@](C)(C(F)(F)F)C(N)=N3)c2)nc1 ; Cl] | 11.65 (s, 1H), 10.65 (s, 1H), 9.55 (1, 1H), 9.51 (s, 1H), 8.40 (d, 1H), 8.14 (d, 1H), 8.01 (dd, 1H), 7.94 (s, 1H), 7.63 (dd, 1H), 7.44 (dd, 1H), 7.24 (dd, 1H), 4.18 (d, 1H), 4.07 (d, 1H), 3.90 (s, 3H), 1.78 (s, 3H), 1.58 (s, 3H) | 423 |

### Examples 68 to 70

The compounds listed in Table 8 can be prepared by a procedure analogous to that used in example 66, starting from 5-bromo-2-fluoro-benzoyl chloride.

TABLE 8

| | | | MS |
|---|---|---|---|
| | | ¹H-NMR | [m/z; |
| Example | Compound | (δ; DMSO-d₆) | (M + 1)⁺] |
| 68 | 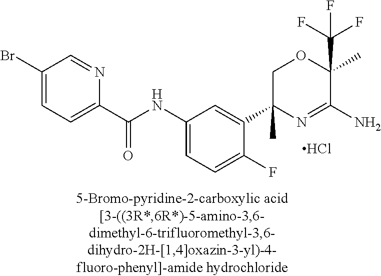 [SMILES: C[C@@]1(c2cc(NC(=O)c3ccc(Br)cn3)ccc2F)CO[C@@](C)(C(F)(F)F)C(N)=N1 ; Cl] | 11.65 (s, 1H), 11.00 (s, 1H), 9.60 (d, 2H), 8.84 (s, 1H), 8.34 (dd, 1H), 8.09 (d, 1H), 8.04 (m, 1H), 7.96 (dd, 1H), 7.33 (dd, 1H), 4.31 (d, 1H), 4.09 (d, 1H), 3.92 (s, 3H), 1.75 (s, 3H), 1.70 (s, 3H) | 489, 491 |
| 69 | 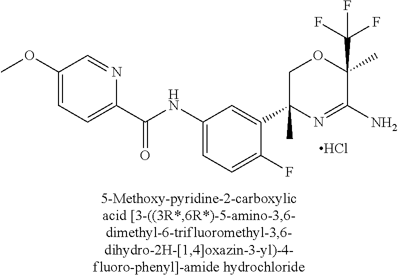 [SMILES: COc1ccc(C(=O)Nc2ccc(F)c([C@]3(C)CO[C@@](C)(C(F)(F)F)C(N)=N3)c2)nc1 ; Cl] | 11.68 (s, 1H), 10.81 (s, 1H), 9.59 (d, 2H), 8.40 (d, 1H), 8.12 (d, 1H), 8.02 (m, 1H), 7.97 (d, 1H), 7.62 (dd, 1H), 7.31 (dd, 1H), 4.29 (d, 1H), 4.09 (d, 1H), 3.92 (s, 3H), 1.77 (s, 3H), 1.70 (s, 3H) | 441 |
| 70 | 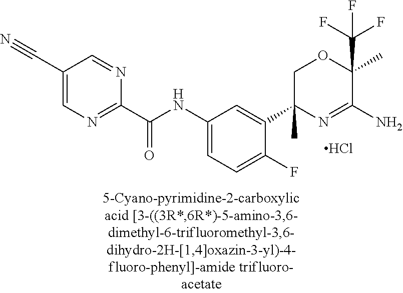 [SMILES: C[C@@]1(c2cc(NC(=O)c3ncc(C#N)cn3)ccc2F)CO[C@@](C)(C(F)(F)F)C(N)=N1 ; Cl] | 9.92 (br s, 1H), 8.0-9.1 (m, 5H), 7.89 (m, 1H), 7.51 (m, 1H), 7.09 (dd, 1H), 4.43 (d, 1H), 4.02 (d, 1H), 1.71 (s, 3H), 1.64 (s, 3H) | 437 |

### Example 71

### 5-Bromo-pyrimidine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide hydrochloride

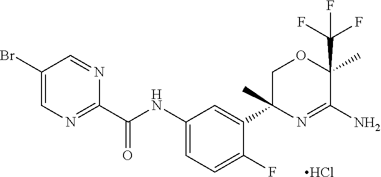 [SMILES: C[C@@]1(c2cc(NC(=O)c3ncc(Br)cn3)ccc2F)CO[C@@](C)(C(F)(F)F)C(N)=N1 ; Cl]

### a) 2-(5-Bromo-2-fluoro-phenyl)-propan-2-ol

To a solution of diisopropyl amine (57.3 ml, 402 mmol) in THF (500 ml) was added under argon a 1.6 M solution of nBuLi in hexane (260 ml, 416 mmol) below −50° C. After stirring for 30 min at −75° C., 4-bromo-1-fluoro benzene (31.1 ml, 277 mmol) was added while keeping the temperature below −70° C. After stirring for 2 h at −75° C., acetone (41.2 ml, 554 mmol) was added below −65° C. and the reaction mixture was stirred for 1 h at −75° C., warmed up to −50° C. and poured onto 10% aqueous NH₄Cl solution. The mixture was extracted with TBME, organic phases were washed with aqueous KHSO₄ solution, saturated NaHCO₃ solution and brine, dried over MgSO₄, filtered and concentrated. The crude product was crystallized from hexane to provide the title compound as white crystals: TLC (hexane-EtOAc 3:1): Rf=0.45; HPLC Rt_{H5}=1.045 min; ¹H NMR (360 MHz, CDCl₃): δ 7.74 (dd, 1H), 7.36 (m, 1H), 6.93 (dd, 1H), 2.04 (d, 1H), 1.63 (s, 6H).

### b) 4-Bromo-1-fluoro-2-isopropenyl-benzene

To a solution of 2-(5-bromo-2-fluoro-phenyl)-propan-2-ol (119.7 g, 498 mmol) in CH₂Cl₂ (50 ml) was added hydrochinone (2.74 g, 24.9 mmol) and 250 ml 85% H₃PO₄. The resulting reaction mixture was stirred for 3.5 h at 50° C. The mixture was poured onto ice-water and extracted with CH₂Cl₂. The organic phases were washed with 2N aqueous NaOH and water, dried over MgSO₄, filtered and concentrated. The crude product was dissolved in hexane and filtered through a plough of silica gel to obtain after concentration at 600 mbar the title compound as a colorless oil: TLC (hexane): Rf=0.52; HPLC Rt_{H5}=1.416 min; ¹H NMR (360 MHz, CDCl₃): δ 7.43 (dd, 1H), 7.37 (m, 1H), 6.94 (dd, 1H), 5.27 (d, 2H), 2.13 (s, 3H).

### c) (S)-2-(5-Bromo-2-fluoro-phenyl)-propane-1,2-diol

To a suspension of K₃Fe(CN)₆ (186 g, 561 mmol), K₂CO₃ (78 g, 561 mmol), (DHQ)₂-PHAL (1.311 g, 1.674 mmol) and K₂OsO₂(OH)₄ (0.378 g, 1 mmol) in t-BuOH—H₂O 1:1 (1600 ml) was added 4-bromo-1-fluoro-2-isopropenyl-benzene (36 g, 167 mmol) at 0° C. and the reaction mixture was stirred for 14 h at 0° C. After careful addition of Na₂S₂O₅ (100 g) at 0-5° C. the reaction mixture was stirred for 1 h before extraction with EtOAc. Combined extracts were washed with 5% NaS₃O₃ solution and brine, dried over MgSO4, filtered and concentrated to give the title compound as a white solid: TLC (hexane-EtOAc 1:1): Rf=0.46; HPLC Rt_{H5}=0.767 min; ESIMS: 266, 268 [(M+NH₄)⁺]; ¹H NMR (360 MHz, CDCl₃): δ 7.71 (dd, 1H), 7.27 (m, 1H), 6.83 (dd, 1H), 3.85 (d, 1H), 3.62 (d, 1H), 2.94 (s, 3H), 2.01 (s, 1H), 1.43 (s, 3H).

### d) (S)-2-(5-Bromo-2-fluoro-phenyl)-2-methyl-oxirane

To a solution of (S)-2-(5-bromo-2-fluoro-phenyl)-propane-1,2-diol (37.35 g, 150 mmol) in CH₂Cl₂ (400 ml) was added under argon NEt₃ (41.8 ml, 300 mmol) and dropwise mesyl chloride (12.8 ml, 165 mmol) at 0-5° C. After stirring for 0.5 h at 0-5° C. the reaction mixture was added to cold 1N HCl and extracted with CH₂Cl₂. Combined extracts were washed with 1N HCl, H₂O and saturated NaHCO₃ solution, dried over MgSO₄, filtered and concentrated. The crude mesylate was dissolved in TBME (500 ml) and 200 ml 2N aqueous NaOH and after stirring for 2 h at 25° C. the mixture was extracted with TBME.

Combined extracts were washed with NaH₂PO₄ solution and brine, dried over MgSO₄, filtered and concentrated to provide the (S)-enantiomer as a colorless oil: 78% ee (Chiralpak AS-H 1218, hexane-EtOH 97:3, 0.4 mL/min); TLC (hexane-EtOAc 3:1): Rf=0.69; HPLC Rt_{H5}=1.186 min; ¹H NMR (360 MHz, CDCl₃): δ 7.46 (dd, 1H), 7.30 (m, 1H), 6.83 (dd, 1H), 2.88 (d, 1H), 2.72 (d, 1H), 1.59 (s, 3H).

### e) (S)-1-Azido-2-(5-bromo-2-fluoro-phenyl)-propan-2-ol

To a solution of (S)-2-(5-bromo-2-fluoro-phenyl)-2-methyl-oxirane (51.85 g, 224 mmol) in EtOH (800 ml) was added NaN₃ (36.8 g, 531 mmol), NH₄Cl (60.6 g, 1122 mmol) and 18-crown-6 (59.8 g, 224 mmol) and the reaction mixture was heated at reflux for 6 h. The reaction mixture was filtered and concentrated to half of its volume. The residual oil was extracted with EtOAc. Combined extracts were washed with saturated NaHCO₃ solution and brine, dried over MgSO₄, filtered and concentrated to provide the title compound as a light yellow oil: TLC (hexane-EtOAc 1:1): Rf=0.70; HPLC Rt_{H3}=1.115 min; ¹H NMR (360 MHz, CDCl₃): δ 7.72 (dd, 1H), 7.32 (m, 1H), 6.85 (dd, 1H), 3.73 (d, 1H), 3.51 (d, 1H), 2.44 (s, 1H), 1.50 (s, 3H).

### f) (S)-1-Amino-2-(5-bromo-2-fluoro-phenyl)-propan-2-ol

To a suspension of LiAlH₄ (4.65 g, 122 mmol) in THF (250 ml) was added under argon at 0-5° C. a solution of (S)-1-azido-2-(5-bromo-2-fluoro-phenyl)-propan-2-ol (33.4 g, 122 mmol) dissolved in THF (150 ml) over a period of 30 min. After stirring for 1 h at 0-5° C., the reaction was quenched by careful addition of water (4.7 ml), 4 N NaOH (4.7 ml) and water (14.1 ml) and stirred again for 3 h at 25° C. The white suspension was dried with MgSO₄, filtered and concentrated. The solidified product was re-crystallized from TBME-hexane to provide the title compound as beige crystals: 98% ee (Chiralpak AD-H hexane-EtOH 75-25+0.05% NEt₃); TLC (CH₂Cl₂-MeOH 10:1) Rf=0.10; HPLC Rt_{H5}=0.558 min; ESIMS: 248, 250 [(M+H)⁺]; ¹H NMR (360 MHz, CDCl₃): δ 7.76 (dd, 1H), 7.25 (m, 1H), 6.82 (dd, 1H), 4.16 (br s, 1H), 3.19 (d, 1H), 2.72 (d, 1H), 1.44 (s, 3H), 0.95 (br s, 2H).

### g) N—[(S)-2-(5-Bromo-2-fluoro-phenyl)-2-hydroxy-propyl]-2-nitro-benzenesulfonamide

To a solution of (S)-1-amino-2-(5-bromo-2-fluoro-phenyl)-propan-2-ol (34.7 g, 140 mmol) in THF (400 ml) was added 2-nitro-benzenesulfonyl chloride (34.9 g, 154 mmol) at 0-5° C. and afterwards 1N aqueous NaOH over a period of 0.5 h. The reaction mixture was stirred for 2 h at 20° C. The reaction mixture was diluted with TBME and washed with water and NaH₂PO₄ solution and brine, dried over MgSO₄, filtered and concentrated to provide the title compound after crystallization from TBME-hexane as beige crystals: TLC (toluene-EtOAc 3:1): Rf=0.51; HPLC Rt_{H5}=1.118 min; ESIMS: 450, 452 [(M+NH₄)⁺]; ¹H NMR (360 MHz, CDCl₃): δ 7.98 (m, 1H), 7.81 (m, 1H), 7.65 (m, 2H), 7.59 (dd, 1H), 7.24 (m, 1H), 6.79 (dd, 1H), 5.60 (t, 1H), 4.16 (br s, 1H), 3.55 (dd, 1H), 3.44 (dd, 1H), 2.51 (s, 1H), 1.51 (s, 3H).

### h) (R)-2-(5-Bromo-2-fluoro-phenyl)-2-methyl-1-(2-nitro-benzenesulfonyl)-aziridine

To a solution of N—[(S)-2-(5-bromo-2-fluoro-phenyl)-2-hydroxy-propyl]-2-nitro-benzene-sulfonamide (20.8 g, 48 mmol) in CH₂Cl₂ (400 ml) was added PPh₃ (19.2 g, 72.4 mmol) at 0-5° C. and diethyl azodicarboxylate (11.6 ml, 72.4 mmol). The reaction mixture was stirred for 24 h at 25° C. and concentrated. The title compound was obtained after chromatographic purification over silica gel (hexane-EtOAc 20:1 to 2:1) as yellow crystals: TLC (toluene-EtOAc 3:1): Rf=0.69; HPLC Rt_{H5}=1.308 min; ¹H NMR (360 MHz, CDCl₃): δ 8.31 (m, 1H), 7.28 (m, 3H), 7.60 (dd, 1H), 7.42 (m, 1H), 6.91 (dd, 1H), 3.24 (s, 1H), 2.81 (s, 1H), 2.06 (s, 3H).

### i) (R)-2-[(R)-2-(5-Bromo-2-fluoro-phenyl)-2-(2-nitro-benzenesulfonylamino)-propoxy]-3,3,3-trifluoro-2-methyl-propionic acid ethyl ester

To a suspension of NaH (2.53 g 60% in mineral oil, 63 mmol) in DMF (160 ml) was added drop-wise under argon (R)-3,3,3-trifluoro-2-hydroxy-2-methyl-propionic acid ethyl ester (11.99 g, 63 mmol) and after stirring for 0.5 h at 20° C. (R)-2-(5-bromo-2-fluoro-phenyl)-2-methyl-1-(2-nitro-benzenesulfonyl)-aziridine (21.85 g, 52.6 mmol). The reaction was kept at 25° C. for 16 h. The mixture was added to cold aqueous 2N HCl and the product extracted with TBME. Combined organic layers were washed with saturated NaHCO₃ solution and brine, dried over MgSO₄, filtered and concentrated. The residual solid was re-crystallized from TBME-hexane to provide the title compound as yellow crystals: TLC (hexane-EtOAc 1:1): Rf=0.59; HPLC Rt_{H5}=1.444 min; ESIMS: 618, 620 [(M+NH₄)⁺]; ¹H NMR (360 MHz, CDCl₃): δ 7.83 (dd, 1H), 7.61 (m, 3H), 7.48 (dd, 1H), 7.27 (m, 1H), 6.73 (s, 1H), 6.60 (dd, 1H), 4.33 (m, 2H), 3.84 (s, 2H), 1.84 (s, 3H), 1.57 (s, 3H), 1.33 (t, 3H).

### j) (R)-2-[(R)-2-(5-Bromo-2-fluoro-pheyl)-2-(2-nitro-benzenesulfonylamino)-propoxy]-3,3,3-trifluoro-2-methyl-propionamide

A solution of (R)-2-[(R)-2-(5-bromo-2-fluoro-phenyl)-2-(2-nitro-benzenesulfonylamino)-propoxy]-3,3,3-trifluoro-2-methyl-propionic acid ethyl ester (26.6 g, 44.2 mmol) in 7N NH₃ in MeOH (75 ml) was stirred for 16 h at 50° C. The solvent was removed under reduced pressure and the residual solid re-crystallized from Et₂O to give the title compound as yellow crystals: TLC (hexane-EtOAc 1:1): Rf=0.35; HPLC Rt_{H5}=1.184 min; ESIMS: 589, 591 [(M+NH₄)⁺]; ¹H NMR (360 MHz, CDCl₃): δ 7.85 (d, 1H), 7.64 (m, 3H), 7.44 (d, 1H), 7.41 (dd, 1H), 7.26 (m, 1H), 6.68 (br s, 1H), 6.57 (dd, 1H), 6.19 (s, 1H), 5.54 (br s, 1H), 4.24 (d, 1H), 3.93 (d, 1H), 1.79 (s, 3H), 1.67 (s, 3H).

### k) N—[(R)-1-(5-Bromo-2-fluoro-phenyl)-2-((R)-1-cyano-2,2,2-trifluoro-1-methyl-ethoxy)-1-methyl-ethyl]-2-nitro-benzenesulfonamide

To a solution of (R)-2-[(R)-2-(5-bromo-2-fluoro-pheyl)-2-(2-nitro-benzenesulfonylamino)-propoxy]-3,3,3-trifluoro-2-methyl-propionamide (20.83 g, 35.6 mmol) in CH₂Cl₂ (300 ml) was added under argon NEt₃ (12.5 ml, 89 mmol) and at 0-5° C. trifluoroacetic anhydride (6.15 ml, 42.7 mmol). After stirring for 4 h at 25° C. the reaction mixture was added to a cold NaHCO₃ solution and the product was extracted with CH₂Cl₂. Combined extracts were washed with cold 0.1 N aqueous HCl, water and saturated NaHCO₃ solution, dried over MgSO₄, filtered and concentrated to provide the title compound as a yellow oil, which was used as such for the next step: TLC (hexane-EtOAc 1:1): Rf=0.73; HPLC Rt_{H5}=1.364 min; ESIMS: 571, 573 [(M+NH₄)⁺]; ¹H NMR (360 MHz, CDCl₃): δ 7.89 (d, 1H), 7.62 (ddd, 1H), 7.57 (ddd, 1H), 7.52 (m, 2H), 7.29 (m, 1H), 6.58 (dd, 1H), 6.19 (s, 1H), 4.17 (s, 2H), 1.81 (s, 3H), 1.72 (s, 3H).

### l) (2R,5R)-5-(5-Bromo-2-fluoro-phenyl)-2,5-dimethyl-2-trifluoromethyl-5,6-dihydro-2H-[1,4]oxazin-3-ylamine

To a solution of N—[(R)-1-(5-bromo-2-fluoro-phenyl)-2-((R)-1-cyano-2,2,2-trifluoro-1-methyl-ethoxy)-1-methyl-ethyl]-2-nitro-benzenesulfonamide (6.54 g, 11.8 mmol) and N-acetyl-cysteine (2.4 g, 26.0 mmol) in MeOH (80 ml) was added K₂CO₃ (3.62 g, 26.0 mmol) and the reaction mixture was heated at 80° C. for 16 h. After removal of the solvent the residue was dissolved in water and extracted with EtOAc. Combined extracts were washed with saturated NaHCO₃ solution and brine, dried over MgSO₄, filtered and concentrated to provide the title compound after chromatographic purification over silica gel (hexane-EtOAc 10:1 to 1:2 containing 0.03% NEt₃) as a yellow oil: TLC (hexane-EtOAc 1:1): Rf=0.58; HPLC Rt_{H5}=0.843 min; ESIMS: 369, 371 [(M+H)⁺]; ¹H NMR (360 MHz, CDCl₃): δ 7.66 (dd, 1H), 7.35 (m, 1H), 6.91 (dd, 1H), 3.97 (m, 2H), 1.53 (s, 3H), 1.49 (s, 3H).

### m) (2R,5R)-5-(2-Fluoro-phenyl)-2,5-dimethyl-2-trifluoromethyl-5,6-di hydro-2H-[1,4]oxazin-3-ylamine

A solution of (2R,5R)-5-(5-bromo-2-fluoro-phenyl)-2,5-dimethyl-2-trifluoromethyl-5,6-dihydro-2H-[1,4]oxazin-3-ylamine (1.66 g, 4.5 mmol) and sodium acetate (0.369 g, 4.5 mmol) in MeOH (50 ml) was hydrogenated over 10% Pd—C for 6 h at 50° C. The catalyst was filtered off over Celite and the filtrate was concentrated. The residue was dissolved in saturated NaHCO₃ solution and extracted with EtOAc. Combined extracts were washed with brine, dried over MgSO₄, filtered and concentrated to provide the title compound as a colorless oil: TLC (hexane-EtOAc 1:1): Rf=0.19; HPLC Rt_{H5}=0.777 min; ESIMS: 291 [(M+H)⁺]; ¹H NMR (360 MHz, CDCl₃): δ 7.41 (dt, 1H), 7.26 (m, 1H), 7.11 (t, 1H), 7.05 (dd, 1H), 4.11 (dd, 1H), 3.94 (dd, 1H), 1.54 (s, 3H), 1.49 (s, 3H).

### n) (2R,5R)-5-(2-Fluoro-5-nitro-phenyl)-2,5-dimethyl-2-trifluoromethyl-5,6-dihydro-2H-[1,4]oxazin-3-ylamine

To a solution of (2R,5R)-5-(2-fluoro-phenyl)-2,5-dimethyl-2-trifluoromethyl-5,6-dihydro-2H-[1,4]oxazin-3-ylamine (1.035 g, 3.57 mmol) in H₂SO₄ (6 ml) was added in portions KNO₃ (0.379 g, 3.74 mmol) under ice-water cooling. The reaction mixture was stirred for 2 h at 25° C., diluted with water and basified with K₂CO₃ under cooling. The product was extracted with EtOAc. Combined extracts were washed with saturated NaHCO₃ solution and brine, dried over MgSO₄, filtered and concentrated. Purification via chromatography on silica gel (hexane-EtOAc 4:1 to 1:1 containing 0.05% NEt₃) gave the title compound as a light yellow oil: TLC (hexane-EtOAc 1:1): Rf=0.50; HPLC Rt_{H5}=0.749 min; ESIMS: 336 [(M+H)⁺]; ¹H NMR (360 MHz, CDCl₃): δ 8.48 (dd, 1H), 8.14 (m, 1H), 7.15 (dd, 1H), 4.20 (br s, 2H), 4.04 (dd, 1H), 3.91 (dd, 1H), 1.54 (s, 3H), 1.49 (s, 3H).

### o) [(2R,5R)-5-(2-Fluoro-5-nitro-phenyl)-2,5-dimethyl-2-trifluoromethyl-5,6-dihydro-2H-[1,4]oxazin-3-yl]-carbamic acid tert-butyl ester

To a solution of (2R,5R)-5-(2-fluoro-5-nitro-phenyl)-2,5-dimethyl-2-trifluoromethyl-5,6-di-hydro-2H-[1,4]oxazin-3-ylamine (1.14 g, 3.4 mmol) in ACN (20 ml) was added Boc₂O (0.891 g, 4.08 mmol) and NEt₃ (0.72 ml, 5.1 mmol) and the mixture was stirred for 16 h at 25° C. The reaction mixture was evaporated and the residual oil purified by chromatography on silica gel (hexane-EtOAc 20:1 to 7:3) to give the title compound after crystallization from Et₂O-hexane as beige crystals: TLC (hexane-EtOAc 3:1): Rf=0.37; HPLC Rt_{H5}=1.355 min; ESIMS: 436 [(M+H)⁺]; ¹H NMR (360 MHz, CDCl₃): δ 11.04 (br s, 1H), 8.24 (m, 2H), 7.30 (dd, 1H), 4.41 (dd, 1H), 4.11 (dd, 1H), 1.68 (s, 3H), 1.51 (s, 9H), 1.49 (s, 3H).

### p) [(2R,5R)-5-(5-Amino-2-fluoro-phenyl)-2,5-di methyl-2-trifluoromethyl-5,6-di hydro-2H-[1,4]oxazin-3-yl]-carbamic acid tert-butyl ester

A solution of [(2R,5R)-5-(2-fluoro-5-nitro-phenyl)-2,5-dimethyl-2-trifluoromethyl-5,6-dihydro-2H-[1,4]oxazin-3-yl]-carbamic acid tert-butyl ester (0.98 g, 2.25 mmol) in isopropanol-THF 2:1 (24 ml) was hydrogenated over 5% Pd—C for 4 h at 50° C. The catalyst was filtered off over Celite and the filtrate was concentrated to provide the title compound after crystallization from TBME-hexane as beige crystals: TLC (hexane-EtOAc 1:1): Rf=0.42; HPLC Rt_{H5}=0.955 min; ESIMS: 406 [(M+H)⁺]; ¹H NMR (360 MHz, CDCl₃): δ 6.82 (dd, 1H), 6.52 (m, 2H), 4.30 (dd, 1H), 3.97 (dd, 1H), 3.06 (br s, 2H), 1.58 (s, 3H), 1.48 (s, 3H), 1.46 (s, 9H).

### q) ((2R,5R)-5-{5-[(5-Bromo-pyrimidine-2-carbonyl)-amino]-2-fluoro-phenyl}-2,5-di-methyl-2-trifluoromethyl-5,6-dihydro-2H-[1,4]oxazin-3-yl)-carbamic acid tert-butyl ester

To a solution of [(2R,5R)-5-(5-amino-2-fluoro-phenyl)-2,5-dimethyl-2-trifluoromethyl-5,6-dihydro-2H-[1,4]oxazin-3-yl]-carbamic acid tert-butyl ester (76 mg, 0.187 mmol) in DMF (2 ml) was added 5-bromopyridine-2-carboxylic acid (47 mg, 0.225 mmol), EDC.HCl (48 mg, 0.244 mmol), HOAt (29 mg, 0.206 mmol) and DIPEA (0.08 ml, 0.469 mmol) and the reaction mixture was kept at 25° C. for 16 h. The mixture was concentrated, the residue dissolved in EtOAc and washed with saturated NaHCO₃ solution and brine, dried over MgSO₄, filtered and purified by chromatography on silica gel (hexane-EtOAc 20:1 to 1:1) to provide the title compound as a light yellow foam: HPLC Rt_{H5}=1.297 min); ESIMS: 590, 592 [(M+H)⁺]; ¹H NMR (360 MHz, CDCl₃): δ 10.98 (br s, 1H), 9.71 (br s, 1H), 8.94 (s, 2H), 7.89 (m, 1H), 7.49 (dd, 1H), 7.12 (dd, 1H), 4.38 (d, 1H), 4.04 (d, 1H), 1.66 (s, 3H), 1.56 (s, 3H), 1.52 (s, 9H).

### r) 5-Bromo-pyrimidine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide hydrochloride

A solution of ((2R,5R)-5-{5-[(5-bromo-pyrimidine-2-carbonyl)-amino]-2-fluoro-phenyl}-2,5-dimethyl-2-trifluoromethyl-5,6-dihydro-2H-[1,4]oxazin-3-yl)-carbamic acid tert-butyl ester (90 mg, 0.153 mmol) in 4N HCl in dioxane (1 ml) was stirred at 40-45° C. for 6 h. The mixture was concentrated and the residue crystallized from Et₂O to yield the title compound as a beige solid: HPLC Rt_{H5}=0.837 min); ESIMS: 490,492 [(M+H)⁺]; ¹H NMR (600 MHz, DMSO-d₆): 11.61 (br s, 1H), 11.14 (br s, 1H), 9.61 (br s, 2H), 9.26 (s, 2H), 7.98 (d, 1H), 7.90 (d, 1H), 7.32 (dd, 1H), 4.31 (d, 1H), 4.10 (d, 1H), 1.72 (s, 3H), 1.62 (s, 3H).

### Examples 72 to 74

The compounds listed in Table 9 can be prepared by procedures analogous to those used in examples 71 and 72.

TABLE 9

| | | | MS |
|---|---|---|---|
| | | ¹H-NMR | [m/z; |
| Example | Compound | (δ; DMSO-d₆) | (M + 1)⁺] |
| 72 | 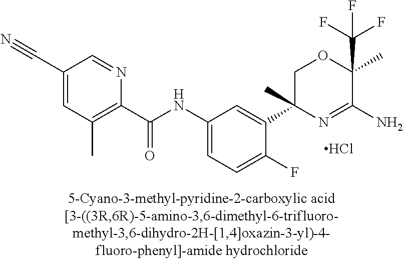 [SMILES: Cc1cc(C#N)cnc1C(=O)Nc1ccc(F)c([C@]2(C)CO[C@@](C)(C(F)(F)F)C(N)=N2)c1 ; Cl] | 11.0 (s, 1H), 9.60 (d, 2H), 9.04 (s, 1H), 8.41 (s, 1H), 7.96 (m, 1H), 7.83 (dd, 1H), 7.33 (dd, 1H), 4.37 (d, 1H), 4.11 (d, 1H), 2.56 (s, 3H), 1.73 (s, 3H), 1.70 (s, 3H) | 450 |
| 73 | 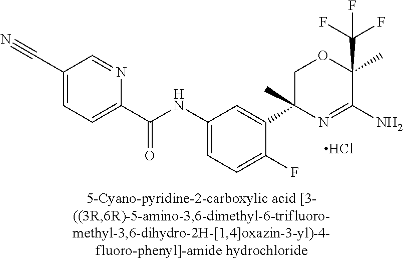 [SMILES: C[C@@]1(c2cc(NC(=O)c3ccc(C#N)cn3)ccc2F)CO[C@@](C)(C(F)(F)F)C(N)=N1 ; Cl] | 9.65 (d, 2H), 9.22 (s, 1H), 8.06 (d, 1H), 8.29 (d, 1H), 8.07 (m, 1H), 7.98 (m, 1H), 7.35 (dd, 1H), 4.34 (d, 1H), 4.10 (d, 1H), 1.75 (s, 3H), 1.71 (s, 3H) | 436 |
| 74 | 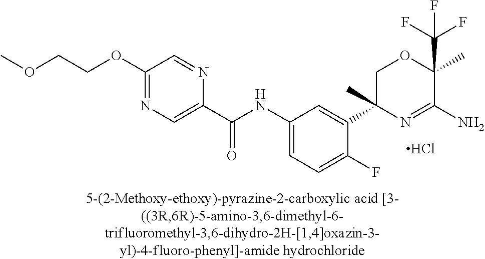 [SMILES: COCCOc1cnc(C(=O)Nc2ccc(F)c([C@]3(C)CO[C@@](C)(C(F)(F)F)C(N)=N3)c2)cn1 ; Cl] | 10.82 (br s, 1H), 9.58 (br s, 2H), 8.86 (s, 1H), 8.46 (s, 1H), 8.00 (m, 2H), 7.28 (m, 1H), 4.52 (br s, 2H), 4.31 (m, 1H), 4.08 (m, 1H), 3.72 (br s, 2H), 3.33 (s, 3H), 1.70 (s, 3H), 1.66 (s, 3H) | 486 |

### More detailed description of preparation of Example 72

### 5-Cyano-3-methyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide hydrochloride

### a) ((2R,5R)-5-{5-[(5-Cyano-3-methyl-pyridine-2-carbonyl)-amino]-2-fluoro-phenyl}-2,5-dimethyl-2-trifluoromethyl-5,6-di hydro-2H-[1,4]oxazin-3-yl)-carbamic acid tert-butyl ester

To a solution of [(2R,5R)-5-(5-amino-2-fluoro-phenyl)-2,5-dimethyl-2-trifluoromethyl-5,6-dihydro-2H-[1,4]oxazin-3-yl]-carbamic acid tert-butyl ester (82 mg, 0.20 mmol) in DMF (2 ml) was added 5-cyano-3-methyl-pyridine-2-carboxylic acid [Acid-3] (42 mg, 0.26 mmol), EDC.HCl (51 mg, 0.26 mmol), HOAt (31 mg, 0.22 mmol) and DIPEA (0.09 ml, 0.52 mmol) and the reaction mixture was kept at 25° C. for 16 h. The mixture was concentrated, the residue dissolved in EtOAc and washed with saturated NaHCO₃ solution and brine, dried over MgSO₄, filtered and purified by chromatography on silica gel (hexane-EtOAc 20:1 to 1:1) to provide the title compound as a light yellow foam: TLC (hexane-EtOAc 1:1): Rf=0.81; HPLC Rt_{H5}=1.437 min; ESIMS: 550 [(M+H)⁺]; ¹H NMR (360 MHz, CDCl₃): δ 10.96 (br s, 1H), 9.95 (br s, 1H), 8.63 (s, 2H), 7.88 (m, 1H), 7.71 (m, 1H), 7.54 (dd, 1H), 7.08 (dd, 1H), 4.34 (d, 1H), 4.02 (d, 1H), 2.77 (s, 3H), 1.63 (s, 3H), 1.47 (m, 12H).

### b) 5-Cyano-3-methyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide hydrochloride

To a solution of ((2R,5R)-5-{5-[(5-cyano-3-methyl-pyridine-2-carbonyl)-amino]-2-fluoro-phenyl}-2,5-dimethyl-2-trifluoromethyl-5,6-dihydro-2H-[1,4]oxazin-3-yl)-carbamic acid tert-butyl ester in CH₂Cl₂ (0.3 ml) was added TFA (0.6 ml) and the reaction mixture was kept at 25° C. for 2 h. The reaction was added to cold 10% aqueous K₂CO₃ solution and the product extracted with EtOAc. Combined organic layers were washed with brine, dried over MgSO₄, filtered and concentrated to provide 5-cyano-3-methyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide as a colorless foam. The title compound was converted into its hydrochloride salt by dissolving the free base in CH₂Cl₂, adding 1 eq of 2N HCl in Et₂O, evaporation to dryness, followed by crystallization from CH₂Cl₂-Et₂O to provide the title compound as a white solid: HPLC Rt_{H5}=0.957 min; ESIMS: 450 [(M+H)⁺]; ¹H NMR (600 MHz, DMSO-d₆): 11.0 (s, 1H), 9.60 (d, 2H), 9.04 (s, 1H), 8.41 (s, 1H), 7.96 (m, 1H), 7.83 (dd, 1H), 7.33 (dd, 1H), 4.37 (d, 1H), 4.11 (d, 1H), 2.56 (s, 3H), 1.73 (s, 3H), 1.70 (s, 3H).

### Example 75

### 5-Cyano-pyridine-2-carboxylic acid [3-((3R*,6R*)-5-amino-3-difluoromethyl-6-methyl-3,6-dihydro-2H-[1,4]oxazin-3-yl)-4-fluoro-phenyl]-amide hydrochloride

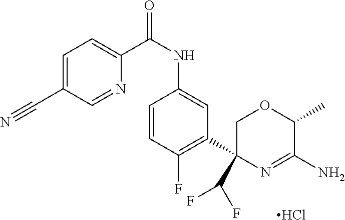 [SMILES: C[C@H]1OC[C@](c2cc(NC(=O)c3ccc(C#N)cn3)ccc2F)(C(F)F)N=C1N ; Cl]

### a) N-[1-(5-Bromo-2-fluoro-phenyl)-2,2-difluoro-1-hydroxymethyl-ethyl]-2-chloro-propionamide

[1-(5-Bromo-2-fluoro-phenyl)-2,2-difluoro-1-hydroxymethyl-ethyl]-carbamic acid tert-butyl ester [Example 42 step c)] (2.21 g, 5.75 mmol) was dissolved in 20 mL HCl solution 4 mol/L in dioxane and stirred at room temperature for 60 minutes. The reaction mixture was evaporated to give a white solid which was directly taken up in 15 mL dichloromethane. 20 mL aqueous Na₂CO₃ solution (10% w/w) was added and the emulsion was cooled to 0-5° C. Racemic 2-chloro-propionyl chloride (787 mg, 6.20 mmol) was added dropwise and the reaction mixture was slowly warmed to room temperature. After 30 minutes, the layers were separated and washed with dichloromethane. The organic layers were combined, dried over Na₂SO₄ and evaporated. The crude product was purified on a silica gel column by eluting with heptanes/EtOAc 3/1->2/1 to give 632 mg of the first eluting and 619 mg of the second eluting diastereomer.

Analytical data of first eluting diastereomer:

HPLC Rt_{H1}=2.403 min; ESIMS [M+H]⁺=374, 376 (1Br);

¹H-NMR (CDCl₃, 360 MHz): 7.56-7.46 (m, 2H), 7.39 (dd, 1H), 7.06 (dd, 1H), 6.35 (t, J=54 Hz, 1H), 4.64-4.56 (dd, 1H), 4.40-4.29 (m, 1H), 4.21-4.14 (m, 1H), 4.07-4.00 (dd, 1H), 1.86 (d, 3H).

Analytical data of second eluting diastereomer:

HPLC Rt_{H1}=2.409 min; ESIMS [M+H]⁺=374, 376 (1Br)

¹H-NMR (CDCl₃, 360 MHz): 7.46-7.38 (m, 1H), 7.36 (s, 1H), 7.31 (dd, 1H), 6.95 (dd, 1H), 6.23 (t, J=54 Hz, 1H), 4.53-4.44 (dd, 1H), 4.30-4.20 (m, 1H), 4.11-4.03 (m, 1H), 4.01-3.95 (dd, 1H), 1.77 (d, 3H).

### b) 5-(5-Bromo-2-fluoro-phenyl)-5-difluoromethyl-2-methyl-morpholin-3-one

A solution of N-[1-(5-bromo-2-fluoro-phenyl)-2,2-difluoro-1-hydroxymethyl-ethyl]-2-chloro-propionamide first eluting diastereomer (442 mg, 1.180 mmol) in 4.4 mL acetonitrile was treated with potassium hydroxide (86 mg, 1.298 mmol) and stirred over night. Additional potassium hydroxide (26 mg, 0.472 mmol) was added and the reaction mixture was stirred for another night. Eventually, the reaction mixture was partitioned between 1N HCl and EtOAc. The layers were separated, washed with brine and EtOAc. The combined organic layers were dried over MgSO₄.H₂O and evaporated. The crude product was crystallized from TBME to give 251 mg of the title compound as white crystals.

HPLC: Rt_{H1}=2.221 min; ESIMS [M+H]⁺=338, 340 (1Br);

¹H-NMR (DMSO-d6, 360 MHz): 8.96 (s, 1H), 7.82 (m, 1H), 7.73-7.65 (m, 1H), 7.30 (dd, 1H), 6.59 (t, J=54 Hz, 1H), 4.46 (d, 1H), 4.23-4.15 (dd, 1H), 3.89-3.80 (m, 1H), 1.33 (d, 3H).

### c) 5-(5-Bromo-2-fluoro-phenyl)-5-difluoromethyl-2-methyl-morpholine-3-thione

To a solution of 5-(5-bromo-2-fluoro-phenyl)-5-difluoromethyl-2-methyl-morpholin-3-one (659 mg, 1.949 mmol) in 6.6 mL pyridine was added phosphorus pentasulfide (433 mg, 1.949 mmol) and the mixture was heated to 80° C. for 120 minutes. The reaction mixture was cooled to room temperature and partitioned between 0.1 N NaOH and EtOAc. The layers were separated, washed with brine and EtOAc. The combined organic layers were dried over MgSO₄.H₂O and evaporated to give 704 mg of the title compound as a diastereomeric mixture.

HPLC: Rt_{H1}=2.961 min; ESIMS [M+H]⁺=354, 356 (1Br),
Rt_{H1}=3.007 min; ESIMS [M+H]⁺=354, 356 (1Br);

¹H-NMR of diastereomeric mixture (DMSO-d6, 360 MHz): 11.32 and 11.26 (s, 1H), 7.82-7.71 and 7.59 (m, 2H), 7.45-7.33 (m, 1H), 6.72 and 6.63 (t, J=54 Hz, 1H), 4.62-4.41 and 4.04-3.95 (m, 3H), 1.62 and 1.51 (d, 3H).

### d) [5-(5-Amino-2-fluoro-phenyl)-5-difluoromethyl-2-methyl-5,6-dihydro-2H-[1,4]oxazin-3-yl]-carbamic acid tert-butyl ester

This compound was obtained from 5-(5-bromo-2-fluoro-phenyl)-5-difluoromethyl-2-methyl-morpholine-3-thione by a similar sequence as described for example 42 steps g) to j) as a diastereomeric mixture (white foam).

HPLC: Rt_{H3}=2.793 min; ESIMS [M+H]⁺=374;

Rf (hexane/EtOAc 1/1): 0.40 (isomer 1, major spot), 0.47 (isomer 2, minor spot);

¹H-NMR of diastereomeric mixture (CDCl₃, 360 MHz, broad signals due to rotamers): 11.00 and 11.96 (s, 1H), 7.01-7.89 (m, 1H), 6.76-6.62 (m, 2H), 6.32 (t, J=54 Hz, 1H), 4.66-3.92 (m, 3H), 3.70 (s, 2H), 1.59-1.56 (s, 12H).

### e) (5-{5-[(5-Cyano-pyridine-2-carbonyl)-amino]-2-fluoro-phenyl}-5-difluoromethyl-2-methyl-5,6-dihydro-2H-[1,4]oxazin-3-yl)-carbamic acid tert-butyl ester

A solution of [5-(5-amino-2-fluoro-phenyl)-5-difluoromethyl-2-methyl-5,6-dihydro-2H-[1,4]oxazin-3-yl]-carbamic acid tert-butyl ester (140 mg, 0.375 mmol), 5-cyano-2-pyridinecarboxylic acid (83 mg, 0.562 mmol) and HOAT (92 mg, 0.675 mmol) in 2 mL DMF was cooled to 0-5° C. EDC (108 mg, 0.562 mmol) followed by DIPEA (97 mg, 0.750 mmol) was added. The reaction mixture was allowed to warm up to room temperature. After 135 minutes, the mixture was partitioned between saturated aqueous NaHCO₃ solution and EtOAc. The layers were separated, washed with saturated aqueous NaHCO₃ solution, brine and EtOAc. The combined organic layers were dried over MgSO₄.H₂O and evaporated. The crude product was purified on a silica gel column by eluting with hexane/EtOAc 3/1->2/1 to give the title compound as a diasteromeric mixture (white foam).

Rf (hexane/EtOAc 2/1): 0.36 (isomer 1), 0.30 (isomer 2);

HPLC: Rt_{H3}=2.870 min; ESIMS [M+H]⁺=504;

¹H-NMR of diastereomeric mixture (CDCl₃, 360 MHz, broad signals due to rotamers): 11.14 and 11.07 (s, 1H), 9.93 (s, 1H), 8.95 and 8.92 (s, 1H), 8.50-8.42 (m, 1H), 8.26 and 8.24 (d, 1H), 8.16-7.97 (m, 1H), 7.79-7.74 (m, 1H), 7.28-7.12 (m, 1H), 6.36 (t, J=54 Hz, 1H), 4.72-3.94 (m, 3H), 1.67-1.43 (m, 12H).

### f) 5-Cyano-pyridine-2-carboxylic acid [3-((3R*,6R*)-5-amino-3-difluoromethyl-6-methyl-3,6-dihydro-2H-[1,4]oxazin-3-yl)-4-fluoro-phenyl]-amide hydrochloride

To a solution of (5-{5-[(5-cyano-pyridine-2-carbonyl)-amino]-2-fluoro-phenyl}-5-difluoromethyl-2-methyl-5,6-dihydro-2H-[1,4]oxazin-3-yl)-carbamic acid tert-butyl ester (170 mg, 0.338 mmol) in 1.95 mL dichloromethane was added 0.65 mL TFA. After the solution had been stirring for 45 minutes it was evaporated at room temperature. The residue was taken up in EtOAc and extracted with saturated aqueous NaHCO₃ solution. The layers were separated, washed with brine and EtOAc. The combined organic layers were evaporated. The crude product was purified on a silica gel column by eluting with CH₂Cl₂/0.5-3% EtOH:NH₃ 9:1 to give a first and a second eluting isomer. Each isomer was individually dissolved in THF and 0.1 mL 1N HCl in diethyl ether was added. The mixtures were evaporated to give 35.8 mg of the first eluting and 43.5 mg of the second eluting isomer as their corresponding hydrochlorides.

Analytical data of first eluting isomer, 5-cyano-pyridine-2-carboxylic acid [3-((3R*,6R*)-5-amino-3-difluoromethyl-6-methyl-3,6-dihydro-2H-[1,4]oxazin-3-yl)-4-fluoro-phenyl]-amide hydrochloride:

HPLC: Rt_{H3}=2.774 min; ESIMS [M+H]⁺=404;

¹H-NMR (DMSO, 600 MHz): 11.06 (s, 1H), 10.90 (s, 1H), 9.57 (s, 1H), 9.22 (s, 1H), 8.85 (s, 1H), 8.61 (d, 1H), 8.30 (d, 1H), 8.17-8.13 (m, 1H), 8.09-8.04 (m, 1H), 7.41 (t, 1H), 6.81 (t, J=54 Hz, 1H), 4.81 (d, 1H), 4.46 (d, 1H), 4.04 (d, 1H), 1.57 (d, 3H).

### Example 76

### 5-Cyano-pyridine-2-carboxylic acid [3-((3R*,6S*)-5-amino-3-difluoromethyl-6-methyl-3,6-dihydro-2H-[1,4]oxazin-3-yl)-4-fluoro-phenyl]-amide hydrochloride

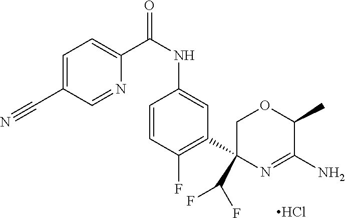 [SMILES: C[C@@H]1OC[C@](c2cc(NC(=O)c3ccc(C#N)cn3)ccc2F)(C(F)F)N=C1N ; Cl]

To a solution of (5-{5-[(5-cyano-pyridine-2-carbonyl)-amino]-2-fluoro-phenyl}-5-difluoromethyl-2-methyl-5,6-dihydro-2H-[1,4]oxazin-3-yl)-carbamic acid tert-butyl ester [example 75 step e)] (170 mg, 0.338 mmol) in 1.95 mL dichloromethane was added 0.65 mL TFA. After the solution had been stirring for 45 minutes it was evaporated at room temperature. The residue was taken up in EtOAc and extracted with saturated aqueous NaHCO₃ solution. The layers were separated, washed with brine and EtOAc. The combined organic layers were evaporated. The crude product was purified on a silica gel column by eluting with CH2Cl2/0.5-3% EtOH:NH₃ 9:1 to give a first and a second eluting isomer. Each isomer was individually dissolved in THF and 0.1 mL 1N HCl in diethyl ether was added. The mixtures were evaporated to give 35.8 mg of the first eluting and 43.5 mg of the second eluting isomer as their corresponding hydrochlorides.

Analytical data of second eluting isomer, 5-cyano-pyridine-2-carboxylic acid [3-((3R*,6S*)-5-amino-3-difluoromethyl-6-methyl-3,6-dihydro-2H-[1,4]oxazin-3-yl)-4-fluoro-phenyl]-amide hydrochloride:

HPLC: Rt_{H3}=2.746 min; ESIMS [M+H]⁺=404;

¹H-NMR (DMSO, 600 MHz): 11.17 (s, 1H), 11.05 (s, 1H), 9.71 (s, 1H), 9.22 (s, 1H), 8.92 (s, 1H), 8.60 (d, 1H), 8.31 (d, 1H), 8.16-8.10 (m, 1H), 8.08-8.02 (m, 1H), 7.40 (t, 1H), 6.77 (t, J=54 Hz, 1H), 4.86 (d, 1H), 4.34 (d, 1H), 4.13 (d, 1H), 1.50 (d, 3H).

### Example 77

### 5-Bromo-pyridine-2-carboxylic acid [3-((3R*,6R*)-5-amino-3-difluoromethyl-6-methyl-3,6-dihydro-2H-[1,4]oxazin-3-yl)-4-fluoro-phenyl]-amide hydrochloride

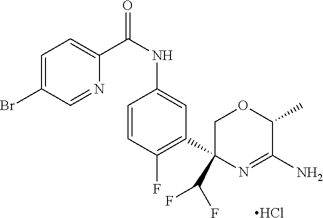 [SMILES: C[C@H]1OC[C@](c2cc(NC(=O)c3ccc(Br)cn3)ccc2F)(C(F)F)N=C1N ; Cl]

### a) (5-{5-[(5-Bromo-pyridine-2-carbonyl)-amino]-2-fluoro-phenyl}-5-difluoromethyl-2-methyl-5,6-dihydro-2H-[1,4]oxazin-3-yl)-carbamic acid tert-butyl ester

This compound was prepared from 5-(5-amino-2-fluoro-phenyl)-5-difluoromethyl-2-methyl-5,6-dihydro-2H-[1,4]oxazin-3-yl]-carbamic acid tert-butyl ester [example 75 step d)] in an analogous manner as described for example 75 step e).

Rf (hexane/EtOAc 3/1): 0.26 (isomer 1), 0.22 (isomer 2);

HPLC: Rt_{H3}=3.137 min; ESIMS [M+H]⁺=557/559;

¹H-NMR of diastereomeric mixture (CDCl₃, 360 MHz, broad signals due to rotamers): 11.04 and 10.97 (s, 1H), 9.79 (s, 1H), 8.63-8.57 (m, 1H), 8.13-8.06 (m, 1H), 8.03-7.45 (m, 3H), 7.16-7.00 (m, 1H), 6.24 (t, J=54 Hz, 1H), 4.62-3.86 (m, 3H), 1.56-1.34 (m, 12H).

### b) 5-Bromo-pyridine-2-carboxylic acid[3-((3R*,6R*)-5-amino-3-difluoromethyl-6-methyl-3,6-dihydro-2H-[1,4]oxazin-3-yl)-4-fluoro-phenyl]-amide hydrochloride

(5-{5-[(5-Bromo-pyridine-2-carbonyl)-amino]-2-fluoro-phenyl}-5-difluoromethyl-2-methyl-5,6-dihydro-2H-[1,4]oxazin-3-yl)-carbamic acid tert-butyl ester (179 mg, 0.321 mmol) was dissolved in HCl solution 4 mol/L in dioxane (2.4 mL, 9.63 mmol) and 0.1 mL HCl solution 3 mol/L in methanol was added as a co-solvent. The sealed reaction vessel was heated to 50° C. for 120 minutes. The mixture was evaporated; its residue was taken up in EtOAc and extracted with saturated aqueous NaHCO₃ solution. The layers were separated, washed with brine and EtOAc. The combined organic layers were evaporated. The crude product was purified on a silica gel column by eluting with CH₂Cl₂/0.5-2% EtOH:NH₃ 9:1 to give a first and a second eluting isomer. Each isomer was individually dissolved in THF and 0.1 mL 1N HCl in diethyl ether was added. The mixtures were evaporated to give 37.0 mg of the first eluting and 54.3 mg of the second eluting isomer as their corresponding hydrochlorides.

Analytical data of first eluting isomer, 5-bromo-pyridine-2-carboxylic acid [3-((3R*,6R*)-5-amino-3-difluoromethyl-6-methyl-3,6-dihydro-2H-[1,4]oxazin-3-yl)-4-fluoro-phenyl]-amide hydrochloride:

HPLC: Rt_{H3}=2.910 min; ESIMS [M+H]⁺=457/459;

¹H-NMR (DMSO, 600 MHz): 10.93 (s, 1H), 10.91 (s, 1H), 9.63 (s, 1H), 8.94 (s, 1H), 8.88 (s, 1H), 8.35 (d, 1H), 8.15-8.04 (m, 3H), 7.39 (dd, 1H), 6.81 (t, J=54 Hz, 1H), 4.81 (d, 1H), 4.48 (d, 1H), 4.05 (d, 1H), 1.58 (d, 3H).

### Example 78

### 5-Bromo-pyridine-2-carboxylic acid [3-((3R*,6S*)-5-amino-3-difluoromethyl-6-methyl-3,6-dihydro-2H-[1,4]oxazin-3-yl)-4-fluoro-phenyl]-amide hydrochloride

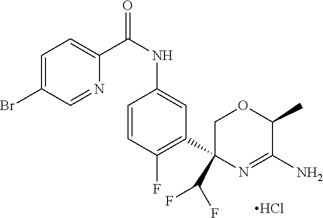 [SMILES: C[C@@H]1OC[C@](c2cc(NC(=O)c3ccc(Br)cn3)ccc2F)(C(F)F)N=C1N ; Cl]

(5-{5-[(5-Bromo-pyridine-2-carbonyl)-amino]-2-fluoro-phenyl}-5-difluoromethyl-2-methyl-5,6-dihydro-2H-[1,4]oxazin-3-yl)-carbamic acid tert-butyl ester [example 77 step a)] (179 mg, 0.321 mmol) was dissolved in HCl solution 4 mol/L in dioxane (2.4 mL, 9.63 mmol) and 0.1 mL HCl solution 3 mol/L in methanol was added as a co-solvent. The sealed reaction vessel was heated to 50° C. for 120 minutes. The mixture was evaporated; its residue was taken up in EtOAc and extracted with saturated aqueous NaHCO₃ solution. The layers were separated, washed with brine and EtOAc. The combined organic layers were evaporated. The crude product was purified on a silica gel column by eluting with CH₂Cl₂/0.5-2% EtOH:NH₃ 9:1 to give a first and a second eluting isomer. Each isomer was individually dissolved in THF and 0.1 mL 1N HCl in diethyl ether was added. The mixtures were evaporated to give 37.0 mg of the first eluting and 54.3 mg of the second eluting isomer as their corresponding hydrochlorides.

Analytical data of second eluting isomer, 5-bromo-pyridine-2-carboxylic acid [3-((3R*,6S*)-5-amino-3-difluoromethyl-6-methyl-3,6-dihydro-2H-[1,4]oxazin-3-yl)-4-fluoro-phenyl]-amide hydrochloride:

HPLC: Rt_{H3}=2.916 min; ESIMS [M+H]⁺=457/459;

¹H-NMR (DMSO, 600 MHz): 11.03 (s, 2H), 9.70 (s, 1H), 8.91 (s, 1H), 8.88 (s, 1H), 8.34 (d, 1H), 8.13-8.08 (m, 2H), 8.04-8.00 (m, 1H), 7.38 (dd, 1H), 6.77 (t, J=54 Hz, 1H), 4.86 (d, 1H), 4.34 (d, 1H), 4.13 (d, 1H), 1.50 (d, 3H).

### Example 79

### 5-Cyano-pyridine-2-carboxylic acid[3-(5-amino-3-difluoromethyl-6,6-dimethyl-3,6-dihydro-2H-[1,4]oxazin-3-yl)-4-fluoro-phenyl]-amide

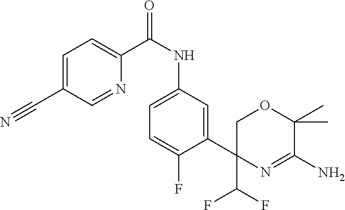 [SMILES: CC1(C)OCC(c2cc(NC(=O)c3ccc(C#N)cn3)ccc2F)(C(F)F)N=C1N]

### a) 2-(5-Bromo-2-fluoro-phenyl)-2-difluoromethyl-1-(2-nitro-benzenesulfonyl)-aziridine

2-Amino-2-(5-bromo-2-fluoro-phenyl)-3,3-difluoro-propan-1-ol (13.04 g, 45.9 mmol) [examples 42 step d)] was dissolved in 261 mL acetonitrile, 2-nitrobenzenesulfonyl chloride (22.38 g, 101 mmol) and potassium hydrogencarbonate (13.79 g, 138 mmol) were added. The mixture was heated to 80° C. and stirred over night. After this period, the reaction mixture was cooled down and partitioned between saturated aqueous NaHCO₃ solution and TBME. The layers were separated, washed with brine and TBME. The combined organic layers were dried over MgSO₄.H₂O and evaporated. The crude product was purified on a silica gel column by eluting with hexane/dichloromethane 2/1->1/2 to give 7.71 g of the title compound as white crystals.

HPLC: Rt_{H1}=3.309 min; ESIMS [M+Na]⁺=473, 475 (1Br);

¹H-NMR (CDCl₃, 360 MHz): 8.33-8.26 (m, 1H), 7.87-7.78 (m, 3H), 7.76 (dd, 1H), 7.60-7.53 (m, 1H), 7.05 (t, 1H), 6.22 (t, J=54 Hz, 1H), 3.42 (s, 1H), 3.28 (s, 1H).

### b) 2-[2-(5-Bromo-2-fluoro-phenyl)-3,3-difluoro-2-(2-nitro-benzenesulfonylamino)-propoxy]-2-methyl-propionic acid tert-butyl ester

To a solution of tert-butyl α-hydroxyisobutyrate (533 mg, 3.32 mmol) in 4.5 mL DMF and 0.75 mL THF was added portion wise (133 mg, 3.32 mmol) sodium hydride at room temperature. After the reaction mixture had been stirring for 15 minutes, a solution of 2-(5-bromo-2-fluoro-phenyl)-2-difluoromethyl-1-(2-nitro-benzenesulfonyl)-aziridine (1 g, 2.22 mmol) was added. The reaction mixture was stirred at rt for 150 minutes and quenched with aqueous NH₄Cl solution. TBME was added, the layers were separated, washed with brine and TBME. The combined organic layers were dried over MgSO₄.H₂O and evaporated. The crude product was purified on a silica gel column by eluting with hexane/EtOAc 6/1->5/1 to give 1.10 g of the title compound as a pale yellow resin.

HPLC: Rt_{H7}=3.471 min; ESIMS [M+Na]⁺=633, 635 (1Br);

¹H-NMR (CDCl₃, 360 MHz): 7.94 (dd, 1H), 7.82 (dd, 1H), 7.68 (t, 1H), 7.57 (d, 1H), 7.49 (t, 1H), 7.42 (s, 1H), 7.37-7.32 (m, 1H), 6.90 (dd, 1H), 6.72 (t, J=54 Hz, 1H), 4.02 (d, 1H), 3.94 (d, 1H), 1.58 (s, 9H), 1.47 (s, 3H), 1.45 (s, 3H).

### c) 5-(5-Bromo-2-fluoro-phenyl)-5-difluoromethyl-2,2-dimethyl-4-(2-nitro-benzenesulfonyl)-morpholin-3-one

To a solution of 2-[2-(5-bromo-2-fluoro-phenyl)-3,3-difluoro-2-(2-nitro-benzenesulfonylamino)-propoxy]-2-methyl-propionic acid tert-butyl ester (1.10 g, 1.80 mmol) in 8 mL dichloromethane was added 4 mL trifluoroacetic acid. After the reaction mixture had been stirring at room temperature for 60 minutes, it was evaporated to give 1.10 g of a white solid. This solid was directly dissolved in a mixture of 10 mL dichloromethane and N-methylmorpholine (546 mg, 5.40 mmol) followed by drop wise addition of ethyl chloroformate (293 mg, 2.70 mmol). After the reaction mixture had been stirring for 150 minutes at room temperature, the reaction mixture was partitioned between TBME and saturated aqueous NaHCO₃. The layers were separated, washed with 1N HCl, brine and TBME. The combined organic layers were dried over MgSO₄.H₂O and evaporated. The crude product was crystallized from TBME/hexane to give 822 mg of the title compound as white crystals.

HPLC: Rt_{H6}=3.087 min; ESIMS [M+H]⁺=537, 539 (1Br);

¹H-NMR (CDCl₃, 360 MHz): 8.15 (d, 1H), 7.82-7.70 (m, 2H), 7.65 (dd, 1H), 7.61-7.53 (m, 2H), 7.18 (t, J=54 Hz, 1H), 7.09 (dd, 1H), 4.49 (d, 1H), 4.25 (d, 1H), 1.56 (s, 3H), 1.40 (s, 3H)

### d) 5-(5-Bromo-2-fluoro-phenyl)-5-difluoromethyl-2,2-dimethyl-morpholin-3-one

To a solution of 5-(5-bromo-2-fluoro-phenyl)-5-difluoromethyl-2,2-dimethyl-4-(2-nitro-benzenesulfonyl)-morpholin-3-one (6.29 g, 11.71 mmol) and thioglycolic acid (1.83 g, 19.90 mmol) in 63 mL DMF was added potassium carbonate (6.47 g, 46.8 mmol). The reaction mixture was heated to 60° C. After 120 minutes, additional thioglycolic acid (324 mg, 3.51 mmol) was added. 30 minutes later on, the reaction mixture was cooled to room temperature and partitioned between EtOAc and water. The layers were separated, washed with saturated aqueous NaHCO₃, brine and EtOAc. The combined organic layers were dried over MgSO₄.H₂O and evaporated. The crude product was crystallized from TBME/hexane to give 3.14 g of the title compound as white crystals.

HPLC: Rt_{H1}=2.476 min; ESIMS [M+H]⁺=352, 354 (1Br);

¹H-NMR (DMSO-d6, 360 MHz): 8.94 (s, 1H), 7.76-7.65 (m, 2H), 7.32 (dd, 1H), 6.55 (t, J=54 Hz, 1H), 4.20 (d, 1H), 4.08 (d, 1H), 1.37 (s, 3H), 1.28 (s, 3H).

### e) 5-Difluoromethyl-5-(2-fluoro-phenyl)-2,2-dimethyl-morpholin-3-one

5-(5-Bromo-2-fluoro-phenyl)-5-difluoromethyl-2,2-dimethyl-morpholin-3-one (3.14 g, 8.92 mmol) and sodium acetate (1.46 g, 17.83 mmol) were suspended in 100 mL methanol and 10 mL THF. Eventually, 10% Pd on charcoal (315 mg) was added and the reaction mixture was treated with hydrogen (balloon) at rt. After 60 minutes the reaction mixture was filtered over celite and evaporated. The residue was partitioned between aqueous Na₂CO₃ solution and EtOAc. The layers were separated, washed with brine and EtOAc. The combined organic layers were dried over MgSO₄.H₂O and evaporated to give 2.42 g of the title compound as a white solid.

HPLC: Rt_{H3}=3.008 min; ESIMS [M+H]⁺=274;

¹H-NMR (DMSO-d6, 360 MHz): 8.87 (s, 1H), 7.56 (t, 1H), 7.53-7.45 (m, 1H), 7.36-7.24 (m, 2H), 6.54 (t, J=54 Hz, 1H), 4.19 (d, 1H), 4.09 (d, 1H), 1.37 (s, 3H), 1.27 (s, 1H).

### f) 5-Difluoromethyl-5-(2-fluoro-phenyl)-2,2-dimethyl-morpholine-3-thione

To a solution of 5-difluoromethyl-5-(2-fluoro-phenyl)-2,2-dimethyl-morpholin-3-one (2.41 g, 8.82 mmol) and hexamethyldisiloxane (2.58 g, 15.88 mmol) in toluene was added) phosphorous pentasulfide (2.35 g, 10.58 mmol). The reaction mixture was heated to 100° C. and stirred over night. After the reaction mixture had been cooled to room temperature, 23 mL Acetone and 33 mL aqueous K₂CO₃ solution (10% w/w) were added. This mixture was stirred for 90 minutes and then partitioned between water and EtOAc. The layers were separated, washed with 0.1 N NaOH, brine and EtOAc. The organic layers were combined, dried over MgSO₄.H₂O and evaporated. The crude product was crystallized from TBME/hexane to give 2.28 g of the title compound as white crystals

HPLC: Rt_{H3}=3.503 min; ESIMS [M+H]⁺=290;

¹H-NMR (DMSO-d6, 360 MHz): 11.13 (s, 1H), 7.55-7.42 (m, 2H), 7.37-7.28 (m, 2H), 6.61 (t, J=54 Hz, 1H), 4.19 (dd, 2H), 1.60 (s, 3H), 1.48 (s, 3H).

### g) 5-Difluoromethyl-5-(2-fluoro-phenyl)-2,2-dimethyl-5,6-dihydro-2H-[1,4]oxazin-3-ylamine

5-Difluoromethyl-5-(2-fluoro-phenyl)-2,2-dimethyl-morpholine-3-thione (2.50 g, 8.64 mmol) was dissolved in NH₃ solution 7 mol/L in methanol (40.7 mL, 285 mmol). The sealed reaction vessel was heated to 80° C. for 7 h, then the temperature was lowered to 70° C. and the reaction mixture was stirred over night. The reaction mixture was evaporated and purified on a silica gel column by eluting with CH₂Cl₂/1-4% EtOH:NH₃ 9:1 to give 2.09 g of the title compound as an off-white solid.

HPLC: Rt_{H3}=2.575 min; ESIMS [M+H]⁺=273;

¹H-NMR (DMSO-d6, 360 MHz): 7.78 (t, 1H), 7.41-7.32 (m, 1H), 7.26-7.11 (m, 2H), 6.14 (s, 2H), 6.11 (t, J=54 Hz, 1H), 4.11 (dd, 1H), 3.87 (d, 1H), 1.39 (s, 3H), 1.24 (s, 3H).

### h) 5-Cyano-pyridine-2-carboxylic acid[3-(5-amino-3-difluoromethyl-6,6-dimethyl-3,6-dihydro-2H-[1,4]oxazin-3-yl)-4-fluoro-phenyl]-amide

This compound was obtained from 5-difluoromethyl-5-(2-fluoro-phenyl)-2,2-dimethyl-5,6-dihydro-2H-[1,4]oxazin-3-ylamine by a similar sequence as described for example 98 steps h) to l). With the exception that after the extraction, the base was not converted into a hydrochloride. The free base was crystallized from 2-propanol instead to give the title compound as white crystals.

HPLC: Rt_{H3}=2.818 min; ESIMS [M+H]⁺=418;

¹H-NMR (DMSO, 600 MHz): 10.84 (s, 1H), 9.20 (s, 1H) 8.58 (d, 1H), 8.28 (d, 1H), 8.14-8.10 (m, 1H), 7.85-7.80 (m, 1H), 7.18 (t, 1H), 6.13 (s, 2H), 6.13 (t, J=54 Hz, 1H), 4.04 (d, 1H), 3.87 (d, 1H), 1.38 (s, 3H), 1.26 (s, 3H).

### Example 80

The compounds listed in Table 10 can be prepared by a procedure analogous to that used in example 79, using 4N HCl in dioxane in the last step.

TABLE 10

| | | | MS |
|---|---|---|---|
| | | ¹H-NMR | [m/z; |
| Example | Compound | (δ; DMSO-d₆) | (M + 1)⁺] |
| 80 | 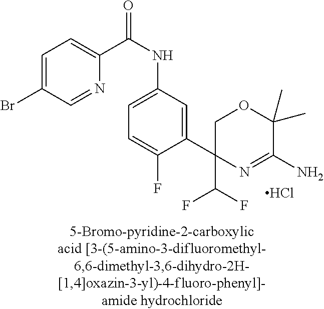 [SMILES: CC1(C)OCC(c2cc(NC(=O)c3ccc(Br)cn3)ccc2F)(C(F)F)N=C1N ; Cl] | 11.01 (s, 2H), 9.76 (s, 1H) 8.97 (s, 1H), 8.88 (s, 1H), 8.35 (d, 1H), 8.10 (d, 2H), 8.06 (d, 1H), 7.39 (t, 1H), 6.79 (t, J = 54 Hz, 1H), 4.21 (dd, 2H), 1.61 (s, 3H), 1.55 (s, 3H) | 471, 473 |

### Examples 81 to 84

The compounds listed in Table 11 can be prepared by a procedure analogous to that described in example 34, starting from 1,5-dibromo-2,4-difluoro-benzene.

TABLE 11

| | | | MS |
|---|---|---|---|
| | | ¹H-NMR | [m/z; |
| Example | Compound | (δ; DMSO-d₆) | (M + 1)⁺] |
| 81 | 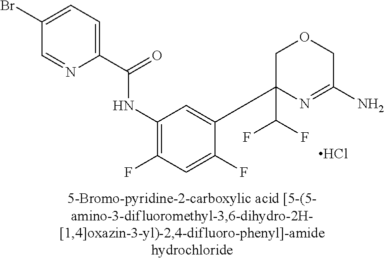 [SMILES: NC1=NC(c2cc(NC(=O)c3ccc(Br)cn3)c(F)cc2F)(C(F)F)COC1 ; Cl] | 10.45 (s, 1 H), 8.88 (d, 1 H), 8.33 (dd, 1 H), 8.16 (t, 1 H), 8.06 (d, 1 H), 7.37 (t, 1 H), 6.21 (br. s., 2 H), 6.09 (t, 1 H, CHF2), 4.19 (dd, 1 H), 4.02 (d, 1 H), 3.91 (d, 1 H), 3.80 (d, 1 H) | 461, 463 |
| 82 | 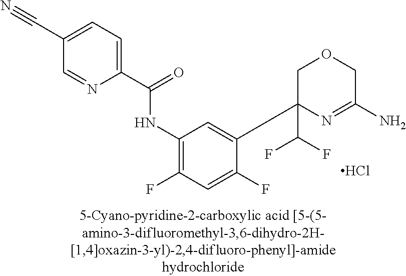 [SMILES: N#Cc1ccc(C(=O)Nc2cc(C3(C(F)F)COCC(N)=N3)c(F)cc2F)nc1 ; Cl] | 11.06 (s, 1 H), 10.76 (s, 1 H), 9.76 (s, 1 H), 9.24 (s, 1 H), 8.78 (s, 1 H), 8.61 (dd, 1 H), 8.28 (d, 1 H), 7.94 (t, 1 H), 7.66 (t, 1 H), 6.76 (t, 1 H, CHF2), 4.77- 4.58 (m, 2 H), 4.36 (d, 1 H), 4.21 (d, 1 H) | 408 |
| 83 | 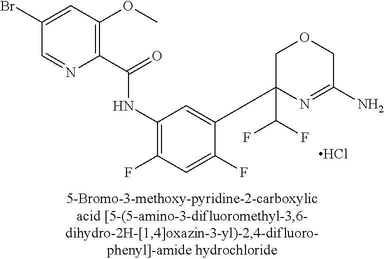 [SMILES: COc1cc(Br)cnc1C(=O)Nc1cc(C2(C(F)F)COCC(N)=N2)c(F)cc1F ; Cl] | 11.07 (s, 1 H), 10.37 (s, 1 H), 9.77 (s, 1 H), 8.78 (s, 1 H), 8.37 (s, 1 H), 8.06-7.98 (m, 2 H), 7.62 (t, 1 H), 6.76 (t, 1 H, CHF2), 4.76- 4.59 (m, 2 H), 4.37 (d, 1 H), 4.19 (d, 1 H), 3.91 (s, 3 H) | 491, 493 |
| 84 | 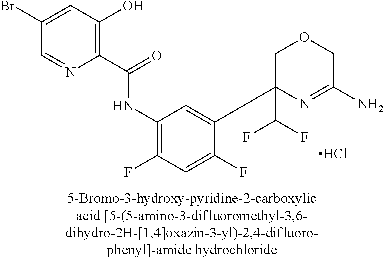 [SMILES: NC1=NC(c2cc(NC(=O)c3ncc(Br)cc3O)c(F)cc2F)(C(F)F)COC1 ; Cl] | 11.99 (br. s., 1 H), 11.03 (br. s., 1 H), 10.96 (br. s., 1 H), 9.74 (br. s., 1 H), 8.76 (br. s., 1 H), 8.39 (s, 1 H), 7.91 (s, 1 H), 7.82 (br. s., 1 H), 7.66 (br. s., 1 H), 6.75 (t, 1 H, CHF2), 4.70- 4.61 (m, 2 H), 4.36 (d, 1 H), 4.20 (d, 1 H) | 477, 479 |

### Example 85

### 5-Chloro-pyridine-2-carboxylic acid [3-((R)-5-amino-3-fluoromethyl-3,6-dihydro-2H-[1,4]oxazin-3-yl)-4-fluoro-phenyl]-amide hydrochloride

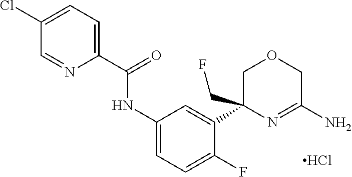 [SMILES: NC1=N[C@](CF)(c2cc(NC(=O)c3ccc(Cl)cn3)ccc2F)COC1 ; Cl]

### a) 4-Bromo-1-fluoro-2-nitromethyl-benzene

A mixture of 4-bromo-1-fluoro-2-bromomethyl-benzene (5 g, 18.66 mmol) and AgNO₂ (3.45 g, 22.39 mmol) were stirred in 62 ml TBME for 7 h. The dark mixture was filtered over celite, washed with TBME and evaporated. The crude product was purified by chromatography on silica gel (heptane/EtOAc 20/1) to provide the title compound as a yellow oil.

TLC (Hex: EE/9:1) Rf 0.3

HPLC: Rt_{H4}=2.449 min;

¹H-NMR (CDCl₃, 360 MHz): 7.64-7.58 (m, 2H), 7.12 (t, 1H), 5.50 (s, 2H).

### b) 2-(5-Bromo-2-fluoro-phenyl)-2-nitro-propane-1,3-diol

A solution of 4-bromo-1-fluoro-2-nitromethyl-benzene (7.75 g, 33.1 mmol), formaldehyde (35%, aqueous) (5.47 ml, 69.5 mmol) and Et₃N (2.3 ml, 16.56 mmol) were stirred in 66 ml dioxane for 3 h. The solution was diluted with brine and extracted with TBME. The organic layer was washed with brine, dried with Na₂SO₄ and evaporated. The crude product was purified by chromatography on silica gel (heptane/EtOAc 3/1) to provide the title compound as a white solid.

TLC (Hex: EE/2:1) Rf 0.24

HPLC: Rt_{H4}=2.070 min; ESIMS [M+Na]⁺=316, 318 (1Br);

¹H-NMR (DMSO, 360 MHz): 7.65-7.60 (m, 1H), 7.55 (dd, 1H), 7.75 (dd, 1H), 5.50 (s, 2H), 4.20 (br t, 4H).

### c) 2-Amino-2-(5-bromo-2-fluoro-phenyl)-propane-1,3-diol

A solution of 2-(5-bromo-2-fluoro-phenyl)-2-nitro-propane-1,3-diol (7 g, 23.8 mmol) in 35 ml AcOH was added dropwise to a mixture of zinc (9.34 g, 143 mmol) in 35 ml AcOH while the temperature did not rise above 40° C. The mixture was stirred for 1 h, filtered over celite and washed with MeOH. The filtrate was evaporated, diluted with water and washed with TBME. The aqueous layer was basified with 2 N NaOH and NH₃ (25%, aqueous), saturated with NaCl and extracted with EtOAc. The organic layer was washed with brine, dried with Na₂SO₄ and evaporated to provide the title compound as an off-white solid.

TLC (EE: MeOH/19:1+1% NH3 (25%, aqueous)) Rf 0.38

HPLC: Rt_{H2}=2.332 min; ESIMS [M+H]⁺=246, 266 (1Br);

¹H-NMR (DMSO, 360 MHz): 7.82 (dd, 1H), 7.50-7.42 (m, 1H), 7.09 (dd, 1H), 4.71 (br s, 2H), 3.36 (dd, 4H), 2.20 (br s, 2H).

### d) N-[1-(5-Bromo-2-fluoro-phenyl)-2-hydroxy-1-hydroxymethyl-ethyl]-2-chloro-acetamide

A solution of chloro-acetyl chloride (6.39 ml, 80 mmol) in 10 ml ACN was added dropwise to a mixture of 2-amino-2-(5-bromo-2-fluoro-phenyl)-propane-1,3-diol (5.3 g, 20 mmol) and K₂CO₃ (11.1 g, 80 mmol) in 90 ml ACN while the temperature did not rise above 35° C. The mixture was stirred for 2 h. MeOH (40 ml, 99 mmol) were added and after 5 min stirring the mixture was filtered over celite and washed with MeOH. The filtrate was acidified with citric acid solution (10%, aqueous) (pH 4-5) and partly evaporated. The remaining aqueous layer was extracted with EtOAc. The organic layer was washed with NaHCO₃ solution (10%, aqueous) and brine, dried with Na₂SO₄ and evaporated to provide the title compound as an off-white solid.

TLC (Hex: EE/1:1) Rf 0.23

HPLC: Rt_{H4}=1.966 min; ESIMS [M+H]⁺=340, 342 (1Br);

¹H-NMR (DMSO, 360 MHz): 8.19 (s, 1H), 7.47 (dd, 1H), 7.10 (dd, 1H), 5.00 (t, 2H), 4.19 (s, 2H), 3.98-3.81 (m, 4H).

### e) 5-(5-Bromo-2-fluoro-phenyl)-5-hydroxymethyl-morpholin-3-one

A mixture of N-[1-(5-bromo-2-fluoro-phenyl)-2-hydroxy-1-ydroxymethyl-ethyl]-2-chloro-acetamide (6.34 g, 18.62 mmol) and potassium tert.-butoxide (2.09 g, 18.62 mmol) in 62 ml t-BuOH was refluxed for 30 min. 19 ml 1 N HCl and water were added and the aqueous layer was extracted with EtOAc. The organic layer was washed with brine, dried with MgSO₄ and evaporated. The crude product was recrystallized in Hex/EtOAc to provide the title compound as an off-white solid.

TLC (Hex: EE/1:2) Rf 0.25

HPLC: Rt_{H4}=1.885 min; ESIMS [M+H]⁺=304, 306 (1Br);

¹H-NMR (DMSO, 360 MHz): 8.49 (s, 1H), 7.62-7.56 (m, 2H), 7.21 (dd, 1H), 5.25 (t, 1H), 4.15 (d, 1H), 4.02 (s, 2H), 3.91 (d, 1H), 3.79-3.62 (m, 2H).

### f) 5-(5-Bromo-2-fluoro-phenyl)-5-fluoromethyl-morpholin-3-one

To a solution of 5-(5-bromo-2-fluoro-phenyl)-5-hydroxymethyl-morpholin-3-one (1.6 g, 5.26 mmol) in 30 ml THF was added dropwise diethylaminosulfur trifluoride (0.97 ml, 7.34 mmol) and stirred for 2 h. The colorless solution was slowly added to an ice cooled Na₂CO₃ solution (10%, aqueous) and extracted with TBME. The organic layer was washed with brine, dried with MgSO₄ and evaporated. The crude product was purified by chromatography on silica gel (heptane/EtOAc 3/1) to provide the title compound as a slightly yellow solid.

TLC (Hex: EE/1:1) Rf 0.43

HPLC: Rt_{H4}=2.136 min; ESIMS [M+H]⁺=306, 308 (1Br);

¹H-NMR (CDCl₃, 360 MHz): 7.50-7.40 (m, 2H), 6.95 (dd, 1H), 6.55 (s, 1H), 4.86-4.58 (m, 2H), 4.22-4.11 (m, 2H), 4.07-3.98 (m, 2H).

### g) [5-(5-Amino-2-fluoro-phenyl)-5-fluoromethyl-5,6-dihydro-2H-[1,4]oxazin-3-yl]-carbamic acid tert-butyl ester

This compound was obtained from 5-(5-bromo-2-fluoro-phenyl)-5-fluoromethyl-morpholin-3-one by a similar sequence as described for example 42 steps g) to j).

TLC (Hex: EE/1:1) Rf 0.38

HPLC: Rt_{H2}=2.778 min; ESIMS [M+H]⁺=342;

¹H-NMR (DMSO, 360 MHz, broad signals due to rotamers): 9.79 (s, 1H), 6.82 (br t, 1H), 6.70-6.62 (m, 1H), 6.51-6.43 (m, 1H), 4.92 (s, 2H), 4.70-4.38 (m, 4H), 3.95-3.81 (m, 2H), 1.43 (s, 9H).

### h) [(R)-5-(5-Amino-2-fluoro-phenyl)-5-fluoromethyl-5,6-dihydro-2H-[1,4]oxazin-3-yl]-carbamic acid tert-butyl ester

The racemic product [5-(5-amino-2-fluoro-phenyl)-5-fluoromethyl-5,6-dihydro-2H-[1,4]oxazin-3-yl]-carbamic acid tert-butyl ester was separated via prep-HPLC on Chiralpak AD 20 μm 5×50×100 mm (5×SMB columns) (Flowrate: 65 ml/min; Detection UV: 220 nm). The desired compound was the slower eluting (R)-enantiomer.

Purity: 99.0% ee

[α]_{D}=−140° (c=1, CHCl₃).

### i) ((R)-5-{5-[(5-Chloro-pyridine-2-carbonyl)-amino]-2-fluoro-phenyl}-5-fluoromethyl-5,6-dihydro-2H-[1,4]oxazin-3-yl)-carbamic acid tert-butyl ester

[(R)-5-(5-Amino-2-fluoro-phenyl)-5-fluoromethyl-5,6-dihydro-2H-[1,4]oxazin-3-yl]-carbamic acid tert-butyl ester (75 mg, 0.22 mmol), 5-chloro-pyridine-2-carboxylic acid (38.1 mg, 0.242 mmol), HOAt (38.9 mg, 0.286 mmol), EDC (63.2 mg, 0.33 mmol) and Et₃N (77 μl, 0.549 mmol) were dissolved in CH₂Cl₂ and stirred for 14 h. The solution was evaporated and the crude product was purified by chromatography on silica gel (heptane/EtOAc 6/1) to provide the title compound as a white solid.

TLC (Hex: EE/2:1) Rf 0.46

HPLC: Rt_{H1}=2.668 min; ESIMS [M+H]⁺=481, 483 (1 Cl);

¹H-NMR (DMSO, 360 MHz, broad signals due to rotamers): 9.79 (br s, 1H), 8.50 (s, 1H), 8.29 (d, 1H), 7.90-7.85 (m, 1H), 7.81 (dd, 1H), 7.65-7.55 (br m, 1H), 7.10-7.00 (m, 1H), 4.75-4.45 (br m, 4H), 4.19 (d, 1H), 3.91-3.81 (1H), 1.46 (br s, 9H).

### j) 5-Chloro-pyridine-2-carboxylic acid [3-((R)-5-amino-3-fluoromethyl-3,6-dihydro-2H-[1,4]oxazin-3-yl)-4-fluoro-phenyl]-amide hydrochloride

A solution of ((R)-5-{5-[(5-chloro-pyridine-2-carbonyl)-amino]-2-fluoro-phenyl}-5-fluoromethyl-5,6-dihydro-2H-[1,4]oxazin-3-yl)-carbamic acid tert-butyl ester (106 mg, 0.22 mmol), 4 N HCl/Dioxan (1.1 ml, 4.41 mmol) and 3 N HCl/MeOH in CH₂Cl₂ was stirred for 15 h at room temperature and for 2 h at 40° C. to complete the conversion. The yellow solution was evaporated and taken up in MeOH. TBME was added and the white precipitate was filtered off to provide the title compound.

HPLC: Rt_{H2}=3.033 min; ESIMS [M+H]⁺=381, 383 (1Cl);

¹H-NMR (DMSO, 360 MHz): 11.90 (s, 1H), 11.85 (br s, 1H), 9.50 (br s, 1H), 8.83-8.80 (m, 1H), 8.25-8.15 (m, 2H), 8.10-8.01 (m, 2H), 7.40-7.32 (m, 2H), 5.08-4.97 (m, 1H), 4.95-4.82 (m, 1H), 4.71-4.60 (m, 1H), 4.20-4.10 (m, 1H).

### Examples 86 to 95

The compounds listed in Table 12 can be prepared by a procedure analogous to that used in example 85, using the racemic [5-(5-amino-2-fluoro-phenyl)-5-fluoromethyl-5,6-dihydro-2H-[1,4]oxazin-3-yl]-carbamic acid tert-butyl ester [example 85 step g)] or [(R)-5-(5-amino-2-fluoro-phenyl)-5-fluoromethyl-5,6-dihydro-2H-[1,4]oxazin-3-yl]-carbamic acid tert-butyl ester [example 85 step g)].

TABLE 12

| | | | MS |
|---|---|---|---|
| | | ¹H-NMR | [m/z; |
| Example | Compound | (δ; DMSO-d₆) | (M + 1)⁺] |
| 86 | 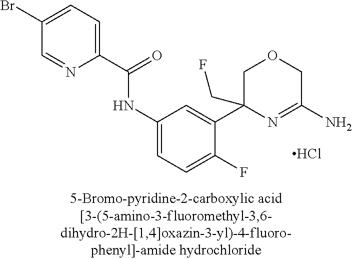 [SMILES: NC1=NC(CF)(c2cc(NC(=O)c3ccc(Br)cn3)ccc2F)COC1 ; Cl] | 10.95-10.85 (m, 2H), 9.62-9.51 (m, 1H), 8.89 (s, 1H), 8.80-8.75 (m, 1H), 8.35 (dd, 1H), 8.30 (d, 1H), 8.08-8.05 (m, 1H), 8.04-8.02 (m, 1H), 7.37-7.31 (m, 1H), 5.00- 4.85 (m, 1H), 4.70-4.59 (m, 1H), 4.18-4.05 (m, 1H) | 425, 427 |
| 87 | 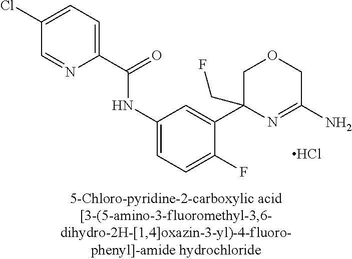 [SMILES: NC1=NC(CF)(c2cc(NC(=O)c3ccc(Cl)cn3)ccc2F)COC1 ; Cl] | 10.95-10.90 (m, 2H), 9.60-9.55 (m, 1H), 8.81- 8.78 (m, 2H), 8.23-8.21 (m, 1H), 8.18 (d, 1H), 8.08-8.05 (m, 1H), 8.04- 8.02 (m, 1H), 7.37-7.32 (m, 1H), 5.00-4.70 (m, 2H), 4.69-4.59 (m, 2H), 4.19-4.09 (m, 2H) | 381, 383 |
| 88 | 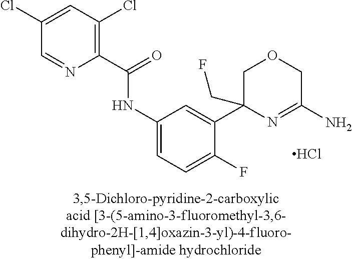 [SMILES: NC1=NC(CF)(c2cc(NC(=O)c3ncc(Cl)cc3Cl)ccc2F)COC1 ; Cl] | 11.02-10.95 (m, 2H), 9.60 (s, 1H), 8.85 (s, 1H), 8.74 (s, 1H), 8.47 (s, 1H), 7.94-7.87 (m, 1H), 7.85-7.79 (m, 1H), 7.40-7.32 (m, 1H), 5.02- 4.83 (m, 2H), 4.69-4.59 (m, 2H), 4.19-4.07 (m, 2H) | 415, 417 |
| 89 | 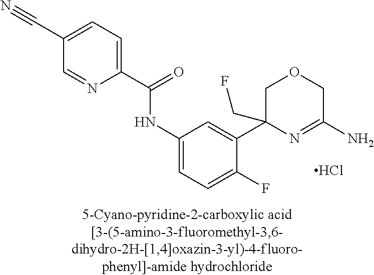 [SMILES: N#Cc1ccc(C(=O)Nc2ccc(F)c(C3(CF)COCC(N)=N3)c2)nc1 ; Cl] | 11.07 (s, 1H), 10.88 (s, 1H), 9.53 (s, 1H), 9.21 (s, 1H), 8.74 (s, 1H), 8.60 (dd, 1H), 8.30 (d, 1H), 8.10-8.03 (m, 2H), 7.40-7.32 (m, 1H), 5.03- 4.83 (m, 2H), 4.69-4.58 (m, 2H), 4.17-4.08 (m, 2H) | 372 |
| 90 | 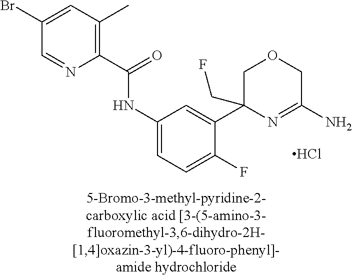 [SMILES: Cc1cc(Br)cnc1C(=O)Nc1ccc(F)c(C2(CF)COCC(N)=N2)c1 ; Cl] | 10.89 (s, 1H), 10.78 (s, 1H), 9.54 (s, 1H), 8.77 (s, 1H), 8.67 (s, 1H), 8.18 (s, 1H), 8.00-7.96 (m, 1 H), 7.90 (dd, 1H), 7.37-7.29 (m, 1H), 5.01- 4.82 (m, 2H), 4.69-4.58 (m, 2H), 4.17-4.08 (m, 2H), 2.55 (s, 3H) | 439, 441 |
| 91 | 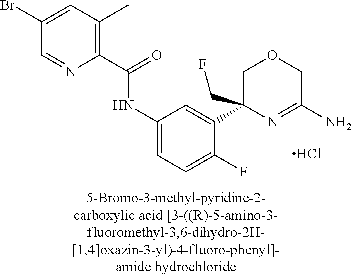 [SMILES: Cc1cc(Br)cnc1C(=O)Nc1ccc(F)c([C@]2(CF)COCC(N)=N2)c1 ; Cl] | 10.84 (s, 1H), 10.78 (s, 1H), 9.54-9.46 (m, 1H), 8.73-8.66 (m, 2H), 8.22- 8.19 (m, 1H), 8.03-7.96 (m, 1H), 7.96-7.90 (m, 1H), 7.40-7.30 (m, 1H), 5.08-4.95 (m, 1H), 4.95- 4.82 (m, 1H), 4.72-4.59 (m, 2H), 4.20-4.10 (m, 2H), 2.58 (s, 3H) | 439, 441 |
| 92 | 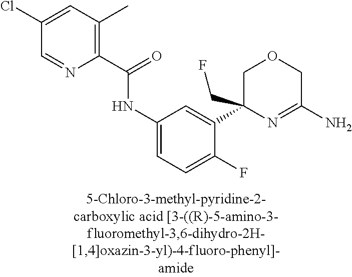 [SMILES: Cc1cc(Cl)cnc1C(=O)Nc1ccc(F)c([C@]2(CF)COCC(N)=N2)c1] | 10.51 (s, 1H), 8.55 (d, 1H), 8.00 (d, 1H), 7.91 (dd, 1H), 7.79-7.73 (m, 1H), 7.15-7.08 (m, 1H), 5.90 (s, 2H), 4.55-4.48 (m, 1H), 4.43-4.36 (m, 1H), 4.00-3.77 (m, 4H), 2.53 (s, 3H) | 395, 397 |
| 93 | 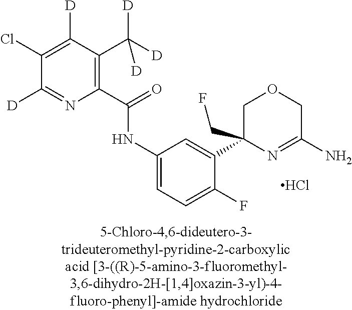 [SMILES: [2H]c1nc(C(=O)Nc2ccc(F)c([C@]3(CF)COCC(N)=N3)c2)c(C([2H])([2H])[2H])c([2H])c1Cl ; Cl] | 10.85 (d, 1H), 10.78 (s, 1H), 9.50 (d, 1H), 8.71 (d, 1H), 8.01-7.95 (m, 1H), 7.95-7.88 (m, 1H), 7.37-7.30 (m, 1H), 5.03- 4.89 (m, 2H), 4.69-4.58 (m, 2H), 4.18-4.08 (m, 2H) | 400, 402 |
| 94 | 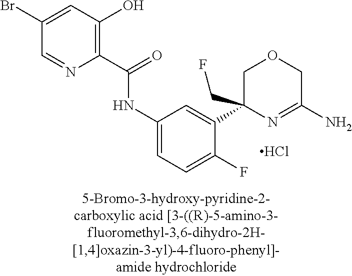 [SMILES: NC1=N[C@](CF)(c2cc(NC(=O)c3ncc(Br)cc3O)ccc2F)COC1 ; Cl] | 12.25-12.12 (m, 1H), 11.14 (s, 1H), 10.92- 10.83 (m, 1H), 9.58-9.47 (m, 1H), 8.79-8.68 (m, 1H), 8.39 (d, 1H), 8.04- 7.93 (m, 2H), 7.91 (d, 1H), 7.45-7.34 (m, 1H), 5.08-4.95 (m, 1H), 4.95- 4.81 (m, 1H), 4.72-4.58 (m, 2H), 4.21-4.09 (m, 2H) | 441, 443 |
| 95 | 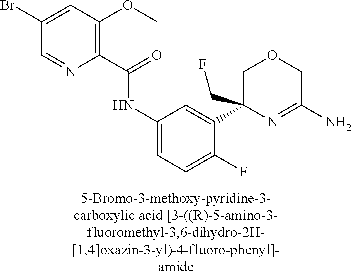 [SMILES: COc1cc(Br)cnc1C(=O)Nc1ccc(F)c([C@]2(CF)COCC(N)=N2)c1] | 10.48 (s, 1H), 8.35 (d, 1H), 7.96 (d, 1H), 7.91- 7.85 (m, 1H), 7.79-7.71 (m, 1H), 7.19-7.08 (m, 1H), 5.92 (s, 2H), 4.59- 4.52 (m, 1H), 4.46-4.39 (m, 1H), 4.05-3.80 (m, 7H) | 455, 457 |

### Example 96

### 5-Bromo-pyridine-2-carboxylic acid[3-((S)-3-amino-5-difluoromethyl-2,5,6,7-tetrahydro-[1,4]oxazepin-5-yl)-4-fluoro-phenyl]-amide hydrochloride

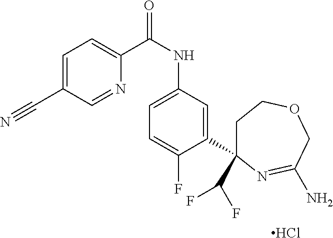 [SMILES: N#Cc1ccc(C(=O)Nc2ccc(F)c([C@]3(C(F)F)CCOCC(N)=N3)c2)nc1 ; Cl]

### a) 1-(5-Bromo-2-fluoro-phenyl)-2,2-difluoro-ethanone

A solution of diisopropyl amine (17.77 ml, 126 mmol) in 320 ml THF was cooled to −75° C. and brought under N₂ atmosphere. A 1.6 M solution of BuLi in hexane (79 ml, 126 mmol) was added. When the LDA solution had cooled down again, 1-fluoro-4-bromobenzene was added. The reaction temperature was kept below −60° C. After 2.5 h ethyl difluoro acetate (15.60 g, 126 mmol) was added rapidly and after 15 minutes, the reaction mixture was warmed to −40° C. After 15 minutes the mixture was quenched by pouring it on ice-cold 1N HCl. The mixture was extracted with petroleum ether (B.p. 40-60° C.) and the extract was dried with MgSO₄.H₂O. Chromatography on silica gel with hexane/TBME 9/1->6/1 gave 22.1 g yellow liquid.

Rf (hexanes/EtOAc 6/1)=0.28

¹H-NMR (CDCl₃, 360 MHz): 8.09 (dd, 1H), 7.79 (ddd, 1H), 7.17 (t, 1H), 6.44 (t, J=45 Hz, 1H).

### b) [1-(5-Bromo-2-fluoro-phenyl)-2,2-difluoro-eth-(Z)-ylidene]-carbamic acid tert-butyl ester

A suspension of 1-(5-bromo-2-fluoro-phenyl)-2,2-difluoro-ethanone (16.0 g, 63.2 mmol) and N-(triphenylphosphoranylidene)-carbamic acid 1,1-dimethylethyl ester (CAS 68014-21-1) (26.3 g, 69.6 mmol) in 12 ml toluene was stirred at 100° C. for 2 days. The suspension became clear. After being cooled down somewhat, hexane was added till crystallization of triphenylphosphine oxide started. The mixture was filtered and the filtrate was purified by chromatography on silica gel with hexane/TBME 1-5% to give 11.37 g of the title compound as a yellow liquid.

Rf (hexane/EtOAc 6/1)=0.65

¹H-NMR (DMSO-d6, 360 MHz): 7.88 (dd, 1H), 7.71 (br, 1H), 7.47 (t, 1H), 6.88 (br t, J=54 Hz, 1H), 1.29 (br s, 9H).

### c) [1-(5-Bromo-2-fluoro-phenyl)-1-difluoromethyl-but-3-enyl]-carbamic acid tert-butyl ester

To a solution of [1-(5-bromo-2-fluoro-phenyl)-2,2-difluoro-eth-(Z)-ylidene]-carbamic acid tert-butyl ester (9.61 g, 27.3 mmol) in 114 ml THF at −75° was added dropwise allylmagnesium chloride solution 2 mol/L in THF (15.0 ml, 30 mmol). The reaction temperature was not allowed to exceed −60° C. After 10 minutes the reaction was quenched with 10% aqueous NH₄Cl and extracted with TBME. The organic phase was washed with brine, dried with Na₂SO₄ and evaporated. The crude product was chromatographed on silica gel with 1-5% TBME/hexane to give 10.39 g of the title compound.

HPLC: Rt_{H3}=3.449 min; ESIMS [M+Na]⁺=416, 418 (1Br);

¹H-NMR (CDCl₃, 360 MHz): 7.45 (dd, 1H), 7.35 (ddd, 1H), 6.88 (dd, 1H), 6.28 (t, J=54 Hz), 1H), 5.72-5.60 (m, 1H), 5.13 (d, 1H), 5.12 (d, 1H), 5.00 (br s, 1H), 3.00-2.80 (m, 2H), 1.32 (br s, 9H).

### d) [1-(5-Bromo-2-fluoro-phenyl)-1-difluoromethyl-3-hydroxy-propyl]-carbamic acid tert-butyl ester

A suspension of [1-(5-bromo-2-fluoro-phenyl)-1-difluoromethyl-but-3-enyl]-carbamic acid tert-butyl ester (5.11 g, 12.96 mmol) and NaHCO₃ (1.63 g, 19.44 mmol) in 90 ml DCM and 30 ml MeOH was cooled to −75° C. A mixture of O₃ in oxygen gas was introduced till the blue color persisted. The excess ozone was removed by bubbling through oxygen gas for 10 minutes. NaBH₄ (0.981 g, 25.9 mmol) was added as a solid in three portions. The mixture was stirred 10 min at −75° C. and then allowed to warm to 0° C. After 30 min the mixture was poured onto ice-cold 1N HCl and extracted with TBME. The organic phase was washed with 1N HCl, brine, dried with MgSO₄.H₂O and evaporated. Chromatography on silica gel (hexanes/15-35% EtOAc) provided 4.75 g of the title compound as a colorless resin.

HPLC: Rt_{H6}=2.359 min; ESIMS [M+Na]⁺=420, 422 (1Br);

¹H-NMR (DMSO-d6, 360 MHz): 7.68 (br, 1H), 7.60-7.54 (m, 1H), 7.47 (dd, 1H), 7.20 (dd, 1H), 6.57 (t, J=54 Hz, 1H), 4.77 (t, 1H), 3.52-3.34 (m, 2H), 2.29 (br s, 2H), 1.36 (br, s, 9H).

### e) N-[1-(5-Bromo-2-fluoro-phenyl)-1-difluoromethyl-3-hydroxy-propyl]-2-chloro-acetamide

[1-(5-Bromo-2-fluoro-phenyl)-1-difluoromethyl-3-hydroxy-propyl]-carbamic acid tert-butyl ester (4.75 g, 11.93 mmol) was dissolved in 89 ml 4N HCl in dioxane. The mixture was stirred 1 h and evaporated to give 4.2 g of a white solid. The solid was suspended in 60 ml ACN and K₂CO₃ (6.59 g, 7.7 mmol) was added. The stirred suspension was cooled to 0° C. and chloroacetyl chloride (4.04 g, 35.8 mmol) was added dropwise. The mixture was stirred at 25° C. overnight. The mixture was diluted with TBME, washed with water and brine, dried with MgSO₄.H₂O and evaporated to give 5.25 g of the crude diacylated product. This crude intermediate was dissolved in 60 mL of MeOH and K₂CO₃ (330 mg, 2.39 mmol) was added. After 30 minutes, the reaction mixture was partitioned between water and TBME. The layers were separated and washed with brine and TBME. The combined organic layers were dried over MgSO₄.H₂O and evaporated. The crude product was purified on a silica gel column by eluting with hexane/EtOAc 4/1->3/1->2/1. Pure fractions were combined and evaporated to give 3.81 g of the title compound as a colorless resin.

HPLC: Rt_{H3}=3.097 min; ESIMS [M+Na]⁺=374, 376 (1Br);

¹H-NMR (CDCl₃, 360 MHz): 8.56 (br, s, 1H), 7.53 (dd, 1H), 7.49-7.43 (m, 1H), 6.99 (dd, 1H), 6.79 (t, J=54 Hz, 1H), 4.14-4.02 (m, 3H), 3.88-3.79 (m, 1H), 2.45 (t, 2H), 1.19 (d, 1H).

### f) 5-(5-Bromo-2-fluoro-phenyl)-5-difluoromethyl-[1,4]oxazepan-3-one

To a refluxing solution of potassium tert-butylate (1.63 g, 14.52 mmol) in 555 ml t-BuOH was added dropwise a solution of) N-[1-(5-bromo-2-fluoro-phenyl)-1-difluoromethyl-3-hydroxy-propyl]-2-chloro-acetamide (2.72 g, 7.26 mmol) in 45 ml THF over a period of 40 minutes. The reaction mixture was cooled down and quenched with 1N HCl. EtOAc was added and the organic layer was washed with brine, dried with Na₂SO₄ and evaporated. The crude product was crystallized from DCM/TBME to provide the title compound as white crystals.

HPLC: Rt_{H3}=2.989 min; ESIMS [M+H]⁺=338, 340 (1Br);

¹H-NMR (DMSO-d6, 360 MHz): 8.38 (s, 1H), 7.70-7.63 (m, 2H), 7.29 (dd, 1H), 6.20 (t, J=54 Hz, 1H), 4.17 (d, 1H), 4.04 (d, 1H), 3.80-3.73 (m, 1H), 3.45-3.34 (m, 1H), 2.73-2.56 (m, 2H).

### g) [5-(5-Amino-2-fluoro-phenyl)-5-difluoromethyl-2,5,6,7-tetrahydro-[1,4]oxazepin-3-yl]-carbamic acid tert-butyl ester

This compound was obtained from 5-(5-bromo-2-fluoro-phenyl)-5-difluoromethyl-[1,4]oxazepan-3-one by a similar sequence as described for example n75 step c) and example 42 steps g) to j) as a colorless foam.

HPLC: Rt_{H3}=2.410 min; ESIMS [MH+H₂O]⁺=392,
Rt_{H3}=2.595 min; ESIMS [MH]⁺=374;

¹H-NMR (CDCl₃, 360 MHz, broad signals due to rotamers): 11.09 (s, 1H), 6.96-6.88 (dd, 1H), 6.71-6.62 (m, 2H), 6.11 (t, J=54 Hz, 1H), 4.47-4.25 (m, 2H), 3.89-3.80 (m, 1H), 3.74-3.55 (m, 3H), 2.79-2.69 (m, 1H), 2.65-2.50 (m, 1H), 1.58 (s, 9H).

### h) [(S)-5-(5-Amino-2-fluoro-phenyl)-5-difluoromethyl-2,5,6,7-tetrahydro-[1,4]oxazepin-3-yl]-carbamic acid tert-butyl ester

Racemic [5-(5-amino-2-fluoro-phenyl)-5-difluoromethyl-2,5,6,7-tetrahydro-[1,4]oxazepin-3-yl]-carbamic acid tert-butyl ester (1.62 g, 4.04 mmol) was separated via a VWR prep HPLC System on a Chiralpak AD 20 um 4×50×100 (4×SMB columns) column; eluent: heptane/ethanol 70/30; flow=65 ml/min; UV detection at 220 nm. As a result, 754 mg of the desired title compound ((S)-isomer) was obtained as the first eluting isomer. Purity: >99.5% ee.

¹H-NMR (CDCl₃, 360 MHz, broad signals due to rotamers): 11.08 (s, 1H), 6.96-6.88 (dd, 1H), 6.71-6.63 (m, 2H), 6.11 (t, J=54 Hz, 1H), 4.47-4.26 (m, 2H), 3.89-3.81 (m, 1H), 3.80-3.56 (m, 3H), 2.78-2.70 (m, 1H), 2.64-2.51 (m, 1H), 1.58 (s, 9H). α_{D}=−166.5° (c=1, solvent=CHCl₃)

### i) ((S)-5-{5-[(5-Cyano-pyridine-2-carbonyl)-amino]-2-fluoro-phenyl}-5-difluoromethyl-2,5,6,7-tetrahydro-[1,4]oxazepin-3-yl)-carbamic acid tert-butyl ester

A solution of [(S)-5-(5-amino-2-fluoro-phenyl)-5-difluoromethyl-2,5,6,7-tetrahydro-[1,4]oxazepin-3-yl]-carbamic acid tert-butyl ester (51.2 mg, 0.137 mmol), 5-cyano-2-pyridinecarboxylic acid (30.5 mg, 0.206 mmol) and HOAT (33.6 mg, 0.247 mmol) in 0.6 mL DMF was cooled to 0-5° C. EDC (39.4 mg, 0.206 mmol) and DIPEA (35.4 mg, 0.274 mmol) were added. The resulting solution was allowed to warm up to rt over night. The reaction mixture was then partitioned between saturated aqueous NaHCO₃ solution and EtOAc. The layers were separated, washed with saturated aqueous NaHCO₃ solution, brine and EtOAc. The combined organic layers were dried over MgSO₄.H₂O and evaporated. The crude product was purified on a silica gel column by eluting with hexane/EtOAc 3/1->1.5/1 to give 67.7 mg of the title compound as a colorless resin.

HPLC: Rt_{H1}=2.492 min; ESIMS=[M+H]+504;

¹H-NMR (CDCl₃, 360 MHz, broad signals due to rotamers): 11.10 (s, 1H), 9.85 (s, 1H), 8.86 (s, 1H), 8.35 (d, 1H), 8.28-8.19 (m, 1H), 8.14 (dd, 1H), 7.32-7.25 (m, 1H), 7.10 (t, 1H), 6.08 (t, J=54 Hz, 1H), 4.43-4.15 (m, 2H), 3.80-3.71 (m, 1H), 3.59-3.42 (m, 1H), 2.74-2.65 (m, 1H), 2.62-2.47 (m, 1H), 1.49 (s, 9H).

### j) 5-Bromo-pyridine-2-carboxylic acid[3-((S)-3-amino-5-difluoromethyl-2,5,6,7-tetrahydro-[1,4]oxazepin-5-yl)-4-fluoro-phenyl]-amide hydrochloride

((S)-5-{5-[(5-Cyano-pyridine-2-carbonyl)-amino]-2-fluoro-phenyl}-5-difluoromethyl-2,5,6,7-tetrahydro-[1,4]oxazepin-3-yl)-carbamic acid tert-butyl ester (67.7 mg, 0.134 mmol) was dissolved in 0.75 mL dichloromethane and 0.25 mL trifluoroacetic acid. The solution was stirred at for 45 minutes and then evaporated at room temperature. The residue was dissolved in EtOAc and extracted with saturated aqueous NaHCO₃ solution. The layers were washed with brine and EtOAc. The combined organic layers were dried over Na₂SO₄ and evaporated. The crude product was dissolved in THF, 0.2 mL HCl solution 1 mol/L in diethyl ether was added and the mixture was evaporated. The residue was crystallized from ethanol and TBME to give 48 mg of the title compound as white crystals.

HPLC: Rt_{H3}=2.629 min; ESIMS [M+H]⁺=404.0;

¹H-NMR (DMSO, 600 MHz): 11.11 (s, 1H), 10.20 (s, 1H) 9.75 (s, 1H), 9.22 (s, 1H), 8.78 (s, 1H), 8.60 (d, 1H), 8.30 (d, 1H), 8.12-8.07 (m, 1H), 8.01-7.97 (m, 1H), 7.35 (dd, 1H), 6.55 (t, J=54 Hz, 1H), 4.77 (d, 1H), 4.52 (d, 1H), 3.89-3.83 (m, 1H), 3.50-3.39 (m, 1H), 2.79-2.68 (m, 2H).

### Example 97

The compound listed in Table 13 can be prepared by a procedure analogous to that used in example 96, using 4N HCl in dioxane in step j).

TABLE 13

| | | | MS |
|---|---|---|---|
| | | ¹H-NMR | [m/z; |
| Example | Compound | (δ; DMSO-d₆) | (M + 1)⁺] |
| 97 | 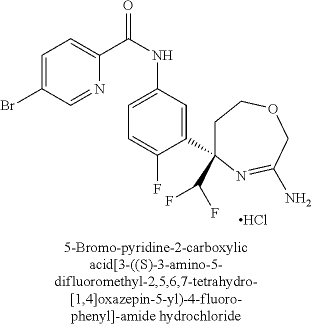 [SMILES: NC1=N[C@@](c2cc(NC(=O)c3ccc(Br)cn3)ccc2F)(C(F)F)CCOC1 ; Cl] | 10.97 (s, 1H), 10.17 (s, 1H) 9.74 (s, 1H), 8.87 (s, 1H), 8.75 (s, 1H), 8.34 (d, 1H), 8.11-8.05 (m, 2H), 7.99- 7.96 (m, 1H), 7.35 (dd, 1H), 6.54 (t, J = 54 Hz, 1H), 4.77 (d, 1H), 4.51 (d, 1H), 3.89-3.84 (m, 1H), 3.48-3.41 (m, 1H), 2.79- 2.68 (m, 2H) | 457, 459 |

### Example 98

### 5-Cyano-pyridine-2-carboxylic acid [3-(3-amino-5-trifluoromethyl-2,5,6,7-tetrahydro-[1,4]oxazepin-5-yl)-4-fluoro-phenyl]-amide hydrochloride

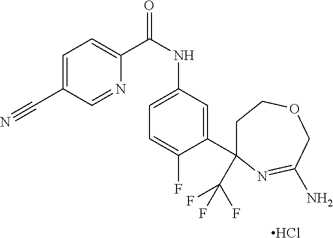 [SMILES: N#Cc1ccc(C(=O)Nc2ccc(F)c(C3(C(F)(F)F)CCOCC(N)=N3)c2)nc1 ; Cl]

### a) [2,2,2-Trifluoro-1-(2-fluoro-phenyl)-eth-(Z)-ylidene]-carbamic acid tert-butyl ester

A suspension of 1-(2-fluoro-phenyl)-2,2,2-trifluoro-ethanone (CAS 124004-75-7) (17.0 g, 88 mmol) and N-(triphenylphosphoranylidene)-carbamic acid 1,1-dimethylethyl ester (CAS 68014-21-1) (36.7 g, 97 mmol) in 17 ml toluene was stirred at 120° C. for 18 h. The suspension became clear. After being cooled down hexane was added till crystallization of triphenylphosphine oxide started. The mixture was filtered and the filtrate was purified by chromatography on silica gel with 1-5% TBME/hexanes to yield 11.37 g of a yellow liquid.

Rf (Hex/TBME 95/5)=0.18

HPLC: Rt_{H6}=3.168 min; ESIMS [M+Na]⁺=314,

¹H-NMR (CDCl3, 360 MHz): 7.59-7.52 (m, 1H), 7.42-7.35 (m, 1H), 7.30-7.18 (m, 2H), 1.31 (s, 9H).

### b) [1-(2-Fluoro-phenyl)-1-trifluoromethyl-but-3-enyl]-carbamic acid tert-butyl ester

To a solution of [2,2,2-Trifluoro-1-(2-fluoro-phenyl)-eth-(Z)-ylidene]-carbamic acid tert-butyl ester (16.63 g, 57.1 mmol) in 170 mL THF at −75° was added dropwise allylmagnesium chloride solution 2 mol/L in THF (31.4 ml, 62.8 mmol). The reaction temperature was not allowed to exceed −60° C. After 30 minutes, the reaction was quenched with 10% aqueous NH₄Cl and extracted with TBME. The organic phase was washed with brine, dried with MgSO4.H₂O and evaporated. The crude product was chromatographed on silica gel with hexane/TBME 95/5 to give 18.52 g of the title compound.

HPLC: Rt_{H3}=3.296 min; ESIMS [M+Na]⁺=356;

¹H-NMR (CDCl₃, 360 MHz): 7.49 (t, 1H), 7.41-7.33 (m, 1H), 7.19 (t, 1H), 7.14-7.06 (dd, 1H), 5.92-5.78 (m, 2H), 5.30-5.19 (m, 2H), 3.37-3.18 (br, m, 2H), 1.40 (br, s, 9H).

### c) [1-(2-Fluoro-phenyl)-3-hydroxy-1-trifluoromethyl-propyl]-carbamic acid tert-butyl ester

A suspension of [[1-(2-fluoro-phenyl)-1-trifluoromethyl-but-3-enyl]-carbamic acid tert-butyl ester (9.27 g, 27.8 mmol) and NaHCO₃ (3.50 g, 41.7 mmol) in 168 ml DCM and 56 ml MeOH was cooled to −75° C. A mixture of O₃ in oxygen gas was introduced till the blue color persisted. The excess ozone was removed by bubbling through oxygen gas for 10 minutes. Solid NaBH₄ (2.10 g, 55.6 mmol) was added in two portions. The mixture was stirred 10 min at −75° C. and then allowed to warm to 0° C. After 30 minutes, the mixture was poured onto ice-cold 1N HCl and extracted with TBME. The organic phase is washed with 1N HCl, brine, dried with MgSO₄.H₂O and evaporated. Crystallization from hexane provided 7.83 g of the title compound as white crystals.

HPLC: Rt_{H1}=2.738 min; ESIMS [M+Na]⁺=360;

¹H-NMR (DMSO-d6, 360 MHz): 7.82 (br, s, 1H), 7.46-7.35 (m, 2H), 7.27-7.17 (m, 2H), 4.79 (t, 1H), 3.51-3.36 (m, 2H), 2.48-2.31 (m, 2H), 1.32 (br, s, 9H).

### d) 2-Chloro-N-[1-(2-fluoro-phenyl)-3-hydroxy-1-trifluoromethyl-propyl]-acetamide

[1-(2-Fluoro-phenyl)-3-hydroxy-1-trifluoromethyl-propyl]-carbamic acid tert-butyl ester (7.83 g, 23.21 mmol) was dissolved in 116 ml 4N HCl in dioxane. The mixture was stirred 1 h and evaporated to give 6.42 g of a white solid. The solid was dissolved in 65 ml dichloromethane and pyridine (11.3 mL, 139 mmol). The solution was cooled to −15° C. and chloroacetyl chloride (5.50 g, 48.7 mmol) was added dropwise. The temperature was kept below −5° C. Afterwards, the mixture was allowed to warm up to room temperature. After 40 minutes, the reaction mixture was partitioned between 1N HCl and TBME. The layers were separated, washed with brine and TBME. The combined organic layers were dried over MgSO₄.H₂O and evaporated. The crude product was purified on silica gel by eluting with hexane/EtOAc 3/1->2/1 to give 5.46 g of a mixture of diacylated and O-acylated product. This mixture was dissolved in 80 mL dichloromethane. DIPEA (15.8 mL, 90.70 mmol) was added and the reaction mixture was cooled to −75° C. and chloroacetyl chloride (9.89 g, 87.57 mmol) was added dropwise. Afterwards, the mixture was stirred without cooling bath for 15′. The reaction mixture was partitioned between 1N HCl and TBME. The layers were separated, washed with brine and TBME. The combined organic layers were dried over MgSO₄.H₂O and evaporated. The crude product was purified on a silica gel column by eluting with hexane/EtOAc 3/1 to give 3.71 g of the diacylated compound.

In order to get the title compound, the diacylated compound was dissolved in 50 mL of MeOH and K₂CO₃ (657 mg, 4.76 mmol) was added. After 45 minutes, the reaction mixture was partitioned between water and TBME. The layers were separated, washed with brine and TBME. The combined organic layers were dried over MgSO₄.H₂O and evaporated. The crude product was purified on a silca gel column by eluting with hexane/EtOAc 3/1->2/1->1.5/1 to give 1.77 g of the title compound as a yellow resin.

HPLC: Rt_{H3}=2.889 min; ESIMS [M+H]⁺=314;

¹H-NMR (DMSO-d6, 360 MHz): 9.10 (br, s, 1H), 7.48-7.39 (m, 2H), 7.27-7.17 (m, 2H), 4.77 (br, t, 1H), 4.25 (dd, 2H), 3.54-3.40 (m, 2H), 2.72-2.61 (m, 1H), 2.58-2.48 (m, 1H)

### e) 5-(2-Fluoro-phenyl)-5-trifluoromethyl-[1,4]oxazepan-3-one

To a refluxing solution of potassium tert-butylate (1.31 g, 11.29 mmol) in 43 ml t-BuOH was added dropwise a solution of 2-chloro-N-[1-(2-fluoro-phenyl)-3-hydroxy-1-trifluoromethyl-propyl]-acetamide (1.77 g, 5.64 mmol) in 35 ml THF over a period of 60 minutes. The reaction mixture was cooled down and quenched with 1N HCl. EtOAc was added and the organic layer was washed with brine, dried with MgSO₄.H₂O and evaporated. The crude product was purified on a silica gel column by eluting with hexane/EtOAc 3/1->2.5/1 to give 1.19 g of the title compound as white crystals.

HPLC: Rt_{H3}=2.943 min; ESIMS [M+H]⁺=278;

¹H-NMR (CDCl3, 360 MHz): 7.58 (t, 1H), 7.51-7.44 (m, 1H), 7.33-7.27 (m, 1H), 7.18 (dd, 1H), 6.47 (br, s, 1H), 4.16 (dd, 2H), 4.00-3.91 (m, 1H), 3.82-3.72 (m, 1H), 3.19-3.11 (m, 1H), 2.81-2.68 (m, 1H).

### f) 5-(2-Fluoro-phenyl)-5-trifluoromethyl-[1,4]oxazepane-3-thione

To a solution of 5-(2-fluoro-phenyl)-5-trifluoromethyl-[1,4]oxazepan-3-one (1.19 g, 4.29 mmol) in 15 mL THF was added Lawesson's reagent (955 mg, 2.36 mmol). The reaction mixture was stirred at room temperature over night. The mixture was then partitioned between aqueous Na₂CO₃ solution (2 mol/L) and TBME. The layers were separated, washed with aqueous Na₂CO₃ solution (2 mol/L), brine and TBME. The combined organic layers were dried over MgSO₄.H₂O and evaporated. The crude product was purified on a silica gel column by eluting with hexane/EtOAc 95/5->90/10 to give 1.25 g of the title compound as a yellow resin.

HPLC: Rt_{H3}=2.620 min; ESIMS [M+H]⁺=294;

¹H-NMR (CDCl3, 360 MHz): 8.42 (br, s, 1H), 7.54-7.45 (m, 2H), 7.35-7.27 (m, 1H), 7.20 (dd, 1H), 4.54 (dd, 2H), 4.05-3.97 (m, 1H), 3.84-3.74 (m, 1H), 3.17-3.08 (m, 1H), 2.85-2.73 (m, 1H).

### g) 5-(2-Fluoro-phenyl)-5-trifluoromethyl-2,5,6,7-tetrahydro-[1,4]oxazepin-3-ylamine

5-(2-Fluoro-phenyl)-5-trifluoromethyl-[1,4]oxazepane-3-thione (1.25 g, 4.26 mmol) was dissolved in NH₃ solution 7 mol/L in methanol (27 mL, 128 mmol). The sealed reaction vessel was stirred over night at rt. The reaction mixture was evaporated, dissolved in TBME and extracted with 1N HCl. The layers were separated, washed with water and TBME. The aqueous layers were combined, basified by addition of solid K₂CO₃ and extracted with dichloromethane four times. The combined CH₂Cl₂ layers were dried over MgSO₄.H₂O and evaporated to give 1.12 g of the title compound as white crystals.

HPLC: Rt_{H3}=2.475 min; ESIMS [M+H]⁺=277;

¹H-NMR (CDCl3, 360 MHz): 7.50 (t, 1H), 7.32-7.23 (m, 1H), 7.09 (t, 1H), 7.04-6.96 (m, 1H), 4.62 (br, s, 2H), 3.95 (m, 2H), 3.76 (d, 1H), 3.73-3.63 (m, 1H), 2.92-2.84 (m, 1H), 2.46-2.34 (m, 1H).

### h) 5-(2-Fluoro-5-nitro-phenyl)-5-trifluoromethyl-2,5,6,7-tetrahydro-[1,4]oxazepin-3-ylamine

To a solution of 5-(2-fluoro-phenyl)-5-trifluoromethyl-2,5,6,7-tetrahydro-[1,4]oxazepin-3-ylamine (1.12 g, 4.05 mmol) in 12 mL concentrated sulfuric acid (95%) was added potassium nitrate (533 mg, 5.27 mmol) in two portions. The reaction mixture was stirred at rt for 30 minutes, it was then poured onto ice water and TBME was added. The layers were separated, washed with water and TBME. The combined aqueous layers were basified with solid Na₂CO₃ and extracted with EtOAc. The EtOAc layers were dried over MgSO₄.H₂O and evaporated to give 1.29 g of the title compound was a white solid.

HPLC: Rt_{H3}=2.433 min; ESIMS [M+H]⁺=322;

¹H-NMR (DMSO-d6, 360 MHz): 8.47 (dd, 1H), 8.39-8.31 (m, 1H), 7.58 (dd, 1H), 6.48 (br, s, 2H), 4.23 (d, 1H), 3.96 (d, 1H), 3.93-3.85 (m, 1H), 3.55-3.45 (m, 1H), 2.85-2.76 (m, 1H), 2.61-2.53 (m, 1H).

### i) [5-(2-Fluoro-5-nitro-phenyl)-5-trifluoromethyl-2,5,6,7-tetrahydro-[1,4]oxazepin-3-yl]-carbamic acid tert-butyl ester

To a suspension of 5-(2-fluoro-5-nitro-phenyl)-5-trifluoromethyl-2,5,6,7-tetrahydro-[1,4]oxazepin-3-ylamine (1.29 g, 4.02 mmol) in 10 mL dichlormethane and 15 mL THF were added DIPEA (779 mg, 6.02 mmol) and di-tert-butyldicarbonate (1.14 g, 5.22 mmol). The reaction mixture was stirred over night and then heated to 40° C. for 24 h. Additional DIPEA (104 mg, 0.8 mmol) and di-tert-butyldicarbonate (175 mg, 0.8 mmol) were added. The mixture was stirred at 40° C. for another 8 h. The reaction mixture was then evaporated and purified on a silica gel column by eluting with hexane/TBME 9/1->7/1 to give 1.69 g of the title compound as a white foam.

HPLC: Rt_{H1}=3.445 min; ESIMS=[M-tBu]⁺366;

¹H-NMR (CDCl₃, 360 MHz): 8.41-8.34 (m, 1H), 8.30-8.24 (m, 1H), 7.39 (br, s, 1H), 7.28 (t, 1H), 5.12 (d, 1H), 4.52 (d, 1H), 3.90-3.78, (m, 2H), 3.05-2.97 (m, 1H), 2.71-2.59 (m, 1H), 1.53 (s, 9H).

### j) [5-(5-Amino-2-fluoro-phenyl)-5-trifluoromethyl-2,5,6,7-tetrahydro-[1,4]oxazepin-3-yl]-carbamic acid tert-butyl ester

A solution of [5-(2-fluoro-5-nitro-phenyl)-5-trifluoromethyl-2,5,6,7-tetrahydro-[1,4]oxazepin-3-yl]-carbamic acid tert-butyl ester (1.69 g, 4.01 mmol) in 10 mL ethanol and 10 mL THF was brought under nitrogen atmosphere and 500 mg of 5% Pd on charcoal was added. The reaction mixture was then stirred under a hydrogen atmosphere (balloon) for 6 hours. Then it was filtered over celite and evaporated. The crude product was crystallized from hexane/TBME to give 1.38 g of the title compound as white crystals.

HPLC: Rt_{H3}=3.006 min; ESIMS=[M+H]⁺392;

¹H-NMR (CDCl₃, 360 MHz, broad signals due to rotamers): 7.01-6.86 (m, 1H), 6.76-6.62 (m, 2H), 4.96 (d, 1H), 4.58 (d, 1H), 4.31-4.18 (dd, 1H), 4.03-3.73 (m, 2H), 3.69 (s, 1H), 3.57 (s, 1H), 3.13-2.90 (dd, 1H), 2.70-2.46 (m, 1H), 1.58 (s, 9H).

### k) (5-{5-[(5-Cyano-pyridine-2-carbonyl)-amino]-2-fluoro-phenyl}-5-trifluoromethyl-2,5,6,7-tetrahydro-[1,4]oxazepin-3-yl)-carbamic acid tert-butyl ester

A solution of [5-(5-amino-2-fluoro-phenyl)-5-trifluoromethyl-2,5,6,7-tetrahydro-[1,4]oxazepin-3-yl]-carbamic acid tert-butyl ester (50 mg, 0.128 mmol), 5-cyano-2-pyridinecarboxylic acid (28.4 mg, 0.192 mmol) and HOAT (31.3 mg, 0.230 mmol) in 0.5 mL DMF was cooled to 0-5° C. EDC (36.7 mg, 0.192 mmol) and DIPEA (33 mg, 0.256 mmol) were added. The resulting solution was allowed to warm up to rt over night. The reaction mixture was then partitioned between saturated aqueous NaHCO₃ solution and EtOAc. The layers were washed with saturated aqueous NaHCO₃ solution, brine and EtOAc. The combined organic layers were dried over MgSO₄.H₂O and evaporated. The crude product was purified on a silica gel column by eluting with hexane/EtOAc 4/1->3/1 to give 63.3 mg of the title compound as a white solid.

HPLC: Rt_{H1}=2.426 min; ESIMS=[M+H₂O]⁺540,
Rt_{H1}=3.177 min; ESIMS=[M+H]⁺522;

¹H-NMR (CDCl₃, 360 MHz, broad signals due to rotamers): 9.75 (s, 1H), 8.83 (s, 1H), 8.35 (d, 1H), 8.14 (dd, 1H), 8.03-7.95 (m, 1H), 7.48-7.39 (m, 1H), 7.06 (t, 1H), 4.94 (d, 1H), 4.46 (d, 1H), 4.19 (s, 1H), 3.92-3.63 (m, 2H), 3.08-2.85 (m, 1H), 2.69-2.43 (m, 1H), 1.42 (s, 9H).

### l) 5-Cyano-pyridine-2-carboxylic acid [3-(3-amino-5-trifluoromethyl-2,5,6,7-tetrahydro-[1,4]oxazepin-5-yl)-4-fluoro-phenyl]-amide hydrochloride

(5-{5-[(5-Cyano-pyridine-2-carbonyl)-amino]-2-fluoro-phenyl}-5-trifluoromethyl-2,5,6,7-tetrahydro-[1,4]oxazepin-3-yl)-carbamic acid tert-butyl ester (63.3, 0.121 mmol) was dissolved in 0.68 mL dichloromethane and 0.23 mL trifluoroacetic acid. The solution was stirred at for 45 minutes and then evaporated at room temperature. The residue was dissolved in EtOAc and extracted with saturated aqueous NaHCO₃ solution. The layers were washed with brine and EtOAc. The combined organic layers were dried over Na₂SO₄ and evaporated. The crude product was dissolved in THF, 0.3 mL HCl solution 1 mol/L in diethyl ether was added and the mixture was evaporated. The residue was crystallized from wet ethanol and TBME to give 52.4 mg of the title compound as white crystals.

HPLC: Rt_{H3}=2.814 min; ESIMS [M+H]⁺=422;

¹H-NMR (DMSO, 600 MHz): 11.15 (s, 1H), 10.73 (s, 1H) 9.95 (s, 1H), 9.22 (s, 1H), 8.95 (s, 1H), 8.60 (d, 1H), 8.29 (d, 1H), 8.12-8.06 (m, 2H), 7.40 (dd, 1H), 4.76 (d, 1H), 4.49 (d, 1H), 3.98-3.93 (m, 1H), 3.69-3.62 (m, 1H), 2.99-2.92 (s, broad, 2H).

### Example 99

The compound listed in Table 14 can be prepared by a procedure analogous to that used in example 98, using 4N HCl in dioxane in step l).

TABLE 14

| | | | MS |
|---|---|---|---|
| | | ¹H-NMR | [m/z; |
| Example | Compound | (δ; DMSO-d₆) | (M + 1)⁺] |
| 99 | 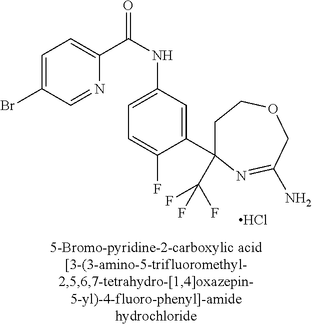 [SMILES: NC1=NC(c2cc(NC(=O)c3ccc(Br)cn3)ccc2F)(C(F)(F)F)CCOC1 ; Cl] | 11.01 (s, 1H), 10.71 (s, 1H) 9.93 (s, 1H), 8.93 (s, 1H), 8.88 (s, 1H), 8.34 (d, 1H), 8.11-8.05 (m, 3H), 7.39 (dd, 1H), 4.76 (d, 1H), 4.48 (d, 1H), 3.98-3.93 (m, 1H), 3.69-3.61 (m, 1H), 2.97-2.92 (s, broad, 2H) | 475, 477 |

### Example 100

### N-(3-(3-amino-6,6-difluoro-5-methyl-2,5,6,7-tetrahydro-1,4-oxazepin-5-yl)-4-fluorophenyl)-5-chloropicolinamide

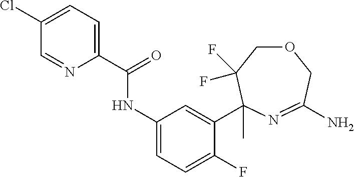 [SMILES: CC1(c2cc(NC(=O)c3ccc(Cl)cn3)ccc2F)N=C(N)COCC1(F)F]

### a) Ethyl 3-(5-bromo-2-fluorophenyl)-2,2-difluoro-3-hydroxypropanoate

To an ice cooled solution of 5-bromo-2-fluorobenzaldehyde (10.0 g, 49.2 mmol) in dry DMF (14 mL), Indium powder (8.48 g, 73.7 mmol) was added and stirred for 15 min. ethylbromodifluoroacetate (9.48 ml [14.98 g], 73.7 mmol) in dry DMF (10 mL) was added to the resultant reaction mixture and temperature of the reaction mixture was allowed to warm to rt (30° C.). Stirring continued for 24 h. TLC analysis of the reaction mixture indicated the product formation. Reaction mixture was treated with ageous saturated NH₄Cl solution and the crude product was extracted with ethyl acetate (500 mL) by washing with water, brine and the organic layer was dried over anhydrous Na₂SO₄. The organic layer was concentrated and the crude product was purified by column chromatography on silica gel using 4% Ethyl acetate in Hexane to obtain title compound as a colorless thick liquid. Yield=12.0 g (75%). TLC (5% ethyl acetate in Hexane: Rf=0.2),

¹H NMR (400 MHz, CDCl₃) δ 7.75-7.68 (m, 1H), 7.52-7.45 (m, 1H), 6.98 (t, 1H, J=7 Hz), 5.52 (dt, 1H, J=10 Hz, 4 Hz), 4.37 (q, 2H, J=5 Hz), 2.9 (d, 1H), 1.35 (t, 3H).

### b) 1-(5-bromo-2-fluorophenyl)-2,2-difluoropropane-1,3-diol

To a solution of ethyl 3-(5-bromo-2-fluorophenyl)-2,2-difluoro-3-hydroxypropanoate (20.0 g, 61.3 mmol) in MeOH (160 mL), NaBH₄ (7.0 g, 184.2 mmol) was added portion wise over a period of 30 min. at 0° C. Stirring was continued for 1 h at 0° C. and reaction was monitored by TLC. Upon complete consumption of the starting material, reaction mass was concentrated under reduced pressure and treated with saturated ammonium chloride solution. The crude reaction mass was dissolved in ethyl acetate and organic layer was washed with brine (15 mL) followed by drying over anhydrous Na₂SO₄. The organic layer was concentrated under reduced pressure to furnish title compound with sufficient purity. Yield=17 g (97%). TLC (50% ethyl acetate in Hexane): Rf=0.32),

LCMS: Rt_{H8}=0.665, [M−H]⁺=283.0, 283.9,

¹H NMR (400 MHz, CDCl₃) δ 7.76-7.70 (m, 1H), 7.51-7.42 (m, 1H), 6.90 (t, 1H, J=8.0 Hz), 5.42 (dd, 1H J=8.2 Hz, 4.0 Hz), 4.15-3.81 (m, 2H), 3.0 (s, 1H), 2.26 (s, 1H).

### c) 1-(5-bromo-2-fluorophenyl)-3-(tert-butyldimethylsilyloxy)-2,2-difluoropropan-1-ol

To an ice cooled solution of 1-(5-bromo-2-fluorophenyl)-2,2-difluoropropane-1,3-diol (17.0 g, 59.8 mmol) in dry DCM (200 mL) was added imidazole (12.2 g, 179.2 mmol) at 0° C. and stirred for 15 min. tert-butyldimethylsilylchloride (13.5 g, 89.5 mmol) was added to the resultant reaction mixture portion wise for a period of 30 min. and stirring continued for 2 h. Reaction was monitored by TLC analysis. The solids formed in the reaction mixture were separated by filtration and filtrate was concentrated under reduced pressure to obtain crude product which was purified by column chromatography on silica gel with 2% Ethylacetate in Hexane as eluent to furnish title compound as a colorless liquid. Yield=19 g (80%). TLC (20% ethyl acetate in Hexane): Rf=0.75),

LCMS: Rt_{H8}=2.09, [M+H]⁺=399.0,

¹H NMR (300 MHz, CDCl₃) δ 7.77-7.69 (m, 1H), 7.48-7.39 (m, 1H), 6.96 (t, 1H, J=9 Hz), 5.41 (dt, 1H, J=14 Hz, 3.4 Hz), 4.1-3.8 (m, 2H), 3.22 (d, 1H, J=5.2 Hz), 0.93 (s, 9.1H), 0.16 (s, 6H).

### d) 1-(5-bromo-2-fluorophenyl)-3-(tert-butyldimethylsilyloxy)-2,2-difluoropropan-1-one

A mixture of 1-(5-bromo-2-fluorophenyl)-3-(tert-butyldimethylsilyloxy)-2,2-difluoropropan-1-ol (19.0 g, mmol) and Pyridinium dichromate (90.0 g, 239.2 mmol) in Dichloromethane (200 mL) was refluxed for 16 h under constant stirring. The catalyst was filtered through a pad of celite and the filtrate was concentrated under reduced pressure to obtain brown colored thick mass. Crude product was purified by column chromatography on silica gel with 1% ethyl acetate in Hexane to obtain title compound as colorless oil. Yield=17.0 g (90%).

TLC (10% ethyl acetate in Hexane): Rf=0.56),

LCMS: Rt_{H8}=1.917, [M+H]⁺=396.7, 398.6,

¹H NMR (300 MHz, CDCl₃) δ 7.91-7.85 (m, 2H), 7.71-7.62 (m, 1H), 7.08 (t, 1H, J=8.5 Hz), 4.12 (t, 1H, J=11.5 Hz), 0.83 (s, 9H), 0.4 (6H).

### e) N-(1-(5-bromo-2-fluorophenyl)-3-(tert-butyldimethylsilyloxy)-2,2-difluoropropylidene)-2-methylpropane-2-sulfinamide

To a solution of 1-(5-bromo-2-fluorophenyl)-3-(tert-butyldimethylsilyloxy)-2,2-difluoropropan-1-one (16.0 g, 40.4 mmol) in dry THF (350 mL) was added Ti(OEt)₄ (16.7 mL, 80.4 mmol) and 2-Methyl-2-propane sulfonamide (5.8 g, 48.4 mmol) and refluxed for 16 h. Reaction mixture was concentrated under reduced pressure and the crude residue was directly purified by column chromatography on silica gel with 3% ethyl acetate in Hexane to furnish title compound as a colorless liquid. Yield=13.1 g (65.5%). TLC (10% ethyl acetate in Hexane): Rf=0.2),

LCMS Rt_{H8}=2.29 [M+H]⁺=499.9, 501.8,

¹H NMR (300 MHz, CDCl₃) δ 7.48-7.29 (m, 2H, 6.98 (m, 1H), 4.10 (t, 1H), 1.23 (d, 9H), 0.96 (d, 9H), 0.5 (d, 6H).

### f) N-(2-(5-bromo-2-fluorophenyl)-4-(tert-butyldimethylsilyloxy)-3,3-difluorobutan-2-yl)-2-methylpropane-2-sulfinamide

To a solution of N-(1-(5-bromo-2-fluorophenyl)-3-(tert-butyldimethylsilyloxy)-2,2-difluoro-propylidene)-2-methylpropane-2-sulfinamide (12 g, 24.04 mmol) in diethyl ether (120 mL) was added CH₃MgBr (3M in Diethyl ether) (41 mL, 120 mmol) at −25° C. The reaction mixture was brought to 0° C. and maintained for 30 min. Reaction mixture was again cooled to −35° C. and quenched by drop wise addition of saturated ammonium chloride solution. Organic layer was separated and washed with brine and dried over anhydrous sodium sulphate. Crude compound was purified by column chromatography on silica gel with 8% ethyl acetate in Hexane to furnish title compound as a colorless liquid. Yield=8.8 g (71%).

TLC (20% ethyl acetate in Hexane): Rf=0.33),

LCMS: Rt_{H8}=2.161 [M+H]⁺=516.1, 519.0,

¹H NMR (300 MHz, CDCl₃) δ 7.71-7.62 (m, 1H), 7.44-7.38 (m, 1H), 7.0-6.84 (m, 1H), 4.82 (d, 1H), 4.05-3.9 (m, 2H), 2.06 (s, 3H), 1.25 (s, 9H), 0.9 (s, 9H), 0.11 (s, 6H).

### g) 3-amino-3-(5-bromo-2-fluorophenyl)-2,2-difluorobutan-1-ol

To a solution of N-(2-(5-bromo-2-fluorophenyl)-4-(tert-butyldimethylsilyloxy)-3,3-difluorobutan-2-yl)-2-methylpropane-2-sulfinamide (8.8 g, 17.08 mmol) in dry MeOH (60 mL), dry HCl gas was purged for 30 min at 22° C. Reaction mixture was concentrated under reduced pressure and basified with NH₄OH solution under cooling. Product was extracted with dichloromethane, washed with brine, dried over anhydrous Na₂SO₄ and concentrated under reduced pressure to furnish title compound as a color less thick liquid.

Yield=4.4 g (88%). TLC (50% ethyl acetate in Hexane): Rf=0.35),

LCMS: Rt_{H8}=0.118 [M+H]⁺=298.0, 299.9,

¹H NMR (300 MHz, CDCl₃) δ 7.68-7.59 (m, 1H), 7.48-7.39 (m, 1H), 6.99 (dd, 1H, J=9 Hz, 4.5 Hz), 4.1-3.69 (m, 3H), 1.8 (s, 3H).

### h) N-(2-(5-bromo-2-fluorophenyl)-3,3-difluoro-4-hydroxybutan-2-yl)-2-chloroacetamide

To an ice cooled solution of 3-amino-3-(5-bromo-2-fluorophenyl)-2,2-difluorobutan-1-ol (3.1 g, 10.4 mmol), in DCM (60 ml) was added aqueous Na₂CO₃ (2.74 g, 25.8 mmol in 7.0 mL H₂O) and stirred for 10 min. Chloroacetyl chloride (0.986 ml, 11.4 mmol) was then added to the resultant reaction mixture and stirring continued for 30 min at 0° C. Upon formation of the new product by TLC analysis, K₂CO₃ (1.5 g, 10.4 mmol) in MeOH (17 mL) was added to the reaction mixture and stirred at rt for 30 min. The reaction mixture was diluted with DCM, separated the organic layer and washed successively with water and brine solution, dried over anhydrous Na₂SO₄ and concentrated under reduced pressure to furnish title compound as a colorless gum. Yield=3.3 g (78.5%).

TLC (50% ethyl acetate in Hexane): Rf=0.55),

LCMS: Rt_{H8}=1.287 [M+H]⁺=374.0, 375.9,

¹H NMR (300 MHz, CDCl₃) δ 8.28 (s, 1H), 7.53-7.38 (m, 2H), 6.92 (dd, 1H, J=11 Hz, 7.5 Hz), 4.11-3.78 (m, 2H), 2.49 (t, 1H, J=7.4 Hz), 2.08 (d, 3H).

### i) 5-(5-bromo-2-fluorophenyl)-6,6-difluoro-5-methyl-1,4-oxazepan-3-one

To a solution of t-BuOK (0.36 g, 3.2 mmol) in t-BuOH (10 mL) was added N-(2-(5-bromo-2-fluorophenyl)-3,3-difluoro-4-hydroxybutan-2-yl)-2-chloroacetamide (1.0 g, 2.6 mmol) t-BuOH (10 mL) at rt and heated to reflux temperature for 1 h 30 min. Reaction mixture was monitored by TLC analysis. Reaction mixture was concentrated under reduced pressure and adjusted pH to 2 using 2 N HCl. Ethyl acetate was added to extract the product, organic layer was washed with water, brine solution followed by drying over anhy. Na₂SO₄ and concentrated under reduced pressure. The crude compound (0.9 g) was carried forward for the next step without purification. LCMS: Rt_{H8}=1.616 [M+H]⁺=337.8, 339.9 (56%); 1.482 [M+H]⁺=675.1, 676.8 (34%).

### j) 5-(5-bromo-2-fluorophenyl)-6,6-difluoro-5-methyl-1,4-oxazepane-3-thione

To a solution of 5-(5-bromo-2-fluorophenyl)-6,6-difluoro-5-methyl-1,4-oxazepan-3-one (2.0 g, 5.93 mmol) in THF (25 mL) was added Lawesson's reagent (2.87 g, 7.1 mmol) at rt and heated to reflux temperature for 16 h. Reaction mixture was concentrated under reduced pressure and directly purified by column chromatography on silica gel using 4% ethyl acetate in Hexane to furnish title compound as colorless gum. Yield=1.0 g (50%).

LCMS: Rt_{H8}=1.75 [M+H]⁺=354.8, 355.7,

¹H NMR (300 MHz, CDCl₃) δ 7.86 (s, 1H), 7.59-7.41 (m, 2H), 7.02 (m, 1H), 4.81 (dt, J=3 Hz, 16 Hz), 4.55 (d, 1H), 4.1-3.95 (m, 2H), 1.92 (s, 3H).

### k) 5-(5-bromo-2-fluorophenyl)-6,6-difluoro-5-methyl-1,4-oxazepan-3-imine

A mixture of 5-(5-bromo-2-fluorophenyl)-6,6-difluoro-5-methyl-1,4-oxazepane-3-thione (1.0 g, 2.83 mmol) and 10% NH₃/MeOH (25 mL) was stirred in a sealed tube at rt for 24 h.

Reaction mixture was concentrated and purified by column chromatography on silica gel with 5% MeOH, 2% NH₃ in chloroform to furnish the title compound as a pale brown gum.

Yield=1.1 g ( ). LCMS: Rt_{H8}=0.146 [M+H]⁺=337.0, 339.0,

¹H NMR (300 MHz, DMSO-d₆) δ 7.87 (dd, 1H, J=2.5 Hz, 6.4 Hz), 7.53-7.45 (m, 1H), 7.09 (dd, 1H, J=5.1 Hz, 9.2 Hz), 6.08 (s, 2H), 4.32-4.11 (m, 3H), 3.97-3.83 (m, 1H), 1.88 (d, 3H).

### l) tert-butyl 5-(5-bromo-2-fluorophenyl)-6,6-difluoro-5-methyl-1,4-oxazepan-3-ylidenecarbamate

To a solution of 5-(5-bromo-2-fluorophenyl)-6,6-difluoro-5-methyl-1,4-oxazepan-3-imine (1.1 g, 3.2 mmol) in dry THF (15 mL) was added diisopropyl ethyl amine (0.84 mL, 4.8 eq) at 0° C. and stirred for 15 min. di-tertiarybutyl pyrocarbonate (0.98 mL, 4.2 eq) was added to the reaction mixture and stirred 2 h. Reaction mixture was concentrated and the crude product was purified by column chromatography on silica gel with 8% ethyl acetate in Hexane. Yield=950 mg (67%). TLC (20% ethyl acetate in Hexane): Rf=0.75),

LCMS: Rt_{H8}=1.781 [M+H-Boc]⁺=337.0, 339.0,

¹H NMR (300 MHz, CDCl₃) δ 10.9 (s, 1H), 7.61-7.39 (m, 2H), 7.03-6.95 (m, 1H), 4.42-4.21 (m, 2H), 4.03-3.81 (m, 2H), 1.93 (s, 3H), 1.51 (s, 9H).

### m) tert-butyl 5-(5-azido-2-fluorophenyl)-6,6-difluoro-5-methyl-2,5,6,7-tetrahydro-1,4-oxazepin-3-ylcarbamate

To a solution of tert-butyl 5-(5-bromo-2-fluorophenyl)-6,6-difluoro-5-methyl-1,4-oxazepan-3-ylidenecarbamate (1.72 g, 3.94 mmol) and trans-N,N′-dimethylcyclohexane-1,2-diamine (0.62 mL, 3.94 mmol), in ethanol (60 mL) was added a solution of NaN₃ (2.05 g, 31.5 mmol), (+)-sodium-L-ascorbate (0.312 g, 1.57 mmol) in water (16 mL). The reaction mixture was degassed with argon for 15 min. Cu(I) (0.3 g, 1.57 mmol) was added to the reaction mixture and heated to 70° C. for 5 min.

The reaction mixture was concentrated under reduced pressure, diluted with ethyl acetate, washed with brine and organic layer was dried over anhydrous sodium sulphate and concentrated under reduced pressure. The crude reaction mass was purified by column chromatography on silica gel with 8%-40% ethyl acetate in hexane to furnish title compound along with the corresponding amine. Yield=Amine (0.62 g, 36%); Azide (0.32 g, 22%). TLC (10% ethyl acetate in Hexane for azide): Rf=0.5; (50% ethyl acetate in Hexane for amine; Rf=0.4). The azide (620 mg, 1.5 mmol) was hydrogenated with H₂ gas under balloon pressure in the presence of 10% Pd/C (50 mg) in ethyl acetate (10 mL) for 1 h at rt. Catalyst was filtered using a short bed of celite and the filtrate was concentrated under reduced pressure to furnish amine product as color less gum. Yield=575 mg, (99%).

Azide: LCMS: Rt_{H8}=1.683 [M+H]⁺=399.9,

¹H NMR (400 MHz, CDCl₃) δ 10.89 (s, 1H), 7.21-6.95 (m, 3H), 4.41-4.22 (m, 2H), 4.05-3.80 (m, 2H), 1.98 (s, 3H), 1.25 (s, 9H);

Amine: LCMS: Rt_{H8}=0.38 [M+H-Boc]⁺=374.2, 274.2,

¹H NMR (400 MHz, CDCl₃) δ 10.62 (s, 1H), 6.91-6.83 (m, 1H), 6.72-6.6.67 (m, 1H), 6.64-6.59 (m, 1H), 4.41-4.4.15 (m, 2H), 4.03-3.85 (m, 4H), 1.90 (s, 3H), 1.49 (s, 9H).

### n) tert-butyl 5-(5-(5-chloropicolinamido)-2-fluorophenyl)-6,6-difluoro-5-methyl-2,5,6,7-tetrahydro-1,4-oxazepin-3-ylcarbamate

To a solution of 5-Chloro-pyridine-2-carboxylic acid (0.085 g, 0.54 mmol) in dry DMF (3.0 mL), Et₃N (0.22 mL, 1.6 mmol) and EDCl (0.128 mg, 0.81 mmol) and HOAt (0.11 g, 0.81 mmol) and tert-butyl 5-(5-amino-2-fluorophenyl)-6,6-difluoro-5-methyl-2,5,6,7-tetrahydro-1,4-oxazepin-3-ylcarbamate (0.201 g, 0.54 mmol) were added and stirred at rt for 24 h. upon completion of the reaction, reaction mixture was poured into a rapidly stirred ice cold water to obtain precipitate. Yield=220 mg, (80%). TLC (30% ethyl acetate in Hexane): Rf=0.4,

LCMS: Rt_{H8}=1.78 [M+H-Boc]⁺=413.0, 414.8,

¹H NMR (400 MHz, CDCl₃) δ 10.89 (s, 1H), 9.91 (s, 1H), 8.6 (s, 1H), 8.22 (d, 1H, J=9.3 Hz), 7.90 (dd, 2H, J=10.2 Hz, 3.1 Hz), 7.69 (d, 1H), 7.11 (t, 1H), 4.44-4.21 (m, 2H), 3.93-4.18 (m, 2H), 1.98 (s, 3H), 1.49 (s, 9H).

### o) N-(3-(3-amino-6,6-difluoro-5-methyl-2,5,6,7-tetrahydro-1,4-oxazepin-5-yl)-4-fluorophenyl)-5-chloropicolinamide

A solution of tert-butyl 5-(5-(5-chloropicolinamido)-2-fluorophenyl)-6,6-difluoro-5-methyl-2,5,6,7-tetrahydro-1,4-oxazepin-3-ylcarbamate (0.210 g, 0.41 mmol) in 10% dioxane in HCl was heated in a sealed tube at 55° C. for 5 h. Reaction mixture was concentrated under reduced pressure, basified with 2% methanolic ammonia and purified by column chromatography on silica gel with MeOH/DCM (3:97) to obtain the title compound as an off white solid. Yield=0.08 g (50%). TLC (20% methanol in chloroform): Rf=0.35, m.p.=190-193° C.,

LCMS: Rt_{H8}=0.39 [M+H]⁺=412.8, 415.0,

¹H NMR (400 MHz, DMSO-d₆) δ 8.79 (d, 1H), 8.23-8.12 (m, 2H), 8.07-8.02 (dd, 1H, J=9.6 Hz, 3.4 Hz), 7.85 (dt, 1H, J=8.5 Hz, 2.6 Hz), 7.10 (dd, 1H, J=12.2 Hz, 7.6 Hz), 5.98 (s, 2H), 4.29-4.08 (m, 3H), 3.98-3.85 (m, 1H), 1.76 (s, 3H).

### Example 101

The compound listed in Table 15 can be prepared by a procedure analogous to that used in example 100.

TABLE 15

| | | ¹H-NMR | MS [m/z; |
|---|---|---|---|
| Example | Compound | (δ) | (M + 1)⁺] |
| 101 | 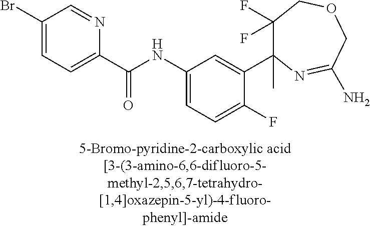 [SMILES: CC1(c2cc(NC(=O)c3ccc(Br)cn3)ccc2F)N=C(N)COCC1(F)F] | ¹H NMR (400 MHz, DMSO- d₆) δ 10.57 (s, 1H), 8.85 (d, 1H), 8.32 (d, 1H), 8.12-8.01 (m, 2H), 7.90-7.81 (m, 1H), 7.16-7.05 (m, 1H), 6.17 (s, 1H), 4.32-4.05 (m, 3H), 4.01-3.85 (m 1H), 1.76 (s, 3H) | 456, 458 |

### Examples 102 to 110

The compounds listed in Table 16 were prepared by a procedure analogous to that used in Example 34

TABLE 16

| | | | MS |
|---|---|---|---|
| | | ¹H-NMR | [m/z; |
| Example | Compound | (δ; DMSO-d₆) | (M + 1)⁺] |
| 102 | 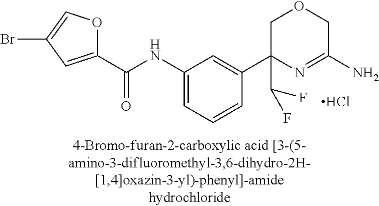 [SMILES: NC1=NC(c2cccc(NC(=O)c3cc(Br)co3)c2)(C(F)F)COC1 ; Cl] | 11.09-10.96 (m, 1 H), 10.50 (s, 1 H), 9.65 (d, 1 H), 8.67 (d, 1 H), 8.25 (s, 1 H), 7.91 (s, 1 H), 7.77 (d, 1 H), 7.57 (s, 1 H), 7.52-7.43 (m, 1 H), 7.30 (d, 1 H), 6.71 (t, 1 H, CHF2), 4.73-4.57 (m, 2 H), 4.37 (d, 1 H), 4.04 (d, 1 H) | 414, 416 |
| 103 | 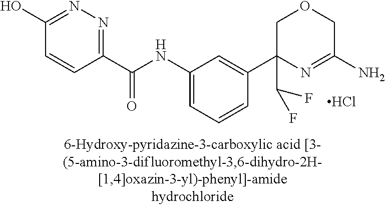 [SMILES: NC1=NC(c2cccc(NC(=O)c3ccc(O)nn3)c2)(C(F)F)COC1 ; Cl] | 10.15 (s, 1 H), 7.90 (s, 1 H), 7.83 (s, 1 H), 7.49 (d, 1 H), 7.31-7.16 (m, 2 H), 6.06-5.76 (m, 5 H), 4.08 (d, 1 H), 4.04-3.96 (m, 1 H), 3.95-3.87 (m, 1 H), 3.71 (d, 1 H) | 364 |
| 104 | 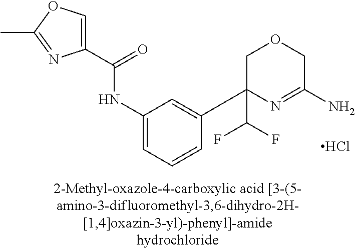 [SMILES: Cc1nc(C(=O)Nc2cccc(C3(C(F)F)COCC(N)=N3)c2)co1 ; Cl] | 11.09 (s, 1 H), 10.24 (s, 1 H), 9.71 (s, 1 H), 8.83 (s, 1 H), 8.67 (s, 1 H), 7.96 (s, 1 H), 7.90 (d, 1 H), 7.46 (t, 1 H), 7.30 (d, 1 H), 6.69 (t, 1 H, CHF2), 4.72-4.55 (m, 2 H), 4.38 (d, 1 H), 4.03 (d, 1 H), 2.52 (s, 3 H) | 351 |
| 105 | 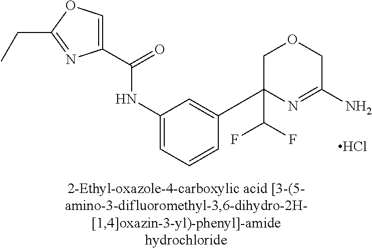 [SMILES: CCc1nc(C(=O)Nc2cccc(C3(C(F)F)COCC(N)=N3)c2)co1 ; Cl] | 11.06 (s, 1 H), 10.18 (s, 1 H), 9.68 (s, 1 H), 8.77 (s, 1 H), 8.69 (s, 1 H), 8.00-7.83 (m, 2 H), 7.46 (t, 1 H), 7.30 (d, 1 H), 6.70 (t, 1 H, CHF2), 4.73- 4.56 (m, 2 H), 4.38 (d, 1 H), 4.03 (d, 1 H), 2.86 (q, 2 H), 1.30 (t, 3 H) | 365 |
| 106 | 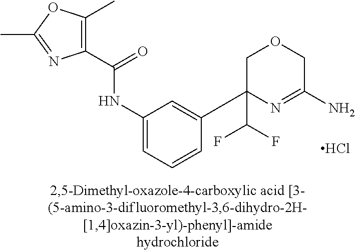 [SMILES: Cc1nc(C(=O)Nc2cccc(C3(C(F)F)COCC(N)=N3)c2)c(C)o1 ; Cl] | 11.05 (s, 1 H), 10.04 (s, 1 H), 9.69 (s, 1 H), 8.79 (s, 1 H), 7.95 (s, 1 H), 7.90 (d, 1 H), 7.44 (t, 1 H), 7.28 (d, 1 H), 6.69 (t, 1 H, CHF2), 4.72-4.57 (m, 2 H), 4.38 (d, 1 H), 4.02 (d, 1 H), 2.59 (s, 3 H), 2.46 (s, 3 H) | 365 |
| 107 | 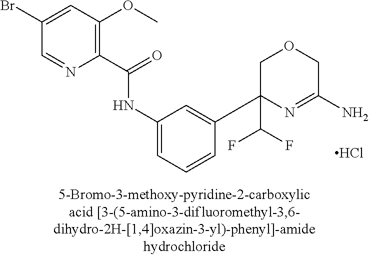 [SMILES: COc1cc(Br)cnc1C(=O)Nc1cccc(C2(C(F)F)COCC(N)=N2)c1 ; Cl] | 11.11 (s, 1 H), 10.61 (s, 1 H), 9.72 (s, 1 H), 8.84 (s, 1 H), 8.39-8.27 (m, 1 H), 7.98 (s, 1 H), 7.90 (s, 1 H), 7.77 (d, 1 H), 7.47 (t, 1 H), 7.31 (d, 1 H), 6.71 (t, 1 H, CHF2), 4.72-4.55 (m, 2 H), 4.38 (d, 1 H), 4.04 (d, 1 H), 3.89 (s, 3 H) | 455, 457 |
| 108 | 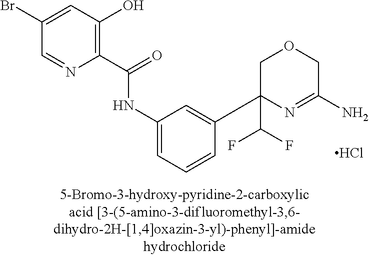 [SMILES: NC1=NC(c2cccc(NC(=O)c3ncc(Br)cc3O)c2)(C(F)F)COC1 ; Cl] | 12.20 (br. s., 1 H), 11.13 (s, 1 H), 11.02 (s, 1 H), 9.72 (s, 1 H), 8.85 (s, 1 H), 8.37 (s, 1 H), 8.00-7.84 (m, 3 H), 7.53 (t, 1 H), 7.39 (d, 1 H), 6.71 (t, 1 H, CHF2), 4.74- 4.54 (m, 2 H), 4.40 (d, 1 H), 4.06 (d, 1 H) | 441, 443 |
| 109 | 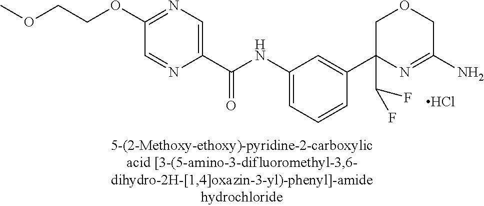 [SMILES: COCCOc1cnc(C(=O)Nc2cccc(C3(C(F)F)COCC(N)=N3)c2)cn1 ; Cl] | 10.40 (s, 1 H), 8.87 (s, 1 H), 8.44 (s, 1 H), 8.00 (br. s., 1 H), 7.81 (br. s., 1 H), 7.32 (d, 2 H), 6.06-5.88 (m, 3 H), 4.53 (dd, 2 H), 4.08-3.97 (m, 2 H), 3.97-3.84 (m, 1 H), 3.65-3.80 (m, 3 H), 3.31 (s, 3 H) | 422 |
| 110 | 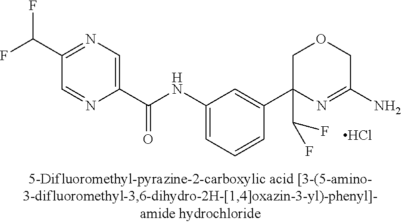 [SMILES: NC1=NC(c2cccc(NC(=O)c3cnc(C(F)F)cn3)c2)(C(F)F)COC1 ; Cl] | 11.07 (s, 1 H), 11.01 (s, 1 H), 9.68 (s, 1 H), 9.41 (s, 1 H), 9.11 (s, 1 H), 8.74 (br. s., 1 H), 8.08 (br. s., 1 H), 7.99 (d, 1 H), 7.53 (t, 1 H), 7.36 (s, 1 H), 7.27 (t, 1 H, CHF2), 6.72 (t, 1 H, CHF2), 4.73-4.55 (m, 2 H), 4.39 (d, 1 H), 4.06 (d, 1 H) | 398 |

### Examples 111 to 151

The compounds listed in Table 17 were prepared by procedures analogous to those used in Examples 42 or 112.

For enantiomerically pure compounds the racemic precursor [5-(5-amino-2-fluoro-phenyl)-5-difluoromethyl-5,6-dihydro-2H-[1,4]oxazin-3-yl]-carbamic acid tert-butyl ester (example 42j)) was separated via prep-HPLC on Chiralpak AD-H 250×4.6 mm column using supercritical CO2/EtOH 9:1 as an eluent. The desired compound was the slower eluting (R)-enantiomer. Enantiomeric excess=99.7%; =−109.7° (c=1, CHCl₃).

TABLE 17

| | | | MS |
|---|---|---|---|
| | | ¹H-NMR | [m/z; |
| Example | Compound | (δ; DMSO-d₆) | (M + 1)⁺] |
| 111 | 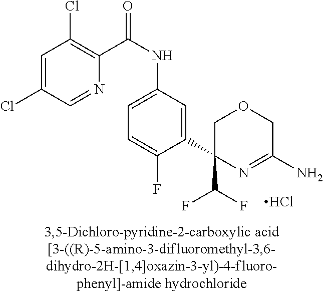 [SMILES: NC1=N[C@@](c2cc(NC(=O)c3ncc(Cl)cc3Cl)ccc2F)(C(F)F)COC1 ; Cl] | 11.06 (s, 1H), 10.99 (s, 1H), 9.76 (s, 1H), 8.75 (s, 1H), 8.73 (s 1H), 8.48 (d, 1H), 7.93-7.86 (m, 2H), 7.41 (t, 1H), 6.79 (t, J = 54 Hz, 1H), 4.71 (d, 1H), 4.65 (d, 1H), 4.34 (d, 1H), 4.18 (d, 1H) | 433 |
| 112 | 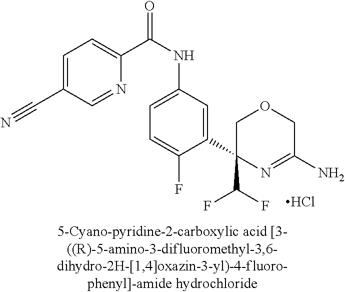 [SMILES: N#Cc1ccc(C(=O)Nc2ccc(F)c([C@]3(C(F)F)COCC(N)=N3)c2)nc1 ; Cl] | 11.09 (s, 1H), 11.01 (s, 1H), 9.76 (s, 1H), 9.22 (s, 1H), 8.75 (s, 1H), 8.61 (d, 1H), 8.30 (d, 1H), 8.12- 8.06 (m, 2H), 7.41 (dd, 1H), 6.79 (t, J = 54 Hz, 1H), 4.71 (d, 1H), 4.65 (d, 1H), 4.34 (d, 1H), 4.18 (d, 1H) | 390 |
| 113 | 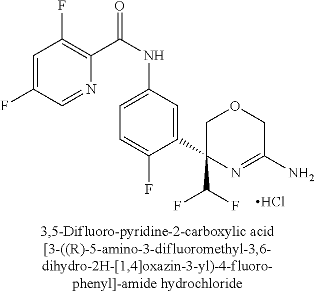 [SMILES: NC1=N[C@@](c2cc(NC(=O)c3ncc(F)cc3F)ccc2F)(C(F)F)COC1 ; Cl] | 11.00 (s, 1H), 10.85 (s, 1H) 9.74 (s, 1H), 8.72- 8.67 (m, 2H), 8.23-8.18 (m, 1H), 8.04-8.00 (m, 1H), 7.96-7.92 (m, 1H), 7.40 (dd, 1H), 6.79 (t, J = 54 Hz, 1H), 4.71 (d, 1H), 4.65 (d, 1H), 4.34 (d, 1H), 4.18 (d, 1H) | 401 |
| 114 | 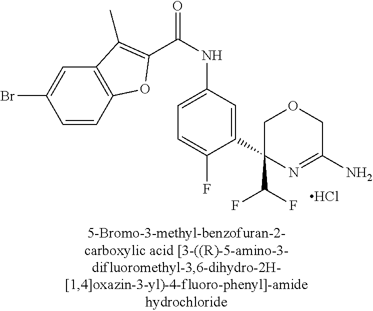 [SMILES: Cc1c(C(=O)Nc2ccc(F)c([C@]3(C(F)F)COCC(N)=N3)c2)oc2ccc(Br)cc12 ; Cl] | 11.01 (s, 1H), 10.74 (s, 1H), 9.76 (s, 1H), 8.74 (s, 1H), 8.08 (s, 1H), 8.03- 7.99 (m, 1H), 7.98-7.93 (m, 1H), 7.69-7.64 (m, 2H), 7.39 (dd, 1H), 6.79 (t, J = 54 Hz, 1H), 4.71 (d, 1H), 4.65 (d, 1H), 4.34 (d, 1H), 4.18 (d, 1H), 2.57 (s, 3H) | 496, 498 |
| 115 | 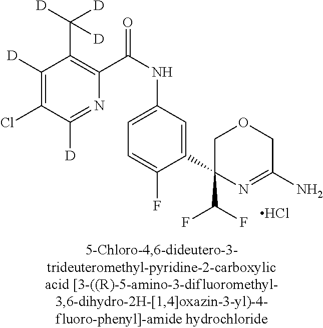 [SMILES: [2H]c1nc(C(=O)Nc2ccc(F)c([C@]3(C(F)F)COCC(N)=N3)c2)c(C([2H])([2H])[2H])c([2H])c1Cl ; Cl] | 11.02 (s, 1H), 10.79 (s, 1H) 9.77 (s, 1H), 8.76 (s, 1H), 8.01-7.94 (m, 2H), 7.38 (dd, 1H), 6.78 (t, J = 54 Hz, 1H), 4.71 (d, 1H), 4.65 (d, 1H), 4.34 (d, 1H), 4.18 (d, 1H) | 418 |
| 116 | 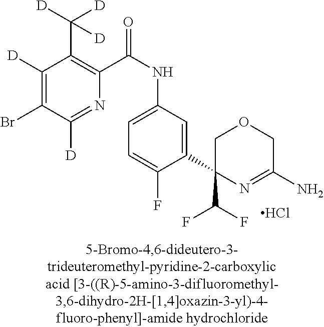 [SMILES: [2H]c1nc(C(=O)Nc2ccc(F)c([C@]3(C(F)F)COCC(N)=N3)c2)c(C([2H])([2H])[2H])c([2H])c1Br ; Cl] | 11.00 (s, 1H), 10.79 (s, 1H) 9.76 (s, 1H), 8.73 (s, 1H), 8.01-7.94 (m, 2H), 7.38 (dd, 1H), 6.78 (t, J = 54 Hz, 1H), 4.71 (d, 1H), 4.65 (d, 1H), 4.34 (d, 1H), 4.18 (d, 1H) | 462, 464 |
| 117 | 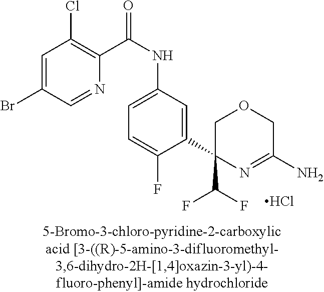 [SMILES: NC1=N[C@@](c2cc(NC(=O)c3ncc(Br)cc3Cl)ccc2F)(C(F)F)COC1 ; Cl] | 11.08 (s, 1H), 10.99 (s, 1H), 9.78 (s, 1H), 8.81 (s, 1H), 8.77 (s, 1H), 8.58 (s, 1H), 7.92-7.85 (m, 2H), 7.40 (dd, 1H), 6.79 (t, J = 54 Hz, 1H), 4.71 (d, 1H), 4.65 (d, 1H), 4.34 (d, 1H), 4.18 (d, 1H) | 478 |
| 118 | 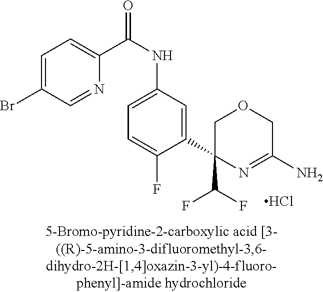 [SMILES: NC1=N[C@@](c2cc(NC(=O)c3ccc(Br)cn3)ccc2F)(C(F)F)COC1 ; Cl] | 11.08 (s, 1H), 10.99 (s, 1H), 9.78 (s, 1H), 8.81 (s, 1H), 8.77 (s, 1H), 8.35 (d, 1H), 8.12-8.05 (m, 3H), 7.39 (t, 1H), 6.78 (t, J = 54 Hz, 1H), 4.72 (d, 1H), 4.64 (d, 1H), 4.33 (d, 1H), 4.18 (d, 1H) | 443, 445 |
| 119 | 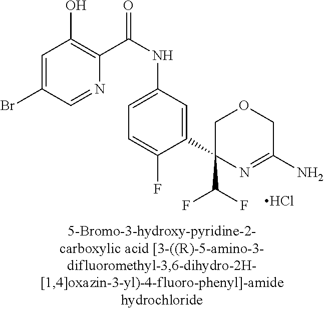 [SMILES: NC1=N[C@@](c2cc(NC(=O)c3ncc(Br)cc3O)ccc2F)(C(F)F)COC1 ; Cl] | 12.17 (s, 1H), 11.16 (s, 1H), 11.02 (s, 1H), 9.78 (s, 1H), 8.78 (s, 1H), 8.37 (s, 1H), 8.03-7.96 (m, 2H), 7.90 (s, 1H), 7.42 (t, 1H), 6.78 (t, J = 54 Hz, 1H), 4.72 (d, 1H), 4.64 (d, 1H), 4.33 (d, 1H), 4.18 (d, 1H) | 459, 461 |
| 120 | 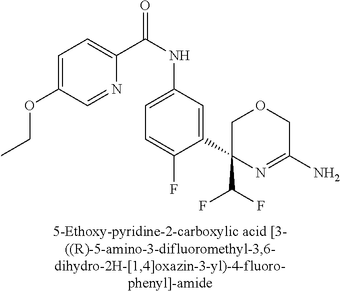 [SMILES: CCOc1ccc(C(=O)Nc2ccc(F)c([C@]3(C(F)F)COCC(N)=N3)c2)nc1] | 10.42 (s, 1H), 8.37 (s, 1H), 8.13-8.08 (m, 2H), 7.86-7.80 (m, 1H), 7.59 (d, 1H), 7.16 (t, 1H), 6.16 (s, 2H), 6.14 (t, J = 54 Hz, 1H), 4.21 (q, 2H), 4.12 (d, 1H), 4.01 (d, 1H), 3.90 (d, 1H), 3.82 (d, 1H), 1.38 (t, 3H) | 409 |
| 121 | 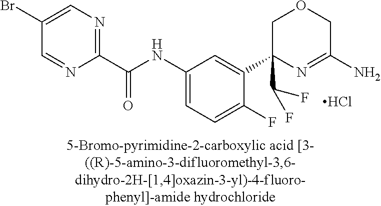 [SMILES: NC1=N[C@@](c2cc(NC(=O)c3ncc(Br)cn3)ccc2F)(C(F)F)COC1 ; Cl] | 11.17-11.00 (m, 2H), 9.79 (br. s., 1 H), 9.24 (s, 2 H), 8.80 (br. s., 1 H), 8.15-7.98 (m, 2 H), 7.40 (dd, 1 H), 6.79 (t, 1 H, CHF2), 4.82-4.68 (m, 1 H), 4.68-4.55 (m, 1 H), 4.33 (d, 1 H), 4.18 (d, 1 H) | 488, 490 |
| 122 | 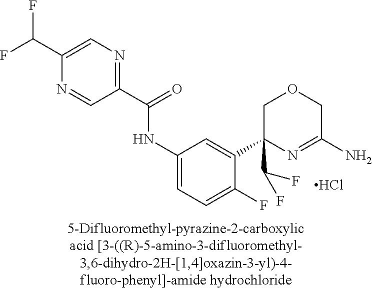 [SMILES: NC1=N[C@@](c2cc(NC(=O)c3cnc(C(F)F)cn3)ccc2F)(C(F)F)COC1 ; Cl] | 11.15 (s, 1 H), 11.06 (s, 1 H), 9.80 (s, 1 H), 9.41 (s, 1 H), 9.11 (s, 1 H), 8.81 (br. s., 1 H), 8.19-8.01 (m, 2 H), 7.42 (dd, 1 H), 7.32-7.21 (m, 1 H), 7.21- 7.09 (m, 1 H), 6.79 (t, 1 H, CHF2), 4.78-4.69 (m, 1 H), 4.69-4.58 (m, 1 H), 4.33 (d, 1 H), 4.19 (d, 1 H) | 416 |
| 123 | 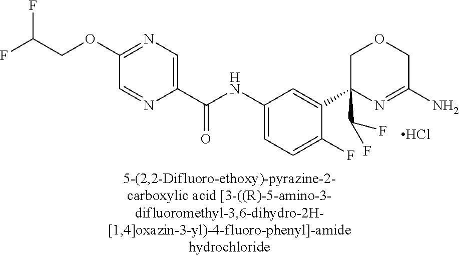 [SMILES: NC1=N[C@@](c2cc(NC(=O)c3cnc(OCC(F)F)cn3)ccc2F)(C(F)F)COC1 ; Cl] | 11.05 (s, 1 H), 10.82 (s, 1 H), 9.82 (br. s., 1 H), 8.92 (s, 1 H), 8.85 (br. s., 1 H), 8.56 (s, 1 H), 8.14-7.99 (m, 2 H), 7.39 (dd, 1 H), 7.24 (t, 1 H), 7.19-7.10 (m, 1 H), 6.78 (t, 1 H, CHF2), 6.57-6.38 (m, 1 H), 4.81-4.68 (m, 3 H), 4.68-4.58 (m, 1 H), 4.33 (d, 1 H), 4.18 (d, 1 H) | 446 |
| 124 | 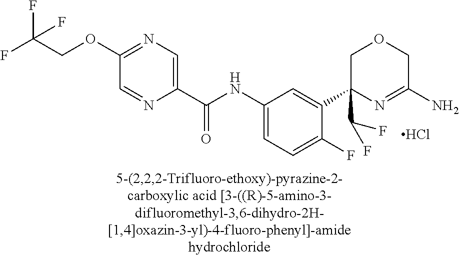 [SMILES: NC1=N[C@@](c2cc(NC(=O)c3cnc(OCC(F)(F)F)cn3)ccc2F)(C(F)F)COC1 ; Cl] | 10.97 (s, 1 H), 10.86 (s, 1 H), 9.72 (br. s., 1 H), 8.94 (s, 1 H), 8.68 (br. s., 1 H), 8.64 (s, 1 H), 8.15-8.07 (m, 1 H), 8.04 (d, 1 H), 7.39 (dd, 1 H), 6.79 (t, 1 H, CHF2), 5.17 (q, 2 H), 4.76-4.68 (m, 1 H), 4.68- 4.59 (m, 1 H), 4.33 (d, 1 H), 4.18 (d, 1 H) | 464 |
| 125 | 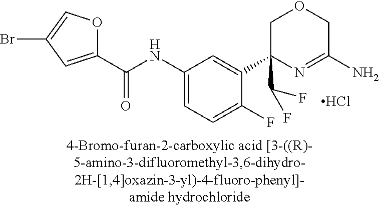 [SMILES: NC1=N[C@@](c2cc(NC(=O)c3cc(Br)co3)ccc2F)(C(F)F)COC1 ; Cl] | 11.04 (s, 1 H), 10.64 (s, 1 H), 9.78 (s, 1 H), 8.79 (s, 1 H), 8.25 (s, 1 H), 7.95 (dd, 1 H), 7.86 (dd, 1 H), 7.60 (s, 1 H), 7.37 (dd, 1 H), 6.77 (t, 1 H, CHF2), 4.76-4.68 (m, 1 H), 4.68- 4.59 (m, 1 H), 4.32 (d, 1 H), 4.17 (d, 1 H) | 432, 434 |
| 126 | 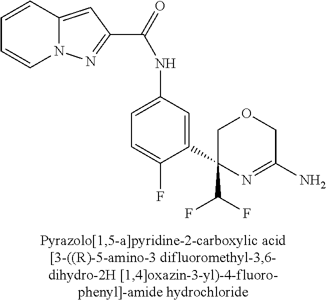 [SMILES: NC1=N[C@@](c2cc(NC(=O)c3cc4ccccn4n3)ccc2F)(C(F)F)COC1] | 11.00 (s, 1 H), 10.65 (s, 1 H), 9.77 (s, 1 H), 8.78 (s, 1 H), 8.73 (d, 1 H), 8.07 (d, 1 H), 7.99 (d, 1 H), 7.82 (d, 1 H), 7.33 (m, 2 H), 7.13 (s, 1 H), 7.08 (t, 1 H), 6.77 (t, 1H, CHF₂), 4.71 (d, 1H), 4.64 (d, 1H), 4.32 (d, 1H), 4.17 (d, 1H) | 404 |
| 127 | 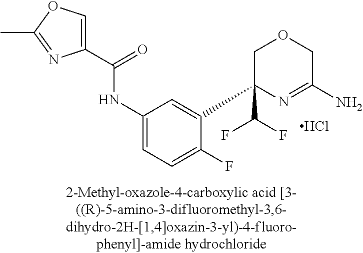 [SMILES: Cc1nc(C(=O)Nc2ccc(F)c([C@]3(C(F)F)COCC(N)=N3)c2)co1 ; Cl] | 11.01 (s, 1 H), 10.41 (s, 1 H), 9.77 (s, 1 H), 8.77 (s, 1 H), 8.68 (s, 1 H), 8.08- 7.99 (m, 1 H), 7.99-7.89 (m, 1 H), 7.35 (dd, 1 H), 6.77 (t, 1 H, CHF2), 4.76- 4.67 (m, 1 H), 4.67-4.57 (m, 1 H), 4.31 (d, 1 H), 4.16 (d, 1 H), 2.52 (s, 3 H) | 369 |
| 128 | 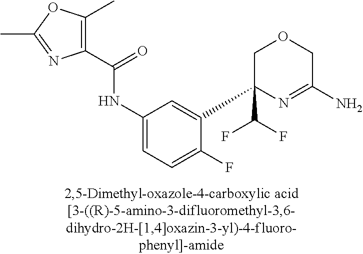 [SMILES: Cc1nc(C(=O)Nc2ccc(F)c([C@]3(C(F)F)COCC(N)=N3)c2)c(C)o1] | 10.97 (s, 1 H), 10.22 (s, 1 H), 9.76 (s, 1 H), 8.75 (br. s., 1 H), 8.05-7.90 (m, 2 H), 7.33 (dd, 1 H), 6.76 (t, 1 H, CHF2), 4.77-4.67 (m, 1 H), 4.67-4.58 (m, 1 H), 4.32 (d, 1 H), 4.15 (d, 1 H), 2.58 (s, 3 H), 2.46 (s, 3 H) | 383 |
| 129 | 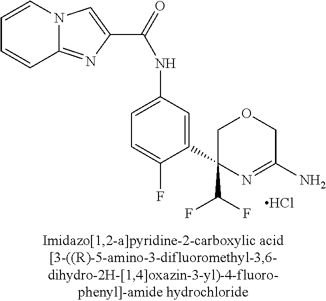 [SMILES: NC1=N[C@@](c2cc(NC(=O)c3cn4ccccc4n3)ccc2F)(C(F)F)COC1 ; Cl] | 11.07 (s, 1 H), 10.80 (s, 1 H), 9.85 (s, 1 H), 8.90 (s, 1 H), 8.49 (d, 1 H), 8.39 (d, 1 H), 7.87 (m, 2 H), 7.63 (d, 1 H), 7.41 (m, 1 H), 7.14 (dd, 1 H), 6.99 (t, 1 H), 6.25 (t, 1H, CHF₂), 4.14 (m, 3 H), 3.96 (d, 1H) | 404 |
| 130 | 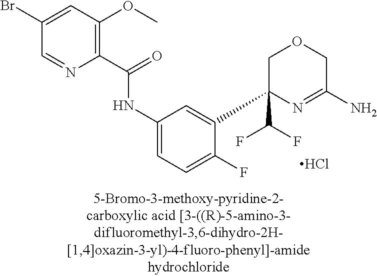 [SMILES: COc1cc(Br)cnc1C(=O)Nc1ccc(F)c([C@]2(C(F)F)COCC(N)=N2)c1 ; Cl] | 11.04 (s, 1 H), 10.71 (s, 1 H), 9.76 (s, 1 H), 8.75 (s, 1 H), 8.36 (s, 1 H), 7.99 (s, 1 H), 7.94 (d, 1 H), 7.86-7.84 (m, 1 H), 7.37 (dd, 1 H), 6.78 (t, 1 H, CHF2), 4.68 (q, 2 H), 4.33 (d, 1 H), 4.17 (d, 1 H), 3.89 (s, 3 H) | 473, 475 |
| 131 | 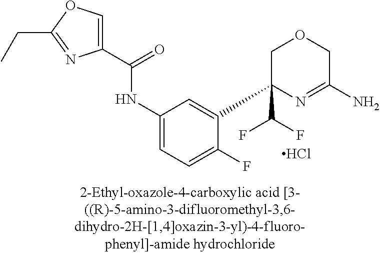 [SMILES: CCc1nc(C(=O)Nc2ccc(F)c([C@]3(C(F)F)COCC(N)=N3)c2)co1 ; Cl] | 11.01 (s, 1 H), 10.35 (s, 1 H), 9.77 (s, 1 H), 8.83- 8.73 (m, 1 H), 8.70 (s, 1 H), 8.06-7.90 (m, 2 H), 7.35 (dd, 1 H), 6.77 (t, 1 H, CHF2), 4.76-4.67 (m, 1 H), 4.67-4.57 (m, 1 H), 4.32 (d, 1 H), 4.17 (d, 1 H), 2.86 (q, 2 H), 1.30 (t, 3 H) | 383 |
| 132 | 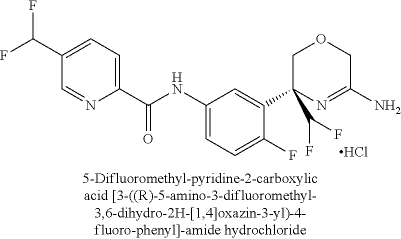 [SMILES: NC1=N[C@@](c2cc(NC(=O)c3ccc(C(F)F)cn3)ccc2F)(C(F)F)COC1 ; Cl] | 11.03 (s, 1H), 10.97 (s, 1H), 9.71 (s, 1H), 8.95 (s, 1H), 8.66 (br. s., 1H), 8.31-8.27 (m, 2H), 8.14- 8.07 (m, 2H), 7.45-7.38 (m, 1H), 7.28 (t, 1H), 6.79 (t, 1H), 4.71 (d, 1H), 4.63 (d, 1H), 4.33 (d, 1H), 4.18 (d, 1H) | 415 |
| 133 | 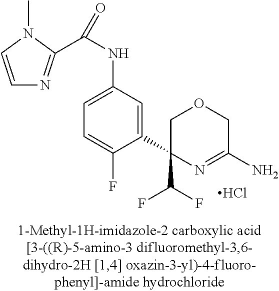 [SMILES: Cn1ccnc1C(=O)Nc1ccc(F)c([C@]2(C(F)F)COCC(N)=N2)c1 ; Cl] | 11.04 (s, 1 H), 10.77 (s, 1 H), 9.82 (s, 1 H), 8.88 (s, 1 H), 7.97 (d, 1 H), 7.54 (d, 1 H), 7.35 (t, 1 H), 7.23 (s, 1 H), 6.75 (t, 1H, CHF₂), 4.70 (d, 1H), 4.62 (d, 1H), 4.31 (d, 1H), 4.16 (d, 1H) | 368 |
| 134 | 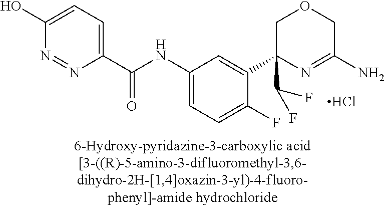 [SMILES: NC1=N[C@@](c2cc(NC(=O)c3ccc(O)nn3)ccc2F)(C(F)F)COC1 ; Cl] | 13.61 (s, 1 H), 11.04 (s, 1 H), 10.57 (s, 1 H), 9.80 (s, 1 H), 8.88-8.74 (m, 1 H), 8.01-7.83 (m, 3 H), 7.37 (dd, 1 H), 7.03 (dd, 1 H), 6.77 (t, 1 H, CHF2), 4.79- 4.67 (m, 1 H), 4.67-4.58 (m, 1 H), 4.32 (d, 1 H), 4.17 (d, 1 H) | 382 |
| 135 | 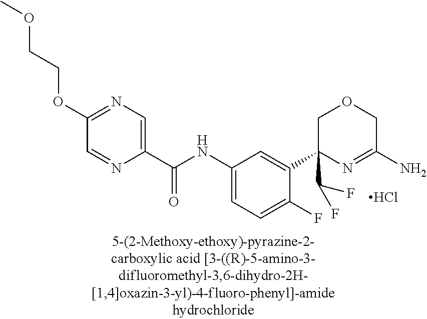 [SMILES: COCCOc1cnc(C(=O)Nc2ccc(F)c([C@]3(C(F)F)COCC(N)=N3)c2)cn1 ; Cl] | 11.01 (s, 1 H), 10.77 (s, 1 H), 9.77 (br. s., 1 H), 8.92 (d, 1 H), 8.77 (br. s., 1 H), 8.48 (d, 1 H), 8.17-7.98 (m, 2 H), 7.40 (dd, 1 H), 6.80 (t, 1 H, CHF2), 4.83- 4.60 (m, 2 H), 4.60-4.51 (m, 2 H), 4.36 (d, 1 H), 4.20 (d, 1 H), 3.75 (dd, 2 H), 3.34 (s, 3 H) | 440 |
| 136 | 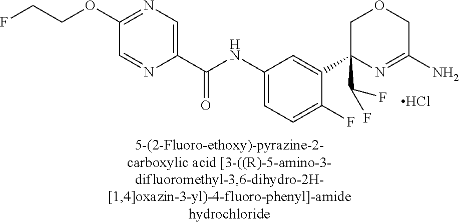 [SMILES: NC1=N[C@@](c2cc(NC(=O)c3cnc(OCCF)cn3)ccc2F)(C(F)F)COC1 ; Cl] | 10.52 (br. s., 1 H), 8.87 (s, 1 H), 8.48 (s, 1 H), 8.13 (d, 1 H), 7.80 (br. s., 1 H), 7.17 (br. s., 1 H), 6.23- 6.05 (m, 2 H), 4.92-4.82 (m, 1 H), 4.82-4.73 (m, 1 H), 4.73-4.67 (m, 1 H), 4.67-4.57 (m, 1 H), 4.11 (d, 1 H), 4.06-3.96 (m, 1 H), 3.91 (d, 1 H), 3.84 (d, 1 H) | 428 |
| 137 | 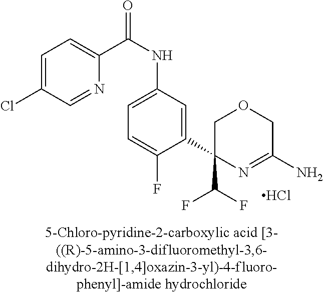 [SMILES: NC1=N[C@@](c2cc(NC(=O)c3ccc(Cl)cn3)ccc2F)(C(F)F)COC1 ; Cl] | 11.01 (s, 1H), 10.93 (s, 1H), 9.77 (s, 1H), 8.80 (s, 1H), 8.75 (s, 1H), 8.22 (d, 1H), 8.16 (d, 1H), 8.11- 8.04 (m, 2H), 7.38 (t, 1H), 6.78 (t, J = 54 Hz, 1H), 4.72 (d, 1H), 4.64 (d, 1H), 4.33 (d, 1H), 4.18 (d, 1H) | 399 |
| 138 | 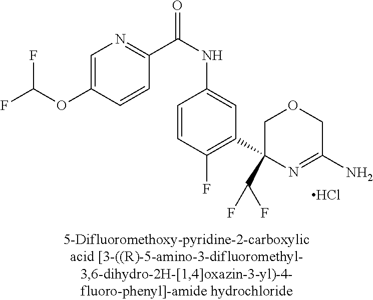 [SMILES: NC1=N[C@@](c2cc(NC(=O)c3ccc(OC(F)F)cn3)ccc2F)(C(F)F)COC1 ; Cl] | 11.02 (br. s., 1H), 10.89 (s, 1H), 9.78 (br. s., 1H), 8.77 (br. s., 1 H), 8.63 (d, 1H), 8.24 (d, 1H), 8.05- 8.12 (m, 2H), 7.93 (dd, 1H), 7.52 (t, 1H), 7.39 (dd, 1H), 6.79 (t, 1H), 4.72 (d, 1H), 4.64 (d, 1H), 4.34 (d, 1H), 4.18 (d, 1H) | 431 |
| 139 | 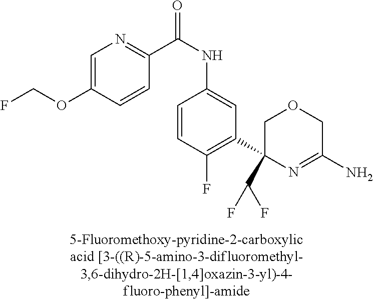 [SMILES: NC1=N[C@@](c2cc(NC(=O)c3ccc(OCF)cn3)ccc2F)(C(F)F)COC1] | 11.02 (s, 1H), 10.82 (s, 1H), 9.78 (br. s., 1H), 8.78 (br. s., 1H), 8.55 (d, 1H), 8.20 (d, 1H), 8.12- 8.05 (m, 2H), 7.83 (dd, 1H), 7.38 (dd, 1H), 6.79 (t, 1H), 6.06 (d, 2H), 4.72 (d, 1H), 4.64 (d, 1H), 4.34 (d, 1H), 4.18 (d, 1H) | 413 |
| 140 | 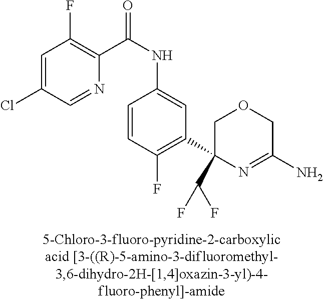 [SMILES: NC1=N[C@@](c2cc(NC(=O)c3ncc(Cl)cc3F)ccc2F)(C(F)F)COC1] | 10.96 (br. s., 1H), 10.91 (s, 1H), 9.67 (br. s., 1H), 8.68 (s, 1H), 8.59 (br. s., 1H), 8.37 (dd, 1H), 8.02 (dd, 1H), 7.97-7.90 (m, 1H), 7.40 (dd, 1H), 6.79 (t, 1H), 4.71 (d, 1H), 4.64 (d, 1H), 4.33 (d, 1H), 4.18 (d, 1H) | 417 |
| 141 | 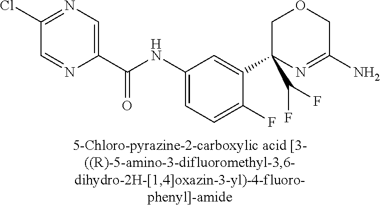 [SMILES: NC1=N[C@@](c2cc(NC(=O)c3cnc(Cl)cn3)ccc2F)(C(F)F)COC1] | 11.03 (s, 1H), 10.99 (s, 1H), 9.65 (br. s., 1H), 9.13 (s, 1H), 8.95 (s, 1H), 8.71 (br. s., 1H), 8.09 (dd, 1H), 8.06-7.96 (m, 1H), 7.40 (dd, 1H), 6.78 (t, 1H), 4.70 (d, 1H), 4.63 (d, 1H), 4.32 (d, 1H), 4.18 (d, 1H) | 400 |
| 142 | 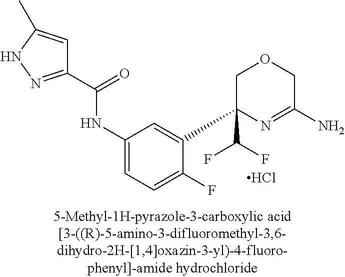 [SMILES: Cc1cc(C(=O)Nc2ccc(F)c([C@]3(C(F)F)COCC(N)=N3)c2)n[nH]1 ; Cl] | 11.02 (br. s., 1 H), 10.25 (s, 1 H), 9.81 (br. s., 1 H), 8.83 (br. s., 1 H), 8.04 (d, 1 H), 7.95 (d, 1 H), 7.33 (dd, 1 H), 6.76 (t, 1 H, CHF2), 6.54 (s, 1 H), 4.76- 4.67 (m, 1 H), 4.67-4.58 (m, 1 H), 4.33 (d, 1 H), 4.15 (d, 1 H), 2.29 (s, 3 H) | 368 |
| 143 | 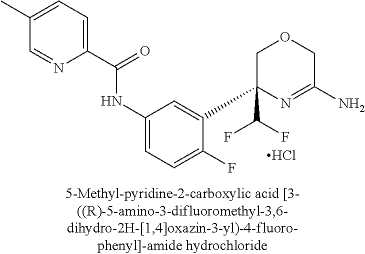 [SMILES: Cc1ccc(C(=O)Nc2ccc(F)c([C@]3(C(F)F)COCC(N)=N3)c2)nc1 ; Cl] | 11.07 (s, 1 H), 10.84 (s, 1 H), 9.86 (s, 1 H), 8.91 (br. s., 1 H), 8.59 (s, 1 H), 8.16- 8.02 (m, 3 H), 7.89 (d, 1 H), 7.37 (dd, 1 H), 6.78 (t, 1 H, CHF2), 4.78-4.68 (m, 1 H), 4.68-4.57 (m, 1 H), 4.34 (d, 1 H), 4.17 (d, 1 H), 2.43 (s, 3 H) | |
| 144 | 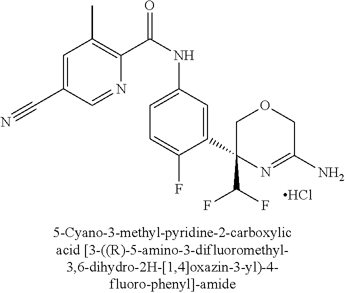 [SMILES: Cc1cc(C#N)cnc1C(=O)Nc1ccc(F)c([C@]2(C(F)F)COCC(N)=N2)c1 ; Cl] | 10.72 (s, 1H), 8.97 (s, 1H) 8.39 (s, 1H), 8.02-7.97 (m, 1H), 7.83-7.78 (m, 1H), 7.18 (dd, 1H), 6.14 (s, 2H), 6.14 (t, J = 54 Hz, 1H), 4.10 (d, 1H), 4.01 (d, 1H), 3.91 (d, 1H), 3.84 (d, 1H), 2.54 (s, 3H) | 404 |
| 145 | 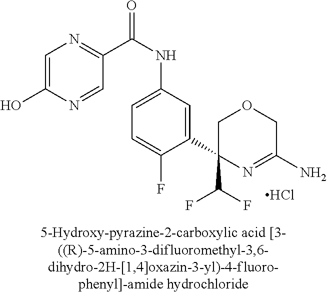 [SMILES: NC1=N[C@@](c2cc(NC(=O)c3cnc(O)cn3)ccc2F)(C(F)F)COC1 ; Cl] | 11.01 (s, 1H), 10.42 (s, 1H), 9.31 (s, 1H) 8.82 (s, 1H), 9.2-7.9 (m, 4H), 7.36 (dd, 1H), 6.29 (t, J = 54 Hz, 1H), 4.68 (m, 2H), 4.36 (d, 1H), 4.18 (d, 1H) | 382 |
| 146 | 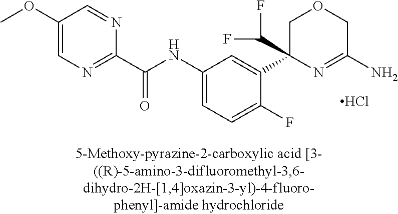 [SMILES: COc1cnc(C(=O)Nc2ccc(F)c([C@]3(C(F)F)COCC(N)=N3)c2)nc1 ; Cl] | 10.77 (s, 1H), 9.65 (s, 1H), 8.85 (s, 1H), 8.78 (s, 1H), 8.40 (s, 1H), 8.11 (d, 1H), 8.03 (m, 1H), 7.37 (dd, 1H), 6.77 (t, 1H), 4.71 (m, 2H), 4.32 (d, 1H), 4.22 (d, 1H) | 396 |
| 147 | 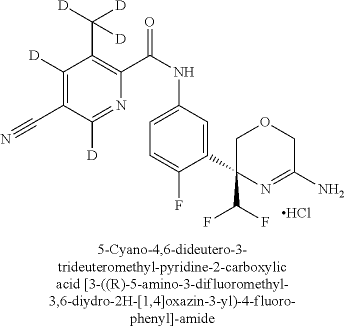 [SMILES: [2H]c1nc(C(=O)Nc2ccc(F)c([C@]3(C(F)F)COCC(N)=N3)c2)c(C([2H])([2H])[2H])c([2H])c1C#N ; Cl] | 10.72 (s, 1H), 8.02-7.97 (m, 1H), 7.83-778 (m, 1H), 7.18 (t, 1H), 6.14 (t, J = 54 Hz, 1H), 6.14 (s, 2H), 4.10 (d, 1H), 4.01 (d, 1H), 3.91 (d, 1H), 3.83 (d, 1H) | 409 |
| 148 | 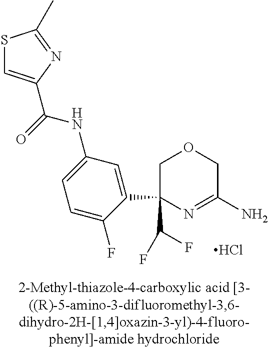 [SMILES: Cc1nc(C(=O)Nc2ccc(F)c([C@]3(C(F)F)COCC(N)=N3)c2)cs1 ; Cl] | 11.1 (s, 1H), 10.45 (s, 1H), 9.91 (s, 1H), 9.02 (s, 1H), 8.32 (s, 1H), 8.04-8.0 (m, 2H), 7.35 (t, 1H), 6.77 (t, 1H, CHF₂), 4.72 (d, 1H, AB-system), 4.65 (d, 1H, AB-system), 4.32 (d, 1H, AB-system), 4.18 (d, 1H, AB-system)), 2.77 (s, 3H) | 385 |
| 149 | 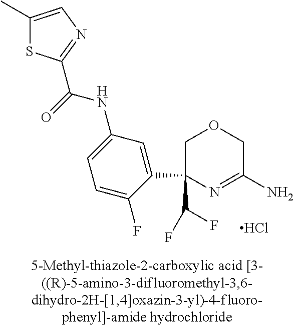 [SMILES: Cc1cnc(C(=O)Nc2ccc(F)c([C@]3(C(F)F)COCC(N)=N3)c2)s1 ; Cl] | 11.1 (s, 1H), 10.92 (s, 1H), 9.88 (s, 1H), 8.95 (s, 1H), 8.08-8.04 (m, 1H), 8.0-7.98 (m, 1H), 7.82 (s, 1H), 7.40-7.36 (m, 1H), 6.77 (t, 1H, CHF₂), 4.72 (d, 1H, AB-system), 4.65 (d, 1H, AB-system), 4.32 (d, 1H, AB-system), 4.18 (d, 1H, AB-system)), 2.58 (s, 3H) | 385 |
| 150 | 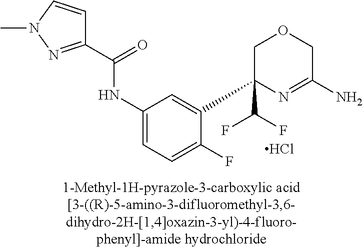 [SMILES: Cn1ccc(C(=O)Nc2ccc(F)c([C@]3(C(F)F)COCC(N)=N3)c2)n1 ; Cl] | 11.04 (s, 1 H), 10.32 (s, 1 H), 9.84 (s, 1 H), 8.87 (br. s., 1 H), 8.02 (d, 1 H), 7.96 (dd, 1 H), 7.87 (d, 1 H), 7.33 (dd, 1 H), 6.77 (t, 1 H, CHF2), 4.78-4.68 (m, 1 H), 4.68-4.58 (m, 1 H), 4.32 (d, 1 H), 4.16 (d, 1 H), 3.97 (s, 3 H) | 368 |
| 151 | 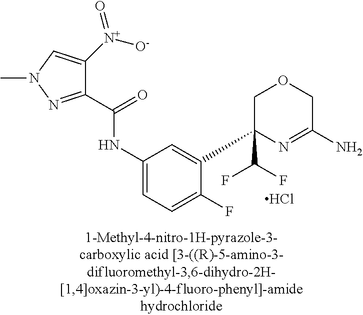 [SMILES: Cn1cc([N+](=O)[O-])c(C(=O)Nc2ccc(F)c([C@]3(C(F)F)COCC(N)=N3)c2)n1 ; Cl] | 11.08 (s, 1 H), 10.98 (s, 1 H), 9.77 (s, 1 H), 8.97 (s, 1 H), 8.76 (s, 1 H), 7.91- 7.76 (m, 2 H), 7.40 (dd, 1 H), 6.79 (t, 1 H, CHF2), 4.78-4.59 (m, 2 H), 4.33 (d, 1 H), 4.18 (d, 1 H), 3.96 (s, 3 H) | 413 |

### More Detailed Description of Preparation of Example 112

### 5-Cyano-pyridine-2-carboxylic acid [3-((R)-5-amino-3-difluoromethyl-3,6-dihydro-2H-[1,4]oxazin-3-yl)-4-fluoro-phenyl]-amide hydrochloride

### a) 5-Difluoromethyl-5-(2-fluoro-phenyl)-morpholin-3-one

5-(5-Bromo-2-fluoro-phenyl)-5-difluoromethyl-morpholin-3-one (190 g, 586 mmol) [example 42 step e)] and sodium acetate (57.7 g, 703 mmol) were suspended in 1850 mL methanol. 10% Pd on charcoal (18.7 g) was then added and the reaction mixture was shaken in a Parr apparatus in an atmosphere of hydrogen at rt. After 60 minutes the reaction mixture was filtered over celite and evaporated. The residue was dissolved in 2 L TBME and washed with aqueous NaHCO₃ and brine. The organic layer was dried over MgSO₄.H₂O and evaporated to give 143.2 g of the title compound as a white solid.

HPLC: Rt_{H1}=0.792 min; ESIMS [M+H]⁺=246;

¹H-NMR (CDCl₃, 360 MHz): 7.50-7.43 (m, 2H), 7.32-7.27 (m, 1H), 7.19 (dd, 1H), 6.62 (br, 1H), 6.37 (t, J=54 Hz, 1H), 4.34 (d, 1H), 4.31 (d, 1H), 4.22 (d, 1H), 4.20 (d, 1H).

### b) 5-Difluoromethyl-5-(2-fluoro-phenyl)-morpholine-3-thione

A mixture of 5-difluoromethyl-5-(2-fluoro-phenyl)-morpholin-3-one (141 g, 575 mmol) and Lawesson's reagent (132 g, 316 mmol) in 1400 ml of THF was heated at 68° C. for 1 h, cooled down and then evaporated. The residue was dissolved in 1 L DCM and filtered over 2 Kg silica gel with 10 L DCM to give 161 g of the title compound in the form of a greenish resin that slowly crystallized. The compound was used without further purification.

HPLC: Rt_{H1}=1.799 min; ESIMS [M+H]⁺=262; ¹H-NMR (360 MHz, CDCl₃): 7.42-7.35 (m, 1H), 7.28 (t, 1H), 7.19 (t, 1H), 7.11 (dd, 1H), 6.29 (t, J=54 Hz, 1H), 4.57 (d, 1H), 4.47 (d, 1H), 4.21 (d, 1H), 4.18 (d, 1H).

### c) 5-Difluoromethyl-5-(2-fluoro-phenyl)-5,6-dihydro-2H-[1,4]oxazin-3-ylamine

5-Difluoromethyl-5-(2-fluoro-phenyl)-morpholine-3-thione (160 g, 570 mmol) was dissolved in 2.4 L of a NH₃ solution 7 mol/L in methanol for 6.5 h and afterwards left standing overnight. The reaction mixture was evaporated and taken up in 2 L 1N aqueous HCl and 2 L TBME. The aqueous phase was washed with TBME and made basic by the addition of 300 ml 30% aqueous NaOH and some ice. The mixture was extracted with DCM three times and the combined organic layers were dried with Na₂SO₄ and concentrated in vacuo. The title compound was obtained by crystallization from DCM/heptanes (128.45 g).

HPLC: Rt_{H3}=2.059 min; ESIMS [M+H]⁺=245;

¹H-NMR (CDCl₃, 360 MHz): 7.77 (t, 1H), 7.38-7.30 (m, 1H), 7.21 (t, 1H), 7.09 (dd, 1H), 6.19 (t, J=54 Hz, 1H), 4.51 (br, 2H), 4.32, (d, 1H), 4.18 (d, 1H), 4.05 (d, 1H), 3.96 (d, 1H), 1.39 (s, 3H), 1.24 (s, 3H).

### d) 5-Difluoromethyl-5-(2-fluoro-5-nitro-phenyl)-5,6-dihydro-2H-[1,4]oxazin-3-ylamine

Potassium nitrate (60.3 g, 596 mmol) was added portionwise to 600 ml sulfuric acid (Temperature <20° C.). This solution was added dropwise to a solution of 5-difluoromethyl-5-(2-fluoro-phenyl)-5,6-dihydro-2H-[1,4]oxazin-3-ylamine (112 g, 459 mmol) in 600 ml sulfuric acid, while keeping the reaction temperature <22° C. with an ice bath. After stirring for 1 h, the mixture was poured onto 10 Kg ice. TBME (6 L) was added and the pH was adjusted to 12-14 by the addition of about 5 L 30% aqueous NaOH. The phases were separated and the aqueous phase was extracted twice with TBME. The compined organic layers were dried with sodium sulfate and evaporated to give 130 g of a yellow solid that was used further without purification.

HPLC: Rt_{H3}=2.063 min; ESIMS [M+H]⁺=290;

¹H-NMR (CDCl₃, 360 MHz): 8.71 (dd, 1H), 8.13 (dt, 1H), 7.13 (dd, 1H), 5.99 (t, J=54 Hz, 1H), 4.55 (br, 2H), 4.33 (dd, 1H), 4.10 (d, 1H), 3.97 (d, 1H), 3.82 (dt, 1H).

### e) [5-Difluoromethyl-5-(2-fluoro-5-nitro-phenyl)-5,6-dihydro-2H-[1,4]oxazin-3-yl]-carbamic acid tert-butyl ester

A solution of 5-Difluoromethyl-5-(2-fluoro-5-nitro-phenyl)-5,6-dihydro-2H-[1,4]oxazin-3-ylamine (144.5 g, 500 mmol), Boc anhydride (142 g, 650 mmol) and DIPEA (131 ml, 749 mmol) in 2500 ml THF was stirred for 3 days at rt, after which there was still starting material remaining. Boc anhydride (56 g, 325 mmol) was added, the mixture heated to 60° C. and stirred for 10 h until the reaction was complete. The mixture was evaporated, dissolved in TBME, washed with ice-cold 1N aqueous HCl, water, 10% aqueous NaHCO₃ and brine. The organic phase was dried with sodium sulfate, filtered and evaporated. The product was purified by crystallization from DCM/heptanes. Yield 182.8 g white crystal's.

HPLC: Rt_{H1}=3.259 min; ESIMS [M+Na]⁺=412;

¹H-NMR (CDCl₃, 360 MHz): 8.70 (dd, 1H), 8.27 (dt, 1H), 7.34 (br, 1H), 7.25 (dd, 1H), 6.09 (t, J=54 Hz, 1H), 4.85 (d, 1H), 4.58 (d, 1H), 4.49 (dd, 1H), 3.94 (dt, 1H).

### f) [5-(5-Amino-2-fluoro-phenyl)-5-difluoromethyl-5,6-dihydro-2H-[1,4]oxazin-3-yl]-carbamic acid tert-butyl ester

[5-Difluoromethyl-5-(2-fluoro-5-nitro-phenyl)-5,6-dihydro-2H-[1,4]oxazin-3-yl]-carbamic acid tert-butyl ester (180 g, 462 mmol) and 17.61 g Pd—C 10% were suspended in 1760 mL THF. The mixture was shaken in a Parr apparatus in an atmosphere of hydrogen at rt. After 6 h the reaction mixture was filtered over celite and evaporated. The residue was crystallized from DCM/heptanes to provide 157.6 g of the title compound as beige crystals.

HPLC: Rt_{H3}=2.748 min; ESIMS [M+H]⁺=360;

¹H-NMR (CDCl₃, 360 MHz): Spectrum uninterpretable due to the presence of a complex mixture of rotamers.

### g) [(R)-5-(5-Amino-2-fluoro-phenyl)-5-difluoromethyl-5,6-dihydro-2H-[1,4]oxazin-3-yl]-carbamic acid tert-butyl ester

The racemic product ((rac)[5-(5-Amino-2-fluoro-phenyl)-5-difluoromethyl-5,6-dihydro-2H-[1,4]oxazin-3-yl]-carbamic acid tert-butyl ester) was separated via prep-HPLC on Chiralpak AD-H 20 um (8×100×48 mm HPLC columns), on a Bayer SMB CC50 instrument using SMB technology with heptane/EtOH/MeOH 70:20:10 as eluent. The desired compound was the slower eluting (R)-enantiomer. Yielding 72.29 g of the title compound as a colourless foam. ee=99.3%; Opt. rotation: [α]_{D} −97.5° (c=1, CHCl₃)

HPLC: Rt_{H3}=2.748 min; ESIMS [M+H]⁺=360;

¹H-NMR (CDCl₃, 360 MHz): Spectrum uninterpretable due to the presence of a complex mixture of rotamers.

### h) ((R)-5-{5-[(5-Cyano-pyridine-2-carbonyl)-amino]-2-fluoro-phenyl}-5-difluoromethyl-5,6-dihydro-2H-[1,4]oxazin-3-yl)-carbamic acid tert-butyl ester

[(R)-5-(5-Amino-2-fluoro-phenyl)-5-difluoromethyl-5,6-dihydro-2H-[1,4]oxazin-3-yl]-carbamic acid tert-butyl ester (35 g, 97.4 mmol), 5-cyano-pyridine-2-carboxylic acid (15.87 g, 107.14 mmol) and HOBt hydrate (22.35 g, 146.1 mmol) were dissolved in 185 ml DMF and stirred with ice cooling. When the temperature had reached 0-5° C. EDC (22.33 ml, 126.62 mmol) was added dropwise. The mixture was stirred for 2 h. The ice bath was taken away and stirring was continued for 2 h. The mixture was taken up in EtOAc and water. The phases were separated and the organic phase was washed with 5% aqueous NaHCO₃ and brine. The organic phase was dried with MgSO₄.H₂O and evaporated to provide a beige solid. Crystallisation from EtOAc/hexane gave the title compound as colorless crystals. Yield 44.47 g.

HPLC: Rt_{H1}=2.888 min; ESIMS [M+Na]⁺=512;

¹H-NMR (CDCl₃, 360 MHz, signals broadened due to rotamers): 8.95 (s, 1H), 8.48 (d, 1H), 8.25 (d, 1H), 8.08-8.03 (m, 1H), 7.84-7.80 (m, 1H), 7.37 (s, 1H), 7.17 (t, 1H), 6.18 (t, J=54 Hz, 1H), 4.83 (d, 1H), 4.60 (d, 1H), 4.42 (d, 1H), 4.4-4.3 (br, 1H), 3.97 (d, 1H), 1.53 (s, 9H).

### i) 5-Cyano-pyridine-2-carboxylic acid [3-((R)-5-amino-3-difluoromethyl-3,6-dihydro-2H-[1,4]oxazin-3-yl)-4-fluoro-phenyl]-amide

((R)-5-{5-[(5-Cyano-pyridine-2-carbonyl)-amino]-2-fluoro-phenyl}-5-difluoromethyl-5,6-dihydro-2H-[1,4]oxazin-3-yl)-carbamic acid tert-butyl ester (44.47 g, 91.0 mmol) was dissolved in 450 ml DCM and mildly chilled with a rt water bath. TFA (150 ml) was added. The reaction was slightly exothermic. The mixture was stirred for 1.5 h at rt. The volatiles were removed with vacuum at rt. The residue was taken up in DCM and the procedure repeated twice. The residue was taken up in 3 L EtOAc and washed with 10% aqueous Na₂CO₃ and brine. The organic phase was dried with sodium sulfate and partially evaporated. iPrOH was added and the mixture chilled. The title compound was collected as snow-white crystals. Yield 30.56 g.

HPLC: Rt_{H3}=2.605 min; ESIMS [M+H]⁺=390;

¹H-NMR (dmso-d6, 600 MHz): 10.85 (s, 1H), 9.22 (s, 1H), 8.58 (d, 1H), 8.27 (d, 1H), 8.18-8.14 (m, 1H), 7.85-7.80 (m, 1H), 7.19 (t, 1H), 6.16 (br s, 2H), 6.14 (t, J=54 Hz, 1H), 4.12 (d, 1H), 4.01 (d, 1H), 3.92 (d, 1H), 3.88 (d, 1H).

### j) 5-Cyano-pyridine-2-carboxylic acid [3-((R)-5-amino-3-difluoro-methyl-3,6-dihydro-2H-[1,4]oxazin-3-yl)-4-fluoro-phenyl]-amide, hydrochloride

A solution of 5-cyano-pyridine-2-carboxylic acid [3-((R)-5-amino-3-difluoro-methyl-3,6-dihydro-2H-[1,4]oxazin-3-yl)-4-fluoro-phenyl]-amide (277 mg, 0.71 mmol) in 5 ml THF was triturated with 0.9 ml of 1M HCl in Et₂O. The mixture was partially evaporated, diluted with TBME and partially evaporated (3×), finally to dryness. The hydrochloride salt contained a significant amount of THF. It was taken up in EtOH and evaporated to dryness twice. The product was finally lyophilized with 15 ml water. Yield 261 mg white lyophilisate.

¹H-NMR (dmso-d6, 600 MHz): 11.05 (s, 1H), 11.01 (s, 1H), 9.75 (s, 1H), 9.25 (s, 1H), 8.73 (br s, 1H), 8.61 (d, 1H), 8.10 (d, 1H), 8.12-8.07 (m, 2H), 7.41 (dd, 1H), 6.79 (t, J=54 Hz, 1H), 4.70 (d, 1H), 4.65 (d, 1H), 4.36 (d, 1H), 4.18 (d, 1H).

### Example 152

### Crystalline 5-cyano-pyridine-2-carboxylic acid [3-((R)-5-amino-3-difluoromethyl-3,6-dihydro-2H-[1,4]oxazin-3-yl)-4-fluoro-phenyl]-amide

5-cyano-pyridine-2-carboxylic acid [3-((R)-5-amino-3-difluoromethyl-3,6-dihydro-2H-[1,4]oxazin-3-yl)-4-fluoro-phenyl]-amide was dissolved in EtOAc, isopropanol added and the resulting solution concentrated at reduced pressure. This procedure was repeated until most of the product had crystallised.

The resultant crystalline material was analysed by XRPD and the ten most characteristic peaks are shown in Table 18 (see also FIG. 1).

TABLE 18

| | | | Relative |
|---|---|---|---|
| Degrees 2-⊖ | d-spacing (Å) | Intensity (counts) | Intensity % |
| 8.29 | 10.65649 | 9840 | High |
| 10.813 | 8.17512 | 5198 | Medium |
| 14.077 | 6.28645 | 1911 | Low |
| 14.525 | 6.09337 | 2446 | Low |
| 16.624 | 5.32842 | 19854 | High |
| 18.919 | 4.68693 | 3766 | Medium |
| 21.453 | 4.13863 | 3862 | Medium |
| 22.244 | 3.99323 | 6947 | Medium |
| 23.327 | 3.81033 | 4257 | Medium |
| 25.436 | 3.49889 | 3672 | Medium |
| 28.495 | 3.12985 | 5558 | Medium |

X-ray powder diffraction (XRPD) analysis was performed using a Brucker D8 Advance x-ray diffractometer. Measurements were taken at about 30 kV and 40 mA under the following conditions:
Scan rate (continuous scan): 0.3 s/step (equals 107.1 s step time)
Step size: 0.017° (2Theta)
Soller slit 2.5°
Slits (from left to right): V12 (variable), 6 mm antiscatter slit

The X-ray diffraction pattern was recorded between 2° and 40° (2 theta) with CuK_{α} radiation for identification of the whole pattern.

The crystalline material was also analysed by differential scanning calorimetry using a Perkin Elmer DSC7 and was found to have an onset of melting at about 227° C. (227.46° C.).

### Example 153

The compound in Table 19 can be prepared by procedures analogous to those used in Examples 71 and 72.

TABLE 19

| | | ¹H-NMR | MS |
|---|---|---|---|
| Example | Compound | (δ; DMSO-d₆) | [m/z; (M + 1)⁺] |
| 153 | 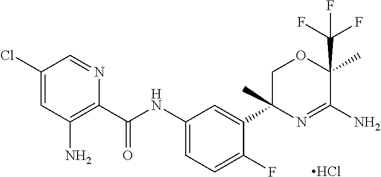 [SMILES: C[C@@]1(c2cc(NC(=O)c3ncc(Cl)cc3N)ccc2F)CO[C@@](C)(C(F)(F)F)C(N)=N1 ; Cl] 3-Amino-5-chloro-pyridine-2-carboxylic acid [3-((3R,6R)-5-amino-3,6-dimethyl-6- trifluoromethyl-3,6-dihydro-2H-[1,4]oxazin- 3-yl)-4-fluoro-phenyl]-amide hydrochloride | 11.84 (s, 1H), 10.71 (s, 1H), 9.71 (d, 2H), 7.99 (m, 1H), 7.87 (m, 2H), 7.38 (s, 1H), 7.29 (dd, 1H), 7.19 (br s, 2H), 4.31 (d, 1H), 4.06 (d, 1H), 1.75 (s, 3H), 1.71 (s, 3H) | 460, 462 |

### Examples 154 to 156

The compounds listed in Table 20 were prepared by a procedure analogous to that used in Example 100.

TABLE 20

| | | ¹H-NMR | MS |
|---|---|---|---|
| Example | Compound | (δ; DMSO-d₆) | [m/z; (M + 1)⁺] |
| 154 | 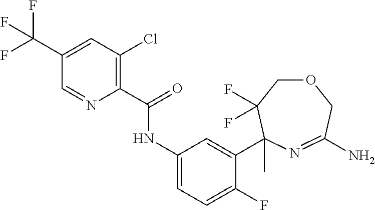 [SMILES: CC1(c2cc(NC(=O)c3ncc(C(F)(F)F)cc3Cl)ccc2F)N=C(N)COCC1(F)F] 3-Chloro-5-trifluoromethyl- pyridine-2- carboxylic acid [3-(3-amino-6,6-difluoro- 5-methyl-2,5,6,7-tetrahydro-[1,4]oxazepin-5-yl)- 4-fluoro-phenyl]-amide | 11.93 (s, 1H), 9.08 (s, 1H), 8.72 (s, 1H), 7.85 (m, 2H), 7.23 (s, 1H), 4.68-3.93 (m, 4H), 1.82 (s, 3H) | 481, 483 |
| 155 | 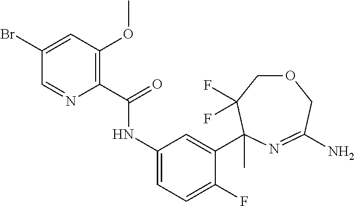 [SMILES: COc1cc(Br)cnc1C(=O)Nc1ccc(F)c(C2(C)N=C(N)COCC2(F)F)c1] 5-Bromo-3-methoxy-pyridine-2- carboxylic acid [3-(3-amino-6,6 difluoro- 5-methyl-2,5,6,7 tetrahydro- [1,4]oxazepin-5-yl)-4 fluoro-phenyl]-amide | 10.49 (s, 1H), 8.33 (s, 1H), 7.95 (s, 1H), 7.86-7.87 (m, 2H), 7.08 (dd, 1H, J = 11.0 Hz, 8.4 Hz), 5.98 (s, 1H), 4.32-4.05 (m, 3H), 3.96-3.81 (m, 4H), 3.53-3.45 (m, 1H), 1.74 (s, 3H) | 487 |
| 156 | 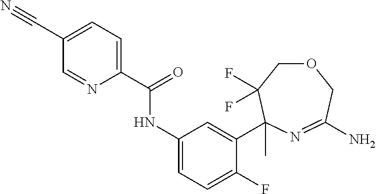 [SMILES: CC1(c2cc(NC(=O)c3ccc(C#N)cn3)ccc2F)N=C(N)COCC1(F)F] 5-Cyano-pyridine-2-carboxylic acid [3- (3-amino-6,6-difluoro-5 methyl-2,5,6,7- tetrahydro[1,4]oxazepin-5-yl)-4 fluoro- phenyl]-amide | 10.8 (s, 1H), 9.19 (d, 1H, J = 3.5 Hz), 8.58 (dd, 1H, J = 10.3 Hz, 3.2 Hz), 8.27 (dd, 1H, J = 10.5 Hz, 2.7 Hz), 8.07 (dd, 1H, J = 9.5 Hz, 3.6 Hz), 7.85 (m, 1H), 7.12 (dd, 1H, J = 14 Hz, 10.5 Hz), 7.95 (s, 2H), 4.31-4.08 (m, 3H), 3.96-3.84 (m, 1H), 1.75 (s, 3H) | 404 |

(Note: For example 156 the deprotection of the Boc group was carried out using TFA/DCM (in an analogous manner as for example 112) instead of HCl/dioxane.

### Examples 157 to 185

The compounds listed in Table 21 were prepared by procedures analogous to those used in Example 42 or Example 112.

For enantiomerically pure compounds the racemic precursor [5-(5-amino-2-fluoro-phenyl)-5-difluoromethyl-5,6-dihydro-2H-[1,4]oxazin-3-yl]-carbamic acid tert-butyl ester (example 42j)) was separated via prep-HPLC on Chiralpak AD-H 250×4.6 mm column using supercritical CO2/EtOH 9:1 as an eluent. The desired compound was the slower eluting (R)-enantiomer. Enantiomeric excess=99.7%; [α]_{D}=−109.7° (c=1, CHCl₃).

TABLE 21

| | | ¹H-NMR | MS |
|---|---|---|---|
| Example | Compound | (δ; DMSO-d₆) | [m/z; (M + 1)⁺] |
| 157 | 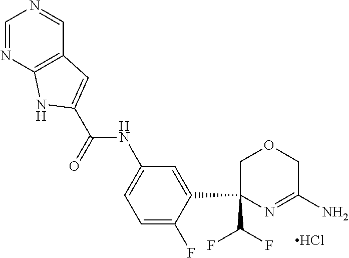 [SMILES: NC1=N[C@@](c2cc(NC(=O)c3cc4cncnc4[nH]3)ccc2F)(C(F)F)COC1 ; Cl] 7H-Pyrrolo[2,3-d]pyrimidine-6-carboxylic acid [3-((R)-5-amino-3-difluoromethyl-3,6- dihydro-2H-[1,4]oxazin-3-yl)-4-fluoro- phenyl]-amide hydrochloride | 13.85 (s, 1H), 11.2 (s, 1H), 11.15 (s, 1H), 9.88 (s, 1H), 9.55 (s, 1H), 9.25 (s, 1H), 8.91 (s, 1H), 8.1 (m, 1H), 8.00 (m, 1H), 7.95 (s, 1H), 7.45-7.40 (dd, 1H), 6.80 (t, 1H, CHF₂), 4.72 (d, 1H, AB-system), 4.67 (d, 1H, AB-system), 4.35 (d, 1H, AB- system), 4.20 (d, 1H, AB-system) | 405 |
| 158 | 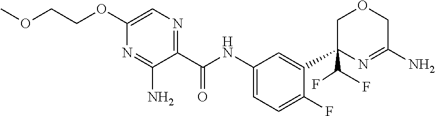 [SMILES: COCCOc1cnc(C(=O)Nc2ccc(F)c([C@]3(C(F)F)COCC(N)=N3)c2)c(N)n1] 3-Amino-5-(2-methoxy-ethoxy)-pyrazine-2- carboxylic acid [3-((R)-5-amino-3- difluoromethyl-3,6-dihydro-2H-[1,4]oxazin-3- yl)-4-fluoro-phenyl]-amide | 10.14 (br s, 1H), 8.03 (d, 1H), 7.75 (br s, 1H), 7.53 (s, 1H), 7.14 (t, 1H), 6.16 (br s, 2H), 4.41 (br. s., 2H), 4.14 (d, 1H), 4.01 (d, 1H), 3.91 (d, 1H), 3.81 (d, 1H), 3.72-3.66 (m, 3H), 3.50 (s, 1H), 3.30 (s, 3H) | 455 |
| 159 | 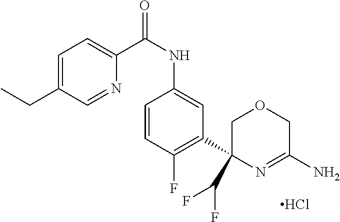 [SMILES: CCc1ccc(C(=O)Nc2ccc(F)c([C@]3(C(F)F)COCC(N)=N3)c2)nc1 ; Cl] ((R)-5-Difluoromethyl-5-{5-[(5-ethyl-pyridine- 2-carbonyl)-amino]-2-fluoro-phenyl}-5,6- dihydro-2H-[1,4]oxazin-3-yl)-carbamic acid tert-butyl ester, hydrochloride | 10.85 (s, 1H), 9.80 (s, 1H), 8.84 (s, 1H), 8.64 (s, 1H), 8.13-8.08 (m, 3H), 7.94 (d, 1H), 7.38 (dd, 1H), 6.78 (t, J = 54 Hz, 1H), 4.73 (d, 1H), 4.61 (d, 1H), 4.36 (d, 1H), 4.18 (d, 1H), 2.78 (q, 2H), 1.24 (t, 3H) | 393 |
| 160 | 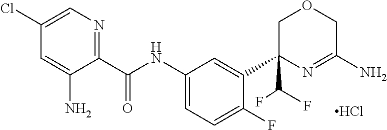 [SMILES: NC1=N[C@@](c2cc(NC(=O)c3ncc(Cl)cc3N)ccc2F)(C(F)F)COC1 ; Cl] 3-Amino-5-chloro-pyridine-2-carboxylic acid [3-((R)-5-amino-3-difluoromethyl-3,6- dihydro-2H-[1,4]oxazin-3-yl)-4-fluoro- phenyl]-amide hydrochloride | 10.99 (s, 1H), 10.60 (s, 1H), 9.78 (br s, 1H), 8.80 (br s, 1H), 8.05 (d, 1H), 7.93 (d, 1H), 7.86 (s, 1H), 7.42-7.30 (m, 2H), 7.17 (br s, 2H), 6.77 (t, 1H, CHF2), 4.78-4.67 (m, 1H), 4.67-4.57 (m, 1H), 4.34 (d, 1H), 4.16 (d, 1H) | 414, 416 |
| 161 | 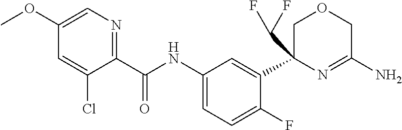 [SMILES: COc1cnc(C(=O)Nc2ccc(F)c([C@]3(C(F)F)COCC(N)=N3)c2)c(Cl)c1] 3-Chloro-5-methoxy-pyridine-2-carboxylic acid [3-((R)-5-amino-3-difluoromethyl-3,6- dihydro-2H-[1,4]oxazin-3-yl)-4-fluoro- phenyl]-amide | 10.6 (s, 1H), 8.33 (s, 1H), 7.97 (m, 1H), 7.78 (m, 1H), 7.72 (s, 1H), 7.17 (dd, 1H), 6.14 (t, 1H), 6.13 (br s, 2H), 4.15 (d, 1H), 4.02 (d, 1H), 3.93 (s, 3H), 3.91 (d, 1H), 3.83 (d, 1H) | 429, 431 |
| 162 | 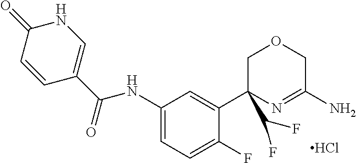 [SMILES: NC1=N[C@@](c2cc(NC(=O)c3ccc(=O)[nH]c3)ccc2F)(C(F)F)COC1 ; Cl] 6-Oxo-1,6-dihydro-pyridine-3-carboxylic acid [3-((R)-5-amino-3-difluoromethyl-3,6- dihydro-2H-[1,4]oxazin-3-yl)-4-fluoro- phenyl]-amide hydrochloride | 12.20 (s, 1H), 11.02 (s, 1H), 10.32 (s, 1H), 9.78 (s, 1H), 8.79 (s, 1H), 8.24 (s, 1H), 7.97 (dd, 1H), 7.92 (dd, 1H), 7.79 (m, 1H), 7.35 (t, 1H), 6.77 (t, 1H, CHF₂), 6.41 (d, 1H), 4.72 (d, 1H, AB-system), 4.65 (d, 1H, AB-system), 4.32 (d, 1H, AB-system), 4.18 (d, 1H, AB-system) | 381 |
| 163 | 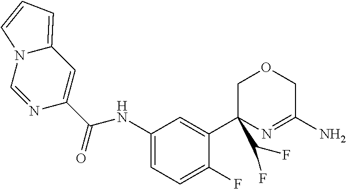 [SMILES: NC1=N[C@@](c2cc(NC(=O)c3cc4cccn4cn3)ccc2F)(C(F)F)COC1] Pyrrolo[1,2-c]pyrimidine-3-carboxylic acid [3-((R)-5-amino-3-difluoromethyl-3,6- dihydro-2H-[1,4]oxazin-3-yl)-4-fluoro- phenyl]-amide | 10.30 (s, 1H), 9.30 (s, 1H), 8.21 (s, 1H), 8.09 (m, 1H), 7.90 (s, 1H), 7.87 (m, 1H), 7.18 (dd, 1H), 7.05 (t, 1H), 6.83 (d, 1H), 6.18 (s, NH2), 6.14 (t, 1H, CHF2), 4.12 (d, 1H, AB- system), 4.02 (d, 1H, AB-system), 3.93 (d, 1H, AB-system), 3.82 (d, 1H, AB-system) | 404 |
| 164 | 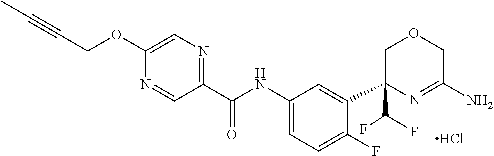 [SMILES: CC#CCOc1cnc(C(=O)Nc2ccc(F)c([C@]3(C(F)F)COCC(N)=N3)c2)cn1 ; Cl] 5-But-2-ynyloxy-pyrazine-2-carboxylic acid [3-((R)-5-amino-3-difluoromethyl-3,6- dihydro-2H-[1,4]oxazin-3-yl)-4-fluoro- phenyl]-amide hydrochloride | 11.02 (s, 1H), 10.78 (s, 1H), 9.66 (s, 1H), 8.91 (s, 1H), 8.78 (s, 1H), 8.46 (s, 1H), 8.11 (d, 1H), 8.03 (dd, 1H), 7.37 (dd, 1H), 6.77 (t, 1H, CHF2), 5.08 (d, 2H), 4.76-4.67 (m, 1H), 4.67-4.58 (m, 1H), 4.33 (d, 1H), 4.17 (d, 1H), 1.84 (s, 3H) | 434 |
| 165 | 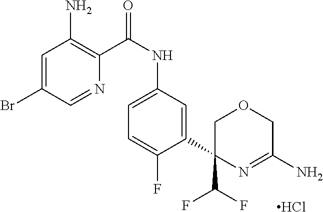 [SMILES: NC1=N[C@@](c2cc(NC(=O)c3ncc(Br)cc3N)ccc2F)(C(F)F)COC1 ; Cl] 3-Amino-5-bromo-pyridine-2-carboxylic acid [3-((R)-5-amino-3-difluoromethyl-3,6- dihydro-2H-[1,4]oxazin-3-yl)-4-fluoro- phenyl]-amide hydrochloride | 10.98 (s, 1H), 10.13 (s, 1H), 9.74 (s, 1H), 8.72 (s, 1H), 8.04 (d, 1H), 7.95-7.91 (m, 2H), 7.52 (s, 1H), 7.36 (dd, 1H), 7.12 (br, 2H), 6.78 (t, J= 54 Hz, 1H), 4.72 (d, 1H), 4.64 (d, 1H), 4.32 (d, 1H), 4.15 (d, 1H) | 458, 460 |
| 166 | 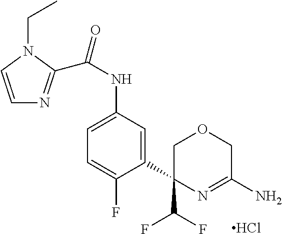 [SMILES: CCn1ccnc1C(=O)Nc1ccc(F)c([C@]2(C(F)F)COCC(N)=N2)c1 ; Cl] 1-Ethyl-1H-imidazole-2-carboxylic acid [3- ((R)-5-amino-3-difluoromethyl-3,6-dihydro- 2H-[1,4]oxazin-3-yl)-4-fluoro-phenyl]-amide hydrochloride | 11.15 (s, 1H), 11.07 (s, 1H), 9.98 (s, 1H), 9.09 (s, 1H), 7.96 (m, 2H), 7.73 (s, 1H), 7.37 (m, 2H), 6.74 (t, 1H, CHF2), 4.71 (d, 1H), 4.63 (d, 1H), 4.47 (q, 2H), 4.31 (d, 1H), 4.14 (d, 1H), 1.37 (t, 2H) | 382 |
| 167 | 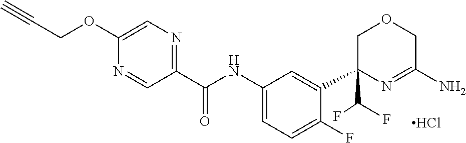 [SMILES: C#CCOc1cnc(C(=O)Nc2ccc(F)c([C@]3(C(F)F)COCC(N)=N3)c2)cn1 ; Cl] 5-Prop-2-ynyloxy-pyrazine-2-carboxylic acid [3-((R)-5-amino-3-difluoromethyl-3,6- dihydro-2H-[1,4]oxazin-3-yl)-4-fluoro- phenyl]-amide hydrochloride | 10.54 (br s, 1H), 8.90 (s, 1H), 8.49 (s, 1H), 8.14 (dd, 1H), 7.81 (br s, 1H), 7.19 (t, 1H), 6.24-6.06 (m, 3H), 5.14 (d, 2H), 4.12 (d, 1H), 4.03-4.01 (m, 1H), 3.94-3.91 (m, 1H), 3.84 (d, 1H), 3.66 (s, 1H) | 420 |
| 168 | 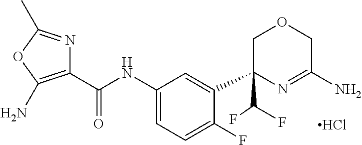 [SMILES: Cc1nc(C(=O)Nc2ccc(F)c([C@]3(C(F)F)COCC(N)=N3)c2)c(N)o1 ; Cl] 5-Amino-2-methyl-oxazole-4-carboxylic acid [3-((R)-5-amino-3-difluoromethyl-3,6- dihydro-2H-[1,4]oxazin-3-yl)-4-fluoro- phenyl]-amide hydrochloride | 10.96 (s, 1H), 9.79 (s, 1H), 9.62 (s, 1H), 8.80 (s, 1H), 8.00-7.96 (m, 1H), 7.92 (d, 1H), 7.33- 7.20 (m, 1H), 7.00 (br s, 1H), 6.76 (t, 1H, CHF2), 4.76-4.56 (m, 2H), 4.32 (d, 1H), 4.14 (d, 1H), 2.31 (s, 3H) | 384 |
| 169 | 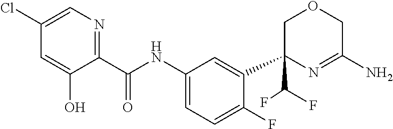 [SMILES: NC1=N[C@@](c2cc(NC(=O)c3ncc(Cl)cc3O)ccc2F)(C(F)F)COC1] 5-Chloro-3-hydroxy-pyridine-2-carboxylic acid [3-((R)-5-amino-3-difluoromethyl-3,6- dihydro-2H-[1,4]oxazin-3-yl)-4-fluoro- phenyl]-amide | 11.1 (d, 1H), 9.68 (s, 1H), 8.84 (s, 1H), 8.28 (s, 1H), 8.0 (m, 2H), 7.73 (s, 1H), 7.40 (dd, 1H), 6.77 (t, 1H), 4.67 (d, 1H), 4.63 (d, 1H), 4.34 (d, 1H), 4.17 (d, 1H) | 415, 417 |
| 170 | 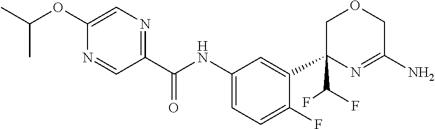 [SMILES: CC(C)Oc1cnc(C(=O)Nc2ccc(F)c([C@]3(C(F)F)COCC(N)=N3)c2)cn1] 5-Isopropoxy-pyrazine-2-carboxylic acid [3- ((R)-5-amino-3-difluoromethyl-3,6-dihydro- 2H-[1,4]oxazin-3-yl)-4-fluoro-phenyl]-amide | 10.49 (br s, 1H), 8.85 (s, 1H), 8.33 (s, 1H), 8.13 (d, 1H), 7.80 (br s, 1H), 7.19 (dd, 1H), 6.15 (br s, 2H), 6.13 (t, 1H), 5.34 (m, 1H), 4.16 (d, 1H), 4.02 (d, 1H), 3.87 (d, 1H), 3.71 (d, 1H), 1.35 (d, 6H) | 424 |
| 171 | 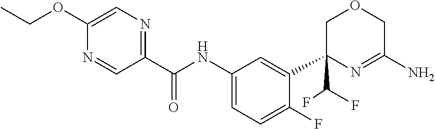 [SMILES: CCOc1cnc(C(=O)Nc2ccc(F)c([C@]3(C(F)F)COCC(N)=N3)c2)cn1] 5-Ethoxy-pyrazine-2-carboxylic acid [3-((R)-5-amino-3-difluoromethyl-3,6- dihydro-2H-[1,4]oxazin-3-yl)-4-fluoro- phenyl]-amide | 10.49 (br s, 1H), 8.87 (s, 1H), 8.37 (s, 1H), 8.17 (d, 1H), 7.81 (br s, 1H), 7.18 (dd, 1H), 6.16 (br s, 2H), 6.14 (t, 1H), 4.43 (t, 2H), 3.7-4.2 (m, 4H), 1.35 (t, 3H) | 410 |
| 172 | 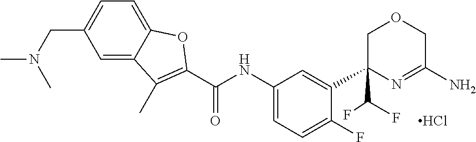 [SMILES: Cc1c(C(=O)Nc2ccc(F)c([C@]3(C(F)F)COCC(N)=N3)c2)oc2ccc(CN(C)C)cc12 ; Cl] 5-Dimethylaminomethyl-3-methyl- benzofuran-2-carboxylic acid [3-((R)-5- amino-3-difluoromethyl-3,6-dihydro-2H- [1,4]oxazin-3-yl)-4-fluoro-phenyl]-amide hydrochloride | 11.07 (s, 1H), 10.80 (br s, 1H), 10.75 (s, 1H), 9.84 (s, 1H), 8.88 (s, 1H), 8.09-7.94 (m, 3H), 7.84-7.70 (m, 2H), 7.40 (dd, 1H), 6.79 (t, 1H, CHF2), 4.80-4.70 (m, 2H), 4.41-4.30 (m, 3H), 4.19 (d, 1H), 2.72 (s, 3H), 2.71 (s, 3H), 2.61 (s, 3H) | 475 |
| 173 | 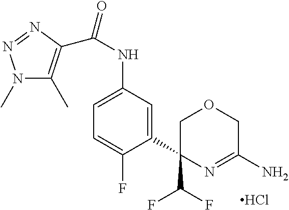 [SMILES: Cc1c(C(=O)Nc2ccc(F)c([C@]3(C(F)F)COCC(N)=N3)c2)nnn1C ; Cl] 1,5-Dimethyl-1H-[1,2,3]triazole-4-carboxylic acid [3-((R)-5-amino-3-difluoromethyl-3,6- dihydro-2H-[1,4]oxazin-3-yl)-4-fluoro- phenyl]-amide hydrochloride | 11.04 (s, 1H), 10.57 (s, 1H), 9.87 (s, 1H), 8.93 (s, 1H), 7.99 (m, 2H), 7.31 (m, 2H), 6.74 (t, 1H, CHF2), 4.70 (d, 1H), 4.62 (d, 1H), 4.31 (d, 1H), 4.14 (d, 1H), 3.98 (s, 3H), 2.54 (s, 3H) | 383 |
| 174 | 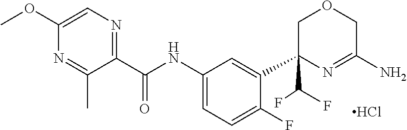 [SMILES: COc1cnc(C(=O)Nc2ccc(F)c([C@]3(C(F)F)COCC(N)=N3)c2)c(C)n1 ; Cl] 5-Methoxy-3-methyl-pyrazine-2-carboxylic acid [3-((R)-5-amino-3-difluoromethyl-3,6- dihydro-2H-[1,4]oxazin-3-yl)-4-fluoro- phenyl]-amide hydrochloride | 10.99 (s, 1H), 10.68 (s, 1H), 9.75 (s, 1H), 8.74 (s, 1H), 8.26 (s, 1H), 8.06-7.92 (m, 2H), 7.44-7.32 (m, 1H), 6.78 (t, 1H, CHF2), 4.78-4.68 (m, 2H), 4.34 (d, 1H), 4.18 (d, 1H), 4.00 (s, 3H), 2.77 (s, 3H) | 410 |
| 175 | 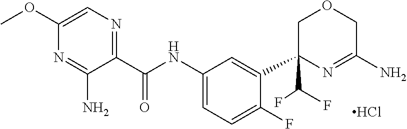 [SMILES: COc1cnc(C(=O)Nc2ccc(F)c([C@]3(C(F)F)COCC(N)=N3)c2)c(N)n1 ; Cl] 3-Amino-5-methoxy-pyrazine-2-carboxylic acid [3-((R)-5-amino-3-difluoromethyl-3,6- dihydro-2H-[1,4]oxazin-3-yl)-4-fluoro- phenyl]-amide hydrochloride | 10.98 (s, 1H), 10.41 (s, 1H), 9.76 (s, 1H), 8.77 (s, 1H), 8.14-7.98 (m, 1H), 7.98-7.82 (m, 1H), 7.54 (s, 1H), 7.34 (dd, 1H), 4.80-4.67 (m, 1H), 4.67-4.53 (m, 1H), 4.34 (d, 1H), 4.16 (d, 1H), 3.91 (s, 3H) | 411 |
| 176 | 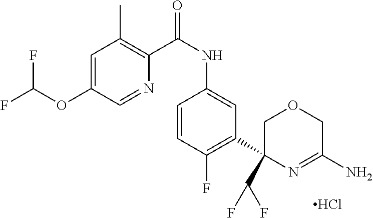 [SMILES: Cc1cc(OC(F)F)cnc1C(=O)Nc1ccc(F)c([C@]2(C(F)F)COCC(N)=N2)c1 ; Cl] 5-Difluoromethoxy-3-methyl-pyridine-2- carboxylic acid [3-((R)-5-amino-3- difluoromethyl-3,6-dihydro-2H-[1,4]oxazin-3- yl)-4-fluoro-phenyl]-amide hydrochloride | 10.91 (br s, 1H), 10.75 (s, 1H), 9.66 (br s, 1H), 8.59 (br s, 1H), 8.44 (d, 1H), 8.00-7.98 (m, 2H), 7.75 (d, 1H), 7.45 (t, 1H), 7.41-7.35 (m, 1H), 6.79 (t, 1H), 4.71 (d, 1H), 4.64 (d, 1H), 4.34 (d, 1H), 4.18 (d, 1H), 2.61 (s, 3H) | 445 |
| 177 | 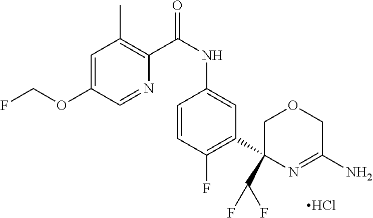 [SMILES: Cc1cc(OCF)cnc1C(=O)Nc1ccc(F)c([C@]2(C(F)F)COCC(N)=N2)c1 ; Cl] 5-Fluoromethoxy-3-methyl-pyridine-2- carboxylic acid [3-((R)-5-amino-3- difluoromethyl-3,6-dihydro-2H-[1,4]oxazin-3- yl)-4-fluoro-phenyl]-amide hydrochloride | 10.92 (br s, 1H), 10.70 (s, 1H), 9.67 (br s, 1H), 8.61 (br s, 1H), 8.38 (d, 1H), 8.00-7.98 (m, 2H), 7.65 (d, 1H), 7.41-7.35 (m, 1H), 6.79 (t, 1H), 6.03 (d, 2H), 4.71 (d, 1H), 4.65 (d, 1H), 4.35 (d, 1H), 4.18 (d, 1H), 2.63 (s, 3H) | 427 |
| 178 | 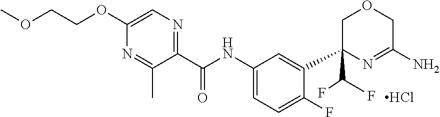 [SMILES: COCCOc1cnc(C(=O)Nc2ccc(F)c([C@]3(C(F)F)COCC(N)=N3)c2)c(C)n1 ; Cl] 5-(2-Methoxy-ethoxy)-3-methyl-pyrazine-2- carboxylic acid [3-((R)-5-amino-3- difluoromethyl-3,6-dihydro-2H-[1,4]oxazin-3- yl)-4-fluoro-phenyl]-amide hydrochloride | 10.98 (s, 1H), 10.68 (s, 1H), 9.74 (s, 1H), 8.72 (br s, 1H), 8.28 (s, 1H), 8.06-7.91 (m, 2H), 7.37 (dd, 1H), 6.78 (t, 1H, CHF2), 4.72-4.63 (m, 2H), 4.57-4.48 (m, 2H), 4.34 (d, 1H), 4.18 (d, 1H), 3.79- 3.63 (m, 2H), 3.31 (s, 3H), 2.76 (s, 3H) | 454 |
| 179 | 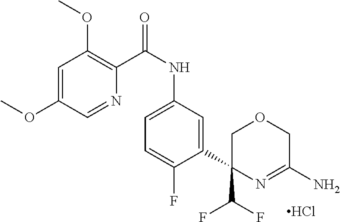 [SMILES: COc1cnc(C(=O)Nc2ccc(F)c([C@]3(C(F)F)COCC(N)=N3)c2)c(OC)c1 ; Cl] 3,5-Dimethoxy-pyridine-2-carboxylic acid [3- ((R)-5-amino-3-difluoromethyl-3,6-dihydro- 2H-[1,4]oxazin-3-yl)-4-fluoro-phenyl]-amide hydrochloride | 11.00 (s, 1H), 10.48 (s, 1H), 9.77 (s, 1H), 8.75 (s, 1H), 8.01 (d, 1H), 7.97 (s, 1H), 7.91-7.87 (m, 1H), 7.34 (t, 1H), 7.22 (br, 2H), 6.78 (t, J = 54 Hz, 1H), 4.69 (d, 1H), 4.65 (d, 1H), 4.33 (d, 1H), 3.94 (s, 3H), 3.89 (s, 3H) | 425 |
| 180 | 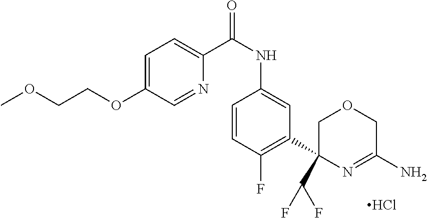 [SMILES: COCCOc1ccc(C(=O)Nc2ccc(F)c([C@]3(C(F)F)COCC(N)=N3)c2)nc1 ; Cl] 5-(2-Methoxy-ethoxy)-pyridine-2-carboxylic acid [3-((R)-5-amino-3-difluoromethyl-3,6- dihydro-2H-[1,4]oxazin-3-yl)-4-fluoro- phenyl]-amide hydrochloride | 10.98 (br s, 1H), 10.74 (s, 1H), 9.76 (br s, 1H), 8.74 (br s, 1H), 8.42 (d, 1H), 8.13 (d, 1H), 8.10- 8.05 (m, 2H), 7.65 (dd, 1H), 7.37 (dd, 1H), 6.79 (t, 1H), 4.72 (d, 1H), 4.64 (d, 1H), 4.34 (d, 1H), 4.31-4.28 (m, 2H), 4.17 (d, 1H), 3.73-3.69 (m, 2H), 3.32 (s, 3H) | 439 |
| 181 | 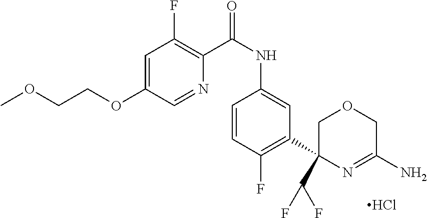 [SMILES: COCCOc1cnc(C(=O)Nc2ccc(F)c([C@]3(C(F)F)COCC(N)=N3)c2)c(F)c1 ; Cl] 3-Fluoro-5-(2-methoxy-ethoxy)-pyridine-2- carboxylic acid [3-((R)-5-amino-3- difluoromethyl-3,6-dihydro-2H-[1,4]oxazin-3- yl)-4-fluoro-phenyl]-amide hydrochloride | 10.99 (br s, 1H), 10.67 (s, 1H), 9.75 (br s, 1H), 8.72 (br s, 1H), 8.31 (d, 1H), 8.03 (d, 1H), 8.00- 7.94 (m, 1H), 7.66 (dd, 1H), 7.37 (dd, 1H), 6.79 (t, 1H), 4.71 (d, 1H), 4.64 (d, 1H), 4.37-4.29 (m, 3H), 4.18 (d, 1H), 3.72-3.69 (m, 2H), 3.32 (s, 3H) | 457 |
| 182 | 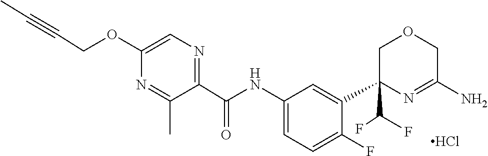 [SMILES: CC#CCOc1cnc(C(=O)Nc2ccc(F)c([C@]3(C(F)F)COCC(N)=N3)c2)c(C)n1 ; Cl] 5-But-2-ynyloxy-3-methyl-pyrazine-2- carboxylic acid [3-((R)-5-amino-3- difluoromethyl-3,6-dihydro-2H-[1,4]oxazin-3- yl)-4-fluoro-phenyl]-amide hydrochloride | 10.94 (s, 1H), 10.70 (s, 1H), 9.69 (s, 1H), 8.64 (s, 1H), 8.30 (s, 1H), 7.98 (d, 2H), 7.38 (t, 1H), 6.79 (t, 1H, CHF2), 5.08 (br s, 2H), 4.75- 4.59 (m, 2H), 4.34 (d, 1H), 4.18 (d, 1H), 2.76 (s, 3H), 1.86 (s, 3H) | 448 |
| 183 | 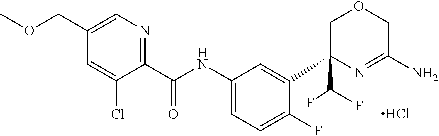 [SMILES: COCc1cnc(C(=O)Nc2ccc(F)c([C@]3(C(F)F)COCC(N)=N3)c2)c(Cl)c1 ; Cl] 3-Chloro-5-methoxymethyl-pyridine-2- carboxylic acid [3-((R)-5-amino-3-di- fluoromethyl-3,6-dihydro-2H-[1,4]oxazin-3- yl)-4-fluoro-phenyl]-amide hydrochloride | 11.21 (s, 1H), 10.96 (s, 1H), 9.95 (s, 1H), 9.09 (s, 1H), 8.59 (s, 1H), 8.04 (s, 1H), 7.94 (m, 1H), 7.90 (d, 1H), 7.40 (dd, 1H), 6.79 (t, 1H), 4.71 (m, 2H), 4.55 (s, 2H), 4.33 (d, 1H), 4.44 (d, 1H), 3.37 (s, 3H) | 443, 445 |
| 184 | 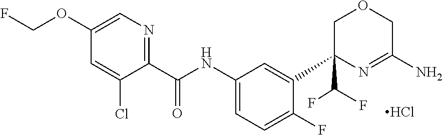 [SMILES: NC1=N[C@@](c2cc(NC(=O)c3ncc(OCF)cc3Cl)ccc2F)(C(F)F)COC1 ; Cl] 3-Chloro-5-fluoromethoxy-pyridine-2- carboxylic acid [3-((R)-5-amino-3-di- fluoromethyl-3,6-dihydro-2H-[1,4]oxazin-3- yl)-4-fluoro-phenyl]-amide hydrochloride | 11.06 (s, 1H). 10.89 (s, 1H), 9.78 (s, 1H), 8.79 (s, 1H), 8.52 (s, 1H), 7.99 (s, 1H), 7.95 (d, 1H), 7.91 (m, 1H), 7.40 (dd, 1H), 6.79 (t, 1H), 6.08 (d, 2H), 4.71 (m, 2H), 4.34 (d, 1H), 4.23 (d, 1H) | 447, 449 |
| 185 | 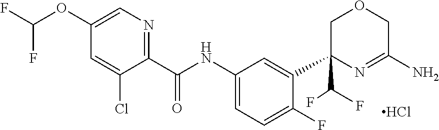 [SMILES: NC1=N[C@@](c2cc(NC(=O)c3ncc(OC(F)F)cc3Cl)ccc2F)(C(F)F)COC1 ; Cl] 3-Chloro-5-difluoromethoxy-pyridine-2- carboxylic acid [3-((R)-5-amino-3- difluoromethyl-3,6-dihydro-2H-[1,4]oxazin-3- yl)-4-fluoro-phenyl]-amide hydrochloride | 11.07 (s, 1H). 10.93 (s, 1H), 9.76 (s, 1H), 8.82 (s, 1H), 8.60 (s, 1H), 8.16 (s, 1H), 7.91 (m, 2H), 7.51 (t, 1H), 7.40 (m, 1H), 6.79 (t, 1H), 4.73 (m, 2H), 4.33 (d, 1H), 4.23 (d, 1H) | 465, 467 |

### Example 186

### 5-Bromo-pyridine-2-carboxylic acid [3-(6-amino-2,4-dimethyl-3-oxo-2,3,4,5-tetrahydro-pyrazin-2-yl)-4-fluoro-phenyl]-amide

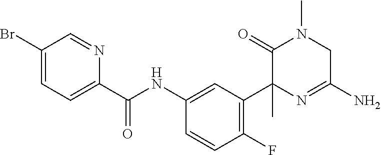 [SMILES: CN1CC(N)=NC(C)(c2cc(NC(=O)c3ccc(Br)cn3)ccc2F)C1=O]

### 186a) 2-Amino-2-(5-bromo-2-fluoro-phenyl)-propionic acid

10 g (50 mmol) of 1-(5-bromo-2-fluoro-phenyl)-ethanone, 6.4 g (100 mmol) of NH₄Cl and 6.5 g (100 mmol) of KCN were dissolved in 200 ml of aq NH₃. The mixture was stirred at rt overnight and extracted with Et₂O, the organic phase was washed with water and brine, dried over Na₂SO₄ and filtered, the filtrate was concentrated in vacuo and taken up in 100 ml of conc hydrochloric acid, and the mixture was refluxed overnight and concentrated in vacuo. The residue was washed twice with DIPE to give the hydrochloride salt of the title compound in the form of a beige powder {HPLC: Rt_{H1}=2.137 min; ESIMS: 262, 264 [(M+H)⁺, 1Br]; ¹H-NMR (360 MHz, D₂O): 7.63 (dd, 1H), 7.51 (ddd, 1H), 7.01 (dd, 1H), 3.56 (s, 3H)}. An aq solution of the salt was treated with 2.2 eq of 2N aq NaOH, washed with TBME and neutralized with 1.2 eq of 2N HCl. The title compound crystallized from the aq solution in the form of colourless crystals {¹H-NMR (400 MHz, CD₃OD): 7.72 (dd, 1H), 7.57 (ddd, 1H), 7.13 (dd, 1H), 1.87 (s, 3H)}.

### 186b) 2-Amino-2-(5-bromo-2-fluoro-phenyl)-propionic acid methyl ester

MeOH (530 ml) was cooled to −10° C. and treated dropwise with 134 ml (1.84 mmol) of SOCl₂. Compound 186a) (50 g, 167.5 mmol) was added in portions. The mixture was slowly heated, stirred at reflux for 18 h, concentrated, taken up in water, washed with TBME, basified with K₂CO₃ and extracted three times with DCM. The combined organic layers were dried over K₂CO₃ and evaporated to yield the title compound in the form of a resin {HPLC: Rt_{H2}=2.266 min; ESIMS: 276, 278 [(M+H)⁺, 1Br]; ¹H-NMR (360 MHz, DMSO-d₆): 7.91 (dd, 1H), 7.54 (ddd, 1H), 7.17 (dd, 1H), 3.62 (s, 3H), 1.48 (s, 3H)}.

### 186c) 2-(5-Bromo-2-fluoro-phenyl)-2-(2-chloro-acetylamino)-propionic acid methyl ester

To a solution of compound 186b) (3.25 g, 11.77 mmol) in 30 ml of DCM were added at −5° C. 2.67 ml (15.30 mmol) of DIPEA and then dropwise 1.037 ml (12.95 mmol) of chloroacetyl chloride. The mixture was stirred for 30 min at −5° C. and then for 1 h without cooling and diluted with TBME and water. The organic phase was washed with water, 1N HCl and brine, dried over MgSO₄×H₂O and evaporated. Crystallization from EtOAc yielded the title compound in the form of greyish crystals {HPLC: Rt_{H2}=3.415 min; ESIMS: 352, 354 [(M+H)⁺, 1Br]; ¹H-NMR (360 MHz, CDCl₃): 8.19 (br, 1H), 7.75 (dd, 1H), 7.45 (ddd, 1H), 6.94 (dd, 1H), 3.99 (d, 1H), 3.92 (d, 1H), 3.81 (s, 3H), 2.09 (s, 3H)}.

### 186d) 3-(5-Bromo-2-fluoro-phenyl)-1,3-dimethyl-piperazine-2,5-dione

To a suspension of compound 186c) (353 mg, 1 mmol) in EtOH were added 2.5 ml of MeNH₂ (33% in EtOH). The mixture was stirred for 1.5 h at 50° C. and evaporated. Crystallization from TBME/hexane yielded the title compound in the form of colourless crystals {HPLC: Rt_{H2}=2.337 min; ESIMS: 315, 317 [(M+H)⁺, 1Br]; ¹H-NMR (360 MHz, DMSO-d₆): 8.67 (br, 1H), 7.66-7.57 (m, 2H), 7.24 (dd, 1H), 4.13 (d, 1H), 3.96 (d, 1H), 2.89 (s, 3H), 1.79 (s, 3H)}.

### 186e) 3-(5-Bromo-2-fluoro-phenyl)-1,3-dimethyl-5-thioxo-piperazin-2-one

A mixture of compound 186d) (158 mg, 0.5 mmol), 142 mg (0.35 mmol) of Lawesson's reagent and 2 ml of THF was stirred for 2 h at 50° C., cooled and filtered to yield the title compound in the form of colourless crystals {HPLC: Rt_{H2}=2.837 min; ESIMS: 331, 333 [(M+H)⁺, 1Br]; ¹H-NMR (600 MHz, DMSO-d₆): 11.00 (s, 1H), 7.65 (ddd, 1H), 7.60 (dd, 1H), 7.24 (dd, 1H), 4.61 (d, 1H), 4.42 (d, 1H), 2.88 (s, 3H), 1.80 (s, 3H)}.

### 186f) 5-Amino-3-(5-bromo-2-fluoro-phenyl)-1,3-dimethyl-3,6-dihydro-1H-pyrazin-2-one

To a suspension of compound 186e) (2.03 g, 6.13 mmol) in 30 ml of MeOH and 30 ml of THF were added 11.6 ml of aq NH₃ (25%) and 9.6 ml of tBuOOH (80% in water). The mixture was stirred overnight at 40° C., cooled down and treated with sodium thiosulphate to destroy excess peroxide. MeOH and THF were evaporated, the residue was extracted with EtOAc, the organic phase was extracted twice with 1N HCl, and the combined acidic layers were basified with solid K₂CO₃ and extracted with DCM. The organic extracts were dried over MgSO₄×H₂O and evaporated. The residue was stirred with TBME, and after filtration the title compound was obtained in the form of colourless crystals {HPLC: Rt_{H2}=2.096 min; ESIMS: 314, 316 [(M+H)⁺, 1Br]; ¹H-NMR (360 MHz, DMSO-d₆): 7.55 (dd, 1H), 7.49 (ddd, 1H), 7.08 (dd, 1H), 5.85 (br s, 2H), 4.11 (d, 1H), 3.90 (d, 1H), 2.88 (s, 3H), 1.59 (s, 3H)}.

### 186g) [6-(5-Bromo-2-fluoro-phenyl)-4,6-dimethyl-5-oxo-3,4,5,6-tetrahydro-pyrazin-2-yl]-carbamic acid tert-butyl ester

To a suspension of compound 1860 (995 mg, 3.17 mmol) in 12 ml of THF and 2 ml of DCM were added 0.83 ml (4.75 mmol) of DIPEA and 760 mg (3.48 mmol) of Boc₂O. The mixture was stirred overnight, diluted with TBME, washed with water and brine, dried over MgSO₄×H₂O and evaporated. The residue was purified by chromatography on silica gel (hexane/25 to 50% EtOAc) to yield the title compound in the form of a colourless foam {HPLC: Rt_{H2}=3.027 min; ESIMS: 414, 416 [(M+H)⁺, 1Br]; ¹H-NMR (360 MHz, CDCl₃; very broad signals due to rotamers): 7.60-6.70 (m, 3H), 4.60 and 4.05 (two br s, 2H), 3.00 (br s, 3H), 1.84 and 1.70 (two s, 3H), 1.40 (s, 9H)}.

### 186h) [6-(5-Amino-2-fluoro-phenyl)-4,6-dimethyl-5-oxo-3,4,5,6-tetrahydro-pyrazin-2-yl]-carbamic acid tert-butyl ester

Compound 186g) (100 mg, 0.241 mmol) and rac-trans-N,N-dimethylcyclohexane-1,2-diamine (5.2 mg, 0.036 mmol) were dissolved in 7 ml of EtOH. The mixture was treated with an aq solution of 31.4 mg (0.483 mmol) of NaN₃ and 2.4 mg (2.4 mmol) of L-(+)-ascorbic acid sodium salt, degassed, brought under a nitrogen atmosphere, treated with 4.6 mg (0.024 mmol) of CuI, stirred for 2 h at 45° C., diluted with TBME, washed with water, dried over MgSO₄×H₂O and evaporated. The residue was taken up in EtOH and stirred under a hydrogen atmosphere in the presence of 5 mg of Pd on carbon (10%), until all of the azide had been hydrogenated. The mixture was filtered over celite, the filtrate was evaporated, and the residue was purified by chromatography on silica gel (hexane/15 to 40% EtOAc) to yield the title compound in the form of a colourless foam {HPLC: Rt_{H2}=2.145 min; ESIMS: 351 [(M+H)⁺]; ¹H-NMR (360 MHz, CDCl₃; very broad signals due to rotamers): 7.60-6.40 (br m), 4.80-3.40 (br), 3.02 (s, 3H), 1.79 (br s, 3H), 1.40 (s, 9H)}.

### 186i) (6-{5-[(5-Bromo-pyridine-2-carbonyl)-amino]-2-fluoro-phenyl}-4,6-dimethyl-5-oxo-3,4,5,6-tetrahydro-pyrazin-2-yl)-carbamic acid tert-butyl ester

To an ice-cold solution of compound 186h) (67 mg, 0.192 mmol), 43 mg (0.211 mmol) of 5-bromo-pyridine-2-carboxylic acid, 34 mg (0.25 mmol) of HOAt and 48 mg (0.25 mmol) of EDC×HCl in DCM were added 0.66 ml (0.48 mmol) of Et₃N. The mixture was stirred overnight, diluted with EtOAc, washed with 5% aq NaHCO₃ solution and brine, dried over Na₂SO₄ and evaporated. The residue was purified by chromatography on silica gel (hexane/25 to 65% EtOAc) to yield the title compound in the form of a colourless foam {HPLC: Rt_{H2}=3.230 min; ESIMS: 534, 536 [(M+H)⁺, 1Br]; ¹H-NMR (360 MHz, CDCl₃; very broad signals due to rotamers): 9.78 (s, 1H), 8.60 (s, 1H), 8.11 (d, 1H), 7.98 (d, 1H), 7.80-7.60 (m, 2H), 7.10-6.90 (m, 1H), 4.65 (br, 1H), 4.08 (br, 1H), 3.02 (br s, 3H), 1.90 and 1.83 (two br s, 3H), 1.40 (s, 9H)}.

### 186j) 5-Bromo-pyridine-2-carboxylic acid [3-(6-amino-2,4-dimethyl-3-oxo-2,3,4,5-tetrahydro-pyrazin-2-yl)-4-fluoro-phenyl]-amide

A mixture of compound 186i) (67 mg, 0.126 mmol) and 3 ml of 3N HCl in MeOH was stirred for 3 h at 45° C. and then evaporated. The residue was basified with 10% aq Na₂CO₃ solution, the mixture was extracted with DCM, and the organic phase was dried over Na₂SO₄ and evaporated. The residue was purified by chromatography on silica gel (DCM/5 to 10% MeOH) to yield the title compound in the form of beige crystals {HPLC: Rt_{H2}=2.665 min; ESIMS: 434, 436 [(M+H)⁺, 1Br]; ¹H-NMR (600 MHz, DMSO-d₆): 10.72 (s, 1H), 8.81 (s, 1H), 8.33 (d, 1H), 8.09 (d, 1H), 7.98 (d, 1H), 7.85-7.80 (m, 1H), 7.04 (t, 1H), 5.74 (br s), 4.08 (br, 1H), 4.06 (d, 1H), 3.91 (d, 1H), 2.87 (s, 3H), 1.60 (s, 3H)}.

### Example 187

### 5-Bromo-pyridine-2-carboxylic acid [3-(6-amino-2,4-dimethyl-2,3,4,5-tetrahydro-pyrazin-2-yl)-4-fluoro-phenyl]-amide

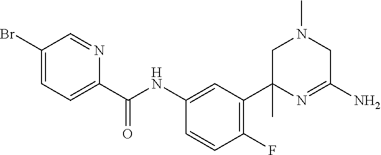 [SMILES: CN1CC(N)=NC(C)(c2cc(NC(=O)c3ccc(Br)cn3)ccc2F)C1]

### 187a) [6-(5-Amino-2-fluoro-phenyl)-4,6-dimethyl-3,4,5,6-tetrahydro-pyrazin-2-yl]-carbamic acid tert-butyl ester

A stirred solution of compound 186h) (93 mg, 0.266 mmol) in 1.5 ml of THF was treated at 4° C. with 0.6 ml of a 2M solution of LiAlH₄ in THF. The mixture was stirred for 30 min, treated with 0.32 ml (0.4 mmol) of CHCl₃, stirred for 1 h, quenched by adding 0.045 ml of water, followed by 0.045 ml of 4N aq NaOH solution and by 0.115 ml of water, dried over Na₂SO₄ and evaporated. The residue was purified by chromatography on silica gel [hexane/25 to 65% EtOAc (containing 5% MeOH)] to yield the title compound in the form of a colourless resin {HPLC: Rt_{H2}=2.365 min; ESIMS: 337 [(M+H)⁺]; ¹H-NMR (360 MHz, CDCl₃; very broad signals due to rotamers): 6.77 (dd, 1H), 6.53-6.42 (m, 2H), 3.65-3.60 (m, 2H), 3.05 (d, 1H), 2.54 (d, 1H), 2.21 (s, 3H), 1.60 (s, 3H), 1.43 (s, 9H)}.

### 187b) (6-{5-[(5-Bromo-pyridine-2-carbonyl)-amino]-2-fluoro-phenyl}-4,6-dimethyl-3,4,5,6-tetrahydro-pyrazin-2-yl)-carbamic acid tert-butyl ester

The title compound was prepared by a procedure analogous to that used in Example 186i) {HPLC: Rt_{H2}=3.418 min; ESIMS: 520, 522 [(M+H)⁺, 1Br]; ¹H-NMR (360 MHz, CDCl₃; data of major rotamer): 9.75 (br s, 1H), 8.58 (d, 1H), 8.08 (d, 1H), 7.97 (dd, 1H), 7.80-7.60 (m, 2H), 7.79-7.72 (m, 1H), 7.37 (dd, 1H), 7.03 (dd, 1H), 3.29 (d, 1H), 3.20-3.00 (br, 2H), 2.56 (d, 1H), 2.20 (s, 3H), 1.63 (s, 3H), 1.48 (s, 9H)}.

### 187c) 5-Bromo-pyridine-2-carboxylic acid [3-(6-amino-2,4-dimethyl-2,3,4,5-tetrahydro-pyrazin-2-yl)-4-fluoro-phenyl]-amide

The title compound was prepared by a procedure analogous to that used in Example 186j) {HPLC: Rt_{H2}=2.801 min; ESIMS: 420, 422 [(M+H)⁺, 1Br]; ¹H-NMR (600 MHz, DMSO-d₆): 10.52 (s, 1H), 8.89 (s, 1H), 8.32 (dd, 1H), 8.07 (dd, 1H), 7.85 (m, 1H), 7.72 (m, 1H), 7.09 (dd, 1H), 5.70-5.52 (br, 1H), 2.77 (d, 1H), 2.68 (d, 1H), 2.55-2.45 (m, 2H), 2.10 (s, 3H), 1.43 (s, 3H)}.

### Example 188

### 5-Bromo-pyridine-2-carboxylic acid [3-(6-amino-2,4-dimethyl-3-oxo-2,3,4,5-tetrahydro-pyrazin-2-yl)-phenyl]-amide

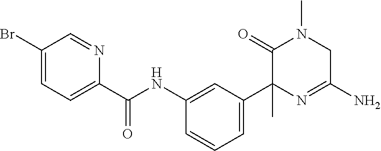 [SMILES: CN1CC(N)=NC(C)(c2cccc(NC(=O)c3ccc(Br)cn3)c2)C1=O]

The title compound was prepared by procedures analogous to those used in Example 186 {HPLC: Rt_{H2}=2.661 min; ESIMS: 416, 418 [(M+H)⁺, 1Br]; ¹H-NMR (600 MHz, DMSO-d₆): 10.55 (s, 1H), 8.86 (s, 1H), 8.33 (dd, 1H), 8.08 (dd, 1H), 7.83 (s, 1H), 7.74 (d, 1H), 7.28 (t, 1H), 7.10 (d, 1H), 6.15-6.00 (br, 2H), 3.82 (d, 1H), 3.78 (d, 1H), 2.80 (s, 3H), 1.55 (s, 3H)}.

### Example 189

### 5-Bromo-pyridine-2-carboxylic acid [3-(6-amino-2,4-dimethyl-5-oxo-2,3,4,5-tetrahydro-pyrazin-2-yl)-4-fluoro-phenyl]-amide hydrochloride

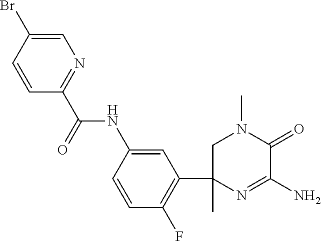 [SMILES: CN1CC(C)(c2cc(NC(=O)c3ccc(Br)cn3)ccc2F)N=C(N)C1=O]

### 189a) 2-Amino-2-(5-bromo-2-fluoro-phenyl)-propan-1-ol

A stirred suspension of compound 186a) (10.0 g, 38.0 mmol) in 110 ml of THF was treated dropwise with borane dimethyl sulfide (12.08 ml, 114 mmol) and then heated to reflux. The mixture was stirred for 5 h, cooled down, carefully quenched by adding dropwise 25 ml of MeOH, followed by 12 ml of 4N HCl and by 100 ml of MeOH, concentrated in vacuo, diluted with 200 ml of MeOH, concentrated, diluted with water, basified with 10% aq Na₂CO₃ solution and extracted three times with DCM. The organic phases were dried over K₂CO₃ and evaporated to yield the title compound in the form of a colourless solid {HPLC: Rt_{H1}=2.540 min; ESIMS: 248, 250 [(M+H)⁺, 1Br]; ¹H-NMR (360 MHz, DMSO-d₆): 7.84 (dd, 1H), 7.49 (ddd, 1H), 7.34 (dd, 1H), 4.84 (br t, 1H), 3.64 (br dd, 1H), 3.50 (br dd, 1H), 2.15 (br s, 2H), 1.35 (s, 3H)}.

### 189b) [1-(5-Bromo-2-fluoro-phenyl)-2-hydroxy-1-methyl-ethyl]-carbamic acid tert-butyl ester

Compound 189a) (17.98 g, 72.5 mmol) and 23 g (109 mmol) of Boc₂O were dissolved in 43 ml of dioxane. The mixture was treated with 43 ml of saturated aq NaHCO₃ solution, stirred overnight, diluted with water and extracted with TBME. The organic phase was washed with brine, dried over MgSO₄×H₂O, evaporated and diluted with hexane to yield the title compound in the form of colourless crystals {HPLC: Rt_{H3}=2.906 min; ESIMS: 370, 372 [(M+Na)⁺, 1Br]; ¹H-NMR (360 MHz, CDCl₃): 7.47 (dd, 1H), 7.40 (ddd, 1H), 6.93 (dd, 1H), 5.24 (br s, 1H), 4.15 (br d, 1H), 3.88 (d, 1H), 1.59 (s, 3H), 1.48 (br s, 9H)}.

### 189c) 4-(5-Bromo-2-fluoro-phenyl)-4-methyl-2,2-dioxo-2lambda*6*-[1,2,3]oxathiazolidine-3-carboxylic acid tert-butyl ester

A solution of compound 189b) (22.46 g, 64.5 mmol) in 645 ml of DCM was added dropwise at 0° C. to a solution of 9.42 ml (129 mmol) of thionyl chloride in 26.1 ml (330 mmol) of pyridine. The mixture was slowly warmed to 25° C., stirred for 16 h, treated with 1N HCl and extracted with TBME. The organic phase was treated with charcoal, filtered over celite, evaporated, taken up in 130 ml of ACN, treated at 0 to 5° C. with 7.3 mg (0.032 mmol) of Ru(III)C₁₋₃ hydrate, then with 13.8 g of NaIO₄ and with 130 ml of water, stirred for 1 h at 25° C., diluted with water and extracted with DCM. The extract was washed with brine, dried over MgSO₄×H₂O, treated with charcoal, filtered over celite, evaporated and diluted with hexane to yield the title compound in the form of a colourless crystalline solid {HPLC: Rt_{H4}=3.019 min; ESIMS: 841, 843, 845 [(2M+Na)⁺, 1Br]; ¹H-NMR (360 MHz, CDCl₃): 7.52 (ddd, 1H), 7.43 (dd, 1H), 7.05 (dd, 1H), 4.72 (d, 1H), 4.48 (d, 1H), 2.05 (s, 3H), 1.55 (s, 9H)}.

### 189d) [1-(5-Bromo-2-fluoro-phenyl)-1-methyl-2-methylamino-ethyl]-carbamic acid tert-butyl ester

A mixture of compound 189c) (2.0 g, 4.88 mmol) and 12.14 ml (98 mmol) of MeNH₂ (33% in EtOH) was stirred for 18 h at 25° C., treated with 10 ml of 2N HCl, stirred for 1 h, neutralized with 10% aq NaHCO₃ solution and extracted with DCM. The organic phase was dried over K₂CO₃ and purified by chromatography on silica gel (DCM/1 to 2% MeOH) to yield the title compound in the form of a colourless resin {HPLC: Rt_{H2}=2.918 min; ESIMS: 361, 363 [(M+H)⁺, 1Br]; ¹H-NMR (360 MHz, CDCl₃): 7.80-7.67 (m, 1H), 7.41 (ddd, 1H), 6.96 (dd, 1H), 3.85-3.55 (m, 2H), 2.75 and 2.69 (two s, 3H; two rotamers), 2.05 (s, 3H), 1.68 and 1.45 (two s, 9H; two rotamers)}.

### 189e) N-[2-(5-Bromo-2-fluoro-phenyl)-2-tert-butoxycarbonylamino-propyl]-N-methyl-oxalamic acid methyl ester

A mixture of compound 189d) (1.06 g, 2.93 mmol), 0.67 ml (3.81 mmol) of DIPEA and DCM was treated at −78° C. dropwise with 0.3 ml (3.32 mmol) of monomethyl oxalylchloride, allowed to warm to 25° C., diluted with TBME, washed with 1N HCl and brine, dried over MgSO₄×H₂O and purified by chromatography on silica gel (hexane/EtOAc 3:1) to yield the title compound in the form of a colourless solid {HPLC: Rt_{H3}=3.445 min; ESIMS: 469, 471 [(M+Na)⁺, 1Br]; ¹H-NMR (360 MHz, CDCl₃; major rotamer): 9.90 (br s, 1H), 7.45-7.36 (m, 2H), 6.95 (dd, 1H), 3.92 (s, 3H), 3.65 (br s, 2H), 2.65 (s, 3H), 1.93 (s, 3H), 1.57 (s, 9H)}.

### 189f) 5-(5-Bromo-2-fluoro-phenyl)-1,5-dimethyl-piperazine-2,3-dione

A mixture of compound 189e) (1.16 g) and 13 ml of 4N HCl in dioxane was slightly warmed to 25° C., stirred for 1 h and evaporated. The residue was taken up in saturated aq NaHCO₃ solution and extracted with DCM. The organic phase was dried over MgSO₄×H₂O, evaporated and diluted with TBME/hexane to yield the title compound in the form of colourless crystals {HPLC: Rt_{H2}=2.566 min; ESIMS: 315, 317 [(M+H)⁺, 1Br]; ¹H-NMR (360 MHz, DMSO-d₆): 9.38 (br s, 1H), 7.63 (ddd, 1H), 6.30 (dd, 1H), 4.00 (d, 1H), 3.88 (d, 1H), 2.81 (s, 3H), 1.55 (s, 3H)}.

### 189g) 5-(5-Bromo-2-fluoro-phenyl)-1,5-dimethyl-3-thioxo-piperazin-2-one

To a solution of compound 1890 (674 mg, 2.139 mmol) in pyridine were added 475 mg (2.139 mmol) of phosphorous pentasulfide. The mixture was stirred for 3 h at 80° C., cooled down, diluted with EtOAc, washed with 1N HCl, 5% aq NaHCO₃ solution and brine, dried over MgSO₄×H₂O and purified by chromatography on silica gel (hexane/35 to 50% EtOAc) to yield the title compound in the form of a yellow foam {HPLC: Rt_{H2}=2.783 min; ESIMS: 331, 333 [(M+H)⁺, 1Br]; ¹H-NMR (360 MHz, DMSO-d₆): 11.66 (s, 1H), 7.64 (ddd, 1H), 7.28-6.36 (m, 2H), 4.02 (d, 1H), 3.94 (d, 1H), 2.85 (s, 3H), 1.61 (s, 3H)}.

### 189h) 3-Amino-5-(5-bromo-2-fluoro-phenyl)-1,5-dimethyl-5,6-dihydro-1H-pyrazin-2-one

A mixture of compound 189g) (545 g, 1.646 mmol) in 7 ml of a 7M methanolic solution of NH₃ was stirred for 18 h at 25° C., evaporated and purified by chromatography on silica gel (DCM/0.5 to 5% EtOH) to yield the title compound in the form of a colourless solid {HPLC: Rt_{H2}=2.402 min; ESIMS: 314, 316 [(M+H)⁺, 1Br]; ¹H-NMR (360 MHz, DMSO-d₆): 7.84 (dd, 1H), 7.53 (ddd, 1H), 7.21 (dd, 1H), 6.49 (br s, 2H), 3.78 (d, 1H), 3.68 (d, 1H), 2.92 (s, 3H), 1.45 (s, 3H)}.

### 189i) 5-Bromo-pyridine-2-carboxylic acid [3-(6-amino-2,4-dimethyl-5-oxo-2,3,4,5-tetrahydro-pyrazin-2-yl)-4-fluoro-phenyl]-amide hydrochloride

The title compound was prepared by procedures analogous to those used in example 186, starting from compound 189h) {HPLC: Rt_{H2}=2.787 min; ESIMS: 434, 436 [(M+H)⁺, 1Br]; ¹H-NMR (600 MHz, DMSO-d₆): 11.22 (s, 1H), 10.97 (s, 1H), 9.78 (br s, 1H), 9.48 (br s, 1H), 8.87 (s, 1H), 8.34 (dd, 1H), 8.08 (d, 1H), 7.99-7.93 (m, 2H), 7.32 (dd, 1H), 4.10 (d, 1H), 3.98 (d, 1H), 2.97 (s, 3H), 1.68 (s, 3H)}.

### Example 190

### 5-Chloro-pyridine-2-carboxylic acid [3-(6-amino-2,4-dimethyl-5-oxo-2,3,4,5-tetrahydro-pyrazin-2-yl)-4-fluoro-phenyl]-amide hydrochloride

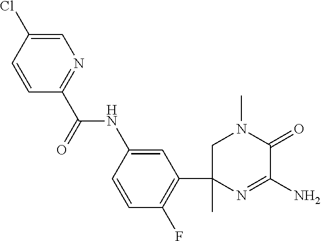 [SMILES: CN1CC(C)(c2cc(NC(=O)c3ccc(Cl)cn3)ccc2F)N=C(N)C1=O]

The title compound was prepared by procedures analogous to those used in the examples hereinbefore {HPLC: Rt_{H2}=2.723 min; ESIMS: 390 [(M+H)⁺]; ¹H-NMR (600 MHz, DMSO-d₆): 11.28 (s, 1H), 10.97 (s, 1H), 9.79 (br s, 1H), 9.48 (br, 1H), 8.79 (d, 1H), 8.21 (dd, 1H), 8.15 (d, 1H), 7.99-7.93 (m, 2H), 7.32 (dd, 1H), 4.11 (d, 1H), 3.98 (d, 1H), 2.95 (s, 3H), 1.68 (s, 3H)}.

### Example 191

### 5-Bromo-pyridine-2-carboxylic acid [3-(6-amino-4-ethyl-2-methyl-5-oxo-2,3,4,5-tetrahydro-pyrazin-2-yl)-4-fluoro-phenyl]-amide hydrochloride

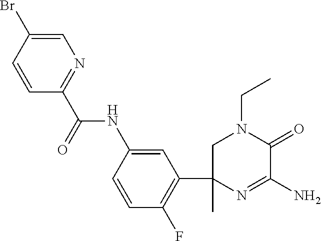 [SMILES: CCN1CC(C)(c2cc(NC(=O)c3ccc(Br)cn3)ccc2F)N=C(N)C1=O]

The title compound was prepared by procedures analogous to those used in the examples hereinbefore {HPLC: Rt_{H2}=2.913 min; ESIMS: 448, 450 [(M+H)⁺, 1Br]; ¹H-NMR (600 MHz, DMSO-d₆): 11.23 (br s, 1H), 10.98 (s, 1H), 9.81 (br s, 1H), 9.58 (br s, 1H), 8.87 (d, 1H), 8.34 (dd, 1H), 8.07 (d, 1H), 8.02-7.98 (m, 1H), 7.92 (dd, 1H), 7.33 (dd, 1H), 4.11 (d, 1H), 3.97 (d, 1H), 3.48-3.44 (m, 1H), 3.30-3.26 (m, 1H), 1.70 (s, 3H), 0.80 (t, 3H)}.

### Example 192

### 3,5-Dichloro-pyridine-2-carboxylic acid [3-(6-amino-2,4-dimethyl-5-oxo-2,3,4,5-tetrahydro-pyrazin-2-yl)-4-fluoro-phenyl]-amide hydrochloride

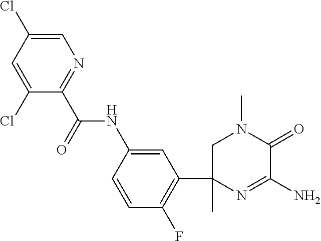 [SMILES: CN1CC(C)(c2cc(NC(=O)c3ncc(Cl)cc3Cl)ccc2F)N=C(N)C1=O]

The title compound was prepared by procedures analogous to those used in the examples hereinbefore {HPLC: Rt_{H2}=2.732 min; ESIMS: 424 [(M+H)⁺]; ¹H-NMR (600 MHz, DMSO-d₆): 11.27 (br s, 1H), 10.97 (s, 1H), 9.80 (br s, 1H), 9.52 (br s, 1H), 8.74 (d, 1H), 8.48 (d, 1H), 7.79 (dd, 1H), 7.73 (dd, 1H), 7.35 (dd, 1H), 4.11 (d, 1H), 3.98 (d, 1H), 2.95 (s, 3H), 1.69 (s, 3H)}.

### Example 193

### 3,5-Dichloro-pyridine-2-carboxylic acid [3-(6-amino-4-ethyl-2-methyl-5-oxo-2,3,4,5-tetrahydro-pyrazin-2-yl)-4-fluoro-phenyl]-amide hydrochloride

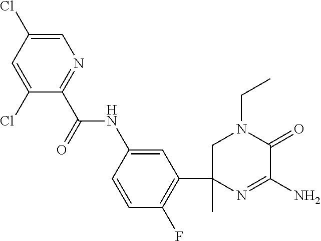 [SMILES: CCN1CC(C)(c2cc(NC(=O)c3ncc(Cl)cc3Cl)ccc2F)N=C(N)C1=O]

The title compound was prepared by procedures analogous to those used in the examples hereinbefore {HPLC: Rt_{H2}=2.849 min; ESIMS: 438 [(M+H)⁺]; ¹H-NMR (600 MHz, DMSO-d₆): 11.27 (br s, 1H), 10.97 (s, 1H), 9.85 (br s, 1H), 9.63 (br s, 1H), 8.74 (d, 1H), 8.47 (d, 1H), 7.85-7.81 (m, 1H), 7.67 (dd, 1H), 7.34 (dd, 1H), 4.10 (d, 1H), 3.93 (d, 1H), 3.48-3.39 (m, 1H), 3.31-3.22 (m, 1H), 1.71 (s, 3H), 0.80 (t, 3H)}.

### Example 194

### 5-Bromo-pyridine-2-carboxylic acid [3-(6-amino-4-isopropyl-2-methyl-5-oxo-2,3,4,5-tetrahydro-pyrazin-2-yl)-4-fluoro-phenyl]-amide hydrochloride

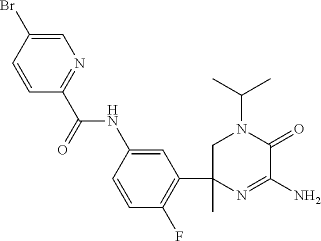 [SMILES: CC(C)N1CC(C)(c2cc(NC(=O)c3ccc(Br)cn3)ccc2F)N=C(N)C1=O]

The title compound was prepared by procedures analogous to those used in the examples hereinbefore {HPLC: Rt_{H2}=3.006 min; ESIMS: 462, 464 [(M+H)⁺, 1Br]; ¹H-NMR (600 MHz, DMSO-d₆): 11.19 (br s, 1H), 10.98 (s, 1H), 9.84 (br s, 1H), 9.62 (br s, 1H), 8.87 (d, 1H), 8.33 (dd, 1H), 8.03-7.98 (m, 1H), 7.90 (dd, 1H), 7.32 (dd, 1H), 4.41 (heptett, 1H), 3.88 (s, 2H), 1.75 (s, 3H), 1.07 (d, 3H), 0.66 (d, 3H)}.

### Example 195

### 3,5-Dichloro-pyridine-2-carboxylic acid {3-[6-amino-4-(2-methoxy-ethyl)-2-methyl-5-oxo-2,3,4,5-tetrahydro-pyrazin-2-yl]-4-fluoro-phenyl}-amide hydrochloride

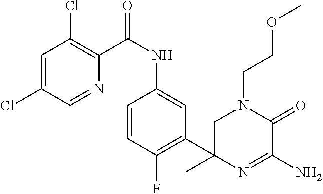 [SMILES: COCCN1CC(C)(c2cc(NC(=O)c3ncc(Cl)cc3Cl)ccc2F)N=C(N)C1=O]

The title compound was prepared by procedures analogous to those used in the examples hereinbefore {HPLC: Rt_{H2}=2.783 min; ESIMS: 468 [(M+H)⁺]; ¹H-NMR (600 MHz, DMSO-d₆): 11.22 (br s, 1H), 10.93 (s, 1H), 9.87 (br s, 1H), 9.60 (br s, 1H), 8.74 (d, 1H), 8.47 (d, 1H), 7.86-7.82 (m, 1H), 7.65 (dd, 1H), 7.34 (dd, 1H), 4.11 (d, 1H), 4.04 (d, 1H), 3.63-3.58 (m, 1H), 3.42-3.37 (m, 1H), 3.30-3.25 (m, 1H), 3.20-3.15 (m, 1H), 3.00 (s, 3H), 1.69 (s, 3H)}.

### Example 196

### 5-Bromo-pyridine-2-carboxylic acid {3-[6-amino-4-(2-methoxy-ethyl)-2-methyl-5-oxo-2,3,4,5-tetrahydro-pyrazin-2-yl]-4-fluoro-phenyl}-amide hydrochloride

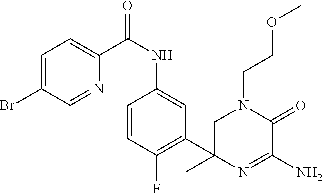 [SMILES: COCCN1CC(C)(c2cc(NC(=O)c3ccc(Br)cn3)ccc2F)N=C(N)C1=O]

The title compound was prepared by procedures analogous to those used in the examples hereinbefore {HPLC: Rt_{H2}=2.802 min; ESIMS: 478, 480 [(M+H)⁺, 1Br]; ¹H-NMR (600 MHz, DMSO-d₆): 11.20 (br s, 1H), 10.96 (s, 1H), 9.81 (br s, 1H), 9.57 (br s, 1H), 8.87 (d, 1H), 8.33 (dd, 1H), 8.07 (d, 1H), 8.03-7.98 (m, 1H), 7.91 (dd, 1H), 7.32 (dd, 1H), 4.09 (d, 1H), 4.04 (d, 1H), 3.63-3.58 (m, 1H), 3.45-3.40 (m, 1H), 3.30-3.25 (m, 1H), 3.20-3.15 (m, 1H), 3.00 (s, 3H), 1.70 (s, 3H)}.

### Example 197

### 5-Bromo-pyridine-2-carboxylic acid {3-[6-amino-2-methyl-4-(1-methyl-1H-pyrazol-4-yl)-5-oxo-2,3,4,5-tetrahydro-pyrazin-2-yl]-4-fluoro-phenyl}-amide hydrochloride

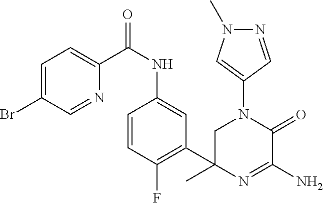 [SMILES: Cn1cc(N2CC(C)(c3cc(NC(=O)c4ccc(Br)cn4)ccc3F)N=C(N)C2=O)cn1]

The title compound was prepared by procedures analogous to those used in the examples hereinbefore {HPLC: Rt_{H2}=2.777 min; ESIMS: 500, 502 [(M+H)⁺, 1Br]; ¹H-NMR (600 MHz, DMSO-d₆): 11.40 (br s, 1H), 10.95 (s, 1H), 9.98 (br d, 1H), 9.68 (br d, 1H), 8.86 (d, 1H), 8.34 (dd, 1H), 8.08 (s, 1H), 8.06 (d, 1H), 7.98-7.92 (m, 2H), 7.67 (s, 1H), 7.29 (dd, 1H), 4.47 (s, 2H), 3.79 (s, 3H), 1.78 (s, 3H)}.

### Example 198

### 5-Bromo-pyridine-2-carboxylic acid [3-(6-amino-2-methyl-5-oxo-4-pyridin-3-yl-2,3,4,5-tetrahydro-pyrazin-2-yl)-4-fluoro-phenyl]-amide hydrochloride

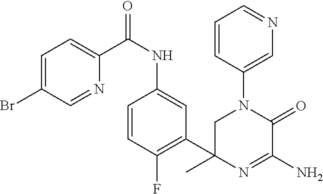 [SMILES: CC1(c2cc(NC(=O)c3ccc(Br)cn3)ccc2F)CN(c2cccnc2)C(=O)C(N)=N1]

The title compound was prepared by procedures analogous to those used in the examples hereinbefore {HPLC: Rt_{H2}=2.667 min; ESIMS: 497, 499 [(M+H)⁺, 1Br]; ¹H-NMR (600 MHz, DMSO-d₆): 11.56 (br s, 1H), 10.99 (s, 1H), 10.08-10.04 (m, 1H), 9.82-9.78 (m, 1H), 8.87 (d, 1H), 8.54 (d, 1H), 8.38 (s, 1H), 8.34 (dd, 1H), 8.07 (s, 1H), 8.05-7.98 (m, 2H), 7.63 (d, 1H), 7.54 (dd, 1H), 7.34 (dd, 1H), 4.60 (d, 1H), 4.31 (d, 1H), 1.80 (s, 3H)}.

### Example 199

### 5-Cyano-pyridine-2-carboxylic acid [3-(6-amino-4-ethyl-2-methyl-5-oxo-2,3,4,5-tetrahydro-pyrazin-2-yl)-4-fluoro-phenyl]-amide hydrochloride

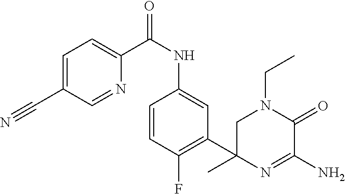 [SMILES: CCN1CC(C)(c2cc(NC(=O)c3ccc(C#N)cn3)ccc2F)N=C(N)C1=O]

The title compound was prepared by procedures analogous to those used in the examples hereinbefore {HPLC: Rt_{H2}=2.635 min; ESIMS: 395 [(M+H)⁺]; ¹H-NMR (600 MHz, DMSO-d₆): 11.22 (br s, 1H), 11.14 (s, 1H), 9.81 (br s, 1H), 9.51 (br s, 1H), 9.19 (s, 1H), 8.59 (dd, 1H), 8.27 (d, 1H), 8.03-7.99 (m, 1H), 7.95-7.91 (m, 1H), 7.34 (dd, 1H), 4.10 (d, 1H), 3.95 (d, 1H), 3.47-3.38 (m, 1H), 3.30-3.21 (m, 1H), 1.70 (s, 3H), 0.79 (t, 3H)}.

### Example 200

### 5-Bromo-pyridine-2-carboxylic acid [3-(6-amino-5-ethyl-2,4-dimethyl-3-oxo-2,3,4,5-tetrahydro-pyrazin-2-yl)-phenyl]-amide (9:1 mixture of two diastereomers)

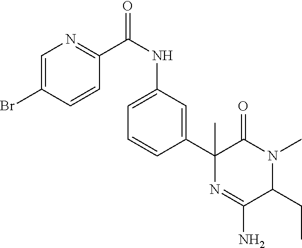 [SMILES: CCC1C(N)=NC(C)(c2cccc(NC(=O)c3ccc(Br)cn3)c2)C(=O)N1C]

The title compound was prepared by procedures analogous to those used in the examples hereinbefore {HPLC: Rt_{H2}=2.866 min (major diastereomer); ESIMS: 444, 446 [(M+H)⁺, 1Br]; ¹H-NMR (600 MHz, DMSO-d₆; major diastereomer): 10.53 (s, 1H), 8.85 (s, 1H), 8.32 (dd, 1H), 8.07 (dd, 1H), 7.97 (s, 1H), 7.68 (d, 1H), 7.26-7.20 (m, 2H), 5.98 (s, 2H), 3.79 (t, 1H), 2.87 (s, 3H), 1.42-1.29 (m, 2H), 0.67 (t, 3H)}.

### Example 201

### 5-Bromo-pyridine-2-carboxylic acid [3-(6-amino-2-difluoromethyl-2,3,4,5-tetrahydro-pyrazin-2-yl)-4-fluoro-phenyl]-amide

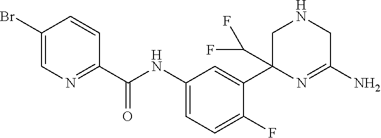 [SMILES: NC1=NC(c2cc(NC(=O)c3ccc(Br)cn3)ccc2F)(C(F)F)CNC1]

### 201a) 1-(5-Bromo-2-fluoro-phenyl)-ethanone

A solution of 711 ml (5.03 mol) diisopropyl amine in 8 L THF was cooled to −80° C. A 2.5 M solution of BuLi in hexanes (2.01 L, 5.03 mol) was added over a period of 15 minutes. After 30 minutes a solution of 500 ml of 4-bromo-1-fluoro benzene (4.574 mol) was added while keeping the temperature below −65° C. After stirring for 2.5 h at −65° C. the mixture was cooled to −80° C. and 681g ethyl difluoro acetate (5.488 mol) were added, while keeping the temperature below −65° C. The mixture was warmed to −40° C. and then quenched by pouring the mixture onto 15 L ice-cold 1M HCl and 15 L TBME. The phases were separated and the organic phase was washed with 10% aq NaHCO₃ and brine. The extract was dried with sodium sulfate, filtered, concentrated and purified by distillation at 0.1 mbar. The fraction boiling at 67-72° C. was collected. Yield 856 g (74%) of a pale yellow liquid.

¹H-NMR (CDCl₃, 360 MHz): 8.09 (dd, 1H), 7.82-7.77 (m, 1H), 7.17 (t, 1H), 6.45 (t, 1H, CHF₂).

### 201b) [1-(5-Bromo-2-fluoro-phenyl)-2,2-difluoro-eth-(Z)-ylidene]-carbamic acid tert-butyl ester

A mixture of 675 g (2.668 mol) 1-(5-bromo-2-fluoro-phenyl)-ethanone (compound 201a) and 1007 g (2.668 mol) N-tert-butyloxycarbonyl-triphenyliminophosphorane were suspended in 505 ml toluene and heated at 105° C. for 4 h. After cooling down to 80° C. 3 L heptane were added and the mixture was stirred at 25° C. overnight. Crystallized triphenylphosphine oxide was removed by filtration and the filtrate was purified via chromatography on silica gel (heptane/5% EtOAc)

¹H-NMR (CDCl₃, 360 MHz): 7.90-7.84 (m, 1H), 7.75-7.67 (m, 1H), 7.47 (t, 1H), 6.88 (t, 1H, CHF₂), 1.30 (br s, 9H).

### 201c) [1-(5-Bromo-2-fluoro-phenyl)-2,2-difluoro-1-nitromethyl-ethyl]-carbamic acid tert-butyl ester

At 25° C. a solution of 7.5 g (21.3 mmol) compound 201b in 30 ml of nitromethane was treated with 0.2 ml DBU (1.3 mmol). After 2 h the mixture was diluted with 50 ml TBME, washed with 1N HCl and water. The organic phase was evaporated and the residue was crystallized from TBME/hexane to give the title compound as white crystals. HPLC: Rt_{H3}=3.418 min; ESIMS: 435, 437 [(M+Na)⁺, 1Br]; ¹H-NMR (CDCl₃, 400 MHz): 7.56-7.48 (m, 2H), 7.01 (dd, 1H), 6.56 (t, 1H, CHF₂), 5.53 (br d, 1H), 5.38 (br d, 1H), 1.45 (br s, 9H).

### 201d) [1-Aminomethyl-1-(5-bromo-2-fluoro-phenyl)-2,2-difluoro-ethyl]-carbamic acid tert-butyl ester

Zinc dust (2.74 g, 41.8 mmol) was suspended in 20 ml acetic acid and a solution of 2.47 g (5.98 mmol) compound 201c in 20 ml acetic acid was added dropwise keeping the temperature below 40° C. After 2 h stirring at rt the mixture was filtered over celite. The filter cake was washed with MeOH, the filtrated was basified with 10% aq Na₂CO₃ and extracted with EtOAc. The organic phase was washed with brine, dried with sodium sulfate and evaporated. The product was obtained as white crystals (from hexane).

HPLC: Rt_{H5}=2.177 min; ESIMS: 383, 385 [(M+H)⁺, 1Br];

¹H-NMR (CDCl₃, 360 MHz): 7.55 (dd, 1H), 7.46 (ddd, 1H), 7.00 (dd, 1H), 6.42 (t, 1H, CHF₂), 5.78 (br s, 2H), 3.49 (br d, 1H), 3.87 (br d, 1H), 1.65-1.25 (br, 9H).

### 201e) [2-(5-Bromo-2-fluoro-phenyl)-2-tert-butoxycarbonylamino-3,3-difluoro-propylamino]-acetic acid tert-butyl ester

A mixture of 887 mg (2.315 mmol) compound 201d, 451 mg (2.315 mmol) tert-butyl bromoacetate and 1.21 ml (6.94 mmol) DIPEA in 8 ml ACN was stirred at 80° C. for 1.5 h. The mixture was diluted with EtOAc, washed with 1N HCl, 5% aq NaHCO₃ and water. The organic phase was dried with sodium sulfate and the product was purified via chromatography on silica gel (heptane/15% EtOAc) to give the title compound as a colorless resin. TLC: Rf 0.21 (EtOAc/heptane 1:6; HPLC: Rt_{H3}=2.785 min; ESIMS: 497, 499 [(M+H)⁺, 1Br]; ¹H-NMR (CDCl₃, 360 MHz): 7.51 (dd, 1H), 7.33 (ddd, 1H), 6.87 (dd, 1H), 6.44 (t, 1H, CHF₂), 6.04 (br s, 1H), 3.28-3.02 (m, 4H), 1.39 (s, 9H), 1.32 (br s, 9H).

### 201f) 6-(5-Bromo-2-fluoro-phenyl)-6-difluoromethyl-piperazin-2-one

A solution of 1.0 g (2.011 mmol) compound 201e in 8 ml DCM was treated with 5 ml 4N HCl in dioxane. After 4 h the mixture was evaporated, dissolved in 10 ml MeOH and left standing overnight. The MeOH was partially removed and crystallization initiated by careful addition of TBME. The hydrochloride salt of the title compound was isolated as white crystals. TLC (free base): Rf 0.39 (EtOAc); HPLC: Rt_{H1}=2.543 min; ESIMS: 323, 325 [(M+H)⁺, 1Br]; ¹H-NMR (HCl salt, dmso-d6, 360 MHz): 10.2-9.7 (br, 2H), 7.78-7.73 (m, 2H), 7.35 (dd, 1H), 6.65 (t, 1H, CHF₂), 3.90-3.68 (m, 4H).

### 201g) 3-(5-Bromo-2-fluoro-phenyl)-3-difluoromethyl-5-oxo-piperazine-1-carboxylic acid tert-butyl ester

A suspension of 300 mg (0.834 mmol) compound 201f and 273 mg (1.25 mmol) Boc2O in 4 ml ACN was treated with 0.4 ml (2.25 mmol) DIPEA. The mixture was stirred overnight, diluted with EtOAc, washed with 1N HCl, brine and 10% aq NaHCO₃, and dried with MgSO₄.H₂O. The crude product was purified via chromatography on silica gel (heptane/0-50% EtOAc) to give the title compound as a white solid. HPLC: Rt_{H3}=2.776 min; ESIMS: 445, 447 [(M+Na)⁺, 1Br]; ¹H-NMR (CDCl₃, 360 MHz, broad signals due to rotamers): 7.60-7.52 (m, 2H), 7.09 (dd, 1H), 6.6-6.1 (m, 3H), 4.6-3.63 (m, 4H), 1.35 and 1.29 (br s, 9H).

### 201h) 3-(5-Bromo-2-fluoro-phenyl)-3-difluoromethyl-5-thioxo-piperazine-1-carboxylic acid tert-butyl ester

A mixture of 329 mg (0.777 mmol) compound 201g and 283 mg (0.7 mmol) Lawesson's reagent in 4 ml THF was stirred overnight. The mixture was concentrated and purified via chromatography on silica gel (heptane/0-15% EtOAc) to give the title compound as a white solid. HPLC: Rt_{H3}=3.317 min; ESIMS: 461, 463 [(M+Na)⁺, 1Br]; ¹H-NMR (CDCl₃, 360 MHz, broad signals due to rotamers): 8.45-8.32 (br, 1H), 7.62-7.54 (br, 1H), 7.44 (dd, 1H), 7.11 (dd, 1H), 6.6-6.2 (br, 1H), 5.1-4.3 (m, 3H), 3.75-3.65 (m, 1H), 1.35 and 1.29 (br s, 9H).

### 201i) 5-Amino-3-(5-bromo-2-fluoro-phenyl)-3-difluoromethyl-3,6-dihydro-2H-pyrazine-1-carboxylic acid tert-butyl ester

A solution of 310 mg (0.706 mmol) of compound 201h in 4 ml 7M NH3/MeOH was stirred at rt for 15 h. The mixture was evaporated, dissolved in EtOAc, washed with aq NaHCO₃ and brine. The org phase was dried with Na₂SO₄ and evaporated to give the title compound as a white solid, pure enough for further synthesis. HPLC: Rt_{H5}=2.270 min; ESIMS [M+H]⁺=422/424(1Br); ¹H-NMR (CDCl₃, 360 MHz, broadened signals, rotamers): 7.7-7.58 (m, 1H), 7.38-7.30 (m, 1H), 6.88 (dd, 1H), 6.03 (br t, 1H, CHF2, major rotamer), 4.7 (br, 2H), 4.10-3.56 (m, 4H), 1.26 (br s, 9H, major rotamer). TLC (Hexane, EtOAc 1:1) Rf 0.42

### 201j) 3-(5-Bromo-2-fluoro-phenyl)-5-tert-butoxycarbonylamino-3-difluoromethyl-3,6-dihydro-2H-pyrazine-1-carboxylic acid tert-butyl ester

To an ice-cold solution of 290 mg (0.687 mmol) compound 201i in 4 ml ACN were added 225 mg (1.02 mmol) Boc₂O and 0.205 ml (1.17 mmol) DIPEA. The mixture was stirred for 4 h at rt. Then the mixture was diluted with TBME and washed with 5% aq NaHCO₃. The organic phase was dried with MgSO₄.H₂O, filtered and concentrated. Purification by chromatography on silica gel (hexane/0-25% EtOAc) gave the desired product as a colorless foam.

TLC: Rf (Hexane/EtOAc 6:1)=0.27. HPLC: Rt_{H6}=2.724 min; ESIMS [M+H]⁺=522/524(1Br);

¹H-NMR (CDCl3, 360 MHz): Spectrum uninterpretable due to complex rotamer mixture.

### 201k) 3-(5-Amino-2-fluoro-phenyl)-5-tert-butoxycarbonylamino-3-difluoromethyl-3,6-dihydro-2H-pyrazine-1-carboxylic acid tert-butyl ester

Compound 201j (350 mg, 0.671 mmol) and rac-trans-N,N-dimethylcyclohexane-1,2-diamine (28.6 mg, 0.201 mmol) were dissolved in 2.5 ml of EtOH. The mixture was treated with an aq solution of 174 mg (2.68 mmol) of NaN₃ and 26.5 mg (0.134 mmol) of L-(+)-ascorbic acid sodium salt, degassed, brought under a nitrogen atmosphere, treated with 25.5 mg (0.134 mmol) of CuI, stirred for 30 min at 70° C., diluted with TBME, washed with water, dried over MgSO₄.H₂O and evaporated. The residue was taken up in EtOH and stirred under a hydrogen atmosphere in the presence of 5 mg of Pd on carbon (10%), until all of the azide had been hydrogenated. The mixture was filtered over celite, the filtrate was evaporated, and the residue was purified by chromatography on silica gel (hexane/15 to 70% EtOAc) to yield the title compound in the form of a colourless foam. HPLC: Rt_{H3}=2.411 min; ESIMS: 459 [(M+H)⁺]; ¹H-NMR (360 MHz, CDCl₃; very broad signals due to rotamers): 7.30-5.90 (br m), 4.80-3.40 (br), 1.50-1.10 (br m).

### 201l) 3-{5-[(5-Bromo-pyridine-2-carbonyl)-amino]-2-fluoro-phenyl}-5-tert-butoxycarbonylamino-3-difluoromethyl-3,6-dihydro-2H-pyrazine-1-carboxylic acid tert-butyl ester

To an ice-cold solution of compound 201k (70 mg, 0.153 mmol), 34 mg (0.168 mmol) of 5-bromo-pyridine-2-carboxylic acid, 27 mg (0.198 mmol) of HOAt and 44 mg (0.23 mmol) of EDC×HCl in DCM were added 0.053 ml (0.382 mmol) of Et₃N. The mixture was stirred overnight, diluted with EtOAc, washed with 5% aq NaHCO₃ solution and brine, dried over Na₂SO₄ and evaporated. The residue was purified by chromatography on silica gel (heptane/EtOAc 0 to 40% EtOAc) to yield the title compound in the form of a colourless foam {HPLC: Rt_{H6}=2.713 min; ESIMS: 642, 644 [(M+H)⁺, 1Br]; ¹H-NMR (360 MHz, CDCl₃; very broad signals due to rotamers): 9.75 (s, 1H), 8.61 (s, 1H), 8.12-7.95 (m), 7.40-7.0 (m), 4.50-3.50 (m), 1.52-1.17 (br).

### 201m) 5-Bromo-pyridine-2-carboxylic acid [3-(6-amino-2-difluoromethyl-2,3,4,5-tetrahydro-pyrazin-2-yl)-4-fluoro-phenyl]-amide

A mixture of compound 201l (33 mg, 0.051 mmol) and 0.5 ml of 3N HCl in MeOH was stirred overnight. The mixture was evaporated, redissolved in methanol and triturated with TBME to give the hydrochloride salt of the title compound as white crystals. {TLC (DCM: MeOH: NH₃ (25%, aq)/90:10:0.5) Rf 0.27; HPLC: Rt_{H2}=2.677 min; ESIMS: 442, 444 [(M+H)⁺, 1Br]; ¹H-NMR (600 MHz, DMSO-d₆): 10.90 (s, 1H), 10.73 (br s, 1H), 9.79 (br s, 1H), 8.88 (s, 1H), 8.66 (br s, 1H), 8.35 (d, 1H), 8.14-8.00 (m, 3H), 7.41-7.33 (m, 1H), 6.85-6.61 (m, 1H), 3.95 (d, 1H), 3.87 (d, 1H), 3.56 (d, 1H), 3.48 (d, 1H)}.

### Example 202

### 5-Cyano-pyridine-2-carboxylic acid [3-(6-amino-2-difluoromethyl-2,3,4,5-tetrahydro-pyrazin-2-yl)-4-fluoro-phenyl]-amide

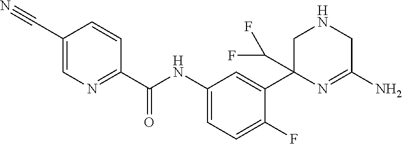 [SMILES: N#Cc1ccc(C(=O)Nc2ccc(F)c(C3(C(F)F)CNCC(N)=N3)c2)nc1]

The title compound was prepared by procedures analogous to those used in Example 201 hereinbefore. {TLC (DCM: MeOH: NH3 (25%, aq)/90:10:0.5) Rf 0.22; HPLC: Rt_{H1}=2.783 min; ESIMS: 389 [(M+H)⁺]; ¹H-NMR (360 MHz, DMSO-d₆): 10.78 (br s, 1H), 9.22 (s, 1H), 8.60 (dd, 1H), 8.30 (d, 1H), 8.10-8.05 (m, 1H), 7.88 (br s, 1H), 7.21 (br s, 1H), 6.15 (t, 1H, J=56 Hz), 5.92 (s, 1H), 3.31-2.98 (br m, 4H)}.

### Example 203

### 5-Cyano-pyridine-2-carboxylic acid [3-(6-amino-2-difluoromethyl-5-oxo-2,3,4,5-tetrahydro-pyrazin-2-yl)-4-fluoro-phenyl]-amide

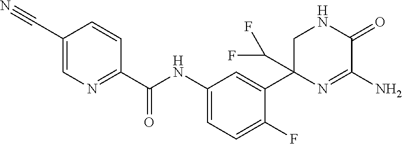 [SMILES: N#Cc1ccc(C(=O)Nc2ccc(F)c(C3(C(F)F)CNC(=O)C(N)=N3)c2)nc1]

The title compound was isolated as a side product from the preparation of Example 202. {TLC (DCM: MeOH: NH3 (25%, aq)/90:10:0.5) Rf 0.32; HPLC: Rt_{H1}=2.780 min; ESIMS: 403 [(M+H)⁺]; ¹H-NMR (360 MHz, DMSO-d₆): 10.83 (s, 1H), 9.14 (d, 1H), 8.52 (dd, 1H), 8.40 (s, 1H), 8.21 (d, 1H), 8.07 (dd, 1H), 7.84-7.77 (m, 1H), 7.18 (dd, 1H), 6.66 (br s, 1H), 6.14 (t, 1H, J=56 Hz), 3.81 (d, 1H), 3.70 (d, 1H}.

### Example 204

### 5-Amino-3-{5-[(5-bromo-pyridine-2-carbonyl)-amino]-2-fluoro-phenyl}-3-difluoromethyl-3,6-dihydro-2H-pyrazine-1-carboxylic acid methyl ester

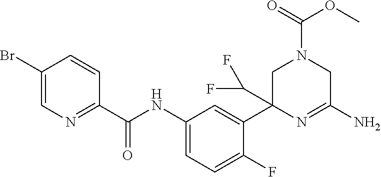 [SMILES: COC(=O)N1CC(N)=NC(c2cc(NC(=O)c3ccc(Br)cn3)ccc2F)(C(F)F)C1]

The title compound was prepared by procedures analogous to Example 201, except that in step 16g methyl chloroformate was used instead of Boc2O. {TLC (DCM: MeOH: NH3 (25%, aq)/90:10:0.5) Rf 0.35; HPLC: Rt_{H1}=3.120 min; ESIMS: 500, 502 [(M+H)⁺, 1Br]; ¹H-NMR (600 MHz, DMSO-d₆ 1:1.5 mixture of rotamers): 11.01 (d, 1H), 10.95 (d, 1H), 9.89 (br s, 1H), 8.88 (s, 1H), 8.79 (d, 1H), 8.35 (d, 1H), 8.09 (d, 1H), 7.94 (br s, 1H), 7.39 (br s, 1H), 6.81 (t, 1H, J=54 Hz), 4.67-4.50 (m, 2H), 4.33-4.22 (m, 1H), 3.94 (d, 1H), 3.56/3.40 (2 s, 3H)}.

### Example 205

### 5-Amino-3-{5-[(5-cyano-pyridine-2-carbonyl)-amino]-2-fluoro-phenyl}-3-difluoromethyl-3,6-dihydro-2H-pyrazine-1-carboxylic acid methyl ester

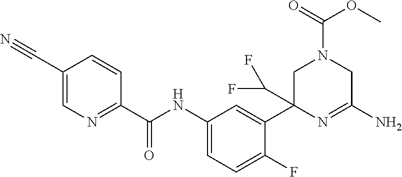 [SMILES: COC(=O)N1CC(N)=NC(c2cc(NC(=O)c3ccc(C#N)cn3)ccc2F)(C(F)F)C1]

The title compound was prepared by procedures analogous to those used in the examples hereinbefore {TLC (DCM: MeOH: NH3 (25%, aq)/90:10:0.5) Rf 0.60; HPLC: Rt_{H1}=2.946 min; ESIMS: 447 [(M+H)⁺]; ¹H-NMR (400 MHz, DMSO-d₆: 10.81 (s, 1H), 9.18 (s, 1H), 8.56 (dd, 1H), 8.25 (d, 1H), 8.00 (br s, 1H), 7.82 (br s, 1H), 7.19 (dd, 1H), 6.29 (br s, 1H), 6.15 (t, 1H, J=54 Hz), 3.97-3.67 (m, 4H), 3.53/3.46 (2 s, 3H, rotameres, relation 1:1)}.

### Example 206

### 5-Bromo-3-methyl-pyridine-2-carboxylic acid [3-(4-acetyl-6-amino-2-difluoromethyl-2,3,4,5-tetrahydro-pyrazin-2-yl)-4-fluoro-phenyl]-amide

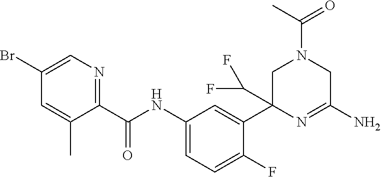 [SMILES: CC(=O)N1CC(N)=NC(c2cc(NC(=O)c3ncc(Br)cc3C)ccc2F)(C(F)F)C1]

### 206a) [1-Aminomethyl-2,2-difluoro-1-(2-fluoro-phenyl)-ethyl]-carbamic acid tert-butyl ester

Compound 201d (4.0 g, 10.44 mmol) and 1.713 g (20.88 mmol) NaOAc were suspended in 50 ml EtOH and stirred under a hydrogen atmosphere in the presence of 200 mg of Pd on carbon (5%), until all of the bromide was hydrogenated. The mixture was treated with 10% aq Na₂CO₃, filtered over celite, extracted with EtOAc, dried with Na₂SO₄ and evaporated. The residual oil was stirred with hexane and, after filtration, the title compound was isolated as a white solid. {HPLC: Rt_{H1}=2.908 min; ESIMS: 305 [(M+H)⁺]; 1H-NMR (360 MHz, CDCl3): 7.43 (t, 1H), 7.36 (q, 1H), 7.21 (t, 1H), 7.11 (dd, 1H), 6.48 (t, CHF2), 5.78 (br s, 1H), 3.52 (br d, 1H), 3.42 (br d, 1H), 1.43 (br s, 9H}.

### 206b) [1-[(Cyanomethyl-amino)-methyl]-2,2-difluoro-1-(2-fluoro-phenyl)-ethyl]-carbamic acid tert-butyl ester

A mixture of 14.0 g (46.0 mmol) compound 206a, 9.22 g (55.2 mmol) iodoacetonitrile and 17.84 g (138 mmol) DIPEA in 90 ml ACN was stirred at 80° C. for 3 h. The mixture was diluted with EtOAc, washed with 1N HCl, 5% aq NaHCO₃ and water. The organic phase was dried with sodium sulfate and the product was purified via chromatography on silica gel (heptane/30% EtOAc) to give the title compound as a yellowish oil. TLC: Rf 0.20 (EtOAc/heptane 1:3; HPLC: Rt_{H3}=2.682 min; ESIMS: 344 [(M+H)⁺]; ¹H-NMR (CDCl₃, 360 MHz): 7.44-7.34 (m, 2H), 7.22 (t, 1H), 7.13 (d d, 1H), 6.47 (t, 1H, CHF₂), 5.62 (br s, 1H), 3.72-3.37 (m, 4H), 1.95 (br s, 1H), 1.33 (s, 9H).

### 206c) [2-tert-Butoxycarbonylamino-3,3-difluoro-2-(2-fluoro-phenyl)-propyl]-cyanomethyl-carbamic acid 2,2,2-trichloro-ethyl ester

To a vigorously stirred suspension of 16.0 g (46.6 mmol) compound 206b in 80 ml DCM and 150 ml 10% aq NaHCO₃ were added dropwise 24.7 g (117 mmol) trichloroethyl chloroformate over a period of 10 minutes. The reaction temperature was kept below 26° C. with the aid of an ice-bath. Stirring was continued for 3.5 h at 25° C. The phases were separated and the organic phase was dried with MgSO₄.H₂O, filtered, evaporated and purified via chromatography on silica gel (heptane/15% EtOAc) to give the title compound as a colorless foam. TLC: Rf 0.50 (EtOAc/heptane 1:3; HPLC: Rt_{H6}=2.758 min; ESIMS: 518 [(M+H)⁺, 3Cl]; ¹H-NMR (CDCl₃, 360 MHz, broad signals, 2:1 mixture of rotamers): 7.45-7.35 (m, 2H), 7.30-7.22 (m, 1H), 7.15 (dd, 1H), 6.88-6.50 (m, 1H, CHF₂), 6.18 (br s, NH, major rotamer), 5.70 (br s, NH, minor rotamer), 4.92-4.22 (m, 6H), 1.48 (br s, 9H).

### 206d) 5-Amino-3-difluoromethyl-3-(2-fluoro-phenyl)-3,6-dihydro-2H-pyrazine-1-carboxylic acid 2,2,2-trichloro-ethyl ester

Compound 206c (24.17 g, 46.6 mmol) was dissolved in 93 ml DCM and treated with 87 ml 4N HCl in dioxane. After stirring for 4 h at room temperature the mixture was evaporated to yield the title compound as a colorless foam, pure enough for further syntheses.

TLC (DCM: MeOH: NH3 (25%, aq.)/90:10:0.5) Rf 0.42. Rt_{H1}=3.203 min; ESIMS: 418 [(M+H)⁺, 3Cl]; ¹H-NMR (DMSO-d6, 360 MHz, broad signals): 11.26 (s, 1H), 10.0-9.9 (br, 1H), 8.98 (br s, 1H), 7.65-7.50 (m, 2H), 7.48-7.35 (m, 2H), 6.83 (br t, CHF₂), 4.92-4.68 (m, 4H), 4.45-4.16 (m, 2H).

### 206e) 5-Amino-3-difluoromethyl-3-(2-fluoro-5-nitro-phenyl)-3,6-dihydro-2H-pyrazine-1-carboxylic acid 2,2,2-trichloro-ethyl ester

To a stirred solution of compound 206d (21.16 g, 46.5 mmol) in 60 ml 95% H₂SO₄ were added portion wise 6.11 g (60.5 mmol) KNO₃ while keeping the reaction temperature below 30° C. with the help of a water-bath. After 30 min the mixture was poured onto 200 g crushed ice and water. The mixture was neutralized with 4N NaOH and solid Na₂CO₃ (caution, foaming). The mixture was extracted with EtOAc twice, dried with Na₂SO₄ and evaporated the crude product was purified by crystallization from TBME/hexanes to give the title compound as a white solid. TLC: Rf 0.50 (EtOAc/heptane 1:3), Rt_{H1}=3.211 min; ESIMS: 463 [(M+H)⁺, 3Cl]; ¹H-NMR (DMSO-d6, 360 MHz, broad signals): 8.64-8.56 (m, 1H), 8.34-8.27 (m, 1H), 7.55 (t, 2H), 6.63 (br d, 2H), 6.22 (t, CHF₂), 4.90-4.72 (m, 2H), 4.23-3.85 (m, 4H).

### 206f) 5-tert-Butoxycarbonylamino-3-difluoromethyl-3-(2-fluoro-5-nitro-phenyl)-3,6-dihydro-2H-pyrazine-1-carboxylic acid 2,2,2-trichloro-ethyl ester

The title compound was prepared from compound 206e by a procedure similar to that used to obtain compound 201j.

TLC: Rf 0.36 (EtOAc/heptane 1:3), Rt_{H6}=3.010 min; ESIMS: 585 [(M+Na)⁺, 3Cl]; ¹H-NMR (DMSO-d6, 360 MHz, broad signals): 10.34 (br s, 1H), 8.70-8.64 (m, 1H), 8.37-8.30 (m, 1H), 7.59 (dd, 2H), 6.33 (br t, CHF₂), 4.93-4.66 (m, 3H), 4.53-4.28 (m, 2H), 3.84-3.75 (m, 1H).

### 206g) 3-(5-Amino-2-fluoro-phenyl)-5-tert-butoxycarbonylamino-3-difluoromethyl-3,6-dihydro-2H-pyrazine-1-carboxylic acid 2,2,2-trichloro-ethyl ester

A mixture of compound 206f (3 g, 5.32 mmol). 2.97 g (53.2 mmol) Fe and 3.42 g (63.9 mmol) NH₄Cl in 55 ml MeOH was refluxed for 3 h. The mixture was filtered over celite and washed with EtOAc. The organic phase was washed with 5% NaHCO₃, brine and was dried with Na₂SO₄ and purified via chromatography on silica gel (heptane/EtOAc 0-50% EtOAc) to give the title compound as a colorless foam.

TLC: Rf 0.32 (EtOAc/heptane 1:2), Rt_{H3}=2.842 min; ESIMS: 533 [(M+H)⁺, 3Cl]; ¹H-NMR (CDCl₃, 360 MHz, broad signals): 7.39 (br s, 1H), 6.98-6.84 (m, 2H), 6.75-6.55 (m, 3H), 6.28 (t, CHF₂), 4.90-3.55 (m, 6H), 1.55 and 1.52 (br s, 9H)

### 206h) 3-{5-[(5-Bromo-3-methyl-pyridine-2-carbonyl)-amino]-2-fluoro-phenyl}-5-tert-butoxycarbonylamino-3-difluoromethyl-3,6-dihydro-2H-pyrazine-1-carboxylic acid 2,2,2-trichloro-ethyl ester

The title compound was prepared from compound 206g by a procedure similar to that used to obtain compound 201g.

TLC: Rf 0.25 (EtOAc/heptane 1:3), {HPLC: Rt_{H6}=3.535 min; ESIMS: 730, 732 [(M+H)⁺, 1Br, 3Cl]; ¹H-NMR (360 MHz, CDCl₃): 10.18-9.98 (m, 1H), 8.54 (s, 1H), 8.11-7.98 (m, 1H), 7.85 (s, 1H), 7.60-7.45 (m, 2H), 7.13 (t, 1H), 6.78 (t, CHF2), 4.92-4.42 (m, 4H), 4.30-3.95 (m, 2H), 2.81 (s, 3H), 1.55 (s, 9H).

### 206i) (6-{5-[(5-Bromo-3-methyl-pyridine-2-carbonyl)-amino]-2-fluoro-phenyl}-6-difluoromethyl-3,4,5,6-tetrahydro-pyrazin-2-yl)-carbamic acid tert-butyl ester

A mixture of compound 206h (620 mg, 0.805 mmol), 526 mg (8.05 mmol) Zn powder and 43 mg (0.805 mmol) NH₄Cl in 4 ml MeOH was stirred 30 min. The mixture was made basic with a small amount 25% aq NH₄OH, filtered over celite and washed with MeOH and EtOAc. The filtrate was washed with brine, the aq phase extracted 3 times with EtOAc and the combined organic layers dried with Na₂SO₄. Purification via chromatography on silica gel (heptane/0-70% EtOAc/0.005% 25% aq NH4OH) gave the title compound as a colorless foam. TLC: Rf 0.31 (EtOAc/heptane 1:1), {HPLC: Rt_{H3}=2.864 min; ESIMS: 556, 558 [(M+H)⁺, 1Br]; ¹H-NMR (360 MHz, CDCl₃, broad signals due to rotamers): 10.15-10.0 (m, 1H), 8.54 (br s, 1H), 8.11-8.02 (m, 1H), 7.84 (s, 1H), 7.63-7.60 (m, 1H), 7.22-7.10 (m, 1H), 6.6-6.0 (br, CHF2), 4.10-3.2 (m, 4H), 2.81 (s, 3H), 1.56 and 1.51 (s, 9H).

### 206j) (4-Acetyl-6-{5-[(5-bromo-3-methyl-pyridine-2-carbonyl)-amino]-2-fluoro-phenyl}-6-difluoromethyl-3,4,5,6-tetrahydro-pyrazin-2-yl)-carbamic acid tert-butyl ester

A mixture of compound 206i (150 mg, 0.270 mmol), 55 mg (0.539 mmol) acetic anhydride and 45 mg (0.566 mmol) pyridine 1 ml DCM was stirred for 1 h. The mixture was quenched with 10% aq Na₂CO₃ and extracted with DCM. The org phase was dried with Na₂SO₄ and evaporated. Purification via chromatography on silica gel (heptane/EtOAc 0-50% EtOAc) gave the title compound as a colorless solid. TLC: Rf 0.19 (EtOAc/heptane 1:2), {HPLC: Rt_{H3}=3.242 min; ESIMS: 598, 600 [(M+H)⁺, 1 Br];

¹H-NMR (360 MHz, CDCl₃, ca 1:1 mixture of rotamers): 9.93 (br d, 1H), 8.44 (br s, 1H), 8.03-7.0 (m, 5H), 6.4-5.85 (m, 1H), 4.75-3.65 (m, 4H), 2.70 (s, 3H), 2.03 and 1.91 (s, 3H), 1.49 and 1.44 (s, 9H).

### 206k) 5-Bromo-3-methyl-pyridine-2-carboxylic acid [3-(4-acetyl-6-amino-2-difluoromethyl-2,3,4,5-tetrahydro-pyrazin-2-yl)-4-fluoro-phenyl]-amide

Compound 206j (140 mg, 0.234 mmol) was taken up in 0.5 ml DCM and 1 ml 4N HCl in dioxane was stirred 2 h. The mixture was evaporated, taken up in 10% Na₂CO₃ and EtOAc. The aq phase was extracted twice with EtOAc, the combined org layers were dried with Na₂SO₄. Purification via chromatography on silica gel (DCM/MeOH/25% aq NH4OH 90:10:0.5) gave the title compound as a colorless solid. {TLC (DCM: MeOH: NH3 (25%, aq)/90:10:0.5) Rf 0.34; HPLC: Rt_{H1}=3.071 min; ESIMS: 498, 500 [(M+H)⁺, 1Br]; ¹H-NMR (360 MHz, DMSO-d₆ (rotameric mixture, relation 2:1): 10.65-10.51 (m, 1H), 8.66 (s, 1H), 8.17 (s, 1H), 7.91-7.75 (m, 2H), 7.30-7.11 (m, 1H), 6.44-6.01 (m, 2H), 4.09-3.66 (m, 4H), 2.56-2.54 (m, 3H), 1.93-1.86 (m, 3H)}.

### Example 207

### 5-Amino-3-{5-[(5-bromo-3-methyl-pyridine-2-carbonyl)-amino]-2-fluoro-phenyl}-3-difluoromethyl-3,6-dihydro-2H-pyrazine-1-carboxylic acid 2,2-dichloro-ethyl ester

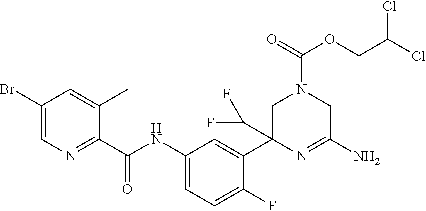 [SMILES: Cc1cc(Br)cnc1C(=O)Nc1ccc(F)c(C2(C(F)F)CN(C(=O)OCC(Cl)Cl)CC(N)=N2)c1]

The title compound was prepared from a side product isolated in step 206i. {TLC (DCM: MeOH: NH3 (25%, aq)/90:10:0.5) Rf 0.44; HPLC: Rt_{H3}=2.776 min; ESIMS: 598 [(M+H)⁺]; ¹H-NMR (400 MHz, DMSO-d₆, broad signals due to rotamers: 10.59-10.55 (m, 1H), 8.62 (d, 1H), 7.87-7.82 (m, 1H), 7.82-7.75 (m, 1H), 7.16 (br s, 1H), 6.37-6.28 (m, 3H), 4.41-4.34 (m, 2H), 3.97-3.71 (m, 4H), 2.52 (s, 3H)}.

### Example 208

### 5-Bromo-3-methyl-pyridine-2-carboxylic acid {3-[6-amino-2-difluoromethyl-4-(2-methoxy-acetyl)-2,3,4,5-tetrahydro-pyrazin-2-yl]-4-fluoro-phenyl}-amide

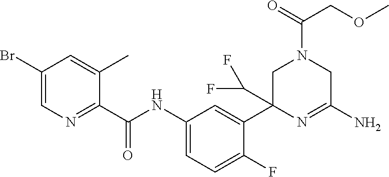 [SMILES: COCC(=O)N1CC(N)=NC(c2cc(NC(=O)c3ncc(Br)cc3C)ccc2F)(C(F)F)C1]

The title compound was prepared from compound 206i using methoxy-acetyl chloride instead of acetic anhydride and by procedures analogous to those used in Example 206. {TLC (DCM: MeOH: NH3 (25%, aq)/90:10:0.5) Rf 0.38; HPLC: Rt_{H1}=3.083 min; ESIMS: 528, 530 [(M+H)⁺, 1Br]; ¹H-NMR (360 MHz, DMSO-d₆(1:1 mixture of diastereomers): 10.64-10.52 (m, 1H), 8.66 (s, 1H), 8.16 (s, 1H), 7.90-7.76 (m, 2H), 7.29-7.13 (m, 1H), 6.47-6.04 (m, 2H), 6.41-6.28 (m, 2H), 6.22 (t, 1H, J=55 Hz), 4.08-3.74 (m, 6H), 3.22-3.15 (m, 3H), 2.55 (s, 3H)}.

### Example 209

### 5-Bromo-3-methyl-pyridine-2-carboxylic acid [3-(6-amino-4-cyclopropanecarbonyl-2-difluoromethyl-2,3,4,5-tetrahydro-pyrazin-2-yl)-4-fluoro-phenyl]-amide

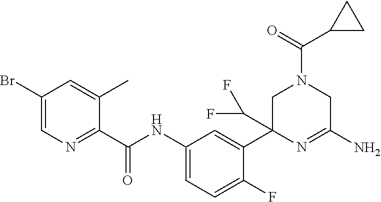 [SMILES: Cc1cc(Br)cnc1C(=O)Nc1ccc(F)c(C2(C(F)F)CN(C(=O)C3CC3)CC(N)=N2)c1]

The title compound was prepared from compound 206i using cyclopropanecarbonyl chloride instead of acetic anhydride and by procedures analogous to those used in Example 206. {HPLC: Rt_{H1}=3.187 min; ESIMS: 524, 526 [(M+H)⁺, 1Br]; ¹H-NMR (360 MHz, DMSO-d₆ (2:1 mixture of rotamers): 10.63-10.53 (m, 1H), 8.66 (s, 1H), 8.16 (s, 1H), 7.92-7.74 (m, 2H), 7.27-7.12 (m, 1H), 6.37-6.29 (m, 2H), 6.25 (t, 1H, J=55 Hz), 4.48-3.75 (m, 4H), 2.55 (s, 3H), 1.82-1.65 (m, 1H), 0.77-0.29 (m, 1H)}.

### Example 210

### 5-Bromo-3-methyl-pyridine-2-carboxylic acid [3-(6-amino-2-difluoromethyl-2,3,4,5-tetrahydro-pyrazin-2-yl)-4-fluoro-phenyl]-amide

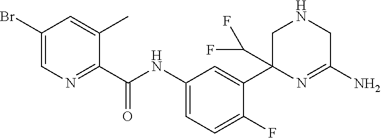 [SMILES: Cc1cc(Br)cnc1C(=O)Nc1ccc(F)c(C2(C(F)F)CNCC(N)=N2)c1]

The title compound was prepared from compound 206i via direct Boc-deprotection as in Example 206. {TLC (DCM: MeOH: NH3 (25%, aq)/90:10:0.5) Rf 0.20; HPLC: Rt_{H1}=3.050 min; ESIMS: 456, 458 [(M+H)⁺, 1Br]; ¹H-NMR (360 MHz, DMSO-d₆): 10.53 (s, 1H), 8.65 (s, 1H), 8.16 (s, 1H), 7.93-7.88 (m, 1H), 7.87-7.80 (m, 1H), 7.16 (dd, 1H), 6.13 (t, 1H, J=57 Hz), 5.90 (br s, 1H), 3.18 (t, 2H), 3.07 (t, 2H), 2.56 (s, 3H)}.

### Example 211

### 5-Difluoromethoxy-3-methyl-pyridine-2-carboxylic acid [3-(4-acetyl-6-amino-2-difluoromethyl-2,3,4,5-tetrahydro-pyrazin-2-yl)-4-fluoro-phenyl]-amide

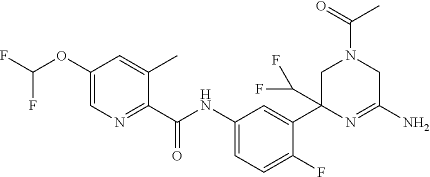 [SMILES: CC(=O)N1CC(N)=NC(c2cc(NC(=O)c3ncc(OC(F)F)cc3C)ccc2F)(C(F)F)C1]

The title compound was prepared from compound 206g by procedures analogous to those used in Example 206 and instead using Acid 5 as a coupling partner in the amide coupling. {HPLC: Rt_{H2}=2.795 min; ESIMS: 486 [(M+H)⁺]; ¹H-NMR (400 MHz, DMSO-d₆ (2:1 mixture of rotamers): 10.56-10.46 (m, 1H), 8.40 (s, 1H), 7.87-7.74 (m, 1H), 7.69 (s, 1H), 7.25-7.09 (m, 2H), 6.37-5.99 (m, 3H), 4.04-3.94 (m, 1H), 3.84 (s, 1H), 2.58-2.54 (m, 3H), 1.90-1.82 (m, 3H)}.

### Example 212

### 5-Difluoromethoxy-3-methyl-pyridine-2-carboxylic acid [3-((R)-6-amino-2-difluoromethyl-2,3,4,5-tetrahydro-pyrazin-2-yl)-4-fluoro-phenyl]-amide

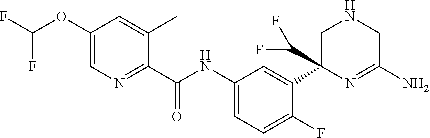 [SMILES: Cc1cc(OC(F)F)cnc1C(=O)Nc1ccc(F)c([C@]2(C(F)F)CNCC(N)=N2)c1]

The title compound was prepared from compound 206g by procedures analogous to those used in Example 206 and instead using Acid 5 as a coupling partner in the amide coupling. The enantiomers were separated on Chiralpak® OD-H, 30×250 mm column using CO₂/(MeOH+1% IPAm)/60:40 (isocratic) as an eluent. The title compound is the faster moving enantiomer. {HPLC: Rt_{H1}=3.047 min; ESIMS: 444 [(M+H)⁺]; 1H-NMR (400 MHz, DMSO-d₆): 10.46 (s, 1H), 8.40 (d, 1H), 7.91-7.86 (m, 1H), 7.84-7.78 (m, 1H), 7.42 (t, 1H, J=73 Hz), 7.12 (dd, 1H), 6.71 (t, 1H, J=56 Hz), 5.88 (br s, 2H), 3.20-2.98 (m, 4H), 2.57 (s, 3H)}.

### Example 213

### 3-Amino-5-methoxy-pyrazine-2-carboxylic acid [3-((R)-6-amino-2-difluoromethyl-2,3,4,5-tetrahydro-pyrazin-2-yl)-4-fluoro-phenyl]-amide

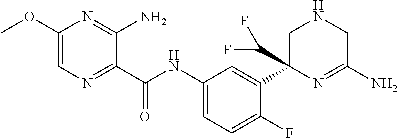 [SMILES: COc1cnc(C(=O)Nc2ccc(F)c([C@]3(C(F)F)CNCC(N)=N3)c2)c(N)n1]

The title compound was prepared from compound 206g by procedures analogous to those used in Example 206 and instead using Acid 12 as a coupling partner in the amide coupling. The enantiomers were separated on a Chiralpak® AD 20 um (5×50 cm) column using MeOH/EtOH/+0.01% DEA as an eluent. The title compound is the slower moving enantiomer. {HPLC: Rt_{H1}=2.889 min; ESIMS: 410 [(M+H)^{+];} 1H-NMR (400 MHz, DMSO-d₆): 10.04 (s, 1H), 7.88 (dd, 1H), 8.16 (s, 1H), 7.77-7.71 (m, 1H), 7.50 (s, 1H), 7.09 (dd, 1H), 6.09 (t, 1H, J=55 Hz), 5.88 (br s, 1H), 3.88 (s, 3H), 3.21-2.94 (m, 5H)}.

### Example 214

### 3-Amino-5-oxo-4,5-dihydro-pyrazine-2-carboxylic acid [3-((R)-6-amino-2-difluoromethyl-2,3,4,5-tetrahydro-pyrazin-2-yl)-4-fluoro-phenyl]-amide

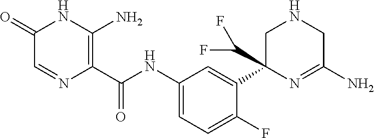 [SMILES: NC1=N[C@@](c2cc(NC(=O)c3ncc(=O)[nH]c3N)ccc2F)(C(F)F)CNC1]

The title compound was isolated during the purification of Example 213 as a side product. {HPLC: Rt_{H1}=2.427/2.547 min; ESIMS: 396 [(M+H)⁺]; 1H-NMR (600 MHz, DMSO-d₆): 10.00 (s, 1H), 9.90 (s, 1H), 8.90 (s, 1H), 8.02-7.97 (m, 1H), 7.84-7.80 (m, 1H), 7.30 (dd, 1H), 7.17 (s, 1H), 6.71 (t, 1H, J=54 Hz), 4.23 (d, 1H), 4.12 (d, 1H), 3.80 (s, 2H)}.

### Example 215

### 5-Chloro-3-methoxymethyl-pyridine-2-carboxylic acid [3-((R)-5-amino-3-difluoromethyl-3,6-dihydro-2H-[1,4]oxazin-3-yl)-4-fluoro-phenyl]-amide

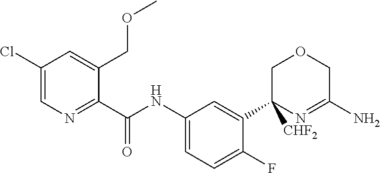 [SMILES: COCc1cc(Cl)cnc1C(=O)Nc1ccc(F)c([C@]2(C(F)F)COCC(N)=N2)c1]

### a) 1-(5-Bromo-2-fluoro-phenyl)-ethanone

A solution of diisopropyl amine (17.78 ml, 126 mmol) in THF (375 ml) was cooled to −78° C. A 1.6 M solution of BuLi in hexanes (79 ml, 126 mmol) was added drop wise. After 15 minutes 4-bromo-1-fluoro benzene (20 g, 114 mmol) was added dropwise while keeping the temperature below −60° C. After stirring for 2.5 h at −70° C. ethyl difluoro acetate (13.22 ml) were added. The mixture was warmed to −40° C. and then quenched by pouring the mixture onto 1M HCl. The mixture was extracted with ligroine, dried with MgSO₄.H₂O, concentrated and purified by column chromatography (silica gel; hexane/5-15% TBME) to give the desired product as a yellow liquid.

¹H-NMR (CDCl₃, 360 MHz): δ 8.09 (dd, 1H), 7.82-7.77 (m, 1H), 7.17 (t, 1H), 6.45 (t, 1H, CHF₂).

### b) 1-(5-Bromo-2-fluoro-phenyl)-1-difluoromethyl-allyl]-carbamic acid tert-butyl ester

A mixture of 1-(5-bromo-2-fluoro-phenyl)-ethanone (16 g, 63.2 mmol) and N-tert-butyloxycarbonyl-triphenyliminophosphorane (26.3 g, 69.6 mmol) were heated at 90° C. in toluene for 18 h. The mixture was triturated with hexane and filtered to remove triphenyl phosphine oxide. The filtrate was purified by chromatography on silica gel (hexane/1-5% TBME) to give 11.37 g (32.3 mmol) of the desired product as a slightly impure yellow oil. TLC: Rf (Hexane/EtOAc 6:1)=0.65.

The product was dissolved in THF (100 ml) and cooled to −78° C. Vinylmagnesium bromide (48 ml of a 1M solution in THF) was added dropwise, while the reaction temperature was not allowed to exceed −60° C. The mixture was stirred at −70° C. for 1 h before it was allowed to warm to 0° C. The reaction was quenched with 10% aq. ammonium chloride and extracted with TBME. The organic layer was washed with brine, treated with activated charcoal and MgSO₄.H₂O and filtered over celite. The filtrated was concentrated and crystallized from hexane to give the desired product as colorless crystals.

HPLC: Rt_{H1}=3.575 min; ESIMS [M+Na]⁺=402/404(1Br);

¹H-NMR (CDCl₃, 360 MHz): δ 7.57 (dd, 1H), 7.51-7.45 (m, 1H), 7.00 (dd, 1H), 6.49 (t, 1H, CHF₂), 6.21 (dd, 1H), 5.59 (d, 1H), 5.40 (dd, 1H), 5.25 (br, 1H), 1.40 (br s, 9H).

### c) [1-(5-Bromo-2-fluoro-phenyl)-2,2-difluoro-1-hydroxymethyl-ethyl]-carbamic acid tert-butyl ester

A suspension of 1-(5-bromo-2-fluoro-phenyl)-1-difluoromethyl-allyl]-carbamic acid tert-butyl ester (10.99 g, 28.9 mmol) and sodium hydrogen carbonate (3.84 g, 43.4 mmol) in DCM (200 ml) and MeOH (80 ml) was cooled to −78° C. A mixture of O₃ in oxygen gas was introduced till the blue color persisted. The excess ozone was removed by bubbling through oxygen gas for 10 minutes. NaBH₄ (2.187 g, 57.8 mmol) was added as a solid in three portions. The mixture was stirred 10 min at −78° C. and then allowed to warm to 0° C. After 30 min the mixture was poured onto ice-cold 1N HCl and extracted with TBME. The organic phase was washed with 1N HCl, brine, dried with MgSO₄.H₂O and evaporated. The crude product was crystallized from hexane to give the desired product as colorless crystals.

TLC: Rf (Hexane/EtOAc 4:1)=0.29;

HPLC: Rt_{H1}=3.000 min; ESIMS [M+Na]⁺=406/408(1Br);

¹H-NMR (DMSO-d6, 360 MHz): δ 7.60-7.49 (m, 2H), 7.42 (br s, 1H), 7.180 (dd, 1H), 6.49 (t, 1H, CHF₂), 5.27 (br s, 1H), 3.90 (br s, 2H), 1.35 (br s, 9H).

### d) N-[1-(5-Bromo-2-fluoro-phenyl)-2,2-difluoro-1-hydroxymethyl-ethyl]-2-chloro-acetamide

A suspension of [1-(5-bromo-2-fluoro-phenyl)-2,2-difluoro-1-hydroxymethyl-ethyl]-carbamic acid tert-butyl ester (10.22 g, 26.6 mmol) in 4N HCl in dioxane (133 ml) was stirred for two h at rt. The mixture was evaporated to give the hydrochloride salt of 2-amino-2-(5-bromo-2-fluoro-phenyl)-3,3-difluoro-propan-1-ol.

HPLC: Rt_{H3}=2.550 min; ESIMS [M+H]⁺=284,286(1Br)

The crude product was taken up in DCM (63 ml) and 10% aq. soda (63 ml) and stirred vigorously with ice-cooling. A solution of chloroacetyl chloride (3.34 ml, 42 mmol) in DCM (10 ml) was added dropwise. The ice bath was taken away and stirring was continued for 1 h. The mixture was diluted with TBME and water. The organic phase was dried with MgSO₄.H₂O and purified via chromatography on silica gel (hexane/25-33% EtOAc) to give the desired product as a slightly impure resin.

HPLC: Rt_{H3}=3.336 min; ESIMS [M+H]⁺=360/362/364 (1Br, 1Cl);

¹H-NMR (DMSO-d6, 360 MHz): δ 8.78 (s, 1H), 7.62-7.53 (m, 2H), 7.19 (dd, 1H), 6.53 (t, 1H, CHF₂), 5.43 (t, 1H), 4.27-4.02 (m, 4H).

### e) 5-(5-Bromo-2-fluoro-phenyl)-5-difluoromethyl-morpholin-3-one

A solution of N-[1-(5-bromo-2-fluoro-phenyl)-2,2-difluoro-1-hydroxymethyl-ethyl]-2-chloro-acetamide (9.59 g, 26.2 mmol) in t-butanol (134 ml) was treated with KOtBu (3.58 g). The mixture was heated at reflux for 3 h. After cooling down the mixture was diluted with EtOAc and 1N HCl. The organic phase was washed with brine, dried with MgSO₄.H₂O, filtered and evaporated. The product was obtained as colorless crystal (TBME/hexane).

TLC: Rf (Hexane/EtOAc 2:1)=0.29;

HPLC: Rt_{H3}=2.950 min; ESIMS [M+H]⁺=324/326(1Br);

¹H-NMR (CDCl3, 360 MHz): δ 7.61-7.55 (m, 2H), 7.09 (dd, 1H), 6.80 (br, 1H), 6.35 (t, 1H, CHF2), 4.37-4.17 (m, 4H).

### f) 5-Difluoromethyl-5-(2-fluoro-phenyl)-morpholin-3-one

5-(5-Bromo-2-fluoro-phenyl)-5-difluoromethyl-morpholin-3-one (190 g, 586 mmol) and sodium acetate (57.7 g, 703 mmol) were suspended in 1850 mL methanol. Eventually, 10% Pd on charcoal (18.7 g) were added and the rm was shaked in a Parr apparatus in an atmosphere of hydrogen at rt. After 60 minutes the reaction mixture was filtered over celite and evaporated. The residue was dissolved in 2 l TBME, washed with aq NaHCO₃ and brine. The organic layer was dried over MgSO₄.H₂O and evaporated to give 143.2 g of the title compound as a white solid.

HPLC: Rt_{H1}=0.792 min; ESIMS [M+H]⁺=246;

¹H-NMR (CDCl₃, 360 MHz): δ 7.50-7.43 (m, 2H), 7.32-7.27 (m, 1H), 7.19 (dd, 1H), 6.62 (br, 1H), 6.37 (t, J=54 Hz, 1H), 4.34 (d, 1H), 4.31 (d, 1H), 4.22 (d, 1H), 4.20 (d, 1H).

### g) 5-Difluoromethyl-5-(2-fluoro-phenyl)-morpholine-3-thione

A mixture of 5-difluoromethyl-5-(2-fluoro-phenyl)-morpholin-3-one (141 g, 575 mmol) and Lawesson's reagent (132 g, 316 mmol) in 1400 ml of THF was heated at 68° C. for 1 h, cooled down and then evaporated. The residue was dissolved in 1 l DCM and filtered over 2 kg silica gel with 10 l DCM to give 161 g of the title compound in the form of a greenish resin that slowly crystallized. The compound was used without further purification.

HPLC: Rt_{H1}=1.799 min; ESIMS [M+H]⁺=262;

¹H-NMR (360 MHz, CDCl₃): δ 7.42-7.35 (m, 1H), 7.28 (t, 1H), 7.19 (t, 1H), 7.11 (dd, 1H), 6.29 (t, J=54 Hz, 1H), 4.57 (d, 1H), 4.47 (d, 1H), 4.21 (d, 1H), 4.18 (d, 1H).

### h) 5-Difluoromethyl-5-(2-fluoro-phenyl)-5,6-dihydro-2H-[1,4]oxazin-3-ylamine

5-Difluoromethyl-5-(2-fluoro-phenyl)-morpholine-3-thione (160 g, 570 mmol) was dissolved in 2.4 l of a NH₃ solution 7 mol/l in methanol for 6.5 h and afterwards left standing overnight. The reaction mixture was evaporated and taken up in 2 l 1N aq HCl and 2 l TBME. The aq phase was washed with TBME and made basic through the addition of 30% aq. NaOH (300 ml) and some ice. The mixture was extracted with DCM three times and the combined organic layers were dried with Na₂SO₄ and concentrated in vacuo. The title compound was obtained by crystallization from DCM/heptanes (128.45 g).

HPLC: Rt_{H3}=2.059 min; ESIMS [M+H]⁺=245;

¹H-NMR (CDCl₃, 360 MHz): δ 7.77 (t, 1H), 7.38-7.30 (m, 1H), 7.21 (t, 1H), 7.09 (dd, 1H), 6.19 (t, J=54 Hz, 1H), 4.51 (br, 2H), 4.32, (d, 1H), 4.18 (d, 1H), 4.05 (d, 1H), 3.96 (d, 1H), 1.39 (s, 3H), 1.24 (s, 3H).

### i) 5-Difluoromethyl-5-(2-fluoro-5-nitro-phenyl)-5,6-dihydro-2H-[1,4]oxazin-3-ylamine

Potassium nitrate (60.3 g, 596 mmol) was added portionwise to 600 ml sulfuric acid (T<20° C.). This solution was added dropwise to a solution of 5-difluoromethyl-5-(2-fluoro-phenyl)-5,6-dihydro-2H-[1,4]oxazin-3-ylamine (112 g, 459 mmol) in sulfuric acid (600 ml), while keeping the reaction temperature <22° C. with an ice bath. After stirring for 1 h, the mixture was poured onto 10 kg ice. TBME (6 l) was added and the pH was adjusted to 12-14 by the addition of 30% aq NaOH (ca. 5 l). The phases were separated and the aq. phase was extracted twice with TBME. The compined org layers were dried with sodium sulfate and evaporated to give 130 g of a yellow solid that was used further without purification.

HPLC: Rt_{H3}=2.063 min; ESIMS [M+H]⁺=290;

¹H-NMR (CDCl₃, 360 MHz): δ 8.71 (dd, 1H), 8.13 (dt, 1H), 7.13 (dd, 1H), 5.99 (t, J=54 Hz, 1H), 4.55 (br, 2H), 4.33 (dd, 1H), 4.10 (d, 1H), 3.97 (d, 1H), 3.82 (dt, 1H).

### j) [5-Difluoromethyl-5-(2-fluoro-5-nitro-phenyl)-5,6-dihydro-2H-[1,4]oxazin-3-yl]-carbamic acid tert-butyl ester

A solution of 5-Difluoromethyl-5-(2-fluoro-5-nitro-phenyl)-5,6-dihydro-2H-[1,4]oxazin-3-ylamine (144.5 g, 500 mmol), Boc anhydride (142 g, 650 mmol) and DIPEA (131 ml, 749 mmol) in 2500 ml THF was stirred for 3 days at rt. There was still tarting material left. Boc anhydride (56 g, 325 mmol) was added, the mixture was heated to 60° C. and stirred for 10 h till the reaction was complete. The mixture was evaporated, dissolved in TBME, washed with ice-cold 1N aq HCl, water, 10% aq. NaHCO₃ and brine. The org phase was dried with sodium sulfate, filtered and evaporated. The product was purified by crystallization from DCM/heptanes. Yield 182.8 g white crystals.

HPLC: Rt_{H1}=3.259 min; ESIMS [M+Na]⁺=412;

¹H-NMR (CDCl₃, 360 MHz): δ 8.70 (dd, 1H), 8.27 (dt, 1H), 7.34 (br, 1H), 7.25 (dd, 1H), 6.09 (t, J=54 Hz, 1H), 4.85 (d, 1H), 4.58 (d, 1H), 4.49 (dd, 1H), 3.94 (dt, 1H).

### k) [5-(5-Amino-2-fluoro-phenyl)-5-difluoromethyl-5,6-dihydro-2H-[1,4]oxazin-3-yl]-carbamic acid tert-butyl ester

[5-Difluoromethyl-5-(2-fluoro-5-nitro-phenyl)-5,6-dihydro-2H-[1,4]oxazin-3-yl]-carbamic acid tert-butyl ester (180 g, 462 mmol) and 17.61 g Pd—C 10% were suspended in THF (1760 ml). The mixture was shaked in a Parr apparatus in an atmosphere of hydrogen at rt. After 6 h the rm was filtered over celite and evaporated. The residue was crystallized from DCM/heptanes to provide 157.6 g of the title compound as beige crystals.

HPLC: Rt_{H3}=2.748 min; ESIMS [M+H]⁺=360;

¹H-NMR (CDCl₃, 360 MHz): δ Spectrum uninterpretable due to the presence of a complex mixture of rotamers.

### l) [(R)-5-(5-Amino-2-fluoro-phenyl)-5-difluoromethyl-5,6-dihydro-2H-[1,4]oxazin-3-yl]-carbamic acid tert-butyl ester

The racemic product ((rac.)[5-(5-Amino-2-fluoro-phenyl)-5-difluoromethyl-5,6-dihydro-2H-[1,4]oxazin-3-yl]-carbamic acid tert-butyl ester) was separated via prep. HPLC on a Chiralpak AD-H 20 μm (8×100×48 mm HPLC columns), on a Bayer SMB CC50 instrument using SMB technology with heptane/EtOH/MeOH 70:20:10 as eluent. The desired compound was the slower eluting (R)-enantiomer. Yield 72.29 g of the title compound as a colorless foam. ee=99.3%; Opt. rotation: [α]ₚ-97.5° (c=1, CHCl₃)

HPLC: Rt_{H3}=2.748 min; ESIMS [M+H]⁺=360;

¹H-NMR (CDCl₃, 360 MHz): δ Spectrum uninterpretable due to the presence of a complex mixture of rotamers.

### m) ((R)-5-{5-[(5-Chloro-3-methoxymethyl-pyridine-2-carbonyl)-amino]-2-fluoro-phenyl}-5-difluoromethyl-5,6-dihydro-2H-[1,4]oxazin-3-yl)-carbamic acid tert-butyl ester

5-Chloro-3-methoxymethyl-pyridine-2-carboxylic acid (56 mg, 0.278 mmol), [(R)-5-(5-Amino-2-fluoro-phenyl)-5-difluoromethyl-5,6-dihydro-2H-[1,4]oxazin-3-yl]-carbamic acid tert-butyl ester (100 mg, 0.278 mmol) and HOAt (68.2 mg, 0.50 mmol) were suspended in DMF (20 ml) and cooled down to 0° C. DIPEA (0.146 ml, 0.835 mmol) and EDC (80 mg, 0.417 mmol) were added and the reaction mixture was stirred at room temperature for 20 h. The reaction mixture was diluted with ethyl acetate, washed with water and brine, dried over sodium sulfate, filtered and evaporated. The crude product (592 mg) was chromatographed over silica gel (cyclohexane/ethyl acetate) to provide the title compound as a white glassy solid. TLC Rf (5:1 cyclohexane:ethyl acetate)=0.31;

MS: ESI+ 543, 545); ¹H-NMR (360 MHz, CDCl₃): δ 10.03 (s, br. 1H), 8.45 (m, 1H), 8.21 (m, 1H), 8.01 (m, 1H), 7.66 (m, 1H), 7.09 (m, 1H), 6.14 (t, 1H, CHF2), 5.09 (s, 2H), 4.79 (d, 1H), 4.56 (d, 1H), 4.38 (d, 1H), 3.95 (d, 1H), 3.55 (s, 3H), 1.49 (s, 9H).

### n) 5-Chloro-3-methoxymethyl-pyridine-2-carboxylic acid [3-((R)-5-amino-3-difluoromethyl-3,6-dihydro-2H-[1,4]oxazin-3-yl)-4-fluoro-phenyl]-amide

To a solution of ((R)-5-{5-[(5-Chloro-3-methoxymethyl-pyridine-2-carbonyl)-amino]-2-fluoro-phenyl}-5-difluoromethyl-5,6-dihydro-2H-[1,4]oxazin-3-yl)-carbamic acid tert-butyl ester (150 mg, 0.276 mmol) in dichloromethane (4 ml) was added TFA (0.35 ml, 4.54 mmol) and the reaction mixture was stirred for 18 h at room temperature. The solvent was removed in vacuo and the residue diluted with ethyl acetate and poured onto a mixture of ammonia 2N/ice. The layers were separated and the organic phase was washed with water and brine, dried over sodium sulfate, filtered and evaporated. 116 mg. Silica gel chromatography (dichloromethane/methanol 95:5+1% ammonia) afforded the title compound. 102 mg.

TLC R_{f}=0.48 (dichloromethane/methanol 95:5+1% ammonia); ESI+MS 443, 445;

HPLC-MS: Rt_{H12}=1.87 min. (99% purity, ESI+443, 445);

¹H-NMR (600 MHz, DMSO-D₆): δ 10.65 (s, 1H), 8.68 (s, 1H), 8.10 (s, 1H), 8.02 (m, 1H), 7.80 (m, 1H), 7.18 (m, 1H), 6.17 (m, 3H, CHF2, NH2 (amidine)), 4.88 (s, 2H), 4.14 (d, 1H), 4.02 (d, 1H), 3.95 (d, 1H), 3.88 (d, 1H), 3.41 (s, 3H).

### Examples 216 to 227

The compounds listed in Table 22 were prepared by a procedure analogous to those used in Example 215.

Hydrochloride salts were obtained from solutions of the corresponding free base by addition of hydrochloric acid in dioxane or hydrochloric acid in diethylether and evaporation of the solvents.

TABLE 22

| | | | MS |
|---|---|---|---|
| | | | [m/z; |
| Example | Compound | ¹H-NMR (δ; DMSO-d₆) | (M + 1)⁺] |
| 216 | 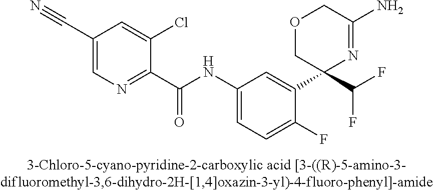 [SMILES: N#Cc1cnc(C(=O)Nc2ccc(F)c([C@]3(C(F)F)COCC(N)=N3)c2)c(Cl)c1] | 10.92 (s, 1H, NH), 9.10 (s, 1H), 8.80 (s, 1H), 7.91 (m, 1H), 7.80 (m, 1H), 7.20 (triplettoid, 1H), 6.17 (broad, 2H, NH₂ (amidine)), 6.17 (t, 1H, CHF2), 4.11 (d, 1H), 4.01 (d, 1H), 3.91 (d, 1H), 3.82 (d, 1H). | 424, 426 |
| 217 | 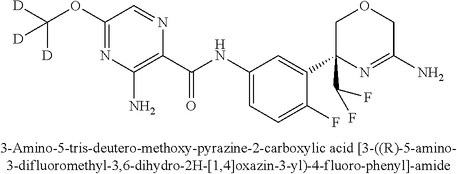 [SMILES: [2H]C([2H])([2H])Oc1cnc(C(=O)Nc2ccc(F)c([C@]3(C(F)F)COCC(N)=N3)c2)c(N)n1] | 10.10 (s, 1H), 8.01 (dd, 1H), 7.80-7.67 (m, 1H), 7.50 (s, 1H), 7.11 (dd, 1H), 6.12 (br. t, 2H, CHF2 + 1H), 4.13 (dd, 1H), 4.04-3.95 (m, 1H), 3.93-3.85 (m, 1H), 3.79 (d, 1H). | 414 |
| 218 | 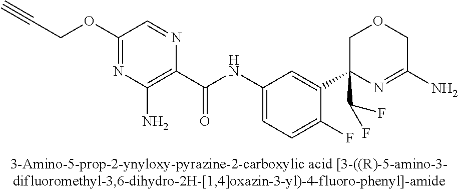 [SMILES: C#CCOc1cnc(C(=O)Nc2ccc(F)c([C@]3(C(F)F)COCC(N)=N3)c2)c(N)n1] | 10.17 (s, 1H), 8.05 (dd, 1H), 7.82-7.69 (m, 2H), 7.57 (s, 1H), 7.14 (dd, 1H), 6.14 (br. t, 2H, CHF2 + 1H), 5.02 (d, 2H), 4.15 (dd, 1H), 4.07-3.97 (m, 1H), 3.97-3.87 (m, 1H), 3.82 (d, 1H), 3.62 (t, 1H). | 435 |
| 219 | 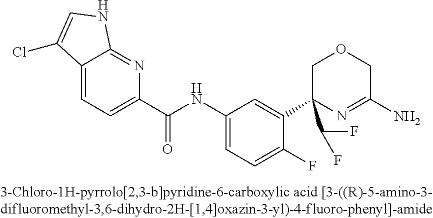 [SMILES: NC1=N[C@@](c2cc(NC(=O)c3ccc4c(Cl)c[nH]c4n3)ccc2F)(C(F)F)COC1] | 12.30 (s, 1H), 10.35 (s, 1H), 8.18 (d, 1H), 8.03 (broad, 1H), 7.98 (m, 2H), 7.90 (broad, 1H), 7.21 (triplettoid, 1H), 6.20 (broad, 2H, NH2), 6.18 (t, 1H, CHF2), 4.12 (d, 1H), 4.04 (d, 1H), 3.95 (d, 1H), 3.87 (d, 1H). | 438, 440 |
| 220 | 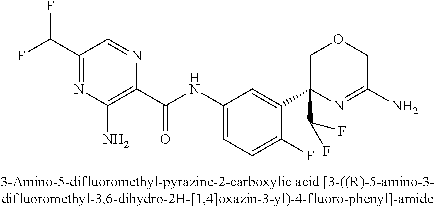 [SMILES: NC1=N[C@@](c2cc(NC(=O)c3ncc(C(F)F)nc3N)ccc2F)(C(F)F)COC1] | 10.66 (s, 1H), 8.14 (s, 1H), 8.10 (d, 1H), 7.91 (br. s, 2H), 7.76 (br. s, 1H), 7.17 (t, 1H), 6.96 (t, 1H, CHF2), 6.23-6.04 (m, 3H, CHF2 + 1H), 4.14 (d, 1H), 4.05-3.97 (m, 1H), 3.96-3.87 (m, 1H), 3.82 (d, 1H). | 431 |
| 221 | 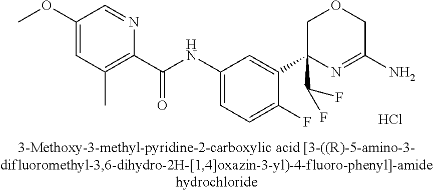 [SMILES: COc1cnc(C(=O)Nc2ccc(F)c([C@]3(C(F)F)COCC(N)=N3)c2)c(C)c1 ; Cl] | 10.99 (s, 1H), 10.63 (s, 1H), 9.76 (br s, 1H), 8.76 (s, 1H), 8.23 (d, 1H), 8.05-8.00 (m, 1H), 7.97 (dd, 1H), 7.44 (d, 1H), 7.35 (dd, 1H), 6.78 (t, 1H), 4.71 (d, 1H), 4.64 (d, 1H), 4.35 (d, 1H), 4.17 (d, 1H), 3.91 (d, 3H), 2.63 (s, 3H). | 409 |
| 222 | 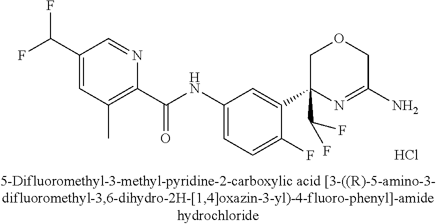 [SMILES: Cc1cc(C(F)F)cnc1C(=O)Nc1ccc(F)c([C@]2(C(F)F)COCC(N)=N2)c1 ; Cl] | 11.04 (br s, 1H), 10.87 (s, 1H), 9.78 (br s, 1H), 8.80 (s, 1H), 8.74 (s, 1H), 8.08 (s, 1H), 8.02-7.96 (m, 2H), 7.38 (dd, 1H), 7.25 (t, 1H), 6.79 (t, 1H), 4.72 (d, 1H), 4.65 (d, 1H), 4.35 (d, 1H), 4.18 (d, 1H), 2.61 (s, 3H). | 429 |
| 223 | 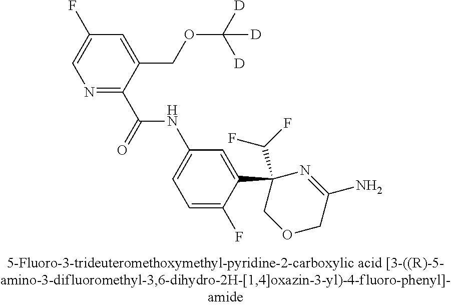 [SMILES: [2H]C([2H])([2H])OCc1cc(F)cnc1C(=O)Nc1ccc(F)c([C@]2(C(F)F)COCC(N)=N2)c1] | 10.59 (s, 1H, NH), 8.62 (d, 1H), 8.01 (m, 1H), 7.88 (dd, 1H), 7.78 (broad, 1H), 7.16 (triplettoid, 1H), 6.15 (broad, 2H, NH2), 6.12 (t, 1H, CHF2), 4.85 (s, 2H), 4.11 (d, 1H), 4.00 (d, 1H), 3.90 (d, 1H), 3.83 (d, 1H). | 430 |
| 224 | 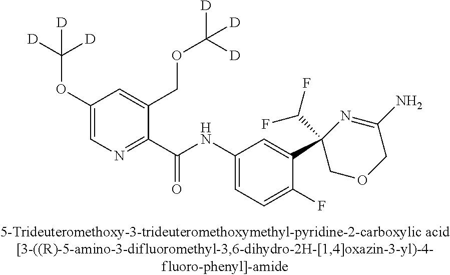 [SMILES: [2H]C([2H])([2H])OCc1cc(OC([2H])([2H])[2H])cnc1C(=O)Nc1ccc(F)c([C@]2(C(F)F)COCC(N)=N2)c1] | 11.0 (s, 1H, NH), 10.69 (s, 1H, NH, amide), 9.63 (s, 1H, NH₂, amidine), 8.76 (s, 1H, NH₂, amidine), 8.29 (d, 1H), 8.02 (m, 1H), 7.96 (dd, 1H), 7.60 (d, 1H), 7.33 (dd, 1H), 6.76 (t, 1H, CHF₂), 4.90 (s, 2H), 4.65 (d, 1H, AB), 4.61 (d, 1H, AB), 4.32 (d, 1H, AB), 4.14 (d, 1H, AB). | 445 |
| 225 | 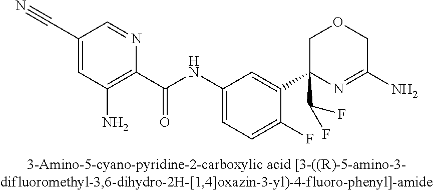 [SMILES: N#Cc1cnc(C(=O)Nc2ccc(F)c([C@]3(C(F)F)COCC(N)=N3)c2)c(N)c1] | 10.53 (s, 1H), 8.21 (d, 1H), 8.05 (dd, 1H), 7.77 (d, 1H), 7.64 (d, 1H), 7.25 (br. s, 2H), 7.16 (dd, 1H), 6.16 (br. s, 2H), 6.13 (t, 1H, CHF2), 4.14 (d, 1H), 4.01 (d, 1H), 3.91 (d, 1H), 3.81 (d, 1H). | 405 |
| 226 | 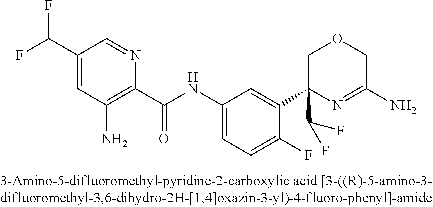 [SMILES: NC1=N[C@@](c2cc(NC(=O)c3ncc(C(F)F)cc3N)ccc2F)(C(F)F)COC1] | 10.47 (s, 1H), 8.08-8.03 (m, 1H), 8.02 (s, 1H), 7.79 (d, 1H), 7.41 (s, 1H), 7.18-7.13 (m, 3H), 7.12 (t, 1H, CHF2), 6.17 (br. s, 2H), 6.13 (t, 1H, CHF2), 4.22-4.11 (m, 1H), 4.07-3.97 (m, 1H), 3.91 (d, 1H), 3.81 (d, 1H). | 430 |
| 227 | 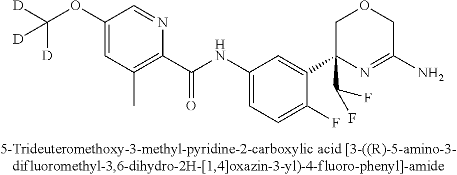 [SMILES: [2H]C([2H])([2H])Oc1cnc(C(=O)Nc2ccc(F)c([C@]3(C(F)F)COCC(N)=N3)c2)c(C)c1] | 10.38 (s, 1H), 8.22 (d, 1H), 7.99 (dd, 1H), 7.85-7.80 (m, 1H), 7.40 (dd, 1H), 7.15 (dd, 1H), 6.29-5.98 (m, 3H), 4.13 (dd, 1H), 4.02 (d, 1H), 3.92 (d, 1H), 3.83 (d, 1H), 2.62 (s, 3H). | 412 |

### Example 228

### 5-Cyano-3-methyl-pyridine-2-carboxylic acid [3-((R)-5-amino-3-difluoromethyl-3,6-dihydro-2H-[1,4]oxazin-3-yl)-4,5-difluoro-phenyl]-amide

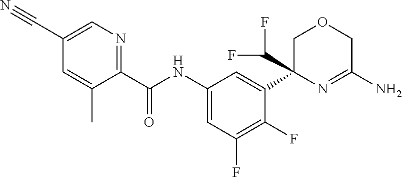 [SMILES: Cc1cc(C#N)cnc1C(=O)Nc1cc(F)c(F)c([C@]2(C(F)F)COCC(N)=N2)c1]

### a) 1-(2,3-Difluoro-phenyl)-2,2-difluoro-ethanone

A solution of 1,2-difluorobenzene (49.74 g, 436 mmol) in 700 ml THF was cooled to −70° C. Buli (1.6 M solution in hexanes, 272 ml, 436 mmol) was added dropwise while maintaining a reaction temperature <−60° C. After stirring for 2.5 h at −70° C., ethyl difluoroacetate (48.3 ml, 436 mmol) was added at such a rate that the reaction temperature did not exceed −45° C. After stirring for 5 min the mixture was poured onto 10% aq. NH₄Cl and TBME. The organic phase was washed with 5% aq. NaHCO3, brine and dried with MgSO₄.H₂O. The solvents were distilled off at atmospheric pressure and the residual product was distilled at 12 mmHg. The fraction boiling at 89-90° C. was collected to give 78.76 g of a colorless liquid.

¹H-NMR (CDCl₃, 400 MHz): δ 7.73 (t, 1H), 7.49 (q, 1H), 7.27 (m, 1H), 7.40 (t, J=54 Hz, 1H).

### b) (S)-2-(2,3-Difluoro-phenyl)-1,1-difluoro-3-nitro-propan-2-ol

A solution of 1-(2,3-difluoro-phenyl)-2,2-difluoro-ethanone (21.8 g, 113 mmol) and nitromethane (61.2 ml, 1.135 mol) in 220 ml DCM was cooled to −25° C. Catalyst 1 (3.12 g, 5.67 mmol) was added while stirring. The homogeneous solution was stored at −20° C. for 4 days. The catalyst was removed by chromatography on a small column of silica gel (DCM/(10% aq. NH₃/EtOH) 99:1). Evaporation of the solvents gave 30.45 g crude product as a colorless oil. The product was further purified by chromatography on silica gel (hexanes/DCM 50-100%) to give 27.9 g of the title compound as a colorless oil. α_{[D]}=+13.4° (c=1, CHCl₃); HPLC: Rt_{H3}=2.055 min;

¹H-NMR (CDCl₃, 400 MHz): δ 7.52 (t, 1H), 7.33-7.20 (m, 2H), 6.00 (t, J=54 Hz, 1H), 5.30 (d, 1H), 5.01 (d, 1H), 4.21 (s, 1H).

### c) (S)-3-Amino-2-(2,3-difluoro-phenyl)-1,1-difluoro-propan-2-ol

A solution of (S)-2-(2,3-difluoro-phenyl)-1,1-difluoro-3-nitro-propan-2-ol (27.97 g, 110 mmol) in 90 ml AcOH was added dropwise to a well-stirred suspension of Zn (72.3 g, 1.105 mol) powder in 200 ml AcOH. The reaction temperature was kept at 35-45° C. After the addition the mixture was stirred rt 1 h, filtered over celite and washed with EtOAc. The filtrate and EtOAc washings were evaporated, the residue dissolved in EtOAc and so much 1N aq. NaOH was added till the pH of the aq. layer had reached ca. 12. Insoluble parts were dissolved through the addition of a little sat. aq. NH₃. The organic layer was washed with brine, dried with MgSO₄.H₂O and evaporated. The residue was crystallized from TBME/hexanes to provide 22.4 g of the title compound as white crystals. HPLC: Rt_{H1}=2.469 min [M+H]⁺224;

¹H-NMR (DMSO-d6, 400 MHz): δ 7.46-7.37 (m, 2H), 7.21 (q, 1H), 6.16 (t, J=54 Hz, 1H), 6.1 (br, 1H), 3.06 (d, 1H), 3.02 (d, 1H), 4.21 (s, 1H).

### d) N—[(S)-2-(2,3-Difluoro-phenyl)-3,3-difluoro-2-hydroxy-propyl]-2-nitro-benzenesulfonamide

A solution of (S)-3-amino-2-(2,3-difluoro-phenyl)-1,1-difluoro-propan-2-ol (22.4 g, 100 mmol) and pyridine (40.6 ml, 502 mmol) in 230 ml DCM was cooled at +5° C. 2-nitro-benzenesulfonyl chloride (23.36 g, 105 mmol) was added in portions (T<15° C.). After the addition the mixture was stirred without ice-cooling for 1 h. The mixture was diluted with TBME and 2N HCl. The organic layer was washed with brine and dried with MgSO₄.H₂O and evaporated. The crude product was purified by chromatography on silica gel (hexanes/DCM 15-30%, then DCM/EtOH 0-3%) to give 39.6 g of the title compound as a yellow resin that crystallized upon standing.

HPLC: Rt_{H3}=2.644 min [M+Na]⁺409;

¹H-NMR (CDCl₃, 400 MHz): δ 8.10 (m, 2H), 7.86 (m, 1H), 7.76 (m, 2H), 7.38 (t, 1H), 7.19-7.07 (m, 2H), 6.03 (t, J=54 Hz, 1H), 5.67 (t, 1H), 3.88 (dd, 1H), 3.73 (dd, 1H), 3.41 (s, 1H).

### e) (R)-2-Difluoromethyl-2-(2,3-difluoro-phenyl)-1-(2-nitro-benzenesulfonyl)-aziridine

N—[(S)-2-(2,3-Difluoro-phenyl)-3,3-difluoro-2-hydroxy-propyl]-2-nitro-benzenesulfonamide (39.65 g, 97 mmol) was dissolved in 400 ml THF together with PPh₃ (30.6 g, 117 mmol), cooled to 0-5° C. and treated with a 40% toluene solution of DEAD (53.4 ml, 117 mmol) in a dropwise manner. Stirring was continued for 3 h while slowly warming to rt. The solution was diluted with 400 ml toluene, concentrated to remove the THF and directly purified via chromatography on silica gel (hexanes/DCM 50-70%) to give the title compound as a yellow resin.

HPLC: Rt_{H3}=3.096 min [M+Na]⁺413;

¹H-NMR (CDCl₃, 400 MHz): δ 8.28-8.23 (m, 1H), 7.83-7.75 (m, 3H), 7.40 (t, 1H), 7.30-7.21 (m, 1H), 7.19-7.12 (m, 1H), 6.17 (t, J=54 Hz, 1H), 3.38 (s, 1H), 3.27 (s, 1H).

### f) Acetic acid (R)-2-(2,3-difluoro-phenyl)-3,3-difluoro-2-(2-nitro-benzenesulfonylamino)-propyl ester

A solution of (R)-2-difluoromethyl-2-(2,3-difluoro-phenyl)-1-(2-nitro-benzenesulfonyl)-aziridine (4.78 g, 12.25 mmol) in 50 ml DMSO was treated with KOAc (2.404 g, 24.49 mmol) and stirred for 2 h. The mixture was diluted with EtOAc, washed with water twice followed by brine and dried with MgSO₄.H₂O. The crude product was purified by chromatography on silica gel (hexanes/EtOAc 25-35%) to give 4.6 g of the title compound as a colorless resin.

HPLC: Rt_{H3}=2.906 min [M+Na]⁺473;

¹H-NMR (CDCl₃, 400 MHz): δ 7.99 (d, 1H), 7.77-7.71 (m, 1H), 7.57 (m, 2H), 7.37-7.31 (m, 1H), 7.23-7.15 (m, 2H), 6.70 (s, 1H), 6.59 (t, J=54 Hz, 1H), 4.57 (d, 1H), 4.55 (d, 1H), 2.10 (s, 3H).

### g) N—[(R)-1-(2,3-Difluoro-phenyl)-2,2-difluoro-1-hydroxymethyl-ethyl]-2-nitro-benzenesulfonamide

A solution of acetic acid (R)-2-(2,3-difluoro-phenyl)-3,3-difluoro-2-(2-nitro-benzenesulfonylamino)-propyl ester (4.57 g, 10.15 mmol) in 35 ml MeOH was treated with aq LiOH (4M, 12.68 ml, 50.7 mmol). The reaction was slightly exothermic. After 30 min the mixture was diluted with water brine and EtOAc. The organic layer was washed with 1N HCl and brine, and dried with MgSO₄.H₂O. Evaporation gave the title compound as a white solid, pure enough for further transformations.

HPLC: Rt_{H3}=2.516 min [M+Na]⁺431;

¹H-NMR (DMSO-d6, 400 MHz): δ 8.67 (s, 1H), 7.91 (d, 1H), 7.80 (t, 1H), 7.74-7.67 (m, 2H), 7.37 (q, 1H), 7.30-7.24 (m, 1H), 7.19-7.12 (m, 1H), 6.69 (t, J=54 Hz, 1H), 5.44 (t, 1H), 3.98 (s, 2H), 2.10 (s, 3H).

### h) [(R)-2-(2,3-Difluoro-phenyl)-3,3-difluoro-2-(2-nitro-benzenesulfonylamino)-propoxy]-acetic acid ethyl ester

To a solution of N—[(R)-1-(2,3-difluoro-phenyl)-2,2-difluoro-1-hydroxymethyl-ethyl]-2-nitro-benzenesulfonamide (2.59 g, 6.34 mmol) and rhodium(II)acetate, dimer (0.056 g, 0.127 mmol) in 41 ml DCM was added ethyl diazoacetate (1.570 ml, 12.69 mmol) in 7.4 ml DCM over a period of 4 h using a syringe pump. The mixture was stirred for 1 h, diluted with hexanes and chromatographed on a silica gel column (hexanes/DCM 50-100%) to give 1.78 g of the title compound as a slightly impure pale yellow resin.

HPLC: Rt_{H4}=2.784 min [M+Na]⁺517;

¹H-NMR (CDCl₃, 400 MHz): δ 7.95 (d, 1H), 7.70 (t, 1H), 7.60 (d, 1H), 7.54 (t, 1H), 7.46 (t, 1H), 7.20-7.05 (m, 2H), 6.97 (s, 1H), 6.61 (t, J=54 Hz, 1H), 4.34-4.08 (m, 6H), 1.37-1.27 (m, 3H).

### i) (R)-5-Difluoromethyl-5-(2,3-difluoro-phenyl)-morpholin-3-one

A solution of [(R)-2-(2,3-difluoro-phenyl)-3,3-difluoro-2-(2-nitro-benzenesulfonylamino)-propoxy]-acetic acid ethyl ester (2.46 g, 4.98 mmol) in 25 ml MeOH was treated with aq. LiOH (4M, 6.22 ml, 24.88 mmol). The reaction was slightly exothermic. After 30 min the mixture was diluted with 1N HCl, brine and EtOAc. The organic layer was washed with brine and dried with MgSO₄.H₂O. Evaporation gave the title compound as a yellow resin, used for further transformations without purification. HPLC: Rt_{H3}=2.575 min [M+Na]⁺489.

The product was dissolved in 12 ml EtOH and 6 ml THF, treated with thiophenol (1.1 g, 10 mmol) and 1 M NaOH (14.9 ml), and heated at 60° C. for 4 h. The mixture was cooled down and washed with TBME. The pH was adjusted to 6-7 with 1N HCl and evaporated to dryness. The residual product was extracted with EtOH (3×). The ethanol extracts were evaporated to give 1.69 g of a yellow foam. HPLC: Rt_{H1}=3.478 min [M+H]⁺282. This product was refluxed in 50 ml toluene containing 2.5 ml AcOH for 18 h. The mixture was evaporated and the title compound was isolated as a white solid after chromatography on silica gel (hexanes/EtOAc 25-40%).

HPLC: Rt_{H2}=2.673 min [M+H]⁺264;

¹H-NMR (CDCl₃, 400 MHz): δ 7.33-7.19 (m, 4H), 6.68 (br s, 1H), 6.34 (t, J=54 Hz, 1H), 4.34-4.18 (m, 4H).

### j) (R)-5-Difluoromethyl-5-(2,3-difluoro-phenyl)-morpholine-3-thione

To a solution of (R)-5-difluoromethyl-5-(2,3-difluoro-phenyl)-morpholin-3-one (543 mg, 2.063 mmol) in 6 ml THF was added Lawesson's reagent (459 mg, 1.135 mmol) and the mixture was stirred at 50° C. for 45 min. The mixture was evaporated and purified by chromatography on silica gel (hexanes/EtOAc 10-15%) to give 587 mg of the title compound as a colorless resin.

HPLC: Rt_{H2}=3.124 min [M+H]⁺280;

¹H-NMR (CDCl₃, 400 MHz): δ 8.43 (br s, 1H), 7.33-7.19 (m, 3H), 7.12 (t, 1H), 6.34 (t, J=54 Hz, 1H), 4.62 (d, 1H), 4.55 (d, 1H), 4.27 (s, 2H).

### k) (R)-5-Difluoromethyl-5-(2,3-difluoro-phenyl)-5,6-dihydro-2H-[1,4]oxazin-3-ylamine

A solution of (R)-5-difluoromethyl-5-(2,3-difluoro-phenyl)-morpholine-3-thione in a NH₃/MeOH solution (7 mol/L, 8.5 ml) was stirred in a sealed vessel for 4 h. The mixture was evaporated and chromatographed on silica gel (DCM/(EtOH/sat aq NH₃ 9:1) 0-5%) to yield 517 mg of the title compound as a colorless resin. HPLC: Rt_{H2}=2.249 min [M+H]⁺263;

¹H-NMR (CDCl₃, 400 MHz): δ 7.51 (t, 1H), 7.24-7.12 (m, 2H), 6.34 (t, J=54 Hz, 1H), 4.38 (d, 1H), 4.35 (d, 1H), 4.19 (d, 1H), 4.03 (d, 1H).

### l) (R)-5-Difluoromethyl-5-(2,3-difluoro-5-nitro-phenyl)-5,6-dihydro-2H-[1,4]oxazin-3-ylamine

To a stirred solution of (R)-5-difluoromethyl-5-(2,3-difluoro-phenyl)-5,6-dihydro-2H-[1,4]oxazin-3-ylamine (508 mg, 1.937 mmol) in 5 ml H₂SO₄ was added KNO₃ (255 mg, 2.52 mmol) in four portions (exothermic). The resulting solution was stirred 20 minutes at rt and then poured on ice-water. The mixture was basified by addition of solid Na₂CO₃ (careful !: foaming) and extracted with EtOAc. The organic layer was washed with brine, treated with some charcoal and MgSO₄.H₂O and filtered over celite. Evaporation of the solvent gave the title compound, containing 6% of a regioisomer. The product was used without further purification.

HPLC: Rt_{H2}=2.313 min [M+H]⁺308;

¹H-NMR (CDCl₃, 400 MHz): δ 8.65 (s, 1H), 8.10 (t, 1H), 6.10 (t, J=54 Hz, 1H), 4.52-3.98 (m, 4H).

### m) [(R)-5-Difluoromethyl-5-(2,3-difluoro-5-nitro-phenyl)-5,6-dihydro-2H-[1,4]oxazin-3-yl]-carbamic acid tert-butyl ester

To a solution of (R)-5-difluoromethyl-5-(2,3-difluoro-5-nitro-phenyl)-5,6-dihydro-2H-[1,4]oxazin-3-ylamine (510 mg, 1.660 mmol) in 5 ml DCM were added DIPEA (322 mg, 2.49 mmol) and di-tert-butyldicarbonate (417 mg, 2.158 mmol). The reaction mixture was stirred overnight at 40° C. The reaction mixture was evaporated and the title compound was isolated as white crystals (TBME/hexanes). TLC (hexanes/EtOAc 6:1): Rf=0.25;

HPLC: Rt_{H3}=3.475 min; ESIMS=[M+Na]⁺430;

¹H-NMR (CDCl₃, 400 MHz): δ 8.55-8.51 (m, 1H), 8.14-8.08 (m, 1H), 7.43 (br s, 1H), 6.04 (t, J=54 Hz, 1H), 4.87 (d, 1H), 4.59 (d, 1H), 4.51 (dd, 1H), 3.95 (d, 1H), 1.52 (s, 9H).

### n) [(R)-5-(5-Amino-2,3-difluoro-phenyl)-5-difluoromethyl-5,6-dihydro-2H-[1,4]oxazin-3-yl]-carbamic acid tert-butyl ester

A solution of [(R)-5-difluoromethyl-5-(2,3-difluoro-5-nitro-phenyl)-5,6-dihydro-2H-[1,4]oxazin-3-yl]-carbamic acid tert-butyl ester (540 mg, 1.326 mmol) in 3 ml EtOH and 2 ml THF was stirred in a hydrogen atmosphere in the presence of 140 mg 5% Pd—C “degussa” E101 ND till LC-MS analysis indicated complete conversion. The mixture was flushed with nitrogen, diluted with DCM and filtered over a pad of celite. The filtrate was evaporated and further purified by chromatography on silica gel (hexanes/EtOAc 25-50%) to give the title compound as a colorless foam. TLC (hexanes/EtOAc 2:1): Rf=0.26;

HPLC: Rt_{H2}=3.057 min; ESIMS=[M+H]⁺378;

¹H-NMR (CDCl₃, 400 MHz, broad signals due to rotamers): δ 6.73 (m, 1H), 6.50 (m, 1H), 6.18 (t, J=54 Hz, 1H), 4.95-3.99 (m, 4H), 1.52 (s, 9H).

### o) ((R)-5-{5-[(5-Cyano-3-methyl-pyridine-2-carbonyl)-amino]-2,3-difluoro-phenyl}-5-difluoromethyl-5,6-dihydro-2H-[1,4]oxazin-3-yl)-carbamic acid tert-butyl ester

To an ice-cold solution of [(R)-5-(5-amino-2,3-difluoro-phenyl)-5-difluoromethyl-5,6-dihydro-2H-[1,4]oxazin-3-yl]-carbamic acid tert-butyl ester (113 mg, 0.299 mmol), 5-cyano-3-methyl-pyridine-2-carboxylic acid (53.4 mg, 0.329 mmol), HOAt (65.2 mg, 0.479 mmol) in 1.2 ml DMF were added 0.07 ml (0.39 mmol) EDC (free base). The mixture was stirred overnight at rt. Water and EtOAc were added and the organic layer was washed with sat aq NaHCO₃, brine and dried with MgSO₄.H₂O. The product was purified by chromatography on silica gel (hexanes/EtOAc 15-20%) to give 108 mg of the title compound as colorless foam. TLC (hexanes/EtOAc 3:1): Rf=0.31; HPLC: Rt_{H3}=3.374 min; ESIMS=[M+H]⁺522;

¹H-NMR (CDCl₃, 400 MHz): δ 10.12 (s, 1H), 8.76 (s, 1H), 8.20 (br t, 1H), 7.99 (s, 1H), 7.40 (br s, 1H), 6.13 (t, J=54 Hz, 1H), 4.85 (d, 1H), 4.62 (d, 1H), 4.43 (d, 1H), 4.40 (d, 1H), 2.89 (s, 3H), 1.52 (s, 9H).

### p) 5-Cyano-3-methyl-pyridine-2-carboxylic acid [3-((R)-5-amino-3-difluoromethyl-3,6-dihydro-2H-[1,4]oxazin-3-yl)-4,5-difluoro-phenyl]-amide

To a solution of ((R)-5-{5-[(5-cyano-3-methyl-pyridine-2-carbonyl)-amino]-2,3-difluoro-phenyl}-5-difluoromethyl-5,6-dihydro-2H-[1,4]oxazin-3-yl)-carbamic acid tert-butyl ester (107 mg, 0.205 mmol) in 0.9 ml DCM were added dropwise 0.3 ml TFA. The mixture was stirred 1.5 h at rt. The reaction mixture was carefully poured onto ca 10% aq. soda and EtOAc. The organic phase was washed with sat aq NaHCO₃ and brine, and dried with Na₂SO₄. The product was purified by chromatography on silica gel (DCM/(EtOH/sat. aq. NH₃ 9:1) 0-2%) to give 81 mg of the title compound as white solid.

HPLC: Rt_{H2}=2.827 min; ESIMS [M+H]⁺=422;

¹H-NMR (DMSO-d6, 600 MHz): 10.91 (s, 1H), 8.40 (s, 1H) 8.98-8.94 (m, 1H), 7.82 (s, 1H), 6.19 (s, 2H), 6.13 (t, J=54 Hz, 1H), 4.08 (d, 1H), 4.01 (d, 1H), 3.95 (d, 1H), 3.89 (d, 1H), 2.53 (s, 3H).

### Examples 229 to 230

The compounds listed in Table 23 were prepared by a procedure analogous to those used in Example 228.

Hydrochloride salts were obtained from solutions of the corresponding free base by addition of hydrochloric acid in dioxane or hydrochloric acid in diethylether and evaporation of the solvents.

TABLE 23

| | | | MS |
|---|---|---|---|
| | | ¹H-NMR | [m/z; |
| Example | Compound | (δ; DMSO-d₆) | (M + 1)⁺] |
| 229 | 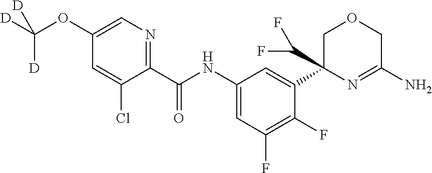 [SMILES: [2H]C([2H])([2H])Oc1cnc(C(=O)Nc2cc(F)c(F)c([C@]3(C(F)F)COCC(N)=N3)c2)c(Cl)c1] | 10.91 (s, 1H), 8.40 (s, 1H) 8.98-8.94 (m, 1H), 7.82 (s, 1H), 6.19 (s, 2H), 6.13 (t, J = 54 Hz, 1H), 4.08 (d, 1H), 4.01 (d, 1H), 3.95 (d, 1H), 3.89 (d, 1H), 2.53 (s, 3H). | 422 |
| | 3-Chloro-5-trideuteromethoxy-pyridine-2- | | |
| | carboxylic acid [3-((R)-5-amino-3- | | |
| | difluoromethyl-3,6-dihydro-2H-[1,4]oxazin-3- | | |
| | yl)-4,5-difluoro-phenyl]-amide | | |
| 230 | 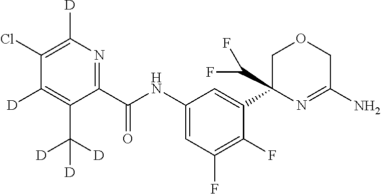 [SMILES: [2H]c1nc(C(=O)Nc2cc(F)c(F)c([C@]3(C(F)F)COCC(N)=N3)c2)c(C([2H])([2H])[2H])c([2H])c1Cl] | 10.78 (s, 1H), 7.98- 7.93 (m, 1H) 7.83 (s, 1H), 6.19 (s, 2H), 6.14 (t, J = 54 Hz, 1H), 4.10 (d, 1H), 4.02 (d, 1H), 3.91 (d, 1H), 3.88 (d, 1H). | 436 |
| | 4,6-Dideutero-5-chloro-3-trideuteromethyl- | | |
| | pyridine-2-carboxylic acid [3-((R)-5-amino-3- | | |
| | difluoromethyl-3,6-dihydro-2H-[1,4]oxazin-3- | | |
| | yl)-4,5-difluoro-phenyl]-amide | | |

### Example 231

### 3-Chloro-5-cyano-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide hydrochloride

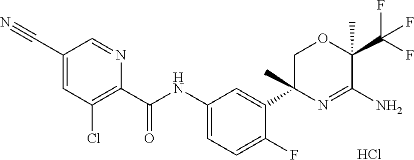 [SMILES: C[C@@]1(c2cc(NC(=O)c3ncc(C#N)cc3Cl)ccc2F)CO[C@@](C)(C(F)(F)F)C(N)=N1 ; Cl]

### a) 2-(5-Bromo-2-fluoro-phenyl)-propan-2-ol

To a solution of diisopropyl amine (57.3 ml, 402 mmol) in THF (500 ml) was added under argon a 1.6 M solution of nBuLi in hexane (260 ml, 416 mmol) below −50° C. After stirring for 30 min at −75° C., 4-bromo-1-fluoro benzene (31.1 ml, 277 mmol) was added while keeping the temperature below −70° C. After stirring for 2 h at −75° C., acetone (41.2 ml, 554 mmol) was added below −65° C. and the reaction mixture was stirred for 1 h at −75° C., warmed up to −50° C. and poured onto 10% aqueous NH₄Cl solution. The mixture was extracted with TBME, organic phases were washed with aqueous KHSO₄ solution, saturated NaHCO₃ solution and brine, dried over MgSO₄, filtered and concentrated. The crude product was crystallized from hexane to provide the title compound as white crystals: TLC (hexane-EtOAc 3:1): Rf=0.45; HPLC: Rt_{H5}=1.045 min; ¹H-NMR (360 MHz, CDCl₃): δ 7.74 (dd, 1H), 7.36 (m, 1H), 6.93 (dd, 1H), 2.04 (d, 1H), 1.63 (s, 6H).

### b) 4-Bromo-1-fluoro-2-isopropenyl-benzene

To a solution of 2-(5-bromo-2-fluoro-phenyl)-propan-2-ol (119.7 g, 498 mmol) in CH₂Cl₂ (50 ml) was added hydrochinone (2.74 g, 24.9 mmol) and 250 ml 85% H₃PO₄. The resulting reaction mixture was stirred for 3.5 h at 50° C. The mixture was poured onto ice-water and extracted with CH₂Cl₂. The organic phases were washed with 2N aqueous NaOH and water, dried over MgSO₄, filtered and concentrated. The crude product was dissolved in hexane and filtered through a plough of silica gel to obtain after concentration at 600 mbar the title compound as a colorless oil: TLC (hexane): Rf=0.52; HPLC: Rt_{H5}=1.416 min; ¹H-NMR (360 MHz, CDCl₃): δ 7.43 (dd, 1H), 7.37 (m, 1H), 6.94 (dd, 1H), 5.27 (d, 2H), 2.13 (s, 3H).

### c) (S)-2-(5-Bromo-2-fluoro-phenyl)-propane-1,2-diol

To a suspension of K₃Fe(CN)₆ (186 g, 561 mmol), K₂CO₃ (78 g, 561 mmol), (DHQ)₂-PHAL (1.311 g, 1.674 mmol) and K₂OsO₂(OH)₄ (0.378 g, 1 mmol) in t-BuOH—H₂O 1:1 (1600 ml) was added 4-bromo-1-fluoro-2-isopropenyl-benzene (36 g, 167 mmol) at 0° C. and the reaction mixture was stirred for 14 h at 0° C. After careful addition of Na₂S₂O₅ (100 g) at 0-5° C. the reaction mixture was stirred for 1 h before extraction with EtOAc. Combined extracts were washed with 5% NaS₃O₃ solution and brine, dried over MgSO4, filtered and concentrated to give the title compound as a white solid: TLC (hexane-EtOAc 1:1): Rf=0.46; HPLC: Rt_{H5}=0.767 min; ¹H-NMR (360 MHz, CDCl₃): δ 7.71 (dd, 1H), 7.27 (m, 1H), 6.83 (dd, 1H), 3.85 (d, 1H), 3.62 (d, 1H), 2.94 (s, 3H), 2.01 (s, 1H), 1.43 (s, 3H); ESIMS: 266, 268 [(M+NH₄)⁺].

### d) (S)-2-(5-Bromo-2-fluoro-phenyl)-2-methyl-oxirane

To a solution of (S)-2-(5-bromo-2-fluoro-phenyl)-propane-1,2-diol (37.35 g, 150 mmol) in CH₂Cl₂ (400 ml) was added under argon NEt₃ (41.8 ml, 300 mmol) and dropwise mesyl chloride (12.8 ml, 165 mmol) at 0-5° C. After stirring for 0.5 h at 0-5° C. the reaction mixture was added to cold 1N HCl and extracted with CH₂Cl₂. Combined extracts were washed with 1N HCl, H₂O and saturated NaHCO₃ solution, dried over MgSO₄, filtered and concentrated. The crude mesylate was dissolved in TBME (500 ml) and 200 ml 2N aqueous NaOH and after stirring for 2 h at 25° C. the mixture was extracted with TBME. Combined extracts were washed with NaH₂PO₄ solution and brine, dried over MgSO₄, filtered and concentrated to provide the (S)-enantiomer as a colorless oil: 78% ee (Chiralpak AS-H 1218, hexane-EtOH 97:3, 0.4 mL/min); TLC (hexane-EtOAc 3:1): Rf=0.69; HPLC: Rt_{H5}=1.186 min; ¹H-NMR (360 MHz, CDCl₃): δ 7.46 (dd, 1H), 7.30 (m, 1H), 6.83 (dd, 1H), 2.88 (d, 1H), 2.72 (d, 1H), 1.59 (s, 3H).

### e) (S)-1-Azido-2-(5-bromo-2-fluoro-phenyl)-propan-2-ol

To a solution of (S)-2-(5-bromo-2-fluoro-phenyl)-2-methyl-oxirane (51.85 g, 224 mmol) in EtOH (800 ml) was added NaN₃ (36.8 g, 531 mmol), NH₄Cl (60.6 g, 1122 mmol) and 18-crown-6 (59.8 g, 224 mmol) and the reaction mixture was heated at reflux for 6 h. The reaction mixture was filtered and concentrated to half of its volume. The residual oil was extracted with EtOAc. Combined extracts were washed with saturated NaHCO₃ solution and brine, dried over MgSO₄, filtered and concentrated to provide the title compound as a light yellow oil: TLC (hexane-EtOAc 1:1): Rf=0.70; HPLC: Rt_{H3}=1.115 min; ¹H-NMR (360 MHz, CDCl₃): δ 7.72 (dd, 1H), 7.32 (m, 1H), 6.85 (dd, 1H), 3.73 (d, 1H), 3.51 (d, 1H), 2.44 (s, 1H), 1.50 (s, 3H).

### f) (S)-1-Amino-2-(5-bromo-2-fluoro-phenyl)-propan-2-ol

To a suspension of LiAlH₄ (4.65 g, 122 mmol) in THF (250 ml) was added under argon at 0-5° C. a solution of (S)-1-azido-2-(5-bromo-2-fluoro-phenyl)-propan-2-ol (33.4 g, 122 mmol) dissolved in THF (150 ml) over a period of 30 min. After stirring for 1 h at 0-5° C., the reaction was quenched by careful addition of water (4.7 ml), 4 N NaOH (4.7 ml) and water (14.1 ml) and stirred again for 3 h at 25° C. The white suspension was dried with MgSO₄, filtered and concentrated. The solidified product was re-crystallized from TBME-hexane to provide the title compound as beige crystals: 98% ee (Chiralpak AD-H hexane-EtOH 75-25+0.05% NEt₃); TLC (CH₂Cl₂-MeOH 10:1) Rf=0.10; HPLC: Rt_{H5}=0.558 min; ESIMS: 248, 250 [(M+H)⁺]; ¹H-NMR (360 MHz, CDCl₃): δ 7.76 (dd, 1H), 7.25 (m, 1H), 6.82 (dd, 1H), 4.16 (br s, 1H), 3.19 (d, 1H), 2.72 (d, 1H), 1.44 (s, 3H), 0.95 (br s, 2H).

### g) N—[(S)-2-(5-Bromo-2-fluoro-phenyl)-2-hydroxy-propyl]-2-nitro-benzenesulfonamide

To a solution of (S)-1-amino-2-(5-bromo-2-fluoro-phenyl)-propan-2-ol (34.7 g, 140 mmol) in THF (400 ml) was added 2-nitro-benzenesulfonyl chloride (34.9 g, 154 mmol) at 0-5° C. and afterwards 1N aqueous NaOH over a period of 0.5 h. The reaction mixture was stirred for 2 h at 20° C. The reaction mixture was diluted with TBME and washed with water and NaH₂PO₄ solution and brine, dried over MgSO₄, filtered and concentrated to provide the title compound after crystallization from TBME-hexane as beige crystals: TLC (toluene-EtOAc 3:1): Rf=0.51; HPLC: Rt_{H5}=1.118 min; ESIMS: 450, 452 [(M+NH₄)⁺]; ¹H-NMR (360 MHz, CDCl₃): δ 7.98 (m, 1H), 7.81 (m, 1H), 7.65 (m, 2H), 7.59 (dd, 1H), 7.24 (m, 1H), 6.79 (dd, 1H), 5.60 (t, 1H), 4.16 (br s, 1H), 3.55 (dd, 1H), 3.44 (dd, 1H), 2.51 (s, 1H), 1.51 (s, 3H).

### h) (R)-2-(5-Bromo-2-fluoro-phenyl)-2-methyl-1-(2-nitro-benzenesulfonyl)-aziridine

To a solution of N—[(S)-2-(5-bromo-2-fluoro-phenyl)-2-hydroxy-propyl]-2-nitro-benzene-sulfonamide (20.8 g, 48 mmol) in CH₂Cl₂ (400 ml) was added PPh₃ (19.2 g, 72.4 mmol) at 0-5° C. and diethyl azodicarboxylate (11.6 ml, 72.4 mmol). The reaction mixture was stirred for 24 h at 25° C. and concentrated. The title compound was obtained after chromatographic purification over silica gel (hexane-EtOAc 20:1 to 2:1) as yellow crystals: TLC (toluene-EtOAc 3:1): Rf=0.69; HPLC: Rt_{H5}=1.308 min; ¹H-NMR (360 MHz, CDCl₃): δ 8.31 (m, 1H), 7.28 (m, 3H), 7.60 (dd, 1H), 7.42 (m, 1H), 6.91 (dd, 1H), 3.24 (s, 1H), 2.81 (s, 1H), 2.06 (s, 3H).

### i) (R)-2-[(R)-2-(5-Bromo-2-fluoro-phenyl)-2-(2-nitro-benzenesulfonylamino)-propoxy]-3,3,3-trifluoro-2-methyl-propionic acid ethyl ester

To a suspension of NaH (2.53 g 60% in mineral oil, 63 mmol) in DMF (160 ml) was added drop-wise under argon (R)-3,3,3-trifluoro-2-hydroxy-2-methyl-propionic acid ethyl ester (11.99 g, 63 mmol) and after stirring for 0.5 h at 20° C. (R)-2-(5-bromo-2-fluoro-phenyl)-2-methyl-1-(2-nitro-benzenesulfonyl)-aziridine (21.85 g, 52.6 mmol). The reaction was kept at 25° C. for 16 h. The mixture was added to cold aqueous 2N HCl and the product extracted with TBME. Combined organic layers were washed with saturated NaHCO₃ solution and brine, dried over MgSO₄, filtered and concentrated. The residual solid was re-crystallized from TBME-hexane to provide the title compound as yellow crystals: TLC (hexane-EtOAc 1:1): Rf=0.59; HPLC: Rt_{H5}=1.444 min; ESIMS: 618, 620 [(M+NH₄)⁺]; ¹H-NMR (360 MHz, CDCl₃): δ 7.83 (dd, 1H), 7.61 (m, 3H), 7.48 (dd, 1H), 7.27 (m, 1H), 6.73 (s, 1H), 6.60 (dd, 1H), 4.33 (m, 2H), 3.84 (s, 2H), 1.84 (s, 3H), 1.57 (s, 3H), 1.33 (t, 3H).

### j) (R)-2-[(R)-2-(5-Bromo-2-fluoro-pheyl)-2-(2-nitro-benzenesulfonylamino)-propoxy]-3,3,3-trifluoro-2-methyl-propionamide

A solution of (R)-2-[(R)-2-(5-bromo-2-fluoro-phenyl)-2-(2-nitro-benzenesulfonylamino)-propoxy]-3,3,3-trifluoro-2-methyl-propionic acid ethyl ester (26.6 g, 44.2 mmol) in 7N NH₃ in MeOH (75 ml) was stirred for 16 h at 50° C. The solvent was removed under reduced pressure and the residual solid re-crystallized from Et₂O to give the title compound as yellow crystals: TLC (hexane-EtOAc 1:1): Rf=0.35; HPLC: Rt_{H5}=1.184 min; ESIMS: 589, 591 [(M+NH₄)⁺]; ¹H-NMR (360 MHz, CDCl₃): δ 7.85 (d, 1H), 7.64 (m, 3H), 7.44 (d, 1H), 7.41 (dd, 1H), 7.26 (m, 1H), 6.68 (br s, 1H), 6.57 (dd, 1H), 6.19 (s, 1H), 5.54 (br s, 1H), 4.24 (d, 1H), 3.93 (d, 1H), 1.79 (s, 3H), 1.67 (s, 3H).

### k) N—[(R)-1-(5-Bromo-2-fluoro-phenyl)-2-((R)-1-cyano-2,2,2-trifluoro-1-methyl-ethoxy)-1-methyl-ethyl]-2-nitro-benzenesulfonamide

To a solution of (R)-2-[(R)-2-(5-bromo-2-fluoro-pheyl)-2-(2-nitro-benzenesulfonylamino)-propoxy]-3,3,3-trifluoro-2-methyl-propionamide (20.83 g, 35.6 mmol) in CH₂Cl₂ (300 ml) was added under argon NEt₃ (12.5 ml, 89 mmol) and at 0-5° C. trifluoroacetic anhydride (6.15 ml, 42.7 mmol). After stirring for 4 h at 25° C. the reaction mixture was added to a cold NaHCO₃ solution and the product was extracted with CH₂Cl₂. Combined extracts were washed with cold 0.1 N aqueous HCl, water and saturated NaHCO₃ solution, dried over MgSO₄, filtered and concentrated to provide the title compound as a yellow oil, which was used as such for the next step: TLC (hexane-EtOAc 1:1): Rf=0.73; HPLC: Rt_{H5}=1.364 min; ESIMS: 571, 573 [(M+NH₄)⁺]; ¹H-NMR (360 MHz, CDCl₃): δ 7.89 (d, 1H), 7.62 (ddd, 1H), 7.57 (ddd, 1H), 7.52 (m, 2H), 7.29 (m, 1H), 6.58 (dd, 1H), 6.19 (s, 1H), 4.17 (s, 2H), 1.81 (s, 3H), 1.72 (s, 3H).

### l) (2R,5R)-5-(5-Bromo-2-fluoro-phenyl)-2,5-dimethyl-2-trifluoromethyl-5,6-dihydro-2H-[1,4]oxazin-3-ylamine

To a solution of N—[(R)-1-(5-bromo-2-fluoro-phenyl)-2-((R)-1-cyano-2,2,2-trifluoro-1-methyl-ethoxy)-1-methyl-ethyl]-2-nitro-benzenesulfonamide (6.54 g, 11.8 mmol) and N-acetyl-cysteine (2.4 g, 26.0 mmol) in MeOH (80 ml) was added K₂CO₃ (3.62 g, 26.0 mmol) and the reaction mixture was heated at 80° C. for 16 h. After removal of the solvent the residue was dissolved in water and extracted with EtOAc. Combined extracts were washed with saturated NaHCO₃ solution and brine, dried over MgSO₄, filtered and concentrated to provide the title compound after chromatographic purification over silica gel (hexane-EtOAc 10:1 to 1:2 containing 0.03% NEt₃) as a yellow oil: TLC (hexane-EtOAc 1:1): Rf=0.58; HPLC: Rt_{H5}=0.843 min; ESIMS: 369, 371 [(M+H)⁺]; ¹H-NMR (360 MHz, CDCl₃): δ 7.66 (dd, 1H), 7.35 (m, 1H), 6.91 (dd, 1H), 3.97 (m, 2H), 1.53 (s, 3H), 1.49 (s, 3H).

### m) (2R,5R)-5-(2-Fluoro-phenyl)-2,5-dimethyl-2-trifluoromethyl-5,6-dihydro-2H-[1,4]oxazin-3-ylamine

A solution of (2R,5R)-5-(5-bromo-2-fluoro-phenyl)-2,5-dimethyl-2-trifluoromethyl-5,6-dihydro-2H-[1,4]oxazin-3-ylamine (1.66 g, 4.5 mmol) and sodium acetate (0.369 g, 4.5 mmol) in MeOH (50 ml) was hydrogenated over 10% Pd—C for 6 h at 50° C. The catalyst was filtered off over Celite and the filtrate was concentrated. The residue was dissolved in saturated NaHCO₃ solution and extracted with EtOAc. Combined extracts were washed with brine, dried over MgSO₄, filtered and concentrated to provide the title compound as a colorless oil: TLC (hexane-EtOAc 1:1): Rf=0.19; HPLC: Rt_{H5}=0.777 min; ESIMS: 291 [(M+H)⁺]; ¹H-NMR (360 MHz, CDCl₃): δ 7.41 (dt, 1H), 7.26 (m, 1H), 7.11 (t, 1H), 7.05 (dd, 1H), 4.11 (dd, 1H), 3.94 (dd, 1H), 1.54 (s, 3H), 1.49 (s, 3H).

### n) (2R,5R)-5-(2-Fluoro-5-nitro-phenyl)-2,5-dimethyl-2-trifluoromethyl-5,6-dihydro-2H-[1,4]oxazin-3-ylamine

To a solution of (2R,5R)-5-(2-fluoro-phenyl)-2,5-dimethyl-2-trifluoromethyl-5,6-dihydro-2H-[1,4]oxazin-3-ylamine (1.035 g, 3.57 mmol) in H₂SO₄ (6 ml) was added in portions KNO₃ (0.379 g, 3.74 mmol) under ice-water cooling. The reaction mixture was stirred for 2 h at 25° C., diluted with water and basified with K₂CO₃ under cooling. The product was extracted with EtOAc. Combined extracts were washed with saturated NaHCO₃ solution and brine, dried over MgSO₄, filtered and concentrated. Purification via chromatography on silica gel (hexane-EtOAc 4:1 to 1:1 containing 0.05% NEt₃) gave the title compound as a light yellow oil: TLC (hexane-EtOAc 1:1): Rf=0.50; HPLC: Rt_{H5}=0.749 min; ESIMS: 336 [(M+H)⁺]; ¹H-NMR (360 MHz, CDCl₃): δ 8.48 (dd, 1H), 8.14 (m, 1H), 7.15 (dd, 1H), 4.20 (br s, 2H), 4.04 (dd, 1H), 3.91 (dd, 1H), 1.54 (s, 3H), 1.49 (s, 3H).

### o) [(2R,5R)-5-(2-Fluoro-5-nitro-phenyl)-2,5-dimethyl-2-trifluoromethyl-5,6-dihydro-2H-[1,4]oxazin-3-yl]-carbamic acid tert-butyl ester

To a solution of (2R,5R)-5-(2-fluoro-5-nitro-phenyl)-2,5-dimethyl-2-trifluoromethyl-5,6-di-hydro-2H-[1,4]oxazin-3-ylamine (1.14 g, 3.4 mmol) in ACN (20 ml) was added Boc₂O (0.891 g, 4.08 mmol) and NEt₃ (0.72 ml, 5.1 mmol) and the mixture was stirred for 16 h at 25° C. The reaction mixture was evaporated and the residual oil purified by chromatography on silica gel (hexane-EtOAc 20:1 to 7:3) to give the title compound after crystallization from Et₂O-hexane as beige crystals: TLC (hexane-EtOAc 3:1): Rf=0.37; HPLC: Rt_{H5}=1.355 min; ESIMS: 436 [(M+H)⁺]; ¹H-NMR (360 MHz, CDCl₃): δ 11.04 (br s, 1H), 8.24 (m, 2H), 7.30 (dd, 1H), 4.41 (dd, 1H), 4.11 (dd, 1H), 1.68 (s, 3H), 1.51 (s, 9H), 1.49 (s, 3H).

### p) [(2R,5R)-5-(5-Amino-2-fluoro-phenyl)-2,5-dimethyl-2-trifluoromethyl-5,6-dihydro-2H-[1,4]oxazin-3-yl]-carbamic acid tert-butyl ester

A solution of [(2R,5R)-5-(2-fluoro-5-nitro-phenyl)-2,5-dimethyl-2-trifluoromethyl-5,6-dihydro-2H-[1,4]oxazin-3-yl]-carbamic acid tert-butyl ester (0.98 g, 2.25 mmol) in isopropanol-THF 2:1 (24 ml) was hydrogenated over 5% Pd—C for 4 h at 50° C. The catalyst was filtered off over Celite and the filtrate was concentrated to provide the title compound after crystallization from TBME-hexane as beige crystals: TLC (hexane-EtOAc 1:1): Rf=0.42; HPLC: Rt_{H5}=0.955 min; ESIMS: 406 [(M+H)⁺]; ¹H-NMR (360 MHz, CDCl₃): δ 6.82 (dd, 1H), 6.52 (m, 2H), 4.30 (dd, 1H), 3.97 (dd, 1H), 3.06 (br s, 2H), 1.58 (s, 3H), 1.48 (s, 3H), 1.46 (s, 9H).

### q) ((2R,5R)-5-{5-[(3-Chloro-5-cyano-pyridine-2-carbonyl)-amino]-2-fluoro-phenyl}-2,5-dimethyl-2-trifluoromethyl-5,6-dihydro-2H-[1,4]oxazin-3-yl)-carbamic acid tert-butyl ester

To a solution of [(2R,5R)-5-(5-amino-2-fluoro-phenyl)-2,5-dimethyl-2-trifluoromethyl-5,6-dihydro-2H-[1,4]oxazin-3-yl]-carbamic acid tert-butyl ester (82 mg, 0.20 mmol) in DMF (2 ml) was added 3-chloro-5-cyano-pyridine-2-carboxylic acid (47 mg, 0.26 mmol), EDC-HCl (57 mg, 0.30 mmol), HOAt (41 mg, 0.30 mmol) and DIPEA (0.14 ml, 0.79 mmol) and the reaction mixture was kept at 25° C. for 16 h. The reaction mixture was concentrated under reduced pressure, the residue dissolved in EtOAc and washed with saturated NaHCO₃ solution and brine, dried over MgSO₄, filtered and concentrated. The title compound was obtained after purification by flash column chromatography on silica gel (hexane-EtOAc 20:1 to 1:1) as a light yellow foam. TLC (hexane-EtOAc 2:1): Rf=0.29; HPLC: Rt_{H5}=1.398 min; ESIMS: 570, 572 [(M+H)⁺]; ¹H-NMR (360 MHz, CDCl₃): δ 11.05 (br s, 1H), 9.74 (br s, 1H), 8.79 (s, 1H), 8.19 (s, 1H), 7.87 (m, 1H), 7.55 (dd, 1H), 7.16 (dd, 1H), 4.43 (d, 1H), 4.09 (d, 1H), 1.71 (s, 3H), 1.57 (s, 3H), 1.56 (m, 9H); ¹⁹F-NMR (360 MHz, CDCl₃): δ 74.3, 116.2.

### r) 3-Chloro-5-cyano-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide hydrochloride

To a solution of ((2R,5R)-5-{5-[(3-Chloro-5-cyano-pyridine-2-carbonyl)-amino]-2-fluoro-phenyl}-2,5-dimethyl-2-trifluoromethyl-5,6-dihydro-2H-[1,4]oxazin-3-yl)-carbamic acid tert-butyl ester (105 mg, 0.166 mmol) in CH₂Cl₂ (1 ml) was added TFA (0.3 ml) and the reaction mixture was kept at 25° C. for 2 h. The reaction was added to cold 10% aq. K₂CO₃ solution and the product was extracted with EtOAc. Combined organic extracts were washed with brine, dried over MgSO₄, filtered and concentrated to provide 3-chloro-5-cyano-pyridine-2-carboxylic acid [3-((3R,6R)-5-amino-3,6-dimethyl-6-trifluoro-methyl-3,6-dihydro-2H-[1,4]oxazin-3-yl)-4-fluoro-phenyl]-amide as a colorless foam. The title compound was converted into its hydrochloride salt by dissolving the free base in CH₂Cl₂, adding 1.05 equivalent of 2N HCl in Et₂O, evaporation to dryness, followed by crystallization from CH₂Cl₂-Et₂O to provide the title compound as a white solid: TLC (CH₂Cl₂-MeOH 9:1): Rf=0.51; HPLC: Rt_{H5}=0.939 min; ESIMS: 470, 472 [(M+H)⁺]; ¹H-NMR (600 MHz, DMSO-d₆): δ 11.59 (s, 1H), 11.15 (s, 1H), 9.60 (d, 2H), 9.13 (s, 1H), 8.84 (s, 1H), 7.83 (m, 1H), 7.78 (dd, 1H), 7.36 (dd, 1H), 4.32 (d, 1H), 4.09 (d, 1H), 1.73 (s, 3H), 1.72 (s, 3H); ¹⁹F-NMR (360 MHz, CDCl₃): δ 76.4, 116.4.

### Examples 232 to 250

The compounds listed in Table 24 were prepared by a procedure analogous to those used in Example 231.

TABLE 24

| | | | MS |
|---|---|---|---|
| | | ¹H-NMR | [m/z; |
| Example | Compound | (δ; DMSO-d₆) | (M + 1)⁺] |
| 232 | 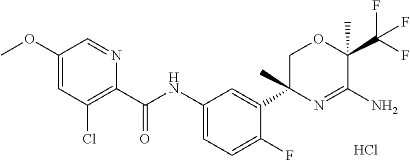 [SMILES: COc1cnc(C(=O)Nc2ccc(F)c([C@]3(C)CO[C@@](C)(C(F)(F)F)C(N)=N3)c2)c(Cl)c1 ; Cl] | 11.75 (s, 1H), 10.80 (s, 1H), 9.65 (d, 2H), 8.36 (d, 1H), 7.90 (m, 1H), 7.82 (dd, 1H), 7.75 (d, 1H), 7.32 (dd, 1H), 4.32 (d, 1H), 4.07 (d, 1H), 3.94 (s, 3H), 1.74 (s, 3H), 1.72 (s, 3H). | 475, 477 |
| | 3-Chloro-5-methoxy-pyridine-2-carboxylic | | |
| | acid [3-((3R,6R)-5-amino-3,6-dimethyl-6- | | |
| | trifluoromethyl-3,6-dihydro-2H-[1,4]oxazin-3- | | |
| | yl)-4-fluoro-phenyl]-amide hydrochloride | | |
| 233 | 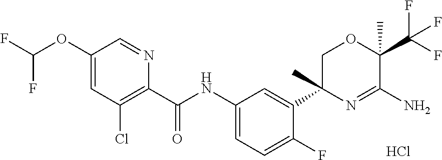 [SMILES: C[C@@]1(c2cc(NC(=O)c3ncc(OC(F)F)cc3Cl)ccc2F)CO[C@@](C)(C(F)(F)F)C(N)=N1 ; Cl] | 11.59 (s, 1H), 10.95 (s, 1H), 9.59 (d, 2H), 8.60 (d, 1H), 8.15 (d, 1H), 7.85 (m, 1H), 7.80 (d, 1H), 7.51, (t, 1H), 7.35 (dd, 1H), 4.32 (d, 1H), 4.08 (d, 1H), 1.74 (s, 3H), 1.72 (s, 3H). | 511, 513 |
| | 3-Chloro-5-difluoromethoxy-pyridine-2- | | |
| | carboxylic acid [3-((3R,6R)-5-amino-3,6- | | |
| | dimethyl-6-trifluoromethyl-3,6-dihydro-2H- | | |
| | [1,4]oxazin-3-yl)-4-fluoro-phenyl]-amide | | |
| | hydrochloride | | |
| 234 | 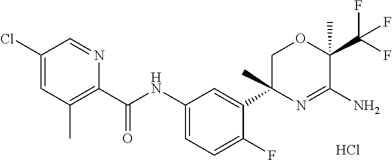 [SMILES: Cc1cc(Cl)cnc1C(=O)Nc1ccc(F)c([C@]2(C)CO[C@@](C)(C(F)(F)F)C(N)=N2)c1 ; Cl] | 11.55 (s, 1H), 10.84 (s, 1H), 9.55 (d, 2H), 8.61 (d, 1H), 8.06 (d, 1H), 7.96 (m, 1H), 7.87 (d, 1H), 7.33 (dd, 1H), 4.32 (d, 1H), 4.08 (d, 1H), 2.57 (s, 3H), 1.75 (s, 3H), 1.72 (s, 3H). | 459, 461 |
| | 5-Chloro-3-methyl-pyridine-2-carboxylic acid | | |
| | [3-((3R,6R)-5-amino-3,6-dimethyl-6- | | |
| | trifluoromethyl-3,6-dihydro-2H-[1,4]oxazin-3- | | |
| | yl)-4-fluoro-phenyl]-amide hydrochloride | | |
| 235 | 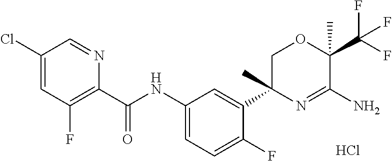 [SMILES: C[C@@]1(c2cc(NC(=O)c3ncc(Cl)cc3F)ccc2F)CO[C@@](C)(C(F)(F)F)C(N)=N1 ; Cl] | 11.62 (s, 1H), 10.98 (s, 1H), 9.59 (d, 2H), 8.69 (s, 1H), 8.36 (d, 1H), 7.93 (m, 1H), 7.88 (d, 1H), 7.35 (dd, 1H), 4.33 (d, 1H), 4.08 (d, 1H), 1.75 (s, 3H), 1.72 (s, 3H). | 463, 465 |
| | 5-Chloro-3-fluoro-pyridine-2-carboxylic acid | | |
| | [3-((3R,6R)-5-amino-3,6-dimethyl-6- | | |
| | trifluoromethyl-3,6-dihydro-2H-[1,4]oxazin-3- | | |
| | yl)-4-fluoro-phenyl]-amide hydrochloride | | |
| 236 | 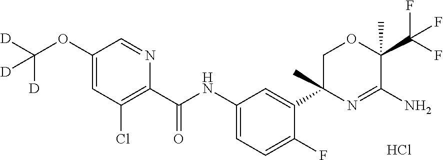 [SMILES: [2H]C([2H])([2H])Oc1cnc(C(=O)Nc2ccc(F)c([C@]3(C)CO[C@@](C)(C(F)(F)F)C(N)=N3)c2)c(Cl)c1 ; Cl] | 11.61 (s, 1H), 10.80 (s, 1H), 9.59 (d, 2H), 8.37 (d, 1H), 7.88 (m, 1H), 7.83 (m, 1H), 7.76 (d, 1H), 7.33 (dd, 1H), 4.32 (d, 1H), 4.08 (d, 1H), 1.74 (s, 3H), 1.72 (s, 3H). | 478, 480 |
| | 3-Chloro-5-trideutero-methoxy-pyridine-2- | | |
| | carboxylic acid [3-((3R,6R)-5-amino-3,6- | | |
| | dimethyl-6-trifluoromethyl-3,6-dihydro-2H- | | |
| | [1,4]oxazin-3-yl)-4-fluoro-phenyl]-amide | | |
| | hydrochloride | | |
| 237 | 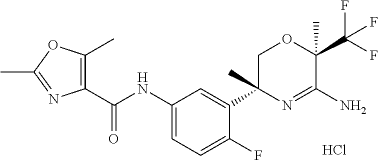 [SMILES: Cc1nc(C(=O)Nc2ccc(F)c([C@]3(C)CO[C@@](C)(C(F)(F)F)C(N)=N3)c2)c(C)o1 ; Cl] | 11.59 (s, 1H), 10.31 (s, 1H), 9.55 (d, 2H), 7.96 (m, 1H), 7.86 (dd, 1H), 7.27 (dd, 1H), 4.30 (d, 1H), 4.05 (d, 1H), 2.58 (s, 3H), 2.46 (s, 3H), 1.75 (s, 3H), 1.70 (s, 3H). | 429 |
| | 2,5-Dimethyl-oxazole-4-carboxylic acid [3- | | |
| | ((3R,6R)-5-amino-3,6-dimethyl-6- | | |
| | trifluoromethyl-3,6-dihydro-2H-[1,4]oxazin-3- | | |
| | yl)-4-fluoro-phenyl]-amide hydrochloride | | |
| 238 | 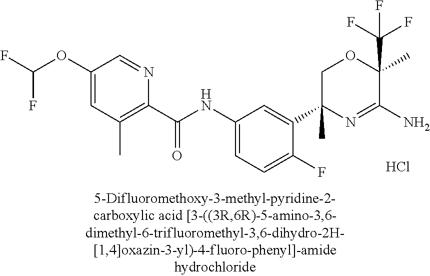 [SMILES: Cc1cc(OC(F)F)cnc1C(=O)Nc1ccc(F)c([C@]2(C)CO[C@@](C)(C(F)(F)F)C(N)=N2)c1 ; Cl] | 11.53 (br s, 1H), 10.78 (s, 1H), 9.54 (br s, 2H), 8.45 (d, 1H), 8.00-7.94 (m, 1H), 7.88 (dd, 1H), 7.75 (d, 1H), 7.45 (t, 1H), 7.32 (dd, 1H), 4.32 (d, 1H), 4.08 (d, 1H), 2.60 (s, 3H), 1.75 (s, 3H), 1.72 (s, 3H). | 491 |
| 239 | 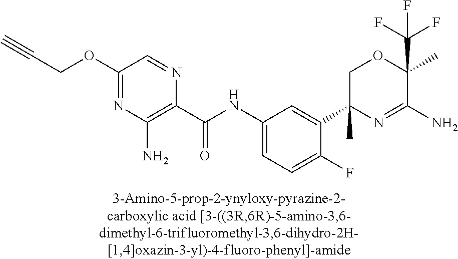 [SMILES: C#CCOc1cnc(C(=O)Nc2ccc(F)c([C@]3(C)CO[C@@](C)(C(F)(F)F)C(N)=N3)c2)c(N)n1] | 10.20 (s, 1H), 8.05 (br. s, 1H), 7.85 (dd, 1H), 7.70-7.61 (m, 1H), 7.57 (s, 1H), 7.52 (br. s, 1H), 7.11 (dd, 1H), 6.07 (br. s, 2H), 5.01 (d, 2H), 3.94-3.84 (m, 1H), 3.80 (d, 1H), 3.63 (s, 1H), 1.48 (s, 3H), 1.40 (s, 3H). | 481 |
| 240 | 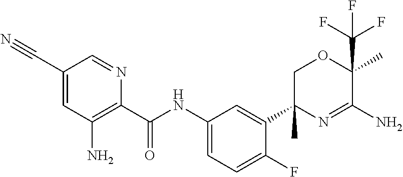 [SMILES: C[C@@]1(c2cc(NC(=O)c3ncc(C#N)cc3N)ccc2F)CO[C@@](C)(C(F)(F)F)C(N)=N1] | 10.54 (s, 1H), 8.21 (d, 1H), 7.85 (dd, 1H), 7.70 (d, 1H), 7.64 (d, 1H), 7.25 (br. s, 2H), 7.13 (dd, 1H), 6.06 (br. s, 2H), 3.87 (d, 1H), 3.82 (d, 1H), 1.48 (s, 3H), 1.40 (s, 3H). | 451 |
| | 3-Amino-5-cyano-pyridine-2-carboxylic acid | | |
| | [3-((3R,6R)-5-amino-3,6-dimethyl-6- | | |
| | trifluoromethyl-3,6-dihydro-2H-[1,4]oxazin-3- | | |
| | yl)-4-fluoro-phenyl]-amide | | |
| 241 | 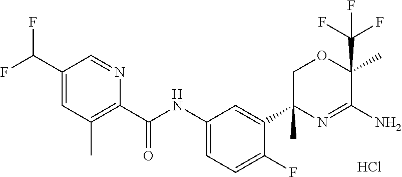 [SMILES: Cc1cc(C(F)F)cnc1C(=O)Nc1ccc(F)c([C@]2(C)CO[C@@](C)(C(F)(F)F)C(N)=N2)c1 ; Cl] | 11.57 (br s, 1H), 10.88 (s, 1H), 9.56 (br s, 2H), 8.74 (s, 1H), 8.07 (s, 1H), 8.00-7.93 (m, 1H), 7.88 (dd, 1H), 7.40- 7.08 (m, 2H), 4.32 (d, 1H), 4.08 (d, 1H), 2.60 (s, 3H), 1.75 (s, 3H), 1.73 (s, 3H). | 475 |
| | 5-Difluoromethyl-3-methyl-pyridine-2- | | |
| | carboxylic acid [3-((3R,6R)-5-amino-3,6- | | |
| | dimethyl-6-trifluoromethyl-3,6-dihydro-2H- | | |
| | [1,4]oxazin-3-yl)-4-fluoro-phenyl]-amide | | |
| | hydrochloride | | |
| 242 | 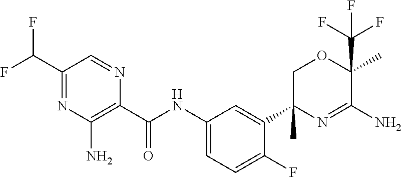 [SMILES: C[C@@]1(c2cc(NC(=O)c3ncc(C(F)F)nc3N)ccc2F)CO[C@@](C)(C(F)(F)F)C(N)=N1] | 10.68 (s, 1H), 8.13 (s, 1H), 7.98-7.81 (m, 3H), 7.67 (d, 1H), 7.14 (dd, 1H), 6.95 (t, 1H, CHF2), 6.07 (br. s, 2H), 3.89 (d, 1H), 3.80 (d, 1H), 1.48 (s, 3H), 1.40 (s, 3H). | 477 |
| | 3-Amino-5-difluoromethyl-pyrazine-2- | | |
| | carboxylic acid [3-((3R,6R)-5-amino-3,6- | | |
| | dimethyl-6-trifluoromethyl-3,6-dihydro-2H- | | |
| | [1,4]oxazin-3-yl)-4-fluoro-phenyl]-amide | | |
| 243 | 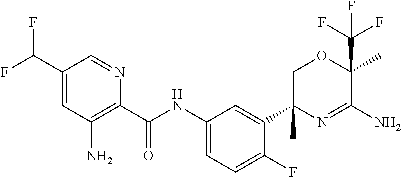 [SMILES: C[C@@]1(c2cc(NC(=O)c3ncc(C(F)F)cc3N)ccc2F)CO[C@@](C)(C(F)(F)F)C(N)=N1] | 10.48 (s, 1H), 8.03 (s, 1H), 7.86 (dd, 1H), 7.71 (d, 1H), 7.40 (s, 1H), 7.19-7.11 (m, 3 H), 7.12 (t, 1H, CHF2), 6.06 (br. s, 2H), 3.93-3.74 (m, 2H), 1.49 (s, 3H), 1.41 (s, 3H). | 476 |
| | 3-Amino-5-difluoromethyl-pyridine-2- | | |
| | carboxylic acid [3-((3R,6R)-5-amino-3,6- | | |
| | dimethyl-6-trifluoromethyl-3,6-dihydro-2H- | | |
| | [1,4]oxazin-3-yl)-4-fluoro-phenyl]-amide | | |
| 244 | 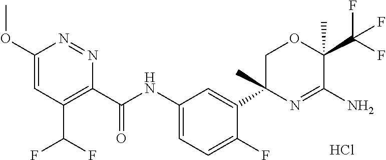 [SMILES: COc1cc(C(F)F)c(C(=O)Nc2ccc(F)c([C@]3(C)CO[C@@](C)(C(F)(F)F)C(N)=N3)c2)nn1 ; Cl] | 11.53 (s, 1H), 11.31 (s, 1H), 9.55 (s, 2H), 8.03 (d, 1H), 7.91 (dd, 1H), 7.66 (s, 1H), 7.61 (t, 1H), 7.36 (dd, 1H), 4.32 (d, 1H), 4.20 (s, 3H), 4.08 (d, 1H), 1.75 (s, 3H), 1.7 (s, 3H). | 492 |
| | 4-Difluoromethyl-6-methoxy-pyridazine-3- | | |
| | carboxylic acid [3-((3R,6R)-5-amino-3,6- | | |
| | dimethyl-6-trifluoromethyl-3,6-dihydro-2H- | | |
| | [1,4]oxazin-3-yl)-4-fluoro-phenyl]-amide | | |
| | hydrochloride | | |
| 245 | 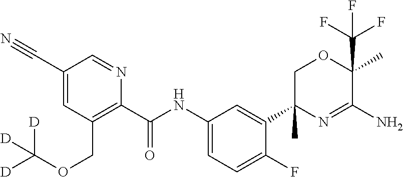 [SMILES: [2H]C([2H])([2H])OCc1cc(C#N)cnc1C(=O)Nc1ccc(F)c([C@]2(C)CO[C@@](C)(C(F)(F)F)C(N)=N2)c1] | 10.8 (s, 1H, NH), 9.08 (s, 1H), 8.47 (s, 1H), 7.81 (m, 1H), 7.73 (s broad, 1H), 7.16 (triplettoid, 1H), 6.08 (s broad, 2H, NH2 amidine), 4.81 (s, 2H), 3.90 (d, 1H, AB), 3.80 (d, 1H, AB), 1.45 (s, 3H), 1.40 (s, 3H). | 483 |
| | 5-Cyano-3-trideuteromethoxymethyl-pyridine- | | |
| | 2-carboxylic acid [3-((3R,6R)-5-amino-3,6- | | |
| | dimethyl-6-trifluoromethyl-3,6-dihydro-2H- | | |
| | [1,4]oxazin-3-yl)-4-fluoro-phenyl]-amide | | |
| 246 | 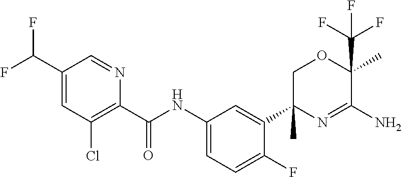 [SMILES: C[C@@]1(c2cc(NC(=O)c3ncc(C(F)F)cc3Cl)ccc2F)CO[C@@](C)(C(F)(F)F)C(N)=N1] | 10.83 (s, 1H), 8.86 (s, 1H), 8.40 (s, 1H), 7.74-7.70 (m, 2H), 7.25 (t, 1H, CHF2), 7.20-7.16 (m, 1H), 6.11 (br. s, 2H), 3.95- 3.93 (m, 1H), 3.80- 3.78 (m, 1H), 1.47 (s, 3 H), 1.43 (s, 3H). | 495 |
| | 3-Chloro-5-difluormethyl-pyridine-2-carboxylic | | |
| | acid [3-((3R,6R)-5-amino-3,6-dimethyl-6- | | |
| | trifluoromethyl-3,6-dihydro-2H-[1,4]oxazin-3- | | |
| | yl)-4-fluoro-phenyl]-amide-amide | | |
| 247 | 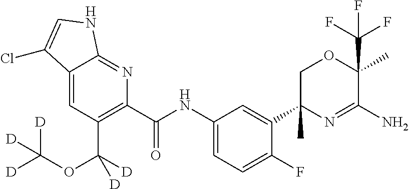 [SMILES: [2H]C([2H])([2H])OC([2H])([2H])c1cc2c(Cl)c[nH]c2nc1C(=O)Nc1ccc(F)c([C@]2(C)CO[C@@](C)(C(F)(F)F)C(N)=N2)c1] | 12.3 (s, 1H, NH), 11.65 (s, 1H, ⁺N—H, amidine), 10.72 (s, 1H, NH, amide), 9.82 (s, NH, amidine), 9.48 (s, 1H, NH, amidine), 8.12 (s, 1H), 7.90 (m, 3H), 7.31 (dd, 1H), 4.32 (d, 1H, AB), 4.08 (d, 1H, AB), 1.75 (s, 3H), 1.72 (s, 3H). | 533 |
| | 3-Chloro-5-trideuteromethoxy- | | |
| | dideuteromethyl-1H-pyrrolo[2,3-b]pyridine-6- | | |
| | carboxylic acid [3-((3R,6R)-5-amino-3,6- | | |
| | dimethyl-6-trifluoromethyl-3,6-dihydro-2H- | | |
| | [1,4]oxazin-3-yl)-4-fluoro-phenyl]-amide | | |
| 248 | 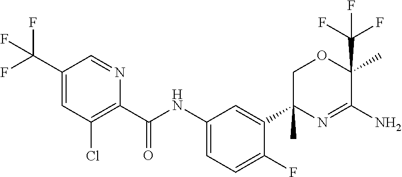 [SMILES: C[C@@]1(c2cc(NC(=O)c3ncc(C(F)(F)F)cc3Cl)ccc2F)CO[C@@](C)(C(F)(F)F)C(N)=N1] | 10.88 (s, 1H), 9.08 (s, 1H), 8.72 (s, 1H), 7.71 (dd, 2H), 7.21- 7.17 (m, 1H), 6.11 (br. s, 2H), 3.94 (d, 1H), 3.79 (d, 1H), 1.46 (s, 3H), 1.42 (s, 3H). | 513 |
| | 3-Chloro-5-trifluoromethyl-pyridine-2- | | |
| | carboxylic acid [3-((3R,6R)-5-amino-3,6- | | |
| | dimethyl-6-trifluoromethyl-3,6-dihydro-2H- | | |
| | [1,4]oxazin-3-yl)-4-fluoro-phenyl]-amide | | |
| 249 | 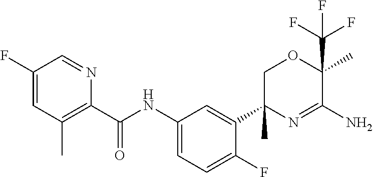 [SMILES: Cc1cc(F)cnc1C(=O)Nc1ccc(F)c([C@]2(C)CO[C@@](C)(C(F)(F)F)C(N)=N2)c1] | 10.50 (br s, 1H), 8.53 (d, 1H), 7.84-7.78 (m, 2H), 7.73 (br s, 1H), 7.14 (dd, 1H), 6.04 (br. s, 2H), 3.91 (d, 1H), 3.80 (d, 1H), 2.57 (s, 3H), 1.48 (br s, 3H), 1.42 (br s, 3H) | 443 |
| | 5-Fluoro-3-methyl-pyridine-2-carboxylic acid | | |
| | [3-((3R,6R)-5-amino-3,6-dimethyl-6-trifluoro- | | |
| | methyl-3,6-dihydro-2H-[1,4]oxazin-3-yl)-4- | | |
| | fluoro-phenyl]-amide | | |
| 250 | 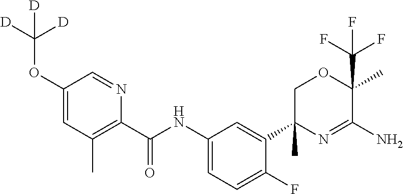 [SMILES: [2H]C([2H])([2H])Oc1cnc(C(=O)Nc2ccc(F)c([C@]3(C)CO[C@@](C)(C(F)(F)F)C(N)=N3)c2)c(C)c1] | 10.37 (br s, 1H), 8.22 (d, 1H), 7.81 (dd, 1H), 7.78-7.71 (m, 1H), 7.40 (d,1H), 7.12 (dd, 1H), 6.04 (br. s, 2H), 3.89 (d, 1H), 3.82 (d, 1H), 2.61 (s, 3H), 1.49 (br s, 3H), 1.42 (br s, 3H) | 458 |
| | 5-Trideuteromethoxy-3-methyl-pyridine-2- | | |
| | carboxylic acid [3-((3R,6R)-5-amino-3,6- | | |
| | dimethyl-6-trifluoromethyl-3,6-dihydro-2H- | | |
| | [1,4]oxazin-3-yl)-4-fluoro-phenyl]-amide | | |

### Example 251

### 5-Cyano-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide

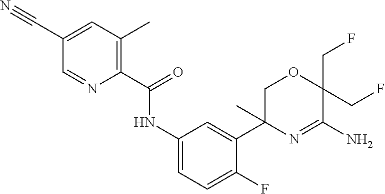 [SMILES: Cc1cc(C#N)cnc1C(=O)Nc1ccc(F)c(C2(C)COC(CF)(CF)C(N)=N2)c1]

### a) 3-Fluoro-2-fluoromethyl-2-trimethylsilanyloxy-propionitrile

To 1,3-Difluoro-propan-2-one (8.5 g, 90 mmol) was added drop wise over 30 min TMS-Cyanide (8.97 g, 90 mmol). The reaction mixture was stirred for 16 h at ambient temperature.

¹H-NMR (400 MHz, CDCl₃): δ 4.55 (d, 2H), 4.44 (d, 2H), 0.28 (s, 9H);

¹⁹F-NMR (376 MHz, CDCl₃): δ−226 (t).

### b) 3-Fluoro-2-fluoromethyl-2-hydroxy-propionic acid

3-Fluoro-2-fluoromethyl-2-trimethylsilanyloxy-propionitrile (17.4 g, 90 mmol) was treated with 37% HCl (300 ml) and heated to gentle reflux for 3 h. The reaction mixture was cooled to ambient temperature and concentrated in vacuo. The solid thus obtained was redisolved in 300 ml Ethanol and concentrated in vacuo and dried in high vacuum. The solid thus obtained (17 g) contained significant amount of Ammonium-Chloride and was used without further purification.

¹H-NMR (400 MHz, DMSO-D₆): δ 7.3-7.0 (m, 4H), 6.5-5.6 (s, 1H), 4.58-4.43 (m, 4H).

¹³C-NMR (150 MHz, DMSO-D₆): δ 171 (t), 85 (d), 83 (d), 75 (t).

### c) 3-Fluoro-2-fluoromethyl-2-hydroxy-propionic acid ethyl ester

Crude 3-Fluoro-2-fluoromethyl-2-hydroxy-propionic acid (17 g) was dissolved in Ethanol (400 ml) and H₂SO₄ (98%, 30 g) was added. The reaction mixture was refluxed for 16 h. The reaction mixture was cooled to ambient temperature and filtered. The solution was carefully treated with 30 g solid Na₂CO₃ and the resulting mixture was stirred for 30 min at room temperature. 400 ml DCM were added and the mixture was filtered. The solution was concentrated (50° C., 150 mbar) and further purified by distillation (82° C., 20 mbar) to give a colorless liquid.

¹H-NMR (400 MHz, DMSO-D₆): δ 4.65-4.43 (m, 4H), 4.30 (q, 2H), 3.88-3.63 (s, 1H), 1.30 (t, 3H).

### d) 2-[2-(5-Bromo-2-fluoro-phenyl)-2-(2-nitro-benzenesulfonylamino)-propoxy]-3-fluoro-2-fluoromethyl-propionic acid ethyl ester

To a suspension of NaH (1.62 g, 60%, 40.5 mmol) in 75 ml DMF was added 3-Fluoro-2-fluoromethyl-2-hydroxy-propionic acid ethyl ester (6.8 g, 40.5 mmol). The reaction mixture was stirred at ambient temperature for 30 min and then rac. 2-(5-Bromo-2-fluoro-phenyl)-2-methyl-1-(2-nitro-benzenesulfonyl)-aziridine (14 g, 33.7 mmol, analogous to example 231 step a-h)) was added. The reaction mixture was stirred at ambient temperature for 2 days.

The reaction mixture was added to a cold solution of 2N aq. HCl (250 ml) and the product was extracted with 2×250 ml EtOAc., washed with NaHCO₃ solution (250 ml) and brine (250 ml). The organic layer was dried over MgSO₄ and concentrated under reduced pressure to obtain an off-white solid which was titruated with cold Methanol.

HPLC: Rt_{H3}=1.26 min; ESIMS [M+H₃O]⁺=600, 602;

¹H-NMR (400 MHz, DMSO): δ 8.40 (s, 1H), 7.89 (d, 1H), 7.85-7.60 (m, 3H), 7.45 (d, 1H), 6.91 (dd, 1H), 4.85-4.45 (m, 4H), 4.20 (q, 2H), 4.00 (d, 1H), 3.81 (d, 1H), 1.61 (s, 3H), 1.20 (t, 3H).

### e) 2-[2-(5-Bromo-2-fluoro-phenyl)-2-(2-nitro-benzenesulfonylamino)-propoxy]-3-fluoro-2-fluoromethyl-propionamide

2-[2-(5-Bromo-2-fluoro-phenyl)-2-(2-nitro-benzenesulfonylamino)-propoxy]-3-fluoro-2-fluoromethyl-propionic acid ethyl ester (10 g, 17.14 mmol) was dissolved in 7N NH₃ in MeOH (40 ml) and the yellow reaction mixture was stirred at 50-55° C. for 16 h in a sealed vial.

The reaction mixture was concentrated under reduced pressure to obtain a pale yellow solid.

HPLC: Rt_{H3}=1.05 min; ESIMS [M+H₃O]⁺=571, 573;

¹H-NMR (400 MHz, DMSO): δ 8.85-8.65 (s, 1H), 7.95-7.40 (m, 6H), 6.95 (m, 1H), 4.63 (d, 4H), 3.88 (m, 2H), 1.56 (s, 3H).

### f) N-[1-(5-Bromo-2-fluoro-phenyl)-2-(cyano-bis-fluoromethyl-methoxy)-1-methyl-ethyl]-2-nitro-benzenesulfonamide

2-[2-(5-Bromo-2-fluoro-phenyl)-2-(2-nitro-benzenesulfonylamino)-propoxy]-3-fluoro-2-fluoromethyl-propionamide (5 g, 9 mmol) was suspended in 150 ml dry DCM. N-Methyl-morpholine (2.5 ml) was added. TFAA (2.3 g, 10.8 mmol) was added in 20 ml DCM dropwise over 5 min. The reaction mixture was stirred at ambient temperature for 40 min.

N-Methyl-morpholine (2.5 ml) was added. TFAA (2.3 g, 10.8 mmol) was added in 20 ml DCM dropwise over 5 min.

The reaction mixture was added to a cold saturated aqueous solution of NaHCO₃ (400 ml) and the mixture was stirred for 5 min at RT. The phases were separated and the aqueous was extracted 2× with DCM (100 ml). The Combined organic phases were washed with cold 0.1 N HCl (100 ml), water (100 ml) and sat. NaHCO₃ solution (100 ml), dried over MgSO4, filtered and concentrated.

HPLC: Rt_{H3}=1.17 min; ESIMS [M+H₃O]⁺=553, 555;

¹H-NMR (400 MHz, CDCl₃): δ 7.89 (d, 1H), 7.70-7.47 (m, 4H), 7.31 (d, 1H), 6.59 (dd, 1H), 6.21 (s, 1H), 4.67 (m, 2H), 4.56 (m, 2H), 4.25 (d, 1H), 4.17 (d, 1H), 1.83 (s, 3H).

### g) 5-(5-Bromo-2-fluoro-phenyl)-2,2-bis-fluoromethyl-5-methyl-5,6-dihydro-2H-[1,4]oxazin-3-ylamine

A suspension of N-[1-(5-Bromo-2-fluoro-phenyl)-2-(cyano-bis-fluoromethyl-methoxy)-1-methyl-ethyl]-2-nitro-benzenesulfonamide (5 g, 9.32 mmol), K2CO3 (2.83 g, 20.51 mmol) and 2-Acetylamino-3-mercapto-propionic acid (3.8 g, 23.31 mmol) in EtOH (100 ml) was refluxed for 16 h. The reaction mixture was cooled to RT and filtered. The solution was concentrated to obtain a yellow solid foam.

The solid foam was suspended in 10% Na₂CO₃ solution (50 ml) and was extracted with EtOAc (3×200 ml). The combined organic layers were washed with 10% aq. Na₂CO₃ solution (50 ml), 1 M NaOH (50 ml) and brine (50 ml). The solution was dried over MgSO4, filtered and evaporated.

HPLC: Rt_{H3}=1.17 min; ESIMS [M+H]⁺=351, 353;

¹H-NMR (400 MHz, CDCl₃): δ 7.68 (dd, 1H), 7.33 (m, 1H), 6.89 (dd, 1H), 4.75-4.39 (m, 4H), 3.96 (d, 1H), 3.87 (d, 1H), 1.51 (s, 3H).

### h) [5-(5-Bromo-2-fluoro-phenyl)-2,2-bis-fluoromethyl-5-methyl-5,6-dihydro-2H-[1,4]oxazin-3-yl]-carbamic acid tert-butyl ester

5-(5-Bromo-2-fluoro-phenyl)-2,2-bis-fluoromethyl-5-methyl-5,6-dihydro-2H-[1,4]oxazin-3-ylamine (3.3 g, 9.4 mmol) was dissolved in 100 ml DCM. Boc-Anhydride (2.46 g, 11.48 mmol) was added and the reaction mixture was stirred at ambient temperature for 16 h. The reaction mixture was treated with 10% aqueous Citric acid (50 ml) solution and stirred for 5 min at RT. The phases were separated and the organic layers were washed with NaHCO₃ solution (25 ml) and brine (25 ml). The solution was dried over MgSO4, filtered and evaporated. The crude product was purified via silica-gel chromatography to provide the title compound as a white crystalline solid. TLC (Hexane/EtOAc 9:1):

R_{f}=0.27;

HPLC: Rt_{H3}=1.27 min; ESIMS [M+H]⁺=451, 453;

¹H-NMR (400 MHz, CDCl₃): δ 10.98-10.94 (s, 1H), 7.43 (m, 2H), 6.98 (m, 1H), 5.04-4.88 (dd, 1H), 4.77-4.70 (m, 1H), 4.63-4.43 (m, 3H), 4.06 (d, 1H), 1.67 (s, 3H), 1.53 (s, 9H).

### i) [5-(5-Amino-2-fluoro-phenyl)-2,2-bis-fluoromethyl-5-methyl-5,6-dihydro-2H-[1,4]oxazin-3-yl]-carbamic acid tert-butyl ester

[5-(5-Bromo-2-fluoro-phenyl)-2,2-bis-fluoromethyl-5-methyl-5,6-dihydro-2H-[1,4]oxazin-3-yl]-carbamic acid tert-butyl ester (2.27 g, 5.03 mmol), rac-trans-N,N-Dimethylcyclohexane-1,2-diamine (715 mg, 5.03 mmol), Sodium ascorbate (400 mg, 2 mmol), NaN₃ (2.62, 40.2 mmol) were suspended in EtOH (100 ml) and H₂O (43 ml). The reaction mixture was degassed and CuI (383 mg, 2 mmol) were added under N₂. The reaction mixture was stirred at 70° C. for 45 min. The reaction mixture was cooled to RT and water (100 ml) and EtOAc (200 ml) were added. The phases were separated and the aqueous phase was extracted with EtOAc (200 ml). The combined organic phases were washed with water (250 ml), 5% aqueous Ammonia (250 ml) and brine (250 ml). The organic layer was dried over anhydrous Na₂SO₄ and the organic layer was concentrated under reduced pressure. The solid obtained was dissolved in Ethanol (50 ml) and Pd/C 5% (350 mg, E101 N/D Degussa) was added. The reaction mixture was degassed and hydrogenated at 1.1 bar for 1 h at ambient temperature. The reaction mixture was filtered and concentrated. The crude product was purified via silica-gel chromatography (gradient: Hexane/EtOAc 6%→Hexane/EtOAc 48%) to provide the title compound as a white crystalline solid: TLC (Hexane/EtOAc 2:1): Rf=0.66; HPLC: Rt_{H3}=1.07 min; ESIMS [M+H]⁺=388;

¹H-NMR (400 MHz, DMSO): δ 10.75-10.72 (s, 1H), 6.90 (m, 1H), 6.50 (m, 2H), 5.10-5.03 (s, 1H), 5.04-4.40 (m, 4H), 4.32-4.05 (dd, 2H), 1.60 (s, 3H), 1.42 (s, 9H).

### j) (5-{5-[(5-Cyano-3-methyl-pyridine-2-carbonyl)-amino]-2-fluoro-phenyl}-2,2-bis-fluoromethyl-5-methyl-5,6-dihydro-2H-[1,4]oxazin-3-yl)-carbamic acid tert-butyl ester

[5-(5-Amino-2-fluoro-phenyl)-2,2-bis-fluoromethyl-5-methyl-5,6-dihydro-2H-[1,4]oxazin-3-yl]-carbamic acid tert-butyl ester (400 mg, 1.03 mmol), 5-cyano-3-methyl-pyridine-2-carboxylic acid (201 mg, 1.24 mmol), HOAT (215, 1.55 mmol) and N-Methyl-morpholine (209 mg, 2.65 mmol) were dissolved in dry DMF (10 ml). EDC*HCl (297 mg, 1.55 mmol) were added and the reaction mixture was stirred at ambient temperature for 3 h. The reaction mixture was treated with water (30 ml) and EtOAc (50 ml and stirred for 5 min at RT. The phases were separated and the organic layers were washed with NaHCO₃ solution (25 ml) and brine (25 ml). The solution was dried over Na₂SO₄, filtered and evaporated. The crude product was purified via silica-gel chromatography to provide the title compound as a white crystalline solid. TLC (Hexane/EtOAc 7:3): Rf=0.39; HPLC: Rt_{H3}=1.24 min; ESIMS [M+H]⁺=532;

¹H-NMR (400 MHz, CDCl₃): δ 11.09-11.01 (s, 1H), 10.01 (s, 1H), 8.71 (s, 1H), 7.93 (s, 1H), 7.80 (m, 1H), 7.58 (m, 1H), 7.12 (m, 1H), 5.06-4.90 (dd, 1H), 4.76 (d, 1H), 4.67-4.45 (m, 3H), 4.10 (m, 1H), 2.83 (s, 3H), 1.72 (s, 3H), 1.54 (s, 9H).

### k) 5-Cyano-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide

(5-{5-[(5-Cyano-3-methyl-pyridine-2-carbonyl)-amino]-2-fluoro-phenyl}-2,2-bis-fluoromethyl-5-methyl-5,6-dihydro-2H-[1,4]oxazin-3-yl)-carbamic acid tert-butyl ester (450 mg, 0.847 mmol) was dissolved in DCM (8 ml). TFA (965, 8.47 mmol) was added dropwise. The reaction mixture was then stirred 2 h at ambient temperature. The reaction mixture was added to a cold aqueous Na₂CO₃ solution (50 ml). DCM (30 ml) was added and the reaction mixture was stirred for 10 min. The phases were separated and the organic layers were washed with NaHCO₃ solution (25 ml) and brine (25 ml). The solution was dried over Na₂SO₄, filtered and evaporated to provide the title compound as a white solid.

HPLC: Rt_{H3}=0.73 min; ESIMS [M+H]⁺=432;

¹H-NMR (400 MHz, DMSO): δ 10.70-10.63 (br. s, 1H), 8.96 (s, 1H), 8.37 (s, 1H), 7.75 (m, 2H), 7.11 (m, 1H), 6.10-6.00 (s, 2H), 4.90 (dd, 1H), 4.73-4.47 (m, 3H), 3.85 (dd, 2H), 2.51 (s, 3H), 1.40 (s, 3H).

### Example 252

### 5-Cyano-3-methyl-pyridine-2-carboxylic acid [3-((R)-5-amino-6,6-bis-fluoromethyl-3-methyl-3,6-dihydro-2H-[1,4]oxazin-3-yl)-4-fluoro-phenyl]-amide

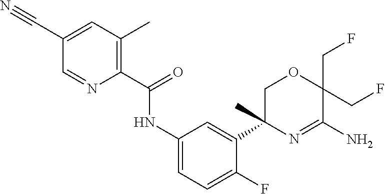 [SMILES: Cc1cc(C#N)cnc1C(=O)Nc1ccc(F)c([C@]2(C)COC(CF)(CF)C(N)=N2)c1]

The racemic product 5-Cyano-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide (350 mg) was separated via prep-HPLC on Chiralpak AD-H 320×7.65 mm column using n-Heptane/iPrOH 70:30 (+0.05% Diethyl Amine) as eluent.

The desired compound was the slower eluting (R)-enantiomer (146 mg, white solid, ee=100% (Detection at 210 nm)).

### Examples 253 to 255

The compounds listed in Table 25 were prepared by a procedure analogous to those used in Example 251 and Example 252.

TABLE 25

| | | | MS |
|---|---|---|---|
| | | ¹H-NMR | [m/z; |
| Example | Compound | (δ; DMSO-d₆) | (M + 1)⁺] |
| 253 | 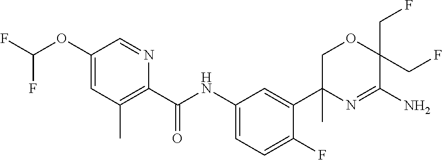 [SMILES: Cc1cc(OC(F)F)cnc1C(=O)Nc1ccc(F)c(C2(C)COC(CF)(CF)C(N)=N2)c1] | 10.43-10.47 (s, 1H), 8.41 (d, 1H), 7.68- 7.82 (m, 3H), 7.23- 7.60 (t, 1H), 7.10 (m, 1H), 6.00-6.10 (s, 2H), 4.45-5.00 (m, 4H), 3.87 (m, 2H), 2.57 (s, 3H), 1.42 (s, 3H). | 473 |
| | 5-Difluoromethoxy-3-methyl-pyridine-2- | | |
| | carboxylic acid [3-(5-amino-6,6-bis- | | |
| | fluoromethyl-3-methyl-3,6-dihydro-2H- | | |
| | [1,4]oxazin-3-yl)-4-fluoro-phenyl]-amide | | |
| 254 | 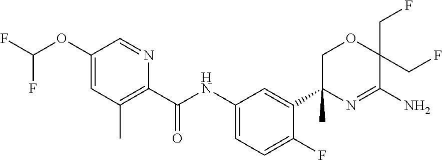 [SMILES: Cc1cc(OC(F)F)cnc1C(=O)Nc1ccc(F)c([C@]2(C)COC(CF)(CF)C(N)=N2)c1] | 10.43-10.47 (s, 1H), 8.41 (d, 1H), 7.68- 7.82 (m, 3H), 7.23- 7.60 (t, 1H), 7.10 (m, 1H), 6.00-6.10 (s, 2H), 4.45-5.00 (m, 4H), 3.87 (m, 2H), 2.57 (s, 3H), 1.42 (s, 3H). | 473 |
| | 5-Difluoromethoxy-3-methyl-pyridine-2- | | |
| | carboxylic acid [3-((R)-5-amino-6,6-bis- | | |
| | fluoromethyl-3-methyl-3,6-dihydro-2H- | | |
| | [1,4]oxazin-3-yl)-4-fluoro-phenyl]-amide | | |
| 255 | 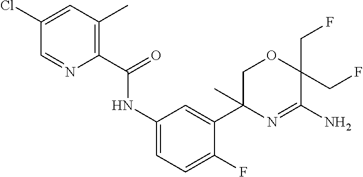 [SMILES: Cc1cc(Cl)cnc1C(=O)Nc1ccc(F)c(C2(C)COC(CF)(CF)C(N)=N2)c1] | 10.47-10.52 (s, 1H), 8.57 (s, 1H), 8.02 (s, 1H), 7.81 (m, 1H), 7.73 (m, 1H), 7.11 (m, 1H), 6.03-6.10 (s, 2H), 4.87-5.00 (d, 1H), 4.50-4.73 (m, 3H), 3.87 (dd, 2H), 2.54 (s, 3H), 1.41 (s, 3H). | 441 |
| | 5-Chloro-3-methyl-pyridine-2-carboxylic acid | | |
| | [3-(5-amino-6,6-bis-fluoromethyl-3-methyl- | | |
| | 3,6-dihydro-2H-[1,4]oxazin-3-yl)-4-fluoro- | | |
| | phenyl]-amide | | |

### Example 256

### 5-Cyano-3-methyl-pyridine-2-carboxylic acid [3-((R)-5-amino-3,6,6-tris-fluoromethyl-3,6-dihydro-2H-[1,4]oxazin-3-yl)-4-fluoro-phenyl]-amide

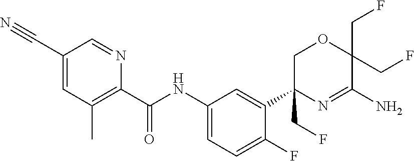 [SMILES: Cc1cc(C#N)cnc1C(=O)Nc1ccc(F)c([C@]2(CF)COC(CF)(CF)C(N)=N2)c1]

### a) (5-Bromo-2-fluoro-phenyl)-oxo-acetic acid ethyl ester

A solution of 22.80 ml (160 mmol) diisopropyl amine in 400 ml THF was cooled to −78° C. A 1.6 M solution of BuLi in hexanes (100 ml, 160 mmol) was added dropwise. After 15 minutes 25.45 g of 4-bromo-1-fluoro benzene (145 mmol) was added dropwise while keeping the temperature below −60° C. After stirring for 2.5 h at −70° C. 21.7 ml diethyl oxalate (160 mmol) were added. The mixture was warmed to −50° C. After 15 min the temperature cooled to −70° C. again, then the mixture was by poured onto 350 ml 1M HCl. The mixture was extracted with ligroin, dried with MgSO₄.H₂O, concentrated and distilled at ca 6 mbar (b.p. 112-115° C.) to give 31.58 g of the desired product as a yellow liquid. ¹H-NMR (CDCl₃, 400 MHz): δ 8.07 (dd, 1H), 7.77 (ddd, 1H), 7.12 (t, 1H), 4.47 (q, 2H), 1.44 (t, 3H).

### b) (R)-2-(5-Bromo-2-fluoro-phenyl)-2-hydroxy-3-nitro-propionic acid ethyl ester

To an at −25° C. cooled solution of 35.86 g (130 mmol) (5-bromo-2-fluoro-phenyl)-oxo-acetic acid ethyl ester and 3.59 g (6.52 mmol) Catalyst 1 (CHX135): 3,5-bis-trifluoromethyl-benzoic acid (R)-(6-hydroxy-quinolin-4-yl)-(5-vinyl-1-aza-bicyclo[2.2.2]oct-2-yl)-methyl ester (CAS registry: 1079392-85-0) in 360 ml DCM were added 70.3 ml (1.3 mol) nitromethane. The mixture was kept for 3 days at −20° C. till TLC analysis showed complete conversion. The catalyst was removed by passing the reaction mixture over a small pad of silica gel (DCM/(EtOH/sat aq NH₃ 9:1) 99:1). The crude product was purified by chromatography on silica gel (hexanes/EtOAc 5-15%) to give 39.88 g of the title compound as a colorless oil. E.e. 96%; HPLC: Rt_{H3}=2.705 min; ESIMS [M+Na]⁺=358/360(1Br); ¹H-NMR (CDCl₃, 400 MHz): δ 7.84 (dd, 1H), 7.51 (ddd, 1H), 7.01 (dd, 1H), 5.57 (d, 1H), 4.86 (d, 1H), 4.46-4.28 (m, 2H), 1.33 (t, 3H).

### c) (R)-3-Amino-2-(5-bromo-2-fluoro-phenyl)-2-hydroxy-propionic acid ethyl ester

Zn dust (78 g, 1.187 mol) was suspended in 240 ml AcOH using a mechanical stirrer. A solution of (R)-2-(5-bromo-2-fluoro-phenyl)-2-hydroxy-3-nitro-propionic acid ethyl ester (39.88 g, 119 mmol) in 160 ml AcOH was added dropwise to this suspension while keeping the temperature between 30-40° C. with the use of a water bath. After 15 min the mixture was filtered over celite and washed with EtOAc. The filtrate was concentrated, taken up in EtOAc and was washed with 10% soda solution. Any insoluble parts were dissolved by adding some aq. NH₃. The organic layer was washed with sat aq NaHCO₃ and brine, and dried with Na₂SO₄. Evaporation gave the 34 g of the title compound as a white solid, pure enough for further synthesis. HPLC: Rt_{H2}=2.397 min; ESIMS [M+H]⁺=306/308(1Br); ¹H-NMR (DMSO-d6, 400 MHz): δ 7.74 (dd, 1H), 7.54 (ddd, 1H), 7.14 (dd, 1H), 4.17-4.03 (m, 2H), 3.21 (d, 1H), 2.87 (d, 1H), 1.13 (t, 3H).

### d) (R)-3-Amino-2-(5-bromo-2-fluoro-phenyl)-propane-1,2-diol, hydrochloride

Under nitrogen atmosphere is added dropwise 1.415 ml BH₃.SMe₂ (neat, 14.9 mmol) to a solution of (R)-3-amino-2-(5-bromo-2-fluoro-phenyl)-2-hydroxy-propionic acid ethyl ester (1.52 g, 4.97 mmol) in 15 ml THF. The reaction is exothermic under evolution of gas. The mixture is heated to reflux for 3 h. The excess borane is quenched by the careful addition of 3 ml MeOH. More MeOH is added followed by 3 ml 2M aq. HCl. The mixture is evaporated, dissolved in 20 ml MeOH and evaporated (2×). The residue is crystallized from EtOH(EtOAc to give 907 mg of the title compound as white crystals. HPLC: Rt_{H1}=2.451 min; ESIMS [M+H]⁺=264/266(1Br); ¹H-NMR (DMSO-d6, 400 MHz): δ 7.80 (br, 2H), 7.76 (dd, 1H), 7.58 (ddd, 1H), 7.19 (dd, 1H), 6.30 (s, 1H), 5.28 (br s, 1H), 3.72 (d, 1H), 3.63 (d, 1H), 3.26 (d, 1H), 3.14 (d, 1H).

### e) N-[(R)-2-(5-Bromo-2-fluoro-phenyl)-2,3-dihydroxy-propyl]-2-nitro-benzenesulfonamide

A suspension of (R)-3-amino-2-(5-bromo-2-fluoro-phenyl)-propane-1,2-diol, hydrochloride (790 mg, 2.63 mmol), 2-nitro-benzenesulfonyl chloride (583 mg, 2.63 mmol), K₂CO₃ (363 mg, 2.63 mmol) and KHCO₃ (562 mg, 5.26 mmol) in 8 ml ACN was stirred for 2 h. The mixture was partitioned between EtOAc and brine. The organic layer was washed with brine, dried with MgSO₄.H₂O and evaporated. Chromatography on silica gel (hexanes/EtOAc 25-50%) gave 1.42 g of the title compound as a colorless foam. HPLC: Rt_{H2}=3.136 min; ESIMS [M+Na]⁺=371/373(1Br); ¹H-NMR (DMSO-d6, 400 MHz): δ 7.96-7.90 (m, 2H), 7.87-7.79 (m, 2H), 7.65 (dd, 1H), 7.58 (br, 1H), 7.44 (ddd, 1H), 7.03 (dd, 1H), 5.60 (s, 1H), 4.88 (t, 1H), 3.67-3.57 (m, 2H), 3.41 (d, 1H), 3.31 (d, 1H).

### f) (S)-2-(5-Bromo-2-fluoro-phenyl)-1-(2-nitro-benzenesulfonyl)-2-(tetrahydro-pyran-2-yloxymethyl)-aziridine

To an ice-cold solution of N—[(R)-2-(5-bromo-2-fluoro-phenyl)-2,3-dihydroxy-propyl]-2-nitro-benzenesulfonamide (1.40 g, 3.12 mmol) and dihydropyrane (0.299 ml, 3.27 mmol) in 14 ml DCM was added CSA (36 mg, 0.156 mmol). After warming to rt the mixture was stirred 2 h. EtOAc and sat. aq. NaHCO₃ were added and the organic phase was washed with brine, dried with MgSO₄.H₂O and evaporated. Chromatography on silica gel (hexanes/EtOAc 25-35%) gave 1.52 g of the title compound as a colorless resin. TLC (hexanes/EtOAc 2:1): Rf=0.28; HPLC: Rt_{H3}=3.348 min; ESIMS [M+Na]⁺=555/557(1Br).

This product was dissolved in 14 ml THF together with PPh3 (838 mg, 3.19 mmol), cooled to 0-5° C. and treated with a 40% toluene solution of DEAD (1.46 ml, 3.19 mmol) in a dropwise manner. Stirring was continued for 2.5 h while slowly warming to rt. The solution was diluted with 20 ml toluene, concentrated and directly purified via chromatography on silica gel (hexanes/EtOAc 5-15%) to give the title compound as a colorless resin (1:1 mixture of diastereomers). HPLC: Rt_{H4}=3.361 min; ESIMS [M+Na]⁺=537/539(1Br); ¹H-NMR (CDCl3, 400 MHz, 1:1 mixture of diastereomers): δ 8.32-8.27 (m, 1H), 7.81-7.76 (m, 3H), 7.71-7.65 (m, 1H), 7.46-7.42 (m, 1H), 6.95 (t, 1H), 5.74 and 5.62 (t, 1H), 4.36 and 4.34 (d, 1H), 4.12 and 4.10 (d, 1H), 3.74-3.57 (m, 1H), 3.52-3.44 (m, 1H), 3.52 and 3.35 (s, 1H), 2.99 and 2.94 (s, 1H).

### g) N—[(R)-1-(5-Bromo-2-fluoro-phenyl)-1-fluoromethyl-2-hydroxy-ethyl]-2-nitro-benzenesulfonamide

A mixture of (S)-2-(5-bromo-2-fluoro-phenyl)-1-(2-nitro-benzenesulfonyl)-2-(tetrahydro-pyran-2-yloxymethyl)-aziridine (1.08 g, 2.096 mmol) and TBAF.3H₂O (860 mg, 2.72 mmol) in 11 ml DMF was stirred overnight. The mixture was partitioned between brine and TBME. The organic layer was washed with diluted brine (3×), dried with MgSO₄.H₂O and evaporated to give 1.12 g of the mono fluoro THP ether as a yellow resin (1:1 mixture of diastereomers). TLC (hexanes/EtOAc 4:1): Rf=0.26; HPLC: Rt_{H4}=3.328 and 3.429 min; ESIMS [M+Na]⁺=557/559(1Br). The product was taken up in 16 ml MeOH and 6 ml THF containing 49 mg (0.209 mmol) CSA and stirred. After 6 h the reaction was complete and the homogeneous mixture was partitioned between EtOAc and sat aq NaHCO3. The organic phase was washed with sat. aq. NaHCO₃, dried with MgSO₄.H₂O and evaporated. The title compound was obtained as white crystals (741 mg, TBME/hexanes). HPLC: Rt_{H3}=2.733 min; ESIMS [M+Na]⁺=473/475(1Br); ¹H-NMR (DMSO-d6, 400 MHz): δ 8.40 (s, 1H), 7.88 (d, 1H), 7.77 (dt, 1H), 7.68-7.62 (m, 2H), 7.47-7.40 (m, 2H), 6.89 (dd, 1H), 5.38 (t, 1H), 5.07 (q, 1H), 4.94 (q, 1H), 3.89-3.73 (m, 2H).

### h) (R)-2-(5-Bromo-2-fluoro-phenyl)-2-fluoromethyl-1-(2-nitro-benzenesulfonyl)-aziridine

N—[(R)-1-(5-Bromo-2-fluoro-phenyl)-1-fluoromethyl-2-hydroxy-ethyl]-2-nitro-benzenesulfonamide (662 mg, 1.467 mmol) was dissolved in 7 ml THF together with PPh3 (462 mg, 1.76 mmol), cooled to 0-5° C. and treated with a 40% toluene solution of DEAD (0.807 ml, 1.76 mmol) in a dropwise manner. Stirring was continued for 2.5 h while slowly warming to rt. The solution was diluted with 20 ml toluene, concentrated and directly purified via chromatography on silica gel (hexanes/EtOAc 5-15%) to give the title compound as a colorless resin. HPLC: Rt_{H3}=3.274 min; ESIMS [M+Na]⁺=455/457(1Br); ¹H-NMR (CDCl3, 400 MHz): δ 8.34-8.30 (m, 1H), 7.84-7.80 (m, 3H), 7.68 (dd, 1H), 7.49 (ddd, 1H), 7.00 (t, 1H), 5.04 (d, 2H), 3.40 (s, 1H), 3.03 (d, 1H).

### i) 2-[(R)-2-(5-Bromo-2-fluoro-phenyl)-3-fluoro-2-(2-nitro-benzenesulfonylamino)-propoxy]-3-fluoro-2-fluoromethyl-propionic acid ethyl ester

To a suspension of NaH (78 mg, 60% in mineral oil, 1.94 mmol) in DMF (160 ml) was added drop-wise under argon 3-fluoro-2-fluoromethyl-2-hydroxy-propionic acid ethyl ester (327 mg, 1.94 mmol) and after stirring for 0.5 h at 20° C. (R)-2-(5-bromo-2-fluoro-phenyl)-2-fluoromethyl-1-(2-nitro-benzenesulfonyl)-aziridine (526 mg, 1.214 mmol). The reaction was kept at 25° C. for 16 h. The mixture was added to cold aq. 2N HCl and the product extracted with TBME. Combined organic layers were washed with saturated NaHCO₃ solution and brine, dried over MgSO₄.H₂O, filtered and concentrated. The residual compound was purified via chromatography on silica gel (hexanes/EtOAc 10-20%) to give the title compound as a white solid. TLC (hexanes/EtOAc 1:1): Rf=0.59; HPLC Rt_{H4}=3.230 min; ESIMS [M+Na]⁺=623, 625(1Br); ¹H NMR (400 MHz, CDCl₃): δ 7.93 (dd, 1H), 7.75 (dt, 1H), 7.66 (dt, 1H), 7.44 (dt, 1H), 7.39 (dd, 1H), 7.35 (ddd, 1H), 6.94 (s, 1H), 6.53 (dd, 1H), 5.33-4.62 (m, 6H), 4.39 (q, 2H), 4.19 (d, 1H), 4.14 (d, 1H), 1.37 (t, 3H).

### j) (R)-5-(5-Bromo-2-fluoro-phenyl)-2,2,5-tris-fluoromethyl-4-(2-nitro-benzenesulfonyl)-morpholin-3-one

To a solution of 2-[(R)-2-(5-bromo-2-fluoro-phenyl)-3-fluoro-2-(2-nitro-benzenesulfonylamino)-propoxy]-3-fluoro-2-fluoromethyl-propionic acid ethyl ester (462 mg, 0.768 mmol) in 3 ml MeOH and 2 ml THF were added 0.96 ml (3.84 mmol) of 4M aq. LiOH. The mixture was stirred at rt for 30 min. The reaction mixture was taken up in 1N HCl and EtOAc. The organic phase was washed with brine, dried with MgSO₄.H₂O and evaporated to give 445 mg of the acid as a white solid. HPLC Rt_{H4}=3.230 min; ESIMS [M+Na]⁺=595, 597(1Br). The acid was suspended in DCM and N-methyl morpholine (263 mg, 2.60 mmol) was added, followed by ethyl chloroformate (141 mg, 1.300 mmol) in a drop-wise manner. The resulting yellow solution was stirred at rt for 1 h. The reaction mixture was partitioned between 1N HCl and EtOAc. The organic layer was washed with brine and 10% aq NaHCO₃, dried with MgSO₄.H₂O and evaporated. Crystallization from TBME/hexanes provided the title compound. TLC (hexanes/EtOAc 3:1): Rf=0.20; HPLC Rt_{H4}=3.062 min; ESIMS [M+Na]⁺=577/579(1Br); ¹H NMR (400 MHz, CDCl₃): δ 8.30 (d, 1H), 7.83-7.74 (m, 4H), 7.57 (ddd, 1H), 7.08 (dd, 1H), 5.68 (dd, 1H), 5.47 (dd, 1H), 4.48 (ddd, 2H), 4.66 (dd, 1H), 4.60 (d, 1H), 4.51 (d, 1H), 4.39 (d, 1H).

### k) (R)-5-(5-Bromo-2-fluoro-phenyl)-2,2,5-tris-fluoromethyl-morpholin-3-one

A mixture of (R)-5-(5-bromo-2-fluoro-phenyl)-2,2,5-tris-fluoromethyl-4-(2-nitro-benzenesulfonyl)-morpholin-3-one (365 mg, 0.657 mmol), K₂CO₃ (363 mg, 2.63 mmol) and thioglycolic acid (121 mg, 1.315 mmol) in 3.5 ml DMF was stirred at 60° C. for 3 h. The mixture was diluted with EtOAc and brine. The org layer was washed with sat aq NaHCO₃ and brine, dried with MgSO₄.H₂O and evaporated. The residual compound was purified via chromatography on silica gel (hexanes/EtOAc 10-25%) to give the title compound as a white solid. TLC (hexanes/EtOAc 3:1): Rf=0.31; HPLC Rt_{H2}=3.202 min; ESIMS [M+H]⁺=370/372 (1×Br); ¹H NMR (400 MHz, CDCl₃): δ 7.56-7.51 (m, 2H), 7.06 (dd, 1H), 6.85 (br, 1H), 4.98-4.30 (m, 8H).

### l) (R)-5-(5-Bromo-2-fluoro-phenyl)-2,2,5-tris-fluoromethyl-morpholine-3-thione

To a solution of (R)-5-(5-bromo-2-fluoro-phenyl)-2,2,5-tris-fluoromethyl-morpholin-3-one (141 mg, 0.381 mmol) and hexamethyldisiloxane (111 mg, 0.686 mmol) in toluene was added phosphorous pentasulfide (102 mg, 0.457 mmol). The reaction mixture was heated to 100° C. and stirred 4 h. After the reaction mixture had been cooled to room temperature, 1 ml acetone and 1.42 ml aq K₂CO₃ solution (10% w/w) were added. This mixture was stirred for 90 minutes and then partitioned between water and EtOAc. The layers were separated, washed with 0.1 N NaOH, brine and EtOAc. The organic layers were combined, dried over MgSO₄.H₂O and evaporated. The crude product was purified via chromatography on silica gel (hexanes/EtOAc 10-15%) to give the title compound as a white solid: TLC (hexanes/EtOAc 6:1): Rf=0.38; HPLC Rt_{H2}=3.553 min; ESIMS [M+H]⁺=386/388 (1×Br); ¹H NMR (400 MHz, CDCl₃): δ 8.62 (br, 1H), 7.56 (ddd, 1H), 7.47 (dd, 1H), 7.08 (dd, 1H), 5.12-4.70 (m, 6H), 4.95 (d, 1H), 4.33 (d, 1H).

### m) (R)-5-(5-Bromo-2-fluoro-phenyl)-2,2,5-tris-fluoromethyl-5,6-dihydro-2H-[1,4]oxazin-3-ylamine

(R)-5-(5-Bromo-2-fluoro-phenyl)-2,2,5-tris-fluoromethyl-morpholine-3-thione (134 mg, 0.347 mmol) was dissolved in NH₃ solution 7 mol/l in methanol (3 ml). The sealed reaction vessel was heated to 80° C. for 3 days. The reaction mixture was evaporated and purified on a silica gel column by eluting with (hexanes/EtOAc 15-35%) to give the title compound as a colorless resin. TLC (hexanes/EtOAc 3:1): Rf=0.13; HPLC: Rt_{H2}=2.684 min; ESIMS [M+H]⁺=369/371(1Br); ¹H-NMR (CDCl3, 400 MHz): δ 11.91 (s, 1H), 7.72 (dd, 1H), 7.54-7.45 (m, 2H), 7.08-6.96 (m, 2H), 5.20-4.25 (m, 8H).

### n) [(R)-5-(5-Bromo-2-fluoro-phenyl)-2,2,5-tris-fluoromethyl-5,6-dihydro-2H-[1,4]oxazin-3-yl]-carbamic acid tert-butyl ester

To a solution of (R)-5-(5-bromo-2-fluoro-phenyl)-2,2,5-tris-fluoromethyl-5,6-dihydro-2H-[1,4]oxazin-3-ylamine (113 mg, 0.306 mmol) in 1 ml DCM were added DIPEA (60 mg, 0.46 mmol) and di-tert-butyldicarbonate (87 mg, 0.4 mmol). The reaction mixture was stirred overnight at 40° C. The reaction mixture was evaporated and purified on a silica gel column by eluting with hexanes/TBME 5-20% to give 132 mg of the title compound as a colorless foam. TLC (hexanes/EtOAc 9:1): Rf=0.16; HPLC: Rt_{H4}=3.123 min; ESIMS=[M+H]⁺469/471(1Br); ¹H-NMR (CDCl₃, 400 MHz): δ 11.22 (br s, 1H), 7.54-7.45 (m, 2H), 7.05 (dd, 1H), 5.06-4.34 (m, 8H), 1.53 (s, 9H).

### o) [(R)-5-(5-Amino-2-fluoro-phenyl)-2,2,5-tris-fluoromethyl-5,6-dihydro-2H-[1,4]oxazin-3-yl]-carbamic acid tert-butyl ester

To a solution [(R)-5-(5-bromo-2-fluoro-phenyl)-2,2,5-tris-fluoromethyl-5,6-dihydro-2H-[1,4]oxazin-3-yl]-carbamic acid tert-butyl ester (132 mg, 0.283 mmol) and 40.2 mg (0.283 mmol) trans-N,N′-dimethylcyclohexanes-1,2-diamine in 4 ml EtOH was added a solution of 147 mg (2.26 mmol) sodium azide and 22.4 mg (0.113 mmol) sodium-ascorbate in 1.6 ml water. The mixture was degassed and brought under nitrogen atmosphere. CuI (21.5 mg, 0.113 mmol) was added and the mixture was heated at 70° C. The initially formed suspension turned into a homogeneous blue solution. The mixture was cooled to rt, diluted with TBME and washed with diluted aq. NH₄OH and brine. The organic phase was dried with MgSO₄.H₂O and evaporated to give 128 mg of a yellow resin, consisting of a mixture of an azide intermediate and the title compound. The product was dissolved in 1.3 ml EtOH and 0.2 ml THF, treated with 68 mg 5% Pd—C “Degussa” E101 ND and stirred under an atmosphere of hydrogen until the starting material had been consumed. The mixture was diluted with DCM and filtered over Celite. The product was purified by chromatography on silica gel (hexanes/EtOAc 25-50%) to give 71 mg of the title compound as colorless foam. HPLC: Rt_{H2}=2.963 min; ESIMS=[M+H]⁺406; ¹H-NMR (CDCl₃, 400 MHz): δ 6.93 (dd, 1H), 6.72-6.67 (m, 2H), 5.09-4.33 (m, 8H), 1.53 (s, 9H).

### p) ((R)-5-{5-[(5-Cyano-3-methyl-pyridine-2-carbonyl)-amino]-2-fluoro-phenyl}-2,2,5-tris-fluoromethyl-5,6-dihydro-2H-[1,4]oxazin-3-yl)-carbamic acid tert-butyl ester

To an ice-cold solution of [(R)-5-(5-amino-2-fluoro-phenyl)-2,2,5-tris-fluoromethyl-5,6-dihydro-2H-[1,4]oxazin-3-yl]-carbamic acid tert-butyl ester (71 mg, 0.176 mmol), 5-cyano-3-methyl-pyridine-2-carboxylic acid (31.5 mg, 0.194 mmol), HOAt (38.4 mg, 0.282 mmol) in 0.72 ml DMF were added 0.04 ml (0.23 mmol) EDC (free base). The mixture was stirred at 0-5° C. for 1 h and 2 h at rt. EtOAc and water were added and the organic layer was washed with sat. aq. NaHCO₃, brine and dried with MgSO₄.H₂O. The product was purified by chromatography on silica gel (hexanes/EtOAc 15-50%) to give 94 mg of the title compound as colorless foam. TLC (hexane/EtOAc 3:1): Rf=0.18; HPLC: Rt_{H3}=3.452 min; ESIMS=[M+H]⁺550; ¹H-NMR (CDCl₃, 400 MHz): δ 11.28 (s, 1H), 10.12 (s, 1H), 8.76 (s, 1H), 7.99 (s, 1H), 7.92 (ddd, 1H), 7.71 (dd, 1H), 7.22 (dd, 1H), 5.05-4.44 (m, 8H), 2.89 (s, 3H), 1.59 (s, 9H).

### q) 5-Cyano-3-methyl-pyridine-2-carboxylic acid [3-((R)-5-amino-3,6,6-tris-fluoromethyl-3,6-dihydro-2H-[1,4]oxazin-3-yl)-4-fluoro-phenyl]-amide

To a solution of ((R)-5-{5-[(5-cyano-3-methyl-pyridine-2-carbonyl)-amino]-2-fluoro-phenyl}-2,2,5-tris-fluoromethyl-5,6-dihydro-2H-[1,4]oxazin-3-yl)-carbamic acid tert-butyl ester (94 mg, 0.172 mmol) in 0.75 ml DCM were added 0.25 ml TFA. The mixture was stirred for 1 h, poured onto 10% aq. Na2CO3 and extracted with EtOAc. The org layer was washed with brine and dried with Na₂SO₄. The product was purified by chromatography on silica gel (DCM/(EtOH/aq NH₃ 9:1) 0.5-1.5%) to give 59 mg of the title compound as colorless foam. HPLC: Rt_{H2}=2.850 min; ESIMS=[M+H]⁺450; ¹H-NMR (DMSO-d6, 600 MHz): δ 10.69 (s, 1H), 8.98 (s, 1H), 8.39 (s, 1H), 7.92 (m, 1H), 7.77 (m, 1H), 7.15 (dd, 1H), 6.33 (br s, 2H), 4.98-4.40 (m, 6H), 4.16 8d, 1H), 4.00 (d, 1H), 2.52 (s, 3H).

The examples in Table 26 below may also be made using the procedures described above or procedures analogous thereto.

TABLE 26

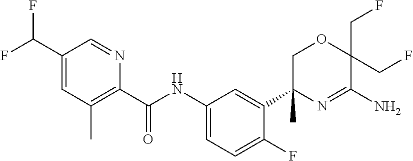 [SMILES: Cc1cc(C(F)F)cnc1C(=O)Nc1ccc(F)c([C@]2(C)COC(CF)(CF)C(N)=N2)c1]

5-Difluoromethyl-3-methyl-pyridine-2-
carboxylic acid [3-((R)-5-amino-6,6-bis-
fluoromethyl-3-methyl-3,6-dihydro-2H-
[1,4]oxazin-3-yl)-4-fluoro-phenyl]-amide

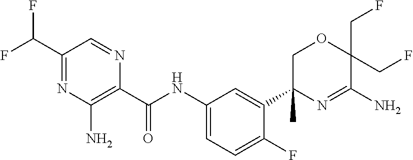 [SMILES: C[C@@]1(c2cc(NC(=O)c3ncc(C(F)F)nc3N)ccc2F)COC(CF)(CF)C(N)=N1]

3-Amino-5-difluoromethyl-pyrazine-2-
carboxylic acid [3-((R)-5-amino-6,6-bis-
fluoromethyl-3-methyl-3,6-dihydro-2H-
[1,4]oxazin-3-yl)-4-fluoro-phenyl]-amide

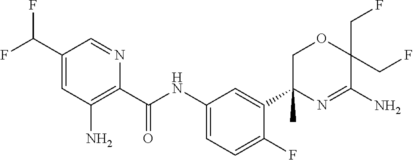 [SMILES: C[C@@]1(c2cc(NC(=O)c3ncc(C(F)F)cc3N)ccc2F)COC(CF)(CF)C(N)=N1]

3-Amino-5-difluoromethyl-pyridine-2-
carboxylic acid [3-((R)-5-amino-6,6-bis-
fluoromethyl-3-methyl-3,6-dihydro-2H-
[1,4]oxazin-3-yl)-4-fluoro-phenyl]-amide

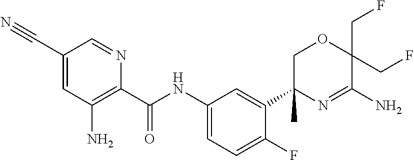 [SMILES: C[C@@]1(c2cc(NC(=O)c3ncc(C#N)cc3N)ccc2F)COC(CF)(CF)C(N)=N1]

3-Amino-5-cyano-pyridine-2-carboxylic acid
[3-((R)-5-amino-6,6-bis-fluoromethyl-3-
methyl-3,6-dihydro-2H-[1,4]oxazin-3-yl)-4-
fluoro-phenyl]-amide

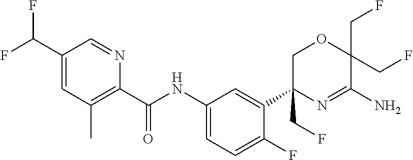 [SMILES: Cc1cc(C(F)F)cnc1C(=O)Nc1ccc(F)c([C@]2(CF)COC(CF)(CF)C(N)=N2)c1]

5-Difluoromethyl-3-methyl-pyridine-2-
carboxylic acid [3-((R)-5-amino-3,6,6-tris-
fluoromethyl-3,6-dihydro-2H-[1,4]oxazin-3-
yl)-4-fluoro-phenyl]-amide

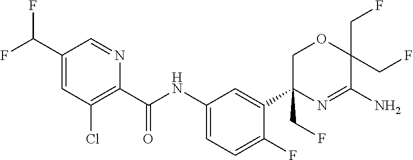 [SMILES: NC1=N[C@](CF)(c2cc(NC(=O)c3ncc(C(F)F)cc3Cl)ccc2F)COC1(CF)CF]

3-Chloro-5-difluoromethyl-pyridine-2-
carboxylic acid [3-((R)-5-amino-3,6,6-tris-
fluoromethyl-3,6-dihydro-2H-[1,4]oxazin-3-yl)-
4-fluoro-phenyl]-amide

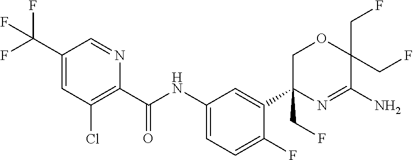 [SMILES: NC1=N[C@](CF)(c2cc(NC(=O)c3ncc(C(F)(F)F)cc3Cl)ccc2F)COC1(CF)CF]

3-Chloro-5-trifluoromethyl-pyridine-2-
carboxylic acid [3-((R)-5-amino-3,6,6-tris-
fluoromethyl-3,6-dihydro-2H-[1,4]oxazin-3-
yl)-4-fluoro-phenyl]-amide

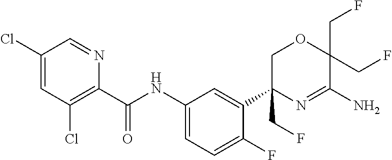 [SMILES: NC1=N[C@](CF)(c2cc(NC(=O)c3ncc(Cl)cc3Cl)ccc2F)COC1(CF)CF]

3,5-Dichloro-pyridine-2-carboxylic acid [3-
((R)-5-amino-3,6,6-tris-fluoromethyl-3,6-
dihydro-2H-[1,4]oxazin-3-yl)-4-fluoro-phenyl]-
amide

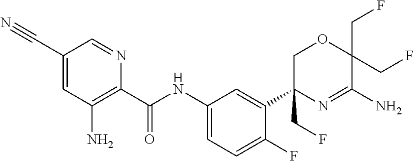 [SMILES: N#Cc1cnc(C(=O)Nc2ccc(F)c([C@]3(CF)COC(CF)(CF)C(N)=N3)c2)c(N)c1]

3-Amino-5-cyano-pyridine-2-carboxylic acid
[3-((R)-5-amino-3,6,6-tris-fluoromethyl-3,6-
dihydro-2H-[1,4]oxazin-3-yl)-4-fluoro-
phenyl]-amide

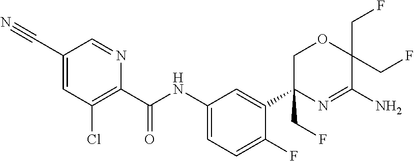 [SMILES: N#Cc1cnc(C(=O)Nc2ccc(F)c([C@]3(CF)COC(CF)(CF)C(N)=N3)c2)c(Cl)c1]

3-Chloro-5-cyano-pyridine-2-carboxylic acid
[3-((R)-5-amino-3,6,6-tris-fluoromethyl-3,6-
dihydro-2H-[1,4]oxazin-3-yl)-4-fluoro-phenyl]-
amide

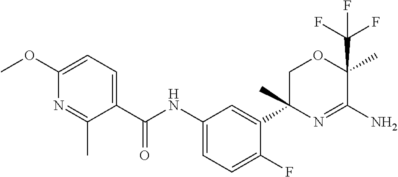 [SMILES: COc1ccc(C(=O)Nc2ccc(F)c([C@]3(C)CO[C@@](C)(C(F)(F)F)C(N)=N3)c2)c(C)n1]

N-[3-((3R,6R)-5-Amino-3,6-dimethyl-6-
trifluoromethyl-3,6-dihydro-2H-[1,4]oxazin-
3-yl)-4-fluoro-phenyl]-6-methoxy-2-methyl-
nicotinamide

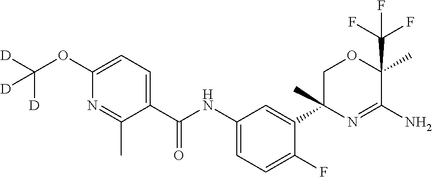 [SMILES: [2H]C([2H])([2H])Oc1ccc(C(=O)Nc2ccc(F)c([C@]3(C)CO[C@@](C)(C(F)(F)F)C(N)=N3)c2)c(C)n1]

N-[3-((3R,6R)-5-Amino-3,6-dimethyl-6-
trifluoromethyl-3,6-dihydro-2H-[1,4]oxazin-3-
yl)-4-fluoro-phenyl]-6-trideuteromethoxy-2-
methyl-nicotinamide

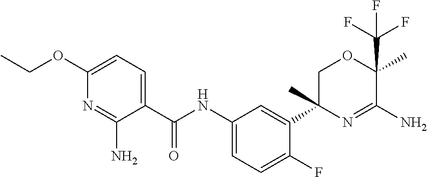 [SMILES: CCOc1ccc(C(=O)Nc2ccc(F)c([C@]3(C)CO[C@@](C)(C(F)(F)F)C(N)=N3)c2)c(N)n1]

2-Amino-N-[3-((3R,6R)-5-amino-3,6-
dimethyl-6-trifluoromethyl-3,6-dihydro-2H-
[1,4]oxazin-3-yl)-4-fluoro-phenyl]-6-ethoxy-
nicotinamide

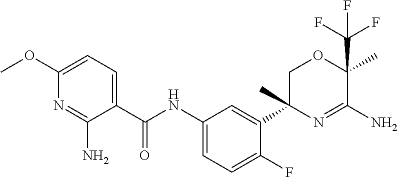 [SMILES: COc1ccc(C(=O)Nc2ccc(F)c([C@]3(C)CO[C@@](C)(C(F)(F)F)C(N)=N3)c2)c(N)n1]

2-Amino-N-[3-((3R,6R)-5-amino-3,6-dimethyl-
6-trifluoromethyl-3,6-dihydro-2H-[1,4]oxazin-
3-yl)-4-fluoro-phenyl]-6-methoxy-nicotinamide

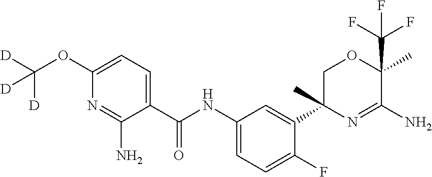 [SMILES: [2H]C([2H])([2H])Oc1ccc(C(=O)Nc2ccc(F)c([C@]3(C)CO[C@@](C)(C(F)(F)F)C(N)=N3)c2)c(N)n1]

2-Amino-N-[3-((3R,6R)-5-amino-3,6-
dimethyl-6-trifluoromethyl-3,6-dihydro-2H-
[1,4]oxazin-3-yl)-4-fluoro-phenyl]-6-
trideuteromethoxy-nicotinamide

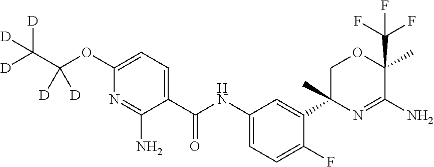 [SMILES: [2H]C([2H])([2H])C([2H])([2H])Oc1ccc(C(=O)Nc2ccc(F)c([C@]3(C)CO[C@@](C)(C(F)(F)F)C(N)=N3)c2)c(N)n1]

2-Amino-N-[3-((3R,6R)-5-amino-3,6-dimethyl-
6-trifluoromethyl-3,6-dihydro-2H-[1,4]oxazin-
3-yl)-4-fluoro-phenyl]-6-pentadeuteroethoxy-
nicotinamide

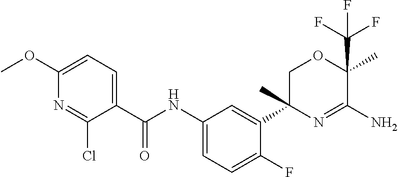 [SMILES: COc1ccc(C(=O)Nc2ccc(F)c([C@]3(C)CO[C@@](C)(C(F)(F)F)C(N)=N3)c2)c(Cl)n1]

N-[3-((3R,6R)-5-Amino-3,6-dimethyl-6-
trifluoromethyl-3,6-dihydro-2H-[1,4]oxazin-
3-yl)-4-fluoro-phenyl]-2-chloro-6-methoxy-
nicotinamide

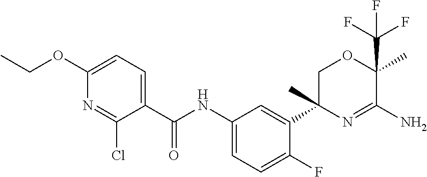 [SMILES: CCOc1ccc(C(=O)Nc2ccc(F)c([C@]3(C)CO[C@@](C)(C(F)(F)F)C(N)=N3)c2)c(Cl)n1]

N-[3-((3R,6R)-5-Amino-3,6-dimethyl-6-
trifluoromethyl-3,6-dihydro-2H-[1,4]oxazin-3-
yl)-4-fluoro-phenyl]-2-chloro-6-ethoxy-
nicotinamide

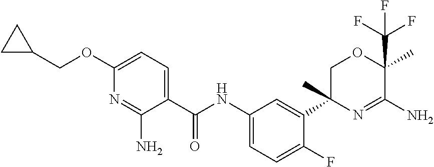 [SMILES: C[C@@]1(c2cc(NC(=O)c3ccc(OCC4CC4)nc3N)ccc2F)CO[C@@](C)(C(F)(F)F)C(N)=N1]

2-Amino-N-[3-((3R,6R)-5-amino-3,6-
dimethyl-6-trifluoromethyl-3,6-dihydro-2H-
[1,4]oxazin-3-yl)-4-fluoro-phenyl]-6-
cyclopropylmethoxy-nicotinamide

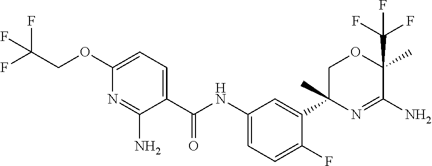 [SMILES: C[C@@]1(c2cc(NC(=O)c3ccc(OCC(F)(F)F)nc3N)ccc2F)CO[C@@](C)(C(F)(F)F)C(N)=N1]

2-Amino-N-[3-((3R,6R)-5-amino-3,6-dimethyl-
6-trifluoromethyl-3,6-dihydro-2H-[1,4]oxazin-
3-yl)-4-fluoro-phenyl]-6-(2,2,2-trifluoro-
ethoxy)-nicotinamide

### Example 257

### 5-Bromo-pyridine-2-carboxylic acid [6-((R)-5-amino-3-methyl-3,6-dihydro-2H-[1,4]-oxazin-3-yl)-pyridin-2-yl]-amide

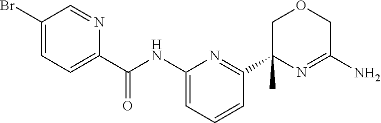 [SMILES: C[C@@]1(c2cccc(NC(=O)c3ccc(Br)cn3)n2)COCC(N)=N1]

### a) 5-(6-Bromo-pyridin-2-yl)-5-methyl-imidazolidine-2,4-dione

To a solution of 1-(6-bromo-pyridin-2-yl)ethanone (CAS 49669-13-8, 8.75 g, 43.7 mmol) and potassium cyanide (4.27 g, 65.6 mmol) in ethanol/water (40.0/26.7 ml) was added ammonium carbonate (21.02 g, 219.0 mmol). The reaction mixture was stirred in an autoclave at 100° C. for 17 h, then diluted with H₂O, 1M aq. NaHCO₃ soln. and EtOAc. The phases were separated and the aq. phase was reextracted with EtOAc, Et₂O and DCM. The combined org. phases were dried over Na₂SO₄, filtered and concentrated to leave the title compound as a pale white solid that was used in the next step without further purification.

HPLC Rt_{H11}=0.62 min; ESIMS: 270, 272 [(M+H)⁺]; ¹H NMR (400 MHz, DMSO-d₆): δ 10.86 (br s, 1H), 8.48 (s, 1H), 7.81 (m, 1H), 7.64 (d, 1H), 7.57 (d, 1H), 1.68 (s, 3H).

### b) 4-(6-Bromo-pyridin-2-yl)-4-methyl-2,5-dioxo-imidazolidine-1,3-dicarboxylic acid di-tert-butyl ester

A solution of 5-(6-bromo-pyridin-2-yl)-5-methyl-imidazolidine-2,4-dione (22.8 g, 84.4 mmol), Boc₂O (58.8 ml, 55.3 g, 253.4 mmol) and DMAP (0.516 g, 4.22 mmol) in THF (600 ml) was stirred at rt for 4 h. The reaction mixture was concentrated to dryness, then taken up with EtOAc and filtered through silica. The silica cartridge was washed with EtOAc and THF, the combined filtrates were concentrated to leave the title compound as a pale yellow solid that was used in the next step without further purification.

HPLC Rt_{H11}=1.23 min; ESIMS: 470, 472 [(M+H)⁺]; ¹H NMR (400 MHz, CD₃OD): δ 7.82 (m, 1H), 7.65 (m, 2H), 2.11 (s, 3H), 1.60 (s, 9H), 1.30 (s, 9H).

### c) 2-Amino-2-(6-bromo-pyridin-2-yl)-propionic acid

A solution of 4-(6-bromo-pyridin-2-yl)-4-methyl-2,5-dioxo-imidazolidine-1,3-dicarboxylic acid di-tert-butyl ester (31.53 g, 67.0 mmol) in 2.5M aq. NaOH soln. (215 ml) was refluxed for 40 h. The reaction mixture was diluted with EtOAc (100 ml) and filtered. The filtrates were separated and the org. layer was washed with H₂O. The combined aq. layers were evaporated to dryness to leave a solid that was suspended in MeOH (350 ml) and stirred for 30 min. The suspension was filtered and the white precipitate was washed with MeOH. The filtrates were evaporated to leave a pale orange solid which was used for the next step without further purification.

HPLC Rt_{H11}=0.35-0.37 min; ESIMS: 245, 247 [(M+H)⁺]; ¹H NMR (400 MHz, CD₃OD): δ 7.60-7.51 (m, 2H), 7.36 (dd, 1H), 1.62 (s, 3H).

### d) 2-Amino-2-(6-bromo-pyridin-2-yl)-propan-1-ol

To a suspension of 2-amino-2-(6-bromo-pyridin-2-yl)-propionic acid (25.5 g, 72.8 mmol) and Boc₂O (33.8 ml, 31.8 g, 145.7 mmol) in acetonitrile (300 ml) and methanol (150 ml) was added tetramethylammonium hydroxide (65.1 ml of a 25% aq. soln., 182 mmol). The reaction was allowed to stir at rt for 6.5 h and was filtered. The filtrates were washed with MeOH and CH₃CN, then evaporated to leave an orange solid which was triturated with DCM and brine. The phases were separated and the aq. phase was 3× extracted with DCM. The combined org. phases were concentrated to leave crude 2-(6-bromo-pyridin-2-yl)-2-tert-butoxycarbonylamino-propionic acid as a pale brown foam (HPLC Rt_{Hii}=0.96-0.97 min, ESIMS: 345, 347 [(M+H)⁺]).

To a suspension of 2-(6-bromo-pyridin-2-yl)-2-tert-butoxycarbonylamino-propionic acid (14.1 g, 40.8 mmol) in THF (150 ml) was added portionwise NaBH₄ (3.45 g, 90.0 mmol) at 0° C. BF₃*Et₂O soln. (11.39 ml, 12.75 g, 90.0 mmol) was added dropwise over a period of 15 min and the reaction mixture was allowed to stir for 17 h at rt. In order to react remaining starting material, NaBH₄ (1.0 g, 26.43 mmol), and BF₃*Et₂O soln. (3.3 ml, 26.43 mmol) was added at 0° C. and the reaction mixture was stirred at rt for another 23 h. MeOH was added and the reaction mixture was stirred at 80° C. for 30 min, then cooled to rt and filtered. The filtrates were evaporated to leave a white foam which was taken up with EtOAc and 1N aq. NaOH soln. The phases were separated and the aq. phase was extracted three times with EtOAc. The combined org. phases were dried over Na₂SO₄, filtered and concentrated to leave [1-(6-bromo-pyridin-2-yl)-2-hydroxy-1-methyl-ethyl]-carbamic acid tert-butyl ester in a mixture with 2-amino-2-(6-bromo-pyridin-2-yl)-propan-1-ol. This mixture (7.5 g, 9.74 mmol) was rebocylated using Boc₂O (5.65 ml, 5.31 g, 24.34 mmol) and tetramethylammonium hydroxide (65.1 ml of a 25% aq. soln., 182 mmol) in acetonitrile (100 ml). After stirring for 1.5 h at rt, the reaction mixture was quenched with H₂O and diluted with EtOAc. The phases were separated and the aq. layer was twice reextracted with EtOAc. The combined org. layers were dried over Na₂SO₄, filtered and the solvent was removed to leave a yellow solid that was without further purification debocylated on a 8.1 g scale using 100 ml 4N aq. HCl. The reaction mixture was stirred at rt for 17 h, concentrated and the residue was taken up with H₂O and EtOAc. The phases were separated and the org. phase was washed with H₂O. The combined aq. phases were basified using 2N aq. NaOH soln. and then extracted with EtOAc. The phases were separated and the aq. phase was reextracted twice with EtOAc. The combined org. phases were dried over Na₂SO₄, filtered and concentrated to leave 2-amino-2-(6-bromo-pyridin-2-yl)-propan-1-ol as a colourless solid. HPLC Rt_{H11}=0.35 min; ESIMS: 231, 233 [(M+H)⁺]; ¹H NMR (400 MHz, DMSO-d₆): δ 7.73-7.63 (m, 2H), 7.45 (dd, 1H), 4.72-4.69 (m, 1H), 3.58 (dd, 1H), 3.40 (dd, 1H), 2.00 (br s, 2H), 1.26 (s, 3H).

### e) N-[1-(6-Bromo-pyridin-2-yl)-2-hydroxy-1-methyl-ethyl]-2-chloro-acetamide

To a solution of 2-amino-2-(6-bromo-pyridin-2-yl)-propan-1-ol (4.9 g, 21.2 mmol) in DCM (50 ml) was added K₂CO₃ (5.86 g, 42.4 mmol). The reaction mixture was cooled to 0° C. and 2-chloroacetyl chloride (2.55 ml, 3.59 g, 31.8 mmol) was added dropwise. The reaction mixture was allowed to warm to rt and to stir for 5 h. MeOH (20 ml) was added and stirring was continued at rt for 1 h. The reaction mixture was diluted with H₂O and DCM, the phases were separated and the aq. phase was twice extracted with DCM. The combined org. phases were dried over Na₂SO₄, filtered and the solvent was removed to leave N-[1-(6-bromo-pyridin-2-yl)-2-hydroxy-1-methyl-ethyl]-2-chloro-acetamide as an orange oil. HPLC Rt_{H4}=0.73-0.77 min; ESIMS: 307, 309 [(M+H)⁺]; ¹H NMR (400 MHz, DMSO-d₆): δ 8.35 (s, 1H), 7.70-7.66 (m, 1H), 7.48 (dd, 1H), 7.38 (dd, 1H), 5.05-5.02 (m, 1H), 4.14 (s, 2H), 3.68-3.66 (m, 2H), 1.55 (s, 3H).

### f) 5-(6-Bromo-pyridin-2-yl)-5-methyl-morpholin-3-one

To a solution of N-[1-(6-bromo-pyridin-2-yl)-2-hydroxy-1-methyl-ethyl]-2-chloro-acetamide in tert-butanol (90 ml) was added KOtBu and the reaction mixture was stirred at rt for 4 h. The reaction mixture was quenched with H₂O and diluted with EtOAc. The phases were separated and the aq. phase was twice extracted with EtOAc. The combined org. phases were washed with brine, dried over Na₂SO₄, filtered and the solvent was removed to leave the title compound as a pale yellow solid. HPLC Rt_{H11}=0.73 min; ESIMS: 271, 273 [(M+H)⁺]; ¹H NMR (400 MHz, DMSO-d₆): δ 8.72 (s, 1H), 7.82-7.78 (m, 1H), 7.57-7.51 (m, 2H), 4.10 (d, 2H), 4.00 (d, 1H), 3.65 (d, 1H), 1.42 (s, 3H).

### g) 5-(6-Bromo-pyridin-2-yl)-5-methyl-morpholine-3-thione

A mixture of 5-(6-bromo-pyridin-2-yl)-5-methyl-morpholin-3-one (4.65 g, 17.15 mmol) and P₂S₆ (4.57 g, 20.58 mmol) in pyridine (60 ml) was stirred at 80° C. under N₂ for 6 h. The reaction mixture was cooled to rt and diluted with 0.5N aq. HCl and EtOAc. The phases were separated and the aq. phase was twice extracted with EtOAc. The combined org. phases were washed with brine, dried over Na₂SO₄, filtered and concentrated. The title compound was obtained as a pale yellow solid after flash chromatography on silica gel (cyclohexane/EtOAc 100:0 to 75:25). HPLC Rt_{Hii}=0.89 min; ESIMS: 287, 289 [(M+H)⁺]; ¹H NMR (400 MHz, DMSO-d₆): δ 11.15 (s, 1H), 7.86-7.82 (m, 1H), 7.61 (dd, 1H), 7.39 (dd, 1H), 4.44-4.34 (d, 2H), 4.13 (d, 1H), 3.74 (d, 1H), 1.52 (s, 3H).

### h) 5-(6-Bromo-pyridin-2-yl)-5-methyl-5,6-dihydro-2H-[1,4]oxazin-3-ylamine

A mixture of 5-(6-bromo-pyridin-2-yl)-5-methyl-morpholine-3-thione (1.4 g, 4.88 mmol) in 7N NH₃/MeOH (20.89 ml, 146 mmol) was stirred at 50° C. for 3 d in an autoclave. The reaction mixture was evaporated to dryness and purified by FC (gradient cyclohexane:EtOAc 75:25 to 50:50, then +10% Et₃N, finally MeOH+10% Et₃N) to obtain the crude title compound that was further purified by washing with DCM. HPLC Rt_{H11}=0.54 min; ESIMS: 270, 272 [(M+H)⁺]; ¹H NMR (400 MHz, DMSO-d₆): δ 8.51 (br s, 2H), 7.83-7.79 (m, 1H), 7.63-7.61 (m, 2H), 4.45 (s, 2H), 4.11 (d, 1H), 3.84 (d, 1H), 1.51 (s, 3H).

### i) [5-(6-Bromo-pyridin-2-yl)-5-methyl-5,6-dihydro-2H-[1,4]oxazin-3-yl]-carbamic acid tert-butyl ester

A suspension of 5-(6-bromo-pyridin-2-yl)-5-methyl-5,6-dihydro-2H-[1,4]oxazin-3-ylamine (1.100 g, 4.07 mmol), Boc₂O (1.229 ml, 1.155 g, 5.29 mmol) and DIPEA (1.067 ml, 0.789 g, 6.11 mmol) in DCM (30 ml) was stirred at rt for 20 h. The reaction mixture was diluted with H₂O and DCM. The phases were separated and the aq. phase was twice reextracted with DCM. The combined org. phases were washed with brine, dried over Na₂SO₄, filtered and concentrated to yield the title compound as a colourless solid that was used in the next step without further purification. HPLC Rt_{H11}=0.92 min; ESIMS: 370, 372 [(M+H)⁺].

### j) (+)- and (−)-5-(6-Amino-pyridin-2-yl)-5-methyl-5,6-dihydro-2H-[1,4]oxazin-3-yl]-carbamic acid tert-butyl ester

A mixture of [5-(6-Bromo-pyridin-2-yl)-5-methyl-5,6-dihydro-2H-[1,4]oxazin-3-yl]-carbamic acid tert-butyl ester (986 mg, 2.66 mmol), cyclohexanedimethyldiamine (0.420 ml, 379 mg, 2.66 mmol), sodium ascorbate (211 mg, 1.07 mmol), NaN₃ (1385 mg, 21.31 mmol) and CuI (203 mg, 1.07 mmol) in ethanol/water (22.0/8.8 ml) was degassed with N₂ in a dry ice/EtOH bath. The reaction mixture was then stirred at 45° C. for 4 h. The reaction mixture was allowed to warm to rt and filtered through hyflo, rinsed with EtOAc and concentrated. Flash chromatography on silica gel (cyclohexane/EtOAc gradient 0-3 min 100:0, 3-25 min 60:40, 40-52 min 50:50) yielded the title compound. HPLC Rt_{H11}=0.60, 0.66 min; ESIMS: 305 [(M−H)⁺]; ¹H NMR (400 MHz, DMSO-d₆): δ 7.77-7.73 (m, 1H), 6.81-6.76 (m, 2H), 4.71-4.63 (m, 1H), 4.70-4.56 (m, 2H), 4.06-3.96 (m, 2H), 1.69 (s, 3H), 1.51 (s, 9H).

Racemic 5-(6-amino-pyridin-2-yl)-5-methyl-5,6-dihydro-2H-[1,4]oxazin-3-yl]-carbamic acid tert-butyl ester was separated into the pure enantiomers by preparative chiral HPLC (column: Chiralpak AS; solvent: n-heptane/ethanol/isopropylamine=80:12:8; flow: 70 ml/min; detection at 220 nm). Enantiomer 1: [α]_{D}=−138.5° (c=1.00, MeOH). Enantiomer 2: [α]_{D}=+ 141.5° (c=1.03, MeOH). (−)-Enantiomer 1 was used for the following steps, its configuration was assigned (R) in analogy to similar structures of which the configuration has been determined by x-ray crystallography.

### k) ((R)-5-{6-[(5-Bromo-pyridine-2-carbonyl)-amino]-pyridin-2-yl}-5-methyl-5,6-dihydro-2H-[1,4]oxazin-3-yl)-carbamic acid tert-butyl ester

To a solution of 5-bromopyridine-2-carboxylic acid (34.5 mg, 0.171 mmol) in DCM (2 ml) was added 1-chloro-N,N,2-trimethylpropenylamine (0.045 ml, 45.7 mg, 0.342 mmol) and the reaction mixture was stirred at 0° C. for 1 h. The reaction mixture was then added dropwise to a dry solution of (−)-5-(6-amino-pyridin-2-yl)-5-methyl-5,6-dihydro-2H-[1,4]oxazin-3-yl]-carbamic acid tert-butyl ester (enantiomer 1 from procedure step j) above, 47.6 mg, 0.155 mmol) and NEt₃ (0.048 ml, 34.6 mg, 0.342 mmol) in DCM (2 ml) at 0° C. The reaction mixture was allowed to warm to rt and to stir for 20 min at rt. The reaction mixture was diluted with DCM and quenched with H₂O. The phases were separated and the aq. phase was extracted with DCM. The combined org. phases were washed with brine, dried over Na₂SO₄, filtered and twice purified by HPLC (Alltech Grom Saphir65 Si 10 μM column 150×30 mm, gradient 1 n-heptane:EtOAc 0-1.2 min 85:15, 1.2-9 min 0:100, 9-12 min 0:100, gradient 2 n-heptane:EtOAc:MeOH 0-1.2 min 47:50:3, 1.2-9 min 0:60:40, 9-12 min 0:60:40, flow 50 ml/min, detection 254 nm]. HPLC Rt_{H11}=1.10 min; ESIMS: 490, 492 [(M+H)⁺].

### l) 5-Bromo-pyridine-2-carboxylic acid [64(R)-5-amino-3-methyl-3,6-dihydro-2H-[1,4]-oxazin-3-yl)-pyridin-2-yl]-amide

To a solution of 5-{6-[(5-bromo-pyridine-2-carbonyl)-amino]-pyridin-2-yl}-5-methyl-5,6-dihydro-2H-[1,4]oxazin-3-yl)-carbamic acid tert-butyl ester (43 mg, 0.088 mmol) in DCM (270 μl) was added TFA (270 μl, 400 mg, 3.51 mmol) and the reaction mixture was stirred at rt for 1 h. The reaction mixture was quenched with 1M aq. NaHCO₃ soln. and diluted with DCM. The phases were separated and the aq. phase was twice reextracted with DCM. The combined org. phases were dried over Na₂SO₄, filtered, concentrated and purified by manual flash chromatography (NH₃-desactivated silica gel, hexane:DCM:MeOH 10:10:1 then DCM:MeOH 10:1, then +0.1% NH₃ and finally MeOH+1% NH₃) to yield the title compound as a colourless solid. To a solution of the free base in DCM was added 1 eq. 2N HCl/Et₂O and the resulting hydrochloride salt was collected. HPLC Rt_{H11}=0.76 min; ESIMS: 390, 392 [(M+H)⁺]; ¹H NMR (400 MHz, CD₃OD): δ 8.88 (d, 1H), 8.38 (d, 1H), 8.33 (dd, 1H), 8.27-8.24 (m, 1H), 8.24-7.98 (m, 1H), 7.33 (d, 1H), 4.69 (d, 1H), 4.67 (d, 1H), 4.30 (d, 1H), 4.11 (d, 1H), 1.78 (s, 3H).

### Example 258

### 5-Bromo-pyridine-2-carboxylic acid [6-(5-amino-3-methyl-3,6-dihydro-2H-[1,4]-oxazin-3-yl)-pyridin-2-yl]-amide

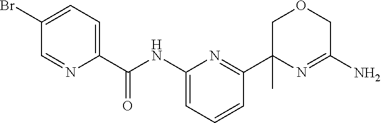 [SMILES: CC1(c2cccc(NC(=O)c3ccc(Br)cn3)n2)COCC(N)=N1]

The racemate of Example 257 can be prepared by a procedure analogous to that used in Example 257 completing the synthesis using the racemic mixture obtained in step j) of Example 257 and has the same analytical data.

### Example 259

### 5-{6-[(5-Chloro-pyridine-2-carbonyl)-amino]-pyridin-2-yl}-5-fluoromethyl-5,6-dihydro-2H-[1,4]oxazin-3-yl-ammonium trifluoro acetate

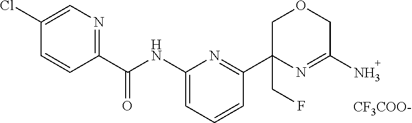

### a) 2-(6-Bromo-pyridin-2-yl)-malonic acid diethyl ester

Lithium diisopropylamide (2.0 M solution in heptane/THF/ethyl benzene, 581.3 ml) was taken in dry THF (400 mL) and cooled to −78° C. 2-Bromo-6-methylpyridine (50.0 g, 296.64 mmol) was added slowly to the LDA solution at the same temperature for 15 min., allowed to stir constantly for 30 min. Ethylchloroformate (94.62 g, 871.94 mmol) in dry THF (50 ml) was then added to the stirred contents drop wise and allowed the reaction mass to stir at −78° C. for 2 h. Reaction mixture was quenched with saturated ammonium chloride solution and product formed was extracted with ethyl acetate by washing with water, brine and dried over anhy. Na₂SO₄. Organic layer was concentrated under reduced pressure and the crude product was purified by column chromatography using 10% ethyl acetate in hexane to furnish title compound as a brown colored liquid. Yield=65.0 g (71.4%). TLC (10% ethyl acetate in hexane: Rf=0.31). LCMS: Rt_{Hg}=1.866; [M+1]=315.8 and 317.9; HPLC: Rt_{H15}=4.636 min (48%); ¹H NMR (400 MHz, CDCl₃): δ 7.587 (t, 1H), 7.47 (d, 1H), 7.27 (d, 1H), 3.91 (s, 1H), 4.25-4.09 (m, 4H), 1.24 (t, 6H).

### b) (6-Bromo-pyridin-2-yl)-acetic acid

2-(6-Bromo-pyridin-2-yl)-malonic acid diethyl ester (64.0 g, 202.4 mmol) was added to a solution of potassium carbonate (279.8 g, 2024 mmol) in water (400 ml) at rt and the reaction mixture was heated to reflux at 100° C. for 36 h. The reaction mixture was treated with sat. ammonium chloride solution and the product formed was extracted with ethyl acetate (3×800 ml), washed with brine (10 ml). Organic layer was concentrated under reduced pressure to obtain title compound as a pale brown solid. Yield=34.0 g (77.7%). TLC (50% ethyl acetate in hexane: Rf=0.11). LCMS: Rt_{H8}=0.45; [M+1]=216.0 and 218.0.

### c) (6-Bromo-pyridin-2-yl)-acetic acid ethyl ester

To a solution of (6-bromo-pyridin-2-yl)-acetic acid (34.0 g, 158.14 mmol) in ethanol (300 ml) was added conc. H₂SO₄ (5.0 ml) and heated to reflux for 12 h. The reaction mixture was cooled to rt and concentrated under reduced pressure to dryness. Water was added to the residue and the product was extracted with ethyl acetate. Organic layer was washed with brine, dried over anhy. Na₂SO₄ and concentrated under reduced pressure to furnish the crude product. Column chromatography purification furnished the title compound as a brown liquid. Yield=31.2 g (82%). TLC (20% ethyl acetate in hexane: Rf=0.51). LCMS: Rt_{H8}=0.996, [M+1]⁺=244.0 and 246.0; HPLC: Rt_{H15}=3.87 min (97.2%); ¹H NMR (400 MHz, CDCl₃): δ 7.53 (t, 1H), 7.39 (d, 1H), 7.28 (d, 1H), 4.19 (q, 2H), 3.83 (s, 2H), 1.25 (t, 3H).

### d) 2-(6-Bromo-pyridin-2-yl)-3-hydroxy-2-hydroxymethyl-propionic acid ethyl ester

To the solution of para formaldehyde (9.6 g, 319.55 mmol) and sodium ethoxide (0.87 g, 12.784 mmol) in dry THF (250 ml) was added (6-bromo-pyridin-2-yl)-acetic acid ethyl ester (31.2 g, 127.82 mmol) at 0° C. to −10° C. and allowed the reaction mixture to stir at same temperature for 4 h. Solids formed in the reaction mixture were filtered and washed with ethyl acetate and the filtrate was concentrated to obtain crude product as a brown liquid. Yield=30.0 g (crude). TLC (30% ethyl acetate in hexane: Rf=0.28).

LCMS: Rt_{H8}=0.702, M+1=304.0 and 306.0.

### e) 2-(6-Bromo-pyridin-2-yl)-3-methoxymethoxy-2-methoxymethoxymethyl-propionic acid ethyl ester

To the solution of 2-(6-bromo-pyridin-2-yl)-3-hydroxy-2-hydroxymethyl-propionic acid ethyl ester (30.0 g, 98.638 mmol) in dry THF (250 ml) was added tetrabutyl ammonium bromide (15.8 g, 49.319 mmol) and di-isopropyl ethyl amine (127.47 g, 163.0 ml) followed by methoxymethyl chloride was added drop wise at RT. Resultant reaction contents were refluxed at 65° C. for 3 h and cooled to RT. Reaction mixture was concentrated under reduced pressure and purified by column chromatography over silica gel using 10% ethyl acetate in hexane to furnish title compound as a brown liquid. Yield=20.4 g (52%). TLC (30% ethyl acetate in hexane: Rf=0.55). LCMS: Rt_{H8}=1.639, [M+1]⁺=392.0 and 394.0; ¹H NMR (400 MHz, CDCl₃): δ 7.49 (t, 1H), 7.35 (d, 1H), 7.22 (d, 1H), 4.62-4.47 (m, 4H), 4.24-4.16 (m, 6H), 3.25 (s, 6H), 1.23 (t, 3H).

### f) 2-(6-Bromo-pyridin-2-yl)-3-methoxymethoxy-2-methoxymethoxymethyl-propionic acid

Lithium hydroxide (10.69 g, 254.95 mmol) was added to a solution of 2-(6-bromo-pyridin-2-yl)-3-methoxymethoxy-2-methoxymethoxymethyl-propionic acid ethyl ester (20.0 g, 50.99 mmol) in ethanol (100 ml) and water (100 ml) at rt and the reaction mixture was allowed to stir overnight. The reaction mass was concentrated under reduced pressure and acidified with dilute HCl and at 0° C. The product was extracted with ethyl acetate, washed with minimum amount of brine. Organic layer was concentrated under reduced pressure to obtain title compound as a brown liquid. Yield=18.0 g. TLC (50% ethyl acetate in hexane: Rf=0.05). LCMS: Rt_{H8}=1.383, [M+1]=364.0 and 366.0; HPLC: Rt_{H15}=3.844 min (49%) and 3.885 min. (22%).

### g) 1-(6-Bromo-pyridin-2-yl)-2-methoxymethoxy-1-methoxymethoxymethyl-ethylamine

To a suspension of 2-(6-bromo-pyridin-2-yl)-3-methoxymethoxy-2-methoxymethoxymethyl-propionic acid (18.0 g) in toluene (150 ml) diphenyl phosphoryl azide (4.08 g, 148.27 mmol) and triethylamine (14.97 g [20.6 ml], 148.27 mmol) were added at rt and stirred at 100° C. for 15 h. Reaction mixture was cooled to rt and concentrated under reduced pressure. The residue obtained was dissolved in THF (600 ml) and 20% NaOH solution was added at rt and stirred for 1 h. Solvent was removed under reduced pressure and the product formed was extracted with ethyl acetate. Organic layer was washed with brine, followed by dried over MgSO₄. The organic portion was concentrated under reduced pressure and column chromatographic purification of the crude product using 35% ethyl acetate in hexane furnished title compound as a brown liquid. Yield=15.0 g (88% [2 steps]). TLC (70% ethyl acetate in hexane: Rf=0.51). LCMS: Rt_{H8}=0.28, [M+1]=335.0 and 337.0.

### h) N-[1-(6-Bromo-pyridin-2-yl)-2-methoxymethoxy-1-methoxymethoxymethyl-ethyl]-2-chloro-acetamide

To a solution of 1-(6-bromo-pyridin-2-yl)-2-methoxymethoxy-1-methoxymethoxymethyl-ethylamine (15.0 g, 44.75 mmol) in DCM (150 ml) was added aqueous Na₂CO₃ solution (10.91 g, 102.925 mmol in water, 30 ml) was added at 0° C. and stirred for 10 min. Chloroacetylchloride (5.56 g, 49.225 mmol) was added to the resultant reaction mixture at 0° C. stirring continued for 1 h at ambient temperature. Reaction mixture was diluted with DCM (˜1 l), and worked up the reaction mixture by washing with water, brine and dried over anhy. Na₂SO₄. Organic layer was separated and concentrated under reduced pressure to obtain title compound as a brown liquid. Yield=9.0 g (48%). TLC (50% ethyl acetate in hexane: Rf=0.54). LCMS: Rt_{H8}=1.341, [M+1]=411.0 and 413.0; HPLC: Rt_{H15}=4.27 min (50.4%); ¹H NMR (400 MHz, CDCl₃): δ 8.11 (d, 1H), 7.57-7.52 (m, 1H), 7.46-7.37 (m, 2H), 4.59-4.52 (m, 4H), 4.23-4.17 (m, 4H), 4.09-4.04 (m, 2H), 3.21 (s, 6H).

### i) N-[1-(6-Bromo-pyridin-2-yl)-2-hydroxy-1-hydroxymethyl-ethyl]-2-chloro-acetamide

To a solution of N-[1-(6-bromo-pyridin-2-yl)-2-methoxymethoxy-1-methoxymethoxymethyl-ethyl]-2-chloro-acetamide (9.0 g, 21.861 mmol) in ethanethiol (30 ml) and BF₃.Et₂O (9.3 g, 141.93 mmol) was added at 0° C. and stirred for 10 min. Stirring was continued for 3 h at rt. Reaction mixture was quenched with saturated NaHCO₃ solution and the product formed was extracted with ethyl acetate. Organic layer was separated and washed with brine solution, followed by drying over anhydrous Na₂SO₄. Organic layer was concentrated under reduced pressure and purified by column chromatography using 2% methanol in chloroform to obtain title compound as a brown liquid. Yield=5.5 g (77%). TLC (10% methanol in chloroform: Rf=0.51). LCMS: Rt_{H8}=0.916, [M+1]=322.9 and 324.8; HPLC Rt_{H15}=5.931 min (89%); ¹H NMR (400 MHz, CDCl₃): δ 8.27 (s, 1H), 7.56 (t, 1H), 7.42-7.39 (m, 2H), 4.38 (s, 2H), 4.09-4.07 (m, 4H), 3.95 (d, 2H).

### j) 5-(6-Bromo-pyridin-2-yl)-5-hydroxymethyl-morpholin-3-one

To the solution of N-[1-(6-bromo-pyridin-2-yl)-2-hydroxy-1-hydroxymethyl-ethyl]-2-chloro-acetamide (5.4 g, 16.69 mmol) in t-BuOH (80 ml) was added t-BuOK (2.06 g, 18.38 mmol) at rt followed by sodium iodide (0.25 g, 1.669 mmol) and allowed the reaction mixture to stir at 90° C. for 1 h. The reaction mixture was concentrated under reduced pressure and the residue obtained was treated with water. Compound present in the residue was extracted with ethyl acetate (2×100 ml). Organic portion was washed with brine, dried over Na₂SO₄ and concentrated under reduced pressure to obtain gummy compound. Further trituration of the formed product with n-pentane (5.0 ml) and diethyl ether (5.0 ml) furnished title compound as a pale yellow gummy compound. Yield=3.3 g (68.8%). TLC (50% ethyl acetate in hexane Rf=0.21). LCMS: Rt_{H8}=0.155, [M+1]=286.9 and 288.8; HPLC Rt_{H15}=3.03 min (69.8%); ¹H NMR (400 MHz, CDCl₃): δ 8.40 (s, 1H), 7.79 (t, 1H), 7.57 (d, 2H), 5.11 (s, 1H), 4.15 (d, 1H), 3.99 (d, 2H), 3.88 (d, 1H), 3.71-3.60 (m, 2H).

### k) 5-(6-Bromo-pyridin-2-yl)-5-fluoromethyl-morpholin-3-one

To a solution of 5-(6-bromo-pyridin-2-yl)-5-hydroxymethyl-morpholin-3-one (2.8 g, 9.756 mmol) in dry THF (30 ml), diethylamino sulfur trifluoride (4.72 g, 29.268 mmol) was added at rt and stirring continued for 4 h. Na₂CO₃ was then added to the resultant reaction mixture and stirred for further 30 min. The reaction mixture was concentrated under reduced pressure and the product was extracted with ethyl acetate. Organic layer was washed with brine, dried over anhy. Na₂SO₄ and concentrated under reduced pressure to obtain crude compound as gummy residue. Purification of the crude product with 45% ethyl acetate in hexane solvent system furnished the title compound as off-white solid. Yield=1.15 g (40%). TLC (70% ethyl acetate in hexane: Rf=0.49). LCMS: Rt_{H8}=0.383, [M+1]⁺=289 and 289; HPLC Rt_{H15}=3.27 min (84%); ¹H NMR (400 MHz, CDCl₃): δ 7.62 (t, 1H), 7.49 (dd, 1H), 7.32 (d, 1H), 7.09 (br. s, 1H), 4.92 (dd, 1H), 4.52 (dd, 1H), 4.32-4.17 (m, 3H), 3.98-3.93 (dd, 1H); ¹⁹F NMR (376.2 MHz): δ −255.65 (t, 1F).

### l) 5-Chloro-pyridine-2-carboxylic acid [6-(3-fluoromethyl-5-oxo-morpholin-3-yl)-pyridin-2-yl]-amide

A mixture of 4,5-Bis(diphenyl phosphino)-9,9-dimethyl xanthene (0.04 g, 0.069 mmol), tris(dibenzylidene-acetone) di palladium(0) (0.032 g, 0.035 mmol) and cesium carbonate (0.678 g, 2.083 mmol) were taken in 1,4-dioxane and degassed with argon for 10 min. 5-(6-Bromo-pyridin-2-yl)-5-fluoromethyl-morpholin-3-one (0.2 g, 0.694 mmol) followed by 5-chloropicolinamide (0.119 g, 0.764 mmol) were added to the resultant reaction mixture and degassed with argon for further 5 min. Reaction mixture was then heated at 80° C. for 16 h and cooled to rt. Reaction contents were treated with water and product was extracted with ethyl acetate, washed with brine and dried over anhy. Na₂SO₄. The organic layer was concentrated under reduced pressure to obtain liquid which was triturated with n-pentane to furnish title compound as a off-white solid. Yield=0.24 g (crude). TLC (50% ethyl acetate in hexane: Rf=0.45). LCMS: Rt_{H6}=1.215, [M+1]=365.1 and 366.9; HPLC Rt_{H15}=4.367 min (53%).

### m) 5-Chloro-pyridine-2-carboxylic acid [6-(3-fluoromethyl-5-thioxo-morpholin-3-yl)-pyridin-2-yl]-amide

To a solution of 5-chloro-pyridine-2-carboxylic acid [6-(3-fluoromethyl-5-oxo-morpholin-3-yl)-pyridin-2-yl]-amide (0.24 g, 0.658 mmol) in THF (10.0 ml), Lawesson's reagent (0.798 g, 1.974 mmol) was added at rt and heated at reflux temperature for 24 h. Reaction mass was concentrated under reduced pressure. The crude compound was directly purified by column chromatography using 23% ethyl acetate in hexane to furnish title compound as off-white solid. Yield=0.19 g (72% [2 steps]). TLC (50% ethyl acetate in hexane: Rf=0.71). LCMS: Rt_{H6}=1.578, [M+1]⁺=381.1 and 382.9.

### n) 5-{6-[(5-Chloro-pyridine-2-carbonyl)-amino]-pyridin-2-yl}-5-fluoromethyl-5,6-dihydro-2H-[1,4]oxazin-3-yl-ammonium trifluoro acetate

To a solution of 5-chloro-pyridine-2-carboxylic acid [6-(3-fluoromethyl-5-thioxo-morpholin-3-yl)-pyridin-2-yl]-amide (0.19 g, 0.499 mmol) in methanol (2.0 ml), 10% ammonia in methanol (8.0 ml) was added at 0° C. in a sealed tube and stirred at rt for 24 h. Reaction mass was concentrated under reduced pressure and directly purified by preparative HPLC. Conditions: column: C18-ZORBAX 21.2×150 mm; 5 μm. mobile phase: 0.1% TFA in water (A)/ACN; flow: 20 ml/min. Yield: 86 mg (36%). M.P: 216-218° C. TLC (20% methanol in chloroform: Rf=0.45). LCMS: Rt_{H6}=0.194 [M+1]=364.0 and 366.1; HPLC Rt_{H15}=3.222 min (98.7%); ¹H NMR (400 MHz, DMSO-d₆): δ 10.85 (s, 1H), 10.37 (s, 1H), 9.37 (s, 1H), 8.50-8.79 (m, 2H), 8.31-8.23 (m, 3H), 8.06 (t, 1H), 7.37 (d, 1H), 4.96 (dd, 1H), 4.85 (dd, 1H), 4.62 (dd, 2H), 4.25-4.16 (m, 2H). Product formation was also confirmed by 2D NMR-ROESY.

### Examples 260 to 263

The compounds listed in Table 27 were prepared by a procedure analogous to those used in Example 259.

TABLE 27

| | | | MS |
|---|---|---|---|
| | | ¹H-NMR | [m/z; |
| Example | Compound | (δ; DMSO-d₆) | (M + 1)⁺] |
| 260 | 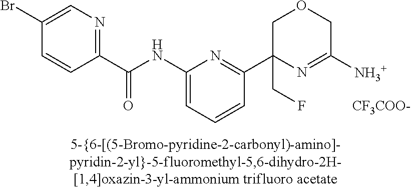 | 10.74 (s, 1H), 10.34 (s, 1H), 9.35 (s, 1H), 8.92 (t, 1H), 8.69 (s, 1H), 8.39 (dd, 1H), 8.29 (dd, 1H), 8.17 (dd, 1H), 8.05 (t, 1H), 7.36 (d, 1H), 4.96 (dd, 1H), 4.86 (dd, 1H), 4.62 (dd, 2H), 4.25- 4.15 (m, 2H). | LCMS: Rt_{H9} = 0.239 [M + 1] = 408.0, 410.0 |
| 261 | 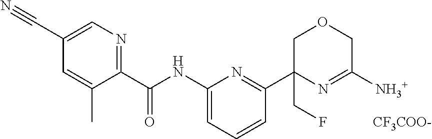 | 9.01 (s, 1H), 8.43 (s, 1H), 8.11 (d, 1H), 7.874 (t, 1H), 7.40 (d, 1H), 4.59-4.45 (m, 2H), 4.03-3.85 (m, 6H), 2.61 (s, 3H), 1.89 (s, 3H). | LCMS: Rt_{H9} = 0.127; [M + 1] = 369.4 |
| | 5-{6-[(5-Cyano-3-methyl-pyridine-2- | | |
| | carbonyl)-amino]-pyridin-2-yl}-5- | | |
| | fluoromethyl-5,6-dihydro-2H-[1,4]oxazin-3- | | |
| | yl-ammonium trifluoro acetate | | |
| 262 | 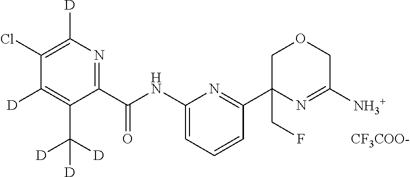 | 10.80 (s, 1H), 10.54 (s, 1H), 9.35 (s, 1H), 8.76 (s, 1H), 8.25 (d, 1H), 8.02 (t, 1H), 7.33 (d, 1H), 4.99-4.76 (m, 2H), 4.61 (m, 2H), 4.18 (t, 2H). | LCMS: Rt_{H9} = 0.208; [M + 1] = 383.3 |
| | 5-{6-[(4,6-Dideutero-5-chloro-3- | | |
| | trideuteromethyl-pyridine-2-carbonyl)- | | |
| | amino]-pyridin-2-yl}-5-fluoromethyl-5,6- | | |
| | dihydro-2H-[1,4]oxazin-3-yl-ammonium | | |
| | trifluoro acetate | | |
| 263 | 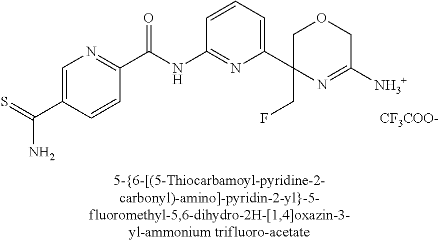 | 10.82 (s, 1H), 10.46 (s, 1H), 10.33 (s, 1H), 9.96 (s, 1H), 9.36 (s, 1H), 90.1-9.09 (m, 1H), 8.73 (s, 1H), 8.44 (dd, 1H), 8.28 (dd, 2H), 8.06 (t, 1H), 7.37 (d, 1H), 5.03- 4.78 (m, 2H), 4.61 (dd, 2H), 4.26-4.16 (m, 2H). | LCMS: Rt_{H9} = 0.153; [M + 1] = 389.1 |

### Example 264

### 5-Cyano-3-methyl-pyridine-2-carboxylic acid [64(3R,6R)-5-amino-3,6-dimethyl-6-trifluoro-methyl-3,6-dihydro-2H-[1,4]oxazin-3-yl)-pyridin-2-yl]-amide

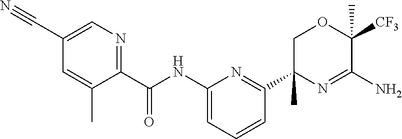 [SMILES: Cc1cc(C#N)cnc1C(=O)Nc1cccc([C@]2(C)CO[C@@](C)(C(F)(F)F)C(N)=N2)n1]

### a) 4-(6-Bromo-pyridin-2-yl)-4-methyl-2-oxo-2lambda*4*-[1,2,3]oxathiazolidine-3-carboxylic acid tert-butyl ester

To an at 0° C. precooled solution of thionyl chloride (3.42 ml, 5.57 g, 46.8 mmol) in pyridine (9.46 ml, 9.25 g, 117.0 mmol) was added dropwise a solution of [1-(6-bromo-pyridin-2-yl)-2-hydroxy-1-methyl-ethyl]-carbamic acid tert-butyl ester (see Example 257 step d), 7.75 g, 23.4 mmol) in DCM (230 ml). The reaction mixture was allowed to stir for 1 h at rt, then 0.5 N aq. HCl and DCM were added, the phases were separated and the aq. phase was twice reextracted with DCM. The combined org. phases were washed with brine, dried over Na₂SO₄, filtered and concentrated to leave the title compound (mixture of diastereomers) as an orange solid. HPLC Rt_{H11}=1.16, 1.20 min (diastereomers); ESIMS: 377, 379 [(M+H)⁺].

### b) 4-(6-Bromo-pyridin-2-yl)-4-methyl-2,2-dioxo-2lambda*6*-[1,2,3]oxathiazolidine-3-carboxylic acid tert-butyl ester

To a solution of 4-(6-bromo-pyridin-2-yl)-4-methyl-2-oxo-2lambda*4*-[1,2,3]oxathiazolidine-3-carboxylic acid tert-butyl ester (8.83 g, 23.4 mmol) in acetonitrile (60 ml) and H₂O (30.0 ml) was added RuCl₃ hydrate (0.971 g, 4.68 mmol) and NaIO₄ (10.01 g, 46.8 mmol). The reaction mixture was stirred at 0° C. for 2 h. H₂O and DCM were added, the phases were separated and the aq. phase was twice reextracted with DCM. The combined org. phases were washed with brine, dried over Na₂SO₄, filtered and concentrated. The residue was dissolved in DCM and filtered through silica gel, the filtrate was evaporated and the residue was triturated with TBDME (10 ml) and n-hexane (100 ml). The resulting precipitate was filtered and washed with n-hexane to yield the title compound as a colourless crystalline solid. HPLC Rt_{H11}=1.16 min; ESIMS: 393, 395 [(M+H)⁺]; ¹H NMR (400 MHz, CDCl₃): δ 7.63-7.59 (m, 1H), 7.47-7.42 (m, 2H), 4.73 (d, 1H), 4.47 (d, 1H), 2.00 (s, 3H), 1.52 (s, 9H).

### c) (R)-2-[(RS)-2-(6-Bromo-pyridin-2-yl)-2-tert-butoxycarbonylamino-propoxy]-3,3,3-trifluoro-2-methyl-propionic acid ethyl ester

At 0° C., NaH (0.508 g of a 60% dispersion in mineral oil, 12.69 mmol) was added to a solution of 4-(6-bromo-pyridin-2-yl)-4-methyl-2,2-dioxo-2lambda*6*-[1,2,3]oxathiazolidine-3-carboxylic acid tert-butyl ester (3.84 g, 9.76 mmol) and (R)-3,3,3-trifluoro-2-hydroxy-2-methyl-propionic acid ethyl ester (2.54 g, 13.67 mmol) in DMF (10 ml, soln. predried over 4A mol. sieves). The reaction mixture was allowed to stir at rt for 30 min, then at 60° C. for 17 h. The reaction mixture was quenched with H₂O and diluted with 1N aq. HCl and EtOAc. The phases were separated and the aq. phase was twice reextracted with EtOAc. The combined org. phases were washed with brines, dried over Na₂SO₄, filtered and concentrated. Flash chromatography on silica gel (cyclohexane:EtOAc, gradient 0-5 min 100:0, 5-30 min 90:10, 30-40 min 90:10, 40-50 min 80:20, 50-55 min 80:20) yielded the title compound (diastereomer mixture) as a clear oil. HPLC Rt_{H11}=1.39 min; ESIMS: 499, 501 [(M+H)⁺].

### d) [(RS)-1-(6-Bromo-pyridin-2-yl)-2-((R)-1-carbamoyl-2,2,2-trifluoro-1-methyl-ethoxy)-1-methyl-ethyl]-carbamic acid tert-butyl ester

A solution of (R)-2-[(RS)-2-(6-bromo-pyridin-2-yl)-2-tert-butoxycarbonylamino-propoxy]-3,3,3-tri-fluoro-2-methyl-propionic acid ethyl ester (3.0 g, 6.01 mmol) in 7N NH₃/MeOH (6.5 ml) was stirred in a sealed glass vial at 55° C. for 72 h. The reaction mixture was concentrated to leave the title compound as a colourless solid that was used in the next step without further purification. HPLC Rt_{H11}=1.12, 1.14 min (diastereomers); ESIMS: 470, 472 [(M+H)⁺].

### e) [(RS)-1-(6-Bromo-pyridin-2-yl)-2-((R)-1-cyano-2,2,2-trifluoro-1-methyl-ethoxy)-1-methyl-ethyl]-carbamic acid tert-butyl ester

To an at 0° C. percooled solution of [(RS)-1-(6-bromo-pyridin-2-yl)-2-((R)-1-carbamoyl-2,2,2-trifluoro-1-methyl-ethoxy)-1-methyl-ethyl]-carbamic acid tert-butyl ester (2.18 g, 4.64 mmol) and NEt₃ (1.615 ml, 1.173 g, 11.59 mmol) in DCM (30 ml) was added dropwise TFAA (0.773 ml, 1.168 g, 5.56 mmol). After stirring for 5 min at 0° C., then for 1 h at rt the reaction mixture was diluted with sat. aq. Na₂CO₃ soln. and with DCM. The phases were separated and the aq. phase was twice reextracted with DCM. The combined org. phases were dried over Na₂SO₄, filtered and concentrated to leave a pale yellow oil which was stirred with 7N NH₃/MeOH for 5 min. The mixture was evaporated to dryness and purified by flash chromatography (cyclohexane:EtOAc 0-3 min 100:0, 3-35 min 65:35) to yield the title compound as a clear oil.

HPLC Rt_{H11}=1.30 min; ESIMS: 452, 454 [(M+H)⁺]; ¹H NMR (400 MHz, CDCl₃): δ 7.59-7.53 (m, 1H), 7.42-7.36 (m, 2H), 5.66 (br s, 1H), 4.41-4.31 (m, 1H), 4.25-4.18 (m, 1H), 1.71 (d, 3H), 1.66 (d, 3H), 1.43 (s, 9H).

### f) (R)-2-[(RS)-2-Amino-2-(6-bromo-pyridin-2-yl)-propoxy]-3,3,3-trifluoro-2-methyl-propionitrile

A solution of [(RS)-1-(6-bromo-pyridin-2-yl)-2-((R)-1-cyano-2,2,2-trifluoro-1-methyl-ethoxy)-1-methyl-ethyl]-carbamic acid tert-butyl ester (0.456 g, 1.008 mmol) and TFA (1.554 ml, 2.299 g, 20.17 mmol) in DCM (5 ml) was stirred at rt for 30 min, concentrated and triturated with 7N NH₃/MeOH at rt for 20 min and again concentrated to give the title compound that was used for the next step without further purification. HPLC Rt_{H11}=0.69, 0.73 min (diastereomers); ESIMS: 352, 354 [(M+H)⁺].

### g) (2R,5RS)-5-(6-Bromo-pyridin-2-yl)-2,5-dimethyl-2-trifluoromethyl-5,6-dihydro-2H-[1,4]oxazin-3-ylamine

A suspension of (R)-2-[(RS)-2-amino-2-(6-bromo-pyridin-2-yl)-propoxy]-3,3,3-trifluoro-2-methyl-propionitrile (0.688 g, 1.172 mmol), N-acetyl-L-cysteine (0.383 g, 2.344 mmol) and K₂CO₃ (0.356 g, 2.560 mmol) in abs. EtOH (4 ml) was stirred at 80° C. for 18 h. The reaction mixture was quenched with 10% aq. K₂CO₃ soln. and 3× extracted with TBDME. The combined org. phases were washed with brine, dried over Na₂SO₄, filtered and concentrated to leave the title compound as a colourless solid. HPLC Rt_{H11}=0.68-0.70 min; ESIMS: 352, 354 [(M+H)⁺].

### h) [(2R,5R)-5-(6-Bromo-pyridin-2-yl)-2,5-dimethyl-2-trifluoromethyl-5,6-dihydro-2H-[1,4]oxazin-3-yl]-carbamic acid tert-butyl ester and (2R,5S)-diastereomer

A mixture of (R)-5-(6-Bromo-pyridin-2-yl)-2,5-dimethyl-2-trifluoromethyl-5,6-dihydro-2H-[1,4]oxazin-3-ylamine, Boc₂O and DIPEA in DCM (4 ml) was stirred at rt for 20 h. The reaction mixture was quenched with sat. aq. NaHCO₃ soln. and diluted with DCM. The phases were separated and the aq. phase was twice reextracted with DCM. The combined org. phases were washed with brine, dried over Na₂SO₄, filtered and concentrated. HPLC purification (Alltech Grom Saphir 65 Si, 10 μm, 250×50 mm column, gradient Hept:EtOAc 0-1.6 min 85:15, 1.6-16 min 0:100, 16-21.2 min 0:100, flow: 100 ml/min, detection: 254 nm) yielded the desired (2R,5R) as well as the undesired (2R,5S) diastereomer. HPLC Rt_{H11}=1.28 min (2R,5S), 1.30 min (2R,5R); ESIMS: 452, 454 [(M+H)⁺]; ¹H NMR (2R,5R) (400 MHz, CDCl₃): δ 10.98 (br s, 1H), 7.59 (t, 1H), 7.43 (d, 1H), 7.34 (d, 1H), 4.39 (d, 1H), 4.08 (d, 1H), 1.62 (s, 3H), 1.55 (s, 12H); ¹H NMR (2R,5S) (400 MHz, CDCl₃): δ 11.01 (br s, 1H), 7.57 (t, 1H), 7.42 (d, 1H), 7.37 (d, 1H), 4.45 (d, 1H), 3.91 (d, 1H), 1.74 (s, 3H), 1.65 (s, 3H), 1.55 (s, 19H).

### i) ((2R,5R)-5-{6-[(5-Cyano-3-methyl-pyridine-2-carbonyl)-amino]-pyridin-2-yl}-2,5-dimethyl-2-trifluoromethyl-5,6-dihydro-2H-[1,4]oxazin-3-yl)-carbamic acid tert-butyl ester

A mixture of [(2R,5R)-5-(6-Bromo-pyridin-2-yl)-2,5-dimethyl-2-trifluoromethyl-5,6-dihydro-2H-[1,4]oxazin-3-yl]-carbamic acid tert-butyl ester (60.00 mg, 0.133 mmol), 5-cyano-3-methylpicolinamide (23.52 mg, 0.146 mmol), Xantphos (6.91 mg, 0.012 mmol) and Cs₂CO₃ (60.50 mg, 0.186 mmol) in dioxane (0.611 ml) was degassed with argon for 5 min, then Pd₂ dba₃ (3.64 mg, 3.98 μmol) was added and the reaction mixture was stirred at 40° C. for 18 h. The reaction mixture was diluted with H₂O and TBDME. The phases were separated and the aq. phase was reextracted with TBDME. The combined org. phases were washed with brine, dried over Na₂SO₄, filtered and concentrated. HPLC purification (Alltech Grom Saphir 65 Si 10 μM column, 150×30 mm, gradient n-heptane:EtOAc 0-1.2 min 75:25, 1.2-9 min 0:100, 9-12 min 0:100, flow: 50 ml/min, detection: 254 nm) yielded the title compound as a colourless solid. HPLC Rt_{Hii}=1.37 min; ESIMS: 533 [(M+H)⁺]; ¹H NMR (400 MHz, CDCl₃): δ 11.22 (s, 1H), 10.47 (s, 1H), 8.78 (d, 1H), 8.33 (d, 1H), 7.98 (d, 1H), 7.84-7.80 (m, 1H), 7.13 (d, 1H), 4.37 (d, 1H), 4.11 (d, 1H), 2.88 (s, 3H), 1.64 (s, 3H), 1.57 (br s, 12H).

### j) 5-Cyano-3-methyl-pyridine-2-carboxylic acid [64(3R,6R)-5-amino-3,6-dimethyl-6-trifluoro-methyl-3,6-dihydro-2H-[1,4]oxazin-3-yl)-pyridin-2-yl]-amide

To a solution of ((2R,5R)-5-{6-[(5-cyano-3-methyl-pyridine-2-carbonyl)-amino]-pyridin-2-yl}-2,5-dimethyl-2-trifluoromethyl-5,6-dihydro-2H-[1,4]oxazin-3-yl)-carbamic acid tert-butyl ester (50.0 mg, 0.094 mmol) in DCM (0.3 ml) was added TFA (0.289 ml, 428.0 mg, 3.760 mmol) and the reaction mixture was stirred at rt for 2 h. The solvent was evaporated off, sat. aq. NaHCO₃ soln. and TBDME was added, the phases were separated and the aq. phase was reextracted twice with TBDME. The combined org. phases were dried over Na₂SO₄, filtered and concentrated and the residue was washed with MeOH to leave the title compound as a colourless crystalline solid. HPLC Rt_{H11}=0.84 min; ESIMS: 433 [(M+H)⁺]; ¹H NMR (400 MHz, CD₃OD): δ 8.85 (s, 1H), 8.21-8.18 (m, 2H), 7.82-7.78 (m, 1H), 7.23 (d, 1H), 4.18 (d, 1H), 3.80 (d, 1H), 2.76 (s, 3H), 1.46-1.45 (2s, 6H).

### Examples 265 and 266

The compounds listed in Table 28 can be prepared by a procedure analogous to that used in Example 264.

Hydrochlorid salts were obtained from solutions of the corresponding free base by addition of hydrochloric acid in dioxane or hydrochloric acid in diethylether and evaporation of the solvents.

TABLE 28

| | | | MS |
|---|---|---|---|
| | | | [m/z; |
| Example | Compound | ¹H-NMR | (M + 1)⁺] |
| 265 | 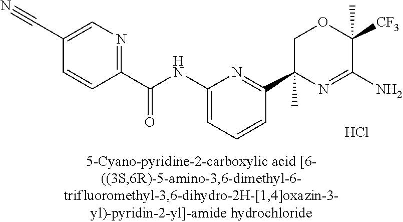 [SMILES: C[C@]1(c2cccc(NC(=O)c3ccc(C#N)cn3)n2)CO[C@@](C)(C(F)(F)F)C(N)=N1 ; Cl] | (δ; CDCl₃): 10.26 (br s, 1H), 8.97 (br s, 1H), 8.45 (d, 1H), 8.29-8.12 (m, 2H), 7.78-7.76 (m, 1H), 7.38 (d, 1H), 4.14 (d, 1H), 3.94 (d, 1H), 1.69 (br s, 3H), 1.55 (br s, 3H). | 419 |
| 266 | 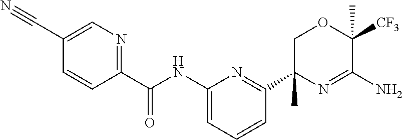 [SMILES: C[C@@]1(c2cccc(NC(=O)c3ccc(C#N)cn3)n2)CO[C@@](C)(C(F)(F)F)C(N)=N1] | (δ; CD₃OD): 9.07 (d, 1H), 8.45-8.39 (m, 2H), 8.22 (d, 1H), 7.83 (t, 1H), 7.27 (d, 1H), 4.19 (d, 1H), 3.83 (d, 1H), 1.47 (s, 6H). | 419 |
| | 5-Cyano-pyridine-2-carboxylic acid [6- | | |
| | ((3R,6R)-5-amino-3,6-dimethyl-6- | | |
| | trifluoromethyl-3,6-dihydro-2H-[1,4]oxazin-3- | | |
| | yl)-pyridin-2-yl]-amide | | |

### Example 267

### 5-Cyano-3-methyl-pyridine-2-carboxylic acid [6-((3R,6R)-5-amino-3,6-dimethyl-6-trifluoromethyl-3,6-dihydro-2H-[1,4]oxazin-3-yl)-5-fluoro-pyridin-2-yl]-amide

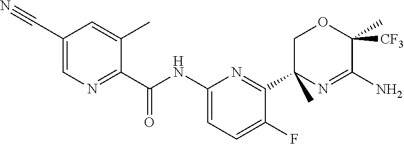 [SMILES: Cc1cc(C#N)cnc1C(=O)Nc1ccc(F)c([C@]2(C)CO[C@@](C)(C(F)(F)F)C(N)=N2)n1]

### a) 2-(6-Bromo-3-fluoro-pyridin-2-yl)-propan-2-ol

To a solution of 2-bromo-5-fluoropyridine (25 g, 142 mmol) in diethylether (600 ml) was slowly added n-butyllithium (2.5 M in hexane, 56.8 ml, 142 mmol) at −78° C. under a nitrogen atmosphere. The resulting yellow reaction mixture was stirred at −78° C. for 2 hours and dry acetone (11.47 ml, 156 mmol) was added over 30 minutes. Stirring was continued at −78° C. for 1 hour. HCl (2N, 50 ml) was added and the reaction mixture was warmed to 0° C. The pH of the mixture was adjusted to ˜7 with 2N HCl solution. The reaction mixture was diluted with ethyl acetate and washed with brine, dried over sodium sulfate, filtered and concentrated in vacuo. The crude product (29.36 g) was chromatographed over silica gel (cyclohexane:ethyl acetate 9:1): 22.3 g (67.1% yield). TLC Rf=0.33 (9:1 cyclohexane:ethyl acetate), LC-MS_{SQ22} Rₜ=0.89 min (ES+ 234, 236). ¹H-NMR (360 MHz, DMSO-d₆): 7.72-7.62 (m, 2H), 5.27 (s, 1H, OH), 1.50 (s, 6H, 2×CH₃).

### b) 6-Bromo-3-fluoro-2-isopropenyl-pyridine

To a solution of 2-(6-bromo-3-fluoro-pyridin-2-yl)-propan-2-ol (22.3 g, 95 mmol) and methanesulfonic acid anhydride (49.8 g, 286 mmol) in dichloromethane was added dropwise triethylamine (53.1 ml, 381 mmol) at 0° C. The reaction mixture was stirred at room temperature for 20 hours. The reaction mixture was quenched with aq. sodium carbonate solution and diluted with dichloromethane. The aqueous phase was extracted with dichloromethane. The combined organic layers were washed with brine, dried over sodium sulfate, filtered and evaporated in vacuo (volatile). The crude brown oil was chromatographed over silica (cyclohexane:ethyl acetate 9:1) to give the title compound as a clear liquid. 17.35 g (84% yield). TLC Rf=0.58 (9:1 cyclohexane:ethyl acetate). ¹H-NMR (360 MHz, CDCl₃): 7.26-7.15 (m, 2H), 5.72 (s, 1H), 5.47 (s, 1H), 2.12 (s, 3H, CH₃).

### c) 2-(6-Bromo-3-fluoro-pyridin-2-yl)-propane-1,2-diol

To a solution of 6-bromo-3-fluoro-2-isopropenyl-pyridine (17.35 g, 80 mmol) in acetone (45 ml) and water (90 ml) was added N-methylmorpholine-N-oxide hydrate (11.4 g, 84 mmol) and osmium tetroxide (5.04 ml, 0.402 mmol). The resulting reaction mixture was stirred at room temperature for 44 hours. Sodium dithionite (2 g) in water (70 ml) was added and the reaction mixture was stirred for 15 minutes and was then filtered and concentrated in vacuo. Ethyl acetate was added and the organic layer was washed with brine, dried over sodium sulfate, filtered and evaporated. 18.29 g slightly yellow solid (91% yield). LC-MS_{SQ22} Rₜ=0.64 min (ES+ 250, 252). ¹H-NMR (360 MHz, CDCl₃): 7.46 (dd, 1H), 7.35 (dd, 1H), 5.09 (s, 1H, OH), 3.96 (d, 1H), 3.78 (d, 1H), 2.45 (broad, 1H, OH), 1.53 (s, 3H, CH₃).

### d) Methanesulfonic acid 2-(6-bromo-3-fluoro-pyridin-2-yl)-2-hydroxy-propyl ester

To a solution of 2-(6-bromo-3-fluoro-pyridin-2-yl)-propane-1,2-diol (18.29 g, 73.1 mmol) in dichloromethane (350 ml) was added triethylamine (20.39 ml, 146 mmol). Methanesulfonyl chloride (6.27 ml, 80 mmol) was added dropwise at 0° C. over 10 minutes. Stirring was continued at 0° C. for 30 minutes. The reaction mixture was washed with sat. sodium bicarbonate solution, water and brine. The organic layer was dried over sodium sulfate, filtered and evaporated. 31.46 g (crude, used without further purification in the next step). LC-MS Rt_{SQ22}=0.81 min. (ES+ 328, 330). ¹H-NMR (360 MHz, CDCl₃): 7.52 (dd, 1H), 7.41 (dd, 1H), 5.13 (s, 1H, OH), 4.61 (d, 1H), 4.45 (d, 1H), 3.05 (s, 3H, CH₃SO₂), 1.61 (s, 3H, CH₃).

### e) 1-Azido-2-(6-bromo-3-fluoro-pyridin-2-yl)-propan-2-ol

A mixture of methanesulfonic acid 2-(6-bromo-3-fluoro-pyridin-2-yl)-2-hydroxy-propyl ester (5 g, 15.24 mmol), ammonium chloride (4.08 g, 76 mmol) and sodium azide (2.476 g, 38.1 mmol) in ethanol (100 ml) was stirred at 80° C. for 20 hours. The reaction mixture was diluted with ethyl acetate and washed with water and brine. The organic layer was dried over sodium sulfate, filtered and evaporated. 3.1 g (74% yield). TLC Rf=0.35 (9:1 cyclohexane:ethyl acetate), LC-MS Rt_{SQ22}=0.97 min. (ES+ 275, 277). ¹H-NMR (360 MHz, CDCl₃): 7.51 (dd, 1H), 7.36 (dd, 1H), 5.18 (s broad, 1H, OH), 3.68-3.60 (AB system, 2H), 1.59 (s, 3H, CH₃).

### f) 6-Bromo-3-fluoro-2-(2-methyl-aziridin-2-yl)-pyridine

To a solution of 1-azido-2-(6-bromo-3-fluoro-pyridin-2-yl)-propan-2-ol (11.2 g, 40.7 mmol) in THF (60 ml) was added triphenylphosphine (10.68 g, 40.7 mmol) and the reaction mixture was stirred for 18 hours at room temperature. The solvent was removed in vacuo and the residue obtained was dissolved in diethylether and filtered through a cotton plug to remove triphenylphosphine oxide. The filtrate was washed with citric acid (9.6 g in 20 ml of water) and the organic phase was separated. The aqueous layer was made basic with 2N NaOH and extracted with diethylether. The organic layer was dried over sodium sulfate, filtered and evaporated to yield the title compound with some TPPO present: 8.1 g yellow oil (69% yield). TLC Rf=0.28 (2:1 cyclohexane:ethyl acetate). LC-MS Rt_{ZQ01}=0.46. (ES+ 231, 233)

¹H-NMR (400 MHz, CDCl₃): 7.34 (dd, 1H), 7.24 (dd, 1H), 1.99 (s, 1H), 1.89 (s, 1H), 1.65 (s, 3H, CH₃).

### g) 6-Bromo-3-fluoro-2-[2-methyl-1-(2-nitro-benzenesulfonyl)-aziridin-2-yl]-pyridine

To a solution of 6-bromo-3-fluoro-2-(2-methyl-aziridin-2-yl)-pyridine (8 g, 27.7 mmol) in THF (48 ml) and water (16 ml) was added N-methylmorpholine (3.5 ml, 27.7 mmol) and o-nosylchloride. The reaction mixture was stirred for 4 hours at room temperature. 3 g neutral Alox was added and the reaction mixture was filtered. The filtrate was diluted with dichloromethane, washed with sat. sodium hydrogencarbonate solution and water. The organic phase was dried over sodium sulfate, filtered and evaporated. 11.2 g of the crude product was purified over silica gel (cyclohexane:ethyl acetate 60:40) to afford the title compound. 8.69 g (75% yield). LC-MS Rt_{SQ22}=1.09 min. (ES+ 416, 418). ¹H-NMR (400 MHz, CDCl₃): 8.27 (m, 1H), 7.80-7.73 (m, 3H), 7.46 (dd, 1H), 7.34 (dd, 1H), 3.32 (s, 1H), 3.20 (s, 1H), 2.10 (s, 3H, CH₃).

### h) (R)-2-[2-(6-Bromo-3-fluoro-pyridin-2-yl)-2-(2-nitro-benzenesulfonylamino)-propoxy]3,3-trifluoro-2-methyl-propionic acid ethyl ester

To a solution of (R)-3,3,3-Trifluoro-2-hydroxy-2-methyl-propionic acid ethyl ester (715 mg, 3.84 mmol) in DMF (4 ml) was added NaH (55%) (154 mg, 3.84 mmol) at room temperature and the reaction mixture was stirred for 30 minutes at room temperature. A solution of 6-bromo-3-fluoro-2-[2-methyl-1-(2-nitro-benzenesulfonyl)-aziridin-2-yl]-pyridine (800 mg, 1.922 mmol) in DMF (9 ml) was added and the reaction mixture was stirred at room temperature for 48 hours. The reaction mixture was poured onto a mixture of ice/2N HCl/t-butyl-methylether. The organic layer was washed with sat. sodium bicarbonate solution and brine, dried over sodium sulfate, filtered and evaporated. Silica gel chromatography (cyclohexane/ethyl acetate) afforded the title compound as a mixture of 2 diastereoisomers. 300 mg (26% yield). TLC Rf=0.42 (cyclohexane:ethyl acetate 2:1). LC-MS Rt_{SQ22}=1.25 min. (100%, TIC ES+ 602, 604)

### i) (R)-2-[2-(6-Bromo-3-fluoro-pyridin-2-yl)-2-(2-nitro-benzenesulfonylamino)-propoxy]-3,3,3-trifluoro-2-methyl-propionamide

A solution of (R)-2-[2-(6-bromo-3-fluoro-pyridin-2-yl)-2-(2-nitro-benzenesulfonylamino)-propoxy]3,3,3-trifluoro-2-methyl-propionic acid ethyl ester (720 mg, 1.195 mmol) in NH3 7N in methanol (19 ml, 133 mmol) was stirred at 50° C. for 2 days in a sealed 25 ml microwave vial. The solvent was removed in vacuo and the residue (987 mg) was chromatographed over silica gel (cyclohexane/ethyl acetate) affording the title compound as a mixture of two diastereoisomers (500 mg, 73% yield). TLC Rf=0.30 (cyclohexane:ethyl acetate 1:1). LC-MS_{SQ22} Rt=1.05 min (ES+ 573, 575).

### j) N-[1-(6-Bromo-3-fluoro-pyridin-2-yl)-2-((R)-1-cyano-2,2,2-trifluoro-1-methyl-ethoxy)-1-methyl-ethyl]-2-nitro-benzenesulfonamide

To a solution of (R)-2-[2-(6-Bromo-3-fluoro-pyridin-2-yl)-2-(2-nitro-benzenesulfonylamino)-propoxy]-3,3,3-trifluoro-2-methyl-propionamide (200 mg, 0.349 mmol) and triethylamine (0.121 ml, 0.872 mmol) in dichloromethane (3 ml) was added TFAA (0.059 ml, 0.419 mmol) at 0-5° C. and the reaction mixture was stirred for 18 hours at room temperature. Further addition of TFFA and triethylamine (0.6 and 1.2 aequivalent, respectively) brought the reaction to completion after 24 hours. The reaction mixture was added to a cold sat. sodium bicarbonate solution and the product was extracted with dichloromethane. The organic layer was washed with cold 0.1 N HCl solution, water and sat. sodium bicarbonate solution, dried over sodium sulfate, filtered and evaporated in vacuo. 190 mg (98% yield) crude product as a mixture of 2 diastereiosomers. TLC R_{f}=0.24 (cyclohexane:ethyl acetate 3:1), LC-MS Rt_{SQ22}=1.20 min. (ESI+555, 557).

### k) (2R,5S)-5-(6-Bromo-3-fluoro-pyridin-2-yl)-2,5-dimethyl-2-trifluoromethyl-5,6-dihydro-2H-[1,4]oxazin-3-ylamine and (2R,5R)-5-(6-Bromo-3-fluoro-pyridin-2-yl)-2,5-dimethyl-2-trifluoromethyl-5,6-dihydro-2H-[1,4]oxazin-3-ylamine

A solution of N—[1-(6-Bromo-3-fluoro-pyridin-2-yl)-2-((R)-1-cyano-2,2,2-trifluoro-1-methyl-ethoxy)-1-methyl-ethyl]-2-nitro-benzenesulfonamide (1000 mg, 1.801 mmol), potassium carbonate (548 mg, 3.96 mmol) and N-acetylcysteine (588 mg, 3.6 mmol) in ethanol (17 ml) was stirred at 80° C. for 3 days until all starting material was consumed. The reaction mixture was concentrated in vacuo and the yellow foam redissolved in ethyl acetate and 20% aqueous potassium carbonate solution. The organic phase was washed with sat. sodium bicarbonate solution and brine, dried over magnesium sulfate, filtered and evaporated. 660 mg yellow oil. The 2 diastereoisomers were separated via normal phase preparative HPLC chromatography (cychlohexane/ethyl acete/MeOH). (2R,5S)-5-(6-Bromo-3-fluoro-pyridin-2-yl)-2,5-dimethyl-2-trifluoromethyl-5,6-dihydro-2H-[1,4]oxazin-3-ylamine (cis derivative): 76 mg. TLC R_{f}=0.26 (toluene:ethyl acetate 8:2+5% ETA). LC-MS Rt_{SQ12}=0.73 min (100% purity, El+ 370, 372). ¹H-NMR (600 MHz, DMSO-D₆): 7.69-7.61 (m, 2H), 6.0 (broad s, 2H, NH2, amidine), 4.15 (d, 1H, AB-system), 3.71 (s, 1H, AB-system), 1.59 (s, 3H, CH₃), 1.47 (s, 3H, CH₃). (2R,5R)-5-(6-Bromo-3-fluoro-pyridin-2-yl)-2,5-dimethyl-2-trifluoromethyl-5,6-dihydro-2H-[1,4]oxazin-3-ylamine (trans derivative): 89 mg. TLC Rf=0.31 (toluene:ethyl acetate 8:2+5% ETA). LC-MS Rt_{SQ12}=0.73 min (100% purity, El+ 370, 372). ¹H-NMR (600 MHz, DMSO-D₆): 7.73-7.61 (m, 2H), 6.0 (broad s, 2H, NH₂, amidine), 4.04 (d, 1H, AB-system), 3.72 (d, 1H, AB-system), 1.52 (s, 3H, CH₃), 1.48 (s, 3H, CH₃).

### l) 5-Cyano-3-methyl-pyridine-2-carboxylic acid [64(3R,6R)-5-amino-3,6-dimethyl-6-trifluoromethyl-3,6-dihydro-2H-[1,4]oxazin-3-yl)-5-fluoro-pyridin-2-yl]-amide

A mixture of (2R,5R)-5-(6-bromo-3-fluoro-pyridin-2-yl)-2,5-dimethyl-2-trifluoromethyl-5,6-dihydro-2H-[1,4]oxazin-3-ylamine (80 mg, 0.216 mmol), 5-cyano-3-methyl-pyridine-2-carboxylic acid amide (34.8 mg, 0.216 mmol, see Intermediates Amide 1), xantphos (11.26 mg, 0.019 mmol) and cesium carbonate (99 mg, 0.303 mmol) in dioxane (2 ml) was degassed for 5 minutes with argon. Pd₂(dba)₃ (5.94 mg, 6.48 μmol) was added, the microwave vial was sealed and stirred at 80° C. for 18 hours. The reaction mixture was diluted with water and TBDME. The organic phase was washed with brine, dried over sodium sulfate, filtered and evaporated. 173 mg orange solid. Silica gel chromatography (applied on two 20×20 cm plate, 1 mm, dichloromethane:methanol 9:1, rechromatographed with dichloromethane:methanol 95:5 with double evolution of the plates) afforded the title compound: 15 mg and 21 mg. Combined amount: 36 mg (37% yield). TLC R_{f}=0.53 (dichloromethane:methanol 9:1). API ES+ MS 451. LC-MS Rt_{SQ01}=0.87 min. (100%, ES+ 451). ¹H-NMR (400 MHz, CDCl₃): 10.80 (br s, 1H), 8.83 (br s, 1H), 8.41 (dd, 1H), 7.93 (br s, 1H), 7.55 (t, 1H), 5.8-4.6 (very broad, 2H), 4.23 (br s, 2H), 2.83 (s, 3H), 1.75 (s, 3H), 1.66 (s, 3H).

### Example 268

### 5-Cyano-3-methyl-pyridine-2-carboxylic acid [6-((3S,6R)-5-amino-3,6-dimethyl-6-trifluoromethyl-3,6-dihydro-2H-[1,4]oxazin-3-yl)-5-fluoro-pyridin-2-yl]-amide

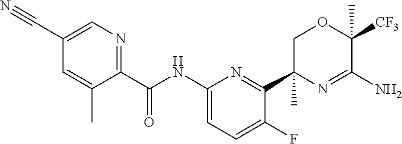 [SMILES: Cc1cc(C#N)cnc1C(=O)Nc1ccc(F)c([C@@]2(C)CO[C@@](C)(C(F)(F)F)C(N)=N2)n1]

5-Cyano-3-methyl-pyridine-2-carboxylic acid [64(3S,6R)-5-amino-3,6-dimethyl-6-trifluoromethyl-3,6-dihydro-2H-[1,4]oxazin-3-yl)-5-fluoro-pyridin-2-yl]-amide can be prepared by a procedure analogous to that used in Example 267.

TLC R_{f}=0.47 (dichloromethane:methanol 9:1). API ES+ MS 451. LC-MS Rt_{SQ01}=0.86 min. (100%, ES+ 451). ¹H-NMR (400 MHz, CDCl₃): 10.65 (br s, 1H), 8.83 (d, 1H), 8.37 (dd, 1H), 7.96 (d, 1H), 7.51 (dd, 1H), 6.0-5.0 (very broad, 2H), 4.38 (d, 1H), 4.09 (d, 1H), 2.85 (s, 3H), 1.78 (s, 3H), 1.71 (s, 3H).

### Example 269

### 5-{2-[(5-Chloro-4,6-dideutero-3-trideuteromethyl-pyridine-2-carbonyl)-amino]-pyridin-4-yl}-5-fluoromethyl-5,6-dihydro-2H-[1,4]oxazin-3-yl-ammonium acetate

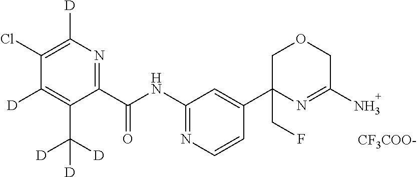

### a) 2-(2-Bromo-pyridin-4-yl)-malonic acid diethyl ester

2-Bromo-4-methylpyridine (70.0 g, 407 mmol) was added drop wise to a cooled (−78° C.) solution of LDA (2.0 M in toluene/THF/ethyl benzene, 610.4 ml, 1.22 mol) in dry THF (600 ml) for 30 min. ethylchloroformate (132.3 g, 1.22 mol) was added to the resultant reaction mixture with addition funnel at −78° C. and stirring continued for 90 min. Reaction mixture was treated with saturated NH₄Cl solution and worked up with ethyl acetate by washing with water, brine followed by drying over anhy. Na₂SO₄. Organic layer was concentrated under reduced pressure to obtain crude product which was purified by column chromatography with 10% ethyl acetate in Hexane to furnish title compound as a brown color oily liquid. Yield: 115.0 g (89%). TLC (10% ethyl acetate in hexane: Rf=0.15). LCMS: Rt_{H9}=1.475 [M+1]⁺=315.8 and 317.8; HPLC Rt_{H15}=7.30 min (86.7%); ¹H NMR (400 MHz, CDCl₃) δ 8.38 (d, 1H), 7.56 (t, 1H), 7.34 (dd, 1H), 4.55 (s, 1H), 4.29-4.18 (m, 4H), 1.28 (t, 6H).

### b) (2-Bromo-pyridin-4-yl)-acetic acid

A suspension of 2-(2-bromo-pyridin-4-yl)-malonic acid diethyl ester (115 g, 316 mmol) and K₂CO₃ (125.23 g, 907.5 mmol) in water (500 ml) was heated at 100° C. for 8 h under constant stirring. Reaction mixture was cooled to rt and concentrated under reduced pressure to remove solvent completely. The solid residue was dissolved in minimum quantity of water (25 ml) and washed with 20% ethyl acetate in hexane to remove non polar impurities. The aqueous layer was separated and cooled to 0° C. followed by adjusting pH ˜6 to 7 using aq. 6 N HCl. The precipitated solid was filtered using buchner funnel, washed with ice cold water and dried under vacuum to furnish title compound as an off white solid. with sufficient purity. Yield: 60.0 g (76.3%). TLC (70% ethyl acetate in hexane: Rf=0.05).

LCMS: Rt_{H9}=0.193; [M+1]⁺=215.9 and 217.9; HPLC Rt_{H15}=3.025 min (98%); ¹H NMR (400 MHz, CDCl₃): δ 12.71 (s, 1H), 8.33 (d, 1H), 7.61 (s, 1H), 7.37 (d, 1H), 3.71 (s, 3H).

### c) (2-Bromo-pyridin-4-yl)-acetic acid ethyl ester

To a solution of (2-bromo-pyridin-4-yl)-acetic acid (60.0 g, 277.7 mmol) in ethanol (600 ml), conc: sulfuric acid (5.0 ml) was added at rt and the reaction mixture was heated at 90° C. for 9 h. Reaction mixture was cooled to rt and concentrated under reduced pressure to remove solvent completely. The residue obtained was cooled to 0° C. and pH was adjusted to 8 using 10% aqueous NaHCO₃ solution. The resultant contents were worked up with ethyl acetate by washing with water, brine and dried over anhy. Na₂SO₄. Organic layer was concentrated under reduced pressure to obtain crude compound. Column chromtography purification of the crude compound using 15% ethyl acetate in hexane as eluent furnished title compound as a brown oil. Yield: 65.0 g (88.5%). TLC (30% ethyl acetate in hexane: Rf=0.39). LCMS: Rt_{H8}=0.824 [M+1]⁺=243.8 and 245.8; HPLC Rt_{H15}=3.759 min (69%); ¹H NMR (300 MHz, CDCl₃): δ 8.32 (t, 1H), 7.43 (s, 1H), 7.21-7.15 (M, 1H), 4.18 (q, 2H), 1.27 (t, 3H).

### d) 2-(2-Bromo-pyridin-4-yl)-3-hydroxy-2-hydroxymethyl-propionic acid ethyl ester

To an ice cooled stirred mixture of (2-bromo-pyridin-4-yl)-acetic acid ethyl ester (40.0 g, 163.93 mmol) and paraformaldehyde (9.84 g, 327.8 mmol) in dry DCM was added 1,8-diazabicyclo[5.4.0]undec-7-ene (1.49 g, 1.49 ml, 9.83 mmol) and stirred for 2 h. Reaction mixture was treated with (1R)-(−)-10-camphor sulphonic acid (2.283 g, 9.83 mmol) at 0° C. and the organic layer was washed with brine and dried over anhydrous Na₂SO₄. Concentration of organic layer afforded gummy oily material. The crude compound was purified over triethyl amine treated silicagel using 5%-8% methanol in DCM as eluent furnished title compound as a brown liquid. Yield=20.0 g (40%). TLC (30% ethyl acetate in hexane: Rf=0.06). LCMS: Rt_{H8}=0.191; [M+1]⁺=303.9 and 305.8; HPLC Rt_{H15}=6.019 min (43%); ¹H NMR (400 MHz, CDCl₃): δ 8.36-8.31 (m, 1H), 7.34-7.25 (m, 1H), 6.37-6.33 (m, 1H), 4.6 (d, 1H), 4.14-4.08 (m, 2H), 4.04-3.91 (m, 4H), 1.13 (t, 3H).

### e) 5-(2-Bromo-pyridin-4-yl)-2,2-dimethyl-[1,3]dioxane-5-carboxylic acid ethyl ester

A mixture of 2-(2-bromo-pyridin-4-yl)-3-hydroxy-2-hydroxymethyl-propionic acid ethyl ester (30.0 g, 98.6 mmol) 2,2-dimethoxy propane (51.11 g, [60.5 ml], 493.1 mmol) and (1R)-(−)-10-camphor sulphonic acid (5.72 g, 24.65 mmol) in DMF (100 ml) was heated at 80° C. for 10 h. Reaction mixture was cooled to rt and concentrated under reduced pressure. The residue was dissolved in ethyl acetate and worked up by washing with water, brine, followed by drying over anhy. Na₂SO₄. Organic layer was concentrated under reduced pressure and the crude product was purified by column chromatography using 10% ethyl acetate in Hexane to obtain title compound as a yellow solid. Yield=18.15 g (53%). TLC (30% ethyl acetate in hexane: Rf=0.52). LCMS: Rt_{H8}=1.487; [M+l]⁺=344.0 and 346.0; HPLC Rt_{H15}=7.6 min (74%); ¹H NMR (300 MHz, CDCl₃): δ 8.41-8.34 (t, 1H), 7.54 (s, 1H), 7.32-7.28 (m, 1H), 4.51 (dd, 2H), 4.27-4.21 (q, 4H), 1.45 (s, 3H), 1.39 (s, 3H), 1.23 (t, 3H).

### f) 5-(2-Bromo-pyridin-4-yl)-2,2-dimethyl-[1,3]dioxane-5-carboxylic acid ethyl ester

A solution of LiOH.H₂O (11.1 g, 263.5 mmol) in water (10 ml) was added to a solution of 5-(2-bromo-pyridin-4-yl)-2,2-dimethyl-[1,3]dioxane-5-carboxylic acid ethyl ester (18.1 g, 52.7 mmol) in ethanol (60 ml) at 0° C. and the resultant reaction mixture was stirred at rt for 3 h. Reaction mixture was concentrated under reduced pressure to remove solvent completely. The wet mass obtained was cooled to 0° C., acidified with glacial acetic acid (to maintain pH ˜6) and the product was extracted with ethyl acetate (2×100 ml). Organic layer was washed with brine and concentrated to afford brown solid which was used in the next step without further purification. Yield=14.1 g (85%). TLC (50% ethyl acetate in hexane: Rf=0.03). LCMS: Rt_{H9}=0.343 [M+1]⁺=316.0, 318.0; ¹H NMR (300 MHz, CDCl₃): δ 8.28-8.21 (t, 1H), 7.7 (s, 1H), 7.58-7.54 (m, 1H), 4.21-3.95 (dd, 4H), 1.36 (s, 3H), 1.14 (s, 3H).

### g) 5-(2-Bromo-pyridin-4-yl)-2,2-dimethyl-[1,3]dioxan-5-ylamine

Dipheny phosphoryl azide (14.3 mL, 66.45 mmol) was added to a solution of 5-(2-bromo-pyridin-4-yl)-2,2-dimethyl-[1,3]dioxane-5-carboxylic acid ethyl ester (14.0 g, 44.3 mmol) and triethyl amine (17.24 ml, 133.0 mmol) in toluene (100 ml) at 0° C. The resultant reaction mixture was heated to 80° C. under constant stirring for 7 h. Reaction mixture was concentrated under reduced pressure to remove solvent completely. The residue obtained after concentration was dissolved in THF (100 ml) and cooled to 0° C. 2 N aq. NaOH solution was added drop wise and stirred for 30 min at rt. Reaction mixture was concentrated under reduced pressure to remove THF and the residue obtained was extracted with ethylacetate. Organic layer was washed with water, brine and dried over anhy. Na₂SO₄. Organic layer was concentrated under reduced pressure to obtain furnished brownish oily material which was solidified at low temperature (<10° C.). Yield=9.5 g (75%). TLC (50% ethyl acetate in hexane: Rf=0.15). LCMS: Rt_{H8}=0.083; [M+1]⁺=287.0 and 289.0.

### h) N-[5-(2-Bromo-pyridin-4-yl)-2,2-dimethyl-[1,3]dioxan-5-yl]-2-chloro-acetamide

To a solution of 5-(2-bromo-pyridin-4-yl)-2,2-dimethyl-[1,3]dioxan-5-ylamine (9.5 g, 33.1 mmol) in DCM (100 ml) was added aq. Na₂CO₃ (8.7 g in 50 ml) at 0° C. and stirring continued for 5 min. Chloroacetyl chloride (2.9 ml, 36.41 mmol) was added to the resultant reaction mixture drop wise and stirred for 30 min at 0° C. Reaction mass was diluted with DCM (200 ml) and organic layer was washed successively washed with water, brine, dried over anhydrous Na₂SO₄ and concentrated under reduced pressure to obtain brown solid. The product was directly used in the next step with further purification. Yield=10.2 g (85%). TLC (50% ethyl acetate in hexane: Rf=0.15). LCMS: Rt_{H9}=0.55 [M+1]⁺=363.0 and 364.9.

### i) N-[1-(2-Bromo-pyridin-4-yl)-2-hydroxy-1-hydroxymethyl-ethyl]-2-chloro-acetamide

A solution of N-[5-(2-bromo-pyridin-4-yl)-2,2-dimethyl-[1,3]dioxan-5-yl]-2-chloro-acetamide (10.0 g, 27.6 mmol) in DCM (150 ml) was cooled to 0° C. for 10 min. and trifluoromethyl acetic acid (15.0 ml) was added. Stirring continued for 2 h and the resultant contents were concentrated under reduced pressure. The residue formed was basified with aq. NH₄OH solution and the product was extracted with ethyl acetate (3×200 ml) by washing organic layer with brine (5.0 ml) and dried over anhy. Na₂SO₄. Organic layer was concentrated under reduced pressure to obtain title compound as a brown liquid which was carried to next step without any purification. Yield=8.1 g (91%). TLC (70% ethyl acetate in hexane: Rf=0.15). LCMS: Rt_{H9}=0.12 [M+1]⁺=322.9 and 324.9; HPLC Rt_{H15}=5.266 min (61%), 5.104 (25%).

### j) 5-(2-Bromo-pyridin-4-yl)-5-hydroxymethyl-morpholin-3-one

To a solution of N-[1-(2-bromo-pyridin-4-yl)-2-hydroxy-1-hydroxymethyl-ethyl]-2-chloro-acetamide (8.0 g, 24.8 mmol) in t-BuOH (50 ml) was added t-BuOK (5.5 g, 49.6 mmol) and NaI (0.375 g, 2.48 mmol) and heated to 90° C. for 1 h. Reaction mass was concentrated under reduced pressure and diluted the residue with EtOAc. Organic layer was separated and washed with ammonium chloride solution, brine followed by drying over anhy. Na₂SO₄. The crude product was purified by column chromatography using 5% methanol in DCM to obtain title compound as a pale brown gum. Yield=3.25 g (46%). TLC (ethyl acetate: Rf=0.17). LCMS: Rt_{H9}=0.12; [M+1]⁺=286.7 and 289.

### k) 5-(2-Bromo-pyridin-4-yl)-5-fluoromethyl-morpholin-3-one

To a suspension of 5-(2-bromo-pyridin-4-yl)-5-hydroxymethyl-morpholin-3-one (3.25 g, 11.0 mmol), Na₂CO₃ (3.5 g, 13.06 mmol) in dry THF (15 ml) was added diethylaminosulfur trifluoride (2.25 ml, 17.0 mmol) at 0° C. The reaction mixture was allowed to warm to rt and stirred for 2 h. Solid Na₂CO₃ (3.5 g) was again added to the reaction mixture and stirred for 4 h at rt. Solids present in the reaction mixture filtered through Buchner funnel. Filtrate was concentrated under reduced pressure and the crude product was purified by column chromatography using 5% methanol in DCM to obtain title compound as a pale yellow solid. Yield=2.1 g (66%). TLC (50% ethyl acetate in hexane: Rf=0.17). LCMS: Rt_{H8}=0.201; [M+1]⁺=289 and 291; HPLC: Rt_{H18}=5.171 min. (50%) and 5.063 (21%).

### l) 5-Chloro-4,6-dideutero-3-trideuteromethyl-pyridine-2-carboxylic acid [4-(3-fluoromethyl-5-oxo-morpholin-3-yl)-pyridin-2-yl]-amide

A stirred solution of 5-(2-bromo-pyridin-4-yl)-5-fluoromethyl-morpholin-3-one (0.2 g, 0.695 mmol), 5-chloro-4,6-dideutero-3-trideuteromethyl-pyridine-2-carboxylic acid (Acid 2) (0.135 g, 0.763 mmol) and cesium carbonate (0.678 g, 2.085 mmol) in 1,4-dioxane (5.0 ml) was degassed with argon for 10 min. 4,5-Bis(diphenyl phosphino)-9,9-dimethyl xanthenes (0.041 g, 0.035 mmol) was added to the resultant mixture and degassed again for 10 min. Tris(dibenzylidene-acetone) di palladium(0) (0.032 g, 0.07 mmol) was then added finally and degassed with argon for further 5 min. Reaction mixture was heated to 80° C. for 20 h and cooled to rt. Water was added to the reaction mixture and product was extracted with ethyl acetate by washing with brine followed by drying over anhy. Na₂SO₄. The organic layer was concentrated under reduced pressure to obtain title compound as a sticky solid which was used for the next step without purification. Yield=0.14 g (52%). TLC (50% ethyl acetate in hexane: Rf=0.45).

LCMS: Rt_{H9}=0.868 [M+1]⁺=384.0; ¹H NMR (300 MHz, CDCl₃): δ 10.7 (s, 1H), 8.51-8.41 (m, H) 7.51-7.46 (d, 1H), 7.34-7.16 (m, 1H), 4.99-4.60 (m, 2H), 4.34-3.79 (m, 4H).

### m) 5-Chloro-4,6-dideutero-3-trideuteromethyl-pyridine-2-carboxylic acid [4-(3-fluoromethyl-5-thioxo-morpholin-3-yl)-pyridin-2-yl]-amide

Lawesson's reagent (0.46 g, 1.135 mmol) was added to a stirred solution of 5-chloro-4,6-dideutero-3-trideuteromethyl-pyridine-2-carboxylic acid [4-(3-fluoromethyl-5-oxo-morpholin-3-yl)-pyridin-2-yl]-amide (0.14 g, 0.378 mmol) in THF (4.0 ml) and heated to reflux for 2 h. The reaction mixture was concentrated under reduced pressure to obtain crude product as a sticky solid which was purified by column chromatography using 25% ethyl acetate in hexane as eluent to obtain title compound as a sticky solid. Yield=0.095 g (65%). TLC (30% ethyl acetate in hexane: Rf=0.61). LCMS: Rt_{H8}=1.489 [M+1]⁺=399.8.

### n) 5-{2-[(5-Chloro-4,6-dideutero-3-trideuteromethyl-pyridine-2-carbonyl)-amino]-pyridin-4-yl}-5-fluoromethyl-5,6-dihydro-2H-[1,4]oxazin-3-yl-ammonium acetate

A solution of 5-chloro-4,6-dideutero-3-trideuteromethyl-pyridine-2-carboxylic acid [4-(3-fluoromethyl-5-thioxo-morpholin-3-yl)-pyridin-2-yl]-amide (0.095 g, 0.238 mmol) in 10% methanolic ammonia (5.0 ml) was stirred in a sealed tube for 16 h at rt. Reaction mixture was concentrated under reduced pressure to obtain semi-solid. Product was purified by preparative HPLC method to obtain title compound as a semi solid. Conditions for Preparative HPLC: Column: Agilent Zorbax XDB C18. Mobile phase: A: 10 mm; ammonium acetate; B: ACN, 60 ml; Flow: 20 ml/min.; Gradient: 0-30, 2-40, 10-80. Yield=28 mg (31%). LCMS: Rt_{H8}=0.191 [M+1]⁺=383.1; HPLC: Rt_{H18} 3.208 min (97%); ¹H NMR (300 MHz, DMSO-d₆): δ 10.52 (s, 1H), 8.35 (dd, 2H), 7.28 (d, 1H), 6.15 (br. s, 1H), 4.51-4.28 (m, 2H), 4.07-3.94 (m, 3H), 3.69 (d, 2H), 1.89 (s, 3H); ¹⁹F NMR (376.1): δ −218.9.

### Examples 270 and 1271

The compounds listed in Table 29 were prepared by a procedure analogous to those used in Example 269.

TABLE 13

| | | | MS |
|---|---|---|---|
| | | ¹H-NMR | [m/z; |
| Example | Compound | (δ; DMSO-d₆) | (M + 1)⁺] |
| 270 | 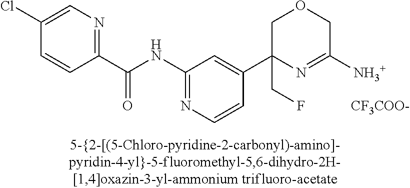 | 10.3 (br. s, 1H), 8.83 (d, 1H, J = 2.0 Hz), 8.45 (s, 1H), 8.33 (d, 1H, J = 5.2 Hz), 8.26-8.18 (m, 2H), 7.31 (d, 1H, J = 5.6 Hz), 6.07 (brs, 2H), 4.51-4.28 (m, 2H), 4.06-3.93 (m, 4H), 3.7-3.67 (m, 2H). | LCMS: Rt_{H8} = 0.112 [M + 1]⁺ = 363.9 |
| 271 | 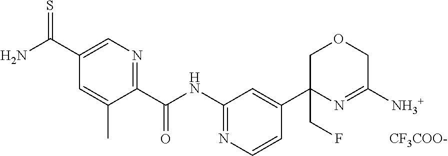 | 8.96 (d, 1H), 8.51 (s, 2H), 8.45 (d, 2H), 8.19 (s, 1H), 7.32 (d, 2H), 5.11-4.99 (m, 2H), 4.72 (s, 3H), 4.27-4.19 (m, 2H), 4.16-4.08 (m, 2H). | LCMS: Rt_{H7} = 0.118; [M + ]⁺ = 403.1 |
| | 5-Fluoromethyl-5-{2-[(3-methyl-5- | | |
| | thiocarbamoyl-pyridine-2-carbonyl)-amino]- | | |
| | pyridin-4-yl}-5,6-dihydro-2H-[1,4]oxazin-3-yl- | | |
| | ammonium trifluoro-acetate | | |

### Example 272

### 5-Cyano-3-methyl-pyridine-2-carboxylic acid [4-((3R,6R)-5-amino-3,6-dimethyl-6-trifluoromethyl-3,6-dihydro-2H-[1,4]oxazin-3-yl)-5-fluoro-pyridin-2-yl]-amide

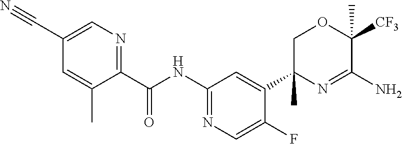 [SMILES: Cc1cc(C#N)cnc1C(=O)Nc1cc([C@]2(C)CO[C@@](C)(C(F)(F)F)C(N)=N2)c(F)cn1]

### a) 2-(2-Bromo-5-fluoro-pyridin-4-yl)-propan-2-ol

To a solution of 2-bromo-5-fluoro-pyridine (CAS 41404-58-4, 25.0 g, 139 mmol) in THF (300 ml) was added dropwise LDA (100 ml of a 2M soln. in THF/heptane/ethylbenzene, 200 mmol) at −78° C. under a N₂ atmosphere. Stirring was continued for 1 h at −78° C., then acetone (20.44 ml, 16.17 g, 278 mmol) was added dropwise and stirring was continued at −78° C. for another 1 h. The reaction mixture was quenched with aq. 1M NH₄Cl soln. and diluted with EtOAc. The phases were separated and the aq. phase was twice reextracted with EtOAc. The combined org. phases were washed with brine, dried over Na₂SO₄, filtered, concentrated. Flash chromatography on silica gel (gradient cyclohexane:EtOAc 100:0 to 90:10) followed by crystallization from pentane yielded the title compound as a colourless solid. HPLC Rt_{H11}=0.81 min; ESIMS: 234, 236 [(M+H)⁺]; ¹H NMR (400 MHz, DMSO-d₆): δ 8.32 (br s, 1H), 7.71 (d, 1H), 5.57 (s, 1H), 4.90 (t, 1H), 3.65-3.57 (m, 1H), 3.53-3.44 (m, 1H), 1.39 (s, 3H).

### b) 2-Bromo-5-fluoro-4-isopropenyl-pyridine

To a solution of 2-(2-bromo-5-fluoro-pyridin-4-yl)-propan-2-ol (24.7 g, 106 mmol) and methanesulfonic anhydride (55.1 g, 317 mmol) in DCM (250 ml) was added triethylamine (58.8 ml, 42.7 g, 422 mmol). The reaction mixture was stirred at rt for 20 h. Another 1 eq. (18 g) of methanesulfonic anhydride and 1.2 eq. (17 ml) of triethylamine were added and the reaction mixture was stirred an additional 20 h at rt. The reaction mixture was quenched with 1M aq. Na₂CO₃ sol. and diluted with DCM. The phases were separated and the aq. phase was reextracted twice with DCM. The combined org. phases were washed with brine, dried over Na₂SO₄, filtered and concentrated. Flash chromatography on silica gel (hexane:EtOAc 8:1) yielded the title compound as a clear colourless liquid. HPLC Rt_{H11}=1.12 min; ESIMS: 216, 218 [(M+H)⁺]; ¹H NMR (400 MHz, CDCl₃): δ 8.20 (d, 1H), 7.40 (d, 1H), 5.48-5.44 (m, 2H), 2.14 (s, 3H).

### c) 2-(2-Bromo-5-fluoro-pyridin-4-yl)-propane-1,2-diol

To a solution of 2-bromo-5-fluoro-4-isopropenyl-pyridine (17.1 g, 79 mmol) in acetone (50 mL) and H₂O (100 mL) was added N-methylmorpholine oxide (10.51 g, 87 mmol) and OsO₄ (4.97 mL, 4.02 g, 0.396 mmol). The biphasic mixture was stirred at rt for 17 h. The reaction mixture was quenched with sodium hydrosulfite (1.516 g, 8.71 mmol) in H₂O (50 ml) and stirred at rt for 20 min. The reaction mixture was filtered through celite and the celite pad was washed three times with acetone. The combined filtrates were evaporated and the residue was taken up with EtOAc and 1N aq. NaOH soln. The phases were separated and the aq. phase was reextracted with EtOAc. The combined org. phases were dried over Na₂SO₄, filtered and concentrated to yield the title compound as a light purple solid. HPLC Rt_{H11}=0.60 min; ESIMS: 250, 252 [(M+H)⁺]; ¹H NMR (400 MHz, DMSO-d₆): δ 8.32 (d, 1H), 7.71 (d, 1H), 5.57 (s, 1H), 4.89 (t, 1H), 3.65-3.57 (m, 1H), 3.53-3.45 (m, 1H), 1.39 (s, 3H).

### d) Methanesulfonic acid 2-(2-bromo-5-fluoro-pyridin-4-yl)-2-hydroxy-propyl ester

To a suspension of 2-(2-bromo-5-fluoro-pyridin-4-yl)-propane-1,2-diol (17.45 g, 69.8 mmol) and triethylamine (19.45 ml, 14.12 g, 140 mmol) in DCM (350 ml) at 0° C. was added dropwise methanesulfonyl chloride (5.71 ml, 8.39 g, 73.3 mmol) over a period of 10 min. The reaction mixture was stirred at 0° C. for 30 min, then quenched with 1M aq. NaHCO₃ soln. The phases were separated, the aq. phase was twice reextracted with DCM and the combined org. phases were washed with brine, dried over Na₂SO₄, filtered and concentrated. Flash chromatography on silica gel (gradient heptane: EtOAc 0-5 min 88:12, 5-37.5 min 24:76) yielded the title compound as a clear oil. HPLC Rt_{H11}=0.76 min; ESIMS: 328, 330 [(M+H)⁺]; ¹H NMR (400 MHz, DMSO-d₆): δ 8.22 (d, 1H), 7.82 (d, 1H), 4.58-4.47 (m, 2H), 3.04 (s, 3H), 3.00 (s, 1H), 1.64 (s, 3H).

### e) 1-Azido-2-(2-bromo-5-fluoro-pyridin-4-yl)-propan-2-ol

To a solution of methanesulfonic acid 2-(2-bromo-5-fluoro-pyridin-4-yl)-2-hydroxy-propyl ester (10.36 g, 31.6 mmol) in ethanol (160 mL) was added NaN₃ (5.13 g, 79.0 mmol) and NH₄Cl (8.44 g, 158.0 mmol). The reaction mixture was stirred at 80° C. for 20 h. The reaction mixture was diluted with H₂O and TBDME and the phases were separated. The aq. phase was twice reextracted with TBDME, the combined org. phases were washed with brine, dried over Na₂SO₄, filtered and concentrated. HPLC Rt_{H11}=0.89 min; ESIMS: 275, 277 [(M+H)⁺]; ¹H NMR (400 MHz, CDCl₃): δ 8.20 (d, 1H), 7.80 (d, 1H), 3.81 (d, 1H), 3.65 (d, 1H), 1.61 (s, 3H).

### f) Methanesulfonic acid 2-azido-1-(2-bromo-5-fluoro-pyridin-4-yl)-1-methyl-ethyl ester

At 0° C., methanesulfonyl chloride (2.04 ml, 3.00 g, 26.20 mmol) was dropwise added to a solution of 1-azido-2-(2-bromo-5-fluoro-pyridin-4-yl)-propan-2-ol (6.00 g, 21.81 mmol) and NEt₃ (3.65 ml, 2.65 g, 26.2 mmol) in DCM (200 ml). The reaction mixture was stirred at 0° C. for 1 h, then for another 1 h at 0° C. to rt. The reaction mixture was quenched with 1M aq. NaHCO₃ soln. and diluted with DCM. The phases were separated and the aq. phase was twice reextracted with DCM. The combined org. phases were dried over Na₂SO₄, filtered and concentrated. HPLC purification (Alltech Grom Saphir 65 Si 10 μM column, 250×50 mm, gradient n-heptane:EtOAc 0-1.6 min 85:15, 1.6-16 min 0:100, 16-21.2 min 0:100, flow 100 ml/min, detection 254 nm) yielded the title compound as well as recovered starting material that could be reacted again according to the above procedure. HPLC Rt_{H11}=0.96 min; ESIMS: 353, 355 [(M+H)⁺]; ¹H NMR (400 MHz, CDCl₃): δ 8.28 (d, 1H), 7.56 (d, 1H), 4.08 (d, 1H), 3.82 (d, 1H), 3.22 (s, 3H), 2.13 (s, 3H).

### g) 2-Bromo-5-fluoro-4-[2-methyl-1-(2-nitro-benzenesulfonyl)-aziridin-2-yl]-pyridine

A mixture of methanesulfonic acid 2-azido-1-(2-bromo-5-fluoro-pyridin-4-yl)-1-methyl-ethyl ester (2.1 g, 6.09 mmol) and PPh₃ (1.597 g, 6.09 mmol) in THF (20 mL) was stirred at rt for 30 min. The reaction mixture was evaporated to dryness, the residue was taken up with TBDME and 10% aq. citric acid soln. The aq. phase was reextracted with TBDME, the combined org. phases were washed with H₂O. The combined aq. phases were basified using 2N aq. NaOH soln. and three times extracted with TBDME. The combined org. phases were dried over Na₂SO₄, filtered and concentrated to yield 2-bromo-5-fluoro-4-(2-methyl-aziridin-2-yl)-pyridine in a mixture with Ph₃PO that was used for the next step without further purification, HPLC Rt_{H11}=0.96 min; ESIMS: 231, 233 [(M+H)⁺].

To a solution of crude 2-bromo-5-fluoro-4-(2-methyl-aziridin-2-yl)-pyridine (3.17 g as a 45% mixture with Ph₃PO, 6.17 mmol) and 2-nitrobenzene-1-sulfonyl chloride (1.368 g, 6.17 mmol) in THF (23.15 mL) and H₂O (7.72 mL) was added N-methylmorpholine and the reaction mixture was stirred at rt for 1.5 h. Alox neutral (2-3 spatula) was added and the reaction mixture was filtered through celite, washed with DCM and the filtrates were diluted with DCM and 1M aq. NaHCO₃ soln. The phases were separated and the aq. phase was reextracted twice with DCM. The combined org. phases were dried over Na₂SO₄ and concentrated. Flash chromatography on silica gel (heptane:EtOAc 4:1 to 3:1) followed by recrystallization from EtOAc/hexane yielded the title compound as a colourless solid. HPLC Rt_{H11}=1.11 min; ESIMS: 416, 418 [(M+H)⁺]; ¹H NMR (400 MHz, CDCl₃): δ 8.31-8.30 (m, 1H), 8.23 (d, 1H), 7.86-7.77 (m, 3H), 7.68 (d, 1H), 3.28 (s, 1H), 2.78 (s, 1H), 2.09 (s, 3H).

### h) (R)-2-[(RS)-2-(2-Bromo-5-fluoro-pyridin-4-yl)-2-(2-nitro-benzenesulfonylamino)-propoxy]-3,3,3-trifluoro-2-methyl-propionic acid ethyl ester

To a solution of 2-bromo-5-fluoro-4-[2-methyl-1-(2-nitro-benzenesulfonyl)-aziridin-2-yl]-pyridine (795 mg, 1.91 mmol) and (R)-3,3,3-trifluoro-2-hydroxy-2-methyl-propionic acid ethyl ester (498 mg, 2.67 mmol) in DMF (8 ml, soln. predried over mol. sieves) was added NaH (99 mg of a 60% dispersion in mineral oil, 2.48 mmol) and the reaction mixture was stirred at rt for 3 h. The reaction mixture was quenched with aq. 1N HCl and diluted with H₂O and TBDME. The phases were separated and the aq. phase was twice extracted with TBDME. The combined org. phases were washed with H₂O, dried over Na₂SO₄, filtered and concentrated. Flash chromatography on silica gel (heptane:EtOAc 1:1) yielded the title compound (diastereomer mixture) as a colourless solid. HPLC Rt_{H11}=1.26 min; ESIMS: 602, 604 [(M+H)⁺]; ¹H NMR (400 MHz, CDCl₃): δ 7.99 (m, 1H), 7.95-7.93 (m, 1H), 7.79-7.61 (m, 4H), 6.94 (m, 1H), 4.45-4.33 (m, 2H), 3.94-3.81 (m, 2H), 1.85 (m, 3H), 1.61 (m, 3H), 1.40-1.34 (m, 3H).

### i) (R)-2-[(RS)-2-(2-Bromo-5-fluoro-pyridin-4-yl)-2-(2-nitro-benzenesulfonylamino)-propoxy]-3,3,3-trifluoro-2-methyl-propionamide

A solution of (R)-2-[(RS)-2-(2-Bromo-5-fluoro-pyridin-4-yl)-2-(2-nitro-benzenesulfonylamino)-propoxy]-3,3,3-trifluoro-2-methyl-propionic acid ethyl ester (920 mg, 1.527 mmol) in 7N NH₃/MeOH (11 ml) was stirred in a sealed glass vial at 55° C. for 44 h. The reaction mixture was evaporated to dryness to leave a yellow solid that was used for the next step without further purification (diastereomer mixture). Rt_{Hii}=1.03 min; ESIMS: 573, 575 [(M+H)⁺]; ¹H NMR (400 MHz, CDCl₃): δ 8.00 (m, 1H), 7.97-7.91 (m, 1H), 7.80-7.63 (m, 3H), 7.55 (m, 1H), 6.63 (m, 1H), 6.41 (m, 1H), 5.74 (m, 1H), 4.15 (m, 1H), 3.97 (m, 1H), 1.84 (2s, 3H), 1.69 (2s, 3H).

### j) N-[(RS)-1-(2-Bromo-5-fluoro-pyridin-4-yl)-2-((R)-1-cyano-2,2,2-trifluoro-1-methyl-ethoxy)-1-methyl-ethyl]-2-nitro-benzenesulfonamide

To a dry solution of (R)-2-[(RS)-2-(2-bromo-5-fluoro-pyridin-4-yl)-2-(2-nitro-benzenesulfonyl-amino)-propoxy]-3,3,3-trifluoro-2-methyl-propionamide (860 mg, 1.35 mmol) in DCM (9 ml) was added at rt NEt₃ (0.470 ml, 342 mg, 3.38 mmol). At 0° C., trifluoroacetic anhydride (0.229 ml, 340 mg, 1.62 ml) was added dropwise. The reaction mixture was allowed to warm to rt and to stir for 1.5 h. The reaction mixture was diluted with 1M aq. Na₂CO₃ soln. and DCM. The phases were separated and the aq. phase was twice reextracted with DCM. The combined org. phases were dried over Na₂SO₄, filtered and concentrated to yield the crude title compound as an orange solid that was used in the next step without further purification (diastereomer mixture). Rt_{H11}=1.19 min; ESIMS: 555, 557 [(M+H)⁺]; ¹H NMR (400 MHz, CDCl₃): δ 8.01-7.93 (m, 2H), 7.79-7.63 (m, 3H), 7.59 (m, 1H), 4.26-4.16 (m, 2H), 1.85-1.84 (2d, 3H), 1.78-1.76 (2d, 3H).

### k) (2R,5R)- and (2R,5S)-5-(2-Bromo-5-fluoro-pyridin-4-yl)-2,5-dimethyl-2-trifluoro-methyl-5,6-dihydro-2H-[1,4]oxazin-3-ylamine

A mixture of N-[1-(2-bromo-5-fluoro-pyridin-4-yl)-2-((R)-1-cyano-2,2,2-trifluoro-1-methyl-ethoxy)-1-methyl-ethyl]-2-nitro-benzenesulfonamide (585 mg, 1.053 mmol), N-acetylcysteine (344 mg, 2.107 mmol) and K₂CO₃ (291 mg, 2.107 mmol) in EtOH (7 ml) was stirred at 85° C. for 68 h under N₂. The reaction mixture was concentrated to ⅓ of its volume and diluted with cold 10% aq. K₂CO₃ soln. and TBDME. The phases were separated and the aq. phase was twice reextracted with TBDME. The combined org. phases were washed with 1M aq. NaHCO₃ soln. and brine, was dried over Na₂SO₄, filtered and concentrated. HPLC purification (Alltech Grom Saphir 65 Si 10 μM column, 150×30 mm, gradient n-heptane:EtOAc:MeOH 0-1.2 min 68:30:2, 1.2-9 min 0:80:20, 9-12 min 0:65:35, flow: 50 ml/min, detection: 254 nm) separated the (2R,5R)- from the (2R,5S)-diastereomer of the title compound. Rt_{H11}=0.70 min; ESIMS: 370, 372 [(M+H)⁺]; ¹H NMR (400 MHz, DMSO-d₆): (2R,5R)-diastereomer δ 8.39 (br s, 1H), 7.81 (d, 1H), 6.28 (br s, 2H), 3.94 (d, 1H), 3.75 (d, 1H), 1.49 (s, 3H), 1.41 (s, 3H);

(2R,5S)-diastereomer δ 8.37 (d, 1H), 7.68 (d, 1H), 6.34 (br s, 2H), 3.91 (d, 1H), 3.83 (d, 1H), 1.59 (s, 3H), 1.40 (s, 3H).

### l) 5-Cyano-3-methyl-pyridine-2-carboxylic acid [44(3R,6R)-5-amino-3,6-dimethyl-6-trifluoromethyl-3,6-dihydro-2H-[1,4]oxazin-3-yl)-5-fluoro-pyridin-2-yl]-amide

A mixture of 5-cyano-3-methyl-pyridine-2-carboxylic acid amide (43.5 mg, 0.270 mmol), (2R,5R)-5-(2-bromo-5-fluoro-pyridin-4-yl)-2,5-dimethyl-2-trifluoromethyl-5,6-dihydro-2H-[1,4]-oxazin-3-ylamine (100.0 mg, 0.270 mmol), Xantphos (14.1 mg, 0.024 mmol) and Cs₂CO₃ (123.0 mg, 0.378 mmol) in dioxane (2.5 ml) was degassed with argon for 5 min, then Pd₂(dba)₃ (7.42 mg, 8.11 μmol) was added and the reaction mixture was stirred at 60° C. for 24 h. The reaction mixture was diluted with H₂O and TBDME. The phases were separated and the aq. phase was twice reextracted with TBDME. The combined org. phases were washed with brine, dried over Na₂SO₄, filtered and concentrated. Prep HPLC (Alltech Grom Saphir 65 Si 10 μM column, 150×30 mm, gradient n-heptane:EtOAc:MeOH 0-1.2 min 68:30:2, 1.2-9 min 0:80:20, 9-12 min 0:65:35, flow: 50 ml/min, detection: 254 nm) yielded the parent compound as a colourless solid. Rt_{H11}=0.83 min; ESIMS: 451 [(M+H)⁺]; ¹H NMR (400 MHz, DMSO-d₆): δ 10.80 (br s, 1H), 8.98 (br s, 1H), 8.42 (s, 1H), 8.36 (dd, 1H), 8.30 (dd, 1H), 6.24 (br s, 2H), 3.97 (d, 1H), 3.82 (d, 1H), 2.58 (s, 3H), 1.49 (s, 3H), 1.44 (s, 3H).

The compound in Table 30 can be prepared by a procedure analogous to that used in Example 272.

TABLE 30

| | | | MS |
|---|---|---|---|
| | | ¹H-NMR | [m/z; |
| Example | Compound | (δ; DMSO-d₆) | (M + 1)⁺] |
| 273 | 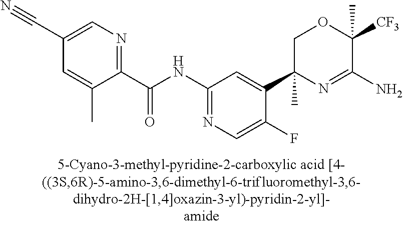 [SMILES: Cc1cc(C#N)cnc1C(=O)Nc1cc([C@@]2(C)CO[C@@](C)(C(F)(F)F)C(N)=N2)c(F)cn1] | 10.75 (s, 1H), 8.97 (s, 1H), 8.42 (s, 2H), 8.27 (d, 1H), 6.21 (br s, 2H), 3.91 (s, 2H), 2.58 (s, 3H), 1.61 (s, 3H), 1.44 (s, 3H). | 451 |

### Example 274

### 5-Bromo-pyridine-2-carboxylic acid [5-(5-amino-3-fluoromethyl-3,6-dihydro-2H-[1,4]oxazin-3-yl)-6-chloro-pyridin-3-yl]-amide hydrochloride

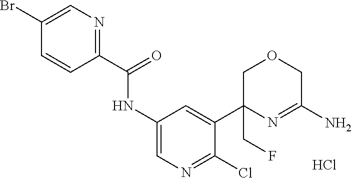 [SMILES: NC1=NC(CF)(c2cc(NC(=O)c3ccc(Br)cn3)cnc2Cl)COC1 ; Cl]

### a) 5-Bromo-2-chloro-3-nitromethyl-pyridine

To a solution of 5-bromo-3-bromomethyl-2-chloro-pyridine (4.10 g, 14.37 mmol) in TBME (50.3 ml) in a tin-foil wrapped flask was added silver nitrite (2.65 g, 17.24 mmol) and the reaction mixture was stirred at room temperature for 15 h. The solid was filtered off, rinsed with TBME and the filtrate was evaporated. The residue was purified by chromatography on silica gel (cyclohexane to cyclohexane/EtOAc 3:2) to provide the title compound as pale brown oil.

HPLC: Rt_{H11}=0.91 min; ESIMS [M−H]⁻=248.9, 251.0; ¹H-NMR (600 MHz, DMSO-d₆): 8.71 (d, 1H), 8.40 (d, 1H), 5.92 (s, 2H).

### b) 2-(5-Bromo-2-chloro-pyridin-3-yl)-2-nitropropane-1,3-diol

To a solution of 5-bromo-2-chloro-3-nitromethyl-pyridine (286 mg, 1.14 mmol) in dioxane (2.3 ml) was added 35% aq. formaldehyde (215 mg, 2.50 mmol), triethylamine (0.079 ml, 0.57 mmol) and the reaction mixture was stirred at room temperature for 2 h, to the mixture was added a mixture of saturated aq. NaCl and 12N HCl (0.05 ml, 0.6 mmol). Then the mixture was extracted with TBME the combined organic layers were washed with saturated aq. NaCl, dried with Na₂SO₄ and evaporated. The residue was purified by chromatography on silica gel (cyclohexane to cyclohexane/EtOAc 1:1) to provide the title compound as colorless solid. M.p. 162-163° C. HPLC: Rt_{H11}=0.69 min; ESIMS [M+H]⁺=311.0, 313.0; ¹H NMR (600 MHz, DMSO-d₆): 8.64 (d, 1H), 8.11 (d, 1H), 5.60 (t, 2H), 4.34 (dd, 2H), 4.19 (dd, 2H).

### c) 2-(5-Bromo-2-chloro-pyridin-3-yl)-2-nitropropane-1,3-diol

To a suspension of zinc dust (2.03 g, 31 mmol) in acetic acid (8.6 ml) was added dropwise within 1 h a solution of 2-(5-bromo-2-chloro-pyridin-3-yl)-2-nitropropane-1,3-diol (1.61 g, 5.17 mmol) in acetic acid (17.3 ml) and DMF (5.2 ml), while maintaining the temperature between 30 and 40° C. (ice cooling), the reaction mixture was stirred at 40° C. for 1.5 h. The mixture was filtered, the residue rinsed with methanol and at 0° C. the filtrate poured on a 1:1 mixture of EtOAc and saturated aq. NaHCO₃. The pH was adjusted to 12 by addition of 1N NaOH, the layers separated and the aq. phase extracted with EtOAc. The combined organic layers were washed with saturated aq. NaCl, dried with Na₂SO₄ and evaporated to provide the title compound as yellow solid. HPLC: Rt_{H12}=0.22 min; ESIMS [M+H]⁺=281.0, 283.0; ¹H NMR (400 MHz, DMSO-d₆): 8.43 (d, 1H), 8.38 (d, 1H), 4.80 (t, 2H), 3.93 (dd, 2H), 3.67 (dd, 2H), 2.18 (br. s, 2H).

### d) N-[1-(5-Bromo-2-chloro-pyridin-3-yl)-2-hydroxy-1-hydroxymethyl-ethyl]-2-chloro-acetamide

To a suspension of 2-(5-bromo-2-chloro-pyridin-3-yl)-2-nitropropane-1,3-diol (904 mg, 3.21 mmol) in DCM (64 ml) was added pyridine (2.6 ml, 32.1 mmol), after cooling to −30° C. a solution of chloro-acetylchloride (1.022 ml, 12.84 mmol) in DCM (32 ml) was added within 10 min., the reaction mixture was stirred at −30° C. for 1.5 h. At −30° C. 1M HCl and DCM was added, the layers were separated, the aq. phase extracted with DCM and the combined organic layers washed with halfsaturated aq. NaHCO₃ and halfsaturated aq. NaCl, dried with Na₂SO₄ and evaporated. The obtained per-acetylated product was dissolved in methanol (19.3 ml) and K₂CO₃ powder (222 mg, 1.6 mmol) added, the mixture was stirred at room temperature for 30 min. After addition of 1M HCl and TBME the layers were separated, the aq. layer was extracted with TBME, the combined organic layers were washed with halfsaturated aq. NaCl, dried with Na₂SO₄ and evaporated. The residue was purified by chromatography on silica gel (cyclohexane/EtOAc 1:0 to cyclohexane/EtOAc 0:1) to provide the title compound as colorless solid.

HPLC: Rt_{H12}=0.51 min; ESIMS [M+H]⁺=356.9, 358.9; ¹H NMR (600 MHz, DMSO-d₆): 8.44 (d, 1H), 8.27 (s, 1H), 7.99 (s, 1H), 5.08 (t, 2H), 4.11 (s, 2H), 4.00-3.95 (m, 2H), 3.94-3.89 (m, 2H).

### e) 5-(5-Bromo-2-chloro-pyridin-3-yl)-5-hydroxymethyl-morpholin-3-one

To a suspension of N-[1-(5-bromo-2-chloro-pyridin-3-yl)-2-hydroxy-1-hydroxymethyl-ethyl]-2-chloro-acetamide (622 mg, 1.74 mmol) in tert.-butanol (10.2 ml) was added at 0° C. potassium tert.-butoxide (292 mg, 2.61 mmol), the reaction mixture was stirred at room temperature for 1 h. Water was added and the tert.-butanol evaporated, the mixture was extracted with EtOAc, the combined organic layers were washed with halfsaturated aq. NaCl, dried with Na₂SO₄ and evaporated to provide the title compound as beige foam.

HPLC: Rt_{H11}=0.58 min; ESIMS [M+H]⁺=320.9, 322.9; ¹H NMR (600 MHz, DMSO-d₆): 8.56 (d, 1H), 8.39 (s, 1H), 8.21 (d, 1H), 5.44 (t, 1H), 4.42 (d, 1H), 4.04 (s, 2H), 3.94 (dd, 1H), 3.90 (d, 1H), 3.86 (d, 1H).

### f) 5-(5-Bromo-2-chloro-pyridin-3-yl)-5-fluoromethyl-morpholin-3-one

To a suspension of 5-(5-bromo-2-chloro-pyridin-3-yl)-5-hydroxymethyl-morpholin-3-one (547 mg, 1.70 mmol) in THF (13.6 ml) was added at 0° C. within 5 min a solution of DAST (1.01 ml, 7.65 mmol) in THF (7.2 ml), the reaction mixture was stirred at room temperature for 6 h. The mixture was cooled to 0° C., halfsaturated aq. Na₂CO₃ was added and the mixture was extracted with EtOAc, the combined organic layers were washed with halfsaturated aq. NaCl, dried with Na₂SO₄ and evaporated. The residue was purified by chromatography on silica gel (cyclohexane to cyclohexane/EtOAc 1:4) to provide the title compound as colorless solid.

HPLC: Rt_{H12}=0.66 min; ESIMS [M−H]⁻=320.8, 322.8; ¹H NMR (600 MHz, DMSO-d₆): 8.80 (s, 1H), 8.63 (d, 1H), 8.12 (d, 1H), 5.01-4.93 (m, 1H), 4.92-4.85 (m, 1H), 4.37 (dd, 1H), 4.10 (s, 2H), 3.95 (d, 1H).

### g) 5-[5-(Benzhydrylidene-amino)-2-chloro-pyridin-3-yl)-5-fluoromethyl-morpholin-3-one

To a solution of 5-(5-bromo-2-chloro-pyridin-3-yl)-5-fluoromethyl-morpholin-3-one (199 mg, 0.615 mmol), benzophenone imine (86 mg, 0.473) and Cs₂CO₃ (620 mg, 1.89 mmol) in toluene (4.6 ml) and dioxane (4.6 ml) was added Pd₂(dba)₃ (22 mg, 0.024 mmol) and Xantphos (41 mg, 0.071 mmol) and the mixture was purged with nitrogen, the reaction mixture was heated to 100° C. for 4 h. After cooling to 0° C. water was added and the mixture was extracted with EtOAc, the combined organic layers were washed with water, dried with Na₂SO₄ and evaporated. The residue was purified by chromatography on silica gel (cyclohexane to cyclohexane/EtOAc 1:4) to provide the title compound as yellowish foam.

HPLC: Rt_{H12}=1.11 min; ESIMS [M+H]⁺=424.1; ¹H NMR (600 MHz, DMSO-d₆): 8.71 (s, 1H), 7.80 (s, 1H), 7.70 (d, 2H), 7.58 (t, 1H), 7.50 (t, 2H), 7.36 (d, 4H), 7.16 (d, 2H), 4.87-4.70 (m, 2H), 4.28 (d, 1H), 4.04 (d, 1H), 3.93 (d, 1H), 3.80 (d, 1H).

### h) 5-(5-Amino-2-chloro-pyridin-3-yl)-5-fluoromethyl-morpholine-3-thione

To a solution of 5-[5-(benzhydrylidene-amino)-2-chloro-pyridin-3-yl)-5-fluoromethyl-morpholin-3-one (206 mg, 0.467 mmol) in THF (2.4 ml) was added Lawessons's reagent (189 mg, 0.467 mmol), the reaction mixture was heated to reflux for 1 h. The solvent was evaporated and the crude product dissolved in THF (12 ml), 2M HCl (6.3 ml) were added and the mixture stirred at room temperature for 17 h. After cooling to 0° C. aq. 2M K₂CO₃ was added and the basic mixture was extracted with EtOAc, the combined organic layers were washed with halfsaturated aq. NaCl, dried with Na₂SO₄ and evaporated. The residue was purified by chromatography on silica gel (cyclohexane/EtOAc 1:0 to cyclohexane/EtOAc 0:1) to provide the title compound as beige foam.

HPLC: Rt_{H12}=0.59 min; ESIMS [M+H]⁺=276.0; ¹H NMR (600 MHz, DMSO-d₆): 10.99 (s, 1H), 7.70 (d, 1H), 7.08 (d, 1H), 5.76 (s, 2H), 4.99 (dd, 1H), 4.82 (dd, 1H), 4.46-4.35 (m, 3H), 3.96 (d, 1H).

### i) 5-Bromo-pyridine-2-carboxylic acid [6-chloro-5-(3-fluoromethyl-5-thioxo-morpholin-3-yl)-pyridin-3-yl]-amide

A solution of 5-(5-amino-2-chloro-pyridin-3-yl)-5-fluoromethyl-morpholine-3-thione (33 mg, 0.12 mmol), 5-bromo-pyridine-2-carboxylic acid (36 mg, 0.18 mmol) and HOAt (29 mg, 0.215 mmol) in DMF (0.4 ml) was cooled to 0° C. and DIPEA (0.042 ml, 0.24 mmol) and EDC (34 mg, 0.18 mmol) were added, the reaction mixture was stirred at 0° C. for 10 min, then allowed to warm to room temperature over night. At 0° C. aq. 1M KHCO₃ was added and the mixture extracted with toluene. The combined organic layers were washed with water, dried with Na₂SO₄ and evaporated. The residue was taken up in DCM/MeOH 65/35 from which the product started to crystallize. Filtration, rinsing of the crystallized material with DCM and drying provide the title compound as yellow crystals. TLC (cyclohexane/EtOAc 1:1) R_{f}=0.45; HPLC: Rt_{H12}=1.08 min; ESIMS [M+H]⁺=458.9, 461.0.

### j) 5-Bromo-pyridine-2-carboxylic acid [5-(5-amino-3-fluoromethyl-3,6-dihydro-2H-[1,4]oxazin-3-yl)-6-chloro-pyridin-3-yl]-amide hydrochloride

To a suspension of 5-bromo-pyridine-2-carboxylic acid [6-chloro-5-(3-fluoromethyl-5-thioxo-morpholin-3-yl)-pyridin-3-yl]-amide (26 mg, 0.057 mmol) in 7M NH₃ in MeOH (0.23 ml) was added at −20° C., tert.-butylhydroperoxide (0.055 ml, 0.566 mmol) and aq. 25% NH₃ (0.15 ml, 0.99 mmol), the reaction mixture was stirred at room temperature for 80 min, 7M NH₃ in MeOH (0.69 ml) were added and stirring continued for 20 h. At 0° C. halfsaturated aq. Na₂S₂O₃ was added and the mixture extracted with EtOAc. The combined organic layers were washed with halfsaturated aq. NaCl, dried with Na₂SO₄ and evaporated. The residue was purified by preparative TLC DCM/MeOH 9:1 to yield the desired compound as colorless foam. The product was dissolved in DCM/MeOH, 5 equivalents of 5M HCl in Et₂O were added and the solvents evaporated to provide the title compound as beige solid.

TLC (DCM/MeOH 9:1) R_{f}=0.22; HPLC: Rt_{H12}=0.71 min; ESIMS [M+H]⁺=442.0, 443.9; ¹H NMR (600 MHz, DMSO-d₆): 11.12 (s, 1H), 8.88 (d, 1H), 8.86 (s, 1H), 8.65 (d, 1H), 8.35 (dd, 1H), 8.09 (d, 1H), 6.02 (br. s, 2H), 4.80-4.66 (m, 2H), 4.13-3.93 (m, 4H).

### Preparation of Intermediates

The substituted acid building blocks were either commercially available or can be prepared as described in the literature or in an analogous manner, e.g. WO 2005063738, WO 2009091016, WO 2010047372, Bioorg. Med. Chem. 2001, 9, 2061-2071, or can be prepared as described hereafter or in an analogous manner.

### Acid-1: 5-Cyano-4,6-dideutero-3-trideuteromethyl-pyridine-2-carboxylic acid

### a) 5-Bromo-4,6-dideutero-3-trideuteromethyl-pyridine-2-carboxylic acid

A suspension of 2.16 g (0.00 mmol) 5-bromo-3-methyl-pyridine-2-carboxylic acid in 36 ml of D₂O (99.96% D) was treated with 4 ml of a 40% solution of NaOD in D₂O. The homogeneous solution was heated in a 100 ml Teflon vessel with a Synthos 3000 Microwave apparatus. The mixture was heated at 160° C. for 5 h and cooled down. 1H-NMR and MS analises of the product showed that deuteration had progressed to a high degree. Only minor amounts of tetradeutero derivatives were present. The reaction mixture was acidified to pH3 with 2N HCl and extracted with EtOAc. The organic phase was dried with MgSO4.H₂O and evaporated to give the title compound as a white solid, pure enough for further transformations.

HPLC: Rt_{H2}=2.829 min; ESIMS [M+H]⁺=221, 223 (1Br, 5D);

### b) 5-Bromo-4,6-dideutero-3-trideuteromethyl-pyridine-2-carboxylic acid tert-butyl ester

A solution of 1.65 g (7.46 mmol) 5-bromo-4,6-dideutero-3-trideuteromethyl-pyridine-2-carboxylic acid and two drops of DMF were dissolved in 17 ml DCM. Oxalyl chloride (1.3 ml, 14.9 mmol) was added dropwise. The development of gas started immediately. After stirring for 2 h at 25° C. the mixture was evaporated, taken up in toluene and evaporated again. The residual brownish resin was dissolved in 3 ml THF and added to a stirred solution of 14 ml (22.39 mmol) BuLi (1.6 M in hexane) in 24 ml t-BuOH. After 1 h the mixture was poured onto 10% aqueous NH4Cl and extracted with TBME. The organic layer was washed with brine, dried with MgSO4.H2O and evaporated. Chromatography on silica gel (hexane/EtOAc 9:1) provided the title compound as a colorless liquid.

HPLC: Rt_{H1}=3.002 min; ESIMS [M+H]⁺=277, 279 (1Br, 5D);

¹H-NMR (360 MHz, CDCl₃): 1.65 (s, 9H).

### c) 5-Cyano-4,6-dideutero-3-trideuteromethyl-pyridine-2-carboxylic acid tert-butyl ester

A mixture of 1.41 g (5.09 mmol) 5-bromo-4,6-dideutero-3-trideuteromethyl-pyridine-2-carboxylic acid tert-butyl ester, 0.418 g (3.56 mmol) Zn(CN)₂, 0.033 g Zn powder (0.509 mmol) and 0.265 g (0.254 mmol) Pd2(dba)₃.CHCl3 were suspended in 14 ml DMF under nitrogen atmosfere. A 0.25 M solution of tBu3P in dioxane (4.0 ml, 1.02 mmol) was added and the mixture was stirred for 16 h at 60° C. After being cooled down the mixture was diluted with TBME, filtered over celite and washed with brine three times. The crude product was purified by column chromatography on silica gel (hexane/EtOAc 5-15%) to give the title compound as an off white solid.

HPLC: Rt_{H3}=3.275 min; ESIMS [M+Na]⁺=246 (5D);

¹H-NMR (360 MHz, CDCl₃): 1.68 (s, 9H);

Ft-IR: 2231 cm⁻¹ (CN).

### d) 5-Cyano-4,6-dideutero-3-trideuteromethyl-pyridine-2-carboxylic acid

To a solution of 825 mg (3.69 mmol) 5-cyano-4,6-dideutero-3-trideuteromethyl-pyridine-2-carboxylic acid tert-butyl ester in 5.1 g (37 mmol) 1,3-dimethoxybenzene were added 8.3 ml TFA and stirred for 6.5 h. The reaction mixture was diluted with toluene and evaporated. The residue was taken up in toluene and evaporated (2×). The product was crystallized from TBME/hexane to give the title compound as a white powder.

HPLC: Rt_{H2}=2.397 min; ESIMS [M+H]⁺=168 (5D);

¹H-NMR (360 MHz, CDCl₃): non-deuterated impurities.

### Acid-2: 5-Chloro-4,6-dideutero-3-trideuteromethyl-pyridine-2-carboxylic acid

The title compound was prepared by an analogous procedure as Acid-1 steps a) to b).

HPLC: Rt_{H2}=2.820 min; ESIMS [M+H]⁺=177 (5D);

¹H-NMR (360 MHz, D₂O): non deuterated impurities.

### Acid-3: 5-Cyano-3-methyl-pyridine-2-carboxylic acid

The title compound was prepared by an analogous procedure to Acid-1 starting with 5-bromo-3-methyl-pyridine-2-carboxylic acid instead of the deuterated derivative [Acid-1 step a)].

Rf (hexanes/EtOAc 6:1)=0.28

¹H-NMR (360 MHz, CDCl₃): 8.09 (dd, 1H), 7.79 (ddd, 1H), 7.17 (t, 1H), 6.44 (t, J=45 Hz, 1H).

### Acid-4: 3,5-Dimethoxy-pyridine-2-carboxylic acid

A suspension of 3,5-dimethoxy-pyridine-2-carbonitrile (CAS: 36057-45-1, 2.71 g, 16.51 mmol) in 45 ml MeOH and 65 ml 30% aq NaOH was refluxed for 6 h. MeOH was removed by evaporation and the residue was washed with TBME. The aq phase was acidified with conc. HCl till the pH was 3. The mixture was extracted with EtOAc and THF. The combined org layers were dried with sodium sulfate and evaporated. The brown solid was crystallized from EtOH to provide the title compound as pale brown crystals.

HPLC: Rt_{H2}=2.183 min; ESIMS [M+H]⁺=184;

¹H-NMR (360 MHz, DMSO-d6): 12.61 (s, 1H), 3.93 (s, 3H), 3.88 (s, 3H).

### Acid-5: 5-Difluoromethoxy-3-methyl-pyridine-2-carboxylic acid

### a) 5-Difluoromethoxy-3-methyl-pyridine-2-carbonitrile

A solution of 5-hydroxy-3-methyl-pyridine-2-carbonitrile (CAS registry 228867-86-5) (228 mg, 1.70 mmol), sodium chlorodifluoroacetate (CAS registry 1895-39-2) (518 mg, 3.40 mmol) and K₂CO₃ (705 mg, 5.10 mmol) in DMF (7 ml) was stirred for 0.5 h at 100° C. The reaction mixture was diluted with EtOAc and washed with saturated aqueous NH₄Cl soln. and brine. The aqueous layers were reextracted with EtOAc, the combined organic layers dried over Na₂SO₄, filtrated and the filtrate was concentrated. The title compound was obtained as a colourless oil after flash chromatography on silica gel (cyclohexane/EtOAc gradient 0-3 min 95:5, 3-35 min 95:5 to 60:40).

HPLC Rt_{H10}=0.87 min; ESIMS: 185 [(M+H)⁺];

¹H NMR (400 MHz, CDCl₃): 8.40 (d, 1H), 7.45 (d, 1H), 6.64 (t, 1H), 2.61 (s, 3H).

### b) 5-Difluoromethoxy-3-methyl-pyridine-2-carboxylic acid

To a solution of 5-difluoromethoxy-3-methyl-pyridine-2-carbonitrile (145 mg, 0.787 mmol) in EtOH (5 ml) was added 1M aqueous NaOH soln. (2.5 ml). The reaction mixture was stirred for 7 h at 70° C., then for 9 h at room temperature. It was diluted with Et₂O and twice extracted with water. The combined aqueous layers were reextracted with Et₂O, acidified to pH 2 with 1M aqueous HCl and twice extracted with TBME. The combined organic layers were dried over Na₂SO₄, filtrated and the filtrate was concentrated to yield the title compound as a white solid which was used for the next step without further purification.

HPLC Rt_{H10}=0.61 min; ESIMS: 204 [(M+H)⁺];

¹H NMR (400 MHz, MeOD): 8.32 (d, 1H), 7.61 (d, 1H), 7.06 (t, 1H), 2.64 (s, 3H).

### Acid-6: 5-Fluoromethoxy-3-methyl-pyridine-2-carboxylic acid

### a) 5-Fluoromethoxy-3-methyl-pyridine-2-carbonitrile

To a solution of 5-hydroxy-3-methyl-pyridine-2-carbonitrile (CAS registry 228867-86-5) (228 mg, 1.70 mmol) in DMF (10 ml) was added a solution of toluene-4-sulfonic acid fluoromethyl ester (CAS registry 114435-86-8) (521 mg, 2.55 mmol) and Cs₂CO₃ (1.386 g, 4.26 mmol) in DMF (4 ml). The reaction mixture was stirred for 1 h at 100° C., then for 1 h at 70° C., diluted with EtOAc and washed with saturated aqueous NH₄Cl soln. and brine. The aqueous layers were reextracted with EtOAc, the combined organic layers dried over Na₂SO₄, filtrated and the filtrate was concentrated. The title compound was obtained as a white solid after flash chromatography on silica gel (cyclohexane/EtOAc gradient 0-3 min 95:5, 3-30 min 95:5 to 65:35).

HPLC Rt_{H10}=0.77 min; ESIMS: 167 [(M+H)⁺];

¹H NMR (400 MHz, CDCl₃): 8.36 (d, 1H), 7.34 (d, 1H), 5.79 (d, 2H), 2.59 (s, 3H).

### b) 5-Fluoromethoxy-3-methyl-pyridine-2-carboxylic acid

To a solution of 5-fluoromethoxy-3-methyl-pyridine-2-carbonitrile (118 mg, 0.71 mmol) in EtOH (4 ml) was added 1M aqueous NaOH soln. (2 ml). The reaction mixture was stirred for 7 h at 70° C., then for 9 h at room temperature. It was diluted with TBME and twice washed with water. The combined aqueous layers were reextracted with TBME, acidified to pH 2 with 1M aqueous HCl and twice extracted with TBME. The combined organic layers were dried over Na₂SO₄, filtrated and the filtrate was concentrated to yield the title compound as a white solid which was used for the next step without further purification.

HPLC Rt_{H10}=0.50 min; ESIMS: 186 [(M+H)⁺]

¹H NMR (400 MHz, MeOD): 8.28 (d, 1H), 7.55 (d, 1H), 5.88 (d, 2H), 2.66 (s, 3H).

### Acid-7: 5-(2-Methoxy-ethoxy)-pyridine-2-carboxylic acid

### a) 5-(2-Methoxy-ethoxy)-pyridine-2-carboxylic acid methyl ester

To a precooled solution of 5-hydroxy-pyridine-2-carboxylic acid methyl ester (CAS registry 30766-12-2) (150 mg, 0.980 mmol) and 2-methoxyethanol (82 mg, 0.085 ml, 1.077 mmol) in THF (10 ml) was added at 0° C. triphenylphosphine (397 mg, 1.469 mmol) and the reaction mixture was stirred for 10 min at 0° C. A solution of DIAD (316 mg, 1.469 mmol) in THF (5 ml) was added and the mixture was stirred at room temperature for 19.5 h. After dilution with EtOAc, the crude mixture was extracted with water and brine, the aqueous layers were reextracted with EtOAc, the combined organic extracts dried over Na₂SO₄, filtered and the filtrate concentrated to yield the title compound after flash chromatography on silica gel (DCM/EtOAc gradient 0-3 min 60:40; 3-35 min 60:40 to 25:75).

HPLC Rt_{H10}=0.63 min; ESIMS: 212 [(M+H)⁺];

¹H NMR (400 MHz, CDCl₃): 8.47 (d, 1H), 8.14 (d, 1H), 7.35 (dd, 1H), 4.27-4.24 (m, 2H), 4.00 (s, 3H), 3.82-3.79 (m, 2H), 3.47 (s, 3H).

### b) 5-(2-Methoxy-ethoxy)-pyridine-2-carboxylic acid

To a solution of 5-(2-methoxy-ethoxy)-pyridine-2-carboxylic acid methyl ester (390 mg, 0.489 mmol) in THF (3 ml) was added 1M aqueous NaOH (0.538 ml). The reaction mixture was stirred at room temperature for 2.5 h, concentrated, the residue was dissolved in EtOAc and washed twice with water. The aqueous layers were acidified with 1M aqueous HCl (0.538 ml) and the title compound was isolated by lyophilisation.

HPLC Rt_{H10}=0.42 min; ESIMS: 198 [(M+H)⁺];

¹H NMR (400 MHz, DMSO-d₆): 8.37 (d, 1H), 8.01 (d, 1H), 7.52 (dd, 1H), 4.28-4.24 (m, 2H), 3.71-3.67 (m, 2H), 3.31 (s, 3H).

### Acid-8: 3-Fluoro-5-(2-methoxy-ethoxy)-pyridine-2-carboxylic acid

### a) 2-Chloro-3-fluoro-5-(2-methoxy-ethoxy)-pyridine

To a solution of 6-chloro-5-fluoro-pyridin-3-ol (CAS registry 870062-76-3) (800 mg, 5.42 mmol), 2-methoxy-ethanol (454 mg, 0.471 ml, 5.96 mmol) and triphenylphosphine (2.199 g, 8.13 mmol) in THF (40 ml) was added dropwise a solution of DIAD (1.731 g, 8.13 mmol) in THF (20 ml) while keeping the temperature at 0-5° C. The reaction mixture was stirred for 20 h at room temperature, water and brine were added and the mixture was diluted with EtOAc. The aqueous layer was twice extracted with EtOAc, the combined organic layers were dried over Na₂SO₄, filtered, the filtrate was concentrated and yielded after trituration with Et₂O and filtration, the title compound as a white solid. The filtrate yielded another batch of the product after purification by prep. NP HPLC using an Alltech Grom Saphir 65 Si 10 μM 250×50 mm column (heptane/EtOAc, gradient 0-1.7 min 15% EtOAc, 1.7-17 min 15-100% EtOAc, 17-24.3 min 100% EtOAc, 24.3-27.8 min 0% EtOAc).

HPLC Rt_{H10}=0.86 min; ESIMS: 206 [(M+H)⁺];

¹H NMR (400 MHz, CDCl₃): 7.97 (d, 1H), 7.12 (dd, 1H), 4.25-4.08 (m, 2H), 3.83-3.68 (m, 2H), 3.46 (s, 3H).

### b) 3-Fluoro-5-(2-methoxy-ethoxy)-pyridine-2-carbonitrile

To a solution of 2-chloro-3-fluoro-5-(2-methoxy-ethoxy)-pyridine (806 mg, 3.92 mmol) and Zn(CN)₂ (486 mg, 4.12 mmol) was added under a N₂ atmosphere Pd(PPh₃)₄ (362 mg, 0.314 mmol). The reaction mixture was stirred for 20 min at 120° C. in a microwave, diluted with water and TBME. The insolubles were filtered, the phases were separated and the aqueous layer was extracted twice with TBME. The combined organic layers were washed with brine, dried over Na₂SO₄, filtered and the solvent was removed to leave the title compound as a pale brown oil that was purified by prep. NP HPLC using an Alltech Grom Saphir 65 Si 10 μM 250×50 mm column (heptane/EtOAc, gradient 0-1.7 min 25% EtOAc, 1.7-17 min 25-100% EtOAc, 17-24.3 min 100% EtOAc, 24.3-27.8 min 0% EtOAc).

HPLC Rt_{H10}=0.78 min; ESIMS: 197 [(M+H)⁺];

¹H NMR (400 MHz, CDCl₃): 8.27 (dd, 1H), 7.10 (dd, 1H), 4.34-4.17 (m, 2H), 3.87-3.71 (m, 2H), 3.45 (s, 3H).

### c) 3-Fluoro-5-(2-methoxy-ethoxy)-pyridine-2-carboxylic acid

A solution of 3-fluoro-5-(2-methoxy-ethoxy)-pyridine-2-carbonitrile in 5 ml aqueous 2M NaOH was stirred at 120° C. for 20 min in a microwave. The reaction mixture was diluted with H₂O and the pH was adjusted to 1-1.5. The mixture was extracted with DCM three times and the combined organic phases were dried over Na₂SO₄, filtered and the solvent was removed to yield the crude title compound that was purified by prep. RP HPLC using a Waters SunFire C18 OBD 5 μM 19×150 mm column (A/B: water/ACN+0.1% TFA, gradient 0-1 min 5% B, 1-7 min 5 to 90% B, 7-7.5 min 90% B, 7.5-8 min 90 to 5% B, 8-10 min 5% B) to yield its TFA salt.

HPLC Rt_{H10}=0.50 min; ESIMS: 216 [(M+H)⁺];

¹H NMR (400 MHz, CDCl₃): 8.22 (br s, 1H), 7.15 (dd, 1H), 6.29 (br s, 2H), 4.38-4.19 (m, 2H), 3.89-3.74 (m, 2H), 3.47 (s, 3H).

The TFA salt was converted to the corresponding HCl salt by trituration with HCl/dioxane and subsequent evaporation.

### Acid-9: 5-Methoxy-3-methyl-pyrazine-2-carboxylic acid

### a) 3-Methyl-4-oxy-pyrazine-2-carboxylic acid methyl ester

To a solution of 2.0 g (13.14 mmol) 3-methyl-pyrazine-2-carboxylic acid methyl ester in 40 ml CHCl₃ was added 3.24 g (13.14 mmol) meta-chlorperoxybenzoic acid and the resulting mixture was heated to reflux for 1.5 h. The reaction mixture was basified with saturated aqueous NaHCO₃ and extracted with CHCl₃, the combined organic layers were dried with Na₂SO₄ and evaporated. The residue was purified by chromatography on silica gel (DCM to DCM/MeOH 9:1) to provide the title compound as colorless solid.

HPLC: Rt_{H11}=0.40 min; ESIMS [M+H]⁺=169;

¹H NMR (600 MHz, DMSO-d₆): 8.56 (d, 1H), 8.48 (d, 1H), 3.33 (s, 3H).

### b) 5-Chloro-3-methyl-pyrazine-2-carboxylic acid methyl ester

To a solution of 575 mg (3.4 mmol) 3-methyl-4-oxy-pyrazine-2-carboxylic acid methyl ester in 6.8 ml DMF was added 1.141 ml (1.88 g, 12.24 mmol) phosphoryl trichloride and the resulting mixture was heated to 120° C. for 15 min. After cooling to room temperature ice was added and the mixture was extracted with toluene. The combined organic layers were washed with halfsaturated aqueous NaCl, dried with Na₂SO₄ and evaporated to provide the title compound as brownish solid in a 3:2 mixture with the undesired 6-chloro-3-methyl-pyrazine-2-carboxylic acid methyl ester. The mixture was used in the next step without further purification.

HPLC: Rt_{H10}=0.70 min; ESIMS [M+H]⁺=187.1;

¹H NMR (600 MHz, DMSO-d₆, 5-Cl isomer): 8.74 (s, 1H), 3.90 (s, 3H), 2.71 (s, 3H).

### c) 5-Methoxy-3-methyl-pyrazine-2-carboxylic acid methyl ester

At 0° C. 58 mg (1.458 mmol) 60% sodium hydride in oil was added in portions to 7.3 ml MeOH and the mixture was stirred at room temperature for 30 min. After re-cooling to 0° C. 272 mg (1.458 mmol) of the crude product of the previous step was added as a suspension in 1.7 ml MeOH and the mixture was heated to 50° C. for 1 h. At 0° C. halfsaturated aqueous NH₄Cl was added and the mixture was extracted with EtOAc. The combined organic layers were washed with halfsaturated aqueous NaCl, dried with Na₂SO₄ and evaporated. The residue was purified by chromatography on silica gel (cyclohexane to cyclohexane/EtOAc 4:1) to provide the title compound as brownish solid.

HPLC: Rt_{H10}=0.69 min; ESIMS [M+H]⁺=183.1;

¹H NMR (600 MHz, DMSO-d₆): 8.21 (s, 1H), 3.97 (s, 3H), 3.84 (s, 3H), 2.67 (s, 3H).

### d) 5-Methoxy-3-methyl-pyrazine-2-carboxylic acid

A solution of 105 mg (0.577 mmol) 5-methoxy-3-methyl-pyrazine-2-carboxylic acid methyl ester in 2.6 ml THF was cooled to 0° C., 0.635 ml (0.635 mmol) 1N sodium hydroxide was added dropwise and the mixture was stirred at room temperature for 1.5 h.

After re-cooling to 0° C. 0.635 ml (0.635 mmol) 1N HCl and 1.2 ml toluene were added and the solvents were evaporated to provide the title compound together with sodium chloride as brownish solid. The mixture was used for coupling reactions without further purification.

HPLC: Rt_{H10}=0.50 min; ESIMS [M+H]⁺=169.1;

¹H NMR (600 MHz, DMSO-d₆): 13.04 (br s, 1H), 8.19 (s, 1H), 3.96 (s, 3H), 2.67 (s, 3H).

### Acid-10: 5-(2-Methoxy-ethoxy)-3-methyl-pyrazine-2-carboxylic acid

The title compound was prepared by an analogous procedure to Acid-9 using 2-methoxy-ethanol instead of methanol [Acid-9 step c)].

HPLC: Rt_{H10}=0.54 min; ESIMS [M+H]⁺=213.1;

¹H NMR (600 MHz, DMSO-d₆): 13.04 (br. s., 1H), 8.20 (s, 1H), 4.54-4.40 (m, 2H), 3.80-3.61 (m, 2H), 3.30 (s, 3H), 2.66 (s, 3H).

### Acid-11: 5-But-2-ynyloxy-3-methyl-pyrazine-2-carboxylic acid

The title compound was prepared by an analogous procedure to Acid-9 using but-2-yn-1-01 instead of methanol [Acid-9 step c)].

HPLC: Rt_{H10}=0.78 min; ESIMS [M+H]⁺=207.0;

¹H NMR (360 MHz, DMSO-d₆): 8.23 (s, 1H), 5.06 (d, 2H), 2.68 (s, 3H), 1.87 (t, 3H).

### Acid-12: 3-Amino-5-methoxy-pyrazine-2-carboxylic acid

### a) 3-Amino-5-methoxy-pyrazine-2-carboxylic acid methyl ester

At 0° C. 75 mg (1.866 mmol) 60% sodium hydride in oil was added in portions to 5 ml MeOH and the mixture was stirred at room temperature for 30 min. After re-cooling to 0° C. 350 mg (1.866 mmol) 3-amino-5-chloro-pyrazine-2-carboxylic acid methyl ester (GB 1248146) was added and the mixture was allowed to warm to room temperature and stirred over night.

Saturated aqueous NH₄Cl was added and the mixture was extracted with DCM and EtOAc, the combined organic layers were washed with saturated aqueous sodium chloride, dried with Na₂SO₄ and evaporated. The residue was purified by chromatography on silica gel (cyclohexane to EtOAc) to provide the title compound as colorless solid.

HPLC: Rt_{H10}=0.61 min; ESIMS [M+H]⁺=184.2;

¹H-NMR (360 MHz, DMSO-d₆): 7.52 (s, 1H), 7.49 (br s, 2H), 3.91 (s, 3H), 3.81 (s, 3H).

### b) 3-Amino-5-methoxy-pyrazine-2-carboxylic acid

To a solution of 200 mg (1.092 mmol) 3-amino-5-methoxy-pyrazine-2-carboxylic acid methyl ester in 4 ml THF was added 1.20 ml (1.20 mmol) 1N sodium hydroxide and the mixture was stirred at room temperature for 29 h. To the mixture were added 1.09 ml (1.09 mmol) 1N HCl after stirring for 5 min toluene was added and the solvents were evaporated to provide the title compound together with sodium chloride as colorless solid. The mixture was used for coupling reactions without further purification.

HPLC: Rt_{H11}=0.52 min; ESIMS [M+H]⁺=170.0;

¹H NMR (600 MHz, DMSO-d₆): 12.48 (br s, 1H), 7.57 (br s, 2H), 7.48 (s, 1H), 3.88 (s, 3H).

### Acid-13: 5-tert-Butoxycarbonylamino-2-methyl-oxazole-4-carboxylic acid

### a) 5-tert-Butoxycarbonylamino-2-methyl-oxazole-4-carboxylic acid ethyl ester

To a solution of 221 mg (1.3 mmol) 5-amino-2-methyl-oxazole-4-carboxylic acid ethyl ester in 6.5 ml acetonitrile was added at 0° C. 0.795 ml (4.55 mmol) DIPEA, 31.8 mg (0.26 mmol) DMAP and 709 mg (3.25 mmol) Boc₂O and the mixture was stirred at 45° C. for 2 days. After cooling to room temperature water was added and the mixture was extracted with DCM. The combined organic layers were washed with 1N HCl and halfsaturated aqueous NaCl, dried with Na₂SO₄ and evaporated. The residue was purified by chromatography on silica gel (cyclohexane to cyclohexane/EtOAc 1:1) to provide the title compound as pinkish solid.

HPLC: Rt_{H10}=1.21 min; ESIMS [M+H]⁺=271.1.

### b) 5-tert-Butoxycarbonylamino-2-methyl-oxazole-4-carboxylic acid

To a solution of 314 mg (1.163 mmol) 5-tert-butoxycarbonylamino-2-methyl-oxazole-4-carboxylic acid ethyl ester in 1.16 ml THF was added at 0° C. 5.82 ml (5.82 mmol) 1N sodium hydroxide, the mixture was allowed to warm to room temperature and stirring was continued for 6 days. At 0° C. 5.82 ml (5.82 mmol) 1N HCl was added and the solvents were evaporated. The residue was suspended in DCM and filtered, the solvent was evaporated to provide the title compound as colorless solid.

HPLC: Rt_{H10}=0.62 min; ESIMS [M−H]⁻=241.0;

¹H NMR (600 MHz, DMSO-d₆): 12.80 (br s, 1H), 9.56 (br s, 1H), 2.35 (s, 3H), 1.42 (s, 9H).

### Acid-14: 3-Chloro-5-fluoromethoxy-pyridine-2-carboxylic acid

### a) 3-Chloro-5-hydroxy-pyridine-2-carbonitrile

To a solution of acetic acid 5,6-dichloro-pyridin-3-yl ester (CA 110861-18-2, Synthesis, 1990, 499) (4.88 g, 23.6 mmol) in anhydrous DMF (45 ml) was added after degasing with argon Zn-dust (70 mg, 1.07 mmol), Zn(CN)₂ (1.28 g, 10.9 mmol) and DPPF PdCl₂ (966 mg, 1.18 mmol) and the reaction mixture was heated for 6 h at 130° C. and 18 h at 150° C. The reaction mixture was diluted with TBME and H₂O, filtered over Celite, and the product was extracted with TBME. Combined extracts were washed with water and brine, dried over MgSO₄, filtered and concentrated. The title compound was obtained as a beige solid after crystallization from EtOAc-hexane: TLC (CH₂Cl₂-MeOH 19:1): Rf=0.22;

HPLC Rt_{H5}=0.677 min; ESIMS: 153 and 155 [(M−H)⁻];

¹H NMR (360 MHz, CD₃OD): 8.19 (d, 1H), 7.41 (d, 1H).

### b) 3-Chloro-5-fluoromethoxy-pyridine-2-carbonitrile

To a solution of 3-chloro-5-hydroxy-pyridine-2-carbonitrile (315 mg, 2.03 mmol) in DMF (16 ml) was added Cs₂CO₃ (1.652 g, 5.07 mmol) and toluene-4-sulfonic acid fluoromethyl ester (CAS registry 114435-86-8) (621 mg, 3.04 mmol) and the reaction mixture was heated at 80° C. for 24 h. The solvent was removed under reduced pressure and the residue taken up in TBME, washed with water and brine, dried over MgSO₄, filtered and concentrated. The title compound was obtained as a yellow oil after chromatography on silica gel (hexane-EtOAc 10:1 to 2:1) to provide the title compound as a light yellow oil: TLC (hexane-EtOAc 1:1): Rf=0.62; HPLC Rt_{H5}=0.872 min; ESIMS: 185 and 187 [(M−H)⁻];

¹H NMR (360 MHz, CDCl₃): 8.35 (s, 1H), 7.47 (s, 1H), 5.72 (d, 2H).

### c) 3-Chloro-5-fluoromethoxy-pyridine-2-carboxylic acid

To a solution of 3-chloro-5-fluoromethoxy-pyridine-2-carbonitrile (76 mg, 0.4 mmol) in dioxane (3 ml) was added 1N NaOH (1.4 ml) and the reaction mixture was heated for 30 h at 70° C. The reaction mixture was acidified with 4N HCl to pH 3 and evaporated to dryness. The residue was suspended in CH₂Cl₂-MeOH 8:1, filtered over Celite and concentrated to provide the title compound as a yellow oil.

HPLC Rt_{H5}=0.549 min; ESIMS: 204 and 206 [(M−H)⁻];

¹H NMR (360 MHz, CD₃OD): 8.39 (s, 1H), 7.76 (s, 1H), 5.90 (d, 2H).

### Acid-15: 3-Chloro-5-difluoromethoxy-pyridine-2-carboxylic acid

### a) 3-Chloro-5-difluoromethoxy-pyridine-2-carbonitrile

To a solution of 3-chloro-5-hydroxy-pyridine-2-carbonitrile (314 mg, 2.03 mmol) in DMF (10 ml) was added K₂CO₃ (841 mg, 6.09 mmol) and sodium chlorodifluoroacetate (1.29 g, 8.11 mmol) and the reaction mixture was heated at 100° C. for 10 min. The reaction mixture was diluted with H₂O and extracted with TBME. Combined extracts were washed with brine, dried over MgSO₄, filtered and concentrated. The title compound was obtained as a yellow oil after chromatography on silica gel (hexane-EtOAc 20:1 to 1:1) to provide the title compound as a light yellow oil: TLC (hexane-EtOAc 2:1): Rf=0.54;

HPLC Rt_{H5}=0.968 min; ESIMS: 203 and 205 [(M−H)⁻];

¹H NMR (360 MHz, CDCl₃): 8.41 (d, 1H), 7.59 (d, 1H), 6.61 (t, 1H).

### b) 3-Chloro-5-difluoromethoxy-pyridine-2-carboxylic acid

To a solution of 3-chloro-5-hydroxy-pyridine-2-carbonitrile (90 mg, 0.44 mmol) in dioxane (2 ml) was added 1N NaOH (1.5 ml) and the reaction mixture was heated for 14 h at 70° C. The reaction mixture was extracted with EtOAc, the aqueous layer acidified to pH 3 with 4N HCl and evaporated to dryness. The residue was suspended in CH₂Cl₂-MeOH 10:1, filtered over Celite and concentrated to provide the title compound as beige solid.

HPLC Rt_{H5}=0.667 min; ESIMS: 222 and 224 [(M−H)⁻];

¹H NMR (360 MHz, CD₃OD): 8.46 (s, 1H), 7.87 (s, 1H), 7.12 (t, 1H).

### Acid-16: 3-Chloro-5-methoxymethyl-pyridine-2-carboxylic acid

### a) 3-Chloro-5-methoxymethyl-pyridine-2-carbonitrile

To a solution of 2,3-dichloro-5-methoxymethyl-pyridine (CA registry 202395-72-0) (7.5 g, 38 mmol) in DMF (100 ml) was added after degasing with argon Zn-dust (126 mg, 1.91 mmol), Zn(CN)₂ (2.27 g, 19.1 mmol) and DPPF PdCl₂ (0.997 g, 1.15 mmol) and the reaction mixture was heated for 2 h at 145° C. The reaction mixture was concentrated, the residue redissolved in TBME and 5% aqueous NaHCO₃ solution and extracted with TBME. Combined extracts were washed with water and brine, dried over MgSO₄, filtered and concentrated. The title compound was obtained after chromatography on silica gel (hexane-EtOAc 20:1 to EtOAc) as beige crystals: TLC (hexane-EtOAc 1:1): Rf=0.47;

HPLC Rt_{H5}=0.854 min; ESIMS: 183 and 185 [(M+H)⁺];

¹H NMR (360 MHz, CDCl₃): 8.46 (s, 1H), 7.80 (s, 1H), 4.49 (s, 2H), 3.41 (s, 3H).

### b) 3-Chloro-5-methoxymethyl-pyridine-2-carboxylic acid

To a solution of 3-chloro-5-methoxymethyl-pyridine-2-carbonitrile (2.75 g, 15 mmol) in dioxane (30 ml) was added 2N NaOH (30 ml) and the reaction mixture was heated for 8 h at 75° C. The reaction mixture was acidified to pH 3 with 4N HCl and evaporated to dryness. The residue was suspended in EtOH-THF 1:1, filtered and concentrated. The title compound was obtained after recrystallization from EtOH-TBME as beige crystals.

HPLC Rt_{H5}=0.480 min; ESIMS: 169 and 170 [(M-CH₃OH)⁺];

¹H NMR (360 MHz, CD₃OD): 8.37 (s, 1H), 7.81 (s, 1H), 4.48 (s, 2H), 3.39 (s, 3H).

### Acid-17: 5-Chloro-3-methoxymethyl-pyridine-2-carboxylic acid

### a) (2,5-Dichloro-pyridin-3-yl)-methanol

A 100 ml round bottomed flask was charged with 2,5-dichloropyridine-3-carbaldehyde (Matrix Sci., 3.4 g, 19.32 mmol) followed by addition of ethanol (50 ml). Sodium borohydride was added at room temperature in small portions. After 1 h the starting material was consumed and the reaction was quenched carefully with addition of diluted aq. acetic acid. The reaction mixture was diluted with ethyl acetate, washed with saturated bicarbonate solution and brine, dried over sodium sulfate, filtered and evaporated, to provide the title compound as white solid.

TLC: Rf=0.43 (2:1 cyclohexane:ethyl acetate);

¹H-NMR (400 MHz, CDCl₃): δ 8.29 (d, 1H), 7.94 (d, 1H), 4.80 (d, 2H), 2.23 (broad unresolved triplett, 1H, OH).

### b) 2,5-Dichloro-3-methoxymethyl-pyridine

To a solution of (2,5-dichloro-pyridin-3-yl)-methanol (1000 mg, 5.62 mmol) in dry DMF (25 ml) was added sodium hydride (245 mg, 5.62 mmol, 55% in oil) at 0° C. After 15 minutes methyliodide (0.457 ml, 7.30 mmol) was added and stirring was continued at room temperature over night. The reaction mixture was quenched with water and diluted with ethyl acetate. The organic phase was washed with saturated bicarbonate solution and brine, dried over sodium sulfate, filtered and evaporated. The crude yellow oil was chromatographed over silica gel gel (cyclohexane:ethyl acetate 83:17) to provide the title compound as a clear oil.

TLC: Rf=0.57 (5:1 cyclohexane:ethyl acetate);

¹H-NMR (360 MHz, CDCl₃): δ 8.25 (d, 1H), 7.82 (d, 1H), 4.48 (d, 2H), 3.51 (s, 3H).

### c) 5-Chloro-3-methoxymethyl-pyridine-2-carbonitrile

To a mixture of 2,5-dichloro-3-methoxymethyl-pyridine (1150 mg, 5.99 mmol) zinc cyanide (492 mg, 4.19 mmol) and zinc dust (39.2 mg, 0.599 mmol) in dry DMF (18 ml) was added (dppf)PdCl₂ CH₂Cl₂ adduct catalyst (245 mg, 0.299 mmol) under nitrogen. The mixture was heated at 150° C. for 2 hours. After 2 h the starting material was consumed and the reaction mixture was diluted with ethyl acetate and washed with saturated bicarbonate solution and brine, dried over sodium sulfate, filtered and evaporated. The crude dark residue (960 mg) was chromatographed over silica gel (cyclohexane:ethyl acetate 80:20) to provide the title compound as a yellow solid.

TLC: Rf=0.41 (3:1 cyclohexane:ethyl acetate);

LC-MS: Rt_{H9}=0.83 min. (100% pure, ESI+183, 185);

¹H-NMR (360 MHz, CDCl₃): δ 8.56 (d, 1H, H6), 7.95 (d, 1H, H4), 4.66 (s, 2H), 3.51 (s, 3H).

### d) 5-Chloro-3-methoxymethyl-pyridine-2-carboxylic acid

A suspension of 5-chloro-3-methoxymethyl-pyridine-2-carbonitrile (100 mg, 0.548 mmol) in 2N NaOH (2 ml) was stirred at 100° C. for 4 hours. The reaction mixture was washed with diethyl ether and then set acidic (pH 5-6) with 2M HCl. The aqueous layer was extracted with ethyl acetate and the organic phase washed with brine, dried over sodium sulfate, filtered and evaporated to provide the title compound as white solid.

MS: ESI-200; LC-MS: Rt_{H9}=0.58 min. (100% pure, ESI+202);

¹H-NMR (360 MHz, CDCl₃): δ 11.1 (s, broad, 1H, COOH), 8.45 (d, 1H, H6), 8.25 (d, 1H, H4), 4.99 (s, 2H), 3.55 (s, 3H).

### Acid-18: 3-Amino-5-prop-2-ynyloxy-pyrazine-2-carboxylic acid

The title compound was prepared by an analogous procedure to Acid-2 using prop-2-yn-1-ol instead of tetra-deutero methanol [Acid-2 step a)].

HPLC: Rt_{H9}=0.59 min; ESIMS [M+H]⁺=194.1;

¹H NMR (400 MHz, DMSO-d₆): δ 7.58 (br. s, 2H), 7.48 (s, 1H), 4.96 (d, 2H), 3.58 (s, 1H).

### Acid-19: 3-Chloro-1H-pyrrolo[2,3-b]pyridine-6-carboxylic acid a) 1H-Pyrrolo[2,3-b]pyridine-6-carbonitrile

To a mixture of 6-bromo-1H-pyrrolo[2,3-b]pyridine (Synthesis, 1992, 661, example 3b) (788 mg, 4 mmol), zinccyanide (329 mg, 2.80 mmol) and zinc dust (26.2 mg, 0.4 mmol) in dry DMF (12 ml) was added (dppf)PdCl₂×CH₂Cl₂ adduct catalyst (163 mg, 0.2 mmol) under nitrogen. The mixture was heated at 140° C. for 4 h. The reaction mixture was diluted with ethyl acetate and washed with aq. Saturated bicarbonate solution and brine, dried over sodium sulfate, filtered and evaporated. 1.04 g dark yellow oil. The crude product was chromatographed over silica gel (cyclohexane/ethyl acetate 3:1) to provide the title compound as a white solid.

TLC Rf=0.35 (2:1 cyclohexane:ethyl acetate);

LC-MS: Rt_{H11}=0.81 min. (100% purity, ESI+144), API-ES+ 144;

¹H-NMR (400 MHz, CDCl₃): δ 11.05 (s, 1H, NH), 8.08 (d, 1H), 7.71 (dd, 1H), 7.52 (d, 1H), 6.66 (m, 1H).

### b) 1H-Pyrrolo[2,3-b]pyridine-6-carboxylic acid

A suspension of 1H-pyrrolo[2,3-b]pyridine-6-carbonitrile (690 mg, 4.82 mmol) in NaOH 2M (12 ml) was stirred at 100° C. for 6 h. The reaction mixture was washed with diethyl ether and the aq. phase was set slightly acidic (pH 6-7) with conc. HCl. The solid formed was filtered off and dried to provide the title compound.

LC-MS: Rt_{H8}=0.51 min. (100% purity, ESI+163);

¹H-NMR (400 MHz, DMSO-D₆): δ 12.78 (s, 1H), 12.01 (s, 1H), 8.08 (d, 1H), 7.80 (m, 1H), 7.73 (s, broad, unresolved, 1H), 6.56 (s, broad, 1H).

### c) 3-Chloro-1H-pyrrolo[2,3-b]pyridine-6-carboxylic acid

A solution of 1H-pyrrolo[2,3-b]pyridine-6-carboxylic acid (300 mg, 1.85 mmol) and NCS (247 mg, 1.85 mmol) in dry DMF (12 ml) was stirred under argon at room temperature for 20 h. The reaction mixture was diluted with ethyl acetate and washed with brine. The precipitate formed was filtered off, washed with ethyl acetate and dried to provide the title compound as light brown solid.

LC-MS: Rt_{H8}=0.72 min. (100% purity, ESI+197/199);

¹H-NMR (400 MHz, DMSO-D₆): δ 13.03 (s, 1H), 12.43 (s, 1H), 8.07 (d, 1H), 7.96 (d, 1H), 7.90 (d, 1H).

### Acid-20: 3-(di-tert-Butoxycarbonyl-amino)-5-difluoromethyl-pyrazine-2-carboxylic acid

### a) 3-Amino-5-vinyl-pyrazine-2-carboxylic acid methyl ester

To a mixture of 161 mg (0.86 mmol) 3-amino-5-chloro-pyrazine-2-carboxylic acid methyl ester (GB 1248146), 0.352 ml (1.204 mmol) tributyl(vinyl)tin and 102 mg (2.498 mmol) lithium chloride in DMF was added 30.2 mg (0.043 mmol) PdCl₂(PPh₃)₂ and the mixture was heated to 85° C. for 2.5 h. After cooling to room temperature water was added and the mixture was extracted with EtOAc, the combined organic layers were washed with water and half saturated aq. NaCl, dried with Na₂SO₄ and evaporated. The residue was purified by chromatography on silica gel (cyclohexane to cyclohexane/EtOAc 1:9) to provide the title compound as yellow solid.

HPLC: Rt_{H11}=0.71 min; ESIMS [M+H]⁺=179.9;

¹H-NMR (600 MHz, DMSO-d₆): δ 8.04 (s, 1H), 7.35 (br. s, 1H), 6.75 (dd, 1H), 6.38 (d, 1H), 5.70 (d, 1H), 3.84 (s, 3H).

### b) 3-(di-tert-Butoxycarbonyl-amino)-5-vinyl-pyrazine-2-carboxylic acid methyl ester

To an ice cooled solution of 1.28 g (7.14 mmol) 3-amino-5-vinyl-pyrazine-2-carboxylic acid methyl ester in 45 ml DCM was added 8.58 g (39.3 mmol) Boc₂O and the mixture was stirred at room temperature for 30 min, then the mixture was heated to 40° C. for 4 h. After cooling to room temperature water was added and the mixture was extracted with DCM. The combined organic layers were washed with 0.5 N HCl and saturated aq. NaCl, dried with Na₂SO₄ and evaporated. The residue was purified by chromatography on silica gel (cyclohexane+5% NEt₃ to EtOAc+5% NEt₃) to provide the title compound as yellow solid.

HPLC: Rt_{H9}=1.15 min; ESIMS [M-Boc]⁺=280.3;

¹H NMR (400 MHz, DMSO-d₆): δ 8.93 (s, 1H), 7.00 (dd, 1H), 6.51 (dd, 1H), 5.86 (dd, 1H), 3.88 (s, 3H), 1.34 (s, 18H).

### c) 3-(di-tert-Butoxycarbonyl-amino)-5-formyl-pyrazine-2-carboxylic acid methyl ester

A mixture of 1 g (2.64 mmol) 3-(di-tert-butoxycarbonyl-amino)-5-vinyl-pyrazine-2-carboxylic acid methyl ester and 0.332 g (3.95 mmol) sodium bicarbonate in 45 ml DCM and 15 ml MeOH was cooled to −78° C. and purged with oxygen for 5 min. The reaction mixture was treated with ozone for 40 min until the mixture turned blue. The reaction mixture was purged with oxygen for 10 min and with nitrogen for 10 min, then 0.487 ml (6.59 mmol) dimethyl sulfide was added at −78° C. and the mixture was allowed to warm to room temperature. The mixture was diluted with DCM and washed with 10% aq. sodium thiosulfate. The aq. layer was extracted with DCM and the combined organic layers were dried with Na₂SO₄ and evaporated to provide the title compound as yellow oil. The compound was used for the next step without further purification.

¹H-NMR (400 MHz, DMSO-d₆): δ 10.07 (s, 1H), 9.24 (s, 1H), 3.94 (s, 3H), 1.36 (s, 18H).

### d) 3-(di-tert-Butoxycarbonyl-amino)-5-difluoromethyl-pyrazine-2-carboxylic acid methyl ester

To an ice cooled solution of 550 mg (1.44 mmol) 3-(di-tert-butoxycarbonyl-amino)-5-formyl-pyrazine-2-carboxylic acid methyl ester in 20 ml DCM was added dropwise within 1 h 0.798 ml (4.33 mmol) Deoxofluor (50% in THF). Stirring was continued at 0° C. for 2.5 h then the reaction mixture was allowed to room temperature over night. Saturated aq. sodium bicarbonate was added and the mixture extracted with EtOAc, the combined organic layers were washed with sat. aq. sodium chloride, dried with Na₂SO₄ and evaporated. The residue was purified by chromatography on silica gel (cyclohexane+5% NEt₃ to cyclohexane+5% NEt₃/EtOAc+5% NEt₃ 1:1) to provide the title compound as colorless solid.

HPLC: Rt_{H9}=1.14 min; ESIMS [2M+Na]⁺=829.6;

¹H-NMR (600 MHz, DMSO-d₆): δ 9.14 (s, 1H), 7.26 (t, 1H, CHF2), 3.92 (s, 3H), 1.33 (s, 18H).

### e) 3-(di-tert-Butoxycarbonyl-amino)-5-difluoromethyl-pyrazine-2-carboxylic acid

To a solution of 75 mg (0.186 mmol) 3-(di-tert-butoxycarbonyl-amino)-5-difluoromethyl-pyrazine-2-carboxylic acid methyl ester in 2 ml THF was added dropwise 0.205 ml (0.205 mmol) 1N NaOH and the reaction mixture was stirred for 1.5 h. To the mixture were added 0.186 ml (0.186 mmol) 1N HCl after stirring for 5 min toluene was added and the solvents were evaporated to provide the title compound together with sodium chloride as colorless solid. The mixture was used for coupling reactions without further purification.

HPLC: Rt_{H11}=0.89 min; ESIMS [M-Boc]⁺=290.0;

¹H-NMR (400 MHz, DMSO-d₆): δ 14.30 (br. s, 1H), 9.10 (s, 1H), 7.25 (t, 1H, CHF2), 1.33 (s, 18H).

### Acid-21: 5-Methoxy-3-methyl-pyridine-2-carboxylic acid

### a) 5-Methoxy-3-methyl-pyridine-2-carbonitrile

To a solution of 5-hydroxy-3-methyl-pyridine-2-carbonitrile (CAS registry 228867-86-5) (1.5 g, 11.18 mmol) and methanol (0.499 ml, 0.394 g, 12.30 mmol) in THF (100 ml) was added at 0° C. triphenylphosphine (4.44 g, 16.77 mmol) and the reaction mixture was stirred for 10 min at 0° C. Then a solution of DIAD (3.25 ml, 3.39 g, 16.77 mmol) in THF (50 ml) was added. The reaction mixture was stirred for 18 h at rt, diluted with EtOAc and washed with water and brine. The combined aq. layers were reextracted with EtOAc, the combined organic layers dried over Na₂SO₄, filtered and the filtrate was concentrated. The title compound was obtained after flash chromatography on silica gel (cyclohexane/EtOAc gradient 0-5 min 95:5, 5-50 min 95:5 to 60:40).

HPLC: Rt_{H10}=0.75 min; ESIMS: 149 [(M+H)⁺];

¹H NMR (400 MHz, CDCl₃): δ 8.22 (d, 1H), 7.08 (d, 1H), 3.92 (s, 3H), 2.55 (s, 3H).

### b) 5-Methoxy-3-methyl-pyridine-2-carboxylic acid

A solution of 5-methoxy-3-methyl-pyridine-2-carbonitrile (3.41 g, 10.20 mmol) in conc. aq. HCl soln. (10 ml) was stirred for 3.5 h at 120° C. The reaction mixture was cooled to rt, diluted with TBME and extracted twice with water. The combined aq. layers were washed with TBME and lyophilized. The residue was dissolved in water, 1M aq. NaOH soln. was added to adjust the pH to 3 and the solution was extracted 3× with DCM. The combined organic layers were dried over Na₂SO₄, filtrated and the filtrate was concentrated to yield the title compound as a white solid which was used for the next step without further purification.

HPLC: Rt_{H10}=0.40 min; ESIMS: 168 [(M+H)⁺];

¹H NMR (400 MHz, MeOD): δ 8.14 (d, 1H), 7.36 (d, 1H), 3.94 (s, 3H), 2.65 (s, 3H).

### Acid-22: 5-Difluoromethyl-3-methyl-pyridine-2-carboxylic acid

### a) 2-Chloro-5-difluoromethyl-3-methyl-pyridine

To a precooled solution of 6-chloro-5-methyl-pyridine-3-carbaldehyde (CAS registry 176433-43-5) (500 mg, 3.21 mmol) in DCM (15 ml) was added at −78° C. DAST (0.632 ml, 0.777 g, 4.82 mmol). The reaction mixture was stirred for 18 h at −78° C. to rt, then quenched at 0° C. with sat. aq. NaHCO₃ soln., diluted with H₂O and extracted with DCM. The organic layer was washed with H₂O, dried over Na₂SO₄, filtrated and the filtrate was concentrated. The title compound was obtained as a yellow oil after flash chromatography on silica gel (cyclohexane/EtOAc gradient 0-5 min 100:0, 5-40 min 100:0 to 80:20).

HPLC: Rt_{H10}=0.94 min; ESIMS: 178 [(M+H)⁺];

¹H NMR (400 MHz, CDCl₃): 8.38 (d, 1H), 7.72 (d, 1H), 6.69 (t, 1H), 2.46 (s, 3H).

### b) 5-Difluoromethyl-3-methyl-pyridine-2-carbonitrile

A solution of 2-chloro-5-difluoromethyl-3-methyl-pyridine (337 mg, 1.898 mmol), Zn(CN)₂ (159 mg, 1.328 mmol) and Pd(PPh₃)₄ (132 mg, 0.114 mmol) in DMF (10 ml) was stirred for 10 min at 120° C. in a microwave, filtrated over hyflo and washed with water and brine. The combined aq. layers were reextracted with TBME, the combined org. layers were dried over Na₂SO₄, filtrated and the filtrate was concentrated. The title compound was obtained as a yellow oil after flash chromatography on silica gel (cyclohexane/EtOAc gradient 0-3 min 100:0, 3-35 min 100:0 to 80:20).

HPLC: Rt_{H10}=0.83 min; ESIMS: 169 [(M+H)⁺];

¹H NMR (400 MHz, CDCl₃): δ 8.68 (s, 1H), 7.84 (s, 1H), 6.75 (t, 1H), 2.65 (s, 3H).

### c) 5-Difluoromethyl-3-methyl-pyridine-2-carboxylic acid

A solution of 5-difluoromethyl-3-methyl-pyridine-2-carbonitrile (209 mg, 0.787 mmol) in conc. aq. HCl soln. (2 ml) was stirred for 2 h at 120° C. in a sealed tube. The reaction mixture was cooled to rt, diluted with TBME and extracted twice with water. The combined aq. layers were washed with TBME and lyophilized. The residue was dissolved in water, 1M aq. NaOH soln. was added to adjust the pH to 2 and the solution was extracted 3× with DCM. The combined organic layers were dried over Na₂SO₄, filtrated and the filtrate was concentrated to yield the title compound as a white solid which was used for the next step without further purification.

HPLC: Rt_{H10}=0.49 min; ESIMS: 188 [(M+H)⁺];

¹H NMR (400 MHz, MeOD): δ 8.62 (s, 1H), 7.98 (s, 1H), 6.95 (t, 1H), 2.65 (s, 3H).

### Acid-23: 5-Fluoro-3-trideuteromethoxymethyl-pyridine-2-carboxylic acid

### a) 2-Chloro-5-fluoro-3-trideuteromethoxymethyl-pyridine

To a solution of (2-chloro-5-fluoropyridin-3-yl)-methanol (CAS: 870063-2-8; 950 mg, 5.88 mmol) in dry DMF (25 ml) was added sodium hydride (235 mg, 5.88 mmol, 60% in oil) at 0° C. After 15 minutes iodomethane-D3 (1.11 g, 7.64 mmol) was added and stirring was continued at room temperature for 4 h. The reaction mixture was quenched with water and diluted with ethyl acetate. The organic phase was washed with saturated bicarbonate solution and brine, dried over sodium sulfate, filtered and evaporated. The crude brown oil was chromatographed over silica gel (cyclohexane:ethyl acetate) to provide the title compound.

LC-MS: Rt_{H8}=0.87 min. (100% purity, ESI+179, 181);

¹H-NMR (360 MHz, CDCl₃): 8.18 (d, 1H), 7.64 (m, 1H), 4.52 (s, 2H).

### b) 5-Fluoro-3-trideuteromethoxymethyl-pyridine-2-carbonitrile

2-Chloro-5-fluoro-3-trideuteromethoxymethyl-pyridine (700 mg, 3.92 mmol) was reacted with zinc dust, zinccyanide and (dppf)PdCl₂ catalyst in an analogous manner as in example A1 c) to afford the title compound after silica gel chromatography (cyclohexane/ethyl acetate) to provide the title compound.

TLC Rf=0.42 (3:1 cyclohexane:ethyl acetate);

LC-MS: Rt_{H8}=0.74 min. (100% purity); API ES+ 170;

¹H-NMR (360 MHz, CDCl₃): δ 8.49 (d, 1H), 7.72 (m, 1H), 4.71 (s, 2H).

### c) 5-Fluoro-3-trideuteromethoxymethyl-pyridine-2-carboxylic acid

5-Fluoro-3-trideuteromethoxymethyl-pyridine-2-carbonitrile (150 mg, 0.887 mmol) was hydrolised in 2N NaOH in an analogous manner as in Acid 17 step d) to afford the crude title compound.

LC-MS: Rt_{H8}=0.58 min; (100% purity, ESI+189); API ES+ 189;

¹H-NMR (360 MHz, CDCl₃): δ 11.3 (broad, 1H), 8.36 (d, 1H), 8.01 (m, 1H), 5.03 (s, 2H).

### Acid-24: 5-Trideuteromethoxy-3-trideuteromethoxymethyl-pyridine-2-carboxylic acid

### a) 5-Trideuteromethoxy-3-trideuteromethoxymethyl-pyridine-2-carbonitrile

To a solution of CD3OD (48 mg, 1.33 mmol) in DMSO (2 ml) was added sodium hydride (53.2 mg, 1.33 mmol, 60% in oil) followed 10 minutes later by 5-Fluoro-3-trideuteromethoxymethyl-pyridine-2-carbonitrile (150 mg, 0.887 mmol, Acid-8 b)). The reaction mixture was heated at 90° C. for 1 h. The reaction mixture was diluted with ethyl acetate and washed with water and brine. The organic layer was dried over sodium sulfate, filtered and evaporated in vacuo. The crude product was chromatographed over silica gel (cyclohexane/ethyl acetate) to provide the title compound.

TLC: Rf=0.21 (3:1 cyclohexane:ethyl acetate);

LC-MS: Rt_{H8}=0.73 min, (93% purity, ESI+185); API-ES+ 185;

¹H-NMR (400 MHz, CDCl₃): δ 8.29 (d, 1H), 7.39 (d, 1H), 4.68 (s, 2H).

### b) 5-Trideuteromethoxy-3-trideuteromethoxymethyl-pyridine-2-carboxylic acid

5-Trideuteromethoxy-3-trideuteromethoxymethyl-pyridine-2-carbonitrile (80 mg, 0.434 mmol) was hydrolised in NaOH 2N (2 ml) in an analogous manner as in Acid 17 step d) to afford the crude title compound.

LC-MS: Rt_{H8}=0.49 min; (100% purity, ESI+204); API ES+ 204;

¹H-NMR (360 MHz, CDCl₃): δ 8.15 (d, 1H), 7.70 (d, 1H), 5.03 (s, 2H).

### Acid-25: 3-Amino-5-cyano-pyridine-2-carboxylic acid

### a) 5-Bromo-3-nitro-pyridine-2-carboxylic acid tert-butyl ester

To an ice cooled solution of 4.84 g (19.59 mmol) 5-bromo-3-nitro-pyridine-2-carboxylic acid (CAS 954240-89-2) in 59 ml THF was added 239 mg (1.96 mmol) DMAP and 5.56 g (25.5 mmol) Boc₂O and the reaction mixture was heated to 60° C. for 3 h. After cooling to 0° C. half saturated aq. sodium bicarbonate was added and the mixture extracted with EtOAc. The combined organic layers were washed with water and half saturated aq. NaCl, dried with Na₂SO₄ and evaporated. The residue was purified by chromatography on silica gel (cyclohexane to cyclohexane/EtOAc 3:2) to provide the title compound as pale beige solid.

HPLC: Rt_{H8}=1.17 min; ESIMS [M+H]⁺=304.1;

¹H-NMR (600 MHz, DMSO-d₆): δ 9.11 (s, 1H), 8.92 (s, 1H), 1.53 (s, 9H).

### b) 5-Cyano-3-nitro-pyridine-2-carboxylic acid tert-butyl ester

To a solution of 888 mg (2.93 mmol) 5-bromo-3-nitro-pyridine-2-carboxylic acid tert-butyl ester in 8.8 ml DMF was added 206 mg (1.76 mmol) zinc cyanide and 2 mg (0.03 mmol) zinc dust. The mixture was purged with nitrogen (3 times) 150 mg (0.293 mmol) bis(tri-tert-butylphosphine)palladium(0) were added and the mixture was heated to 80° C. for 4 h. After cooling to 0° C. water was added and the mixture extracted with EtOAc, the combined organic layers were washed with half saturated aq. NaCl, dried with Na₂SO₄ and evaporated. The residue was purified by chromatography on silica gel (cyclohexane to cyclohexane/EtOAc 1:4) to provide the title compound as beige solid.

HPLC: Rt_{H8}=1.04 min; ESIMS [M+H]⁺=248.0;

¹H-NMR (600 MHz, DMSO-d₆): δ 9.39 (s, 1H), 9.29 (s, 1H), 1.55 (s, 9H).

### c) 3-Amino-5-cyano-pyridine-2-carboxylic acid tert-butyl ester

To a mixture of 130 mg (0.522 mmol) 5-cyano-3-nitro-pyridine-2-carboxylic acid tert-butyl ester in 3 ml water was added 0.149 ml (2.61 mmol) acetic acid, the mixture was stirred at room temperature for 20 min, 454 mg (2.61 mmol) sodium dithionite were added and stirring was continued for 23 h. Additional 182 mg (1.043 mmol) sodium dithionite were added and the reaction mixture stirred for an other 48 h. The mixture was extracted with DCM, the combined organic layers were washed with water and saturated aq. NaCl, dried with Na₂SO₄ and evaporated to provide the title compound as yellow solid. The product was used for the next step without further purification.

HPLC: Rt_{H9}=0.86 min; ESIMS [M+H]⁺=220.2;

¹H-NMR (400 MHz, DMSO-d₆): δ 8.15 (d, 1H), 7.61 (d, 1H), 6.95 (br. s, 2H), 1.55 (s, 9H).

### d) 3-Amino-5-cyano-pyridine-2-carboxylic acid

To a mixture of 60 mg (0.274 mmol) 3-amino-5-cyano-pyridine-2-carboxylic acid tert-butyl ester and 0.358 ml (2.74 mmol) 1,3-dimethoxybenzene were added dropwise within 10 min 0.59 ml (7.66 mmol) TFA and the reaction mixture was stirred for 6 h. Toluene was added and the solvents were evaporated to provide the title compound as yellow solid. The product was used for the next step without further purification.

HPLC: Rt_{H9}=0.38 min; ESIMS [M+H]⁺=164.1;

¹H-NMR (400 MHz, DMSO-d₆): δ 13.05 (br. s, 1H), 8.16 (d, 1H), 7.64 (d, 1H), 7.08 (br. s, 2H).

### Acid-26: 3-Amino-5-difluoromethyl-pyridine-2-carboxylic acid

### a) 5-Difluoromethyl-3-nitro-pyridine-2-carboxylic acid tert-butyl ester

The title compound was prepared by an analogous reaction sequence to Acid-5 using 5-bromo-3-nitro-pyridine-2-carboxylic acid instead of 3-amino-5-chloro-pyrazine-2-carboxylic acid methyl ester in step a) and omitting step b).

HPLC: Rt_{H9}=1.07 min; ESIMS [M+H]⁺=275.3;

¹H NMR (600 MHz, DMSO-d₆): δ 9.18 (s, 1H), 8.82 (s, 1H), 7.31 (t, 1H, CHF2), 1.55 (s, 9H).

### b) 5-Difluoromethyl-3-nitro-pyridine-2-carboxylic acid

In a mixture of 5 ml DCM and 2.5 ml TFA was dissolved 345 mg (1.26 mmol) 5-difluoromethyl-3-nitro-pyridine-2-carboxylic acid tert-butyl ester and the reaction mixture was stirred for 4 h. Toluene was added and the solvents were evaporated to provide the title compound as colorless solid.

HPLC: Rt_{H9}=0.31 min; ESIMS [2M−H]⁻=435.3;

¹H-NMR (600 MHz, DMSO-d₆): δ 14.59 (br. s, 1H), 9.16 (s, 1H), 8.80 (s, 1H), 7.31 (t, 1H, CHF2).

### c) 3-Amino-5-difluoromethyl-pyridine-2-carboxylic acid

To a solution of 265 mg (1.22 mmol) 5-difluoromethyl-3-nitro-pyridine-2-carboxylic acid in EtOH was added 50 mg Raney-Nickel (Degussa B113W) and the reaction mixture was kept shaking under a hydrogen atmosphere for 16 h. The catalyst was filtered off (Celite) and washed with EtOH and the filtrate was evaporated to provide the title compound as off-white solid.

HPLC: Rt_{H9}=0.34 min; ESIMS [M+H]⁺=189.2;

¹H-NMR (600 MHz, DMSO-d₆): δ 7.98 (s, 1H), 7.39 (s, 1H), 7.09 (t, 1H, CHF2), 7.02 (br. s, 2H).

### Acid-27: 3-Chloro-5-difluoromethoxy-pyridine-2-carboxylic acid

### a) 3-Chloro-5-difluoromethoxy-pyridine-2-carbonitrile

To a solution of 3-chloro-5-hydroxy-pyridine-2-carbonitrile (330 mg, 2.03 mmol) in DMF (10 ml) was added K₂CO₃ (1.68 g, 12.2 mmol) and sodium chlorodifluoroacetate (1.29 g, 8.1 mmol) and the reaction mixture was heated at 100° C. for 10 min. The cold reaction mixture was diluted with TBME and washed with water and brine, dried over MgSO₄, filtered and concentrated. The title compound was obtained after flash column chromatography on silica gel (hexane to hexane-EtOAc 1:1) as a yellow oil: TLC (hexane-EtOAc 2:1): Rf=0.54;

HPLC: Rt_{H5}=0.966 min; ESIMS: 203, 205 [(M−H)⁻];

¹H NMR (360 MHz, CDCl₃): δ 8.41 (d, 1H), 7.61 (d, 1H), 6.60 (t, 1H).

### b) 3-Chloro-5-difluoromethoxy-pyridine-2-carboxylic acid

To a solution of 3-chloro-5-difluoromethoxy-pyridine-2-carbonitrile (470 mg, 2.29 mmol) in dioxane (18 ml) was added 1N NaOH (8.0 ml, 8 mmol) and the reaction mixture was stirred overnight at 70° C. The cold reaction mixture was acidified with 4N HCl and evaporated to dryness. The residue was suspended in CH₂Cl₂-MeOH 8:1, filtered and concentrated to provide the title compound as a yellow oil.

HPLC: Rt_{H5}=0.664 min; ESIMS: 222, 224 [(M−H)⁻];

¹H NMR (360 MHz, CDCl₃): δ 8.38 (br s, 1H), 7.82 (d, 1H), 7.06 (t, 1H).

### Acid 28: 5-cyano-3-methyl-pyridine-2-carboxylic acid

### a) 5-Bromo-3-methyl-pyridine-2-carboxylic acid tert-butyl ester

To a solution of 10.20 g (47.2 mmol) 5-bromo-3-methyl-pyridine-2-carboxylic acid and 20.61 g (94 mmol) di-tert-butyldicarbonate in 100 ml THF were added 0.577 g DMAP. Evolution of CO₂ started immediately and the mixture was stirred for 2 h at RT. TBME and sat aq NaHCO₃ were added. The layers were separated and the organic layer washed with sat aq NaHCO₃ and brine, and dried with MgSO₄.H₂O. Chromatography on silica gel (hexanes/EtOAc 1-7%) provided the title compound as a yellow liquid.

HPLC: Rt_{H3}=3.018 min; ESIMS [M+H]⁺=272, 274 (1Br); ¹H-NMR (360 MHz, CDCl₃): δ 8.59 s, 1H), 7.77 (s, 1H), 2.52 (s, 3H), 1.65 (s, 9H).

### b) 5-Bromo-3-methyl-pyridine-2-carboxylic acid tert-butyl ester

A mixture of 6.0 g (22.05 mmol) 5-bromo-3-methyl-pyridine-2-carboxylic acid tert-butyl ester, 1.813 g (15.43 mmol) Zn(CN)₂, 0.144 g Zn powder (2.205 mmol) and 0.571 g (0.551 mmol) Pd₂(dba)₃.CHCl₃ were suspended in 10 ml DMF under nitrogen atmosphere. tBu₃P (0.321 ml, 1.323 mmol) was added and the mixture was stirred for 5 h at 60° C. After being cooled down the mixture was diluted with TBME, filtered over celite and washed with brine three times. The crude product was purified by column chromatography on silica gel (hexanes/EtOAc 5-15%) to give the title compound as an off white solid. TLC (hexanes/EtOAc 3:1): Rf=0.31; HPLC: Rt_{H3}=2.431 min; ESIMS [M+Na]⁺=241; ¹H-NMR (360 MHz, CDCl₃): δ 8.78 (s, 1H), 7.88 (s, 1H), 2.56 (s, 3H), 1.67 (s, 9H); Ft-IR: 2231 cm⁻¹ (CN).

### c) 5-cyano-3-methyl-pyridine-2-carboxylic acid

To a solution of 8.50 g (38.9 mmol) 5-cyano-3-methyl-pyridine-2-carboxylic acid tert-butyl ester in 51 ml (389 mmol) 1,3-dimethoxybenzene were added 85 ml TFA and stirred for 6.5 h. The reaction mixture was diluted with toluene and evaporated. The residue was taken up in toluene and evaporated (2×). The product was crystallized from TBME/hexanes to give the title compound as a white powder. HPLC: Rt_{H1}=2.314 min;

ESIMS [M+Na]⁺=163; ¹H-NMR (360 MHz, CDCl₃): δ 8.77 (s, 1H), 8.07 (s, 1H), 2.87 (s, 3H).

### Acid-29: 3-Chloro-5-trideutero-methoxy-pyridine-2-carboxylic acid

### a) 3-Chloro-5-hydroxy-pyridine-2-carbonitrile

To an argon degased solution of acetic acid 5,6-dichloro-pyridin-3-yl ester (4.87 g, 23.66 mmol) in DMF (50 ml) was added Zn(CN)₂ (1.278 g, 10.88 mmol), zinc-dust (0.07 g, 1.06 mmol) and DPPF PdCl₂ (0.996 g, 1.18 mmol) and the resulting reaction mixture was heated at 150° C. for 18 h. The reaction mixture was diluted with TBME and water, filtered over Celite and the product was extracted with TBME. Combined extracts were washed with brine, dried over MgSO₄, filtered and concentrated. The title compound was obtained after re-crystallization from EtOAc-hexane as a beige solid: TLC (CH₂Cl₂-MeOH 19:1): Rf=0.22; HPLC: Rt_{H5}=0.677 min; ESIMS: 153,155 [(M−H)⁻];

¹H NMR (360 MHz, CD₃OD): δ 8.19 (d, 1H), 7.41 (d, 1H).

### b) 3-Chloro-5-trideutero-methoxy-pyridine-2-carbonitrile

To a solution of 3-chloro-5-hydroxy-pyridine-2-carbonitrile (0.855 g, 5.5 mmol) in THF (50 ml) was added at 0° C. CD₃OD (0.292 ml, 7.19 mmol) and PPh₃ (2.176 g, 8.30 mmol) and afterwards dropwise DIAD (1.613 ml, 8.30 mmol). After stirring for 1 h at 0-5° C. the reaction mixture was concentrated. The title compound was obtained after flash column chromatography on silica gel (toluene-EtOAc 3:1) as a colorless solid: TLC (toluene-EtOAc 1:1): Rf=0.57;

HPLC: Rt_{H5}=0.866 min; ESIMS: 172, 174 [(M+H)⁺]; ¹H NMR (360 MHz, CDCl₃): δ 8.30 (d, 1H), 7.31 (d, 1H).

### c) 3-Chloro-5-trideutero-methoxy-pyridine-2-carboxylic acid

To a solution of 3-chloro-5-trideutero-methoxy-pyridine-2-carbonitrile (760 mg, 4.43 mmol) in dioxane (10 ml) was added 4N NaOH (11.07 ml, 44.3 mmol) and the reaction mixture was stirred overnight at 85° C. The cold reaction mixture was acidified with 4N HCl and extracted with EtOAc. Combined extracts were washed with brine, dried over MgSO₄, filtered and concentrated. The title compound was obtained after crystallization from EtOAc-diisopropylether as a colorless solid.

HPLC: Rt_{H5}=0.538 min; ESIMS: 191,193 [(M+H)⁺];

¹H NMR (360 MHz, CDCl₃): δ 8.21 (d, 1H), 7.38 (d, 1H).

### Acid-30: Sodium; 4-difluoromethyl-6-methoxy-pyridazine-3-carboxylate

### a) 2-Diazo-4,4-difluoro-3-oxo-butyric acid ethyl ester

To a solution of 4,4-difluoro-3-oxo-butyric acid ethyl ester (5.0 g, 29 mmol) and 4-acetylamino-benzenesulfonyl azide (7.95 g, 32 mmol) in ACN (50 mL) was added at 0° C. NEt₃ (6.1 mL, 43.8 mmol) within 30 min. The reaction mixture was stirred for 2 h at 0-5° C. and overnight at 25° C., than diluted with TBME and filtered. The filtrate was washed with 10% aq. NaH₂PO₄ and brine, dried over MgSO₄, filtered and concentrated. The title compound was obtained after flash column chromatography on silica gel (hexane to hexane-TBME 1:1) as a yellow oil.

TLC (hexane-TBME 1:1): Rf=0.46;

¹H NMR (360 MHz, CDCl₃): δ 6.62 (t, 1H), 4.38 and 4.24 (q, 2H), 1.38 and 1.31 (t, 3H).

### b) (E)-4-Diazo-3-difluoromethyl-pent-2-enedioic acid 5-ethyl ester 1-methyl ester

To a solution of 2-diazo-4,4-difluoro-3-oxo-butyric acid ethyl ester 0.5 g, 2.6 mmol) in Et₂O (10 mL) was added methoxycarbonylmethylen-triphenylphosphoran (1.3 g, 3.9 mmol) and the reaction mixture was stirred for 3 days at 25° C. The reaction mixture was filtered through a plug of silica gel and concentrated to provide the title compound after purification by flash column chromatography on silica gel (hexane to hexane-TBME 1:1) as a yellow oil.

TLC (hexane-TBME 1:1): Rf=0.60;

¹H NMR (360 MHz, CDCl₃): δ 6.82 (t, 1H), 6.32 (s, 1H), 4.29 (q, 2H), 3.79 (s, 3H), 1.34 (t, 3H).

### c) 4-Difluoromethyl-6-methoxy-pyridazine-3-carboxylic acid ethyl ester

To a solution of (E)-4-diazo-3-difluoromethyl-pent-2-enedioic acid 5-ethyl ester 1-methyl ester (0.18 g, 0.78 mmol) in Et₂O (10 ml) was added PPh₃ (0.31 g, 1.18 mmol) and the reaction mixture was stirred for 3 days at 25° C. The reaction mixture was concentrated and purified by flash column chromatography on silica gel (hexane to hexane-TBME 1:1) to obtain the title compound as a yellow oil.

TLC (hexane-TBME 1:1): Rf=0.31;

HPLC: Rt_{H5}=0.877 min;

¹H NMR (360 MHz, CDCl₃): δ 7.41 (t, 1H), 7.13 (s, 1H), 4.52 (q, 2H), 4.25 (s, 3H), 1.45 (t, 3H).

### d) Sodium; 4-difluoromethyl-6-methoxy-pyridazine-3-carboxylate

To a solution of 4-difluoromethyl-6-methoxy-pyridazine-3-carboxylic acid ethyl ester (0.13 g, 0.56 mmol) in dioxane (2 ml) was added 4N NaOH (0.7 ml, 2.8 mmol) and the reaction mixture was stirred for 0.5 h at 25° C. After addition of 4N HCl (0.56 mL, 2.24 mmol) the reaction mixture was evaporated to dryness. The crude product was re-dissolved in DMF and concentrated again to provide the title compound as a light yellow solid, which was used as such in the next step.

HPLC: Rt_{H5}=0.420 min; ESIMS: 203 [(M−H)⁻].

### Acid-31: 5-Cyano-3-trideuteromethoxymethyl-pyridine-2-carboxylic acid

### a) 5-Chloro-3-trideuteromethoxymethyl-pyridine-2-carboxylic acid

5-Chloro-3-trideuteromethoxymethyl-pyridine-2-carboxylic acid was prepared from (2,5-dichloro-pyridin-3-yl)-methanol in analogous manner to the sequence of Acid-1 step a) to d) using trideuteromethyliodide instead of methyliodide in the alkylation step b).

LC-MS: Rt_{H8}=0.77 min. (100% purity, ES+ 205, 207), API ES− 203, 205;

¹H-NMR (400 MHz, CDCl₃): δ 8.47 (d, 1H), 8.27 (m, 1H), 4.87 (s, 2H).

### b) 5-Chloro-3-trideuteromethoxymethyl-pyridine-2-carboxylic acid benzyl ester

A mixture of 5-Chloro-3-trideuteromethoxymethyl-pyridine-2-carboxylic acid (100 mg, 0.489 mmol) and 2-benzyl-1,3-dicyclohexyl-isourea (169 mg, 0.538 mmol) in toluene (2 ml) was stirred at 90° C. for 3. The reaction mixture was filtered and evaporated in vacuo. Chromatography over silica gel (cyclohexane/ethyl acetate) afforded the title compound.

LC-MS: Rt_{H8}=1.15 min. (100% purity, ES+ 295, 297);

¹H-NMR (400 MHz, CDCl₃): δ 8.85 (d, 1H), 8.10 (d, 1H), 7.50 (m, 2H), 7.40 (m, 3H), 5.46 (s, 2H), 4.82 (s, 2H).

### c) 5-Cyano-3-trideuteromethoxymethyl-pyridine-2-carboxylic acid benzyl ester

5-Chloro-3-trideuteromethoxymethyl-pyridine-2-carboxylic acid benzyl ester (120 mg, 0.407 mmol) was reacted with zinc dust (2.66 mg, 0.04 mmol), zinc cyanide (28.7 mg, 0.244 mmol) and bis(tri-t-butylphosphine)palladium(0) catalyst (20.81 mg, 0.041 mmol) in an analogous manner at 80° C. for 3 h as in Acid 17 step c) to afford the title compound after silica gel chromatography (cyclohexane/ethyl acetate). TLC Rf=0.40 (3:1 cyclohexane:ethyl acetate);

LC-MS: Rt_{H8}=1.04. (100%, ES+ 286);

¹H-NMR (400 MHz, CDCl₃): δ 8.87 (d, 1H), 8.39 (d, 1H), 7.50 (m, 2H), 7.40 (m, 3H), 5.47 (s, 2H), 4.82 (s, 2H).

### d) 5-Cyano-3-trideuteromethoxymethyl-pyridine-2-carboxylic acid

A solution of 5-Cyano-3-trideuteromethoxymethyl-pyridine-2-carboxylic acid benzyl ester (50 mg, 0.175 mmol) in ethanol (1.8 ml) was hydrogenated for 18 hours over Pd/C (10%, 18.65 mg) at room temperature and atmospheric pressure. The reaction mixture was filtered and evaporated in vacuo. The residue was partitioned between diethyl ether and 2N NaOH solution. The aqueous phase was set acidic with 2N HCl solution and was extracted with ethyl acetate. The organic phase was washed with brine, dried over sodium sulfate, filtered and concentrated in vacuo to provide the title compound as glassy solid.

LC-MS: Rt_{H8}=0.48 min. (100% purity, ES− 194);

¹H-NMR (400 MHz, CDCl₃): δ 8.81 (d, 1H), 8.60 (m, 1H), 5.05 (s, 2H).

### Acid-32: 3-Chloro-5-difluoromethyl-pyridine-2-carboxylic acid

### a) 5-Bromo-3-chloro-pyridine-2-carboxylic acid tert-butyl ester

To an ice cooled solution of 11.82 g (50 mmol) 5-bromo-3-chloro-pyridine-2-carboxylic acid (CAS 1189513-51-6) in 150 ml THF was added 611 mg (5 mmol) DMAP and 14.19 g (65 mmol) Boc₂O and the reaction mixture was heated to 60° C. for 3 h. After cooling to 0° C. half saturated aq. sodium bicarbonate was added and the mixture extracted with EtOAc. The combined organic layers were washed with half saturated aq. NaCl, dried with Na₂SO₄ and evaporated. The residue was purified by chromatography on silica gel (cyclohexane to cyclohexane/EtOAc 1:1) to provide the title compound as colorless oil.

HPLC: Rt_{H8}=1.22 min; ESIMS [M-tBu]⁺=237.8;

¹H-NMR (600 MHz, DMSO-d₆): δ 8.73 (d, 1H), 8.52 (d, 1H), 1.55 (s, 9H).

### b) 3-Chloro-5-vinyl-pyridine-2-carboxylic acid tert-butyl ester

A mixture of 1.755 g (6 mmol) 5-bromo-3-chloro-pyridine-2-carboxylic acid tert-butyl ester and 884 mg (6.6 mmol) potassium trifluoro(vinyl)borate in 18 ml dioxane was purged with nitrogen, 1.67 ml (12 mmol) triethylamine and 153 mg (0.3 mmol) bis(tri-tert-butylphosphine)palladium(0) were added and the mixture was heated to 80° C. for 0.5 h. After cooling to room temperature and addition of EtOAc the mixture was filtered through Hyflo and the filtrate was evaporated. The residue was purified by chromatography on silica gel (cyclohexane to cyclohexane/EtOAc 7:3) to provide the title compound as pale yellow oil.

HPLC: Rt_{H8}=1.13 min; ESIMS [M-tBu]⁺=184.0;

¹H-NMR (600 MHz, DMSO-d₆): δ 8.64 (s, 1H), 8.24 (s, 1H), 6.79 (dd, 1H), 6.18 (d, 1H), 5.56 (d, 1H), 1.55 (s, 9H).

### c) 3-Chloro-5-difluoromethyl-pyridine-2-carboxylic acid

The title compound was prepared by an analogous reaction sequence to Acid-5 steps c) and d) using 3-chloro-5-vinyl-pyridine-2-carboxylic acid tert-butyl ester instead of 3-(di-tert-butoxycarbonyl-amino)-5-vinyl-pyrazine-2-carboxylic acid methyl ester [Acid-5 step c)], followed by cleavage of the tert.-butyl ester in a manner analogous to the procedure of Acid-11 step b).

HPLC: Rt_{H9}=0.41 min; ESIMS [M+H]⁺=207.8;

¹H NMR (600 MHz, DMSO-d₆): 14.30 (br. s, 1H), 8.78 (s, 1H), 8.34 (s, 1H), 7.20 (t, 1H, CHF2).

### Acid-33: 3-Chloro-5-trideuteromethoxy-dideuteromethyl-pyrrolo[2,3-b]pyridin-6-carboxylic acid

### a) 1-Triisopropylsilanyl-1H-pyrrolo[2,3-b]pyridine-5-deuterocarbaldehyde

To a solution of 1-triisopropylsilanyl-1H-pyrrolo[2,3]pyridine-5-bromide (18.8 g, 53.2 mmol, CAS: 858116-66-2) was added dropwise butyllithium (23.4 ml, 58.5 mmol, 2.5 molar in hexane) at −78° C. under a nitrogen atmosphere. After stirring for 45 minutes at this temperature deutero-D1-DMF (6.26 ml, 80 mmol, 98% from Armar) in THF (5 ml) was added slowly and the cooling bath was removed 15 minutes after the addition was complete. The reaction was quenched at 0° C. by adding aq. 1N acetic acid (5 ml) and was diluted with ethyl acetate. The organic phase was washed with saturated sodium bicarbonate solution and brine, dried over sodium sulfate, filtered and evaporated. 17.1 g (94% yield). TLC Rf=0.55 (5:1 cyclohexane:ethyl acetate). LC-MS Rt_{H9}=1.54 min. (89% purity, ES+ 304), API MS ES+ 304. ¹H-NMR (400 MHz, CDCl₃): 8.79 (d, 1H), 8.40 (d, 1H), 7.42 (d, 1H), 6.72 (d, 1H), 1.89 (septett, 3H), 1.15 (d, 18H). Used crude in the next step.

### b) (1-Triisopropylsilanyl-1H-pyrrolo[2,3-b]pyridine-5-yl)-dideuteromethanol

To a solution of 1-triisopropylsilanyl-1H-pyrrolo[2,3]pyridine-5-deuterocarbaldehyde (17.1 g, 50.1 mmol) in ethanol (250 ml) was added sodium borodeuteride (2.6 g, 62.2 mmol) at room temperature. Stirring was continued for 2 h and the reaction was carefully quenched with 1N acetic acid. The reaction mixture was diluted with ethyl acetate and washed with sat. sodium bicarbonate solution and brine, dried over sodium sulfate, filtered and evaporated. 18 g oil. Silica gel chromatography (89:11 cychlohexane:ethyl acetate) afforded the title compound as a white solid. 12.55 g (82% yield). TLC Rf=0.46 (2:1 cyclohexane:ethyl acetate). LC-MS Rt_{H8}=1.40 min. (100% purity, ES+ 307). API MS ES+ 307. ¹H-NMR (400 MHz, CDCl₃): 8.29 (d, 1H), 7.91 (d, 1H), 7.35 (d, 1H), 6.57 (d, 1H), 1.88 (septett, 3H), 1.15 (d, 18H).

### c) 5-Trideuteromethoxy-dideuteromethyl-1-triisopropylsilanyl-1H-pyrrolo[2,3-b]pyridine

To a solution of (1-Triisopropylsilanyl-1H-pyrrolo[2,3-b]pyridine-5-yl)-dideuteromethanol (5 g, 16.31 mmol) in dry DMF (125 ml) was added sodium hydride (718 mg, 17.94 mmol, 60% in oil) at 0° C. After 15 minutes D3-methyl iodide (1.354 ml, 21.21 mmol) was added and stirring was continued at room temperature for 3 h. The reaction mixture was quenched with water and diluted with ethyl acetate. The organic phase was washed with sat. sodium bicarbonate solution and brine, dried over sodium sulfate, filtered and evaporated. 4 g yellow oil. Silica gel chromatography (85:15 cychlohexane:ethyl acetate) afforded the title compound. 3.235 g (61.3% yield). TLC Rf=0.55 (2:1 cyclohexane:ethyl acetate). LC-MS Rt_{H8}=1.68 min. (96% purity, ES+ 324). ¹H-NMR (400 MHz, CDCl₃): 8.26 (d, 1H), 7.88 (d, 1H), 7.33 (d, 1H), 6.56 (d, 1H), 1.88 (septett, 3H), 1.15 (d, 18H).

### d) 5-Trideuteromethoxy-dideuteromethyl-1H-pyrrolo[2,3-b]pyridine

To a solution of 5-trideuteromethoxy-dideuteromethyl-1-triisopropylsilanyl-1H-pyrrolo[2,3-b]pyridine (3.253 g, 10.05 mmol) in dry THF (20 ml) was added TBAF 1M in THF (10.56 ml, 10.56 mmol). The reaction was stirred for 18 h at room temperature. The reaction mixture was poured into water and extracted with ethyl acetate. The organic phase was washed with brine, dried over sodium sulfate, filtered and evaporated. 4.31 g. Silica gel chromatography (cyclohexane/ethyl acetate) afforded the title compound. 791 mg (47.1% yield). TLC Rf=0.13 (1:2 cyclohexane:ethyl acetate). LC-MS Rt_{H8}=0.54 min. (100% purity, ES+ 168). API MS ESI+168. ¹H-NMR (400 MHz, CDCl₃): 10.50 (broad s, 1H), 8.36 (d, 1H), 7.98 (d, 1H), 7.40 (m, 1H), 6.53 (m, 1H).

### e) 5-Trideuteromethoxy-dideuteromethyl-1H-pyrrolo[2,3-b]pyridine 7-oxide

To a solution of 5-Trideuteromethoxy-dideuteromethyl-1H-pyrrolo[2,3-b]pyridine (780 mg, 4.66 mmol) in DME (20 ml) was added m-CPBA (1127 mg, 6.53 mmol) at 0° C. Stirring was continued at room temperature over night. The solvent was removed in vacuo and the crude product was suspended in water and the pH was adjusted to 9 with sat. potassium carbonate solution. Stirring was continued and the aqueous solution was saturated with sodium chloride and extracted with ethyl acetate. The organic phase was dried over sodium sulfate, filtered and evaporated. 988 mg brown oil which was used without purification in the next step. LC-MS Rt_{H8}=0.52 min. (73% purity, ES+ 184). API MS ESI+ 184. ¹H-NMR (400 MHz, CDCl₃): 12.81 (broad s, 1H), 8.26 (d, 1H), 7.68 (d, 1H), 7.41 (m, 1H), 6.55 (m, 1H).

### f) (6-Bromo-5-trideuteromethoxy-dideuteromethyl-pyrrolo[2,3-b]pyridin-1-yl)-phenyl-methanone

Solutions of benzoyl bromide (1.319 ml, 11.19 mmol) in dichloromethane (2 ml) and HMDS (0.938 ml, 4.48 mmol) in dichloromethane (1 ml) were simultaneously added dropwise to a solution of 5-trideuteromethoxy-dideuteromethyl-1H-pyrrolo[2,3-b]pyridine 7-oxide (988 mg, 4.48 mmol) in dichloromethane (3 ml) under argon atmosphere at rt over 30 minutes. Stirring was continued at room temperature over night. White precipitation. After 18 h stirring the mixture was washed with sat. sodium bicarbonate solution and brine, dried over magnesium sulfate, filtered and evaporated. 652 mg brown oil. Silica gel chromatography (cyclohexane/EtOAc) afforded the title compound. 326 mg (21% yield). LC-MS Rt_{H8}=1.27 min. (90% purity, ES+ 350/352). ¹H-NMR (400 MHz, CDCl₃): 8.01 (s, 1H), 7.91 (dd, 2H), 7.77 (d, 1H), 7.64 (t, 1H), 7.53 (t, 2H), 6.56 (d, 1H).

### g) 6-Bromo-5-trideuteromethoxy-dideuteromethyl-pyrrolo[2,3-b]pyridin

To a solution of (6-Bromo-5-trideuteromethoxy-dideuteromethyl-pyrrolo[2,3-b]pyridin-1-yl)-phenyl-methanone (320 mg, 0.91 mmol) in methanol (8 ml) was added 2M NaOH solution (4.57 ml, 9.14 mmol) and the reaction mixture was stirred at rt for 2 days. The white solid formed in the reaction was filtered off and dried. 124 mg. The filtrate was evaporated in vacuo, diluted with ethyl acetate and washed with sat. sodium bicarbonate solution and brine. The organic layer was dried over sodium sulfate, filtered and evaporated. 55 mg. Combined solids: 179 mg (80% yield). LC-MS Rt_{H8}=0.84 min. (85% purity, ES+ 246/248). ¹H-NMR (400 MHz, CDCl₃): 10.78 (broad s, 1H), 8.06 (s, 1H), 7.44 (m, 1H), 6.53 (m, 1H).

### h) 6-Cyano-5-trideuteromethoxy-dideuteromethyl-pyrrolo[2,3-b]pyridin

A mixture of 6-Bromo-5-trideuteromethoxy-dideuteromethyl-pyrrolo[2,3-b]pyridin (120 mg, 0.488 mmol), zinc (0.319 mg, 4.88 μmol) and zinc cyanide (34.4 mg, 0.293 mmol) in dry DMF (1.5 ml) was degassed with argon in a 4 ml microwave vial for 20 minutes. Bis(tri-t-butylphosphine)palladium(0) (24.92 mg, 0.049 mmol) was added and the vial was sealed and heated at 80° C. for 4 h. The reaction mixture was poured into a water/ice/ethyl acetate mixture and the organic layer was washed twice with brine, dried over sodium sulfate, filtered and evaporated. Silica gel chromatography (cyclohexane/EtOAc) of the crude product (164 mg) afforded the title compound as a white solid. 71 mg (76% yield). LC-MS Rt_{H8}=0.73 min. (100% purity, ES+ 193). ¹H-NMR (400 MHz, CDCl₃): 10.14 (broad s, 1H), 8.15 (s, 1H), 7.66 (m, 1H), 6.64 (m, 1H).

### i) 5-Trideuteromethoxy-dideuteromethyl-pyrrolo[2,3-b]pyridin-6-carboxylic acid

A suspension of 6-cyano-5-trideuteromethoxy-dideuteromethyl-pyrrolo[2,3-b]pyridin (70 mg, 0.364 mmol) in 2M NaOH (2 ml) was stirred at 100° C. for 18 h. The reaction mixture was washed with diethyl ether and the aqueous layer was set acidic (pH 6-7) with 2M HCl solution. The white solid formed was filtered off and washed with water. 63 mg white solid. The filtrate was extracted with ethyl acetate, washed with brine, dried ofer sodium sulfate, filtered and evaporated. 12 mg white solid. Combined solids: 75 mg white solid (98% yield). LC-MS Rt_{H8}=0.60 min. (100% purity, ES+ 212). ¹H-NMR (400 MHz, CDCl₃): 8.48 (s, 1H), 7.60 (m, 1H), 6.67 (m, 1H).

### j) 3-Chloro-5-trideuteromethoxy-dideuteromethyl-pyrrolo[2,3-b]pyridin-6-carboxylic acid

To a solution of 5-Trideuteromethoxy-dideuteromethyl-pyrrolo[2,3-b]pyridin-6-carboxylic acid (70 mg, 0.331 mmol) in dry DMF (12 ml) was added NCS (44.3 mg, 0.331 mmol) and the reaction mixture was stirred at rt for 20 hours under argon. The reaction mixture was diluted with ethyl acetate and washed with brine. The precipitate formed was filtered off, washed with ethyl acetate and dried in vacuo. The residue (300 mg) was stirred in water and the insoluble part was filtered off affording 20 mg (25% yield) of a light yellow solid. LC-MS Rt_{H8}=0.78 min. (100% purity, ES− 244). ¹H-NMR (400 MHz, DMSO-D₆): 13.04 (s, 1H), 12.28 (broad s, 1H, NH), 8.06 (s, 1H), 7.91 (d, 1H).

### Acid-34: 5-Trideuteromethoxy-3-methyl-pyridine-2-carboxylic acid

5-Trideuteromethoxy-3-methyl-pyridine-2-carboxylic acid was prepared by an analogous reaction sequence to Acid-6 using CD₃OD instead of methanol in the first step; HPLC:

Rt_{H10}=0.42 min; ESIMS: 171 [(M+H)⁺];

¹H NMR (400 MHz, MeOD): δ 8.13 (d, 1H), 7.36 (d, 1H), 2.65 (s, 3H).

### Acid-35: 5-Fluoro-3-methyl-pyridine-2-carboxylic acid

### a) 5-Fluoro-3-methyl-pyridine-2-carbonitrile

A soln. of 2-chloro-5-fluoro-3-methyl-pyridine (CAS 38186-84-4, 408 mg, 2.750 mmol), Zn(CN)₂ (230 mg, 1.923 mmol) and Pd(PPh₃)₄ (190 mg, 0.165 mmol) in DMF (8 ml) was stirred at 120° C. for 0.5 h in a microwave. The reaction mixture was filtered over hyflo, diluted with TBME and H₂O and extracted with brine. The aq. phases were reextracted with TBME, the combined org. phases were dried over Na₂SO₄, filtered and concentrated. Flash chromatography on silica gel (hexane-EtOAc 100:0 to 80:20) yielded the title compound.

HPLC: Rt_{H12}=0.72 min; ¹H NMR (400 MHz, CDCl₃): δ 8.42 (d, 1H), 7.42-7.39 (m, 1H), 2.61 (s, 3H).

### b) 5-Fluoro-3-methyl-pyridine-2-carboxylic acid

5-Fluoro-3-methyl-pyridine-2-carbonitrile (254 mg, 1.866 mmol) in conc. aq. HCl (1.5 ml) was stirred in a sealed glass vial at 120° C. for 2 h. The reaction mixture was basified with solid NaOH and extracted twice with TBME. The combined org. phases were washed with H₂O. The combined aq. phases were acidified to pH 2 with 2M aq. HCl, and were three times extracted with TMBE, dried over Na₂SO₄, filtered and concentrated. The crude product was used in the next step without further purification.

HPLC: Rt_{H12}=0.48 min; ESIMS [M+H]⁺=156; ¹H NMR (400 MHz, CD₃OD): δ 8.37 (d, 1H), 7.63 (dd, 1H), 2.64 (s, 3H).

### Amide 1: 5-Cyano-3-methyl-pyridine-2-carboxylic acid amide

To a white suspension of 5-cyano-3-methyl-pyridine-2-carboxylic acid (84 mg, 0.518 mmol) in DCM (1.5 ml) was added oxalyl chloride (0.068 ml, 99 mg, 0.777 mmol) and a catalytic amount of DMF. The reaction mixture was stirred at rt for 1 h and was then added dropwise to a 25% aq. NH₄OH soln. (0.300 ml) at 0° C. The reaction mixture was stirred for 10 min at rt, H₂O and TBDME were added, the phases were separated and the aq. phase was twice reextracted with TBDME. The combined org. phases were dried over Na₂SO₄, filtered and concentrated to leave a white powder that was used in the next step without further purification. Rt_{H11}=0.47 min; ESIMS: 162 [(M+H)⁺]; ¹H NMR (400 MHz, DMSO-d₆): δ 8.68 (d, 1H), 7.91 (d, 1H), 7.80 (br s, 1H), 5.57 (br s, 1H), 2.80 (s, 3H).

### Catalyst 1: 3,5-Bis-trifluoromethyl-benzoic acid (S)-(6-hydroxy-quinolin-4-yl)-((1S,2R,4S,5R)-5-vinyl-1-aza-bicyclo[2.2.2]oct-2-yl)-methyl ester

### a) 3,5-Bis-trifluoromethyl-benzoic acid (S)-(6-triisopropylsilanyloxy-quinolin-4-yl)-((1S,2R,4S,5R)-5-vinyl-1-aza-bicyclo[2.2.2]oct-2-yl)-methyl ester

To a solution of (S)-(6-triisopropylsilanyloxy-quinolin-4-yl)-((1S,2R,4S,5R)-5-vinyl-1-aza-bicyclo[2.2.2]oct-2-yl)-methanol (Deng et al., J. Amer. Chem. Soc. 2006, 128, 732; CAS Nr.: 876269-55-5; 3.22 g, 6.90 mmol) and Et₃N (1.442 ml, 10.35 mmol) in DCM was added dropwise 3,5-bis-trifluoromethyl-benzoyl chloride (2.480 g, 8.97 mmol). TLC (hexanes/(EtOAc/MeOH 95:5) 1:1): Rf=0.44; HPLC: Rt_{H6}=3.256 min; ESIMS [M+H]⁺=707; ¹H-NMR (DMSO-d6, 360 MHz): δ 8.76 (d, 1H), 8.53 (s, 2H), 8.00 (d, 1H), 7.73 (d, 1H), 7.59 (s, 1H), 7.41 (d, 1H), 6.49 (d, 1H), 6.09 (ddd, 1H), 5.11 (d, 1H), 5.07 (d, 1H), 3.59 (q, 1H), 2.85-2.73 (m, 2H), 2.65-2.56 (m, 2H), 2.33-2.23 (m, 1H), 1.96-1.50 (m, 5H), 1.38-1.25 (m, 3H), 1.10 (d, 18H).

### b) 3,5-Bis-trifluoromethyl-benzoic acid (S)-(6-hydroxy-quinolin-4-yl)-((1S,2R,4S,5R)-5-vinyl-1-aza-bicyclo[2.2.2]oct-2-yl)-methyl ester

To a solution of compound catalyst 1a) (4.88 g, 6.90 mmol) in 50 ml THF was added dropwise HF-Py (1.8 ml, 68 mmol). The reaction was slightly exothermic and the resulting yellow solution was stirred at rt for 30 min. The mixture was diluted with EtOAc and washed with sat aq. NaHCO3 (3×) and brine. The org layer was dried with Na2SO4 and evaporated.

The crude product was purified by column chromatography on silica gel (hexanes/(EtOAc/MeOH 20:1) 30-75%) to give the title compound as a pale yellow solid. TLC (hexanes/(EtOAc/MeOH 5%] 1:3): Rf=0.28; HPLC: Rt_{H3}=2.464 min [M+H]⁺551; ¹H-NMR (DMSO-d6, 600 MHz): δ 10.20 (s, 1H), 8.72 (d, 1H), 8.58 (s, 2H), 8.51 (s, 1H), 7.89 (d, 1H), 7.61 (d, 1H), 7.49 (s, 1H), 7.33 (d, 1H), 6.49 (d, 1H), 6.07 (ddd, 1H), 5.10 (d, 1H), 5.07 (d, 1H), 3.50-3.43 (m, 1H), 2.88-2.73 (m, 2H), 2.67-2.50 (m, 2H), 2.23 (q, 1H), 1.93-1.83 (m, 1H), 1.78 (s, 1H), 1.70-1.61 (m 1H), 1.60-1.52 (m, 1H), 1.50-1.44 (m, 1H).

### Example 275

### Biological Activity of Compounds of the Invention

The compounds of the Examples hereinbefore show the following IC₅₀ values in the assay for inhibition of BACE-2 described hereinbefore:

TABLE 31

| | BACE-2 IC50 |
|---|---|
| Example | [μM] |
| 1 | 0.061 |
| 2 | 0.087 |
| 3 | 0.056 |
| 4 | >10.000 |
| 5 | 0.373 |
| 6 | 3.444 |
| 7 | 0.309 |
| 8 | 7.231 |
| 9 | 0.663 |
| 10 | 0.289 |
| 11 | 1.057 |
| 12 | 2.260 |
| 13 | 0.431 |
| 14 | 0.335 |
| 15 | 6.592 |
| 16 | 0.000 |
| 17 | 0.694 |
| 18 | 1.657 |
| 19 | 3.180 |
| 20 | 0.226 |
| 21 | 0.069 |
| 22 | 1.498 |
| 23 | >10.000 |
| 24 | 1.070 |
| 25 | >10.000 |
| 26 | >10.000 |
| 27 | 0.374 |
| 28 | >10.000 |
| 29 | 0.021 |
| 30 | 0.367 |
| 31 | 0.130 |
| 32 | 1.015 |
| 33 | 0.090 |
| 34 | 0.345 |
| 35 | 0.139 |
| 36 | 0.910 |
| 37 | 0.058 |
| 38 | 9.012 |
| 39 | 2.966 |
| 40 | 2.448 |
| 41 | 0.028 |
| 42 | 0.053 |
| 43 | 0.028 |
| 44 | 0.005 |
| 45 | 0.010 |
| 46 | 0.130 |
| 47 | 0.251 |
| 48 | 0.511 |
| 49 | 0.032 |
| 50 | — |
| 51 | — |
| 52 | — |
| 53 | — |
| 54 | — |
| 55 | — |
| 56 | — |
| 57 | — |
| 58 | — |
| 59 | — |
| 60 | — |
| 61 | — |
| 62 | — |
| 63 | — |
| 64 | — |
| 65 | — |
| 66 | 0.006 |
| 67 | 0.027 |
| 68 | 0.003 |
| 69 | 0.001 |
| 70 | 0.004 |
| 71 | 0.003 |
| 72 | 0.012 |
| 73 | 0.027 |
| 74 | 0.514 |
| 75 | 0.355 |
| 76 | 0.175 |
| 77 | 0.042 |
| 78 | 0.022 |
| 79 | 0.316 |
| 80 | 0.064 |
| 81 | 4.29 |
| 82 | 1.231 |
| 83 | 5.876 |
| 84 | 3.437 |
| 85 | 0.0080 |
| 86 | 0.032 |
| 87 | 0.025 |
| 88 | 0.012 |
| 89 | 0.152 |
| 90 | 0.011 |
| 91 | 0.0060 |
| 92 | 0.0004 |
| 93 | 0.0014 |
| 94 | 0.0330 |
| 95 | 0.0530 |
| 96 | 0.044 |
| 97 | 0.011 |
| 98 | 0.144 |
| 99 | 0.037 |
| 100 | 0.017 |
| 101 | 0.026 |
| 102 | 0.240 |
| 103 | >10.000 |
| 104 | 0.309 |
| 105 | 1.020 |
| 106 | 1.041 |
| 107 | 0.133 |
| 108 | 0.024 |
| 109 | >10.000 |
| 110 | 3.555 |
| 111 | 0.007 |
| 112 | 0.097 |
| 113 | 0.029 |
| 114 | 0.116 |
| 115 | 0.003 |
| 116 | 0.009 |
| 117 | 0.013 |
| 118 | 0.011 |
| 119 | 0.014 |
| 120 | 1.057 |
| 121 | 0.027 |
| 122 | 0.514 |
| 123 | 1.831 |
| 124 | 1.630 |
| 125 | 0.102 |
| 126 | 0.550 |
| 127 | 0.035 |
| 128 | 0.095 |
| 129 | 1.632 |
| 130 | 0.042 |
| 131 | 0.067 |
| 132 | 0.180 |
| 133 | 0.573 |
| 134 | 0.844 |
| 135 | 1.091 |
| 136 | 1.145 |
| 137 | 0.018 |
| 138 | 0.458 |
| 139 | 0.106 |
| 140 | 0.015 |
| 141 | 0.043 |
| 142 | 0.522 |
| 143 | 0.066 |
| 144 | 0.051 |
| 145 | 0.697 |
| 146 | 0.496 |
| 147 | 0.165 |
| 148 | 0.287 |
| 149 | 0.476 |
| 150 | 0.061 |
| 151 | 0.376 |
| 152 | — |
| 153 | 0.003 |
| 154 | 0.408 |
| 155 | 0.043 |
| 156 | 0.09 |
| 157 | >10.000 |
| 158 | 3.379 |
| 159 | 0.119 |
| 160 | 0.008 |
| 161 | 0.082 |
| 162 | >10.000 |
| 163 | 0.405 |
| 164 | 0.076 |
| 165 | 0.005 |
| 166 | 0.378 |
| 167 | 0.172 |
| 168 | 0.035 |
| 169 | 0.004 |
| 170 | 2.712 |
| 171 | 0.200 |
| 172 | 0.007 |
| 173 | >10.000 |
| 174 | 0.276 |
| 175 | 0.111 |
| 176 | 0.183 |
| 177 | 0.037 |
| 178 | 2.956 |
| 179 | 0.270 |
| 180 | 1.645 |
| 181 | 6.084 |
| 182 | 0.171 |
| 183 | 4.941 |
| 184 | 0.047 |
| 185 | 0.199 |
| 186 | 9.108 |
| 187 | 0.104 |
| 188 | 0.798 |
| 189 | 0.049 |
| 190 | 0.035 |
| 191 | 0.030 |
| 192 | 0.013 |
| 193 | 0.008 |
| 194 | 0.047 |
| 195 | 0.006 |
| 196 | 0.017 |
| 197 | 0.018 |
| 198 | 0.009 |
| 199 | 0.132 |
| 200 | 1.252 |
| 201 | 0.024 |
| 202 | 0.066 |
| 203 | 0.007 |
| 204 | 0.010 |
| 205 | 0.104 |
| 206 | 0.004 |
| 207 | 0.003 |
| 208 | 0.004 |
| 209 | 0.006 |
| 210 | 0.023 |
| 211 | 0.089 |
| 212 | 0.116 |
| 213 | 0.115 |
| 214 | 0.757 |
| 215 | 0.011 |
| 216 | 0.061 |
| 217 | 0.153 |
| 218 | 0.125 |
| 219 | 0.117 |
| 220 | 0.116 |
| 221 | 0.065 |
| 222 | 0.052 |
| 223 | 0.059 |
| 224 | 0.103 |
| 225 | 0.027 |
| 226 | 0.09 |
| 227 | 0.064 |
| 228 | 0.061 |
| 229 | 0.073 |
| 230 | 0.008 |
| 231 | 0.006 |
| 232 | 0.007 |
| 233 | 0.021 |
| 234 | 0.002 |
| 235 | 0.002 |
| 236 | 0.006 |
| 237 | 0.007 |
| 238 | 0.012 |
| 239 | 0.015 |
| 240 | 0.005 |
| 241 | 0.008 |
| 242 | 0.011 |
| 243 | 0.011 |
| 244 | 0.009 |
| 245 | 0.013 |
| 246 | 0.008 |
| 247 | 0.044 |
| 248 | 0.022 |
| 249 | 0.003 |
| 250 | — |
| 251 | 0.041 |
| 252 | 0.033 |
| 253 | 0.426 |
| 254 | 0.059 |
| 255 | 0.009 |
| 256 | 0.018 |
| 257 | 0.256 |
| 258 | 0.363 |
| 259 | 1.393 |
| 260 | 1.663 |
| 261 | 1.021 |
| 262 | 0.242 |
| 263 | 8.657 |
| 264 | 0.02 |
| 265 | 9.921 |
| 266 | 0.101 |
| 267 | 0.012 |
| 268 | — |
| 269 | 1.53 |
| 270 | 4.816 |
| 271 | >10.000 |
| 272 | 0.055 |
| 273 | 4.234 |
| 274 | 0.132 |

The following are further embodiments of the invention:

### Embodiment 1

A method of modulating BACE-2 activity in a subject, wherein the method comprises administering to the subject a therapeutically effective amount of a compound of formula (I):

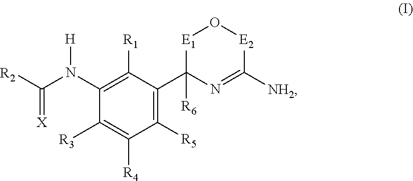

or a pharmaceutically acceptable salt thereof, wherein:
X is O or S;
R₁ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
R₂ is an aryl, heteroaryl or non-aromatic heterocyclyl group G₁, which group G₁ is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3 or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
either
R₄ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl; and
R₅ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ is hydrogen; (C₁₋₈)alkyl; halogen-(C₁₋₈)alkyl; hydroxy-(C₁₋₈)alkyl; (C₁₋₈)alkoxy-(C₁₋₈)alkyl; mercapto-(C₁₋₈)alkyl; (C₁₋₈)alkylthio-(C₁₋₈)alkyl; amino-(C₁₋₈)alkyl; N—(C₁₋₈)alkylamino-(C₁₋₈)alkyl; N,N-di-[(C₁₋₈)alkyl]amino-(C₁₋₈)alkyl with two identical or different (C₁₋₈)alkyl moieties in the N,N-di-[(C₁₋₈)alkyl]amino moiety; (C₂₋₈)alkenyl; or (C₂₋₈)alkynyl;
E₁ is —C(R₇)(R₈)—; or —C(R₇)(R₈)—C(R₉)(R₁₀)—;
E₂ is —C(R₁₁)(R₁₂)—; or —C(R₁₁)(R₁₂)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ and R₁₂ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ and R₁₂, taken together, are oxo or —CH₂—CH₂—; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—.

### Embodiment 2

A method of treating a disorder or disease associated with inhibition of BACE-2 activity, comprising administering to the subject a therapeutically effective amount of a compound of formula (I):

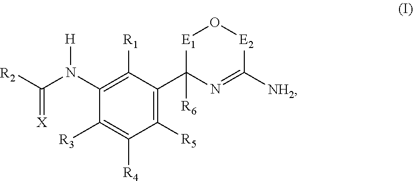

or a pharmaceutically acceptable salt thereof, wherein:
X is O or S;
R₁ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
R₂ is an aryl, heteroaryl or non-aromatic heterocyclyl group G₁, which group G₁ is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3 or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
either
R₄ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl; and
R₅ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[C₁₋₈)alkyl]-, —O—, —S—, —S(═O)— or —S(═O)₂—;
R₆ is hydrogen; (C₁₋₈)alkyl; halogen-(C₁₋₈)alkyl; hydroxy-(C₁₋₈)alkyl; (C₁₋₈)alkoxy-(C₁₋₈)alkyl; mercapto-(C₁₋₈)alkyl; (C₁₋₈)alkylthio-(C₁₋₈)alkyl; amino-(C₁₋₈)alkyl; N—(C₁₋₈)alkylamino-(C₁₋₈)alkyl; N,N-di-[(C₁₋₈)alkyl]amino-(C₁₋₈)alkyl with two identical or different (C₁₋₈)alkyl moieties in the N,N-di-[(C₁₋₈)alkyl]amino moiety; (C₂₋₈)alkenyl; or (C₂₋₈)alkynyl;
E₁ is —C(R₇)(R₈)—; or —C(R₇)(R₈)—C(R₉)(R₁₀)—;
E₂ is —C(R₁₁)(R₁₂)—; or —C(R₁₁)(R₁₂)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ and R₁₂ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ and R₁₂, taken together, are oxo or —CH₂—CH₂—; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
   R₁₃ and R₁₄, taken together, are oxo or —CH₂—CH₂—.

### Embodiment 3

The method according to Embodiment 2 wherein the disorder or disease selected from the group consisting of insulin resistance, impaired glucose tolerance, type 2 diabetes, obesity, hypertension, and diabetic complications.

### Embodiment 4

The method according to Embodiment 3 wherein the disorder or disease selected from the group consisting of impaired glucose tolerance and Type 2 diabetes.

### Embodiment 5

The method according to any one of the preceding Embodiments, wherein X is O.

### Embodiment 6

The method according to any one of the preceding Embodiments, wherein R₁ is hydrogen.

### Embodiment 7

The method according to any one of the preceding Embodiments, wherein R₂ is a heteroaryl or aryl group which is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy and (C₂₋₈)alkynoxy.

### Embodiment 8

The method according to any one of Embodiments 1 to 6, wherein R₂ is a pyridyl or pyrazinyl group which is substituted by 1, 2, 3 or 4 substituents and wherein one of the substituents is located at the para position of the pyridyl or pyrazinyl group relative to the amide linker and wherein the substituents are independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy and (C₂₋₈)alkynoxy.

### Embodiment 9

The method according to any one of Embodiments 1 to 6, wherein R₂ is a pyridyl or pyrazinyl group which is substituted by 1, 2, 3 or 4 substituents and wherein one of the substituents is located at the para position of the pyridyl or pyrazinyl group relative to the amide linker and wherein the substituents are independently selected from the group, consisting of deuterium, cyano, halogen, (C₁₋₆)alkyl, deuterated (C₁₋₆)alkyl, (C₁₋₆)alkoxy, (C₁₋₆)alkoxy-(C₁₋₆)alkoxy, halogen-(C₁₋₆)alkyl and (C₂₋₆)alkynoxy.

### Embodiment 10

The method according to any one of Embodiments 1 to 9, wherein R₃ is hydrogen.

### Embodiment 11

The method according to any one of Embodiments 1 to 10, wherein R₄ is hydrogen or halogen.

### Embodiment 12

The method according to any one of Embodiments 1 to 11, wherein R₅ is hydrogen or halogen.

### Embodiment 13

The method according to any one of Embodiments 1 to 12, wherein R₆ is (C₁₋₃)alkyl; or halogen-(C₁₋₃)alkyl.

### Embodiment 14

The method according to any one of Embodiments 1 to 13, wherein E₁ is CH₂—.

### Embodiment 15

The method according to any one of Embodiments 1 to 14, wherein E₂ is —C(R₁₁)(R₁₂)— and each of R₁₁ and R₁₂ is independently selected from the group, consisting of hydrogen, (C₁₋₃)alkyl and halogen-(C₁₋₃)alkyl.

### Embodiment 16

The method according to any one of Embodiments 1 to 4, wherein the compound is selected from the group consisting of:
Furan-2-carboxylic acid [3-(5-amino-3-methyl-3,6-dihydro-2H-[1,4]oxazin-3-yl)-phenyl]-amide;
5-Bromo-pyridine-2-carboxylic acid [3-(5-amino-3-methyl-3,6-dihydro-2H-[1,4]oxazin-3-yl)-phenyl]-amide;
5-Bromo-pyridine-2-carboxylic acid [3-((R)-5-amino-3-methyl-3,6-di [symbol] hydro-2H-[1,4]oxazin-3-yl)-phenyl]-amide;
5-Bromo-pyridine-2-carboxylic acid [3-((S)-5-amino-3-methyl-3,6-di [symbol] hydro-2H-[1,4]oxazin-3-yl)-phenyl]-amide;
2-Methyl-oxazole-4-carboxylic acid [3-(5-amino-3-methyl-3,6-dihydro-2H-[1,4]oxazin-3-yl)-phenyl]-amide;
5-Methyl-pyrazine-2-carboxylic acid [3-(5-amino-3-methyl-3,6-dihydro-2H-[1,4]oxazin-3-yl)-phenyl]-amide;
5-Bromo-pyrimidine-2-carboxylic acid [3-(5-amino-3-methyl-3,6-dihydro-2H-[1,4]oxazin-3-yl)-phenyl]-amide;
Imidazo[1,2-a]pyridine-2-carboxylic acid [3-(5-amino-3-methyl-3,6-dihydro-2H-[1,4]oxazin-3-yl)-phenyl]-amide;
3-Fluoro-pyridine-2-carboxylic acid [3-(5-amino-3-methyl-3,6-dihydro-2H-[1,4]oxazin-3-yl)-phenyl]-amide;
2,5-Dimethyl-oxazole-4-carboxylic acid [3-(5-amino-3-methyl-3,6-dihydro-2H-[1,4]oxazin-3-yl)-phenyl]-amide;
2-Methyl-thiazole-4-carboxylic acid [3-(5-amino-3-methyl-3,6-dihydro-2H-[1,4]oxazin-3-yl)-phenyl]-amide;
6-Hydroxy-pyridazine-3-carboxylic acid [3-(5-amino-3-methyl-3,6-dihydro-2H-[1,4]oxazin-3-yl)-phenyl]-amide;
Pyridine-2-carboxylic acid [3-(5-amino-3-methyl-3,6-dihydro-2H-[1,4]oxazin-3-yl)-phenyl]-amide;
5-Cyano-pyridine-2-carboxylic acid [3-(5-amino-3-methyl-3,6-dihydro-2H-[1,4]oxazin-3-yl)-phenyl]-amide;
5-(3-Trifluoromethyl-pyrazol-1-yl)-pyrazine-2-carboxylic acid [3-(5-amino-3-methyl-3,6-dihydro-2H-[1,4]oxazin-3-yl)-phenyl]-amide;
5-(3-Methyl-pyrazol-1-yl)-pyrazine-2-carboxylic acid [3-(5-amino-3-methyl-3,6-dihydro-2H-[1,4]oxazin-3-yl)-phenyl]-amide;
3-Chloro-5-trifluoromethyl-pyridine-2-carboxylic acid [3-(5-amino-3-methyl-3,6-dihydro-2H-[1,4]oxazin-3-yl)-phenyl]-amide;
5-Methoxy-pyrazine-2-carboxylic acid [3-(5-amino-3-methyl-3,6-dihydro-2H-[1,4]oxazin-3-yl)-phenyl]-amide;
5-Hydroxy-pyrazine-2-carboxylic acid [3-(5-amino-3-methyl-3,6-dihydro-2H-[1,4]oxazin-3-yl)-phenyl]-amide;
4-Bromo-furan-2-carboxylic acid [3-(5-amino-3-methyl-3,6-dihydro-2H-[1,4]oxazin-3-yl)-phenyl]-amide;
5-Chloro-pyridine-2-carboxylic acid [3-(5-amino-3-methyl-3,6-dihydro-2H-[1,4]oxazin-3-yl)-phenyl]-amide;
5-Trifluoromethyl-pyridine-2-carboxylic acid [3-(5-amino-3-methyl-3,6-dihydro-2H-[1,4]oxazin-3-yl)-phenyl]-amide;
5-Trifluoromethyl-furan-3-carboxylic acid [3-(5-amino-3-methyl-3,6-dihydro-2H-[1,4]oxazin-3-yl)-phenyl]-amide;
N-[3-(5-Amino-3-methyl-3,6-dihydro-2H-[1,4]oxazin-3-yl)-phenyl]-4-bromo-benzamide;
5-Methyl-pyridine-2-carboxylic acid [3-(5-amino-3-methyl-3,6-dihydro-2H-[1,4]oxazin-3-yl)-phenyl]-amide;
N-[3-(5-Amino-3-methyl-3,6-dihydro-2H-[1,4]oxazin-3-yl)-phenyl]-nicotinamide;
5-Bromo-3-methyl-pyridine-2-carboxylic acid [3-((R)-5-amino-3-methyl-3,6-dihydro-2H-[1,4]oxazin-3-yl)-phenyl]-amide;
5-Methoxy-pyridine-2-carboxylic acid [3-((R)-5-amino-3-methyl-3,6-dihydro-2H-[1,4]oxazin-3-yl)-phenyl]-amide;
5-Bromo-pyridine-2-carboxylic acid [3-(5-amino-3-fluoromethyl-3,6-dihydro-2H-[1,4]oxazin-3-yl)-phenyl]-amide;
5-Bromo-pyridine-2-carboxylic acid [3-(5-amino-3-fluoromethyl-3,6-dihydro-2H-[1,4]oxazin-3-yl)-5-chloro-phenyl]-amide;
5-Bromo-pyridine-2-carboxylic acid [3-((R)-5-amino-3-fluoromethyl-3,6-dihydro-2H-[1,4]oxazin-3-yl)-phenyl]-amide;
5-Bromo-pyridine-2-carboxylic acid [3-(5-amino-3-difluoromethyl-3,6-dihydro-2H-[1,4]oxazin-3-yl)-phenyl]-amide;
5-Chloro-pyridine-2-carboxylic acid [3-(5-amino-3-difluoromethyl-3,6-dihydro-2H-[1,4]oxazin-3-yl)-phenyl]-amide;
5-Cyano-pyridine-2-carboxylic acid [3-(5-amino-3-difluoromethyl-3,6-dihydro-2H-[1,4]oxazin-3-yl)-phenyl]-amide;
5-Bromo-3-methyl-pyridine-2-carboxylic acid [3-(5-amino-3-difluoromethyl-3,6-dihydro-2H-[1,4]oxazin-3-yl)-phenyl]-amide;
5-Methyl-pyrazine-2-carboxylic acid [3-(5-amino-3-difluoromethyl-3,6-dihydro-2H-[1,4]oxazin-3-yl)-phenyl]-amide;
5-Trifluoromethyl-pyridine-2-carboxylic acid [3-(5-amino-3-difluoromethyl-3,6-dihydro-2H-[1,4]oxazin-3-yl)-phenyl]-amide;
3-Chloro-5-trifluoromethyl-pyridine-2-carboxylic acid [3-(5-amino-3-difluoromethyl-3,6-dihydro-2H-[1,4]oxazin-3-yl)-phenyl]-amide;
5-Chloro-3-methyl-pyridine-2-carboxylic acid [3-(5-amino-3-difluoromethyl-3,6-dihydro-2H-[1,4]oxazin-3-yl)-phenyl]-amide;
5-Bromo-pyridine-2-carboxylic acid [3-(5-amino-3-difluoromethyl-3,6-dihydro-2H-[1,4]oxazin-3-yl)-4-fluoro-phenyl]-amide;
5-Chloro-pyridine-2-carboxylic acid [3-(5-amino-3-difluoromethyl-3,6-dihydro-2H-[1,4]oxazin-3-yl)-4-fluoro-phenyl]-amide;
5-Chloro-3-methyl-pyridine-2-carboxylic acid [3-((R)-5-amino-3-difluoromethyl-3,6-dihydro-2H-[1,4]oxazin-3-yl)-4-fluoro-phenyl]-amide;
5-Bromo-3-methyl-pyridine-2-carboxylic acid [3-((R)-5-amino-3-difluoromethyl-3,6-dihydro-2H-[1,4]oxazin-3-yl)-4-fluoro-phenyl]-amide;
3-Chloro-5-trifluoromethyl-pyridine-2-carboxylic acid [3-((R)-5-amino-3-difluoromethyl-3,6-dihydro-2H-[1,4]oxazin-3-yl)-4-fluoro-phenyl]-amide;
5-Trifluoromethyl-pyridine-2-carboxylic acid [3-((R)-5-amino-3-difluoromethyl-3,6-dihydro-2H-[1,4]oxazin-3-yl)-4-fluoro-phenyl]-amide;
5-Methyl-pyrazine-2-carboxylic acid [3-((R)-5-amino-3-difluoromethyl-3,6-dihydro-2H-[1,4]oxazin-3-yl)-4-fluoro-phenyl]-amide;
5-Methoxy-pyridine-2-carboxylic acid [3-((R)-5-amino-3-difluoromethyl-3,6-dihydro-2H-[1,4]oxazin-3-yl)-4-fluoro-phenyl]-amide;
5-Chloro-pyridine-2-carboxylic acid [3-(5-amino-3-trifluoromethyl-3,6-dihydro-2H-[1,4]oxazin-3-yl)-4-fluoro-phenyl]-amide;
5-Bromo-pyrimidine-2-carboxylic acid [3-((R)-5-amino-3-methyl-3,6-dihydro-2H-[1,4]oxazin-3-yl)-5-bromo-phenyl]-amide;
5-Bromo-pyridine-2-carboxylic acid [3-((R)-5-amino-3-methyl-3,6-dihydro-2H-[1,4]oxazin-3-yl)-5-bromo-phenyl]-amide;
5-Cyano-pyridine-2-carboxylic acid [3-((R)-5-amino-3-methyl-3,6-dihydro-2H-[1,4]oxazin-3-yl)-5-bromo-phenyl]-amide;
5-Trifluoromethyl-pyridine-2-carboxylic acid [3-((R)-5-amino-3-methyl-3,6-dihydro-2H-[1,4]oxazin-3-yl)-5-bromo-phenyl]-amide;
5-Bromo-3-methyl-pyridine-2-carboxylic acid [3-((R)-5-amino-3-methyl-3,6-dihydro-2H-[1,4]oxazin-3-yl)-5-bromo-phenyl]-amide;
5-Chloro-pyridine-2-carboxylic acid [3-((R)-5-amino-3-methyl-3,6-dihydro-2H-[1,4]oxazin-3-yl)-5-bromo-phenyl]-amide;
5-Methyl-pyrazine-2-carboxylic acid [3-((R)-5-amino-3-methyl-3,6-dihydro-2H-[1,4]oxazin-3-yl)-5-bromo-phenyl]-amide;
2,5-Dimethyl-oxazole-4-carboxylic acid [3-((R)-5-amino-3-methyl-3,6-dihydro-2H-[1,4]oxazin-3-yl)-5-bromo-phenyl]-amide;
2-Methyl-oxazole-4-carboxylic acid [3-((R)-5-amino-3-methyl-3,6-dihydro-2H-[1,4]oxazin-3-yl)-5-bromo-phenyl]-amide;
5-Bromo-pyridine-2-carboxylic acid [3-(5-amino-3-methyl-3,6-dihydro-2H-[1,4]oxazin-3-yl)-4-fluoro-phenyl]-amide;
5-Methyl-pyrazine-2-carboxylic acid [3-(5-amino-3-methyl-3,6-dihydro-2H-[1,4]oxazin-3-yl)-4-fluoro-phenyl]-amide;
5-Cyano-pyridine-2-carboxylic acid [3-(5-amino-3-methyl-3,6-dihydro-2H-[1,4]oxazin-3-yl)-4-fluoro-phenyl]-amide;
5-Chloro-pyridine-2-carboxylic acid [3-(5-amino-3-methyl-3,6-dihydro-2H-[1,4]oxazin-3-yl)-4-fluoro-phenyl]-amide;
5-Bromo-3-methyl-pyridine-2-carboxylic acid [3-(5-amino-3-methyl-3,6-dihydro-2H-[1,4]oxazin-3-yl)-4-fluoro-phenyl]-amide;
Pyridine-2,5-dicarboxylic acid 5-amide 2-{[3-(5-amino-3-methyl-3,6-dihydro-2H-[1,4]oxazin-3-yl)-4-fluoro-phenyl]-amide};
5-Bromo-pyridine-2-carboxylic acid [3-((3R,6R)-5-amino-3,6-dimethyl-6-trifluoromethyl-3,6-dihydro-2H-[1,4]oxazin-3-yl)-phenyl]-amide;
5-Methoxy-pyridine-2-carboxylic acid [3-((3R,6R)-5-amino-3,6-dimethyl-6-trifluoromethyl-3,6-dihydro-2H-[1,4]oxazin-3-yl)-phenyl]-amide;
5-Bromo-pyridine-2-carboxylic acid [3-((3R,6R)-5-amino-3,6-di-methyl-6-trifluoromethyl-3,6-di-hydro-2H-[1,4]oxazin-3-yl)-4-fluoro-phenyl]-amide;
5-Methoxy-pyridine-2-carboxylic acid [3-((3R,6R)-5-amino-3,6-di-methyl-6-trifluoro [symbol] methyl-3,6-di-hydro-2H-[1,4]oxazin-3-yl)-4-fluoro-phenyl]-amide;
5-Cyano-pyrimidine-2-carboxylic acid [3-((3R,6R)-5-amino-3,6-di-methyl-6-trifluoromethyl-3,6-di-hydro-2H-[1,4]oxazin-3-yl)-4-fluoro-phenyl]-amide;
5-Bromo-pyrimidine-2-carboxylic acid [3-((3R,6R)-5-amino-3,6-dimethyl-6-trifluoro-methyl-3,6-dihydro-2H-[1,4]oxazin-3-yl)-4-fluoro-phenyl]-amide;
5-Cyano-3-methyl-pyridine-2-carboxylic acid [3-((3R,6R)-5-amino-3,6-dimethyl-6-trifluoro-methyl-3,6-dihydro-2H-[1,4]oxazin-3-yl)-4-fluoro-phenyl]-amide;
5-Cyano-pyridine-2-carboxylic acid [3-((3R,6R)-5-amino-3,6-dimethyl-6-trifluoro-methyl-3,6-dihydro-2H-[1,4]oxazin-3-yl)-4-fluoro-phenyl]-amide;
5-(2-Methoxy-ethoxy)-pyrazine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
Cyano-pyridine-2-carboxylic acid [3-((3R,6R)-5-amino-3-difluoromethyl-6-methyl-3,6-dihydro-2H-[1,4]oxazin-3-yl)-4-fluoro-phenyl]-amide;
Cyano-pyridine-2-carboxylic acid [3-((3R,6S)-5-amino-3-difluoromethyl-6-methyl-3,6-dihydro-2H-[1,4]oxazin-3-yl)-4-fluoro-phenyl]-amide;
5-Bromo-pyridine-2-carboxylic acid[3-((3R,6R)-5-amino-3-difluoromethyl-6-methyl-3,6-dihydro-2H-[1,4]oxazin-3-yl)-4-fluoro-phenyl]-amide;
5-Bromo-pyridine-2-carboxylic acid[3-((3S,6R)-5-amino-3-difluoromethyl-6-methyl-3,6-dihydro-2H-[1,4]oxazin-3-yl)-4-fluoro-phenyl]-amide;
5-Cyano-pyridine-2-carboxylic acid[3-(5-amino-3-difluoromethyl-6,6-dimethyl-3,6-dihydro-2H-[1,4]oxazin-3-yl)-4-fluoro-phenyl]-amide;
5-Bromo-pyridine-2-carboxylic acid [3-(5-amino-3-difluoromethyl-6,6-dimethyl-3,6-dihydro-2H-[1,4]oxazin-3-yl)-4-fluoro-phenyl]-amide;
5-Bromo-pyridine-2-carboxylic acid [5-(5-amino-3-difluoromethyl-3,6-dihydro-2H-[1,4]oxazin-3-yl)-2,4-difluoro-phenyl]-amide;
5-Cyano-pyridine-2-carboxylic acid [5-(5-amino-3-difluoromethyl-3,6-dihydro-2H-[1,4]oxazin-3-yl)-2,4-difluoro-phenyl]-amide;
5-Bromo-3-methoxy-pyridine-2-carboxylic acid [5-(5-amino-3-difluoromethyl-3,6-dihydro-2H-[1,4]oxazin-3-yl)-2,4-difluoro-phenyl]-amide;
5-Bromo-3-hydroxy-pyridine-2-carboxylic acid [5-(5-amino-3-difluoromethyl-3,6-dihydro-2H-[1,4]oxazin-3-yl)-2,4-difluoro-phenyl]-amide;
5-Chloro-pyridine-2-carboxylic acid [3-((R)-5-amino-3-fluoromethyl-3,6-dihydro-2H-[1,4]oxazin-3-yl)-4-fluoro-phenyl]-amide;
5-Bromo-pyridine-2-carboxylic acid [3-(5-amino-3-fluoromethyl-3,6-dihydro-2H-[1,4]oxazin-3-yl)-4-fluoro-phenyl]-amide;
5-Chloro-pyridine-2-carboxylic acid [3-(5-amino-3-fluoromethyl-3,6-dihydro-2H-[1,4]oxazin-3-yl)-4-fluoro-phenyl]-amide;
3,5-Dichloro-pyridine-2-carboxylic acid [3-(5-amino-3-fluoromethyl-3,6-dihydro-2H-[1,4]oxazin-3-yl)-4-fluoro-phenyl]-amide;
5-Cyano-pyridine-2-carboxylic acid [3-(5-amino-3-fluoromethyl-3,6-dihydro-2H-[1,4]oxazin-3-yl)-4-fluoro-phenyl]-amide;
5-Bromo-3-methyl-pyridine-2-carboxylic acid [3-(5-amino-3-fluoromethyl-3,6-dihydro-2H-[1,4]oxazin-3-yl)-4-fluoro-phenyl]-amide;
5-Bromo-3-methyl-pyridine-2-carboxylic acid [3-((R)-5-amino-3-fluoromethyl-3,6-dihydro-2H-[1,4]oxazin-3-yl)-4-fluoro-phenyl]-amide;
5-Chloro-3-methyl-pyridine-2-carboxylic acid [3-((R)-5-amino-3-fluoromethyl-3,6-dihydro-2H-[1,4]oxazin-3-yl)-4-fluoro-phenyl]-amide;
5-Chloro-4,6-dideutero-3-trideuteromethyl-pyridine-2-carboxylic acid [3-((R)-5-amino-3-fluoromethyl-3,6-dihydro-2H-[1,4]oxazin-3-yl)-4-fluoro-phenyl]-amide;
5-Bromo-3-hydroxy-pyridine-2-carboxylic acid [3-((R)-5-amino-3-fluoromethyl-3,6-dihydro-2H-[1,4]oxazin-3-yl)-4-fluoro-phenyl]-amide;
5-Bromo-3-methoxy-pyridine-2-carboxylic acid [3-((R)-5-amino-3-fluoromethyl-3,6-dihydro-2H-[1,4]oxazin-3-yl)-4-fluoro-phenyl]-amide;
5-Bromo-pyridine-2-carboxylic acid[3-((S)-3-amino-5-difluoromethyl-2,5,6,7-tetrahydro-[1,4]oxazepin-5-yl)-4-fluoro-phenyl]-amide;
5-Bromo-pyridine-2-carboxylic acid[3-((S)-3-amino-5-difluoromethyl-2,5,6,7-tetrahydro-[1,4]oxazepin-5-yl)-4-fluoro-phenyl]-amide;
5-Cyano-pyridine-2-carboxylic acid [3-(3-amino-5-trifluoromethyl-2,5,6,7-tetrahydro-[1,4]oxazepin-5-yl)-4-fluoro-phenyl]-amide;
5-Bromo-pyridine-2-carboxylic acid [3-(3-amino-5-trifluoromethyl-2,5,6,7-tetrahydro-[1,4]oxazepin-5-yl)-4-fluoro-phenyl]-amide;
N-(3-(3-amino-6,6-difluoro-5-methyl-2,5,6,7-tetrahydro-1,4-oxazepin-5-yl)-4-fluorophenyl)-5-chloropicolinamide;
5-Bromo-pyridine-2-carboxylic acid [3-(3-amino-6,6-difluoro-5-methyl-2,5,6,7-tetrahydro-[1,4]oxazepin-5-yl)-4-fluoro-phenyl]-amide;
4-Bromo-furan-2-carboxylic acid [3-(5-amino-3-difluoromethyl-3,6-dihydro-2H-[1,4]oxazin-3-yl)-phenyl]-amide;
6-Hydroxy-pyridazine-3-carboxylic acid [3-(5-amino-3-difluoromethyl-3,6-dihydro-2H-[1,4]oxazin-3-yl)-phenyl]-amide;
2-Methyl-oxazole-4-carboxylic acid [3-(5-amino-3-difluoromethyl-3,6-dihydro-2H-[1,4]oxazin-3-yl)-phenyl]-amide;
2-Ethyl-oxazole-4-carboxylic acid [3-(5-amino-3-difluoromethyl-3,6-dihydro-2H-[1,4]oxazin-3-yl)-phenyl]-amide;
2,5-Dimethyl-oxazole-4-carboxylic acid [3-(5-amino-3-difluoromethyl-3,6-dihydro-2H-[1,4]oxazin-3-yl)-phenyl]-amide;
5-Bromo-3-methoxy-pyridine-2-carboxylic acid [3-(5-amino-3-difluoromethyl-3,6-dihydro-2H-[1,4]oxazin-3-yl)-phenyl]-amide;
5-Bromo-3-hydroxy-pyridine-2-carboxylic acid [3-(5-amino-3-difluoromethyl-3,6-dihydro-2H-[1,4]oxazin-3-yl)-phenyl]-amide;
5-(2-Methoxy-ethoxy)-pyridine-2-carboxylic acid [3-(5-amino-3-difluoromethyl-3,6-dihydro-2H-[1,4]oxazin-3-yl)-phenyl]-amide;
5-Difluoromethyl-pyrazine-2-carboxylic acid [3-(5-amino-3-difluoromethyl-3,6-dihydro-2H-[1,4]oxazin-3-yl)-phenyl]-amide;
3,5-Dichloro-pyridine-2-carboxylic acid [3-((R)-5-amino-3-difluoromethyl-3,6-dihydro-2H-[1,4]oxazin-3-yl)-4-fluoro-phenyl]-amide;
5-Cyano-pyridine-2-carboxylic acid [3-((R)-5-amino-3-difluoromethyl-3,6-dihydro-2H-[1,4]oxazin-3-yl)-4-fluoro-phenyl]-amide;
3,5-Difluoro-pyridine-2-carboxylic acid [3-((R)-5-amino-3-difluoromethyl-3,6-dihydro-2H-[1,4]oxazin-3-yl)-4-fluoro-phenyl]-amide;
5-Bromo-3-methyl-benzofuran-2-carboxylic acid [3-((R)-5-amino-3-difluoromethyl-3,6-dihydro-2H-[1,4]oxazin-3-yl)-4-fluoro-phenyl]-amide;
5-Chloro-4,6-dideutero-3-trideuteromethyl-pyridine-2-carboxylic acid [3-((R)-5-amino-3-difluoromethyl-3,6-dihydro-2H-[1,4]oxazin-3-yl)-4-fluoro-phenyl]-amide;
5-Bromo-4,6-dideutero-3-trideuteromethyl-pyridine-2-carboxylic acid [3-((R)-5-amino-3-difluoromethyl-3,6-dihydro-2H-[1,4]oxazin-3-yl)-4-fluoro-phenyl]-amide;
5-Bromo-3-chloro-pyridine-2-carboxylic acid [3-((R)-5-amino-3-difluoromethyl-3,6-dihydro-2H-[1,4]oxazin-3-yl)-4-fluoro-phenyl]-amide;
5-Bromo-pyridine-2-carboxylic acid [3-((R)-5-amino-3-difluoromethyl-3,6-dihydro-2H-[1,4]oxazin-3-yl)-4-fluoro-phenyl]-amide;
5-Bromo-3-hydroxy-pyridine-2-carboxylic acid [3-((R)-5-amino-3-difluoromethyl-3,6-dihydro-2H-[1,4]oxazin-3-yl)-4-fluoro-phenyl]-amide;
5-Ethoxy-pyridine-2-carboxylic acid [3-((R)-5-amino-3-difluoromethyl-3,6-dihydro-2H-[1,4]oxazin-3-yl)-4-fluoro-phenyl]-amide;
5-Chloro-pyrimidine-2-carboxylic acid [3-((R)-5-amino-3-difluoromethyl-3,6-dihydro-2H-[1,4]oxazin-3-yl)-4-fluoro-phenyl]-amide;
5-Difluoromethyl-pyrazine-2-carboxylic acid [3-((R)-5-amino-3-difluoromethyl-3,6-dihydro-2H-[1,4]oxazin-3-yl)-4-fluoro-phenyl]-amide;
5-(2,2-Difluoro-ethoxy)-pyrazine-2-carboxylic acid [3-((R)-5-amino-3-difluoromethyl-3,6-dihydro-2H-[1,4]oxazin-3-yl)-4-fluoro-phenyl]-amide;
5-(2,2,2-Trifluoro-ethoxy)-pyrazine-2-carboxylic acid [3-((R)-5-amino-3-difluoromethyl-3,6-dihydro-2H-[1,4]oxazin-3-yl)-4-fluoro-phenyl]-amide;
4-Bromo-furan-2-carboxylic acid [3-((R)-5-amino-3-difluoromethyl-3,6-dihydro-2H-[1,4]oxazin-3-yl)-4-fluoro-phenyl]-amide;
Pyrazolo[1,5-a]pyridine-2-carboxylic acid [3-((R)-5-amino-3 difluoromethyl-3,6-dihydro-2H [1,4]oxazin-3-yl)-4-fluoro-phenyl]-amide;
2-Methyl-oxazole-4-carboxylic acid [3-((R)-5-amino-3-difluoromethyl-3,6-dihydro-2H-[1,4]oxazin-3-yl)-4-fluoro-phenyl]-amide;
2,5-Dimethyl-oxazole-4-carboxylic acid [3-((R)-5-amino-3-difluoromethyl-3,6-dihydro-2H-[1,4]oxazin-3-yl)-4-fluoro-phenyl]-amide;
Imidazo[1,2-a]pyridine-2-carboxylic acid [3-((R)-5-amino-3-difluoromethyl-3,6-dihydro-2H-[1,4]oxazin-3-yl)-4-fluoro-phenyl]-amide;
5-Bromo-3-methoxy-pyridine-2-carboxylic acid [3-((R)-5-amino-3-difluoromethyl-3,6-dihydro-2H-[1,4]oxazin-3-yl)-4-fluoro-phenyl]-amide;
2-Ethyl-oxazole-4-carboxylic acid [3-((R)-5-amino-3-difluoromethyl-3,6-dihydro-2H-[1,4]oxazin-3-yl)-4-fluoro-phenyl]-amide;
5-Difluoromethyl-pyridine-2-carboxylic acid [3-((R)-5-amino-3-difluoromethyl-3,6-dihydro-2H-[1,4]oxazin-3-yl)-4-fluoro-phenyl]-amide;
1-Methyl-1H-imidazole-2 carboxylic acid [3-((R)-5-amino-3 difluoromethyl-3,6-dihydro-2H [1,4]oxazin-3-yl)-4-fluoro-phenyl]-amide;
6-Hydroxy-pyridazine-3-carboxylic acid [3-((R)-5-amino-3-difluoromethyl-3,6-dihydro-2H-[1,4]oxazin-3-yl)-4-fluoro-phenyl]-amide;
5-(2-Methoxy-ethoxy)-pyrazine-2-carboxylic acid [3-((R)-5-amino-3-difluoromethyl-3,6-dihydro-2H-[1,4]oxazin-3-yl)-4-fluoro-phenyl]-amide;
5-(2-Fluoro-ethoxy)-pyrazine-2-carboxylic acid [3-((R)-5-amino-3-difluoromethyl-3,6-dihydro-2H-[1,4]oxazin-3-yl)-4-fluoro-phenyl]-amide;
5-Chloro-pyridine-2-carboxylic acid [3-((R)-5-amino-3-difluoromethyl-3,6-dihydro-2H-[1,4]oxazin-3-yl)-4-fluoro-phenyl]-amide;
5-Difluoromethoxy-pyridine-2-carboxylic acid [3-((R)-5-amino-3-difluoromethyl-3,6-dihydro-2H-[1,4]oxazin-3-yl)-4-fluoro-phenyl]-amide;
5-Fluoromethoxy-pyridine-2-carboxylic acid [3-((R)-5-amino-3-difluoromethyl-3,6-dihydro-2H-[1,4]oxazin-3-yl)-4-fluoro-phenyl]-amide;
5-Chloro-3-fluoro-pyridine-2-carboxylic acid [3-((R)-5-amino-3-difluoromethyl-3,6-dihydro-2H-[1,4]oxazin-3-yl)-4-fluoro-phenyl]-amide;
5-Chloro-pyrazine-2-carboxylic acid [3-((R)-5-amino-3-difluoromethyl-3,6-dihydro-2H-[1,4]oxazin-3-yl)-4-fluoro-phenyl]-amide;
5-Methyl-1H-pyrazole-3-carboxylic acid [3-((R)-5-amino-3-difluoromethyl-3,6-dihydro-2H-[1,4]oxazin-3-yl)-4-fluoro-phenyl]-amide;
5-Methyl-pyridine-2-carboxylic acid [3-((R)-5-amino-3-difluoromethyl-3,6-dihydro-2H-[1,4]oxazin-3-yl)-4-fluoro-phenyl]-amide;
5-Cyano-3-methyl-pyridine-2-carboxylic acid [3-((R)-5-amino-3-difluoromethyl-3,6-dihydro-2H-[1,4]oxazin-3-yl)-4-fluoro-phenyl]-amide;
5-Hydroxy-pyrazine-2-carboxylic acid [3-((R)-5-amino-3-difluoromethyl-3,6-dihydro-2H-[1,4]oxazin-3-yl)-4-fluoro-phenyl]-amide;
5-Methoxy-pyrazine-2-carboxylic acid [3-((R)-5-amino-3-difluoromethyl-3,6-dihydro-2H-[1,4]oxazin-3-yl)-4-fluoro-phenyl]-amide;
5-Cyano-4,6-dideutero-3-trideuteromethyl-pyridine-2-carboxylic acid [3-((R)-5-amino-3-difluoromethyl-3,6-dihydro-2H-[1,4]oxazin-3-yl)-4-fluoro-phenyl]-amide;
2-Methyl-thiazole-4-carboxylic acid [3-((R)-5-amino-3-difluoromethyl-3,6-dihydro-2H-[1,4]oxazin-3-yl)-4-fluoro-phenyl]-amide;
5-Methyl-thiazole-2-carboxylic acid [3-((R)-5-amino-3-difluoromethyl-3,6-dihydro-2H-[1,4]oxazin-3-yl)-4-fluoro-phenyl]-amide;
1-Methyl-1H-pyrazole-3-carboxylic acid [3-((R)-5-amino-3-difluoromethyl-3,6-dihydro-2H-[1,4]oxazin-3-yl)-4-fluoro-phenyl]-amide;
1-Methyl-4-nitro-1H-pyrazole-3-carboxylic acid [3-((R)-5-amino-3-difluoromethyl-3,6-dihydro-2H-[1,4]oxazin-3-yl)-4-fluoro-phenyl]-amide;
3-Amino-5-chloro-pyridine-2-carboxylic acid [3-((3R,6R)-5-amino-3,6-dimethyl-6-trifluoro-methyl-3,6-dihydro-2H-[1,4]oxazin-3-yl)-4-fluoro-phenyl]-amide;
3-Chloro-5-trifluoromethyl-pyridine-2-carboxylic acid [3-(3-amino-6,6-difluoro-5-methyl-2,5,6,7-tetrahydro-[1,4]oxazepin-5-yl)-4-fluoro-phenyl]-amide;
5-Bromo-3-methoxy-pyridine-2-carboxylic acid [3-(3-amino-6,6 difluoro-5-methyl-2,5,6,7 tetrahydro-[1,4]oxazepin-5-yl)-4 fluoro-phenyl]-amide;
5-Cyano-pyridine-2-carboxylic acid [3-(3-amino-6,6-difluoro-5 methyl-2,5,6,7-tetrahydro[1,4]oxazepin-5-yl)-4 fluoro-phenyl]-amide;
7H-Pyrrolo[2,3-d]pyrimidine-6-carboxylic acid [3-((R)-5-amino-3-difluoromethyl-3,6-dihydro-2H-[1,4]oxazin-3-yl)-4-fluoro-phenyl]-amide;
3-Amino-5-(2-methoxy-ethoxy)-pyrazine-2-carboxylic acid [3-((R)-5-amino-3-difluoromethyl-3,6-dihydro-2H-[1,4]oxazin-3-yl)-4-fluoro-phenyl]-amide;
((R)-5-Difluoromethyl-5-{5-[(5-ethyl-pyridine-2-carbonyl)-amino]-2-fluoro-phenyl}-5,6-dihydro-2H-[1,4]oxazin-3-yl)-carbamic acid tert-butyl ester;
3-Amino-5-chloro-pyridine-2-carboxylic acid [3-((R)-5-amino-3-difluoromethyl-3,6-dihydro-2H-[1,4]oxazin-3-yl)-4-fluoro-phenyl]-amide;
3-Chloro-5-methoxy-pyridine-2-carboxylic acid [3-((R)-5-amino-3-difluoromethyl-3,6-dihydro-2H-[1,4]oxazin-3-yl)-4-fluoro-phenyl]-amide;
6-Oxo-1,6-dihydro-pyridine-3-carboxylic acid [3-((R)-5-amino-3-difluoromethyl-3,6-dihydro-2H-[1,4]oxazin-3-yl)-4-fluoro-phenyl]-amide;
Pyrrolo[1,2-c]pyrimidine-3-carboxylic acid [3-((R)-5-amino-3-difluoromethyl-3,6-dihydro-2H-[1,4]oxazin-3-yl)-4-fluoro-phenyl]-amide;
5-But-2-ynyloxy-pyrazine-2-carboxylic acid [3-((R)-5-amino-3-difluoromethyl-3,6-dihydro-2H-[1,4]oxazin-3-yl)-4-fluoro-phenyl]-amide;
3-Amino-5-bromo-pyridine-2-carboxylic acid [3-((R)-5-amino-3-difluoromethyl-3,6-dihydro-2H-[1,4]oxazin-3-yl)-4-fluoro-phenyl]-amide;
1-Ethyl-1H-imidazole-2-carboxylic acid [3-((R)-5-amino-3-difluoromethyl-3,6-dihydro-2H-[1,4]oxazin-3-yl)-4-fluoro-phenyl]-amide;
5-Prop-2-ynyloxy-pyrazine-2-carboxylic acid [3-((R)-5-amino-3-difluoromethyl-3,6-dihydro-2H-[1,4]oxazin-3-yl)-4-fluoro-phenyl]-amide;
5-Amino-2-methyl-oxazole-4-carboxylic acid [3-((R)-5-amino-3-difluoromethyl-3,6-dihydro-2H-[1,4]oxazin-3-yl)-4-fluoro-phenyl]-amide;
5-Chloro-3-hydroxy-pyridine-2-carboxylic acid [3-((R)-5-amino-3-difluoromethyl-3,6-dihydro-2H-[1,4]oxazin-3-yl)-4-fluoro-phenyl]-amide;
5-Isopropoxy-pyrazine-2-carboxylic acid [3-((R)-5-amino-3-difluoromethyl-3,6-dihydro-2H-[1,4]oxazin-3-yl)-4-fluoro-phenyl]-amide;
5-Ethoxy-pyrazine-2-carboxylic acid [3-((R)-5-amino-3-difluoromethyl-3,6-dihydro-2H-[1,4]oxazin-3-yl)-4-fluoro-phenyl]-amide;
5-Dimethylaminomethyl-3-methyl-benzofuran-2-carboxylic acid [3-((R)-5-amino-3-difluoromethyl-3,6-dihydro-2H-[1,4]oxazin-3-yl)-4-fluoro-phenyl]-amide;
1,5-Dimethyl-1H-[1,2,3]triazole-4-carboxylic acid [3-((R)-5-amino-3-difluoromethyl-3,6-dihydro-2H-[1,4]oxazin-3-yl)-4-fluoro-phenyl]-amide;
5-Methoxy-3-methyl-pyrazine-2-carboxylic acid [3-((R)-5-amino-3-difluoromethyl-3,6-dihydro-2H-[1,4]oxazin-3-yl)-4-fluoro-phenyl]-amide;
3-Amino-5-methoxy-pyrazine-2-carboxylic acid [3-((R)-5-amino-3-difluoromethyl-3,6-dihydro-2H-[1,4]oxazin-3-yl)-4-fluoro-phenyl]-amide;
5-Difluoromethoxy-3-methyl-pyridine-2-carboxylic acid [3-((R)-5-amino-3-difluoromethyl-3,6-dihydro-2H-[1,4]oxazin-3-yl)-4-fluoro-phenyl]-amide;
5-Fluoromethoxy-3-methyl-pyridine-2-carboxylic acid [3-((R)-5-amino-3-difluoromethyl-3,6-dihydro-2H-[1,4]oxazin-3-yl)-4-fluoro-phenyl]-amide;
5-(2-Methoxy-ethoxy)-3-methyl-pyrazine-2-carboxylic acid [3-((R)-5-amino-3-difluoromethyl-3,6-dihydro-2H-[1,4]oxazin-3-yl)-4-fluoro-phenyl]-amide;
3,5-Dimethoxy-pyridine-2-carboxylic acid [3-((R)-5-amino-3-difluoromethyl-3,6-dihydro-2H-[1,4]oxazin-3-yl)-4-fluoro-phenyl]-amide;
5-(2-Methoxy-ethoxy)-pyridine-2-carboxylic acid [3-((R)-5-amino-3-difluoromethyl-3,6-dihydro-2H-[1,4]oxazin-3-yl)-4-fluoro-phenyl]-amide;
3-Fluoro-5-(2-methoxy-ethoxy)-pyridine-2-carboxylic acid [3-((R)-5-amino-3-difluoromethyl-3,6-dihydro-2H-[1,4]oxazin-3-yl)-4-fluoro-phenyl]-amide;
5-But-2-ynyloxy-3-methyl-pyrazine-2-carboxylic acid [3-((R)-5-amino-3-difluoromethyl-3,6-dihydro-2H-[1,4]oxazin-3-yl)-4-fluoro-phenyl]-amide;
3-Chloro-5-methoxymethyl-pyridine-2-carboxylic acid [3-((R)-5-amino-3-difluoromethyl-3,6-dihydro-2H-[1,4]oxazin-3-yl)-4-fluoro-phenyl]-amide;
3-Chloro-5-fluoromethoxy-pyridine-2-carboxylic acid [3-((R)-5-amino-3-difluoromethyl-3,6-dihydro-2H-[1,4]oxazin-3-yl)-4-fluoro-phenyl]-amide; and
3-Chloro-5-difluoromethoxy-pyridine-2-carboxylic acid [3-((R)-5-amino-3-difluoromethyl-3,6-dihydro-2H-[1,4]oxazin-3-yl)-4-fluoro-phenyl]-amide,
   or a pharmaceutically acceptable salt thereof.

### Embodiment 17

The method according to any one of Embodiments 1 to 4, wherein the compound is selected from the group consisting of:
5-Chloro-3-methoxymethyl-pyridine-2-carboxylic acid [3-(5-amino-3-difluoromethyl-3,6-dihydro-2H-[1,4]oxazin-3-yl)-4-fluoro-phenyl]-amide;
3-Chloro-5-cyano-pyridine-2-carboxylic acid [3-(5-amino-3-difluoromethyl-3,6-dihydro-2H-[1,4]oxazin-3-yl)-4-fluoro-phenyl]-amide;
3-Amino-5-tris-deutero-methoxy-pyrazine-2-carboxylic acid [3-(5-amino-3-difluoromethyl-3,6-dihydro-2H-[1,4]oxazin-3-yl)-4-fluoro-phenyl]-amide;
3-Amino-5-prop-2-ynyloxy-pyrazine-2-carboxylic acid [3-(5-amino-3-difluoromethyl-3,6-dihydro-2H-[1,4]oxazin-3-yl)-4-fluoro-phenyl]-amide;
3-Chloro-1H-pyrrolo[2,3-b]pyridine-6-carboxylic acid [3-(5-amino-3-difluoromethyl-3,6-dihydro-2H-[1,4]oxazin-3-yl)-4-fluoro-phenyl]-amide;
3-Amino-5-difluoromethyl-pyrazine-2-carboxylic acid [3-(5-amino-3-difluoromethyl-3,6-dihydro-2H-[1,4]oxazin-3-yl)-4-fluoro-phenyl]-amide;
5-Methoxy-3-methyl-pyridine-2-carboxylic acid [3-(5-amino-3-difluoromethyl-3,6-dihydro-2H-[1,4]oxazin-3-yl)-4-fluoro-phenyl]-amide;
5-Difluoromethyl-3-methyl-pyridine-2-carboxylic acid [3-(5-amino-3-difluoromethyl-3,6-dihydro-2H-[1,4]oxazin-3-yl)-4-fluoro-phenyl]-amide;
5-Fluoro-3-trideuteromethoxymethyl-pyridine-2-carboxylic acid [3-(5-amino-3-difluoromethyl-3,6-dihydro-2H-[1,4]oxazin-3-yl)-4-fluoro-phenyl]-amide;
5-Trideuteromethoxy-3-trideuteromethoxymethyl-pyridine-2-carboxylic acid [3-(5-amino-3-difluoromethyl-3,6-dihydro-2H-[1,4]oxazin-3-yl)-4-fluoro-phenyl]-amide;
3-Amino-5-cyano-pyridine-2-carboxylic acid [3-(5-amino-3-difluoromethyl-3,6-dihydro-2H-[1,4]oxazin-3-yl)-4-fluoro-phenyl]-amide;
3-Amino-5-difluoromethyl-pyridine-2-carboxylic acid [3-(5-amino-3-difluoromethyl-3,6-dihydro-2H-[1,4]oxazin-3-yl)-4-fluoro-phenyl]-amide;
5-Trideuteromethoxy-3-methyl-pyridine-2-carboxylic acid [3-(5-amino-3-difluoromethyl-3,6-dihydro-2H-[1,4]oxazin-3-yl)-4-fluoro-phenyl]-amide;
5-Cyano-3-methyl-pyridine-2-carboxylic acid [3-(5-amino-3-difluoromethyl-3,6-dihydro-2H-[1,4]oxazin-3-yl)-4,5-difluoro-phenyl]-amide;
3-Chloro-5-trideuteromethoxy-pyridine-2-carboxylic acid [3-(5-amino-3-difluoromethyl-3,6-dihydro-2H-[1,4]oxazin-3-yl)-4,5-difluoro-phenyl]-amide;
4,6-Dideutero-5-chloro-3-trideuteromethyl-pyridine-2-carboxylic acid [3-(5-amino-3-difluoromethyl-3,6-dihydro-2H-[1,4]oxazin-3-yl)-4,5-difluoro-phenyl]-amide;
3-Chloro-5-cyano-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Chloro-5-methoxy-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Chloro-5-difluoromethoxy-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
5-Chloro-3-methyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
5-Chloro-3-fluoro-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Chloro-5-trideutero-methoxy-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
2,5-Dimethyl-oxazole-4-carboxylic acid [3-(5-amino-3,6-dimethyl-6-trifluoromethyl-3,6-dihydro-2H-[1,4]oxazin-3-yl)-4-fluoro-phenyl]-amide;
5-Difluoromethoxy-3-methyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Amino-5-prop-2-ynyloxy-pyrazine-2-carboxylic acid [3-(5-amino-3,6-dimethyl-6-trifluoromethyl-3,6-dihydro-2H-[1,4]oxazin-3-yl)-4-fluoro-phenyl]-amide;
3-Amino-5-cyano-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
5-Difluoromethyl-3-methyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Amino-5-difluoromethyl-pyrazine-2-carboxylic acid [3-(5-amino-3,6-dimethyl-6-trifluoromethyl-3,6-dihydro-2H-[1,4]oxazin-3-yl)-4-fluoro-phenyl]-amide;
3-Amino-5-difluoromethyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
4-Difluoromethyl-6-methoxy-pyridazine-3-carboxylic acid [3-(5-amino-3,6-dimethyl-6-trifluoromethyl-3,6-dihydro-2H-[1,4]oxazin-3-yl)-4-fluoro-phenyl]-amide;
5-Cyano-3-trideuteromethoxymethyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Chloro-5-difluormethyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Chloro-5-trideuteromethoxy-dideuteromethyl-1H-pyrrolo[2,3-b]pyridine-6-carboxylic acid [3-(5-amino-3,6-dimethyl-6-trifluoromethyl-3,6-dihydro-2H-[1,4]oxazin-3-yl)-4-fluoro-phenyl]-amide;
3-Chloro-5-trifluoromethyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
5-Fluoro-3-methyl-pyridine-2-carboxylic acid [3-(5-amino-3,6-dimethyl-6-trifluoro-methyl-3,6-dihydro-2H-[1,4]oxazin-3-yl)-4-fluoro-phenyl]-amide;
5-Trideuteromethoxy-3-methyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
5-Cyano-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
5-Difluoromethoxy-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
5-Chloro-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
5-Cyano-3-methyl-pyridine-2-carboxylic acid [3-(5-amino-3,6,6-tris-fluoromethyl-3,6-dihydro-2H-[1,4]oxazin-3-yl)-4-fluoro-phenyl]-amide;
5-Difluoromethyl-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
3-Amino-5-difluoromethyl-pyrazine-2-carboxylic acid [3-(5-amino-6,6-bis-fluoromethyl-3-methyl-3,6-dihydro-2H-[1,4]oxazin-3-yl)-4-fluoro-phenyl]-amide;
3-Amino-5-difluoromethyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
3-Amino-5-cyano-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
5-Difluoromethyl-3-methyl-pyridine-2-carboxylic acid [3-(5-amino-3,6,6-tris-fluoromethyl-3,6-dihydro-2H-[1,4]oxazin-3-yl)-4-fluoro-phenyl]-amide;
3-Chloro-5-difluoromethyl-pyridine-2-carboxylic acid [3-(5-amino-3,6,6-tris-fluoromethyl-3,6-dihydro-2H-[1,4]oxazin-3-yl)-4-fluoro-phenyl]-amide;
3-Chloro-5-trifluoromethyl-pyridine-2-carboxylic acid [3-(5-amino-3,6,6-tris-fluoromethyl-3,6-dihydro-2H-[1,4]oxazin-3-yl)-4-fluoro-phenyl]-amide;
3,5-Dichloro-pyridine-2-carboxylic acid [3-(5-amino-3,6,6-tris-fluoromethyl-3,6-dihydro-2H-[1,4]oxazin-3-yl)-4-fluoro-phenyl]-amide;
3-Amino-5-cyano-pyridine-2-carboxylic acid [3-(5-amino-3,6,6-tris-fluoromethyl-3,6-dihydro-2H-[1,4]oxazin-3-yl)-4-fluoro-phenyl]-amide;
3-Chloro-5-cyano-pyridine-2-carboxylic acid [3-(5-amino-3,6,6-tris-fluoromethyl-3,6-dihydro-2H-[1,4]oxazin-3-yl)-4-fluoro-phenyl]-amide;
N-[3-(5-Amino-3,6-dimethyl-6-trifluoromethyl-3,6-dihydro-2H-[1,4]oxazin-3-yl)-4-fluoro-phenyl]-6-methoxy-2-methyl-nicotinamide;
N-[3-(5-Amino-3,6-dimethyl-6-trifluoromethyl-3,6-dihydro-2H-[1,4]oxazin-3-yl)-4-fluoro-phenyl]-6-trideuteromethoxy-2-methyl-nicotinamide;
2-Amino-N-[3-(5-amino-3,6-dimethyl-6-trifluoromethyl-3,6-dihydro-2H-[1,4]oxazin-3-yl)-4-fluoro-phenyl]-6-ethoxy-nicotinamide;
2-Amino-N-[3-(5-amino-3,6-dimethyl-6-trifluoromethyl-3,6-dihydro-2H-[1,4]oxazin-3-yl)-4-fluoro-phenyl]-6-methoxy-nicotinamide;
2-Amino-N-[3-(5-amino-3,6-dimethyl-6-trifluoromethyl-3,6-dihydro-2H-[1,4]oxazin-3-yl)-4-fluoro-phenyl]-6-trideuteromethoxy-nicotinamide;
2-Amino-N-[3-(5-amino-3,6-dimethyl-6-trifluoromethyl-3,6-dihydro-2H-[1,4]oxazin-3-yl)-4-fluoro-phenyl]-6-pentadeuteroethoxy-nicotinamide;
N-[3-(5-Amino-3,6-dimethyl-6-trifluoromethyl-3,6-dihydro-2H-[1,4]oxazin-3-yl)-4-fluoro-phenyl]-2-chloro-6-methoxy-nicotinamide;
N-[3-(5-Amino-3,6-dimethyl-6-trifluoromethyl-3,6-dihydro-2H-[1,4]oxazin-3-yl)-4-fluoro-phenyl]-2-chloro-6-ethoxy-nicotinamide;
2-Amino-N-[3-(5-amino-3,6-dimethyl-6-trifluoromethyl-3,6-dihydro-2H-[1,4]oxazin-3-yl)-4-fluoro-phenyl]-6-cyclopropylmethoxy-nicotinamide; and
2-Amino-N-[3-(5-amino-3,6-dimethyl-6-trifluoromethyl-3,6-dihydro-2H-[1,4]oxazin-3-yl)-4-fluoro-phenyl]-6-(2,2,2-trifluoro-ethoxy)-nicotinamide;
   or a pharmaceutically acceptable salt thereof.

### Embodiment 18

The method according to any one of Embodiments 1 to 4, wherein the compound is selected from the group consisting of:
5-Chloro-3-methoxymethyl-pyridine-2-carboxylic acid [3-((R)-5-amino-3-difluoromethyl-3,6-dihydro-2H-[1,4]oxazin-3-yl)-4-fluoro-phenyl]-amide;
3-Chloro-5-cyano-pyridine-2-carboxylic acid [3-((R)-5-amino-3-difluoromethyl-3,6-dihydro-2H-[1,4]oxazin-3-yl)-4-fluoro-phenyl]-amide;
3-Amino-5-tris-deutero-methoxy-pyrazine-2-carboxylic acid [3-((R)-5-amino-3-difluoromethyl-3,6-dihydro-2H-[1,4]oxazin-3-yl)-4-fluoro-phenyl]-amide;
3-Amino-5-prop-2-ynyloxy-pyrazine-2-carboxylic acid [3-((R)-5-amino-3-difluoromethyl-3,6-dihydro-2H-[1,4]oxazin-3-yl)-4-fluoro-phenyl]-amide;
3-Chloro-1H-pyrrolo[2,3-b]pyridine-6-carboxylic acid [3-((R)-5-amino-3-difluoromethyl-3,6-dihydro-2H-[1,4]oxazin-3-yl)-4-fluoro-phenyl]-amide;
3-Amino-5-difluoromethyl-pyrazine-2-carboxylic acid [3-((R)-5-amino-3-difluoromethyl-3,6-dihydro-2H-[1,4]oxazin-3-yl)-4-fluoro-phenyl]-amide;
5-Methoxy-3-methyl-pyridine-2-carboxylic acid [3-((R)-5-amino-3-difluoromethyl-3,6-dihydro-2H-[1,4]oxazin-3-yl)-4-fluoro-phenyl]-amide;
5-Difluoromethyl-3-methyl-pyridine-2-carboxylic acid [3-((R)-5-amino-3-difluoromethyl-3,6-dihydro-2H-[1,4]oxazin-3-yl)-4-fluoro-phenyl]-amide;
5-Fluoro-3-trideuteromethoxymethyl-pyridine-2-carboxylic acid [3-((R)-5-amino-3-difluoromethyl-3,6-dihydro-2H-[1,4]oxazin-3-yl)-4-fluoro-phenyl]-amide;
5-Trideuteromethoxy-3-trideuteromethoxymethyl-pyridine-2-carboxylic acid [3-((R)-5-amino-3-difluoromethyl-3,6-dihydro-2H-[1,4]oxazin-3-yl)-4-fluoro-phenyl]-amide;
3-Amino-5-cyano-pyridine-2-carboxylic acid [3-((R)-5-amino-3-difluoromethyl-3,6-dihydro-2H-[1,4]oxazin-3-yl)-4-fluoro-phenyl]-amide;
3-Amino-5-difluoromethyl-pyridine-2-carboxylic acid [3-((R)-5-amino-3-difluoromethyl-3,6-dihydro-2H-[1,4]oxazin-3-yl)-4-fluoro-phenyl]-amide;
5-Trideuteromethoxy-3-methyl-pyridine-2-carboxylic acid [3-((R)-5-amino-3-difluoromethyl-3,6-dihydro-2H-[1,4]oxazin-3-yl)-4-fluoro-phenyl]-amide;
5-Cyano-3-methyl-pyridine-2-carboxylic acid [3-((R)-5-amino-3-difluoromethyl-3,6-dihydro-2H-[1,4]oxazin-3-yl)-4,5-difluoro-phenyl]-amide;
3-Chloro-5-trideuteromethoxy-pyridine-2-carboxylic acid [3-((R)-5-amino-3-difluoromethyl-3,6-dihydro-2H-[1,4]oxazin-3-yl)-4,5-difluoro-phenyl]-amide;
4,6-Dideutero-5-chloro-3-trideuteromethyl-pyridine-2-carboxylic acid [3-((R)-5-amino-3-difluoromethyl-3,6-dihydro-2H-[1,4]oxazin-3-yl)-4,5-difluoro-phenyl]-amide;
3-Chloro-5-cyano-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Chloro-5-methoxy-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Chloro-5-difluoromethoxy-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Chloro-3-methyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Chloro-3-fluoro-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Chloro-5-trideutero-methoxy-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
2,5-Dimethyl-oxazole-4-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Difluoromethoxy-3-methyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Amino-5-prop-2-ynyloxy-pyrazine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Amino-5-cyano-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Difluoromethyl-3-methyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Amino-5-difluoromethyl-pyrazine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Amino-5-difluoromethyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
4-Difluoromethyl-6-methoxy-pyridazine-3-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Cyano-3-trideuteromethoxymethyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Chloro-5-difluormethyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Chloro-5-trideuteromethoxy-dideuteromethyl-1H-pyrrolo[2,3-b]pyridine-6-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Chloro-5-trifluoromethyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Fluoro-3-methyl-pyridine-2-carboxylic acid [3-((3R,6R)-5-amino-3,6-dimethyl-6-trifluoro-methyl-3,6-dihydro-2-[1,4]oxazin-3-yl)-4-fluoro-phenyl]-amide;
5-Trideuteromethoxy-3-methyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Cyano-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
5-Cyano-3-methyl-pyridine-2-carboxylic acid [3-((R)-5-amino-6,6-bis-fluoromethyl-3-methyl-3,6-dihydro-2H-[1,4]oxazin-3-yl)-4-fluoro-phenyl]-amide;
5-Difluoromethoxy-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
5-Difluoromethoxy-3-methyl-pyridine-2-carboxylic acid [3-((R)-5-amino-6,6-bis-fluoromethyl-3-methyl-3,6-dihydro-2H-[1,4]oxazin-3-yl)-4-fluoro-phenyl]-amide;
5-Chloro-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
5-Cyano-3-methyl-pyridine-2-carboxylic acid [3-((R)-5-amino-3,6,6-tris-fluoromethyl-3,6-dihydro-2H-[1,4]oxazin-3-yl)-4-fluoro-phenyl]-amide;
5-Difluoromethyl-3-methyl-pyridine-2-carboxylic acid [3-((R)-5-amino-6,6-bis-fluoromethyl-3-methyl-3,6-dihydro-2H-[1,4]oxazin-3-yl)-4-fluoro-phenyl]-amide;
3-Amino-5-difluoromethyl-pyrazine-2-carboxylic acid [3-((R)-5-amino-6,6-bis-fluoromethyl-3-methyl-3,6-dihydro-2H-[1,4]oxazin-3-yl)-4-fluoro-phenyl]-amide;
3-Amino-5-difluoromethyl-pyridine-2-carboxylic acid [3-((R)-5-amino-6,6-bis-fluoromethyl-3-methyl-3,6-dihydro-2H-[1,4]oxazin-3-yl)-4-fluoro-phenyl]-amide;
3-Amino-5-cyano-pyridine-2-carboxylic acid [3-((R)-5-amino-6,6-bis-fluoromethyl-3-methyl-3,6-dihydro-2H-[1,4]oxazin-3-yl)-4-fluoro-phenyl]-amide;
5-Difluoromethyl-3-methyl-pyridine-2-carboxylic acid [3-((R)-5-amino-3,6,6-tris-fluoromethyl-3,6-dihydro-2H-[1,4]oxazin-3-yl)-4-fluoro-phenyl]-amide;
3-Chloro-5-difluoromethyl-pyridine-2-carboxylic acid [3-((R)-5-amino-3,6,6-tris-fluoromethyl-3,6-dihydro-2H-[1,4]oxazin-3-yl)-4-fluoro-phenyl]-amide;
3-Chloro-5-trifluoromethyl-pyridine-2-carboxylic acid [3-((R)-5-amino-3,6,6-tris-fluoromethyl-3,6-dihydro-2H-[1,4]oxazin-3-yl)-4-fluoro-phenyl]-amide;
3,5-Dichloro-pyridine-2-carboxylic acid [3-((R)-5-amino-3,6,6-tris-fluoromethyl-3,6-dihydro-2H-[1,4]oxazin-3-yl)-4-fluoro-phenyl]-amide;
3-Amino-5-cyano-pyridine-2-carboxylic acid [3-((R)-5-amino-3,6,6-tris-fluoromethyl-3,6-dihydro-2H-[1,4]oxazin-3-yl)-4-fluoro-phenyl]-amide;
3-Chloro-5-cyano-pyridine-2-carboxylic acid [3-((R)-5-amino-3,6,6-tris-fluoromethyl-3,6-dihydro-2H-[1,4]oxazin-3-yl)-4-fluoro-phenyl]-amide;
N-[3-((3R,6R)-5-Amino-3,6-dimethyl-6-trifluoromethyl-3,6-dihydro-2H-[1,4]oxazin-3-yl)-4-fluoro-phenyl]-6-methoxy-2-methyl-nicotinamide;
N-[3-((3R,6R)-5-Amino-3,6-dimethyl-6-trifluoromethyl-3,6-dihydro-2H-[1,4]oxazin-3-yl)-4-fluoro-phenyl]-6-trideuteromethoxy-2-methyl-nicotinamide;
2-Amino-N-[3-((3R,6R)-5-amino-3,6-dimethyl-6-trifluoromethyl-3,6-dihydro-2H-[1,4]oxazin-3-yl)-4-fluoro-phenyl]-6-ethoxy-nicotinamide;
2-Amino-N-[3-((3R,6R)-5-amino-3,6-dimethyl-6-trifluoromethyl-3,6-dihydro-2H-[1,4]oxazin-3-yl)-4-fluoro-phenyl]-6-methoxy-nicotinamide;
2-Amino-N-[3-((3R,6R)-5-amino-3,6-dimethyl-6-trifluoromethyl-3,6-dihydro-2H-[1,4]oxazin-3-yl)-4-fluoro-phenyl]-6-trideuteromethoxy-nicotinamide;
2-Amino-N-[3-((3R,6R)-5-amino-3,6-dimethyl-6-trifluoromethyl-3,6-dihydro-2H-[1,4]oxazin-3-yl)-4-fluoro-phenyl]-6-pentadeuteroethoxy-nicotinamide;
N-[3-((3R,6R)-5-Amino-3,6-dimethyl-6-trifluoromethyl-3,6-dihydro-2H-[1,4]oxazin-3-yl)-4-fluoro-phenyl]-2-chloro-6-methoxy-nicotinamide;
N-[3-((3R,6R)-5-Amino-3,6-dimethyl-6-trifluoromethyl-3,6-dihydro-2H-[1,4]oxazin-3-yl)-4-fluoro-phenyl]-2-chloro-6-ethoxy-nicotinamide;
2-Amino-N-[3-((3R,6R)-5-amino-3,6-dimethyl-6-trifluoromethyl-3,6-dihydro-2H-[1,4]oxazin-3-yl)-4-fluoro-phenyl]-6-cyclopropylmethoxy-nicotinamide; and
2-Amino-N-[3-((3R,6R)-5-amino-3,6-dimethyl-6-trifluoromethyl-3,6-dihydro-2H-[1,4]oxazin-3-yl)-4-fluoro-phenyl]-6-(2,2,2-trifluoro-ethoxy)-nicotinamide;
   or a pharmaceutically acceptable salt thereof.

### Embodiment 19

A compound according to formula (I):

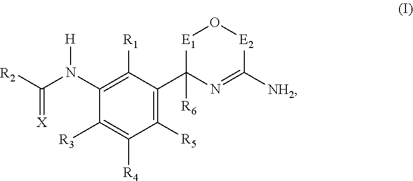

or a pharmaceutically acceptable salt thereof, wherein:
X is O or S;
R₁ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
R₂ is an aryl, heteroaryl or non-aromatic heterocyclyl group G₁, which group G₁ is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3 or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
either
R₄ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl; and
R₅ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ is hydrogen; (C₁₋₈)alkyl; halogen-(C₁₋₈)alkyl; hydroxy-(C₁₋₈)alkyl; (C₁₋₈)alkoxy-(C₁₋₈)alkyl; mercapto-(C₁₋₈)alkyl; (C₁₋₈)alkylthio-(C₁₋₈)alkyl; amino-(C₁₋₈)alkyl; N—(C₁₋₈)alkylamino-(C₁₋₈)alkyl; N,N-di-[(C₁₋₈)alkyl]amino-(C₁₋₈)alkyl with two identical or different (C₁₋₈)alkyl moieties in the N,N-di-[(C₁₋₈)alkyl]amino moiety; (C₂₋₈)alkenyl; or (C₂₋₈)alkynyl;
E₁ is —C(R₇)(R₈)—; or —C(R₇)(R₈)—C(R₉)(R₁₀)—;
E₂ is —C(R₁₁)(R₁₂)—; or —C(R₁₁)(R₁₂)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ and R₁₂ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ and R₁₂, taken together, are oxo or —CH₂—CH₂—; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
   R₁₃ and R₁₄, taken together, are oxo or —CH₂—CH₂—,
for use in the treatment of a disorder or disease selected from the group consisting of insulin resistance, impaired glucose tolerance, type 2 diabetes, obesity, hypertension, and diabetic complications.

### Embodiment 20

Use of a compound according to formula (I):

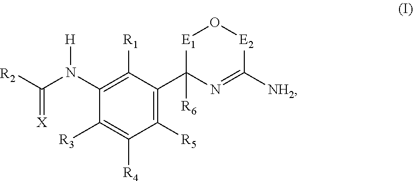

or a pharmaceutically acceptable salt thereof, wherein:
X is O or S;
R₁ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
R₂ is an aryl, heteroaryl or non-aromatic heterocyclyl group G₁, which group G₁ is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3 or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
either
R₄ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl; and
R₅ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ is hydrogen; (C₁₋₈)alkyl; halogen-(C₁₋₈)alkyl; hydroxy-(C₁₋₈)alkyl; (C₁₋₈)alkoxy-(C₁₋₈)alkyl; mercapto-(C₁₋₈)alkyl; (C₁₋₈)alkylthio-(C₁₋₈)alkyl; amino-(C₁₋₈)alkyl; N—(C₁₋₈)alkylamino-(C₁₋₈)alkyl; N,N-di-[(C₁₋₈)alkyl]amino-(C₁₋₈)alkyl with two identical or different (C₁₋₈)alkyl moieties in the N,N-di-[(C₁₋₈)alkyl]amino moiety; (C₂₋₈)alkenyl; or (C₂₋₈)alkynyl;
E₁ is —C(R₇)(R₈)—; or —C(R₇)(R₈)—C(R₉)(R₁₀)—;
E₂ is —C(R₁₁)(R₁₂)—; or —C(R₁₁)(R₁₂)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—; either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ and R₁₂ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ and R₁₂, taken together, are oxo or —CH₂—CH₂—; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
   R₁₃ and R₁₄, taken together, are oxo or —CH₂—CH₂—,
in the manufacture of a medicament for the treatment of a disorder or disease selected from the group consisting of insulin resistance, impaired glucose tolerance, type 2 diabetes, obesity, hypertension, and diabetic complications.

### Embodiment 21

A combination comprising a therapeutically effective amount of a compound according to formula (I):

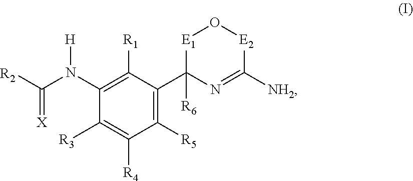

or a pharmaceutically acceptable salt thereof, wherein:
X is O or S;
R₁ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
R₂ is an aryl, heteroaryl or non-aromatic heterocyclyl group G₁, which group G₁ is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3 or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
either
R₄ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl; and
R₅ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ is hydrogen; (C₁₋₈)alkyl; halogen-(C₁₋₈)alkyl; hydroxy-(C₁₋₈)alkyl; (C₁₋₈)alkoxy-(C₁₋₈)alkyl; mercapto-(C₁₋₈)alkyl; (C₁₋₈)alkylthio-(C₁₋₈)alkyl; amino-(C₁₋₈)alkyl; N—(C₁₋₈)alkylamino-(C₁₋₈)alkyl; N,N-di-[(C₁₋₈)alkyl]amino-(C₁₋₈)alkyl with two identical or different (C₁₋₈)alkyl moieties in the N,N-di-[(C₁₋₈)alkyl]amino moiety; (C₂₋₈)alkenyl; or (C₂₋₈)alkynyl;
E₁ is —C(R₇)(R₈)—; or —C(R₇)(R₈)—C(R₉)(R₁₀)—;
E₂ is —C(R₁₁)(R₁₂)—; or —C(R₁₁)(R₁₂)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ and R₁₂ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ and R₁₂, taken together, are oxo or —CH₂—CH₂—; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
   R₁₃ and R₁₄, taken together, are oxo or —CH₂—CH₂—, and one or more therapeutically active co-agents selected from the group consisting of:
a) antidiabetic agents,
b) hypolipidemic agents,
c) anti-obesity agents,
d) anti-hypertensive agents, and
e) agonists of peroxisome proliferator-activator receptors.

### Embodiment 22

A pharmaceutical compositions comprising:
i) a compound according to formula (I):

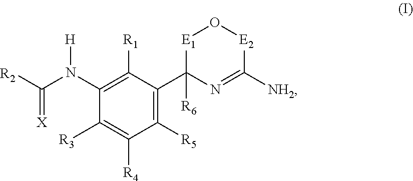

or a pharmaceutically acceptable salt thereof, wherein:
X is O or S;
R₁ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
R₂ is an aryl, heteroaryl or non-aromatic heterocyclyl group G₁, which group G₁ is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3 or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
either
R₄ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl; and
R₅ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ is hydrogen; (C₁₋₈)alkyl; halogen-(C₁₋₈)alkyl; hydroxy-(C₁₋₈)alkyl; (C₁₋₈)alkoxy-(C₁₋₈)alkyl; mercapto-(C₁₋₈)alkyl; (C₁₋₈)alkylthio-(C₁₋₈)alkyl; amino-(C₁₋₈)alkyl; N—(C₁₋₈)alkylamino-(C₁₋₈)alkyl; N,N-di-[(C₁₋₈)alkyl]amino-(C₁₋₈)alkyl with two identical or different (C₁₋₈)alkyl moieties in the N,N-di-[(C₁₋₈)alkyl]amino moiety; (C₂₋₈)alkenyl; or (C₂₋₈)alkynyl;
E₁ is —C(R₇)(R₈)—; or —C(R₇)(R₈)—C(R₉)(R₁₀)—;
E₂ is —C(R₁₁)(R₁₂)—; or —C(R₁ᵢ)(R₁₂)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ and R₁₂ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ and R₁₂, taken together, are oxo or —CH₂—CH₂—; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—, and
ii) at least one compound selected from
   a) antidiabetic agents,
   b) hypolipidemic agents,
   c) anti-obesity agents,
   d) anti-hypertensive agents,
   e) agonists of peroxisome proliferator-activator receptors, and
iii) one or more pharmaceutically acceptable carriers.

### Embodiment 23

A method of modulating BACE-2 activity in a subject, wherein the method comprises administering to the subject a therapeutically effective amount of a compound of formula (II):

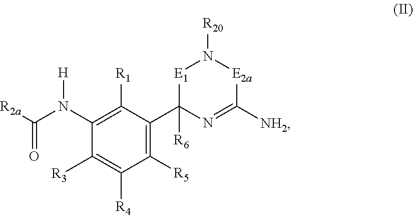

or a pharmaceutically acceptable salt thereof, wherein:
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₂ₐ is an aryl, heteroaryl or non-aromatic heterocyclyl group G₁, which group G₁ is optionally substituted by 1 to 4 substituents independently selected from the group, consisting of cyano, amino, aminocarbonyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
either
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl; and
R₅ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₈)alkylamino-(C₁₋₈)alkyl, N,N-di-[(C₁₋₈)alkyl]amino-(C₁₋₈)alkyl with two identical or different (C₁₋₈)alkyl moieties in the N,N-di-[(C₁₋₈)alkyl]amino moiety, (C₂₋₈)alkenyl, or (C₂₋₈)alkynyl;
R₂₀ is hydrogen, (C₁₋₈)alkyl, (C₁₋₈)alkyl substituted by halogen, (C₃₋₈)cycloalkyl-(C₁₋₈)alkyl, (C₃₋₈)cycloalkoxy-(C₁₋₈)alkyl, aryloxy-(C₁₋₈)alkyl, (C₁₋₈)alkoxy-(C₁₋₈)alkyl, (C₁₋₈)alkylthio-(C₁₋₈)alkyl, (C₁₋₈)alkylsulfinyl, (C₁₋₈)alkylsulfinyl-(C₁₋₈)alkyl, (C₁₋₈)alkylsulfonyl, (C₁₋₈)alkylsulfonyl-(C₁₋₈)alkyl, amino-(C₁₋₈)alkyl, (C₁₋₈)alkylamino-(C₁₋₈)alkyl, di(C₁₋₈)alkylamino-(C₁₋₈)alkyl with two identical or different (C₁₋₈)alkyl moieties in the di(C₁₋₈)alkylamino moiety, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyl-(C₁₋₈)alkyl, (C₁₋₈)alkylcarbonyl-(C₁₋₈)alkyl, (C₁₋₈)alkoxycarbonyl, halogen-(C₁₋₈)alkoxycarbonyl, (C₁₋₈)alkoxycarbonyl-(C₁₋₈)alkyl, (C₁₋₈)alkoxy-(C₁₋₈)alkylcarbonyl, or a (C₃₋₈)cycloalkylcarbonyl, arylcarbonyl, aryl-(C₁₋₈)alkylcarbonyl, heteroarylcarbonyl, heteroaryl-(C₁₋₈)alkylcarbonyl, non-aromatic heterocyclylcarbonyl, (C₃₋₈)cycloalkylsulfonyl, arylsulfonyl, aryl-(C₁₋₈)alkylsulfonyl, heteroarylsulfonyl, heteroaryl-(C₁₋₈)alkylsulfonyl, non-aromatic heterocyclylsulfonyl, (C₃₋₈)cycloalkyl, aryl, aryl-(C₁₋₈)alkyl, heteroaryl, heteroaryl-(C₁₋₈)alkyl or non-aromatic heterocyclyl group G₃, which group G₃ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₄, which group G₄ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or

R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₆R₁₇—CR₁₈R₁₉—
wherein R₁₆, R₁₇, R₁₈ and R₁₉ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
   R₁₃ and R₁₄, taken together, are oxo or —CH₂—CH₂—.

### Embodiment 24

A method of treating a disorder or disease associated with inhibition of BACE-2 activity, comprising administering to the subject a therapeutically effective amount of a compound of formula (II):

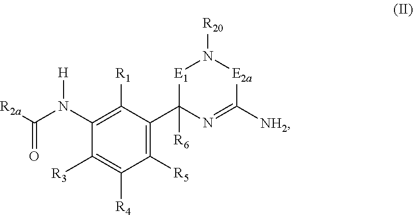

or a pharmaceutically acceptable salt thereof, wherein:

R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;

R₂ₐ is an aryl, heteroaryl or non-aromatic heterocyclyl group G₁, which group G₁ is optionally substituted by 1 to 4 substituents independently selected from the group, consisting of cyano, amino, aminocarbonyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
either
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl; and
R₅ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₈)alkylamino-(C₁₋₈)alkyl, N,N-di-[(C₁₋₈)alkyl]amino-(C₁₋₈)alkyl with two identical or different (C₁₋₈)alkyl moieties in the N,N-di-[(C₁₋₈)alkyl]amino moiety, (C₂₋₈)alkenyl, or (C₂₋₈)alkynyl;
R₂₀ is hydrogen, (C₁₋₈)alkyl, (C₁₋₈)alkyl substituted by halogen, (C₃₋₈)cycloalkyl-(C₁₋₈)alkyl, (C₃₋₈)cycloalkoxy-(C₁₋₈)alkyl, aryloxy-(C₁₋₈)alkyl, (C₁₋₈)alkoxy-(C₁₋₈)alkyl, (C₁₋₈)alkylthio-(C₁₋₈)alkyl, (C₁₋₈)alkylsulfinyl, (C₁₋₈)alkylsulfinyl-(C₁₋₈)alkyl, (C₁₋₈)alkylsulfonyl, (C₁₋₈)alkylsulfonyl-(C₁₋₈)alkyl, amino-(C₁₋₈)alkyl, (C₁₋₈)alkylamino-(C₁₋₈)alkyl, di(C₁₋₈)alkylamino-(C₁₋₈)alkyl with two identical or different (C₁₋₈)alkyl moieties in the di(C₁₋₈)alkylamino moiety, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyl-(C₁₋₈)alkyl, (C₁₋₈)alkylcarbonyl-(C₁₋₈)alkyl, (C₁₋₈)alkoxycarbonyl, halogen-(C₁₋₈)alkoxycarbonyl, (C₁₋₈)alkoxycarbonyl-(C₁₋₈)alkyl, (C₁₋₈)alkoxy-(C₁₋₈)alkylcarbonyl, or a (C₃₋₈)cycloalkylcarbonyl, arylcarbonyl, aryl-(C₁₋₈)alkylcarbonyl, heteroarylcarbonyl, heteroaryl-(C₁₋₈)alkylcarbonyl, non-aromatic heterocyclylcarbonyl, (C₃₋₈)cycloalkylsulfonyl, arylsulfonyl, aryl-(C₁₋₈)alkylsulfonyl, heteroarylsulfonyl, heteroaryl-(C₁₋₈)alkylsulfonyl, non-aromatic heterocyclylsulfonyl, (C₃₋₈)cycloalkyl, aryl, aryl-(C₁₋₈)alkyl, heteroaryl, heteroaryl-(C₁₋₈)alkyl or non-aromatic heterocyclyl group G₃, which group G₃ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₄, which group G₄ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and Rᵤₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₆R₁₇—CR₁₈R₁₉—
wherein R₁₆, R₁₇, R₁₈ and R₁₉ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
   R₁₃ and R₁₄, taken together, are oxo or —CH₂—CH₂—.

### Embodiment 25

The method according to Embodiment 24 wherein the disorder or disease selected from the group consisting of insulin resistance, impaired glucose tolerance, type 2 diabetes, obesity, hypertension, and diabetic complications.

### Embodiment 26

The method according to Embodiment 25 wherein the disorder or disease selected from the group consisting of impaired glucose tolerance and Type 2 diabetes.

### Embodiment 27

The method according to any one of Embodiments 23 to 26, wherein R₁ is hydrogen.

### Embodiment 28

The method according to any one of Embodiments 23 to 27, wherein R₂ₐ is phenyl or a 5- or 6-membered heteroaryl group G₁ in which structure 1, 2, 3, or 4 ring members are hetero ring members independently selected from the group consisting of a nitrogen ring member, an oxygen ring member and a sulfur ring member, which group G₁ is optionally substituted by 1, 2, 3 or 4 substituents independently selected from the group, consisting of cyano, amino, aminocarbonyl, halogen, (C₁₋₄)alkyl, halogen-(C₁₋₄)alkyl, hydroxy, oxo, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, (C₂₋₄)alkynyl, (C₂₋₄)alkenoxy, and (C₂₋₄)alkynoxy.

### Embodiment 29

The method according to any one of Embodiments 23 to 28, wherein R₃ is hydrogen.

### Embodiment 30

The method according to any one of Embodiments 23 to 29, wherein R₄ is hydrogen, or halogen; and R₅ is hydrogen, or halogen.

### Embodiment 31

The method according to any one of Embodiments 23 to 30, wherein R₆ is (C₁₋₃)alkyl, or halogen-(C₁₋₃)alkyl.

### Embodiment 32

The method according to any one of Embodiments 23 to 31, wherein R₂₀ is hydrogen, (C₁₋₆)alkyl, halogen-(C₁₋₆)alkyl, (C₁₋₄)alkoxy-(C₁₋₄)alkyl, (C₁₋₆)alkylcarbonyl, (C₁₋₆)alkoxycarbonyl, halogen-(C₁₋₆)alkoxycarbonyl, (C₁₋₆)alkoxy-(C₁₋₆)alkylcarbonyl, (C₃₋₆)cycloalkyl, (C₃₋₆)cycloalkyl-carbonyl, or a heteroaryl group optionally substituted by 1, 2, or 3 substituents independently selected from the group consisting of cyano, halogen, hydroxyl, (C₁₋₄)alkyl, halogen-(C₁₋₄)alkyl, (C₁₋₄)alkoxy, halogen-(C₁₋₄)alkoxy, (C₁₋₃)alkoxy-(C₁₋₃)alkyl and (C₁₋₃)alkoxy-(C₁₋₃)alkoxy.

### Embodiment 33

The method according to any one of Embodiments 23 to 32, wherein E₁ is —C(R₇)(R₈)— and

either
each of R₇ and R₈ is independently selected from the group, consisting of hydrogen, cyano, halogen, (C₁₋₃)alkyl and halogen-(C₁₋₃)alkyl;
or
R₇ and R₈, taken together, are oxo or —CH₂—CH₂—.

### Embodiment 34

The method according to any one of Embodiments 23 to 33, wherein E₂ₐ is —C(R₁₁ₐ)(R₁₂ₐ)— and

either
each of R₁₁ₐ and R₁₂ₐ is independently selected from the group, consisting of hydrogen, cyano, halogen, (C₁₋₃)alkyl and halogen-(C₁₋₃)alkyl;
or
R₁₁ₐ and R₁₂ₐ, taken together, are oxo or —CH₂—CH₂—.

### Embodiment 35

The method according to Embodiment 23, wherein the compound is selected from the group consisting of:
5-Bromo-pyridine-2-carboxylic acid [3-(6-amino-2,4-dimethyl-3-oxo-2,3,4,5-tetrahydro-pyrazin-2-yl)-4-fluoro-phenyl]-amide;
5-Bromo-pyridine-2-carboxylic acid [3-(6-amino-2,4-dimethyl-2,3,4,5-tetrahydro-pyrazin-2-yl)-4-fluoro-phenyl]-amide;
5-Bromo-pyridine-2-carboxylic acid [3-(6-amino-2,4-dimethyl-3-oxo-2,3,4,5-tetrahydro-pyrazin-2-yl)-phenyl]-amide;
5-Bromo-pyridine-2-carboxylic acid [3-(6-amino-2,4-dimethyl-5-oxo-2,3,4,5-tetrahydro-pyrazin-2-yl)-4-fluoro-phenyl]-amide;
5-Chloro-pyridine-2-carboxylic acid [3-(6-amino-2,4-dimethyl-5-oxo-2,3,4,5-tetrahydro-pyrazin-2-yl)-4-fluoro-phenyl]-amide;
5-Bromo-pyridine-2-carboxylic acid [3-(6-amino-4-ethyl-2-methyl-5-oxo-2,3,4,5-tetrahydro-pyrazin-2-yl)-4-fluoro-phenyl]-amide;
3,5-Dichloro-pyridine-2-carboxylic acid [3-(6-amino-2,4-dimethyl-5-oxo-2,3,4,5-tetrahydro-pyrazin-2-yl)-4-fluoro-phenyl]-amide;
3,5-Dichloro-pyridine-2-carboxylic acid [3-(6-amino-4-ethyl-2-methyl-5-oxo-2,3,4,5-tetrahydro-pyrazin-2-yl)-4-fluoro-phenyl]-amide;
5-Bromo-pyridine-2-carboxylic acid [3-(6-amino-4-isopropyl-2-methyl-5-oxo-2,3,4,5-tetrahydro-pyrazin-2-yl)-4-fluoro-phenyl]-amide;
3,5-Dichloro-pyridine-2-carboxylic acid {3-[6-amino-4-(2-methoxy-ethyl)-2-methyl-5-oxo-2,3,4,5-tetrahydro-pyrazin-2-yl]-4-fluoro-phenyl}-amide;
5-Bromo-pyridine-2-carboxylic acid {3-[6-amino-4-(2-methoxy-ethyl)-2-methyl-5-oxo-2,3,4,5-tetrahydro-pyrazin-2-yl]-4-fluoro-phenyl}-amide;
5-Bromo-pyridine-2-carboxylic acid {3-[6-amino-2-methyl-4-(1-methyl-1H-pyrazol-4-yl)-5-oxo-2,3,4,5-tetrahydro-pyrazin-2-yl]-4-fluoro-phenyl}-amide;
5-Bromo-pyridine-2-carboxylic acid [3-(6-amino-2-methyl-5-oxo-4-pyridin-3-yl-2,3,4,5-tetrahydro-pyrazin-2-yl)-4-fluoro-phenyl]-amide;
5-Cyano-pyridine-2-carboxylic acid [3-(6-amino-4-ethyl-2-methyl-5-oxo-2,3,4,5-tetrahydro-pyrazin-2-yl)-4-fluoro-phenyl]-amide;
5-Bromo-pyridine-2-carboxylic acid [3-(6-amino-5-ethyl-2,4-dimethyl-3-oxo-2,3,4,5-tetrahydro-pyrazin-2-yl)-phenyl]-amide;
5-Bromo-pyridine-2-carboxylic acid [3-(6-amino-2-difluoromethyl-2,3,4,5-tetrahydro-pyrazin-2-yl)-4-fluoro-phenyl]-amide;
5-Cyano-pyridine-2-carboxylic acid [3-(6-amino-2-difluoromethyl-2,3,4,5-tetrahydro-pyrazin-2-yl)-4-fluoro-phenyl]-amide;
5-Cyano-pyridine-2-carboxylic acid [3-(6-amino-2-difluoromethyl-5-oxo-2,3,4,5-tetrahydro-pyrazin-2-yl)-4-fluoro-phenyl]-amide;
5-Amino-3-{5-[(5-bromo-pyridine-2-carbonyl)-amino]-2-fluoro-phenyl}-3-difluoromethyl-3,6-dihydro-2H-pyrazine-1-carboxylic acid methyl ester;
5-Amino-3-{5-[(5-cyano-pyridine-2-carbonyl)-amino]-2-fluoro-phenyl}-3-difluoromethyl-3,6-dihydro-2H-pyrazine-1-carboxylic acid methyl ester;
5-Bromo-3-methyl-pyridine-2-carboxylic acid [3-(4-acetyl-6-amino-2-difluoromethyl-2,3,4,5-tetrahydro-pyrazin-2-yl)-4-fluoro-phenyl]-amide;
5-Amino-3-{5-[(5-bromo-3-methyl-pyridine-2-carbonyl)-amino]-2-fluoro-phenyl}-3-difluoromethyl-3,6-dihydro-2H-pyrazine-1-carboxylic acid 2,2-dichloro-ethyl ester;
5-Bromo-3-methyl-pyridine-2-carboxylic acid {3-[6-amino-2-difluoromethyl-4-(2-methoxy-acetyl)-2,3,4,5-tetrahydro-pyrazin-2-yl]-4-fluoro-phenyl}-amide;
5-Bromo-3-methyl-pyridine-2-carboxylic acid [3-(6-amino-4-cyclopropanecarbonyl-2-difluoromethyl-2,3,4,5-tetrahydro-pyrazin-2-yl)-4-fluoro-phenyl]-amide;
5-Bromo-3-methyl-pyridine-2-carboxylic acid [3-(6-amino-2-difluoromethyl-2,3,4,5-tetrahydro-pyrazin-2-yl)-4-fluoro-phenyl]-amide;
5-Difluoromethoxy-3-methyl-pyridine-2-carboxylic acid [3-(4-acetyl-6-amino-2-difluoromethyl-2,3,4,5-tetrahydro-pyrazin-2-yl)-4-fluoro-phenyl]-amide;
5-Difluoromethoxy-3-methyl-pyridine-2-carboxylic acid [3-(6-amino-2-difluoromethyl-2,3,4,5-tetrahydro-pyrazin-2-yl)-4-fluoro-phenyl]-amide;
3-Amino-5-methoxy-pyrazine-2-carboxylic acid [3-(6-amino-2-difluoromethyl-2,3,4,5-tetrahydro-pyrazin-2-yl)-4-fluoro-phenyl]-amide; and
3-Amino-5-oxo-4,5-dihydro-pyrazine-2-carboxylic acid [3-(6-amino-2-difluoromethyl-2,3,4,5-tetrahydro-pyrazin-2-yl)-4-fluoro-phenyl]-amide,
   or a pharmaceutically acceptable salt thereof.

### Embodiment 36

A compound according to formula (II):

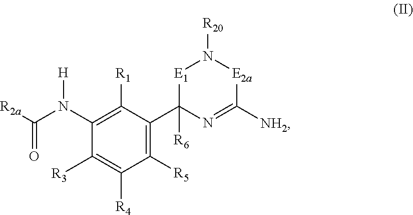

or a pharmaceutically acceptable salt thereof, wherein:
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₂ₐ is an aryl, heteroaryl or non-aromatic heterocyclyl group G₁, which group G₁ is optionally substituted by 1 to 4 substituents independently selected from the group, consisting of cyano, amino, aminocarbonyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
either
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl; and
R₅ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₈)alkylamino-(C₁₋₈)alkyl, N,N-di-[(C₁₋₈)alkyl]amino-(C₁₋₈)alkyl with two identical or different (C₁₋₈)alkyl moieties in the N,N-di-[(C₁₋₈)alkyl]amino moiety, (C₂₋₈)alkenyl, or (C₂₋₈)alkynyl;
R₂₀ is hydrogen, (C₁₋₈)alkyl, (C₁₋₈)alkyl substituted by halogen, (C₃₋₈)cycloalkyl-(C₁₋₈)alkyl, (C₃₋₈)cycloalkoxy-(C₁₋₈)alkyl, aryloxy-(C₁₋₈)alkyl, (C₁₋₈)alkoxy-(C₁₋₈)alkyl, (C₁₋₈)alkylthio-(C₁₋₈)alkyl, (C₁₋₈)alkylsulfinyl, (C₁₋₈)alkylsulfinyl-(C₁₋₈)alkyl, (C₁₋₈)alkylsulfonyl, (C₁₋₈)alkylsulfonyl-(C₁₋₈)alkyl, amino-(C₁₋₈)alkyl, (C₁₋₈)alkylamino-(C₁₋₈)alkyl, di(C₁₋₈)alkylamino-(C₁₋₈)alkyl with two identical or different (C₁₋₈)alkyl moieties in the di(C₁₋₈)alkylamino moiety, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyl-(C₁₋₈)alkyl, (C₁₋₈)alkylcarbonyl-(C₁₋₈)alkyl, (C₁₋₈)alkoxycarbonyl, halogen-(C₁₋₈)alkoxycarbonyl, (C₁₋₈)alkoxycarbonyl-(C₁₋₈)alkyl, (C₁₋₈)alkoxy-(C₁₋₈)alkylcarbonyl, or a (C₃₋₈)cycloalkylcarbonyl, arylcarbonyl, aryl-(C₁₋₈)alkylcarbonyl, heteroarylcarbonyl, heteroaryl-(C₁₋₈)alkylcarbonyl, non-aromatic heterocyclylcarbonyl, (C₃₋₈)cycloalkylsulfonyl, arylsulfonyl, aryl-(C₁₋₈)alkylsulfonyl, heteroarylsulfonyl, heteroaryl-(C₁₋₈)alkylsulfonyl, non-aromatic heterocyclylsulfonyl, (C₃₋₈)cycloalkyl, aryl, aryl-(C₁₋₈)alkyl, heteroaryl, heteroaryl-(C₁₋₈)alkyl or non-aromatic heterocyclyl group G₃, which group G₃ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₄, which group G₄ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₆R₁₇—CR₁₈R₁₉—
wherein R₁₆, R₁₇, R₁₈ and R₁₉ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
   R₁₃ and R₁₄, taken together, are oxo or —CH₂—CH₂—,
for use in the treatment of a disorder or disease selected from the group consisting of insulin resistance, impaired glucose tolerance, type 2 diabetes, obesity, hypertension, and diabetic complications.

### Embodiment 37

Use of a compound according to formula (II):

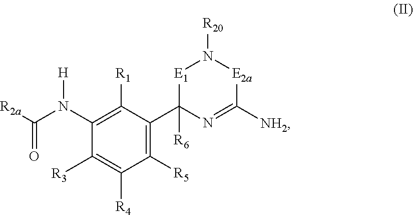

or a pharmaceutically acceptable salt thereof, wherein:
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₂ₐ is an aryl, heteroaryl or non-aromatic heterocyclyl group G₁, which group G₁ is optionally substituted by 1 to 4 substituents independently selected from the group, consisting of cyano, amino, aminocarbonyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
either
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl; and
R₅ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₈)alkylamino-(C₁₋₈)alkyl, N,N-di-[(C₁₋₈)alkyl]amino-(C₁₋₈)alkyl with two identical or different (C₁₋₈)alkyl moieties in the N,N-di-[(C₁₋₈)alkyl]amino moiety, (C₂₋₈)alkenyl, or (C₂₋₈)alkynyl;
R₂₀ is hydrogen, (C₁₋₈)alkyl, (C₁₋₈)alkyl substituted by halogen, (C₃₋₈)cycloalkyl-(C₁₋₈)alkyl, (C₃₋₈)cycloalkoxy-(C₁₋₈)alkyl, aryloxy-(C₁₋₈)alkyl, (C₁₋₈)alkoxy-(C₁₋₈)alkyl, (C₁₋₈)alkylthio-(C₁₋₈)alkyl, (C₁₋₈)alkylsulfinyl, (C₁₋₈)alkylsulfinyl-(C₁₋₈)alkyl, (C₁₋₈)alkylsulfonyl, (C₁₋₈)alkylsulfonyl-(C₁₋₈)alkyl, amino-(C₁₋₈)alkyl, (C₁₋₈)alkylamino-(C₁₋₈)alkyl, di(C₁₋₈)alkylamino-(C₁₋₈)alkyl with two identical or different (C₁₋₈)alkyl moieties in the di(C₁₋₈)alkylamino moiety, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyl-(C₁₋₈)alkyl, (C₁₋₈)alkylcarbonyl-(C₁₋₈)alkyl, (C₁₋₈)alkoxycarbonyl, halogen-(C₁₋₈)alkoxycarbonyl, (C₁₋₈)alkoxycarbonyl-(C₁₋₈)alkyl, (C₁₋₈)alkoxy-(C₁₋₈)alkylcarbonyl, or a (C₃₋₈)cycloalkylcarbonyl, arylcarbonyl, aryl-(C₁₋₈)alkylcarbonyl, heteroarylcarbonyl, heteroaryl-(C₁₋₈)alkylcarbonyl, non-aromatic heterocyclylcarbonyl, (C₃₋₈)cycloalkylsulfonyl, arylsulfonyl, aryl-(C₁₋₈)alkylsulfonyl, heteroarylsulfonyl, heteroaryl-(C₁₋₈)alkylsulfonyl, non-aromatic heterocyclylsulfonyl, (C₃₋₈)cycloalkyl, aryl, aryl-(C₁₋₈)alkyl, heteroaryl, heteroaryl-(C₁₋₈)alkyl or non-aromatic heterocyclyl group G₃, which group G₃ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₄, which group G₄ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₆R₁₇—CR₁₈R₁₉—
wherein R₁₆, R₁₇, R₁₈ and R₁₉ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
   R₁₃ and R₁₄, taken together, are oxo or —CH₂—CH₂—,
in the manufacture of a medicament for the treatment of a disorder or disease selected from the group consisting of insulin resistance, impaired glucose tolerance, type 2 diabetes, obesity, hypertension, and diabetic complications.

### Embodiment 38

A combination comprising a therapeutically effective amount of a compound according to formula (II):

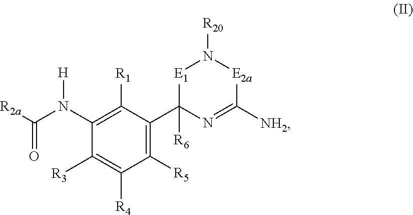

or a pharmaceutically acceptable salt thereof, wherein:
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₂ₐ is an aryl, heteroaryl or non-aromatic heterocyclyl group G₁, which group G₁ is optionally substituted by 1 to 4 substituents independently selected from the group, consisting of cyano, amino, aminocarbonyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
either
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl; and
R₅ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈alkyl]-, —O—, —S—, —S(═O)— or —S(═O)₂—;
R₆ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₈)alkylamino-(C₁₋₈)alkyl, N,N-di-[(C₁₋₈)alkyl]amino-(C₁₋₈)alkyl with two identical or different (C₁₋₈)alkyl moieties in the N,N-di-[(C₁₋₈)alkyl]amino moiety, (C₂₋₈)alkenyl, or (C₂₋₈)alkynyl;
R₂₀ is hydrogen, (C₁₋₈)alkyl, (C₁₋₈)alkyl substituted by halogen, (C₃₋₈)cycloalkyl-(C₁₋₈)alkyl, (C₃₋₈)cycloalkoxy-(C₁₋₈)alkyl, aryloxy-(C₁₋₈)alkyl, (C₁₋₈)alkoxy-(C₁₋₈)alkyl, (C₁₋₈)alkylthio-(C₁₋₈)alkyl, (C₁₋₈)alkylsulfinyl, (C₁₋₈)alkylsulfinyl-(C₁₋₈)alkyl, (C₁₋₈)alkylsulfonyl, (C₁₋₈)alkylsulfonyl-(C₁₋₈)alkyl, amino-(C₁₋₈)alkyl, (C₁₋₈)alkylamino-(C₁₋₈)alkyl, di(C₁₋₈)alkylamino-(C₁₋₈)alkyl with two identical or different (C₁₋₈)alkyl moieties in the di(C₁₋₈)alkylamino moiety, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyl-(C₁₋₈)alkyl, (C₁₋₈)alkylcarbonyl-(C₁₋₈)alkyl, (C₁₋₈)alkoxycarbonyl, halogen-(C₁₋₈)alkoxycarbonyl, (C₁₋₈)alkoxycarbonyl-(C₁₋₈)alkyl, (C₁₋₈)alkoxy-(C₁₋₈)alkylcarbonyl, or a (C₃₋₈)cycloalkylcarbonyl, arylcarbonyl, aryl-(C₁₋₈)alkylcarbonyl, heteroarylcarbonyl, heteroaryl-(C₁₋₈)alkylcarbonyl, non-aromatic heterocyclylcarbonyl, (C₃₋₈)cycloalkylsulfonyl, arylsulfonyl, aryl-(C₁₋₈)alkylsulfonyl, heteroarylsulfonyl, heteroaryl-(C₁₋₈)alkylsulfonyl, non-aromatic heterocyclylsulfonyl, (C₃₋₈)cycloalkyl, aryl, aryl-(C₁₋₈)alkyl, heteroaryl, heteroaryl-(C₁₋₈)alkyl or non-aromatic heterocyclyl group G₃, which group G₃ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₄, which group G₄ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₆R₁₇—CR₁₈R₁₉—
wherein R₁₆, R₁₇, R₁₈ and R₁₉ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
   R₁₃ and R₁₄, taken together, are oxo or —CH₂—CH₂—, and one or more therapeutically active co-agents selected from the group consisting of:
a) antidiabetic agents,
b) hypolipidemic agents,
c) anti-obesity agents,
d) anti-hypertensive agents, and
e) agonists of peroxisome proliferator-activator receptors.

### Embodiment 39

A pharmaceutical compositions comprising:

i) a compound according to formula (II):

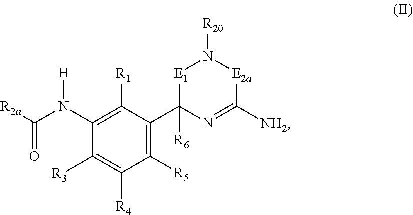

or a pharmaceutically acceptable salt thereof, wherein:
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₂ₐ is an aryl, heteroaryl or non-aromatic heterocyclyl group G₁, which group G₁ is optionally substituted by 1 to 4 substituents independently selected from the group, consisting of cyano, amino, aminocarbonyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
either
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl; and
R₅ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₈)alkylamino-(C₁₋₈)alkyl, N,N-di-[(C₁₋₈)alkyl]amino-(C₁₋₈)alkyl with two identical or different (C₁₋₈)alkyl moieties in the N,N-di-[(C₁₋₈)alkyl]amino moiety, (C₂₋₈)alkenyl, or (C₂₋₈)alkynyl;
R₂₀ is hydrogen, (C₁₋₈)alkyl, (C₁₋₈)alkyl substituted by halogen, (C₃₋₈)cycloalkyl-(C₁₋₈)alkyl, (C₃₋₈)cycloalkoxy-(C₁₋₈)alkyl, aryloxy-(C₁₋₈)alkyl, (C₁₋₈)alkoxy-(C₁₋₈)alkyl, (C₁₋₈)alkylthio-(C₁₋₈)alkyl, (C₁₋₈)alkylsulfinyl, (C₁₋₈)alkylsulfinyl-(C₁₋₈)alkyl, (C₁₋₈)alkylsulfonyl, (C₁₋₈)alkylsulfonyl-(C₁₋₈)alkyl, amino-(C₁₋₈)alkyl, (C₁₋₈)alkylamino-(C₁₋₈)alkyl, di(C₁₋₈)alkylamino-(C₁₋₈)alkyl with two identical or different (C₁₋₈)alkyl moieties in the di(C₁₋₈)alkylamino moiety, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyl-(C₁₋₈)alkyl, (C₁₋₈)alkylcarbonyl-(C₁₋₈)alkyl, (C₁₋₈)alkoxycarbonyl, halogen-(C₁₋₈)alkoxycarbonyl, (C₁₋₈)alkoxycarbonyl-(C₁₋₈)alkyl, (C₁₋₈)alkoxy-(C₁₋₈)alkylcarbonyl, or a (C₃₋₈)cycloalkylcarbonyl, arylcarbonyl, aryl-(C₁₋₈)alkylcarbonyl, heteroarylcarbonyl, heteroaryl-(C₁₋₈)alkylcarbonyl, non-aromatic heterocyclylcarbonyl, (C₃₋₈)cycloalkylsulfonyl, arylsulfonyl, aryl-(C₁₋₈)alkylsulfonyl, heteroarylsulfonyl, heteroaryl-(C₁₋₈)alkylsulfonyl, non-aromatic heterocyclylsulfonyl, (C₃₋₈)cycloalkyl, aryl, aryl-(C₁₋₈)alkyl, heteroaryl, heteroaryl-(C₁₋₈)alkyl or non-aromatic heterocyclyl group G₃, which group G₃ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₄, which group G₄ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₆R₁₇—CR₁₈R₁₉—
wherein R₁₆, R₁₇, R₁₈ and R₁₉ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—, and
   ii) at least one compound selected from
a) antidiabetic agents,
b) hypolipidemic agents,
c) anti-obesity agents,
d) anti-hypertensive agents,
e) agonists of peroxisome proliferator-activator receptors, and
   iii) one or more pharmaceutically acceptable carriers.

### Embodiment 40

A method of modulating BACE-2 activity in a subject, wherein the method comprises administering to the subject a therapeutically effective amount of a compound of formula (III):

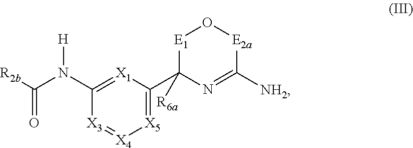

or a pharmaceutically acceptable salt thereof, wherein:
either
X₁ is CR₁ or N;
X₃ is CR₃ or N;
X₄ is CR₄ or N;
X₅ is CR₅ₐ or N;
wherein at least one of X₁, X₃, X₄ and X₅ is N and not more than 2 of X₁, X₃, X₄ and X₅ are N;
or
X₁ is CR₁ or N;
X₃ is CR₃, N or S;
X₄ is a bond;
X₅ is CR₅ₐ, N or S;
wherein at least one of X₁, X₃ and X₅ is N or S, not more than 2 of X₁, X₃ and X₅ are N and not more than 1 of X₃ and X₅ are S;
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R_{2b} is an aryl, heteroaryl or non-aromatic heterocyclyl group G₁, which group G₁ is optionally substituted by 1, 2, 3 or 4 substituents independently selected from the group, consisting of cyano, amino, amino-(C₁₋₈)alkyl, N—(C₁₋₄)alkyl-amino-(C₁₋₈)alkyl, N,N-di(C₁₋₄)alkyl-amino-(C₁₋₈)alkyl, aminocarbonyl, thiocarbamoyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₃₋₈)cycloalkyl-(C₁₋₈)alkoxy, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3, or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₅ₐ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅ₐ, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ₐ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₄)alkyl-amino-(C₁₋₈)alkyl, N,N-di(C₁₋₄)alkyl-amino-(C₁₋₈)alkyl, (C₂₋₈)alkenyl, or (C₂₋₈)alkynyl;
or
R₅ₐ and R₆ₐ, taken together, are a (C₁₋₄)alkylene group, in which (C₁₋₄)alkylene group 1 —CH₂— ring member is optionally replaced with a hetero ring member independently selected from the group, consisting of —N(H)—, —N[(C₁₋₄)alkyl]-, —O—, —S—, —S(═O)— or —S(═O)₂—;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₆R₁₆—CR₁₇R₁₈—
wherein R₁₅, R₁₆, R₁₇ and R₁₈ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—.

### Embodiment 41

A method of treating a disorder or disease associated with inhibition of BACE-2 activity, comprising administering to the subject a therapeutically effective amount of a compound of formula (III):

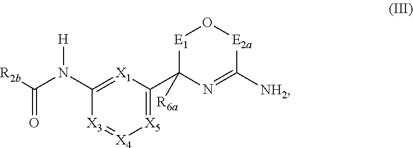

or a pharmaceutically acceptable salt thereof, wherein:
either
X₁ is CR₁ or N;
X₃ is CR₃ or N;
X₄ is CR₄ or N;
X₅ is CR₅ₐ or N;
wherein at least one of X₁, X₃, X₄ and X₅ is N and not more than 2 of X₁, X₃, X₄ and X₅ are N;
or
X₁ is CR₁ or N;
X₃ is CR₃, N or S;
X₄ is a bond;
X₅ is CR₅ₐ, N or S;
wherein at least one of X₁, X₃ and X₅ is N or S, not more than 2 of X₁, X₃ and X₅ are N and not more than 1 of X₃ and X₅ are S;
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R_{2b} is an aryl, heteroaryl or non-aromatic heterocyclyl group G₁, which group G₁ is optionally substituted by 1, 2, 3 or 4 substituents independently selected from the group, consisting of cyano, amino, amino-(C₁₋₈)alkyl, N—(C₁₋₄)alkyl-amino-(C₁₋₈)alkyl, N,N-di(C₁₋₄)alkyl-amino-(C₁₋₈)alkyl, aminocarbonyl, thiocarbamoyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₃₋₈)cycloalkyl-(C₁₋₈)alkoxy, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3, or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₅ₐ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅ₐ taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ₐ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₄)alkyl-amino-(C₁₋₈)alkyl, N,N-di(C₁₋₄)alkyl-amino-(C₁₋₈)alkyl, (C₂₋₈)alkenyl, or (C₂₋₈)alkynyl;
or
R₅ₐ and R₆ₐ, taken together, are a (C₁₋₄)alkylene group, in which (C₁₋₄)alkylene group 1 —CH₂— ring member is optionally replaced with a hetero ring member independently selected from the group, consisting of —N(H)—, —N[(C₁₋₄)alkyl]-, —O—, —S—, —S(═O)— or —S(═O)₂—;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₆R₁₆—CR₁₇R₁₈—
wherein R₁₅, R₁₆, R₁₇ and R₁₈ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
   R₁₃ and R₁₄, taken together, are oxo or —CH₂—CH₂—.

### Embodiment 42

The method according to Embodiment 41 wherein the disorder or disease selected from the group consisting of insulin resistance, impaired glucose tolerance, type 2 diabetes, obesity, hypertension, and diabetic complications.

### Embodiment 43

The method according to Embodiment 42 wherein the disorder or disease selected from the group consisting of impaired glucose tolerance and Type 2 diabetes.

### Embodiment 44

The method according to any one of Embodiments 40 to 43, wherein R₁ is hydrogen.

### Embodiment 45

The method according to any one of Embodiments 40 of 44, wherein R_{2b} is a 5- or 6-membered heteroaryl group in which structure 1, 2, 3, or 4 ring members are hetero ring members independently selected from the group consisting of a nitrogen ring member, an oxygen ring member and a sulfur ring member, which group is optionally substituted by 1, 2, 3 or 4 substituents independently selected from the group, consisting of cyano, amino, aminocarbonyl, thiocarbamoyl, halogen, (C₁₋₄)alkyl, halogen-(C₁₋₄)alkyl, hydroxy, oxo, (C₁₋₄)alkoxy, halogen-(C₁₋₄)alkoxy, (C₁₋₄)alkylthio, halogen-(C₁₋₄)alkylthio, (C₁₋₄)alkoxy-(C₁₋₄)alkyl, (C₁₋₄)alkoxy-(C₁₋₄)alkoxy, (C₁₋₄)alkoxy-(C₁₋₄)alkylthio, (C₁₋₄)alkylthio-(C₁₋₄)alkyl, (C₁₋₄)alkylthio-(C₁₋₄)alkoxy, (C₁₋₄)alkylthio-(C₁₋₄)alkylthio, (C₂₋₄)alkenyl, (C₂₋₄)alkynyl, (C₂₋₄)alkenoxy, and (C₂₋₄)alkynoxy.

### Embodiment 46

The method according to any one of Embodiments 40 to 45, wherein R₃ is hydrogen.

### Embodiment 47

The method according to any one of Embodiments 40 to 46, wherein
X₁ is CH or N;
X₃ is CH or N;
X₄ is CR₄ or N;
X₅ is CR₅;
wherein one and not more than one of X₁, X₃ and X₄ is N.

### Embodiment 48

The method according to any one of Embodiments 40 to 47, wherein R₄ is hydrogen, or halogen; and R₅ₐ is hydrogen, or halogen.

### Embodiment 49

The method according to any one of Embodiments 40 to 48, wherein R₆ₐ is (C₁₋₃)alkyl, or halogen-(C₁₋₃)alkyl.

### Embodiment 50

The method according to any one of Embodiments 40 to 49, wherein E₁ is —C(R₇)(R₈)— and

either
each of R₇ and R₈ is hydrogen;
or
R₇ and R₈, taken together, are oxo.

### Embodiment 51

The method according to any one of Embodiments 40 to 50, wherein E₂ₐ is —C(R₁₁ₐ)(R₁₂ₐ)— and

either
each of R₁₁ₐ and R₁₂ₐ is independently selected from the group, consisting of hydrogen, (C₁₋₃)alkyl and halogen-(C₁₋₃)alkyl;
or
R₁₁ₐ and R₁₂ₐ, taken together, are oxo.

### Embodiment 52

The method according to Embodiment 40, wherein the compound is selected from the group consisting of:
5-Bromo-pyridine-2-carboxylic acid [6-(5-amino-3-methyl-3,6-dihydro-2H-[1,4]-oxazin-3-yl)-pyridin-2-yl]-amide;
5-Chloro-pyridine-2-carboxylic acid [6-(5-amino-3-fluoromethyl-3,6-dihydro-2H-[1,4]oxazin-3-yl)-pyridin-2-yl]-amide;
5-Bromo-pyridine-2-carboxylic acid [6-(5-amino-3-fluoromethyl-3,6-dihydro-2H-[1,4]oxazin-3-yl)-pyridin-2-yl]-amide;
5-Cyano-3-methyl-pyridine-2-carboxylic acid [6-(5-amino-3-fluoromethyl-3,6-dihydro-2H-[1,4]oxazin-3-yl)-pyridin-2-yl]-amide;
4,6-Dideutero-5-chloro-3-trideuteromethyl-pyridine-2-carboxylic acid [6-(5-amino-3-fluoromethyl-3,6-dihydro-2H-[1,4]oxazin-3-yl)-pyridin-2-yl]-amide;
5-Thiocarbamoyl-pyridine-2-carboxylic acid [6-(5-amino-3-fluoromethyl-3,6-dihydro-2H-[1,4]oxazin-3-yl)-pyridin-2-yl]-amide;
5-Cyano-3-methyl-pyridine-2-carboxylic acid [6-(5-amino-3,6-dimethyl-6-trifluoro-methyl-3,6-dihydro-2H-[1,4]oxazin-3-yl)-pyridin-2-yl]-amide;
5-Cyano-pyridine-2-carboxylic acid [6-(5-amino-3,6-dimethyl-6-trifluoromethyl-3,6-dihydro-2H-[1,4]oxazin-3-yl)-pyridin-2-yl]-amide;
5-Cyano-3-methyl-pyridine-2-carboxylic acid [6-(5-amino-3,6-dimethyl-6-trifluoromethyl-3,6-dihydro-2H-[1,4]oxazin-3-yl)-5-fluoro-pyridin-2-yl]-amide;
4,6-Dideutero-5-chloro-3-trideuteromethyl-pyridine-2-carboxylic acid [4-(5-amino-3-fluoromethyl-3,6-dihydro-2H-[1,4]oxazin-3-yl)-pyridin-2-yl]-amide;
5-Chloro-pyridine-2-carboxylic acid [4-(5-amino-3-fluoromethyl-3,6-dihydro-2H-[1,4]oxazin-3-yl)-pyridin-2-yl]-amide;
3-Methyl-5-thiocarbamoyl-pyridine-2-carboxylic acid [4-(5-amino-3-fluoromethyl-3,6-dihydro-2H-[1,4]oxazin-3-yl)-pyridin-2-yl]-amide;
5-Cyano-3-methyl-pyridine-2-carboxylic acid [4-(5-amino-3,6-dimethyl-6-trifluoromethyl-3,6-dihydro-2H-[1,4]oxazin-3-yl)-5-fluoro-pyridin-2-yl]-amide; and
5-Bromo-pyridine-2-carboxylic acid [5-(5-amino-3-fluoromethyl-3,6-dihydro-2H-[1,4]oxazin-3-yl)-6-chloro-pyridin-3-yl]-amide,
   or a pharmaceutically acceptable salt thereof.

### Embodiment 53

A compound according to formula (III):

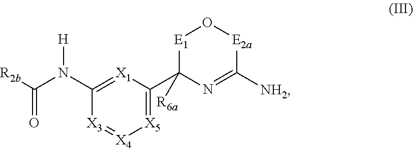

or a pharmaceutically acceptable salt thereof, wherein:
either
X₁ is CR₁ or N;
X₃ is CR₃ or N;
X₄ is CR₄ or N;
X₅ is CR₅ₐ or N;
wherein at least one of X₁, X₃, X₄ and X₅ is N and not more than 2 of X₁, X₃, X₄ and X₅ are N;
or
X₁ is CR₁ or N;
X₃ is CR₃, N or S;
X₄ is a bond;
X₅ is CR₅ₐ, N or S;
wherein at least one of X₁, X₃ and X₅ is N or S, not more than 2 of X₁, X₃ and X₅ are N and not more than 1 of X₃ and X₅ are S;
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R_{2b} is an aryl, heteroaryl or non-aromatic heterocyclyl group G₁, which group G₁ is optionally substituted by 1, 2, 3 or 4 substituents independently selected from the group, consisting of cyano, amino, amino-(C₁₋₈)alkyl, N—(C₁₋₄)alkyl-amino-(C₁₋₈)alkyl, N,N-di(C₁₋₄)alkyl-amino-(C₁₋₈)alkyl, aminocarbonyl, thiocarbamoyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₃₋₈)cycloalkyl-(C₁₋₈)alkoxy, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3, or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₅ₐ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅ₐ taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;

R₆ₐ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₄)alkyl-amino-(C₁₋₈)alkyl, N,N-di(C₁₋₄)alkyl-amino-(C₁₋₈)alkyl, (C₂₋₈)alkenyl, or (C₂₋₈)alkynyl;
or
R₅ₐ and R₆ₐ taken together, are a (C₁₋₄)alkylene group, in which (C₁₋₄)alkylene group 1 —CH₂— ring member is optionally replaced with a hetero ring member independently selected from the group, consisting of —N(H)—, —N[(C₁₋₄)alkyl]-, —O—, —S—, —S(═O)— or —S(═O)₂—;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₅R₁₆—CR₁₇R₁₈—
wherein R₁₅, R₁₆, R₁₇ and R₁₈ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—,
for use in the treatment of a disorder or disease selected from the group consisting of insulin resistance, impaired glucose tolerance, type 2 diabetes, obesity, hypertension, and diabetic complications.

### Embodiment 54

Use of a compound according to formula (III):

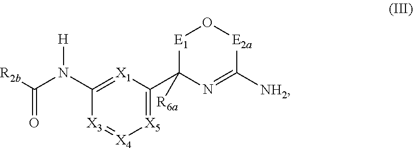

or a pharmaceutically acceptable salt thereof, wherein:
either
X₁ is CR₁ or N;
X₃ is CR₃ or N;
X₄ is CR₄ or N;
X₅ is CR₅ₐ or N;
wherein at least one of X₁, X₃, X₄ and X₅ is N and not more than 2 of X₁, X₃, X₄ and X₅ are N;
or
X₁ is CR₁ or N;
X₃ is CR₃, N or S;
X₄ is a bond;
X₅ is CR₅ₐ, N or S;
wherein at least one of X₁, X₃ and X₅ is N or S, not more than 2 of X₁, X₃ and X₅ are N and not more than 1 of X₃ and X₅ are S;
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R_{2b} is an aryl, heteroaryl or non-aromatic heterocyclyl group G₁, which group G₁ is optionally substituted by 1, 2, 3 or 4 substituents independently selected from the group, consisting of cyano, amino, amino-(C₁₋₈)alkyl, N—(C₁₋₄)alkyl-amino-(C₁₋₈)alkyl, N,N-di(C₁₋₄)alkyl-amino-(C₁₋₈)alkyl, aminocarbonyl, thiocarbamoyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₃₋₈)cycloalkyl-(C₁₋₈)alkoxy, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3, or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₅ₐ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅ₐ, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ₐ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₄)alkyl-amino-(C₁₋₈)alkyl, N,N-di(C₁₋₄)alkyl-amino-(C₁₋₈)alkyl, (C₂₋₈)alkenyl, or (C₂₋₈)alkynyl;
or
R₅ₐ and R₆ₐ, taken together, are a (C₁₋₄)alkylene group, in which (C₁₋₄)alkylene group 1 —CH₂— ring member is optionally replaced with a hetero ring member independently selected from the group, consisting of —N(H)—, —N[(C₁₋₄)alkyl]-, —O—, —S—, —S(═O)— or —S(═O)₂—;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₅R₁₆—CR₁₇R₁₈—
wherein R₁₅, R₁₆, R₁₇ and R₁₈ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—, in the manufacture of a medicament for the treatment of a disorder or disease selected from the group consisting of insulin resistance, impaired glucose tolerance, type 2 diabetes, obesity, hypertension, and diabetic complications.

### Embodiment 55

A combination comprising a therapeutically effective amount of a compound according to formula (III):

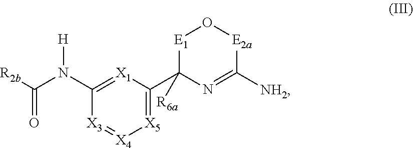

or a pharmaceutically acceptable salt thereof, wherein:
either
X₁ is CR₁ or N;
X₃ is CR₃ or N;
X₄ is CR₄ or N;
X₅ is CR₅ₐ or N;
wherein at least one of X₁, X₃, X₄ and X₅ is N and not more than 2 of X₁, X₃, X₄ and X₅ are N;
or
X₁ is CR₁ or N;
X₃ is CR₃, N or S;
X₄ is a bond;
X₅ is CR₅ₐ, N or S;
wherein at least one of X₁, X₃ and X₅ is N or S, not more than 2 of X₁, X₃ and X₅ are N and not more than 1 of X₃ and X₅ are S;
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R_{2b} is an aryl, heteroaryl or non-aromatic heterocyclyl group G₁, which group G₁ is optionally substituted by 1, 2, 3 or 4 substituents independently selected from the group, consisting of cyano, amino, amino-(C₁₋₈)alkyl, N—(C₁₋₄)alkyl-amino-(C₁₋₈)alkyl, N,N-di(C₁₋₄)alkyl-amino-(C₁₋₈)alkyl, aminocarbonyl, thiocarbamoyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₃₋₈)cycloalkyl-(C₁₋₈)alkoxy, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3, or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₅ₐ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅ₐ, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —[N(C₁₋₈)alkyl]-, —O—, —S—, —S(═O)— or —S(═O)₂—;
R₆ₐ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₄)alkyl-amino-(C₁₋₈)alkyl, N,N-di(C₁₋₄)alkyl-amino-(C₁₋₈)alkyl, (C₂₋₈)alkenyl, or (C₂₋₈)alkynyl;
or
R₅ₐ and R₆ₐ, taken together, are a (C₁₋₄)alkylene group, in which (C₁₋₄)alkylene group 1 —CH₂— ring member is optionally replaced with a hetero ring member independently selected from the group, consisting of —N(H)—, —N[(C₁₋₄)alkyl]-, —O—, —S—, —S(═O)— or —S(═O)₂—;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₅R₁₆—CR₁₇R₁₈—
wherein R₁₅, R₁₆, R₁₇ and R₁₈ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—,
   and one or more therapeutically active co-agents selected from the group consisting of:
a) antidiabetic agents,
b) hypolipidemic agents,
c) anti-obesity agents,
d) anti-hypertensive agents, and
e) agonists of peroxisome proliferator-activator receptors.

### Embodiment 56

A pharmaceutical compositions comprising:

i) a compound according to formula (III):

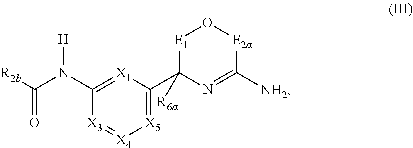

or a pharmaceutically acceptable salt thereof, wherein:
either
X₁ is CR₁ or N;
X₃ is CR₃ or N;
X₄ is CR₄ or N;
X₅ is CR₅ₐ or N;
wherein at least one of X₁, X₃, X₄ and X₅ is N and not more than 2 of X₁, X₃, X₄ and X₅ are N; or
X₁ is CR₁ or N;
X₃ is CR₃, N or S;
X₄ is a bond;
X₅ is CR₅ₐ, N or S;
wherein at least one of X₁, X₃ and X₅ is N or S, not more than 2 of X₁, X₃ and X₅ are N and not more than 1 of X₃ and X₅ are S;
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R_{2b} is an aryl, heteroaryl or non-aromatic heterocyclyl group G₁, which group G₁ is optionally substituted by 1, 2, 3 or 4 substituents independently selected from the group, consisting of cyano, amino, amino-(C₁₋₈)alkyl, N—(C₁₋₄)alkyl-amino-(C₁₋₈)alkyl, N,N-di(C₁₋₄)alkyl-amino-(C₁₋₈)alkyl, aminocarbonyl, thiocarbamoyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₃₋₈)cycloalkyl-(C₁₋₈)alkoxy, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3, or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;

R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₅ₐ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅ₐ, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ₐ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₄)alkyl-amino-(C₁₋₈)alkyl, N,N-di(C₁₋₄)alkyl-amino-(C₁₋₈)alkyl, (C₂₋₈)alkenyl, or (C₂₋₈)alkynyl;
or
R₅ₐ and R₆ₐ, taken together, are a (C₁₋₄)alkylene group, in which (C₁₋₄)alkylene group 1 —CH₂— ring member is optionally replaced with a hetero ring member independently selected from the group, consisting of —N(H)—, —N[(C₁₋₄)alkyl]-, —O—, —S—, —S(═O)— or —S(═O)₂—;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₅R₁₆—CR₁₇R₁₈—
wherein R₁₅, R₁₆, R₁₇ and R₁₈ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—, and
   ii) at least one compound selected from
a) antidiabetic agents,
b) hypolipidemic agents,
c) anti-obesity agents,
d) anti-hypertensive agents,
e) agonists of peroxisome proliferator-activator receptors, and
   iii) one or more pharmaceutically acceptable carriers.

1. A method of modulating BACE-2 activity in a subject, wherein the method comprises administering to the subject a therapeutically effective amount of a compound of formula (I): 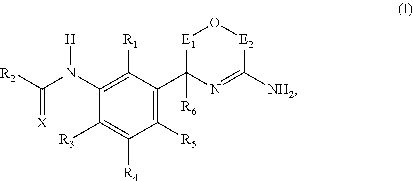
or a pharmaceutically acceptable salt thereof, wherein:
X is O or S;
R₁ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
R₂ is an aryl, heteroaryl or non-aromatic heterocyclyl group G₁, which group G₁ is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3 or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
either
R₄ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl; and
R₅ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]—, —O—, —S—, —S(═O)— or —S(═O)₂—;
R₆ is hydrogen; (C₁₋₈)alkyl; halogen-(C₁₋₈)alkyl; hydroxy-(C₁₋₈)alkyl; (C₁₋₈)alkoxy-(C₁₋₈)alkyl; mercapto-(C₁₋₈)alkyl; (C₁₋₈)alkylthio-(C₁₋₈)alkyl; amino-(C₁₋₈)alkyl; N—(C₁₋₈)alkylamino-(C₁₋₈)alkyl; N,N-di-[(C₁₋₈)alkyl]amino-(C₁₋₈)alkyl with two identical or different (C₁₋₈)alkyl moieties in the N,N-di-[(C₁₋₈)alkyl]amino moiety; (C₂₋₈)alkenyl; or (C₂₋₈)alkynyl;
E₁ is —C(R₇)(R₈)—; or —C(R₇)(R₈)—C(R₉)(R₁₀)—;
E₂ is —C(R₁₁)(R₁₂)—; or —C(R₁₁)(R₁₂)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ and R₁₂ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ and R₁₂, taken together, are oxo or —CH₂—CH₂—; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—.

2. A method of treating a disorder or disease associated with inhibition of BACE-2 activity, comprising administering to the subject a therapeutically effective amount of a compound of formula (I): 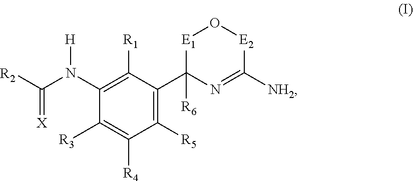
or a pharmaceutically acceptable salt thereof, wherein:
X is O or S;
R₁ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
R₂ is an aryl, heteroaryl or non-aromatic heterocyclyl group G₁, which group G₁ is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3 or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
either
R₄ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl; and
R₅ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—,
R₆ is hydrogen; (C₁₋₈)alkyl; halogen-(C₁₋₈)alkyl; hydroxy-(C₁₋₈)alkyl; (C₁₋₈)alkoxy-(C₁₋₈)alkyl; mercapto-(C₁₋₈)alkyl; (C₁₋₈)alkylthio-(C₁₋₈)alkyl; amino-(C₁₋₈)alkyl; N—(C₁₋₈)alkylamino-(C₁₋₈)alkyl; N,N-di-[(C₁₋₈)alkyl]amino-(C₁₋₈)alkyl with two identical or different (C₁₋₈)alkyl moieties in the N,N-di-[(C₁₋₈)alkyl]amino moiety; (C₂₋₈)alkenyl; or (C₂₋₈)alkynyl;
E₁ is —C(R₇)(R₈)—; or —C(R₇)(R₈)—C(R₉)(R₁₀)—;
E₂ is —C(R₁₁)(R₁₂)—; or —C(R₁₁)(R₁₂)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ and R₁₂ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ and R₁₂, taken together, are oxo or —CH₂—CH₂—; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—.

3. The method according to claim 2 wherein the disorder or disease selected from the group consisting of insulin resistance, impaired glucose tolerance, type 2 diabetes, obesity, hypertension, and diabetic complications.

4. The method according to claim 3 wherein the disorder or disease selected from the group consisting of impaired glucose tolerance and Type 2 diabetes.

5-15. (canceled)

16. The method according to claim 2, wherein the compound is selected from the group consisting of:
Furan-2-carboxylic acid [3-(5-amino-3-methyl-3,6-dihydro-2H-[1,4]oxazin-3-yl)-phenyl]-amide;
5-Bromo-pyridine-2-carboxylic acid [3-(5-amino-3-methyl-3,6-dihydro-2H-[1,4]oxazin-3-yl)-phenyl]-amide;
5-Bromo-pyridine-2-carboxylic acid [3-((R)-5-amino-3-methyl-3,6-di [symbol] hydro-2H-[1,4]oxazin-3-yl)-phenyl]-amide;
5-Bromo-pyridine-2-carboxylic acid [3-((S)-5-amino-3-methyl-3,6-di [symbol] hydro-2H-[1,4]oxazin-3-yl)-phenyl]-amide;
2-Methyl-oxazole-4-carboxylic acid [3-(5-amino-3-methyl-3,6-dihydro-2H-[1,4]oxazin-3-yl)-phenyl]-amide;
5-Methyl-pyrazine-2-carboxylic acid [3-(5-amino-3-methyl-3,6-dihydro-2H-[1,4]oxazin-3-yl)-phenyl]-amide;
5-Bromo-pyrimidine-2-carboxylic acid [3-(5-amino-3-methyl-3,6-dihydro-2H-[1,4]oxazin-3-yl)-phenyl]-amide;
Imidazo[1,2-a]pyridine-2-carboxylic acid [3-(5-amino-3-methyl-3,6-dihydro-2H-[1,4]oxazin-3-yl)-phenyl]-amide;
3-Fluoro-pyridine-2-carboxylic acid [3-(5-amino-3-methyl-3,6-dihydro-2H-[1,4]oxazin-3-yl)-phenyl]-amide;
2,5-Dimethyl-oxazole-4-carboxylic acid [3-(5-amino-3-methyl-3,6-dihydro-2H-[1,4]oxazin-3-yl)-phenyl]-amide;
2-Methyl-thiazole-4-carboxylic acid [3-(5-amino-3-methyl-3,6-dihydro-2H-[1,4]oxazin-3-yl)-phenyl]-amide;
6-Hydroxy-pyridazine-3-carboxylic acid [3-(5-amino-3-methyl-3,6-dihydro-2H-[1,4]oxazin-3-yl)-phenyl]-amide;
Pyridine-2-carboxylic acid [3-(5-amino-3-methyl-3,6-dihydro-2H-[1,4]oxazin-3-yl)-phenyl]-amide;
5-Cyano-pyridine-2-carboxylic acid [3-(5-amino-3-methyl-3,6-dihydro-2H-[1,4]oxazin-3-yl)-phenyl]-amide;
5-(3-Trifluoromethyl-pyrazol-1-yl)-pyrazine-2-carboxylic acid [3-(5-amino-3-methyl-3,6-dihydro-2H-[1,4]oxazin-3-yl)-phenyl]-amide;
5-(3-Methyl-pyrazol-1-yl)-pyrazine-2-carboxylic acid [3-(5-amino-3-methyl-3,6-dihydro-2H-[1,4]oxazin-3-yl)-phenyl]-amide;
3-Chloro-5-trifluoromethyl-pyridine-2-carboxylic acid [3-(5-amino-3-methyl-3,6-dihydro-2H-[1,4]oxazin-3-yl)-phenyl]-amide;
5-Methoxy-pyrazine-2-carboxylic acid [3-(5-amino-3-methyl-3,6-dihydro-2H-[1,4]oxazin-3-yl)-phenyl]-amide;
5-Hydroxy-pyrazine-2-carboxylic acid [3-(5-amino-3-methyl-3,6-dihydro-2H-[1,4]oxazin-3-yl)-phenyl]-amide;
4-Bromo-furan-2-carboxylic acid [3-(5-amino-3-methyl-3,6-dihydro-2H-[1,4]oxazin-3-yl)-phenyl]-amide;
5-Chloro-pyridine-2-carboxylic acid [3-(5-amino-3-methyl-3,6-dihydro-2H-[1,4]oxazin-3-yl)-phenyl]-amide;
5-Trifluoromethyl-pyridine-2-carboxylic acid [3-(5-amino-3-methyl-3,6-dihydro-2H-[1,4]oxazin-3-yl)-phenyl]-amide;
5-Trifluoromethyl-furan-3-carboxylic acid [3-(5-amino-3-methyl-3,6-dihydro-2H-[1,4]oxazin-3-yl)-phenyl]-amide;
N-[3-(5-Amino-3-methyl-3,6-dihydro-2H-[1,4]oxazin-3-yl)-phenyl]-4-bromo-benzamide;
5-Methyl-pyridine-2-carboxylic acid [3-(5-amino-3-methyl-3,6-dihydro-2H-[1,4]oxazin-3-yl)-phenyl]-amide;
N-[3-(5-Amino-3-methyl-3,6-dihydro-2H-[1,4]oxazin-3-yl)-phenyl]-nicotinamide;
5-Bromo-3-methyl-pyridine-2-carboxylic acid [3-((R)-5-amino-3-methyl-3,6-dihydro-2H-[1,4]oxazin-3-yl)-phenyl]-amide;
5-Methoxy-pyridine-2-carboxylic acid [3-((R)-5-amino-3-methyl-3,6-dihydro-2H-[1,4]oxazin-3-yl)-phenyl]-amide;
5-Bromo-pyridine-2-carboxylic acid [3-(5-amino-3-fluoromethyl-3,6-dihydro-2H-[1,4]oxazin-3-yl)-phenyl]-amide;
5-Bromo-pyridine-2-carboxylic acid [3-(5-amino-3-fluoromethyl-3,6-dihydro-2H-[1,4]oxazin-3-yl)-5-chloro-phenyl]-amide;
5-Bromo-pyridine-2-carboxylic acid [3-((R)-5-amino-3-fluoromethyl-3,6-dihydro-2H-[1,4]oxazin-3-yl)-phenyl]-amide;
5-Bromo-pyridine-2-carboxylic acid [3-(5-amino-3-difluoromethyl-3,6-dihydro-2H-[1,4]oxazin-3-yl)-phenyl]-amide;
5-Chloro-pyridine-2-carboxylic acid [3-(5-amino-3-difluoromethyl-3,6-dihydro-2H-[1,4]oxazin-3-yl)-phenyl]-amide;
5-Cyano-pyridine-2-carboxylic acid [3-(5-amino-3-difluoromethyl-3,6-dihydro-2H-[1,4]oxazin-3-yl)-phenyl]-amide;
5-Bromo-3-methyl-pyridine-2-carboxylic acid [3-(5-amino-3-difluoromethyl-3,6-dihydro-2H-[1,4]oxazin-3-yl)-phenyl]-amide;
5-Methyl-pyrazine-2-carboxylic acid [3-(5-amino-3-difluoromethyl-3,6-dihydro-2H-[1,4]oxazin-3-yl)-phenyl]-amide;
5-Trifluoromethyl-pyridine-2-carboxylic acid [3-(5-amino-3-difluoromethyl-3,6-di hydro-2H-[1,4]oxazin-3-yl)-phenyl]-amide;
3-Chloro-5-trifluoromethyl-pyridine-2-carboxylic acid [3-(5-amino-3-difluoromethyl-3,6-dihydro-2H-[1,4]oxazin-3-yl)-phenyl]-amide;
5-Chloro-3-methyl-pyridine-2-carboxylic acid [3-(5-amino-3-difluoromethyl-3,6-dihydro-2H-[1,4]oxazin-3-yl)-phenyl]-amide;
5-Bromo-pyridine-2-carboxylic acid [3-(5-amino-3-difluoromethyl-3,6-dihydro-2H-[1,4]oxazin-3-yl)-4-fluoro-phenyl]-amide;
5-Chloro-pyridine-2-carboxylic acid [3-(5-amino-3-difluoromethyl-3,6-dihydro-2H-[1,4]oxazin-3-yl)-4-fluoro-phenyl]-amide;
5-Chloro-3-methyl-pyridine-2-carboxylic acid [3-((R)-5-amino-3-difluoromethyl-3,6-dihydro-2H-[1,4]oxazin-3-yl)-4-fluoro-phenyl]-amide;
5-Bromo-3-methyl-pyridine-2-carboxylic acid [3-((R)-5-amino-3-difluoromethyl-3,6-dihydro-2H-[1,4]oxazin-3-yl)-4-fluoro-phenyl]-amide;
3-Chloro-5-trifluoromethyl-pyridine-2-carboxylic acid [3-((R)-5-amino-3-difluoromethyl-3,6-dihydro-2H-[1,4]oxazin-3-yl)-4-fluoro-phenyl]-amide;
5-Trifluoromethyl-pyridine-2-carboxylic acid [3-((R)-5-amino-3-difluoromethyl-3,6-dihydro-2H-[1,4]oxazin-3-yl)-4-fluoro-phenyl]-amide;
5-Methyl-pyrazine-2-carboxylic acid [3-((R)-5-amino-3-difluoromethyl-3,6-dihydro-2H-[1,4]oxazin-3-yl)-4-fluoro-phenyl]-amide;
5-Methoxy-pyridine-2-carboxylic acid [3-((R)-5-amino-3-difluoromethyl-3,6-dihydro-2H-[1,4]oxazin-3-yl)-4-fluoro-phenyl]-amide;
5-Chloro-pyridine-2-carboxylic acid [3-(5-amino-3-trifluoromethyl-3,6-dihydro-2H-[1,4]oxazin-3-yl)-4-fluoro-phenyl]-amide;
5-Bromo-pyrimidine-2-carboxylic acid [3-((R)-5-amino-3-methyl-3,6-dihydro-2H-[1,4]oxazin-3-yl)-5-bromo-phenyl]-amide;
5-Bromo-pyridine-2-carboxylic acid [3-((R)-5-amino-3-methyl-3,6-dihydro-2H-[1,4]oxazin-3-yl)-5-bromo-phenyl]-amide;
5-Cyano-pyridine-2-carboxylic acid [3-((R)-5-amino-3-methyl-3,6-dihydro-2H-[1,4]oxazin-3-yl)-5-bromo-phenyl]-amide;
5-Trifluoromethyl-pyridine-2-carboxylic acid [3-((R)-5-amino-3-methyl-3,6-dihydro-2H-[1,4]oxazin-3-yl)-5-bromo-phenyl]-amide;
5-Bromo-3-methyl-pyridine-2-carboxylic acid [3-((R)-5-amino-3-methyl-3,6-dihydro-2H-[1,4]oxazin-3-yl)-5-bromo-phenyl]-amide;
5-Chloro-pyridine-2-carboxylic acid [3-((R)-5-amino-3-methyl-3,6-dihydro-2H-[1,4]oxazin-3-yl)-5-bromo-phenyl]-amide;
5-Methyl-pyrazine-2-carboxylic acid [3-((R)-5-amino-3-methyl-3,6-dihydro-2H-[1,4]oxazin-3-yl)-5-bromo-phenyl]-amide;
2,5-Dimethyl-oxazole-4-carboxylic acid [3-((R)-5-amino-3-methyl-3,6-dihydro-2H-[1,4]oxazin-3-yl)-5-bromo-phenyl]-amide;
2-Methyl-oxazole-4-carboxylic acid [3-((R)-5-amino-3-methyl-3,6-dihydro-2H-[1,4]oxazin-3-yl)-5-bromo-phenyl]-amide;
5-Bromo-pyridine-2-carboxylic acid [3-(5-amino-3-methyl-3,6-dihydro-2H-[1,4]oxazin-3-yl)-4-fluoro-phenyl]-amide;
5-Methyl-pyrazine-2-carboxylic acid [3-(5-amino-3-methyl-3,6-dihydro-2H-[1,4]oxazin-3-yl)-4-fluoro-phenyl]-amide;
5-Cyano-pyridine-2-carboxylic acid [3-(5-amino-3-methyl-3,6-dihydro-2H-[1,4]oxazin-3-yl)-4-fluoro-phenyl]-amide;
5-Chloro-pyridine-2-carboxylic acid [3-(5-amino-3-methyl-3,6-dihydro-2H-[1,4]oxazin-3-yl)-4-fluoro-phenyl]-amide;
5-Bromo-3-methyl-pyridine-2-carboxylic acid [3-(5-amino-3-methyl-3,6-dihydro-2H-[1,4]oxazin-3-yl)-4-fluoro-phenyl]-amide;
Pyridine-2,5-dicarboxylic acid 5-amide 2-{[3-(5-amino-3-methyl-3,6-dihydro-2H-[1,4]oxazin-3-yl)-4-fluoro-phenyl]-amide};
5-Bromo-pyridine-2-carboxylic acid [3-((3R,6R)-5-amino-3,6-dimethyl-6-trifluoromethyl-3,6-dihydro-2H-[1,4]oxazin-3-yl)-phenyl]-amide;
5-Methoxy-pyridine-2-carboxylic acid [3-((3R,6R)-5-amino-3,6-dimethyl-6-trifluoromethyl-3,6-di-hydro-2H-[1,4]oxazin-3-yl)-phenyl]-amide;
5-Bromo-pyridine-2-carboxylic acid [3-((3R,6R)-5-amino-3,6-di-methyl-6-trifluoromethyl-3,6-di-hydro-2H-[1,4]oxazin-3-yl)-4-fluoro-phenyl]-amide;
5-Methoxy-pyridine-2-carboxylic acid [3-((3R,6R)-5-amino-3,6-di-methyl-6-trifluoro [symbol] methyl-3,6-di-hydro-2H-[1,4]oxazin-3-yl)-4-fluoro-phenyl]-amide;
5-Cyano-pyrimidine-2-carboxylic acid [3-((3R,6R)-5-amino-3,6-di-methyl-6-trifluoromethyl-3,6-di-hydro-2H-[1,4]oxazin-3-yl)-4-fluoro-phenyl]-amide;
5-Bromo-pyrimidine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Cyano-3-methyl-pyridine-2-carboxylic acid [3-((3R,6R)-5-amino-3,6-dimethyl-6-trifluoro-methyl-3,6-dihydro-2H-[1,4]oxazin-3-yl)-4-fluoro-phenyl]-amide;
5-Cyano-pyridine-2-carboxylic acid [3-((3R,6R)-5-amino-3,6-dimethyl-6-trifluoro-methyl-3,6-dihydro-2H-[1,4]oxazin-3-yl)-4-fluoro-phenyl]-amide;
5-(2-Methoxy-ethoxy)-pyrazine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
Cyano-pyridine-2-carboxylic acid [3-((3R,6R)-5-amino-3-difluoromethyl-6-methyl-3,6-dihydro-2H-[1,4]oxazin-3-yl)-4-fluoro-phenyl]-amide;
Cyano-pyridine-2-carboxylic acid [3-((3R,6S)-5-amino-3-difluoromethyl-6-methyl-3,6-dihydro-2H-[1,4]oxazin-3-yl)-4-fluoro-phenyl]-amide;
5-Bromo-pyridine-2-carboxylic acid[3-((3R,6R)-5-amino-3-difluoromethyl-6-methyl-3,6-dihydro-2H-[1,4]oxazin-3-yl)-4-fluoro-phenyl]-amide;
5-Bromo-pyridine-2-carboxylic acid[3-((3S,6R)-5-amino-3-difluoromethyl-6-methyl-3,6-dihydro-2H-[1,4]oxazin-3-yl)-4-fluoro-phenyl]-amide;
5-Cyano-pyridine-2-carboxylic acid[3-(5-amino-3-difluoromethyl-6,6-dimethyl-3,6-dihydro-2H-[1,4]oxazin-3-yl)-4-fluoro-phenyl]-amide;
5-Bromo-pyridine-2-carboxylic acid [3-(5-amino-3-difluoromethyl-6,6-dimethyl-3,6-dihydro-2H-[1,4]oxazin-3-yl)-4-fluoro-phenyl]-amide;
5-Bromo-pyridine-2-carboxylic acid [5-(5-amino-3-difluoromethyl-3,6-dihydro-2H-[1,4]oxazin-3-yl)-2,4-difluoro-phenyl]-amide;
5-Cyano-pyridine-2-carboxylic acid [5-(5-amino-3-difluoromethyl-3,6-dihydro-2H-[1,4]oxazin-3-yl)-2,4-difluoro-phenyl]-amide;
5-Bromo-3-methoxy-pyridine-2-carboxylic acid [5-(5-amino-3-difluoromethyl-3,6-dihydro-2H-[1,4]oxazin-3-yl)-2,4-difluoro-phenyl]-amide;
5-Bromo-3-hydroxy-pyridine-2-carboxylic acid [5-(5-amino-3-difluoromethyl-3,6-dihydro-2H-[1,4]oxazin-3-yl)-2,4-difluoro-phenyl]-amide;
5-Chloro-pyridine-2-carboxylic acid [3-((R)-5-amino-3-fluoromethyl-3,6-dihydro-2H-[1,4]oxazin-3-yl)-4-fluoro-phenyl]-amide;
5-Bromo-pyridine-2-carboxylic acid [3-(5-amino-3-fluoromethyl-3,6-dihydro-2H-[1,4]oxazin-3-yl)-4-fluoro-phenyl]-amide;
5-Chloro-pyridine-2-carboxylic acid [3-(5-amino-3-fluoromethyl-3,6-dihydro-2H-[1,4]oxazin-3-yl)-4-fluoro-phenyl]-amide;
3,5-Dichloro-pyridine-2-carboxylic acid [3-(5-amino-3-fluoromethyl-3,6-dihydro-2H-[1,4]oxazin-3-yl)-4-fluoro-phenyl]-amide;
5-Cyano-pyridine-2-carboxylic acid [3-(5-amino-3-fluoromethyl-3,6-dihydro-2H-[1,4]oxazin-3-yl)-4-fluoro-phenyl]-amide;
5-Bromo-3-methyl-pyridine-2-carboxylic acid [3-(5-amino-3-fluoromethyl-3,6-dihydro-2H-[1,4]oxazin-3-yl)-4-fluoro-phenyl]-amide;
5-Bromo-3-methyl-pyridine-2-carboxylic acid [3-((R)-5-amino-3-fluoromethyl-3,6-dihydro-2H-[1,4]oxazin-3-yl)-4-fluoro-phenyl]-amide;
5-Chloro-3-methyl-pyridine-2-carboxylic acid [3-((R)-5-amino-3-fluoromethyl-3,6-dihydro-2H-[1,4]oxazin-3-yl)-4-fluoro-phenyl]-amide;
5-Chloro-4,6-dideutero-3-trideuteromethyl-pyridine-2-carboxylic acid [3-((R)-5-amino-3-fluoromethyl-3,6-dihydro-2H-[1,4]oxazin-3-yl)-4-fluoro-phenyl]-amide;
5-Bromo-3-hydroxy-pyridine-2-carboxylic acid [3-((R)-5-amino-3-fluoromethyl-3,6-dihydro-2H-[1,4]oxazin-3-yl)-4-fluoro-phenyl]-amide;
5-Bromo-3-methoxy-pyridine-2-carboxylic acid [3-((R)-5-amino-3-fluoromethyl-3,6-dihydro-2H-[1,4]oxazin-3-yl)-4-fluoro-phenyl]-amide;
5-Bromo-pyridine-2-carboxylic acid[3-((S)-3-amino-5-difluoromethyl-2,5,6,7-tetrahydro-[1,4]oxazepin-5-yl)-4-fluoro-phenyl]-amide;
5-Bromo-pyridine-2-carboxylic acid[3-((S)-3-amino-5-difluoromethyl-2,5,6,7-tetrahydro-[1,4]oxazepin-5-yl)-4-fluoro-phenyl]-amide;
5-Cyano-pyridine-2-carboxylic acid [3-(3-amino-5-trifluoromethyl-2,5,6,7-tetrahydro-[1,4]oxazepin-5-yl)-4-fluoro-phenyl]-amide;
5-Bromo-pyridine-2-carboxylic acid [3-(3-amino-5-trifluoromethyl-2,5,6,7-tetrahydro-[1,4]oxazepin-5-yl)-4-fluoro-phenyl]-amide;
N-(3-(3-amino-6,6-difluoro-5-methyl-2,5,6,7-tetrahydro-1,4-oxazepin-5-yl)-4-fluorophenyl)-5-chloropicolinamide;
5-Bromo-pyridine-2-carboxylic acid [3-(3-amino-6,6-difluoro-5-methyl-2,5,6,7-tetrahydro-[1,4]oxazepin-5-yl)-4-fluoro-phenyl]-amide;
4-Bromo-furan-2-carboxylic acid [3-(5-amino-3-difluoromethyl-3,6-dihydro-2H-[1,4]oxazin-3-yl)-phenyl]-amide;
6-Hydroxy-pyridazine-3-carboxylic acid [3-(5-amino-3-difluoromethyl-3,6-dihydro-2H-[1,4]oxazin-3-yl)-phenyl]-amide;
2-Methyl-oxazole-4-carboxylic acid [3-(5-amino-3-difluoromethyl-3,6-dihydro-2H-[1,4]oxazin-3-yl)-phenyl]-amide;
2-Ethyl-oxazole-4-carboxylic acid [3-(5-amino-3-difluoromethyl-3,6-dihydro-2H-[1,4]oxazin-3-yl)-phenyl]-amide;
2,5-Dimethyl-oxazole-4-carboxylic acid [3-(5-amino-3-difluoromethyl-3,6-dihydro-2H-[1,4]oxazin-3-yl)-phenyl]-amide;
5-Bromo-3-methoxy-pyridine-2-carboxylic acid [3-(5-amino-3-difluoromethyl-3,6-dihydro-2H-[1,4]oxazin-3-yl)-phenyl]-amide;
5-Bromo-3-hydroxy-pyridine-2-carboxylic acid [3-(5-amino-3-difluoromethyl-3,6-dihydro-2H-[1,4]oxazin-3-yl)-phenyl]-amide;
5-(2-Methoxy-ethoxy)-pyridine-2-carboxylic acid [3-(5-amino-3-difluoromethyl-3,6-dihydro-2H-[1,4]oxazin-3-yl)-phenyl]-amide;
5-Difluoromethyl-pyrazine-2-carboxylic acid [3-(5-amino-3-difluoromethyl-3,6-dihydro-2H-[1,4]oxazin-3-yl)-phenyl]-amide;
3,5-Dichloro-pyridine-2-carboxylic acid [3-((R)-5-amino-3-difluoromethyl-3,6-dihydro-2H-[1,4]oxazin-3-yl)-4-fluoro-phenyl]-amide;
5-Cyano-pyridine-2-carboxylic acid [3-((R)-5-amino-3-difluoromethyl-3,6-dihydro-2H-[1,4]oxazin-3-yl)-4-fluoro-phenyl]-amide;
3,5-Difluoro-pyridine-2-carboxylic acid [3-((R)-5-amino-3-difluoromethyl-3,6-dihydro-2H-[1,4]oxazin-3-yl)-4-fluoro-phenyl]-amide;
5-Bromo-3-methyl-benzofuran-2-carboxylic acid [3-((R)-5-amino-3-difluoromethyl-3,6-dihydro-2H-[1,4]oxazin-3-yl)-4-fluoro-phenyl]-amide;
5-Chloro-4,6-dideutero-3-trideuteromethyl-pyridine-2-carboxylic acid [3-((R)-5-amino-3-difluoromethyl-3,6-dihydro-2H-[1,4]oxazin-3-yl)-4-fluoro-phenyl]-amide;
5-Bromo-4,6-dideutero-3-trideuteromethyl-pyridine-2-carboxylic acid [3-((R)-5-amino-3-difluoromethyl-3,6-dihydro-2H-[1,4]oxazin-3-yl)-4-fluoro-phenyl]-amide;
5-Bromo-3-chloro-pyridine-2-carboxylic acid [3-((R)-5-amino-3-difluoromethyl-3,6-dihydro-2H-[1,4]oxazin-3-yl)-4-fluoro-phenyl]-amide;
5-Bromo-pyridine-2-carboxylic acid [3-((R)-5-amino-3-difluoromethyl-3,6-dihydro-2H-[1,4]oxazin-3-yl)-4-fluoro-phenyl]-amide;
5-Bromo-3-hydroxy-pyridine-2-carboxylic acid [3-((R)-5-amino-3-difluoromethyl-3,6-dihydro-2H-[1,4]oxazin-3-yl)-4-fluoro-phenyl]-amide;
5-Ethoxy-pyridine-2-carboxylic acid [3-((R)-5-amino-3-difluoromethyl-3,6-dihydro-2H-[1,4]oxazin-3-yl)-4-fluoro-phenyl]-amide;
5-Chloro-pyrimidine-2-carboxylic acid [3-((R)-5-amino-3-difluoromethyl-3,6-dihydro-2H-[1,4]oxazin-3-yl)-4-fluoro-phenyl]-amide;
5-Difluoromethyl-pyrazine-2-carboxylic acid [3-((R)-5-amino-3-difluoromethyl-3,6-dihydro-2H-[1,4]oxazin-3-yl)-4-fluoro-phenyl]-amide;
5-(2,2-Difluoro-ethoxy)-pyrazine-2-carboxylic acid [3-((R)-5-amino-3-difluoromethyl-3,6-dihydro-2H-[1,4]oxazin-3-yl)-4-fluoro-phenyl]-amide;
5-(2,2,2-Trifluoro-ethoxy)pyrazine-2-carboxylic acid [3-((R)-5-amino-3-difluoromethyl-3,6-dihydro-2H-[1,4]oxazin-3-yl)-4-fluoro-phenyl]-amide;
4-Bromo-furan-2-carboxylic acid [3-((R)-5-amino-3-difluoromethyl-3,6-dihydro-2H-[1,4]oxazin-3-yl)-4-fluoro-phenyl]-amide;
Pyrazolo[1,5-a]pyridine-2-carboxylic acid [3-((R)-5-amino-3 difluoromethyl-3,6-dihydro-2H [1,4]oxazin-3-yl)-4-fluoro-phenyl]amide;
2-Methyl-oxazole-4-carboxylic acid [3-((R)-5-amino-3-difluoromethyl-3,6-dihydro-2H-[1,4]oxazin-3-yl)-4-fluoro-phenyl]-amide;
2,5-Dimethyl-oxazole-4-carboxylic acid [3-((R)-5-amino-3-difluoromethyl-3,6-dihydro-2H-[1,4]oxazin-3-yl)-4-fluoro-phenyl]-amide;
Imidazo[1,2-a]pyridine-2-carboxylic acid [3-((R)-5-amino-3-difluoromethyl-3,6-dihydro-2H-[1,4]oxazin-3-yl)-4-fluoro-phenyl]-amide;
5-Bromo-3-methoxy-pyridine-2-carboxylic acid [3-((R)-5-amino-3-difluoromethyl-3,6-dihydro-2H-[1,4]oxazin-3-yl)-4-fluoro-phenyl]-amide;
2-Ethyl-oxazole-4-carboxylic acid [3-((R)-5-amino-3-difluoromethyl-3,6-dihydro-2H-[1,4]oxazin-3-yl)-4-fluoro-phenyl]-amide;
5-Difluoromethyl-pyridine-2-carboxylic acid [3-((R)-5-amino-3-difluoromethyl-3,6-dihydro-2H-[1,4]oxazin-3-yl)-4-fluoro-phenyl]-amide;
1-Methyl-1H-imidazole-2 carboxylic acid [3-((R)-5-amino-3 difluoromethyl-3,6-dihydro-2H [1,4]oxazin-3-yl)-4-fluoro-phenyl]amide;
6-Hydroxy-pyridazine-3-carboxylic acid [3-((R)-5-amino-3-difluoromethyl-3,6-dihydro-2H-[1,4]oxazin-3-yl)-4-fluoro-phenyl]-amide;
5-(2-Methoxy-ethoxy)-pyrazine-2-carboxylic acid [3-((R)-5-amino-3-difluoromethyl-3,6-dihydro-2H-[1,4]oxazin-3-yl)-4-fluoro-phenyl]-amide;
5-(2-Fluoro-ethoxy)-pyrazine-2-carboxylic acid [3-((R)-5-amino-3-difluoromethyl-3,6-dihydro-2H-[1,4]oxazin-3-yl)-4-fluoro-phenyl]-amide;
5-Chloro-pyridine-2-carboxylic acid [3-((R)-5-amino-3-difluoromethyl-3,6-dihydro-2H-[1,4]oxazin-3-yl)-4-fluoro-phenyl]-amide;
5-Difluoromethoxy-pyridine-2-carboxylic acid [3-((R)-5-amino-3-difluoromethyl-3,6-dihydro-2H-[1,4]oxazin-3-yl)-4-fluoro-phenyl]-amide;
5-Fluoromethoxy-pyridine-2-carboxylic acid [3-((R)-5-amino-3-difluoromethyl-3,6-dihydro-2H-[1,4]oxazin-3-yl)-4-fluoro-phenyl]-amide;
5-Chloro-3-fluoro-pyridine-2-carboxylic acid [3-((R)-5-amino-3-difluoromethyl-3,6-dihydro-2H-[1,4]oxazin-3-yl)-4-fluoro-phenyl]-amide;
5-Chloro-pyrazine-2-carboxylic acid [3-((R)-5-amino-3-difluoromethyl-3,6-dihydro-2H-[1,4]oxazin-3-yl)-4-fluoro-phenyl]-amide;
5-Methyl-1H-pyrazole-3-carboxylic acid [3-((R)-5-amino-3-difluoromethyl-3,6-dihydro-2H-[1,4]oxazin-3-yl)-4-fluoro-phenyl]-amide;
5-Methyl-pyridine-2-carboxylic acid [3-((R)-5-amino-3-difluoromethyl-3,6-dihydro-2H-[1,4]oxazin-3-yl)-4-fluoro-phenyl]-amide;
5-Cyano-3-methyl-pyridine-2-carboxylic acid [3-((R)-5-amino-3-difluoromethyl-3,6-dihydro-2H-[1,4]oxazin-3-yl)-4-fluoro-phenyl]-amide;
5-Hydroxy-pyrazine-2-carboxylic acid [3-((R)-5-amino-3-difluoromethyl-3,6-dihydro-2H-[1,4]oxazin-3-yl)-4-fluoro-phenyl]-amide;
5-Methoxy-pyrazine-2-carboxylic acid [3-((R)-5-amino-3-difluoromethyl-3,6-dihydro-2H-[1,4]oxazin-3-yl)-4-fluoro-phenyl]-amide;
5-Cyano-4,6-dideutero-3-trideuteromethyl-pyridine-2-carboxylic acid [3-((R)-5-amino-3-difluoromethyl-3,6-dihydro-2H-[1,4]oxazin-3-yl)-4-fluoro-phenyl]-amide;
2-Methyl-thiazole-4-carboxylic acid [3-((R)-5-amino-3-difluoromethyl-3,6-dihydro-2H-[1,4]oxazin-3-yl)-4-fluoro-phenyl]-amide;
5-Methyl-thiazole-2-carboxylic acid [3-((R)-5-amino-3-difluoromethyl-3,6-dihydro-2H-[1,4]oxazin-3-yl)-4-fluoro-phenyl]-amide;
1-Methyl-1H-pyrazole-3-carboxylic acid [3-((R)-5-amino-3-difluoromethyl-3,6-dihydro-2H-[1,4]oxazin-3-yl)-4-fluoro-phenyl]-amide;
1-Methyl-4-nitro-1H-pyrazole-3-carboxylic acid [3-((R)-5-amino-3-difluoromethyl-3,6-dihydro-2H-[1,4]oxazin-3-yl)-4-fluoro-phenyl]-amide;
3-Amino-5-chloro-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Chloro-5-trifluoromethyl-pyridine-2-carboxylic acid [3-(3-amino-6,6-difluoro-5-methyl-2,5,6,7-tetrahydro-[1,4]oxazepin-5-yl)-4-fluoro-phenyl]-amide;
5-Bromo-3-methoxy-pyridine-2-carboxylic acid [3-(3-amino-6,6 difluoro-5-methyl-2,5,6,7 tetrahydro-[1,4]oxazepin-5-yl)-4 fluoro-phenyl]-amide;
5-Cyano-pyridine-2-carboxylic acid [3-(3-amino-6,6-difluoro-5 methyl-2,5,6,7-tetrahydro[1,4]oxazepin-5-yl)-4 fluoro-phenyl]-amide;
7H-Pyrrolo[2,3-d]pyrimidine-6-carboxylic acid [3-((R)-5-amino-3-difluoromethyl-3,6-dihydro-2H-[1,4]oxazin-3-yl)-4-fluoro-phenyl]-amide;
3-Amino-5-(2-methoxy-ethoxy)-pyrazine-2-carboxylic acid [3-((R)-5-amino-3-difluoromethyl-3,6-dihydro-2H-[1,4]oxazin-3-yl)-4-fluoro-phenyl]-amide;
((R)-5-Difluoromethyl-5-{5-[(5-ethyl-pyridine-2-carbonyl)-amino]-2-fluoro-phenyl}-5,6-dihydro-2H-[1,4]oxazin-3-yl)-carbamic acid tert-butyl ester;
3-Amino-5-chloro-pyridine-2-carboxylic acid [3-((R)-5-amino-3-difluoromethyl-3,6-dihydro-2H-[1,4]oxazin-3-yl)-4-fluoro-phenyl]-amide;
3-Chloro-5-methoxy-pyridine-2-carboxylic acid [3-((R)-5-amino-3-difluoromethyl-3,6-dihydro-2H-[1,4]oxazin-3-yl)-4-fluoro-phenyl]-amide;
6-Oxo-1,6-dihydro-pyridine-3-carboxylic acid [3-((R)-5-amino-3-difluoromethyl-3,6-dihydro-2H-[1,4]oxazin-3-yl)-4-fluoro-phenyl]-amide;
Pyrrolo[1,2-c]pyrimidine-3-carboxylic acid [3-((R)-5-amino-3-difluoromethyl-3,6-dihydro-2H-[1,4]oxazin-3-yl)-4-fluoro-phenyl]-amide;
5-But-2-ynyloxy-pyrazine-2-carboxylic acid [3-((R)-5-amino-3-difluoromethyl-3,6-dihydro-2H-[1,4]oxazin-3-yl)-4-fluoro-phenyl]-amide;
3-Amino-5-bromo-pyridine-2-carboxylic acid [3-((R)-5-amino-3-difluoromethyl-3,6-dihydro-2H-[1,4]oxazin-3-yl)-4-fluoro-phenyl]-amide;
1-Ethyl-1H-imidazole-2-carboxylic acid [3-((R)-5-amino-3-difluoromethyl-3,6-dihydro-2H-[1,4]oxazin-3-yl)-4-fluoro-phenyl]-amide;
5-Prop-2-ynyloxy-pyrazine-2-carboxylic acid [3-((R)-5-amino-3-difluoromethyl-3,6-dihydro-2H-[1,4]oxazin-3-yl)-4-fluoro-phenyl]-amide;
5-Amino-2-methyl-oxazole-4-carboxylic acid [3-((R)-5-amino-3-difluoromethyl-3,6-dihydro-2H-[1,4]oxazin-3-yl)-4-fluoro-phenyl]-amide;
5-Chloro-3-hydroxy-pyridine-2-carboxylic acid [3-((R)-5-amino-3-difluoromethyl-3,6-dihydro-2H-[1,4]oxazin-3-yl)-4-fluoro-phenyl]-amide;
5-Isopropoxy-pyrazine-2-carboxylic acid [3-((R)-5-amino-3-difluoromethyl-3,6-dihydro-2H-[1,4]oxazin-3-yl)-4-fluoro-phenyl]-amide;
5-Ethoxy-pyrazine-2-carboxylic acid [3-((R)-5-amino-3-difluoromethyl-3,6-dihydro-2H-[1,4]oxazin-3-yl)-4-fluoro-phenyl]-amide;
5-Dimethylaminomethyl-3-methyl-benzofuran-2-carboxylic acid [3-((R)-5-amino-3-difluoromethyl-3,6-dihydro-2H-[1,4]oxazin-3-yl)-4-fluoro-phenyl]-amide;
1,5-Dimethyl-1H-[1,2,3]triazole-4-carboxylic acid [3-((R)-5-amino-3-difluoromethyl-3,6-dihydro-2H-[1,4]oxazin-3-yl)-4-fluoro-phenyl]-amide;
5-Methoxy-3-methyl-pyrazine-2-carboxylic acid [3-((R)-5-amino-3-difluoromethyl-3,6-dihydro-2H-[1,4]oxazin-3-yl)-4-fluoro-phenyl]-amide;
3-Amino-5-methoxy-pyrazine-2-carboxylic acid [3-((R)-5-amino-3-difluoromethyl-3,6-dihydro-2H-[1,4]oxazin-3-yl)-4-fluoro-phenyl]-amide;
5-Difluoromethoxy-3-methyl-pyridine-2-carboxylic acid [3-((R)-5-amino-3-difluoromethyl-3,6-dihydro-2H-[1,4]oxazin-3-yl)-4-fluoro-phenyl]-amide;
5-Fluoromethoxy-3-methyl-pyridine-2-carboxylic acid [3-((R)-5-amino-3-difluoromethyl-3,6-dihydro-2H-[1,4]oxazin-3-yl)-4-fluoro-phenyl]-amide;
5-(2-Methoxy-ethoxy)-3-methyl-pyrazine-2-carboxylic acid [3-((R)-5-amino-3-difluoromethyl-3,6-dihydro-2H-[1,4]oxazin-3-yl)-4-fluoro-phenyl]-amide;
3,5-Dimethoxy-pyridine-2-carboxylic acid [3-((R)-5-amino-3-difluoromethyl-3,6-dihydro-2H-[1,4]oxazin-3-yl)-4-fluoro-phenyl]-amide;
5-(2-Methoxy-ethoxy)-pyridine-2-carboxylic acid [3-((R)-5-amino-3-difluoromethyl-3,6-dihydro-2H-[1,4]oxazin-3-yl)-4-fluoro-phenyl]-amide;
3-Fluoro-5-(2-methoxy-ethoxy)-pyridine-2-carboxylic acid [3-((R)-5-amino-3-difluoromethyl-3,6-dihydro-2H-[1,4]oxazin-3-yl)-4-fluoro-phenyl]-amide;
5-But-2-ynyloxy-3-methyl-pyrazine-2-carboxylic acid [3-((R)-5-amino-3-difluoromethyl-3,6-dihydro-2H-[1,4]oxazin-3-yl)-4-fluoro-phenyl]-amide;
3-Chloro-5-methoxymethyl-pyridine-2-carboxylic acid [3-((R)-5-amino-3-difluoromethyl-3,6-dihydro-2H-[1,4]oxazin-3-yl)-4-fluoro-phenyl]-amide;
3-Chloro-5-fluoromethoxy-pyridine-2-carboxylic acid [3-((R)-5-amino-3-difluoromethyl-3,6-dihydro-2H-[1,4]oxazin-3-yl)-4-fluoro-phenyl]-amide; and
3-Chloro-5-difluoromethoxy-pyridine-2-carboxylic acid [3-((R)-5-amino-3-difluoromethyl-3,6-dihydro-2H-[1,4]oxazin-3-yl)-4-fluoro-phenyl]-amide,
or a pharmaceutically acceptable salt thereof.

17. The method according to claim 2, wherein the compound is selected from the group consisting of:
5-Chloro-3-methoxymethyl-pyridine-2-carboxylic acid [3-(5-amino-3-difluoromethyl-3,6-dihydro-2H-[1,4]oxazin-3-yl)-4-fluoro-phenyl]-amide;
3-Chloro-5-cyano-pyridine-2-carboxylic acid [3-(5-amino-3-difluoromethyl-3,6-dihydro-2H-[1,4]oxazin-3-yl)-4-fluoro-phenyl]-amide;
3-Amino-5-tris-deutero-methoxy-pyrazine-2-carboxylic acid [3-(5-amino-3-difluoromethyl-3,6-dihydro-2H-[1,4]oxazin-3-yl)-4-fluoro-phenyl]-amide;
3-Amino-5-prop-2-ynyloxy-pyrazine-2-carboxylic acid [3-(5-amino-3-difluoromethyl-3,6-dihydro-2H-[1,4]oxazin-3-yl)-4-fluoro-phenyl]-amide;
3-Chloro-1H-pyrrolo[2,3-b]pyridine-6-carboxylic acid [3-(5-amino-3-difluoromethyl-3,6-dihydro-2H-[1,4]oxazin-3-yl)-4-fluoro-phenyl]-amide;
3-Amino-5-difluoromethyl-pyrazine-2-carboxylic acid [3-(5-amino-3-difluoromethyl-3,6-dihydro-2H-[1,4]oxazin-3-yl)-4-fluoro-phenyl]-amide;
5-Methoxy-3-methyl-pyridine-2-carboxylic acid [3-(5-amino-3-difluoromethyl-3,6-dihydro-2H-[1,4]oxazin-3-yl)-4-fluoro-phenyl]-amide;
5-Difluoromethyl-3-methyl-pyridine-2-carboxylic acid [3-(5-amino-3-difluoromethyl-3,6-dihydro-2H-[1,4]oxazin-3-yl)-4-fluoro-phenyl]-amide;
5-Fluoro-3-trideuteromethoxymethyl-pyridine-2-carboxylic acid [3-(5-amino-3-difluoromethyl-3,6-dihydro-2H-[1,4]oxazin-3-yl)-4-fluoro-phenyl]-amide;
5-Trideuteromethoxy-3-trideuteromethoxymethyl-pyridine-2-carboxylic acid [3-(5-amino-3-difluoromethyl-3,6-dihydro-2H-[1,4]oxazin-3-yl)-4-fluoro-phenyl]-amide;
3-Amino-5-cyano-pyridine-2-carboxylic acid [3-(5-amino-3-difluoromethyl-3,6-dihydro-2H-[1,4]oxazin-3-yl)-4-fluoro-phenyl]-amide;
3-Amino-5-difluoromethyl-pyridine-2-carboxylic acid [3-(5-amino-3-difluoromethyl-3,6-dihydro-2H-[1,4]oxazin-3-yl)-4-fluoro-phenyl]-amide;
5-Trideuteromethoxy-3-methyl-pyridine-2-carboxylic acid [3-(5-amino-3-difluoromethyl-3,6-dihydro-2H-[1,4]oxazin-3-yl)-4-fluoro-phenyl]-amide;
5-Cyano-3-methyl-pyridine-2-carboxylic acid [3-(5-amino-3-difluoromethyl-3,6-dihydro-2H-[1,4]oxazin-3-yl)-4,5-difluoro-phenyl]-amide;
3-Chloro-5-trideuteromethoxy-pyridine-2-carboxylic acid [3-(5-amino-3-difluoromethyl-3,6-dihydro-2H-[1,4]oxazin-3-yl)-4,5-difluoro-phenyl]-amide;
4,6-Dideutero-5-chloro-3-trideuteromethyl-pyridine-2-carboxylic acid [3-(5-amino-3-difluoromethyl-3,6-dihydro-2H-[1,4]oxazin-3-yl)-4,5-difluoro-phenyl]-amide;
3-Chloro-5-cyano-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Chloro-5-methoxy-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Chloro-5-difluoromethoxy-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
5-Chloro-3-methyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
5-Chloro-3-fluoro-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Chloro-5-trideutero-methoxy-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
2,5-Dimethyl-oxazole-4-carboxylic acid [3-(5-amino-3,6-dimethyl-6-trifluoromethyl-3,6-dihydro-2H-[1,4]oxazin-3-yl)-4-fluoro-phenyl]-amide;
5-Difluoromethoxy-3-methyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Amino-5-prop-2-ynyloxy-pyrazine-2-carboxylic acid [3-(5-amino-3,6-dimethyl-6-trifluoromethyl-3,6-dihydro-2H-[1,4]oxazin-3-yl)-4-fluoro-phenyl]-amide;
3-Amino-5-cyano-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
5-Difluoromethyl-3-methyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Amino-5-difluoromethyl-pyrazine-2-carboxylic acid [3-(5-amino-3,6-dimethyl-6-trifluoromethyl-3,6-dihydro-2H-[1,4]oxazin-3-yl)-4-fluoro-phenyl]-amide;
3-Amino-5-difluoromethyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
4-Difluoromethyl-6-methoxy-pyridazine-3-carboxylic acid [3-(5-amino-3,6-dimethyl-6-trifluoromethyl-3,6-dihydro-2H-[1,4]oxazin-3-yl)-4-fluoro-phenyl]-amide;
5-Cyano-3-trideuteromethoxymethyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Chloro-5-difluormethyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
3-Chloro-5-trideuteromethoxy-dideuteromethyl-1H-pyrrolo[2,3-b]pyridine-6-carboxylic acid [3-(5-amino-3,6-dimethyl-6-trifluoromethyl-3,6-dihydro-2H-[1,4]oxazin-3-yl)-4-fluoro-phenyl]-amide;
3-Chloro-5-trifluoromethyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
5-Fluoro-3-methyl-pyridine-2-carboxylic acid [3-(5-amino-3,6-dimethyl-6-trifluoro-methyl-3,6-dihydro-2H-[1,4]oxazin-3-yl)-4-fluoro-phenyl]-amide;
5-Trideuteromethoxy-3-methyl-pyridine-2-carboxylic acid [3-(5-amino-3,6-dimethyl-6-trifluoromethyl-3,6-dihydro-2H-[1,4]oxazin-3-yl)-4-fluoro-phenyl]-amide;
5-Cyano-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
5-Difluoromethoxy-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
5-Chloro-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
5-Cyano-3-methyl-pyridine-2-carboxylic acid [3-(5-amino-3,6,6-tris-fluoromethyl-3,6-dihydro-2H-[1,4]oxazin-3-yl)-4-fluoro-phenyl]-amide;
5-Difluoromethyl-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
3-Amino-5-difluoromethyl-pyrazine-2-carboxylic acid [3-(5-amino-6,6-bis-fluoromethyl-3-methyl-3,6-dihydro-2H-[1,4]oxazin-3-yl)-4-fluoro-phenyl]-amide;
3-Amino-5-difluoromethyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
3-Amino-5-cyano-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
5-Difluoromethyl-3-methyl-pyridine-2-carboxylic acid [3-(5-amino-3,6,6-tris-fluoromethyl-3,6-dihydro-2H-[1,4]oxazin-3-yl)-4-fluoro-phenyl]-amide;
3-Chloro-5-difluoromethyl-pyridine-2-carboxylic acid [3-(5-amino-3,6,6-tris-fluoromethyl-3,6-dihydro-2H-[1,4]oxazin-3-yl)-4-fluoro-phenyl]-amide;
3-Chloro-5-trifluoromethyl-pyridine-2-carboxylic acid [3-(5-amino-3,6,6-tris-fluoromethyl-3,6-dihydro-2H-[1,4]oxazin-3-yl)-4-fluoro-phenyl]-amide;
3,5-Dichloro-pyridine-2-carboxylic acid [3-(5-amino-3,6,6-tris-fluoromethyl-3,6-dihydro-2H-[1,4]oxazin-3-yl)-4-fluoro-phenyl]-amide;
3-Amino-5-cyano-pyridine-2-carboxylic acid [3-(5-amino-3,6,6-tris-fluoromethyl-3,6-dihydro-2H-[1,4]oxazin-3-yl)-4-fluoro-phenyl]-amide;
3-Chloro-5-cyano-pyridine-2-carboxylic acid [3-(5-amino-3,6,6-tris-fluoromethyl-3,6-dihydro-2H-[1,4]oxazin-3-yl)-4-fluoro-phenyl]-amide;
N-[3-(5-Amino-3,6-dimethyl-6-trifluoromethyl-3,6-dihydro-2H-[1,4]oxazin-3-yl)-4-fluoro-phenyl]-6-methoxy-2-methyl-nicotinamide;
N-[3-(5-Amino-3,6-dimethyl-6-trifluoromethyl-3,6-dihydro-2H-[1,4]oxazin-3-yl)-4-fluoro-phenyl]-6-trideuteromethoxy-2-methyl-nicotinamide;
2-Amino-N-[3-(5-amino-3,6-dimethyl-6-trifluoromethyl-3,6-dihydro-2H-[1,4]oxazin-3-yl)-4-fluoro-phenyl]-6-ethoxy-nicotinamide;
2-Amino-N-[3-(5-amino-3,6-dimethyl-6-trifluoromethyl-3,6-dihydro-2H-[1,4]oxazin-3-yl)-4-fluoro-phenyl]-6-methoxy-nicotinamide;
2-Amino-N-[3-(5-amino-3,6-dimethyl-6-trifluoromethyl-3,6-dihydro-2H-[1,4]oxazin-3-yl)-4-fluoro-phenyl]-6-trideuteromethoxy-nicotinamide;
2-Amino-N-[3-(5-amino-3,6-dimethyl-6-trifluoromethyl-3,6-dihydro-2H-[1,4]oxazin-3-yl)-4-fluoro-phenyl]-6-pentadeuteroethoxy-nicotinamide;
N-[3-(5-Amino-3,6-dimethyl-6-trifluoromethyl-3,6-dihydro-2H-[1,4]oxazin-3-yl)-4-fluoro-phenyl]-2-chloro-6-methoxy-nicotinamide;
N-[3-(5-Amino-3,6-dimethyl-6-trifluoromethyl-3,6-dihydro-2H-[1,4]oxazin-3-yl)-4-fluoro-phenyl]-2-chloro-6-ethoxy-nicotinamide;
2-Amino-N-[3-(5-amino-3,6-dimethyl-6-trifluoromethyl-3,6-dihydro-2H-[1,4]oxazin-3-yl)-4-fluoro-phenyl]-6-cyclopropylmethoxy-nicotinamide; and
2-Amino-N-[3-(5-amino-3,6-dimethyl-6-trifluoromethyl-3,6-dihydro-2H-[1,4]oxazin-3-yl)-4-fluoro-phenyl]-6-(2,2,2-trifluoro-ethoxy)-nicotinamide;
or a pharmaceutically acceptable salt thereof.

18. The method according to claim 2, wherein the compound is selected from the group consisting of:
5-Chloro-3-methoxymethyl-pyridine-2-carboxylic acid [3-((R)-5-amino-3-difluoromethyl-3,6-dihydro-2H-[1,4]oxazin-3-yl)-4-fluoro-phenyl]-amide;
3-Chloro-5-cyano-pyridine-2-carboxylic acid [3-((R)-5-amino-3-difluoromethyl-3,6-dihydro-2H-[1,4]oxazin-3-yl)-4-fluoro-phenyl]-amide;
3-Amino-5-tris-deutero-methoxy-pyrazine-2-carboxylic acid [3-((R)-5-amino-3-difluoromethyl-3,6-dihydro-2H-[1,4]oxazin-3-yl)-4-fluoro-phenyl]-amide;
3-Amino-5-prop-2-ynyloxy-pyrazine-2-carboxylic acid [3-((R)-5-amino-3-difluoromethyl-3,6-dihydro-2H-[1,4]oxazin-3-yl)-4-fluoro-phenyl]-amide;
3-Chloro-1H-pyrrolo[2,3-b]pyridine-6-carboxylic acid [3-((R)-5-amino-3-difluoromethyl-3,6-dihydro-2H-[1,4]oxazin-3-yl)-4-fluoro-phenyl]-amide;
3-Amino-5-difluoromethyl-pyrazine-2-carboxylic acid [3-((R)-5-amino-3-difluoromethyl-3,6-dihydro-2H-[1,4]oxazin-3-yl)-4-fluoro-phenyl]-amide;
5-Methoxy-3-methyl-pyridine-2-carboxylic acid [3-((R)-5-amino-3-difluoromethyl-3,6-dihydro-2H-[1,4]oxazin-3-yl)-4-fluoro-phenyl]-amide;
5-Difluoromethyl-3-methyl-pyridine-2-carboxylic acid [3-((R)-5-amino-3-difluoromethyl-3,6-dihydro-2H-[1,4]oxazin-3-yl)-4-fluoro-phenyl]-amide;
5-Fluoro-3-trideuteromethoxymethyl-pyridine-2-carboxylic acid [3-((R)-5-amino-3-difluoromethyl-3,6-dihydro-2H-[1,4]oxazin-3-yl)-4-fluoro-phenyl]-amide;
5-Trideuteromethoxy-3-trideuteromethoxymethyl-pyridine-2-carboxylic acid [3-((R)-5-amino-3-difluoromethyl-3,6-dihydro-2H-[1,4]oxazin-3-yl)-4-fluoro-phenyl]-amide;
3-Amino-5-cyano-pyridine-2-carboxylic acid [3-((R)-5-amino-3-difluoromethyl-3,6-dihydro-2H-[1,4]oxazin-3-yl)-4-fluoro-phenyl]-amide;
3-Amino-5-difluoromethyl-pyridine-2-carboxylic acid [3-((R)-5-amino-3-difluoromethyl-3,6-dihydro-2H-[1,4]oxazin-3-yl)-4-fluoro-phenyl]-amide;
5-Trideuteromethoxy-3-methyl-pyridine-2-carboxylic acid [3-((R)-5-amino-3-difluoromethyl-3,6-dihydro-2H-[1,4]oxazin-3-yl)-4-fluoro-phenyl]-amide;
5-Cyano-3-methyl-pyridine-2-carboxylic acid [3-((R)-5-amino-3-difluoromethyl-3,6-dihydro-2H-[1,4]oxazin-3-yl)-4,5-difluoro-phenyl]-amide;
3-Chloro-5-trideuteromethoxy-pyridine-2-carboxylic acid [3-((R)-5-amino-3-difluoromethyl-3,6-dihydro-2H-[1,4]oxazin-3-yl)-4,5-difluoro-phenyl]-amide;
4,6-Dideutero-5-chloro-3-trideuteromethyl-pyridine-2-carboxylic acid [3-((R)-5-amino-3-difluoromethyl-3,6-dihydro-2H-[1,4]oxazin-3-yl)-4,5-difluoro-phenyl]-amide;
3-Chloro-5-cyano-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Chloro-5-methoxy-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Chloro-5-difluoromethoxy-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Chloro-3-methyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Chloro-3-fluoro-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Chloro-5-trideutero-methoxy-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
2,5-Dimethyl-oxazole-4-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Difluoromethoxy-3-methyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Amino-5-prop-2-ynyloxy-pyrazine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Amino-5-cyano-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Difluoromethyl-3-methyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Amino-5-difluoromethyl-pyrazine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Amino-5-difluoromethyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
4-Difluoromethyl-6-methoxy-pyridazine-3-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Cyano-3-trideuteromethoxymethyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Chloro-5-difluormethyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Chloro-5-trideuteromethoxy-dideuteromethyl-1H-pyrrolo[2,3-b]pyridine-6-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
3-Chloro-5-trifluoromethyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Fluoro-3-methyl-pyridine-2-carboxylic acid [3-((3R,6R)-5-amino-3,6-dimethyl-6-trifluoro-methyl-3,6-dihydro-2H-[1,4]oxazin-3-yl)-4-fluoro-phenyl]-amide;
5-Trideuteromethoxy-3-methyl-pyridine-2-carboxylic acid [3-((3R,6R)-5-amino-3,6-dimethyl-6-trifluoromethyl-3,6-dihydro-2H-[1,4]oxazin-3-yl)-4-fluoro-phenyl]-amide;
5-Cyano-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
5-Cyano-3-methyl-pyridine-2-carboxylic acid [3-((R)-5-amino-6,6-bis-fluoromethyl-3-methyl-3,6-dihydro-2H-[1,4]oxazin-3-yl)-4-fluoro-phenyl]-amide;
5-Difluoromethoxy-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
5-Difluoromethoxy-3-methyl-pyridine-2-carboxylic acid [3-((R)-5-amino-6,6-bis-fluoromethyl-3-methyl-3,6-dihydro-2H-[1,4]oxazin-3-yl)-4-fluoro-phenyl]-amide;
5-Chloro-3-methyl-pyridine-2-carboxylic acid [3-(5-amino-6,6-bis-fluoromethyl-3-methyl-3,6-dihydro-2H-[1,4]oxazin-3-yl)-4-fluoro-phenyl]-amide;
5-Cyano-3-methyl-pyridine-2-carboxylic acid [3-((R)-5-amino-3,6,6-tris-fluoromethyl-3,6-dihydro-2H-[1,4]oxazin-3-yl)-4-fluoro-phenyl]-amide;
5-Difluoromethyl-3-methyl-pyridine-2-carboxylic acid [3-((R)-5-amino-6,6-bis-fluoromethyl-3-methyl-3,6-dihydro-2H-[1,4]oxazin-3-yl)-4-fluoro-phenyl]-amide;
3-Amino-5-difluoromethyl-pyrazine-2-carboxylic acid [3-((R)-5-amino-6,6-bis-fluoromethyl-3-methyl-3,6-dihydro-2H-[1,4]oxazin-3-yl)-4-fluoro-phenyl]-amide;
3-Amino-5-difluoromethyl-pyridine-2-carboxylic acid [3-((R)-5-amino-6,6-bis-fluoromethyl-3-methyl-3,6-dihydro-2H-[1,4]oxazin-3-yl)-4-fluoro-phenyl]-amide;
3-Amino-5-cyano-pyridine-2-carboxylic acid [3-((R)-5-amino-6,6-bis-fluoromethyl-3-methyl-3,6-dihydro-2H-[1,4]oxazin-3-yl)-4-fluoro-phenyl]-amide;
5-Difluoromethyl-3-methyl-pyridine-2-carboxylic acid [3-((R)-5-amino-3,6,6-tris-fluoromethyl-3,6-dihydro-2H-[1,4]oxazin-3-yl)-4-fluoro-phenyl]-amide;
3-Chloro-5-difluoromethyl-pyridine-2-carboxylic acid [3-((R)-5-amino-3,6,6-tris-fluoromethyl-3,6-dihydro-2H-[1,4]oxazin-3-yl)-4-fluoro-phenyl]-amide;
3-Chloro-5-trifluoromethyl-pyridine-2-carboxylic acid [3-((R)-5-amino-3,6,6-tris-fluoromethyl-3,6-dihydro-2H-[1,4]oxazin-3-yl)-4-fluoro-phenyl]-amide;
3,5-Dichloro-pyridine-2-carboxylic acid [3-((R)-5-amino-3,6,6-tris-fluoromethyl-3,6-dihydro-2H-[1,4]oxazin-3-yl)-4-fluoro-phenyl]-amide;
3-Amino-5-cyano-pyridine-2-carboxylic acid [3-((R)-5-amino-3,6,6-tris-fluoromethyl-3,6-dihydro-2H-[1,4]oxazin-3-yl)-4-fluoro-phenyl]-amide;
3-Chloro-5-cyano-pyridine-2-carboxylic acid [3-((R)-5-amino-3,6,6-tris-fluoromethyl-3,6-dihydro-2H-[1,4]oxazin-3-yl)-4-fluoro-phenyl]-amide;
N-[3-((3R,6R)-5-Amino-3,6-dimethyl-6-trifluoromethyl-3,6-dihydro-2H-[1,4]oxazin-3-yl)-4-fluoro-phenyl]-6-methoxy-2-methyl-nicotinamide;
N-[3-((3R,6R)-5-Amino-3,6-dimethyl-6-trifluoromethyl-3,6-dihydro-2H-[1,4]oxazin-3-yl)-4-fluoro-phenyl]-6-trideuteromethoxy-2-methyl-nicotinamide;
2-Amino-N-[3-((3R,6R)-5-amino-3,6-dimethyl-6-trifluoromethyl-3,6-dihydro-2H-[1,4]oxazin-3-yl)-4-fluoro-phenyl]-6-ethoxy-nicotinamide;
2-Amino-N-[3-((3R,6R)-5-amino-3,6-dimethyl-6-trifluoromethyl-3,6-dihydro-2H-[1,4]oxazin-3-yl)-4-fluoro-phenyl]-6-methoxy-nicotinamide;
2-Amino-N-[3-((3R,6R)-5-amino-3,6-dimethyl-6-trifluoromethyl-3,6-dihydro-2H-[1,4]oxazin-3-yl)-4-fluoro-phenyl]-6-trideuteromethoxy-nicotinamide;
2-Amino-N-[3-((3R,6R)-5-amino-3,6-dimethyl-6-trifluoromethyl-3,6-dihydro-2H-[1,4]oxazin-3-yl)-4-fluoro-phenyl]-6-pentadeuteroethoxy-nicotinamide;
N-[3-((3R,6R)-5-Amino-3,6-dimethyl-6-trifluoromethyl-3,6-dihydro-2H-[1,4]oxazin-3-yl)-4-fluoro-phenyl]-2-chloro-6-methoxy-nicotinamide;
N-[3-((3R,6R)-5-Amino-3,6-dimethyl-6-trifluoromethyl-3,6-dihydro-2H-[1,4]oxazin-3-yl)-4-fluoro-phenyl]-2-chloro-6-ethoxy-nicotinamide;
2-Amino-N-[3-((3R,6R)-5-amino-3,6-dimethyl-6-trifluoromethyl-3,6-dihydro-2H-[1,4]oxazin-3-yl)-4-fluoro-phenyl]-6-cyclopropylmethoxy-nicotinamide; and
2-Amino-N-[3-((3R,6R)-5-amino-3,6-dimethyl-6-trifluoromethyl-3,6-dihydro-2H-[1,4]oxazin-3-yl)-4-fluoro-phenyl]-6-(2,2,2-trifluoro-ethoxy)-nicotinamide;
or a pharmaceutically acceptable salt thereof.

19. A compound according to formula (I): 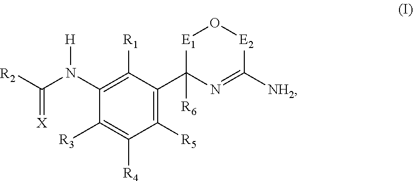
or a pharmaceutically acceptable salt thereof, wherein:
X is O or S;
R₁ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
R₂ is an aryl, heteroaryl or non-aromatic heterocyclyl group G₁, which group G₁ is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3 or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
either
R₄ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl; and
R₅ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—,
R₆ is hydrogen; (C₁₋₈)alkyl; halogen-(C₁₋₈)alkyl; hydroxy-(C₁₋₈)alkyl; (C₁₋₈)alkoxy-(C₁₋₈)alkyl; mercapto-(C₁₋₈)alkyl; (C₁₋₈)alkylthio-(C₁₋₈)alkyl; amino-(C₁₋₈)alkyl; N—(C₁₋₈)alkylamino-(C₁₋₈)alkyl; N,N-di-[(C₁₋₈)alkyl]amino-(C₁₋₈)alkyl with two identical or different (C₁₋₈)alkyl moieties in the N,N-di-[(C₁₋₈)alkyl]amino moiety; (C₂₋₈)alkenyl; or (C₂₋₈)alkynyl;
E₁ is —C(R₇)(R₈)—; or —C(R₇)(R₈)—C(R₉)(R₁₀)—;
E₂ is —C(R₁₁)(R₁₂)—; or —C(R₁₁)(R₁₂)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ and R₁₂ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ and R₁₂, taken together, are oxo or —CH₂—CH₂—; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—,
for use in the treatment of a disorder or disease selected from the group consisting of insulin resistance, impaired glucose tolerance, type 2 diabetes, obesity, hypertension, and diabetic complications.

20. (canceled)

21. A combination comprising a therapeutically effective amount of a compound according to formula (I): 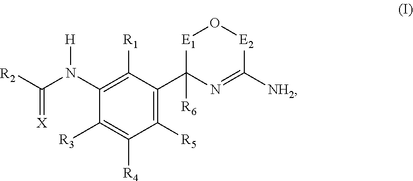
or a pharmaceutically acceptable salt thereof, wherein:
X is O or S;
R₁ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
R₂ is an aryl, heteroaryl or non-aromatic heterocyclyl group G₁, which group G₁ is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3 or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
either
R₄ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl; and
R₅ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—,
R₆ is hydrogen; (C₁₋₈)alkyl; halogen-(C₁₋₈)alkyl; hydroxy-(C₁₋₈)alkyl; (C₁₋₈)alkoxy-(C₁₋₈)alkyl; mercapto-(C₁₋₈)alkyl; (C₁₋₈)alkylthio-(C₁₋₈)alkyl; amino-(C₁₋₈)alkyl; N—(C₁₋₈)alkylamino-(C₁₋₈)alkyl; N,N-di-[(C₁₋₈)alkyl]amino-(C₁₋₈)alkyl with two identical or different (C₁₋₈)alkyl moieties in the N,N-di-[(C₁₋₈)alkyl]amino moiety; (C₂₋₈)alkenyl; or (C₂₋₈)alkynyl;
E₁ is —C(R₇)(R₈)—; or —C(R₇)(R₈)—C(R₉)(R₁₀)—;
E₂ is —C(R₁₁)(R₁₂)—; or —C(R₁₁)(R₁₂)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ and R₁₂ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ and R₁₂, taken together, are oxo or —CH₂—CH₂—; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—, and one or more therapeutically active co-agents selected from the group consisting of:
a) antidiabetic agents,
b) hypolipidemic agents,
c) anti-obesity agents,
d) anti-hypertensive agents, and
e) agonists of peroxisome proliferator-activator receptors.

22. A pharmaceutical compositions comprising:
i) a compound according to formula (I): 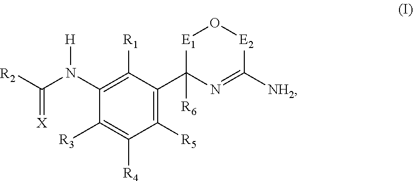
or a pharmaceutically acceptable salt thereof, wherein:
X is O or S;
R₁ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
R₂ is an aryl, heteroaryl or non-aromatic heterocyclyl group G₁, which group G₁ is optionally substituted by 1, 2, 3 or 4 substituents independently selected from the group, consisting of cyano, nitro, amino, aminocarbonyl, amino-(C₁₋₈)alkyl, (C₁₋₄)alkyl-amino-(C₁₋₈)alkyl, di(C₁₋₄)alkyl-amino-(C₁₋₈)alkyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3 or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
either
R₄ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl; and
R₅ is hydrogen; cyano; halogen; (C₁₋₈)alkyl; halogen-(C₁₋₈)alkyl; (C₁₋₈)alkoxy; halogen-(C₁₋₈)alkoxy; (C₁₋₈)alkylthio; halogen-(C₁₋₈)alkylthio; (C₁₋₈)alkoxy-(C₁₋₈)alkyl; (C₁₋₈)alkoxy-(C₁₋₈)alkoxy; (C₁₋₈)alkoxy-(C₁₋₈)alkylthio; (C₁₋₈)alkylthio-(C₁₋₈)alkyl; (C₁₋₈)alkylthio-(C₁₋₈)alkoxy; (C₁₋₈)alkylthio-(C₁₋₈)alkylthio; (C₂₋₈)alkenyl; or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—,
R₆ is hydrogen; (C₁₋₈)alkyl; halogen-(C₁₋₈)alkyl; hydroxy-(C₁₋₈)alkyl; (C₁₋₈)alkoxy-(C₁₋₈)alkyl; mercapto-(C₁₋₈)alkyl; (C₁₋₈)alkylthio-(C₁₋₈)alkyl; amino-(C₁₋₈)alkyl; N—(C₁₋₈)alkylamino-(C₁₋₈)alkyl; N,N-di-[(C₁₋₈)alkyl]amino-(C₁₋₈)alkyl with two identical or different (C₁₋₈)alkyl moieties in the N,N-di-[(C₁₋₈)alkyl]amino moiety; (C₂₋₈)alkenyl; or (C₂₋₈)alkynyl;
E₁ is —C(R₇)(R₈)—; or —C(R₇)(R₈)—C(R₉)(R₁₀)—;
E₂ is —C(R₁₁)(R₁₂)—; or —(R₁₁)(R₁₂)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ and R₁₂ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ and R₁₂, taken together, are oxo or —CH₂—CH₂—; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—, and
ii) at least one compound selected from
a) antidiabetic agents,
b) hypolipidemic agents,
c) anti-obesity agents,
d) anti-hypertensive agents,
e) agonists of peroxisome proliferator-activator receptors, and
iii) one or more pharmaceutically acceptable carriers.

23. A method of modulating BACE-2 activity in a subject, wherein the method comprises administering to the subject a therapeutically effective amount of a compound of formula (II): 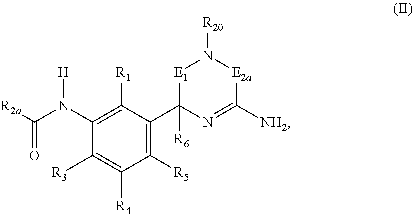
or a pharmaceutically acceptable salt thereof, wherein:
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₂ₐ is an aryl, heteroaryl or non-aromatic heterocyclyl group G₁, which group G₁ is optionally substituted by 1 to 4 substituents independently selected from the group, consisting of cyano, amino, aminocarbonyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
either
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl; and
R₅ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—,
R₆ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₈)alkylamino-(C₁₋₈)alkyl, N,N-di-[(C₁₋₈)alkyl]amino-(C₁₋₈)alkyl with two identical or different (C₁₋₈)alkyl moieties in the N,N-di-[(C₁₋₈)alkyl]amino moiety, (C₂₋₈)alkenyl, or (C₂₋₈)alkynyl;
R₂₀ is hydrogen, (C₁₋₈)alkyl, (C₁₋₈)alkyl substituted by halogen, (C₃₋₈)cycloalkyl-(C₁₋₈)alkyl, (C₃₋₈)cycloalkoxy-(C₁₋₈)alkyl, aryloxy-(C₁₋₈)alkyl, (C₁₋₈)alkoxy-(C₁₋₈)alkyl, (C₁₋₈)alkylthio-(C₁₋₈)alkyl, (C₁₋₈)alkylsulfinyl, (C₁₋₈)alkylsulfinyl-(C₁₋₈)alkyl, (C₁₋₈)alkylsulfonyl, (C₁₋₈)alkylsulfonyl-(C₁₋₈)alkyl, amino-(C₁₋₈)alkyl, (C₁₋₈)alkylamino-(C₁₋₈)alkyl, di(C₁₋₈)alkylamino-(C₁₋₈)alkyl with two identical or different (C₁₋₈)alkyl moieties in the di(C₁₋₈)alkylamino moiety, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyl-(C₁₋₈)alkyl, (C₁₋₈)alkylcarbonyl-(C₁₋₈)alkyl, (C₁₋₈)alkoxycarbonyl, halogen-(C₁₋₈)alkoxycarbonyl, (C₁₋₈)alkoxycarbonyl-(C₁₋₈)alkyl, (C₁₋₈)alkoxy-(C₁₋₈)alkylcarbonyl, or a (C₃₋₈)cycloalkylcarbonyl, arylcarbonyl, aryl-(C₁₋₈)alkylcarbonyl, heteroarylcarbonyl, heteroaryl-(C₁₋₈)alkylcarbonyl, non-aromatic heterocyclylcarbonyl, (C₃₋₈)cycloalkylsulfonyl, arylsulfonyl, aryl-(C₁₋₈)alkylsulfonyl, heteroarylsulfonyl, heteroaryl-(C₁₋₈)alkylsulfonyl, non-aromatic heterocyclyl-sulfonyl, (C₃₋₈)cycloalkyl, aryl, aryl-(C₁₋₈)alkyl, heteroaryl, heteroaryl-(C₁₋₈)alkyl or non-aromatic heterocyclyl group G₃, which group G₃ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₄, which group G₄ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₆R₁₇—CR₁₈R₁₉—
wherein R₁₆, R₁₇, R₁₈ and R₁₉ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—.

24. A method of treating a disorder or disease associated with inhibition of BACE-2 activity, comprising administering to the subject a therapeutically effective amount of a compound of formula (II): 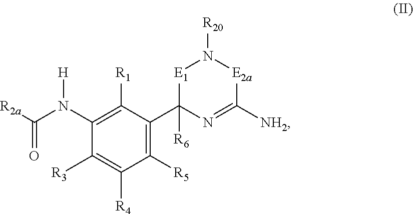
or a pharmaceutically acceptable salt thereof, wherein:
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₂ₐ is an aryl, heteroaryl or non-aromatic heterocyclyl group G₁, which group G₁ is optionally substituted by 1 to 4 substituents independently selected from the group, consisting of cyano, amino, aminocarbonyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
either
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl; and
R₅ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—,
R₆ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₈)alkylamino-(C₁₋₈)alkyl, N,N-di-[(C₁₋₈)alkyl]amino-(C₁₋₈)alkyl with two identical or different (C₁₋₈)alkyl moieties in the N,N-di-[(C₁₋₈)alkyl]amino moiety, (C₂₋₈)alkenyl, or (C₂₋₈)alkynyl;
R₂₀ is hydrogen, (C₁₋₈)alkyl, (C₁₋₈)alkyl substituted by halogen, (C₃₋₈)cycloalkyl-(C₁₋₈)alkyl, (C₃₋₈)cycloalkoxy-(C₁₋₈)alkyl, aryloxy-(C₁₋₈)alkyl, (C₁₋₈)alkoxy-(C₁₋₈)alkyl, (C₁₋₈)alkylthio-(C₁₋₈)alkyl, (C₁₋₈)alkylsulfinyl, (C₁₋₈)alkylsulfinyl-(C₁₋₈)alkyl, (C₁₋₈)alkylsulfonyl, (C₁₋₈)alkylsulfonyl-(C₁₋₈)alkyl, amino-(C₁₋₈)alkyl, (C₁₋₈)alkylamino-(C₁₋₈)alkyl, di(C₁₋₈)alkylamino-(C₁₋₈)alkyl with two identical or different (C₁₋₈)alkyl moieties in the di(C₁₋₈)alkylamino moiety, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyl-(C₁₋₈)alkyl, (C₁₋₈)alkylcarbonyl-(C₁₋₈)alkyl, (C₁₋₈)alkoxycarbonyl, halogen-(C₁₋₈)alkoxycarbonyl, (C₁₋₈)alkoxycarbonyl-(C₁₋₈)alkyl, (C₁₋₈)alkoxy-(C₁₋₈)alkylcarbonyl, or a (C₃₋₈)cycloalkylcarbonyl, arylcarbonyl, aryl-(C₁₋₈)alkylcarbonyl, heteroarylcarbonyl, heteroaryl-(C₁₋₈)alkylcarbonyl, non-aromatic heterocyclylcarbonyl, (C₃₋₈)cycloalkylsulfonyl, arylsulfonyl, aryl-(C₁₋₈)alkylsulfonyl, heteroarylsulfonyl, heteroaryl-(C₁₋₈)alkylsulfonyl, non-aromatic heterocyclyl-sulfonyl, (C₃₋₈)cycloalkyl, aryl, aryl-(C₁₋₈)alkyl, heteroaryl, heteroaryl-(C₁₋₈)alkyl or non-aromatic heterocyclyl group G₃, which group G₃ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₄, which group G₄ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₆R₁₇—CR₁₈R₁₉—
wherein R₁₆, R₁₇, R₁₈ and R₁₉ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—.

25. The method according to claim 24 wherein the disorder or disease selected from the group consisting of insulin resistance, impaired glucose tolerance, type 2 diabetes, obesity, hypertension, and diabetic complications.

26. The method according to claim 25 wherein the disorder or disease selected from the group consisting of impaired glucose tolerance and Type 2 diabetes.

27-34. (canceled)

35. The method according to claim 23, wherein the compound is selected from the group consisting of:
5-Bromo-pyridine-2-carboxylic acid [3-(6-amino-2,4-dimethyl-3-oxo-2,3,4,5-tetrahydro-pyrazin-2-yl)-4-fluoro-phenyl]-amide;
5-Bromo-pyridine-2-carboxylic acid [3-(6-amino-2,4-dimethyl-2,3,4,5-tetrahydro-pyrazin-2-yl)-4-fluoro-phenyl]-amide;
5-Bromo-pyridine-2-carboxylic acid [3-(6-amino-2,4-dimethyl-3-oxo-2,3,4,5-tetrahydro-pyrazin-2-yl)-phenyl]-amide;
5-Bromo-pyridine-2-carboxylic acid [3-(6-amino-2,4-dimethyl-5-oxo-2,3,4,5-tetrahydro-pyrazin-2-yl)-4-fluoro-phenyl]-amide;
5-Chloro-pyridine-2-carboxylic acid [3-(6-amino-2,4-dimethyl-5-oxo-2,3,4,5-tetrahydro-pyrazin-2-yl)-4-fluoro-phenyl]-amide;
5-Bromo-pyridine-2-carboxylic acid [3-(6-amino-4-ethyl-2-methyl-5-oxo-2,3,4,5-tetrahydro-pyrazin-2-yl)-4-fluoro-phenyl]-amide;
3,5-Dichloro-pyridine-2-carboxylic acid [3-(6-amino-2,4-dimethyl-5-oxo-2,3,4,5-tetrahydro-pyrazin-2-yl)-4-fluoro-phenyl]-amide;
3,5-Dichloro-pyridine-2-carboxylic acid [3-(6-amino-4-ethyl-2-methyl-5-oxo-2,3,4,5-tetrahydro-pyrazin-2-yl)-4-fluoro-phenyl]-amide;
5-Bromo-pyridine-2-carboxylic acid [3-(6-amino-4-isopropyl-2-methyl-5-oxo-2,3,4,5-tetrahydro-pyrazin-2-yl)-4-fluoro-phenyl]-amide;
3,5-Dichloro-pyridine-2-carboxylic acid {3-[6-amino-4-(2-methoxy-ethyl)-2-methyl-5-oxo-2,3,4,5-tetrahydro-pyrazin-2-yl]-4-fluoro-phenyl}-amide;
5-Bromo-pyridine-2-carboxylic acid {3-[6-amino-4-(2-methoxy-ethyl)-2-methyl-5-oxo-2,3,4,5-tetrahydro-pyrazin-2-yl]-4-fluoro-phenyl}-amide;
5-Bromo-pyridine-2-carboxylic acid {3-[6-amino-2-methyl-4-(1-methyl-1H-pyrazol-4-yl)-5-oxo-2,3,4,5-tetrahydro-pyrazin-2-yl]-4-fluoro-phenyl}-amide;
5-Bromo-pyridine-2-carboxylic acid [3-(6-amino-2-methyl-5-oxo-4-pyridin-3-yl-2,3,4,5-tetrahydro-pyrazin-2-yl)-4-fluoro-phenyl]-amide;
5-Cyano-pyridine-2-carboxylic acid [3-(6-amino-4-ethyl-2-methyl-5-oxo-2,3,4,5-tetrahydro-pyrazin-2-yl)-4-fluoro-phenyl]-amide;
5-Bromo-pyridine-2-carboxylic acid [3-(6-amino-5-ethyl-2,4-dimethyl-3-oxo-2,3,4,5-tetrahydro-pyrazin-2-yl)-phenyl]-amide;
5-Bromo-pyridine-2-carboxylic acid [3-(6-amino-2-difluoromethyl-2,3,4,5-tetrahydro-pyrazin-2-yl)-4-fluoro-phenyl]-amide;
5-Cyano-pyridine-2-carboxylic acid [3-(6-amino-2-difluoromethyl-2,3,4,5-tetrahydro-pyrazin-2-yl)-4-fluoro-phenyl]-amide;
5-Cyano-pyridine-2-carboxylic acid [3-(6-amino-2-difluoromethyl-5-oxo-2,3,4,5-tetrahydro-pyrazin-2-yl)-4-fluoro-phenyl]-amide;
5-Amino-3-{5-[(5-bromo-pyridine-2-carbonyl)-amino]-2-fluoro-phenyl}-3-difluoromethyl-3,6-dihydro-2H-pyrazine-1-carboxylic acid methyl ester;
5-Amino-3-{5-[(5-cyano-pyridine-2-carbonyl)-amino]-2-fluoro-phenyl}-3-difluoromethyl-3,6-dihydro-2H-pyrazine-1-carboxylic acid methyl ester;
5-Bromo-3-methyl-pyridine-2-carboxylic acid [3-(4-acetyl-6-amino-2-difluoromethyl-2,3,4,5-tetrahydro-pyrazin-2-yl)-4-fluoro-phenyl]-amide;
5-Amino-3-{5-[(5-bromo-3-methyl-pyridine-2-carbonyl)-amino]-2-fluoro-phenyl}-3-difluoromethyl-3,6-dihydro-2H-pyrazine-1-carboxylic acid 2,2-dichloro-ethyl ester;
5-Bromo-3-methyl-pyridine-2-carboxylic acid {3-[6-amino-2-difluoromethyl-4-(2-methoxy-acetyl)-2,3,4,5-tetrahydro-pyrazin-2-yl]-4-fluoro-phenyl}-amide;
5-Bromo-3-methyl-pyridine-2-carboxylic acid [3-(6-amino-4-cyclopropanecarbonyl-2-difluoromethyl-2,3,4,5-tetrahydro-pyrazin-2-yl)-4-fluoro-phenyl]-amide;
5-Bromo-3-methyl-pyridine-2-carboxylic acid [3-(6-amino-2-difluoromethyl-2,3,4,5-tetrahydro-pyrazin-2-yl)-4-fluoro-phenyl]-amide;
5-Difluoromethoxy-3-methyl-pyridine-2-carboxylic acid [3-(4-acetyl-6-amino-2-difluoromethyl-2,3,4,5-tetrahydro-pyrazin-2-yl)-4-fluoro-phenyl]-amide;
5-Difluoromethoxy-3-methyl-pyridine-2-carboxylic acid [3-(6-amino-2-difluoromethyl-2,3,4,5-tetrahydro-pyrazin-2-yl)-4-fluoro-phenyl]-amide;
3-Amino-5-methoxy-pyrazine-2-carboxylic acid [3-(6-amino-2-difluoromethyl-2,3,4,5-tetrahydro-pyrazin-2-yl)-4-fluoro-phenyl]-amide; and
3-Amino-5-oxo-4,5-dihydro-pyrazine-2-carboxylic acid [3-(6-amino-2-difluoromethyl-2,3,4,5-tetrahydro-pyrazin-2-yl)-4-fluoro-phenyl]-amide,
or a pharmaceutically acceptable salt thereof.

36. A compound according to formula (II): 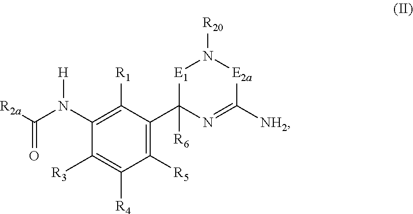
or a pharmaceutically acceptable salt thereof, wherein:
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₂ₐ is an aryl, heteroaryl or non-aromatic heterocyclyl group G₁, which group G₁ is optionally substituted by 1 to 4 substituents independently selected from the group, consisting of cyano, amino, aminocarbonyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
either
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl; and
R₅ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—,
R₆ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₈)alkylamino-(C₁₋₈)alkyl, N,N-di-[(C₁₋₈)alkyl]amino-(C₁₋₈)alkyl with two identical or different (C₁₋₈)alkyl moieties in the N,N-di-[(C₁₋₈)alkyl]amino moiety, (C₂₋₈)alkenyl, or (C₂₋₈)alkynyl;
R₂₀ is hydrogen, (C₁₋₈)alkyl, (C₁₋₈)alkyl substituted by halogen, (C₃₋₈)cycloalkyl-(C₁₋₈)alkyl, (C₃₋₈)cycloalkoxy-(C₁₋₈)alkyl, aryloxy-(C₁₋₈)alkyl, (C₁₋₈)alkoxy-(C₁₋₈)alkyl, (C₁₋₈)alkylthio-(C₁₋₈)alkyl, (C₁₋₈)alkylsulfinyl, (C₁₋₈)alkylsulfinyl-(C₁₋₈)alkyl, (C₁₋₈)alkylsulfonyl, (C₁₋₈)alkylsulfonyl-(C₁₋₈)alkyl, amino-(C₁₋₈)alkyl, (C₁₋₈)alkylamino-(C₁₋₈)alkyl, di(C₁₋₈)alkylamino-(C₁₋₈)alkyl with two identical or different (C₁₋₈)alkyl moieties in the di(C₁₋₈)alkylamino moiety, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyl-(C₁₋₈)alkyl, (C₁₋₈)alkylcarbonyl-(C₁₋₈)alkyl, (C₁₋₈)alkoxycarbonyl, halogen-(C₁₋₈)alkoxycarbonyl, (C₁₋₈)alkoxycarbonyl-(C₁₋₈)alkyl, (C₁₋₈)alkoxy-(C₁₋₈)alkylcarbonyl, or a (C₃₋₈)cycloalkylcarbonyl, arylcarbonyl, aryl-(C₁₋₈)alkylcarbonyl, heteroarylcarbonyl, heteroaryl-(C₁₋₈)alkylcarbonyl, non-aromatic heterocyclylcarbonyl, (C₃₋₈)cycloalkylsulfonyl, arylsulfonyl, aryl-(C₁₋₈)alkylsulfonyl, heteroarylsulfonyl, heteroaryl-(C₁₋₈)alkylsulfonyl, non-aromatic heterocyclyl-sulfonyl, (C₃₋₈)cycloalkyl, aryl, aryl-(C₁₋₈)alkyl, heteroaryl, heteroaryl-(C₁₋₈)alkyl or non-aromatic heterocyclyl group G₃, which group G₃ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₄, which group G₄ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₆R₁₇—CR₁₈R₁₉—
wherein R₁₆, R₁₇, R₁₈ and R₁₉ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—,
for use in the treatment of a disorder or disease selected from the group consisting of insulin resistance, impaired glucose tolerance, type 2 diabetes, obesity, hypertension, and diabetic complications.

37. (canceled)

38. A combination comprising a therapeutically effective amount of a compound according to formula (II): 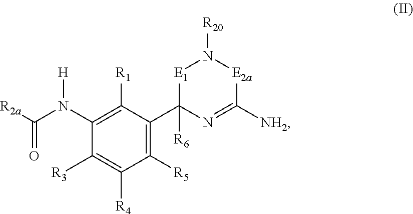
or a pharmaceutically acceptable salt thereof, wherein:
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₂ₐ is an aryl, heteroaryl or non-aromatic heterocyclyl group G₁, which group G₁ is optionally substituted by 1 to 4 substituents independently selected from the group, consisting of cyano, amino, aminocarbonyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
either
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl; and
R₅ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—,
R₆ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₈)alkylamino-(C₁₋₈)alkyl, N,N-di-[(C₁₋₈)alkyl]amino-(C₁₋₈)alkyl with two identical or different (C₁₋₈)alkyl moieties in the N,N-di-[(C₁₋₈)alkyl]amino moiety, (C₂₋₈)alkenyl, or (C₂₋₈)alkynyl;
R₂₀ is hydrogen, (C₁₋₈)alkyl, (C₁₋₈)alkyl substituted by halogen, (C₃₋₈)cycloalkyl-(C₁₋₈)alkyl, (C₃₋₈)cycloalkoxy-(C₁₋₈)alkyl, aryloxy-(C₁₋₈)alkyl, (C₁₋₈)alkoxy-(C₁₋₈)alkyl, (C₁₋₈)alkylthio-(C₁₋₈)alkyl, (C₁₋₈)alkylsulfinyl, (C₁₋₈)alkylsulfinyl-(C₁₋₈)alkyl, (C₁₋₈)alkylsulfonyl, (C₁₋₈)alkylsulfonyl-(C₁₋₈)alkyl, amino-(C₁₋₈)alkyl, (C₁₋₈)alkylamino-(C₁₋₈)alkyl, di(C₁₋₈)alkylamino-(C₁₋₈)alkyl with two identical or different (C₁₋₈)alkyl moieties in the di(C₁₋₈)alkylamino moiety, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyl-(C₁₋₈)alkyl, (C₁₋₈)alkylcarbonyl-(C₁₋₈)alkyl, (C₁₋₈)alkoxycarbonyl, halogen-(C₁₋₈)alkoxycarbonyl, (C₁₋₈)alkoxycarbonyl-(C₁₋₈)alkyl, (C₁₋₈)alkoxy-(C₁₋₈)alkylcarbonyl, or a (C₃₋₈)cycloalkylcarbonyl, arylcarbonyl, aryl-(C₁₋₈)alkylcarbonyl, heteroarylcarbonyl, heteroaryl-(C₁₋₈)alkylcarbonyl, non-aromatic heterocyclylcarbonyl, (C₃₋₈)cycloalkylsulfonyl, arylsulfonyl, aryl-(C₁₋₈)alkylsulfonyl, heteroarylsulfonyl, heteroaryl-(C₁₋₈)alkylsulfonyl, non-aromatic heterocyclyl-sulfonyl, (C₃₋₈)cycloalkyl, aryl, aryl-(C₁₋₈)alkyl, heteroaryl, heteroaryl-(C₁₋₈)alkyl or non-aromatic heterocyclyl group G₃, which group G₃ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₄, which group G₄ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₆R₁₇—CR₁₈R₁₉—
wherein R₁₆, R₁₇, R₁₈ and R₁₉ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—, and one or more therapeutically active co-agents selected from the group consisting of:
a) antidiabetic agents,
b) hypolipidemic agents,
c) anti-obesity agents,
d) anti-hypertensive agents, and
e) agonists of peroxisome proliferator-activator receptors.

39. A pharmaceutical compositions comprising:
i) a compound according to formula (II): 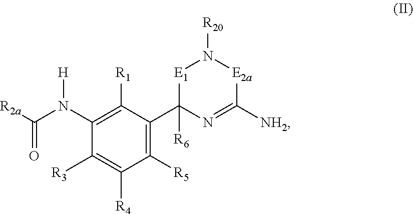
or a pharmaceutically acceptable salt thereof, wherein:
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₂ₐ is an aryl, heteroaryl or non-aromatic heterocyclyl group G₁, which group G₁ is optionally substituted by 1 to 4 substituents independently selected from the group, consisting of cyano, amino, aminocarbonyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
either
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl; and
R₅ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—,
R₆ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₈)alkylamino-(C₁₋₈)alkyl, N,N-di-[(C₁₋₈)alkyl]amino-(C₁₋₈)alkyl with two identical or different (C₁₋₈)alkyl moieties in the N,N-di-[(C₁₋₈)alkyl]amino moiety, (C₂₋₈)alkenyl, or (C₂₋₈)alkynyl;
R₂₀ is hydrogen, (C₁₋₈)alkyl, (C₁₋₈)alkyl substituted by halogen, (C₃₋₈)cycloalkyl-(C₁₋₈)alkyl, (C₃₋₈)cycloalkoxy-(C₁₋₈)alkyl, aryloxy-(C₁₋₈)alkyl, (C₁₋₈)alkoxy-(C₁₋₈)alkyl, (C₁₋₈)alkylthio-(C₁₋₈)alkyl, (C₁₋₈)alkylsulfinyl, (C₁₋₈)alkylsulfinyl-(C₁₋₈)alkyl, (C₁₋₈)alkylsulfonyl, (C₁₋₈)alkylsulfonyl-(C₁₋₈)alkyl, amino-(C₁₋₈)alkyl, (C₁₋₈)alkylamino-(C₁₋₈)alkyl, di(C₁₋₈)alkylamino-(C₁₋₈)alkyl with two identical or different (C₁₋₈)alkyl moieties in the di(C₁₋₈)alkylamino moiety, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyl-(C₁₋₈)alkyl, (C₁₋₈)alkylcarbonyl-(C₁₋₈)alkyl, (C₁₋₈)alkoxycarbonyl, halogen-(C₁₋₈)alkoxycarbonyl, (C₁₋₈)alkoxycarbonyl-(C₁₋₈)alkyl, (C₁₋₈)alkoxy-(C₁₋₈)alkylcarbonyl, or a (C₃₋₈)cycloalkylcarbonyl, arylcarbonyl, aryl-(C₁₋₈)alkylcarbonyl, heteroarylcarbonyl, heteroaryl-(C₁₋₈)alkylcarbonyl, non-aromatic heterocyclylcarbonyl, (C₃₋₈)cycloalkylsulfonyl, arylsulfonyl, aryl-(C₁₋₈)alkylsulfonyl, heteroarylsulfonyl, heteroaryl-(C₁₋₈)alkylsulfonyl, non-aromatic heterocyclyl-sulfonyl, (C₃₋₈)cycloalkyl, aryl, aryl-(C₁₋₈)alkyl, heteroaryl, heteroaryl-(C₁₋₈)alkyl or non-aromatic heterocyclyl group G₃, which group G₃ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl and a (C₃₋₈)cycloalkyl, aryl, heteroaryl or non-aromatic heterocyclyl group G₄, which group G₄ is optionally substituted by 1 to 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₆R₁₇—CR₁₈R₁₉—
wherein R₁₆, R₁₇, R₁₈ and R₁₉ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—, and
ii) at least one compound selected from
a) antidiabetic agents,
b) hypolipidemic agents,
c) anti-obesity agents,
d) anti-hypertensive agents,
e) agonists of peroxisome proliferator-activator receptors, and
iii) one or more pharmaceutically acceptable carriers.

40. A method of modulating BACE-2 activity in a subject, wherein the method comprises administering to the subject a therapeutically effective amount of a compound of formula (III): 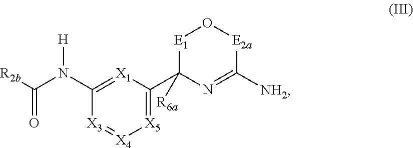
or a pharmaceutically acceptable salt thereof, wherein:
either
X₁ is CR₁ or N;
X₃ is CR₃ or N;
X₄ is CR₄ or N;
X₅ is CR₅ₐ or N;
wherein at least one of X₁, X₃, X₄ and X₅ is N and not more than 2 of X₁, X₃, X₄ and X₅ are N;
or
X₁ is CR₁ or N;
X₃ is CR₃, N or S;
X₄ is a bond;
X₅ is CR₅ₐ, N or S;
wherein at least one of X₁, X₃ and X₅ is N or S, not more than 2 of X₁, X₃ and X₅ are N and not more than 1 of X₃ and X₅ are S;
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R_{2b} is an aryl, heteroaryl or non-aromatic heterocyclyl group G₁, which group G₁ is optionally substituted by 1, 2, 3 or 4 substituents independently selected from the group, consisting of cyano, amino, amino-(C₁₋₈)alkyl, N—(C₁₋₄)alkyl-amino-(C₁₋₈)alkyl, N,N-di(C₁₋₄)alkyl-amino-(C₁₋₈)alkyl, aminocarbonyl, thiocarbamoyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₃₋₈)cycloalkyl-(C₁₋₈)alkoxy, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3, or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₅ₐ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅ₐ, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ₐ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₄)alkyl-amino-(C₁₋₈)alkyl, N,N-di(C₁₋₄)alkyl-amino-(C₁₋₈)alkyl, (C₂₋₈)alkenyl, or (C₂₋₈)alkynyl;
or
R₅ₐ and R₆ₐ, taken together, are a (C₁₋₄)alkylene group, in which (C₁₋₄)alkylene group 1 —CH₂— ring member is optionally replaced with a hetero ring member independently selected from the group, consisting of —N(H)—, —N[(C₁₋₄)alkyl]-, —O—, —S—, —S(═O)— or —S(═O)₂—;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₅R₁₆—CR₁₇R₁₈—
wherein R₁₅, R₁₆, R₁₇ and R₁₈ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—.

41. A method of treating a disorder or disease associated with inhibition of BACE-2 activity, comprising administering to the subject a therapeutically effective amount of a compound of formula (III): 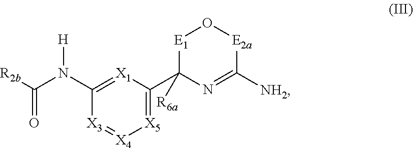
or a pharmaceutically acceptable salt thereof, wherein:
either
X₁ is CR₁ or N;
X₃ is CR₃ or N;
X₄ is CR₄ or N;
X₅ is CR₅ₐ or N;
wherein at least one of X₁, X₃, X₄ and X₅ is N and not more than 2 of X₁, X₃, X₄ and X₅ are N;
or
X₁ is CR₁ or N;
X₃ is CR₃, N or S;
X₄ is a bond;
X₅ is CR₅ₐ, N or S;
wherein at least one of X₁, X₃ and X₅ is N or S, not more than 2 of X₁, X₃ and X₅ are N and not more than 1 of X₃ and X₅ are S;
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R_{2b} is an aryl, heteroaryl or non-aromatic heterocyclyl group G₁, which group G₁ is optionally substituted by 1, 2, 3 or 4 substituents independently selected from the group, consisting of cyano, amino, amino-(C₁₋₈)alkyl, N—(C₁₋₄)alkyl-amino-(C₁₋₈)alkyl, N,N-di(C₁₋₄)alkyl-amino-(C₁₋₈)alkyl, aminocarbonyl, thiocarbamoyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₃₋₈)cycloalkyl-(C₁₋₈)alkoxy, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3, or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₅ₐ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅ₐ, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ₐ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₄)alkyl-amino-(C₁₋₈)alkyl, N,N-di(C₁₋₄)alkyl-amino-(C₁₋₈)alkyl, (C₂₋₈)alkenyl, or (C₂₋₈)alkynyl;
or
R₅ₐ and R₆ₐ, taken together, are a (C₁₋₄)alkylene group, in which (C₁₋₄)alkylene group 1 —CH₂— ring member is optionally replaced with a hetero ring member independently selected from the group, consisting of —N(H)—, —N[(C₁₋₄)alkyl]-, —O—, —S—, —S(═O)— or —S(═O)₂—;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₅R₁₆—CR₁₇R₁₈—
wherein R₁₅, R₁₆, R₁₇ and R₁₈ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—.

42. The method according to claim 41 wherein the disorder or disease selected from the group consisting of insulin resistance, impaired glucose tolerance, type 2 diabetes, obesity, hypertension, and diabetic complications.

43. The method according to claim 42 wherein the disorder or disease selected from the group consisting of impaired glucose tolerance and Type 2 diabetes.

44-51. (canceled)

52. The method according to claim 40, wherein the compound is selected from the group consisting of:
5-Bromo-pyridine-2-carboxylic acid [6-(5-amino-3-methyl-3,6-dihydro-2H-[1,4]-oxazin-3-yl)-pyridin-2-yl]-amide;
5-Chloro-pyridine-2-carboxylic acid [6-(5-amino-3-fluoromethyl-3,6-dihydro-2H-[1,4]oxazin-3-yl)-pyridin-2-yl]-amide;
5-Bromo-pyridine-2-carboxylic acid [6-(5-amino-3-fluoromethyl-3,6-dihydro-2H-[1,4]oxazin-3-yl)-pyridin-2-yl]-amide;
5-Cyano-3-methyl-pyridine-2-carboxylic acid [6-(5-amino-3-fluoromethyl-3,6-dihydro-2H-[1,4]oxazin-3-yl)-pyridin-2-yl]-amide;
4,6-Dideutero-5-chloro-3-trideuteromethyl-pyridine-2-carboxylic acid [6-(5-amino-3-fluoromethyl-3,6-dihydro-2H-[1,4]oxazin-3-yl)-pyridin-2-yl]-amide;
5-Thiocarbamoyl-pyridine-2-carboxylic acid [6-(5-amino-3-fluoromethyl-3,6-dihydro-2H-[1,4]oxazin-3-yl)-pyridin-2-yl]-amide;
5-Cyano-3-methyl-pyridine-2-carboxylic acid [6-(5-amino-3,6-dimethyl-6-trifluoro-methyl-3,6-dihydro-2H-[1,4]oxazin-3-yl)-pyridin-2-yl]-amide;
5-Cyano-pyridine-2-carboxylic acid [6-(5-amino-3,6-dimethyl-6-trifluoromethyl-3,6-dihydro-2H-[1,4]oxazin-3-yl)-pyridin-2-yl]-amide;
5-Cyano-3-methyl-pyridine-2-carboxylic acid [6-(5-amino-3,6-dimethyl-6-trifluoromethyl-3,6-dihydro-2H-[1,4]oxazin-3-yl)-5-fluoro-pyridin-2-yl]-amide;
4,6-Dideutero-5-chloro-3-trideuteromethyl-pyridine-2-carboxylic acid [4-(5-amino-3-fluoromethyl-3,6-dihydro-2H-[1,4]oxazin-3-yl)-pyridin-2-yl]-amide;
5-Chloro-pyridine-2-carboxylic acid [4-(5-amino-3-fluoromethyl-3,6-dihydro-2H-[1,4]oxazin-3-yl)-pyridin-2-yl]-amide;
3-Methyl-5-thiocarbamoyl-pyridine-2-carboxylic acid [4-(5-amino-3-fluoromethyl-3,6-dihydro-2H-[1,4]oxazin-3-yl)-pyridin-2-yl]-amide;
5-Cyano-3-methyl-pyridine-2-carboxylic acid [4-(5-amino-3,6-dimethyl-6-trifluoromethyl-3,6-dihydro-2H-[1,4]oxazin-3-yl)-5-fluoro-pyridin-2-yl]-amide; and
5-Bromo-pyridine-2-carboxylic acid [5-(5-amino-3-fluoromethyl-3,6-dihydro-2H-[1,4]oxazin-3-yl)-6-chloro-pyridin-3-yl]-amide,
or a pharmaceutically acceptable salt thereof.

53. A compound according to formula (III): 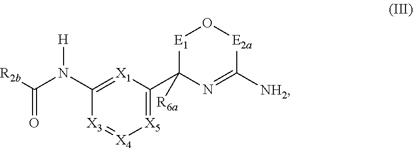
or a pharmaceutically acceptable salt thereof, wherein:
either
X₁ is CR₁ or N;
X₃ is CR₃ or N;
X₄ is CR₄ or N;
X₅ is CR₅ₐ or N;
wherein at least one of X₁, X₃, X₄ and X₅ is N and not more than 2 of X₁, X₃, X₄ and X₅ are N;
or
X₁ is CR₁ or N;
X₃ is CR₃, N or S;
X₄ is a bond;
X₅ is CR₅ₐ, N or S;
wherein at least one of X₁, X₃ and X₅ is N or S, not more than 2 of X₁, X₃ and X₅ are N and not more than 1 of X₃ and X₅ are S;
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R_{2b} is an aryl, heteroaryl or non-aromatic heterocyclyl group G₁, which group G₁ is optionally substituted by 1, 2, 3 or 4 substituents independently selected from the group, consisting of cyano, amino, amino-(C₁₋₈)alkyl, N—(C₁₋₄)alkyl-amino-(C₁₋₈)alkyl, N,N-di(C₁₋₄)alkyl-amino-(C₁₋₈)alkyl, aminocarbonyl, thiocarbamoyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₃₋₈)cycloalkyl-(C₁₋₈)alkoxy, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3, or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₅ₐ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅ₐ, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ₐ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₄)alkyl-amino-(C₁₋₈)alkyl, N,N-di(C₁₋₄)alkyl-amino-(C₁₋₈)alkyl, (C₂₋₈)alkenyl, or (C₂₋₈)alkynyl;
or
R₅ₐ and R₆ₐ, taken together, are a (C₁₋₄)alkylene group, in which (C₁₋₄)alkylene group 1 —CH₂— ring member is optionally replaced with a hetero ring member independently selected from the group, consisting of —N(H)—, —N[(C₁₋₄)alkyl]-, —O—, —S—, —S(═O)— or —S(═O)₂—;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₅R₁₆—CR₁₇R₁₈—
wherein R₁₅, R₁₆, R₁₇ and R₁₈ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—,
for use in the treatment of a disorder or disease selected from the group consisting of insulin resistance, impaired glucose tolerance, type 2 diabetes, obesity, hypertension, and diabetic complications.

54. (canceled)

55. A combination comprising a therapeutically effective amount of a compound according to formula (III): 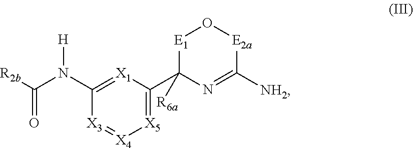
or a pharmaceutically acceptable salt thereof, wherein:
either
X₁ is CR₁ or N;
X₃ is CR₃ or N;
X₄ is CR₄ or N;
X₅ is CR₅ₐ or N;
wherein at least one of X₁, X₃, X₄ and X₅ is N and not more than 2 of X₁, X₃, X₄ and X₅ are N;
or
X₁ is CR₁ or N;
X₃ is CR₃, N or S;
X₄ is a bond;
X₅ is CR₅ₐ, N or S;
wherein at least one of X₁, X₃ and X₅ is N or S, not more than 2 of X₁, X₃ and X₅ are N and not more than 1 of X₃ and X₅ are S;
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R_{2b} is an aryl, heteroaryl or non-aromatic heterocyclyl group G₁, which group G₁ is optionally substituted by 1, 2, 3 or 4 substituents independently selected from the group, consisting of cyano, amino, amino-(C₁₋₈)alkyl, N—(C₁₋₄)alkyl-amino-(C₁₋₈)alkyl, N,N-di(C₁₋₄)alkyl-amino-(C₁₋₈)alkyl, aminocarbonyl, thiocarbamoyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₃₋₈)cycloalkyl-(C₁₋₈)alkoxy, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3, or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₅ₐ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅ₐ, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ₐ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₄)alkyl-amino-(C₁₋₈)alkyl, N,N-di(C₁₋₄)alkyl-amino-(C₁₋₈)alkyl, (C₂₋₈)alkenyl, or (C₂₋₈)alkynyl;
or
R₅ₐ and R₆ₐ, taken together, are a (C₁₋₄)alkylene group, in which (C₁₋₄)alkylene group 1 —CH₂— ring member is optionally replaced with a hetero ring member independently selected from the group, consisting of —N(H)—, —N[(C₁₋₄)alkyl]-, —O—, —S—, —S(═O)— or —S(═O)₂—;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₅R₁₆—CR₁₇R₁₈—
wherein R₁₅, R₁₆, R₁₇ and R₁₈ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—,
and one or more therapeutically active co-agents selected from the group consisting of:
a) antidiabetic agents,
b) hypolipidemic agents,
c) anti-obesity agents,
d) anti-hypertensive agents, and
e) agonists of peroxisome proliferator-activator receptors.

56. A pharmaceutical compositions comprising:
i) a compound according to formula (III): 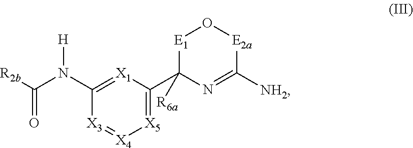
or a pharmaceutically acceptable salt thereof, wherein:
either
X₁ is CR₁ or N;
X₃ is CR₃ or N;
X₄ is CR₄ or N;
X₅ is CR₅ₐ or N;
wherein at least one of X₁, X₃, X₄ and X₅ is N and not more than 2 of X₁, X₃, X₄ and X₅ are N;
or
X₁ is CR₁ or N;
X₃ is CR₃, N or S;
X₄ is a bond;
X₅ is CR₅ₐ, N or S;
wherein at least one of X₁, X₃ and X₅ is N or S, not more than 2 of X₁, X₃ and X₅ are N and not more than 1 of X₃ and X₅ are S;
R₁ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R_{2b} is an aryl, heteroaryl or non-aromatic heterocyclyl group G₁, which group G₁ is optionally substituted by 1, 2, 3 or 4 substituents independently selected from the group, consisting of cyano, amino, amino-(C₁₋₈)alkyl, N—(C₁₋₄)alkyl-amino-(C₁₋₈)alkyl, N,N-di(C₁₋₄)alkyl-amino-(C₁₋₈)alkyl, aminocarbonyl, thiocarbamoyl, halogen, (C₁₋₈)alkyl, halogen-(C₁₋₈)alkyl, hydroxy, oxo, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₃₋₈)cycloalkyl-(C₁₋₈)alkoxy, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, (C₂₋₈)alkynyl, (C₂₋₈)alkenoxy, (C₂₋₈)alkynoxy and a (C₃₋₈)cycloalkyl, aryl, heteroaryl or non-aromatic heterocyclyl group G₂, which group G₂ is optionally substituted by 1, 2, 3, or 4 substituents independently selected from the group, consisting of cyano, aminocarbonyl, halogen, (C₁₋₈)alkyl, halogen-(C₁₋₈)alkyl, hydroxy, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl and (C₂₋₈)alkynyl;
R₃ is hydrogen, cyano, halogen, (C₁₋₈)alkyl, halogen-(C₁₋₈)alkyl, (C₁₋₈)alkoxy; halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₄ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
R₅ₐ is hydrogen, cyano, halogen, (C₁₋₈)alkyl, halogen-(C₁₋₈)alkyl, (C₁₋₈)alkoxy, halogen-(C₁₋₈)alkoxy, (C₁₋₈)alkylthio, halogen-(C₁₋₈)alkylthio, (C₁₋₈)alkoxy-(C₁₋₈)alkyl, (C₁₋₈)alkoxy-(C₁₋₈)alkoxy, (C₁₋₈)alkoxy-(C₁₋₈)alkylthio, (C₁₋₈)alkylthio-(C₁₋₈)alkyl, (C₁₋₈)alkylthio-(C₁₋₈)alkoxy, (C₁₋₈)alkylthio-(C₁₋₈)alkylthio, (C₂₋₈)alkenyl, or (C₂₋₈)alkynyl;
or
R₄ and R₅ₐ, taken together, are —C(H)═C(H)—C(H)═C(H)— or a (C₁₋₈)alkylene group, in which (C₁₋₈)alkylene group 1 or 2 —CH₂— ring members are optionally replaced with hetero ring members independently selected from the group, consisting of —N(H)—, —N[(C₁₋₈)alkyl]-, —O—, —S—, —S(═O)— or —S(═O)₂—;
R₆ₐ is hydrogen, (C₁₋₈)alkyl, halogen-(C₁₋₈)alkyl, hydroxy-(C₁₋₈)alkyl, (C₁₋₈)alkoxy-(C₁₋₈)alkyl, mercapto-(C₁₋₈)alkyl, (C₁₋₈)alkylthio-(C₁₋₈)alkyl, amino-(C₁₋₈)alkyl, N—(C₁₋₄)alkyl-amino-(C₁₋₈)alkyl, N,N-di(C₁₋₄)alkyl-amino-(C₁₋₈)alkyl, (C₂₋₈)alkenyl, or (C₂₋₈)alkynyl;
or
R₅ₐ and R₆ₐ, taken together, are a (C₁₋₄)alkylene group, in which (C₁₋₄)alkylene group 1 —CH₂— ring member is optionally replaced with a hetero ring member independently selected from the group, consisting of —N(H)—, —N[(C₁₋₄)alkyl]-, —O—, —S—, —S(═O)— or —S(═O)₂—;
E₁ is —C(R₇)(R₈)—, or —C(R₇)(R₈)—C(R₉)(R₁₀)—;
E₂ₐ is —C(R₁₁ₐ)(R₁₂ₐ)—, or —C(R₁₁ₐ)(R₁₂ₐ)—C(R₁₃)(R₁₄)—;
either
each of R₇ and R₈ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₇ and R₈, taken together, are oxo or —CH₂—CH₂—;
either
each of R₉ and R₁₀ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₉ and R₁₀, taken together, are oxo or —CH₂—CH₂—;
either
each of R₁₁ₐ and R₁₂ₐ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₁ₐ and R₁₂ₐ, taken together, are oxo or —CR₁₅R₁₆—CR₁₇R₁₈—
wherein R₁₅, R₁₆, R₁₇ and R₁₈ are independently selected from hydrogen and fluoro; and
either
each of R₁₃ and R₁₄ is independently selected from the group, consisting of hydrogen, cyano, halogen, (C₁₋₈)alkyl, halogen-(C₁₋₈)alkyl, (C₁₋₈)alkoxy-(C₁₋₈)alkyl and (C₁₋₈)alkylthio-(C₁₋₈)alkyl;
or
R₁₃ and R₁₄, taken together, are oxo or —CH₂—CH₂—, and
ii) at least one compound selected from
a) antidiabetic agents,
b) hypolipidemic agents,
c) anti-obesity agents,
d) anti-hypertensive agents,
e) agonists of peroxisome proliferator-activator receptors, and
iii) one or more pharmaceutically acceptable carriers.
